(19) United States
(12) Patent Application Publication
(10) Pub. No.: US 2026/0256789 A1
(43) Pub. Date: Sep. 3, 2026

(54) METHODS FOR TREATMENT OF CANCER

(71) Applicant: Frontier Medicines Corporation, South San Francisco, CA (US)

(72) Inventors: Snahel PATEL, Foster City, CA (US); Philip A. GERKEN, San Francisco, CA (US); Monika J. WILLIAMS, Woodside, CA (US)

(21) Appl. No.: 18/879,197

(22) PCT Filed: Jun. 29, 2023
(86) PCT No.: PCT/US2023/026589

§ 371 Date: Dec. 26, 2024

Related U.S. Application Data
(60) Provisional application No. 63/356,927, filed on Jun. 29, 2022.
(60) Provisional application No. 63/464,185, filed on May 4, 2023.
(60) Provisional application No. 63/465,510, filed on May 10, 2023.

(51) Int. Cl. A61K 31/519; C07D 487/04

(52) U.S. Cl. CPC A61K 31/519; C07D 487/04

(57) ABSTRACT

The present disclosure provides compounds and methods useful in the treatment and suppression of cancer, for example, useful for treating or suppressing cancers characterized by KRAS G12C. Also provided are pharmaceutical compositions containing such compounds and processes for preparing such compounds.

### CROSS-REFERENCE TO RELATED APPLICATIONS

This application claims the benefit of and priority to U.S. Provisional Patent Application No. 63/356,927, filed Jun. 29, 2022, U.S. Provisional Patent Application No. 63/464,185, filed May 4, 2023, and U.S. Provisional Patent Application No. 63/465,510, filed May 10, 2023, the entire disclosures of which are hereby incorporated by reference in their entirety for all purposes.

### FIELD OF THE DISCLOSURE

The present disclosure provides compounds useful in treating or suppressing cancer, and in particular, useful in treating or suppressing cancers characterized by the KRAS G12C mutant. Also provided are pharmaceutical formulations containing such compounds, processes for preparing such compounds, and methods of using such compounds in the treatment or suppression of cancers.

### BACKGROUND

KRAS is a molecular switch. Under normal physiological conditions, the protein is bound to guanosine diphosphate (GDP) in the “off state.” In response to signaling through receptor tyrosine kinases (RTKs) such as EGFR, the GDP is exchanged to guanosine triphosphate (GTP) in a process facilitated by guanine nucleotide exchange factors (GEFs) such as SOS. The GTP-bound form of KRAS is in the “on state,” and interacts with proteins such as RAF and PI3K to promote downstream signaling that leads to cell proliferation and survival. KRAS can slowly hydrolyze GTP back to GDP, thus returning to the off-state, in a process facilitated by GAPs (GTPase-activating Proteins).

KRAS mutations are found in approximately 30% of all human cancers, and are highly prevalent among three of the deadliest forms of cancer: pancreatic (95%), colorectal (45%), and lung (35%). Together, these cancers occur in more than 200,000 patients annually in the US alone. One particular mutation, a glycine to cysteine substitution at position 12 (G12C), occurs in more than 40,000 patients per year. The KRAS G12C mutation impairs hydrolysis of GTP to GDP, thus trapping KRAS in the on-state and promoting cancer cell proliferation.

The cysteine residue of G12C provides an opportunity to develop targeted covalent drugs for this mutant KRAS. Early clinical trial results for KRAS G12C inhibitors AMG 510 and MRTX849 have shown encouraging results for non-small cell lung cancer (NSCLC), but the data are less compelling for colorectal cancer (CRC). Moreover, even in cases where patients respond to initial treatment, there are signs that the response may be limited in duration and that resistance could arise rapidly.

Most inhibitors of KRAS mutants bind preferentially to the GDP-bound form of the protein. For example, Amgen KRAS inhibitor AMG 510 and Mirati KRAS inhibitor MRTX849 react with the GDP-bound form of KRAS G12C at least 1000-fold more rapidly than with the GTP-bound form of the protein. One form of resistance that has been observed is for cancer cells to increase signaling through RTKs, thus increasing the amount of GTP-bound KRAS, which is less affected by current inhibitors. Thus, creating a molecule that could bind to and inhibit both the GDP- and GTP-bound forms of KRAS could have substantial utility.

What is needed are compounds useful in the treatment of cancer, such as cancers characterized by KRAS G12C. What is further needed are compounds useful in the treatment of cancers characterized by KRAS G12C, wherein the compounds bind to and inhibit both the inactive GDP- and activated GTP-bound forms of KRAS. What is further needed are compounds useful in the treatment of cancers characterized by KRAS G12C, wherein the compound has improved inhibition of the GTP-bound form of KRAS G12C.

### SUMMARY

In a first aspect is a compound of Formula A, Formula B or Formula C:

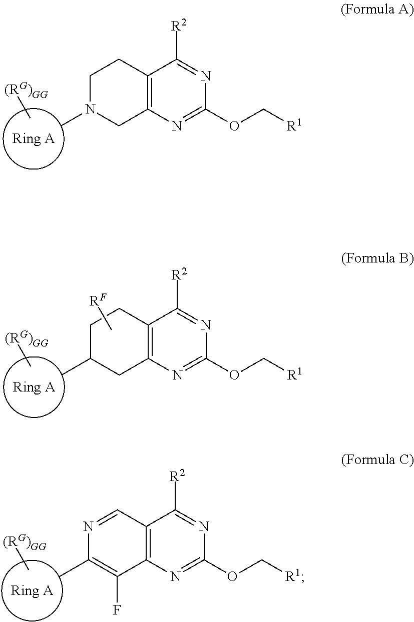

or a salt thereof; and/or an isotopologue thereof, wherein:
Ring A is a 6-membered aryl or a 5-10 membered heteroaryl;
R^{F} is selected from the group consisting of H, halo, C₁-C₄ alkyl, C₁-C₄ alkoxy, C₁-C₄ haloalkyl and C₁-C₄ haloalkoxy;
each R^{G} is independently selected from halo, —OH, —NH₂, C₁-C₄ alkyl, C₁-C₄ alkoxy, C₁-C₄ haloalkyl, C₁-C₄ haloalkoxy, C₃-C₆ cycloalkyl and C₂-C₃ alkynyl;
each GG is independently 0, 1, 2 or 3;
R¹ is a 4-8 membered saturated carbocyclic or heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy;
R² is selected from the group consisting of R^{2b}, R^{2c} and R^{2e};
R^{2b} is —NR¹⁰R¹¹;
R¹⁰ is selected from the group consisting of H, C₁-C₄ alkyl, C₁-C₄ alkoxy, C₁-C₄ haloalkyl, and C₁-C₄ haloalkoxy; and
R¹¹ is —(CH₂)_{w}—R¹³; or
R¹⁰ and R¹¹ together with the nitrogen to which they are attached form a 4-8 membered saturated heterocyclic group comprising a second nitrogen as the sole additional heteroatom within the ring atoms, wherein the second nitrogen of the 4-8 membered saturated heterocyclic group is substituted with cyano, and the 4-8 membered saturated heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
R¹³ is a 4-5 membered saturated heterocyclic group comprising one nitrogen as the sole heteroatom within the ring atoms, wherein the nitrogen is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo; or
R¹³ is a 6 membered saturated heterocyclic group comprising one or two nitrogens as the sole heteroatom(s) within the ring atoms, wherein one of the nitrogens is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom; or
R¹³ is a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom;
w is 0, 1, or 2;
R^{2c} is —NR¹⁵R¹⁶;
R¹⁵ is H, C₁-C₄ alkyl, C₁-C₄ alkoxy, C₁-C₄ haloalkyl, or C₁-C₄ haloalkoxy; and
R¹⁶ is —(CH₂)_{y}—R²¹;
R²¹ is selected from:
a 4-5 membered saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom; or
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom;
R¹⁸ is selected from the group consisting of hydrogen, —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)NR²²R²³, —(CH₂)_{z}—NR²²R²³, —(CH₂)ᵤ—R³⁴, —(C₁-C₂ alkyl)-(C₁-C₂ alkoxy), —S(O)₂—C₁-C₄ alkyl, —S(O)₂—C₁-C₄ haloalkyl, and R³⁵;
R¹⁹ is selected from the group consisting of hydrogen, C₁-C₄ alkyl, C₁-C₄ alkoxy, C₁-C₄ haloalkyl, and C₁-C₄ haloalkoxy;
R²⁰ is selected from the group consisting of hydrogen, C₁-C₄ alkyl, C₁-C₄ alkoxy, C₁-C₄ haloalkyl, and C₁-C₄ haloalkoxy;
R²² and R²³ are independently selected from the group consisting of hydrogen, C₁-C₄ alkyl, C₁-C₄ alkoxy, C₁-C₄ haloalkyl, and C₁-C₄ haloalkoxy;
R³⁴ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl;
R³⁵ is a 5-6 membered heteroaryl group optionally substituted with 0, 1 or 2 substituents independently selected from halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl;
y is 0, 1, or 2;
z is 1 or 2;
q is 0 or 1;
u is 0, 1 or 2;
R^{2e} is —NR²⁸R²⁹;
R²⁸ is H, C₁-C₄ alkyl, C₁-C₄ alkoxy, C₁-C₄ haloalkyl, or C₁-C₄ haloalkoxy; and
R²⁹ is —(CH₂)ₜ—R³⁰;
R³⁰ is selected from:
a 4-5 membered saturated heterocyclic group comprising one nitrogen as the sole heteroatom within the ring atoms, wherein the nitrogen ring atom of the heterocyclic group is substituted with —C(O)C≡CR³¹ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C≡CR³¹, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom; and
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C≡CR³¹, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom;
R³¹ is selected from the group consisting of —(CH₂)ᵥ—NR³²R³³ and —(CH₂)ₚ—R³⁶;
R³² and R³³ are independently selected from the group consisting of hydrogen, C₁-C₄ alkyl, C₁-C₄ alkoxy, C₁-C₄ haloalkyl, and C₁-C₄ haloalkoxy;
t is 0, 1, or 2;
v is 1 or 2;
p is 0, 1 or 2;
   R³⁶ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

In some embodiments, including any of the embodiments in the preceding paragraphs, the compound is selected from the group consisting of compounds of Table 1 and all salts and isotopologues thereof.

In another aspect provided is a pharmaceutical formulation comprising a compound as described herein, including but not limited to a compound described in the preceding paragraphs, and a pharmaceutically acceptable carrier, wherein when the compound is a salt, the salt is a pharmaceutically acceptable salt.

In another aspect provided is a method of treating or suppressing cancer comprising: administering a therapeutically effective amount of a compound as described herein, including but not limited to a compound described in the preceding paragraphs, or a pharmaceutical formulation, including but not limited to the pharmaceutical formulation described in the preceding paragraphs, to a subject in need thereof, wherein when the compound is a salt, the salt is a pharmaceutically acceptable salt. In some embodiments, the cancer is selected from the group consisting of: lung, colorectal, pancreatic, bile duct, thyroid, gall bladder, uterine, mesothelioma, cervical, and bladder cancers. In some embodiments, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the method is for treating the cancer. In some embodiments, including any of the foregoing embodiments, the method is for suppressing the cancer. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, the method further comprises administering to the subject a therapeutically effective amount of an additional chemotherapeutic agent.

In another aspect provided is the use of a compound as described herein, including but not limited to any of the foregoing embodiments, as a medicament. In another aspect is the use of a compound as described herein, including but not limited to any of the foregoing embodiments, for treating or suppressing cancer. In another aspect is the use of a compound as described herein, including but not limited to any of the foregoing embodiments, in the manufacture of a medicament for use in treating or suppressing cancer. In some embodiments, including any of the foregoing embodiments, the use is for treating the cancer. In some embodiments, including any of the foregoing embodiments, the use is for suppressing the cancer.

In another aspect provided is a compound as described herein, including but not limited to any of the foregoing embodiments for use in the manufacturing of a medicament for treating or suppressing cancer. In another aspect is a compound as described herein, including but not limited to any of the foregoing embodiments, for use in treating or suppressing cancer. In another aspect is the compound as described herein, including but not limited to any of the foregoing embodiments, for use in the manufacture of a medicament for treating or suppressing cancer. In some embodiments, including any of the foregoing embodiments, the use is for treating the cancer. In some embodiments, including any of the foregoing embodiments, the use is for suppressing the cancer.

It is to be understood that the description of compounds, compositions, formulations, and methods of treatment described herein include “comprising”, “consisting of”, and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

Additional embodiments, features, and advantages of the present disclosure will be apparent from the following detailed description and through practice of the present disclosure.

### DETAILED DESCRIPTION

Provided herein are compounds useful in treating cancer, and methods of using such compounds for treating cancer. In some embodiments, the compounds are useful in treating cancers characterized by KRAS G12C. In some embodiments, the compounds advantageously inhibit both the inactive GDP- and activated GTP-bound forms of KRAS G12C. In some embodiments, the compounds advantageously have improved inhibition of the GTP-bound form of KRAS G12C.

The abbreviations used herein have their conventional meaning within the chemical and biological arts, unless otherwise specified.

It is to be understood that descriptions of compound structures, including possible substitutions, are limited to those which are chemically possible.

Unless otherwise indicated, the absolute stereochemistry of all chiral atoms is as depicted. Compounds with an (or) designation in the first column of Table 1 are single enantiomers wherein the absolute stereochemistry was arbitrarily assigned (e.g., based on chiral SFC elution as described in the Examples section) and unless otherwise specified, the relative stereochemistry is as shown. Compounds with an (and) designation in the first column of Table 1 are mixtures of enantiomers wherein the relative stereochemistry is as shown. Compounds that have a stereogenic center where the configuration is not indicated in the structure as depicted and that have no designation in the first column of Table 1 are mixtures of enantiomers at that center. Compounds that have a stereogenic center where the configuration is indicated by wedges or hashes in the structure, and that have no designation in the first column of Table 1 or that are marked with (abs) are single enantiomers wherein the absolute stereochemistry is as indicated. For example, compound 1 is a pure enantiomer with the stereochemistry as indicated.

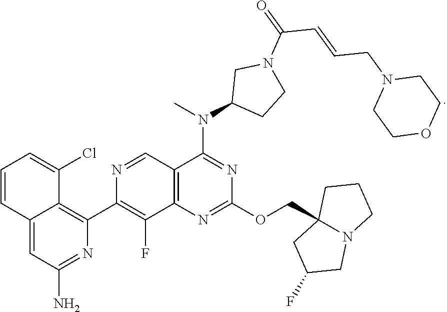 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/CN2CCOCC2)C1]

In some instances, the first column of Table 1 contains different indicators selected from (abs) (or) and (and) to refer to different stereocenters or pairs of stereocenters of the molecule.

For example, Compound 9 includes a notation of “(abs) pyrrolidine, (or) both cyclohexenyl stereocenters” in column 1 of Table 1.

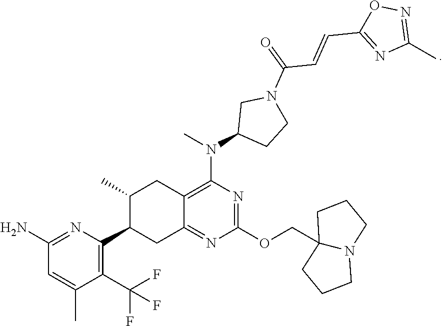 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1]

The compound is a single enantiomer wherein the stereochemistry at the pyrrolidine group is (S) as shown, because the pyrrolidine group was prepared from an enantiopure starting material, and the stereochemistry at the fused cyclohexenyl is either (R,R) or (S,S), but not a mixture of the two, and not a mixture with (R,S) or (S,R); the stereochemistry was arbitrarily assigned. Stereochemistry is often arbitrarily assigned when mixtures of enantiomers or diastereomers are separated into the corresponding single enantiomers or diastereomers by chromatography.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” and “an,” as used in herein mean one or more, unless context clearly dictates otherwise.

The terms “subject,” “individual,” and “patient” mean an individual organism, preferably a vertebrate, more preferably a mammal, most preferably a human. Examples of patients include humans, livestock such as cows, goats, sheep, pigs, and rabbits, and companion animals such as dogs, cats, and horses. In some embodiments, the subject has been identified or diagnosed as having a cancer or tumor having a KRAS G12C mutation (e.g., as determined using a regulatory agency-approved, e.g., FDA-approved, assay or kit).

“Treating” a disorder with the compounds and methods discussed herein is defined as administering one or more of the compounds discussed herein, with or without additional therapeutic agents, in order to reduce or eliminate either the disorder or one or more symptoms of the disorder, or to retard the progression of the disorder or of one or more symptoms of the disorder, or to reduce the severity of the disorder or of one or more symptoms of the disorder.

“Suppression” of a disorder with the compounds and methods discussed herein is defined as administering one or more of the compounds discussed herein, with or without additional therapeutic agents, in order to suppress the clinical manifestation of the disorder, or to suppress the manifestation of adverse symptoms of the disorder. The distinction between treatment and suppression is that treatment occurs after adverse symptoms of the disorder are manifest in a subject, while suppression occurs before adverse symptoms of the disorder are manifest in a subject. Suppression may be partial, substantially total, or total. In some embodiments, genetic screening can be used to identify patients at risk of the disorder. The compounds and methods disclosed herein can then be administered to asymptomatic patients at risk of developing the clinical symptoms of the disorder, in order to suppress the appearance of any adverse symptoms.

“Therapeutic use” of the compounds discussed herein is defined as using one or more of the compounds discussed herein to treat or suppress a disorder, as defined herein. A “therapeutically effective amount” of a compound is an amount of the compound, which, when administered to a subject, is sufficient to reduce or eliminate either the disorder or one or more symptoms of the disorder, or to retard the progression of the disorder or of one or more symptoms of the disorder, or to reduce the severity of the disorder or of one or more symptoms of the disorder, or to suppress the clinical manifestation of a disorder, or to suppress the manifestation of adverse symptoms of a disorder. A therapeutically effective amount can be given in one or more administrations.

A “KRAS G12C mediated cancer” is used interchangeably herein with a “cancer characterized by KRAS G12C”, and indicates that the cancer comprises cells which contain the KRAS G12C mutant.

While the compounds described herein can occur and can be used as the neutral (non-salt) compound, the description is intended to embrace all salts of the compounds described herein, as well as methods of using such salts of the compounds. In some embodiments, the salts of the compounds comprise pharmaceutically acceptable salts.

A “pharmaceutically acceptable salt” of a compound means a salt that is pharmaceutically acceptable to humans and/or animals, and which, upon administration, retains at least some of the desired pharmacological activity of the parent compound. Such salts include: (a) acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (b) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Additional information on suitable pharmaceutically acceptable salts can be found in Remington's Pharmaceutical Sciences, 17th ed., Mack Publishing Company, Easton, PA, 1985, which is incorporated herein by reference in its entirety.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

“Isotopologue” refers herein to a compound which differs in its isotopic composition from its “natural” isotopic composition. “Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition. The description of compounds herein also includes all isotopologues, in some embodiments, partially deuterated or perdeuterated analogs, of all compounds herein. “Isotopically enriched” may also refer to a compound containing at least one atom having an isotopic composition other than the natural isotopic composition of that atom. “Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Alkyl” means a linear, branched, cyclic, or a combination thereof, saturated monovalent hydrocarbon radical having the defined number of carbons. For example, C₁-C₄ alkyl includes e.g., methyl, ethyl, propyl, 2-propyl, butyl, cyclopropyl, cyclobutyl, and the like.

“Alkylene” means a linear, branched, cyclic, or a combination thereof, saturated divalent hydrocarbon radical having the defined number of carbons. For example, C₁-C₄ alkylene includes e.g., methylene, ethylene, propylene, 1-methylpropylene, 2-methylpropylene, butylene, and the like. “C₀ alkylene” means a bond. For example, C₀-C₂ alkylene includes a bond, methylene, ethylene, and the like.

“Alkynyl” means a linear or branched monovalent hydrocarbon radical having the defined number of carbons and at least one carbon-carbon triple bond. For example, C₂-C₄ alkyne includes e.g., ethynyl, propynyl, 2-propynyl, butynyl, and the like.

“Alkoxy” means an —OR^{o} radical where R^{o} is alkyl as defined above, or a R^{o′}OR^{o″} radical where R^{o′} is an alkylene and and R^{o″} is an alkyl group as defined above where the defined number of alkyl carbons in the alkoxy group are equal to the total number of carbons in R^{o′} and R^{o″}. For example, C₁-C₄ alkoxy indicates e.g., methoxy, ethoxy, propoxy, 2-propoxy, n-, iso-, tert-butoxy, cyclopropoxy, methoxymethyl, ethoxymethyl, propoxymethyl, isopropoxymethyl, and the like. In some embodiments, alkoxy is a —OR^{o} radical. In some embodiments, alkoxy is a —R^{o′}OR^{o″} radical. In some embodiments, when a nitrogen is substituted with an alkoxy group, the alkoxy group is not linked to the nitrogen via the oxygen or a carbon that is immediately adjacent to the oxygen in the alkoxy group. For example, the alkoxy-substituted nitrogen is not N—OR^{o} or N—CH₂—O—R^{o″}.

“Alkoxyalkoxy” means an —OR^{r} radical where R is alkoxy as defined above, provided that the attachment point of R is not an oxygen atom, or a —R^{r′}OR^{r″} radical where R^{r} is an alkylene and R^{r″} is an alkoxy group as defined above, provided that the attachment point of R^{r″} is not an oxygen atom, where the defined number of alkyl carbons in the alkoxyalkoxy group are equal to the total number of carbons in R^{r′} and R^{r″}. For example, C₁-C₆ alkoxyalkoxy indicates e.g., —OCH₂OCH₃, —OCH₂CH₂OCH₃, —OCH₂CH₂OCH₃, —CH₂OCH₂OCH₃, —CH₂OCH₂CH₂OCH₃, —CH₂OCH₂CH₂OCH₂CH₃, —CH₂CH₂OCH₂CH₂OCH₂CH₃ and the like. In some embodiments, alkoxyalkoxy is a —OR^{r} radical. In some embodiments, alkoxyalkoxy is a —R^{r′}OR^{r″} radical. In some embodiments, when a nitrogen is substituted with an alkoxyalkoxy group, the alkoxyalkoxy group is not linked to the nitrogen via the oxygen or a carbon that is immediately adjacent to the oxygen in the alkoxyalkoxy group. For example, the alkoxyalkoxy-substituted nitrogen is not N—OR^{r} or N—CH₂—O—R^{r″}.

“Aminoalkyl” means an —NHRⁿ radical where Rⁿ is alkyl as defined above, or a —NRⁿR^{n′} radical where Rⁿ and R^{n′} are alkyl groups as defined above, or an —R^{n″}NH₂ radical where R^{n″} is an alkylene group as defined above, or an —R^{n″}NHRⁿ radical where R^{n″} is an alkylene group as defined above and R¹¹ is an alkyl group as defined above, or a —R^{n″}NRⁿR^{n′} radical where R^{n″} is an alkylene group as defined above and Rⁿ and R^{n′} are alkyl groups as defined above, where the defined number of alkyl carbons in the aminoalkyl group is equal to the total number of carbons in Rⁿ, R^{n′} and R^{n″} as applicable. For example, C₁-C₆ aminoalkyl indicates e.g., —NHCH₃, —NHCH₂CH₃, —NHCH₂(CH₃)₂, —N(CH₃)₂, —N(CH₃)CH₂CH₃, —N(CH₂CH₃)₂, —CH₂NH₂, —CH₂CH₂NH₂, —CH₂NHCH₃, —CH₂N(CH₃)₂, —CH₂CH₂NHCH₃, —CH₂CH₂N(CH₃)₂ and the like. In some embodiments, aminoalkyl is an —NHRⁿ radical. In some embodiments, aminoalkyl is an —NRⁿR^{n′} radical. In some embodiments, an aminoalkyl is an —R^{n″}NH₂ radical. In some embodiments, aminoalkyl is a —R^{n″}NHRⁿ radical. In some embodiments, aminoalkyl is a —R″NRⁿR^{n′} radical. In some embodiments, when an oxygen is substituted with an aminoalkyl group, the aminoalkyl group is not linked to the oxygen via the nitrogen or a carbon that is immediately adjacent to the nitrogen in the aminoalkyl group. For example, the aminoalkyl-substituted oxygen is not O—NRⁿ or O—CH₂—NHRⁿ. “Aryl” refers to a radical of a monocyclic or polycyclic (e.g., bicyclic or tricyclic) aromatic ring system having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). In some embodiments, “aryl” also includes ring systems wherein the aryl ring, as defined above, is fused with one or more carbocyclyl or heterocyclyl groups wherein the radical or point of attachment is on the aryl ring, and in such instances, the number of ring carbon atoms continue to designate the number of ring carbon atoms in the aryl ring system. Exemplary aryl groups include phenyl and naphthyl, wherein the attachment point can be on any carbon atom. Exemplary aryl groups also include indenyl, tetrahydronaphthyl, indolinyl, benzodihydrofuranyl, tetrahydroquinolinyl, tetrahydroisoquinolinyl and the like, wherein the attachment point is on the phenyl group. In some embodiments, “aryl” excludes ring systems wherein the aryl ring, as defined above, is fused with one or more carbocyclyl or heterocyclyl groups.

“Cycloalkyl” means a monocyclic saturated monovalent hydrocarbon radical having the defined number of carbon atoms. For example, C₃-C₆ cycloalkyl includes cyclopropyl, cyclobutyl, cyclopentyl, and cyclohexyl.

“Cyanoalkyl” means an alkyl radical as defined above, which is substituted with a cyano group (—CN). A cyanoalkyl can also be referred to as an alkylnitrile.

“Halo” means fluoro, chloro, bromo, or iodo. In some embodiments, halo is fluoro or chloro.

“Haloalkyl” means an alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means an —OR^{a} radical where R^{a} is haloalkyl as defined above, or a —R^{b}OR^{c} radical where R and R are alkyl or haloalkyl groups as defined above where the defined number of alkyl carbons in the haloalkoxy group are equal to the total number of carbons in R^{b} and R. Halo atom(s) may be present in R, or R, or both, provided that at least one of R^{b} and R comprises a halo atom. For example, C₁-C₄ haloalkoxy indicates e.g., —OCF₃, —OCHF₂, —CH₂OCF₃, —CH₂CH(F)CH₂OCH₃, —CH₂CH(F)CH₂OCHF₂, and the like. In some embodiments, haloalkoxy is a —OR^{a} radical. In some embodiments, haloalkoxy is a —R^{b}OR^{c} radical. When all of the halo atom(s) in the haloalkoxy group are fluoro, it can be referred to in this Application as fluoroalkoxy. In some embodiments, when a nitrogen is substituted with a haloalkoxy group, the haloalkoxy group is not linked to the nitrogen via the oxygen or a carbon that is immediately adjacent to the oxygen in the haloalkoxy group. For example, the haloalkoxy-substituted nitrogen is not N—OR^{a} or N—C(H)ₙ(X)ₘ—O—R^{c}(wherein X is a halogen and n and m are integers, provided that n+m=2).

“Hydroxyalkyl” means an alkyl radical as defined above, which is substituted with one or more hydroxyl (—OH) groups, e.g., one to three hydroxyl groups, e.g., —CH₂OH, —CH₂CH₂OH, —C(OH)(CH₃)₂, —CH(OH)CH₃ and the like.

A “heterocyclic group” or “heterocycle”, unless otherwise specified, means a saturated or partially unsaturated cyclic group comprising 3-12 ring atoms, in which 1-4 ring atoms are heteroatoms independently selected from the group consisting of nitrogen, oxygen, and sulfur, the remaining rings being C. The sulfur group may be present either as —S— or as —S(O)₂—. Unless otherwise specified, the heterocyclic group includes single as well as multiple ring systems including fused, bridged, and spiro ring systems. “Heterocyclic group” or “heterocycle” includes ring systems wherein the heterocyclic group, as defined above, is fused with one or more carbocyclic groups wherein the point of attachment is either on the carbocycle or heterocycle ring, and in such instances, the number of ring members continue to designate the number of ring members in the heterocyclyl ring system. In some embodiments, “heterocyclic group” or “heterocycle” also includes ring systems wherein the heterocyclic group, as defined above, is fused with one or more aryl or heteroaryl groups, wherein the point of attachment is on the heterocyclyl ring, and in such instances, the number of ring members continue to designate the number of ring members in the heterocyclyl ring system. In some embodiments, “heterocyclic group” or “heterocycle” excludes ring systems wherein the heterocyclic group, as defined above, is fused with one or more carbocyclic, aryl or heteroaryl groups. In some embodiments, the heterocyclic group is a single ring. In some embodiments, the heterocyclic group comprises two fused rings. In some embodiments, the heterocyclic group comprises two spiro rings. In some embodiments, the heterocyclic group comprises a bridged ring system.

A “carbocyclic group” or “carbocycle”, unless otherwise specified, means a saturated or partially unsaturated cyclic group comprising 3-12 ring atoms, in which the ring atoms are C. Unless otherwise specified, the carbocyclic group includes single as well as multiple ring systems including fused, bridged, and spiro ring systems. In some embodiments, the carbocyclic group is a single ring. In some embodiments, the carbocyclic group comprises two fused rings. In some embodiments, the carbocyclic group comprises two spiro rings. In some embodiments, the carbocyclic group comprises a bridged ring system.

“Heteroaryl” means a monovalent monocyclic or bicyclic aromatic radical of 5 to 10 ring atoms, unless otherwise stated, where one or more (in some embodiments, one, two, or three) ring atoms are heteroatom(s) independently selected from N, O, or S, the remaining ring atoms being carbon. “Heteroaryl” includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) can have the point of attachment on either ring, i.e., either the ring bearing a heteroatom (e.g., 2-indolyl) or the ring that does not contain a heteroatom (e.g., 5-indolyl). In some embodiments, “heteroaryl” includes ring systems wherein the heteroaryl ring, as defined above, is fused with one or more carbocyclyl or heterocyclyl groups wherein the point of attachment is on the heteroaryl ring. In such instances, unless otherwise specified, the number of ring members continue to designate the number of ring members in the heteroaryl ring system. In some embodiments, “heteroaryl” excludes ring systems wherein the heteroaryl ring is fused with a carbocyclyl or heterocyclyl group. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

A “spiro” cycloalkyl group indicates that the cycloalkyl group is linked to the remaining portion of the compound through a spiro linkage. A “spiro” cycloalkyl substituent has two attachments that connect to the same carbon of the moiety that is substituted, forming a spiro connection. For example, a cyclohexyl group that is substituted with a “spiro C₃-C₄ cycloalkyl” group indicates:

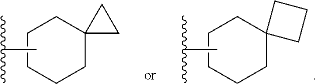

“In need of treatment” as used herein means the patient is being treated by a physician or other caregiver after diagnoses of the disease, or a determination that the patient is at risk for developing the disease. In some embodiments, the patient has been diagnosed as having a KRAS G12C mediated cancer. In some embodiments, the patient has been determined to be at risk of developing a KRAS G12C mediated cancer.

“Administration” “administer” and the like, as they apply to, for example, a patient, cell, tissue, organ, or biological fluid, refer to contact of, for example, a compound of Formula (A), Formula (B) or Formula (C), or a pharmaceutically acceptable salt and/or isotopologue thereof, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Optional” or “optionally” means that the subsequently described event or circumstance may but need not occur, and that the description includes instances where the event or circumstance occurs and instances in which it does not.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule or a tablet having a fixed ratio of active ingredients or in multiple, separate capsules or tablets for each active ingredient. In addition, such administration also encompasses use of each type of therapeutic agent in a sequential manner. In either case, the treatment regimen will provide beneficial effects of the drug combination in treating the conditions or disorders described herein.

### Methods for Treatment of Cancer

The compounds of Formula (A), Formula (B) and Formula (C), and pharmaceutically acceptable salts and/or isotopologues thereof, including embodiments thereof disclosed herein, are useful for the treatment of cancer, which include but are not limited to, various types of cancer including e.g. lung, colorectal, pancreatic, bile duct, thyroid, gall bladder, uterine, mesothelioma, cervical, and bladder cancers. More particularly, cancers that may be treated by the compounds of Formula (A), Formula (B) and Formula (C), and pharmaceutically acceptable salts and/or isotopologues thereof, including embodiments thereof disclosed herein, include, but are not limited to cancers such as glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, including any of the foregoing embodiments, the subject has been determined to be at risk of developing a KRAS G12C mediated cancer.

In an aspect, provided is a compound of Formula (A), Formula (B) or Formula (C) as described in any of the embodiments described herein or a pharmaceutical formulation as described in any of the embodiments described herein for use as a medicament.

In an aspect, provided is a compound of Formula (A), Formula (B) or Formula (C) as described in any of the embodiments described herein or a pharmaceutical formulation as described in any of the embodiments described herein for use in treating or suppressing cancer. In an embodiment, when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical formulation is configured for administration with a therapeutically effective amount of an additional chemotherapeutic agent. In an embodiment, the compound or pharmaceutical formulation is configured for administration in a therapeutically effective amount.

In an aspect, provided is a compound of Formula (A), Formula (B) or Formula (C) as described in any of the embodiments described herein or a pharmaceutical formulation as described in any of the embodiments described herein for use in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical formulation is configured for administration with a therapeutically effective amount of an additional chemotherapeutic agent. In an embodiment, the medicament comprises a therapeutically effective amount of the compound or pharmaceutical formulation.

In an aspect, provided is a use of a compound of Formula (A), Formula (B) or Formula (C) as described in any of the embodiments described herein or a pharmaceutical formulation as described in any of the embodiments described herein in the manufacturing of a medicament for treating or suppressing cancer, wherein when the compound is a salt, the salt is a pharmaceutically acceptable salt. In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical formulation is configured for administration with a therapeutically effective amount of an additional chemotherapeutic agent. In an embodiment, the medicament comprises a therapeutically effective amount of the compound or pharmaceutical formulation.

In an aspect, provided is a use of a compound of Formula (A), Formula (B) or Formula (C) as described in any of the embodiments described herein or a pharmaceutical formulation as described in any of the embodiments described herein for treating or suppressing cancer, wherein when the compound is a salt, the salt is a pharmaceutically acceptable salt.

In an embodiment, the cancer is selected from the group consisting of: lung, colorectal, pancreatic, bile duct, thyroid, gall bladder, uterine, mesothelioma, cervical, and bladder cancers. In an embodiment, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In an embodiment, the cancer is a KRAS G12C mediated cancer. In an embodiment, the subject has been diagnosed as having a KRAS G12C mediated cancer. In an embodiment, the compound or pharmaceutical formulation is configured for administration with a therapeutically effective amount of an additional chemotherapeutic agent. In an embodiment, use involves a therapeutically effective amount of the compound or composition.

In some embodiments, including any of the foregoing embodiments, the subject and/or the cancer is resistant or refractory to treatment with certain KRAS inhibitors (e.g., G12C KRAS inhibitors).

The compounds of Formula (A), Formula (B) or Formula (C), and pharmaceutically acceptable salts and/or isotopologues thereof, including embodiments thereof disclosed herein, may be used for methods for inhibiting KRAS G12C in a cell, by contacting the cell in which inhibition of KRAS G12C activity is desired with an amount of the compound effective to inhibit KRAS G12C activity. Inhibition may be partial or total. In some embodiments, the contacting is in vitro. In some embodiments, the contacting is in vivo.

### Testing

The compounds of Formula (A), Formula (B) or Formula (C), and pharmaceutically acceptable salts and/or isotopologues thereof, including embodiments thereof disclosed herein, may be tested by, for example, methods described in the Examples below, or by known and generally accepted cell and/or animal models.

The ability of compounds of Formula (A), Formula (B) and Formula (C), and pharmaceutically acceptable salts and/or isotopologues thereof, to inhibit activity of the GTP-bound form of KRAS G12C can be tested using methods such as the in vitro assay described in Example 179 below. Example 179 describes determining, for various compounds, the half-maximal inhibition (IC₅₀) of KRAS G12C loaded with GTP analogue GMPPNP from binding to cRaf, as the Ras-binding domain (RBD). Example 180 describes determining, for various compounds, the half-maximal inhibition (IC₅₀) of KRAS G12C loaded with GTP analogue GMPPNP from binding to PI3Kα, as the Ras-binding domain (RBD). Example 181 describes testing compounds for the ability to inhibit cell viability in MCF10A G12C/A59G mutant, which abrogates GTPase activity, thus preventing hydrolysis of GTP to GDP.

### Pharmaceutical Compositions

The terms pharmaceutical composition and pharmaceutical formulation are used interchangeably throughout.

In general, the compounds of Formula (A), Formula (B) and Formula (C), and pharmaceutically acceptable salts and/or isotopologues thereof, of this disclosure (also may be referred to herein as “compounds” or “compounds of this disclosure”) will be administered in a therapeutically effective amount by any of the accepted modes of administration for agents that serve similar utilities. Therapeutically effective amounts of compounds of this disclosure may range from about 0.01 to about 500 mg per kg patient body weight per day, which can be administered in single or multiple doses. In some embodiments, a suitable dosage level may be from about 0.1 to about 250 mg/kg per day; or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of a compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the disclosure may be administered topically, that is by non-systemic administration. This includes the application of the compounds externally to the epidermis or the buccal cavity and the instillation of such compounds into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.0010% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.10% to 10% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

### Combinations and Combination Therapies

The compounds of this disclosure may be used in combination with one or more other drugs in the treatment of diseases or conditions for which compounds of this disclosure or the other drugs may have utility. Such other drug(s) may be administered contemporaneously or sequentially with a compound of the present disclosure. When a compound of this disclosure is used contemporaneously with one or more other drugs, a pharmaceutical composition in unit dosage form containing such other drugs and the compound of the present disclosure is contemplated. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, a therapeutically effective dose of each will be used.

Where the subject in need is suffering from or at risk of suffering from cancer, the subject can be treated with a compound of this disclosure in any combination with one or more other anti-cancer agents.

In some embodiments, the compounds of the present disclosure are used in combination with a CDK 4/6 inhibitor. Examples of CDK 4/6 inhibitors suitable for the provided compositions and methods include, but are not limited to, abemaciclib (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine); palbociclib (6-acetyl-8-cyclopentyl-5-methyl-2-((5-(piperazin-1-yl)pyridin-2-yl)amino)-pyrido[2,3-d]pyrimidin-7(8H)-one) and ribociclib (7-cyclopentyl-N,N-dimethyl-2-((5-(piperazin-1-yl)pyridin-2-yl)amino)-7H-pyrrolo[2,3-d]pyrimidine-6-carboxamide) whereas the CDK 4/6 inhibitor trilaciclib (2′-((5-(piperazin-1-yl)pyridin-2-yl)amino)-7′,8′-dihydro-6′H-spiro-[cyclohexane-1,9′-pyrazino[1′,2′:1,5]pyrrolo[2,3-d]pyrimidin]-6′-one) is in late stage clinical trials Another CDK 4/6 inhibitor useful in the methods herein is the CDK 2/4/6 inhibitor PF-06873600 (pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In another embodiment the compounds of the present disclosure are used in combination with Raf family kinase inhibitors. Examples of Raf family kinase inhibitors suitable for the provided compositions and methods include, but are not limited to, encorafenib (LGX818): methyl (S)-(1-((4-(3-(5-chloro-2-fluoro-3-(methylsulfonamido)phenyl)-1-isopropyl-1H-pyrazol-4-yl)pyrimidin-2-yl)anno)propan-2-yl)carbamate; PLX-8394: N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide; Raf-709: N-(2-methyl-5′-morpholino-6′-((tetrahydro-2H-pyran-4-yl)oxy)-[3,3′-bipyridin]-5-yl)-3-(trifluoromethyl)benzamide. LXH254: N-(3-(2-(2-hydroxyethoxy)-6-morpholinopyridin-4-yl)-4-methylphenyl)-2-(trifluoromethyl)isonicotinamide; Sorafenib: 4-(4-(3-(4-chloro-3-(trifluoromethyl)phenyll)ureido)phenoxy)-N-methylpicolinamide; L Y 3009120: 1-(3,3-dimethylbutyl)-3-(2-fluoro-4-methyl-5-(7-methyl-2-(methylamino)pyrido-[2,3-d]pyrimidin-6-yl)phenyl)urea; Lifirafenib (BGB-283); 5-(((1R,1aS,6bS)-1-(6-(trichloro-methyl)-1H-benzo[d]imidazol-2-yl)-1a,6b-dihydro-1H-cyclopropa[b]benzofuran-5-yl)methyl)-3,4-dihydro-1,8-naphthyridin-2(1H)-one; Tak-632: N-(7-cyano-6-(4-fluoro-3-(2-(3-(trifluoromethyl)-phenyl)acetamido)phenoxy)benzo[d]thiazol-2-yl)cyclopropanecarboxamide; CEP-32496: 143-((6,7-dimethoxyquinazolin-4-yl)oxy)phenyl)-3-(5-(1,1,1-trifluoro-2-methylpropan-2-yl)isoxazol-3-yl)urea; CCT196969: 1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea; and R05126766: N-[3-fluoro-4-[[4-methyl-2-oxo-7-(2-pyrimidinyloxy)-2H-1-benzopyran-3-yl]methyl]-2-pyridinyl]-N′-methylsulfamide.

In another embodiment the compounds of the present disclosure are used in combination with Src family kinases. Examples of Src family kinase inhibitors suitable for the provided compositions and methods include, but are not limited to, Dasatinib (N-(2-chloro-6-methylphenyl)-2-((6-(4-(2-hydroxyethyl)piperazin-1-yl)-2-methylpyrimidin-4-yl)amino)thiazole-5-carboxamide); Ponatinib (3-(imidazo[1,2-b]pyridazin-3-ylethynyl)-4-methyl-N-(4-((4-methylpiperazin-1-yl)methyl)-3-(trifluoromethyl)phenyl)benzamide); Vandetanib (N-(4-bromo-2-fluorophenyl)-6-methoxy-7-((1-methylpiperidin-4-yl)methoxy)quinazolin-4-amine); Bosutinib (4-((2,4-dichloro-5-methoxyphenyl)amino)-6-methoxy-7-(3-(4-methylpiperazin-1-yl)-propoxy)quinoline-3-carbonitrile); Saracatinib (N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine); KX2-391 (N-benzyl-2-(5-(4-(2-morpholinoethoxy)phenyl)pyridin-2-yl)acetamide); SU6656 ((Z)—N,N-dimethyl-2-oxo-3-((4,5,6,7-tetrahydro-1H-indol-2-yl)methylene)indoline-5-sulfonamide); PP1 (1-(tert-butyl)-3-(p-tolyl)-1H-pyrazolo[3,4-d]pyrimidin-4-amine); WH-4-023 (2,6-dimethylphenyl (2,4-dimethoxyphenyl)(2-((4-(4-methylpiperazin-1-yl)phenyl)amino)pyrimidin-4-yl)carbamate) and KX-01 (N-benzyl-2-(5-(4-(2-morpholinoethoxy)phenyl)pyridin-2-yl)acetamide). In one embodiment, the Src inhibitor is Dasatinib. In one embodiment, the Src inhibitor is Saracatinib. In one embodiment, the Src inhibitor is Ponatinib. In one embodiment, the Src inhibitor is Vandetanib. In one embodiment, the Src inhibitor is KX-01.

In another embodiment the compounds of the present disclosure are used in combination with a SHP-2 inhibitor which include, but are not limited to SHP-099 (6-(4-amino-4-methylpiperidin-1-yl)-3-(2,3-dichlorophenyl)pyrazine-2-amine dihydrochloride), RMC-4550 (3(3S,4S)-(4-amino-3-methyl-2-oxa-8-azaspiro[4.5]decan-8-yl)-6-(2,3-dichlorophenyl)pyrazin-2-yl)methanol), RMC-4360 (Revolution Medicine), TN0155 (Novartis), BBP-398 (BridgeBio), and ERAS-601 (Erasca).

In another embodiment the compounds of the present disclosure are used in combination with an mTOR inhibitor. Examples of mTOR inhibitors suitable for the provided compositions and methods include, but are not limited to. Everolimus, Rapamycin. Zotarolimus (ABT-578), ridaforolimus (Deforolimus; MK-8669), Sapanisertib (INK128; 5-(4-amino-1-isopropyl-1H-pyrazolo[3,4-d]pyrimidin-3-yl)benzo[d]oxazol-2-amine), Torin-1; 1-(4-(4-propionylpiperazin-1-yl)-3-(trifluoromethyl)cyclohexyl)-9-(quinolin-3-yl)benzo[h][1,6]naphthyridin-2(1H)-one, dactolisib (BEZ235); 2-methyl-2-(4-(3-methyl-2-oxo-8-(quinolin-3-yl)-2,3-dihydro-1H-imidazo[4,5-c]quinolin-1-yl)phenyl)propanenitrile, buparlisib (5-(2,6-dimorpholin-4-ylpyrimidin-4-yl)-4-(trifluormethyl)pyridin-2-amine); GDC-0941 (pictilisib); 4-[2-(1H-indazol-4-yl)-6-[(4-methylsulfonylpiperazin-1-yl)methyl]thieno[3,2-d]pyrimidin-4-yl]morpholine); GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea); VS-5584 (SB2343) (5-(8-methyl-2-morpholin-4-yl-9-propan-2-ylpurin-6-yl)pyrimidin-2-amine) and vistusertib (AZD-2014; 3-(2,4-bis((S)-3-methylmorpholino)pyrido-[2,3-d]pyrimidin-7-yl)-N-methylbenzamide).

In another embodiment the compounds of the present disclosure are used in combination with a pan ErbB family inhibitor. In one embodiment the KRAS and pan ErbB family inhibitors are the only active agents m the provided compositions and methods. In one embodiment, the pan ErbB family inhibitor is an irreversible inhibitor. Examples of irreversible pan ErbB family inhibitors suitable for the provided compositions and methods include, but are not limited to, Afatinib; Dacomitinib; Canertinib; Poziotinib, AV 412 (N-4-([3-(chloro-4-fluorophenyl)amino]-7-[3-methyl-3-(4-methyl-1-piperazin-1-butyn-1-yl]-6-quinazolinyl]-2-prepenamide); PF 6274484 N-4-([3-(chloro-4-fluorophenyl)amino]-7-methoxy-6-quinazolinyl]-2-propenamide) and HKI 357 N-(2(E)-N-[[4-[[3-chloro-4-[(fluorophenyl)methoxy]phenyl]amino]-3-cyano-7-ethoxy-6-quinolinyl]-4-(dimethylamino)-2-butenamide). In another embodiment, the pan ErbB family inhibitor is a reversible inhibitor. Examples of reversible pan ErbB family inhibitors suitable for the provided compositions and methods include, but are not limited to erlotinib, gefitinib, sapitinib; varlitinib; TAK-285 (N-[2-[4-[3-chloro-4-[3-(trifluoromethyl)phenoxy]phenylamino]-5H-pyrrolo[3,2-d]pyrimidin-5-yl]ethyl]-3-hydroxy-3-methylbutanamide); AEE788 (S)-(6-(4-((4-ethylpiperazin-1-ylmethyl)phenyl]-N-(1-phenylethyl)-7H-pyrrolo[2,3-d]pyrimidin-4-anine); tarloxotinib 3-[N-[4-(3-bromo-4-chlorophenylamino)-pyrido[3,4-d]pyrimidin-6-yl]carbamoyl]-N,N-dimethyl-N-(1-methyl-4-nitro-1H-imidazol-5-ylmethyl)-2(E)-propen-1-aminium bromide); BMS 599626 ((3S)-3-morpholinylmethyl-[4-[[1-|(3-fluorophenyl)methyl]-1H-indazol-5-yl]aminol-5-methylpyrrolo[2,1-f][1,2,4]triazine-6-yl]carbamate dihydrochloride); and GW 583340 (N-[3-chloro-4-(3-fluorobenzyloxy)phenyll-6-[2-[2-(methylsulfonyl)ethylaminomethyl]thiazol-4-yl]quinazolin-4-amine dihydrochloride).

In one embodiment, the pan ErbB family inhibitor is a combination of an EGFR inhibitor and a HER2 inhibitor, wherein the EGFR inhibitor and the HER2 inhibitor are a combination of two of: AG 1478 (N-(3-chlorophenyl)-6,7-dimethoxyquinazolin-4-amine hydrochloride), AG 555 ((E)-2-cyano-3-(3,4-dihydroxyphenyl)-N-(3-phenylpropyl)-2-propenamide); AG 556 ((E)-2-cyano-3-(3,4-dihydroxyphenyl)-N-(4-phenylbutyl)-2-propenamide; AG 825 (E-3-[3-benzothiazol-2-ylsulfanylmethyl)-4-hydroxy-5-methoxyphenyl]-2-cyano-2-propenamide); CP 724714 (2-methoxy-N-1(2E)-3-[4-[3-methyl-4-(6-methylpyridin-3-yloxy)phenylamino]quinazolin-6-yl]-2-propen-1-yl]acetamide; BIBU 1361 (N-(3-chloro-4-fluorophenyl)-6-[4-(diethylaminomethyl)-piperidin-1-yl]pyrimido[5,4-d]pyrimidin-4-amine dihydrochloride); BIBU 1382; (N⁸-(3-chloro-4-fluorophenyl)-N²-(1-methyl-4-piperidinyl)pyrimidino[5,4-d]pyrimidin-4-amine dihydrochloride), JNJ 28871063 (5E-4-amino-6-[4-(benzyloxy)-3-chlorophenylamino]-pyrimidine-5-carbaldehyde N-[2-(4-morpholinyl)ethyl]oxime hydrochloride); PD 153035 (4-(3-bromophenylamino)-6,7-dimethoxyquinazoline hydrochloride); and PD 158780 (N⁴-(3-bromophenyl)-N⁶-methyl-pyrido[3,4-d]pyrimidine-4,6-diamine).

In one embodiment, the pan ErbB family inhibitor is an anti-EGFR antibody, an anti-HER2 antibody or combination of an anti-EGFR antibody and anti-HER2 antibody. Antibodies, including monoclonal antibodies, antibody conjugates and bispecific antibodies, targeting EGFR and/or HER2 are well known and several antibodies are commercially available for research and human clinical use. Examples of anti-EGFR antibodies suitable for the provided compositions and methods include necitumumab, panitumumab and cetuximab. Examples of anti-HER2 antibodies suitable for the provided compositions and methods include, pertuzumab, trastuzumab, and trastuzumab emtansine.

In some embodiments, the compounds of the present disclosure are used in combination with an immune checkpoint inhibitor. Examples of immune checkpoint inhibitors suitable for the provided compositions and methods include, but are not limited to, PD-1, PD-L1, CTLA-4, and LAG-3 inhibitors, such as Pembrolizumab (Keytruda®), Nivolumab (Opdivo®), Cemiplimab (Libtayo®), Atezolizumab (Tecentriq®), Avelumab (Bavencio®), Durvalumab (Imfinzi™), Ipilimumab (Yervoy®), Relatlimab, Opdualag, and Dostarlimab (Jemperli).

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### SELECTED EMBODIMENTS

Embodiment 1. A compound of Formula A, Formula B, or Formula C:

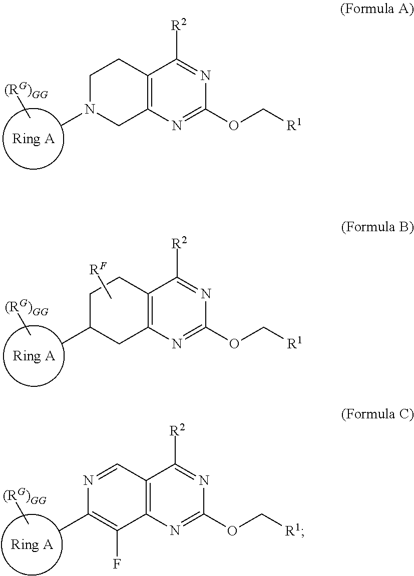

or a salt thereof; and/or an isotopologue thereof; wherein:
Ring A is a 6-membered aryl or a 5-10 membered heteroaryl;
R^{F} is selected from the group consisting of H, halo, C₁-C₄ alkyl, C₁-C₄ alkoxy, C₁-C₄ haloalkyl and C₁-C₄ haloalkoxy;
each R^{G} is independently selected from halo, —OH, —NH₂, C₁-C₄ alkyl, C₁-C₄ alkoxy, C₁-C₄ haloalkyl, C₁-C₄ haloalkoxy, C₃-C₆ cycloalkyl and C₂-C₃ alkynyl;
each GG is independently 0, 1, 2 or 3;
R¹ is a 4-8 membered saturated carbocyclic or heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy;
R² is selected from the group consisting of R^{2b}, R^{2c} and R^{2e};
R^{2b} is —NR¹⁰R¹¹;
R¹⁰ is selected from the group consisting of H, C₁-C₄ alkyl, C₁-C₄ alkoxy, C₁-C₄ haloalkyl, and C₁-C₄ haloalkoxy; and
R¹¹ is —(CH₂)_{w}—R¹³; or
R¹⁰ and R¹¹ together with the nitrogen to which they are attached form a 4-8 membered saturated heterocyclic group comprising a second nitrogen as the sole additional heteroatom within the ring atoms, wherein the second nitrogen of the 4-8 membered saturated heterocyclic group is substituted with cyano, and the 4-8 membered saturated heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
R¹³ is a 4-5 membered saturated heterocyclic group comprising one nitrogen as the sole heteroatom within the ring atoms, wherein the nitrogen is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo; or
R¹³ is a 6 membered saturated heterocyclic group comprising one or two nitrogens as the sole heteroatom(s) within the ring atoms, wherein one of the nitrogens is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom; or
R¹³ is a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom;
w is 0, 1, or 2;
R^{2c} is —NR¹⁵R¹⁶;
R¹⁵ is H, C₁-C₄ alkyl, C₁-C₄ alkoxy, C₁-C₄ haloalkyl, or C₁-C₄ haloalkoxy; and
R¹⁶ is —(CH₂)_{y}—R²¹;
R²¹ is selected from:
a 4-5 membered saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom; or
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom;
R¹⁸ is selected from the group consisting of hydrogen, —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)NR²²R²³, —(CH₂)_{z}—NR²²R²³, —(CH₂)ᵤ—R³⁴, —(C₁-C₂ alkyl)-(C₁-C₂ alkoxy), —S(O)₂—C₁-C₄ alkyl, —S(O)₂—C₁-C₄ haloalkyl, and R³⁵;
R¹⁹ is selected from the group consisting of hydrogen, C₁-C₄ alkyl, C₁-C₄ alkoxy, C₁-C₄ haloalkyl, and C₁-C₄ haloalkoxy;
R²⁰ is selected from the group consisting of hydrogen, C₁-C₄ alkyl, C₁-C₄ alkoxy, C₁-C₄ haloalkyl, and C₁-C₄ haloalkoxy;
R²² and R²³ are independently selected from the group consisting of hydrogen, C₁-C₄ alkyl, C₁-C₄ alkoxy, C₁-C₄ haloalkyl, and C₁-C₄ haloalkoxy;
R³⁴ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl;
R³⁵ is a 5-6 membered heteroaryl group optionally substituted with 0, 1 or 2 substituents independently selected from halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl;
y is 0, 1, or 2;
z is 1 or 2;
q is 0 or 1;
u is 0, 1 or 2;
R^{2e} is —NR²⁸R²⁹;
R²⁸ is H, C₁-C₄ alkyl, C₁-C₄ alkoxy, C₁-C₄ haloalkyl, or C₁-C₄ haloalkoxy; and
R²⁹ is —(CH₂)ₜ—R³⁰;
R³⁰ is selected from:
a 4-5 membered saturated heterocyclic group comprising one nitrogen as the sole heteroatom within the ring atoms, wherein the nitrogen ring atom of the heterocyclic group is substituted with —C(O)C≡CR³¹ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C≡CR³¹, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom; and
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C≡CR³¹, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom;
R³¹ is selected from the group consisting of —(CH₂)ᵥ—NR³²R³³ and —(CH₂)ₚ—R³⁶;
R³² and R³³ are independently selected from the group consisting of hydrogen, C₁-C₄ alkyl, C₁-C₄ alkoxy, C₁-C₄ haloalkyl, and C₁-C₄ haloalkoxy;
t is 0, 1, or 2;
v is 1 or 2;
p is 0, 1 or 2;
   R³⁶ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl;

Embodiment 2. The compound of embodiment 1, wherein the compound is a compound of Formula A, or a salt thereof.

Embodiment 3. The compound of embodiment 1, wherein the compound is a compound of Formula B or Formula C, or a salt thereof.

Embodiment 4. The compound of embodiment 1, wherein the compound is a compound of Formula B, or a salt thereof.

Embodiment 5. The compound of any one of embodiments 1, 3 and 4, wherein the compound of Formula B is of formula B1

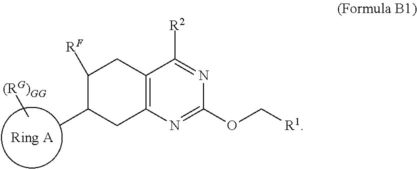

Embodiment 6. The compound of embodiment 1, wherein the compound is a compound of Formula C, or a salt thereof.

Embodiment 7. The compound of any one of embodiments 1, 3, 4 and 5, wherein R^{F} is C₁-C₄ alkyl.

Embodiment 8. The compound of any one of embodiments 1, 3, 4 and 5, wherein R^{F} is methyl.

Embodiment 9. The compound of any one of embodiments 1-8, wherein Ring A is selected from phenyl, pyridinyl and isoquinolinyl.

Embodiment 10. The compound of any one of embodiments 1-8, wherein Ring A is selected from phenyl, pyridin-2-yl, pyridin-4-yl, and isoquinolin-1-yl.

Embodiment 11. The compound of any one of embodiments 1-8, wherein Ring A is selected from phenyl, pyridin-2-yl and isoquinolin-1-yl.

Embodiment 12. The compound of any one of embodiments 1-11, wherein GG is 1, 2 or 3.

Embodiment 13. The compound of any one of embodiments 1-11, wherein GG is 1.

Embodiment 14. The compound of any one of embodiments 1-11 wherein GG is 2 or 3.

Embodiment 15. The compound of any one of embodiments 1-11, wherein GG is 2.

Embodiment 16. The compound of any one of embodiments 1-11, wherein GG is 3.

Embodiment 17. The compound of any one of embodiments 1-8, wherein each moiety represented by

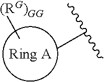

is independently selected from the group consisting of

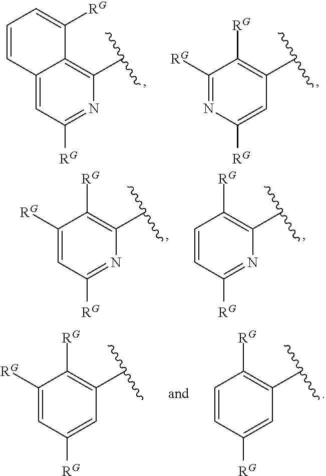

Embodiment 18. The compound of any one of embodiments 1-8, wherein each moiety represented by

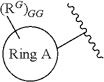

is independently selected from the group consisting of

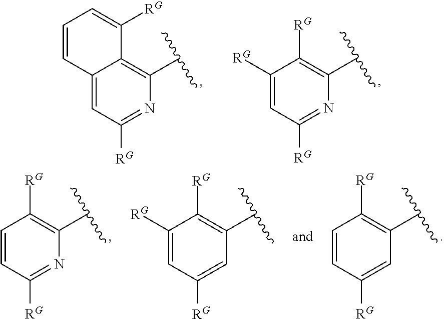

Embodiment 19. The compound of any one of embodiments 1-8, wherein each moiety represented by

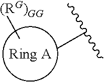

is independently selected from the group consisting of

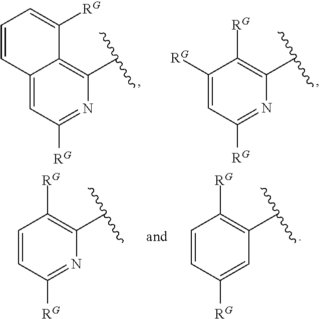

Embodiment 20. The compound of any one of embodiments 1-8, wherein each moiety represented by

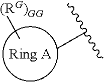

is independently

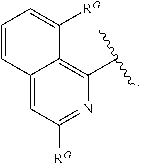

Embodiment 21. The compound of any one of embodiments 1-8, wherein each moiety represented by

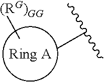

is independently

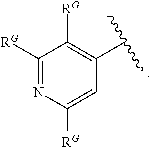

Embodiment 22. The compound of any one of embodiments 1-8, wherein each moiety represented by

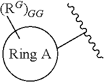

is independently

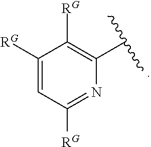

Embodiment 23. The compound of any one of embodiments 1-8, wherein each moiety represented by

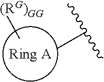

is independently

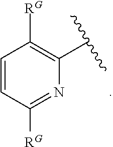

Embodiment 24. The compound of any one of embodiments 1-8, wherein each moiety represented by

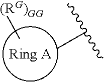

is independently

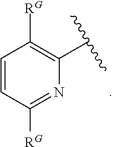

Embodiment 25. The compound of any one of embodiments 1-24, wherein each R^{G} is independently selected from halo, —OH, —NH₂, C₁-C₄ alkyl, C₁-C₄ haloalkyl and C₃-C₆ cycloalkyl.

Embodiment 26. The compound of any one of embodiments 1-24, wherein each R^{G} is independently selected from —F, —Cl, —OH, —NH₂, -Me, —CF₃ and cyclopropyl.

Embodiment 27. The compound of any one of embodiments 1-8, 25 and 26, wherein each moiety represented by

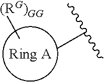

is independently selected from the group consisting of

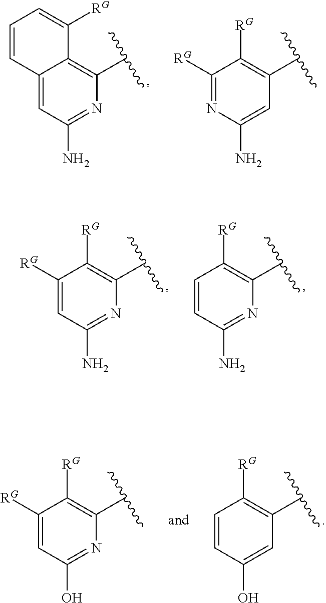

Embodiment 28. The compound of any one of embodiments 1-8, 25 and 26, wherein each moiety represented by

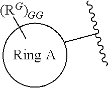

is independently selected from the group consisting of

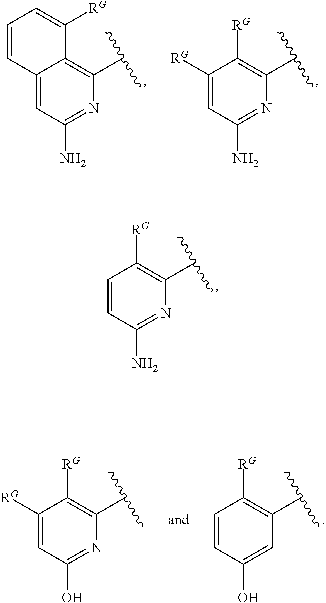

Embodiment 29. The compound of any one of embodiments 1-8, 25 and 26, wherein each moiety represented by

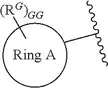

is independently

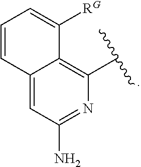

Embodiment 30. The compound of any one of embodiments 1-8, 25 and 26, wherein each moiety represented by

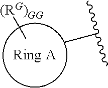

is independently

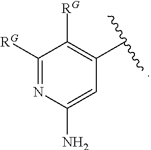

Embodiment 31. The compound of any one of embodiments 1-8, 25 and 26, wherein each moiety represented by

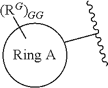

is independently

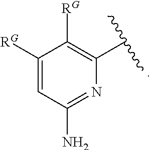

Embodiment 32. The compound of any one of embodiments 1-8, 25 and 26, wherein each moiety represented by

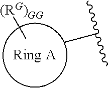

is independently

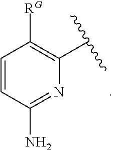

Embodiment 33. The compound of any one of embodiments 1-8, 25 and 26, wherein each moiety represented by

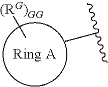

is independently

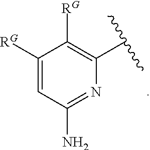

Embodiment 34. The compound of any one of embodiments 1-8, 25 and 26, wherein each moiety represented by

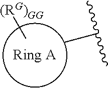

is independently

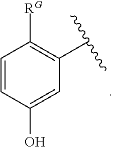

Embodiment 35. The compound of any one of embodiments 1-34, wherein each R^{G} is independently selected from —F, —Cl, -Me, —CF₃ and cyclopropyl.

Embodiment 36. The compound of any one of embodiments 1-8, wherein each moiety represented by

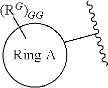

is independently selected from the group consisting of

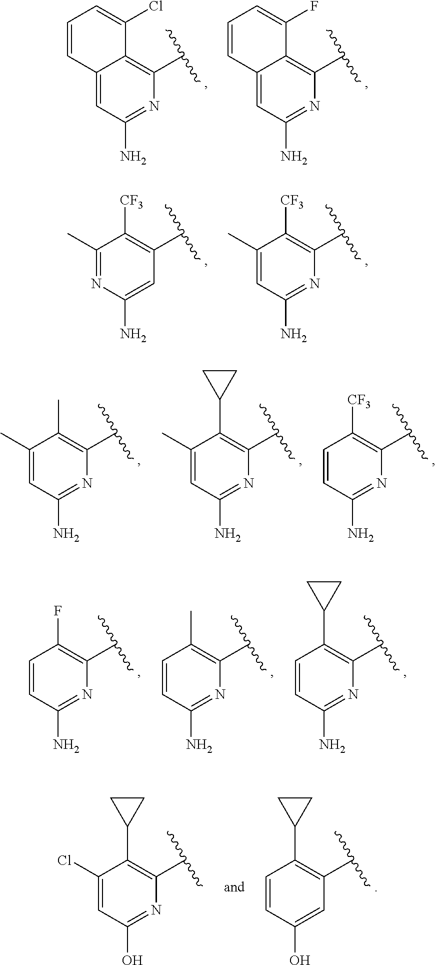

Embodiment 37. The compound of any one of embodiments 1-8, wherein each moiety represented by

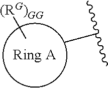

is independently selected from the group consisting of

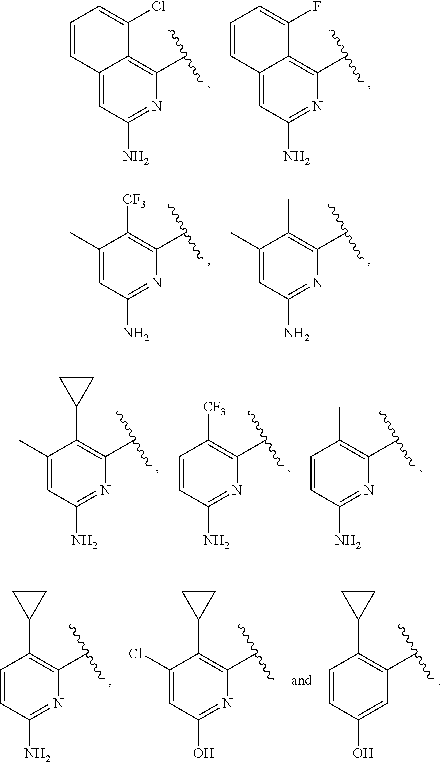

Embodiment 38. The compound of any one of embodiments 1-8, wherein each moiety represented by

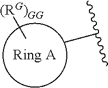

is independently

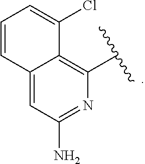 [SMILES: *c1nc(N)cc2cccc(Cl)c12]

Embodiment 39. The compound of any one of embodiments 1-8, wherein each moiety represented by

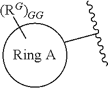

is independently

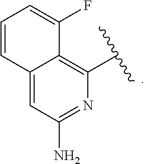 [SMILES: *c1nc(N)cc2cccc(F)c12]

Embodiment 40. The compound of any one of embodiments 1-8, wherein each moiety represented by

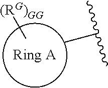

is independently

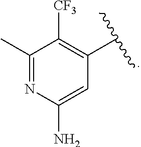 [SMILES: *c1cc(N)nc(C)c1C(F)(F)F]

Embodiment 41. The compound of any one of embodiments 1-8, wherein each moiety represented by

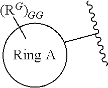

is independently

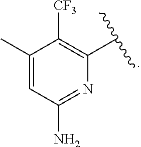 [SMILES: *c1nc(N)cc(C)c1C(F)(F)F]

Embodiment 42. The compound of any one of embodiments 1-8, wherein each moiety represented by

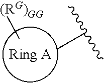

is independently

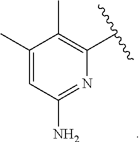 [SMILES: *c1nc(N)cc(C)c1C]

Embodiment 43. The compound of any one of embodiments 1-8, wherein each moiety represented by

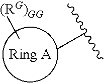

is independently

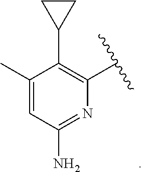 [SMILES: *c1nc(N)cc(C)c1C1CC1]

Embodiment 44. The compound of any one of embodiments 1-8, wherein each moiety represented by

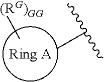

is independently

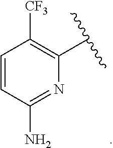 [SMILES: *c1nc(N)ccc1C(F)(F)F]

Embodiment 45. The compound of any one of embodiments 1-8, wherein each moiety represented by

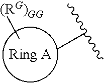

is independently

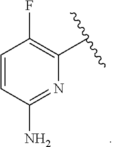 [SMILES: *c1nc(N)ccc1F]

Embodiment 46. The compound of any one of embodiments 1-8, wherein each moiety represented by

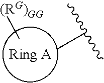

is independently

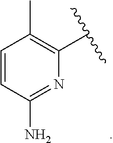 [SMILES: *c1nc(N)ccc1C]

Embodiment 47. The compound of any one of embodiments 1-8, wherein each moiety represented by

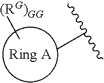

is independently

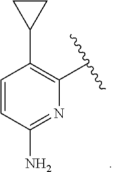 [SMILES: *c1nc(N)ccc1C1CC1]

Embodiment 48. The compound of any one of embodiments 1-8, wherein each moiety represented by

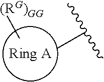

is independently

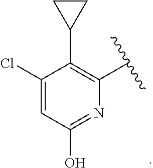 [SMILES: *c1nc(O)cc(Cl)c1C1CC1]

Embodiment 49. The compound of any one of embodiments 1-8, wherein each moiety represented by

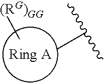

is independently

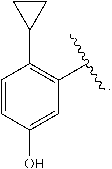 [SMILES: *c1cc(O)ccc1C1CC1]

Embodiment 50. The compound of any one of embodiments 1-49, wherein R¹ is a 4-8 membered saturated monocyclic carbocyclic or monocyclic heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

Embodiment 51. The compound of any one of embodiments 1-49, wherein R¹ is a 4-8 membered saturated bicyclic carbocyclic or bicyclic heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

Embodiment 52. The compound of any one of embodiments 1-51, wherein R¹ is a 4-8 membered saturated heterocyclic group comprising one nitrogen as the sole heteroatom within the ring atoms, wherein the heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

Embodiment 53. The compound of any one of embodiments 1-51, wherein R¹ is a 4-8 membered saturated carbocyclic group substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

Embodiment 54. The compound of any one of embodiments 1-53, wherein R¹ is an unsubstituted 4-8 membered saturated carbocyclic or heterocyclic group comprising one nitrogen as the sole heteroatom within the ring atoms.

Embodiment 55. The compound of any one of embodiments 1-53, wherein the carbocyclic or heterocyclic group of R¹ is unsubstituted, or is substituted with one halo, hydroxy or C₁-C₄ alkyl.

Embodiment 56. The compound of any one of embodiments 1-53, wherein the carbocyclic or heterocyclic group of R¹ is unsubstituted, or is substituted with one halo or hydroxy.

Embodiment 57. The compound of any one of embodiments 1-53, wherein the carbocyclic or heterocyclic group of R¹ is unsubstituted, or is substituted with one fluoro.

Embodiment 58. The compound of any one of embodiments 1-53, wherein the carbocyclic or heterocyclic group of R¹ is substituted with one fluoro.

Embodiment 59. The compound of any one of embodiments 1-53, wherein the carbocyclic or heterocyclic group of R¹ is substituted with one C₁-C₄ alkyl.

Embodiment 60. The compound of any one of embodiments 1-49, wherein R¹ is selected from the group consisting of:

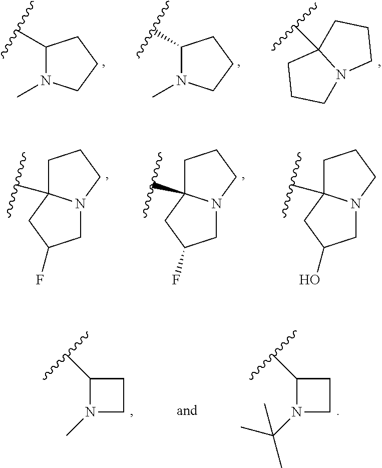

Embodiment 61. The compound of any one of embodiments 1-49, wherein R¹ is selected from the group consisting of:

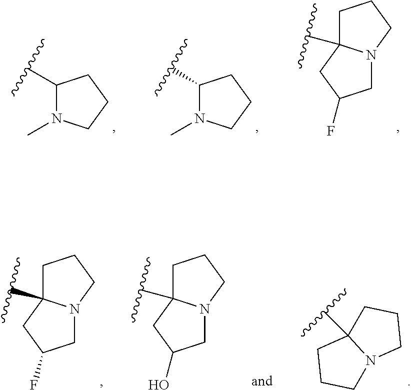

Embodiment 62. The compound of any one of embodiments 1-49, wherein R¹ is selected from the group consisting of:

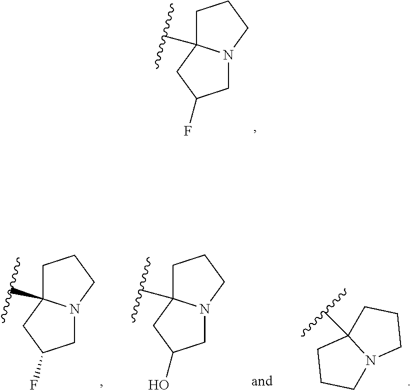 [SMILES: *C12CCCN1CC(F)C2 ; *[C@@]12CCCN1C[C@H](F)C2 ; *C12CCCN1CC(O)C2 ; *C12CCCN1CCC2]

Embodiment 63. The compound of any one of embodiments 1-49, wherein R¹ is selected from the group consisting of:

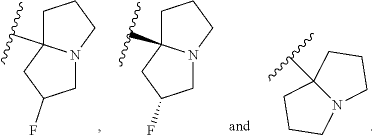

Embodiment 64. The compound of any one of embodiments 1-49, wherein R¹ is

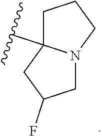 [SMILES: *C12CCCN1CC(F)C2]

Embodiment 65. The compound of any one of embodiments 1-49, wherein R¹ is

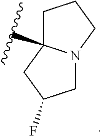 [SMILES: *[C@@]12CCCN1C[C@H](F)C2]

Embodiment 66. The compound of any one of embodiments 1-50, wherein R¹ is

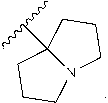 [SMILES: *C12CCCN1CCC2]

Embodiment 67. The compound of any one of embodiments 1-66, wherein R² is selected from the group consisting of R^{2b} and R^{2c}.

Embodiment 68. The compound of any one of embodiments 1-66, wherein R² is selected from the group consisting of R^{2b} and R^{2e}.

Embodiment 69. The compound of any one of embodiments 1-66, wherein R² is selected from the group consisting of R^{2c} and R^{2e}.

Embodiment 70. The compound of any one of embodiments 1-66, wherein R² is R^{2b}.

Embodiment 71. The compound of any one of embodiments 1-68 and 70, wherein R¹⁰ is methyl or ethyl.

Embodiment 72. The compound of any one of embodiments 1-68 and 70, wherein R¹⁰ is methyl.

Embodiment 73. The compound of any one of embodiments 1-68 and 70-72, wherein R¹¹ is —(CH₂)_{w}—R¹³.

Embodiment 74. The compound of any one of embodiments 1-68 and 70-73, wherein w is 0 or 1.

Embodiment 75. The compound of any one of embodiments 1-68 and 70-74, wherein R¹³ is a 4-7 membered saturated heterocyclic group comprising one nitrogen as the sole heteroatom within the ring atoms, wherein the nitrogen is substituted with cyano, and wherein the heterocyclic group is optionally further substituted with 1 substituent selected from the group consisting of hydroxy, CN, C₁-C₄ cyanoalkyl, C₁-C₄ alkyl, C₁-C₄ alkoxy, or halo.

Embodiment 76. The compound of any one of embodiments 1-68 and 70-75, wherein the heterocyclic group of R¹³ is not further substituted.

Embodiment 77. The compound of any one of embodiments 1-68 and 70-75, wherein the heterocyclic group of R¹³ is further substituted with 1 substituent selected from the group consisting of hydroxy, CN, C₁-C₄ cyanoalkyl, C₁-C₄ alkyl, C₁-C₄ alkoxy, or halo.

Embodiment 78. The compound of any one of embodiments 1-68 and 70-75, wherein the heterocyclic group of R¹³ is further substituted with 1 substituent selected from the group consisting of C₁-C₄ alkyl, C₁-C₄ alkoxy, or halo.

Embodiment 79. The compound of any one of embodiments 1-68 and 70-75, wherein the heterocyclic group of R¹³ is further substituted with methyl, methoxy, or fluoro.

Embodiment 80. The compound of any one of embodiments 1-68 and 70-73, wherein R¹¹ is selected from the group consisting of:

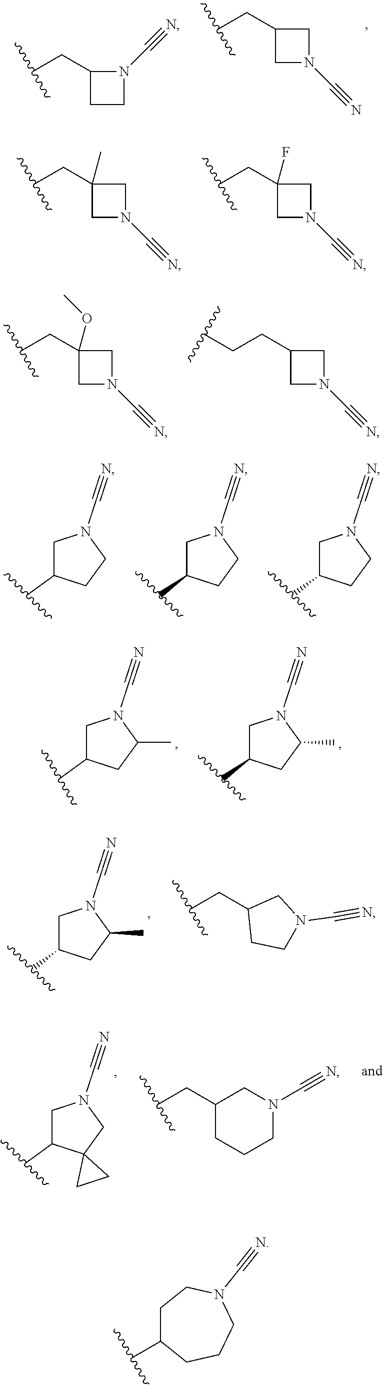

Embodiment 81. The compound of any one of embodiments 1-68 and 70-73, wherein R¹¹ is selected from the group consisting of:

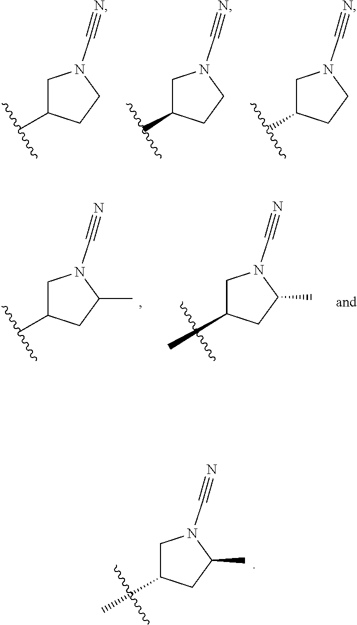

Embodiment 82. The compound of any one of embodiments 1-68 and 70, wherein R¹⁰ and R¹¹ together with the nitrogen to which they are attached form a 4-8 membered saturated heterocyclic group comprising a second nitrogen as the sole additional heteroatom within the ring atoms, wherein the second nitrogen of the 4-8 membered saturated heterocyclic group is substituted with cyano, and the 4-8 membered saturated heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 83. The compound of any one of embodiments 1-68 and 70, wherein the 4-8 membered saturated heterocyclic group formed by R¹⁰ and R¹¹ together with the nitrogen to which they are attached is selected from the group consisting of:

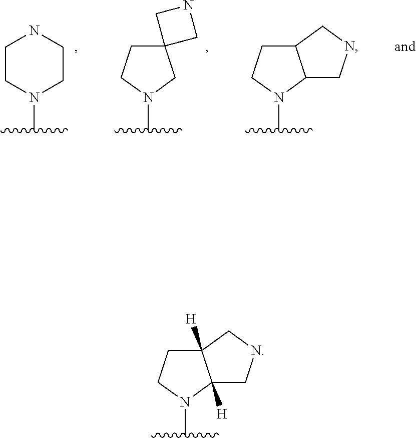

wherein the second nitrogen atom is substituted with cyano and the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 84. The compound of any one of embodiments 1-68 and 70, wherein the 4-8 membered saturated heterocyclic group formed by R¹⁰ and R¹¹ together with the nitrogen to which they are attached is

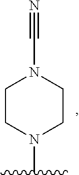 [SMILES: *N1CCN(C#N)CC1]

optionally further substituted with 1 instance of —CH₂CN.

Embodiment 85. The compound of any one of embodiments 1-66, wherein R² is R^{2e}.

Embodiment 86. The compound of any one of embodiments 1-67, 69 and 71-85, wherein R¹⁵ is selected from the group consisting of C₁-C₄ alkyl and C₁-C₄ alkoxy.

Embodiment 87. The compound of any one of embodiments 1-67, 69 and 71-85, wherein R¹⁵ is selected from the group consisting of methyl, ethyl, and —CH₂CH₂OCH₃.

Embodiment 88. The compound of any one of embodiments 1-67, 69 and 71-85, wherein R¹⁵ is methyl or ethyl.

Embodiment 89. The compound of any one of embodiments 1-67, 69 and 71-85, wherein R¹⁵ is methyl.

Embodiment 90. The compound of any one of embodiments 1-67, 69 and 71-89, wherein y is 0 or 1.

Embodiment 91. The compound of any one of embodiments 1-67, 69 and 71-89, wherein y is 0.

Embodiment 92. The compound of any one of embodiments 1-67, 69 and 71-89, wherein y is 1.

Embodiment 93. The compound of any one of embodiments 1-67, 69 and 71-92 wherein R²¹ is selected from:
a 4-5 membered saturated monocyclic heterocyclic group comprising one nitrogen as the sole heteroatom within the ring atoms, wherein the nitrogen ring atom is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated monocyclic heterocyclic group comprising one or two nitrogens as the sole heteroatom(s) within the ring atoms, wherein one of the nitrogens is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom; and
a 7 membered saturated monocyclic heterocyclic group comprising one nitrogen, and optionally one additional heteroatom selected from nitrogen, oxygen, and sulfur, as the sole heteroatom(s) within the ring atoms, wherein one of the nitrogen ring atom(s) is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom.

Embodiment 94. The compound of any one of embodiments 1-67, 69 and 71-92, wherein R²¹ is selected from:
a 4-5 membered monocyclic saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered monocyclic saturated heterocyclic group comprising one or two nitrogens as the sole heteroatom(s) within the ring atoms, wherein one of the nitrogens of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom; and
a 7 membered saturated bicyclic spirocyclic heterocyclic group comprising one nitrogen as the sole heteroatom within the ring atoms, wherein the nitrogen ring atom is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom.

Embodiment 95. The compound of any one of embodiments 1-67, 69 and 71-92, wherein R²¹ is a 4-5 membered monocyclic saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 96. The compound of any one of embodiments 1-67, 69 and 71-95 wherein the heterocyclic group of R²¹ is not further substituted.

Embodiment 97. The compound of any one of embodiments 1-67, 69 and 71-95 wherein the heterocyclic group of R²¹ is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 98. The compound of any one of embodiments 1-67, 69 and 71-95 wherein the heterocyclic group of R²¹ is further substituted with 1 substituent selected from the group consisting of hydroxy, CN, C₁-C₄ alkyl, C₁-C₄ cyanoalkyl, and halo.

Embodiment 99. The compound of any one of embodiments 1-67, 69 and 71-95 wherein the heterocyclic group of R²¹ is further substituted with 1 substituent selected from the group consisting of hydroxy, CN, Me, —CH₂CN and F.

Embodiment 100. The compound of any one of embodiments 1-67, 69 and 71-92, wherein the heterocyclic group of R²¹ is selected from the group consisting of:

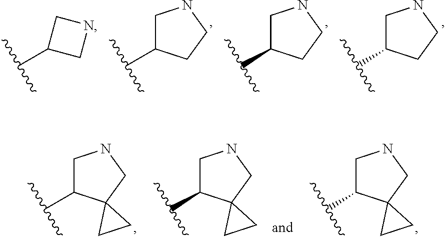

wherein the ring nitrogen of the heterocyclic group is substituted with —C(O)C(R¹⁹)═C(R²⁰)R¹⁸ and the heterocyclic group is not further substituted, or is substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 101. The compound of any one of embodiments 1-67, 69 and 71-92, wherein the heterocyclic group of R²¹ is selected from the group consisting of:

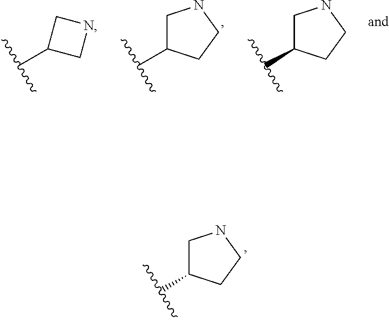

wherein the ring nitrogen of the heterocyclic group is substituted with —C(O)C(R¹⁹)═C(R²⁰)R¹⁸ and the heterocyclic group is not further substituted, or is substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 102. The compound of embodiment 100 or 101, wherein the heterocyclic group of R²¹ is not further substituted.

Embodiment 103. The compound of embodiment 100 or 101, wherein the heterocyclic group of R²¹ is further substituted with 1 substituent selected from the group consisting of hydroxy, CN, C₁-C₄ alkyl, C₁-C₄ cyanoalkyl, and halo.

Embodiment 104. The compound of embodiment 100 or 101, wherein the heterocyclic group of R²¹ is further substituted with 1 substituent selected from the group consisting of hydroxy, CN, Me, —CH₂CN and F.

Embodiment 105. The compound of embodiment 100 or 101, wherein the heterocyclic group of R²¹ is selected from the group consisting of:

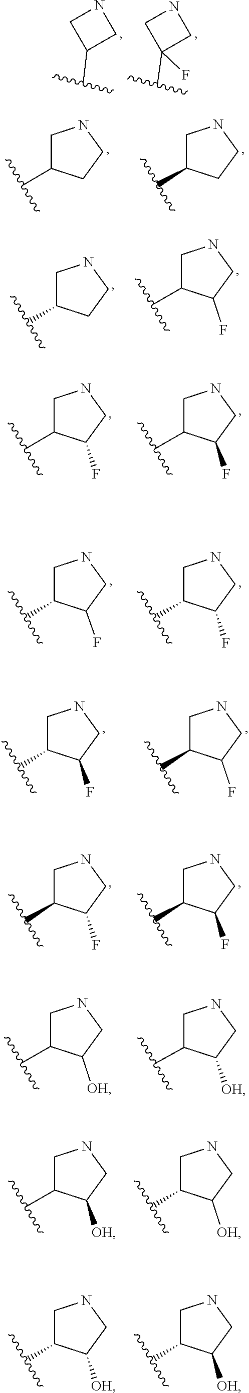

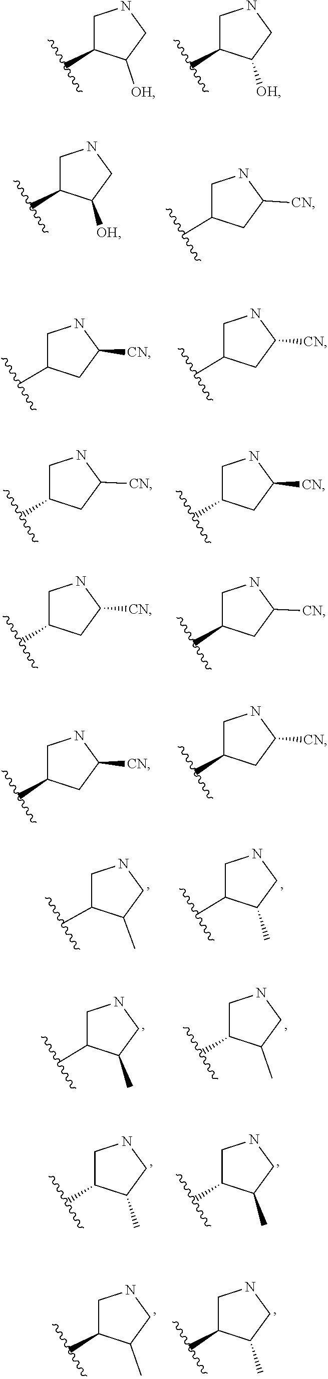

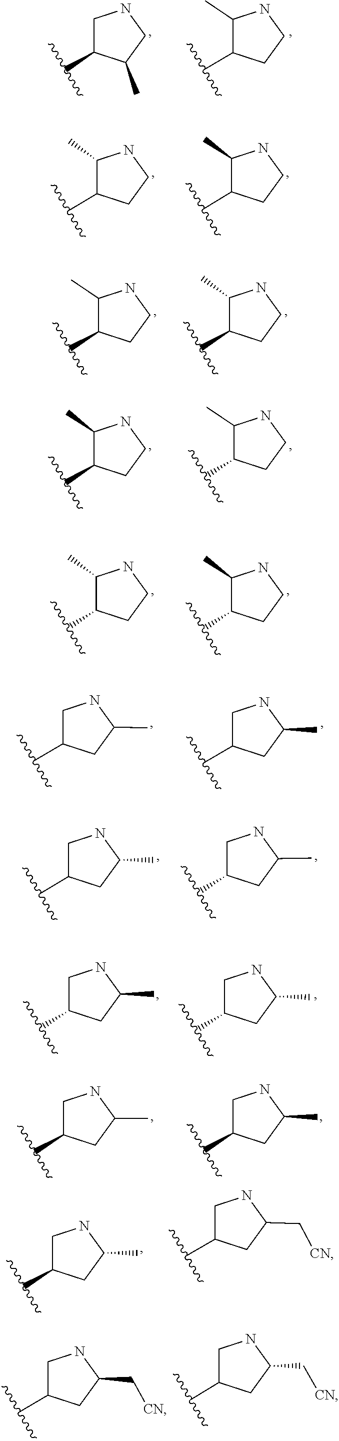

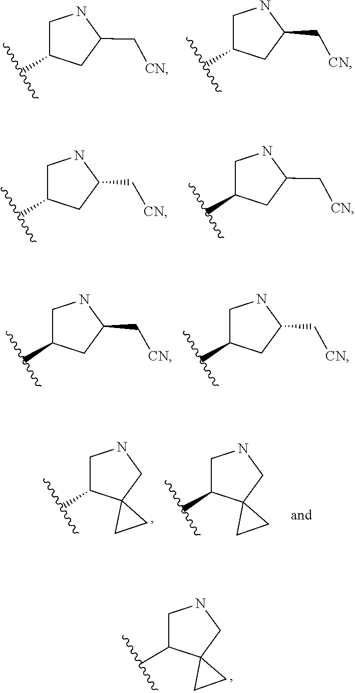

Embodiment 106. The compound of embodiment 100 or 101, wherein the heterocyclic group of R²¹ is selected from the group consisting of:

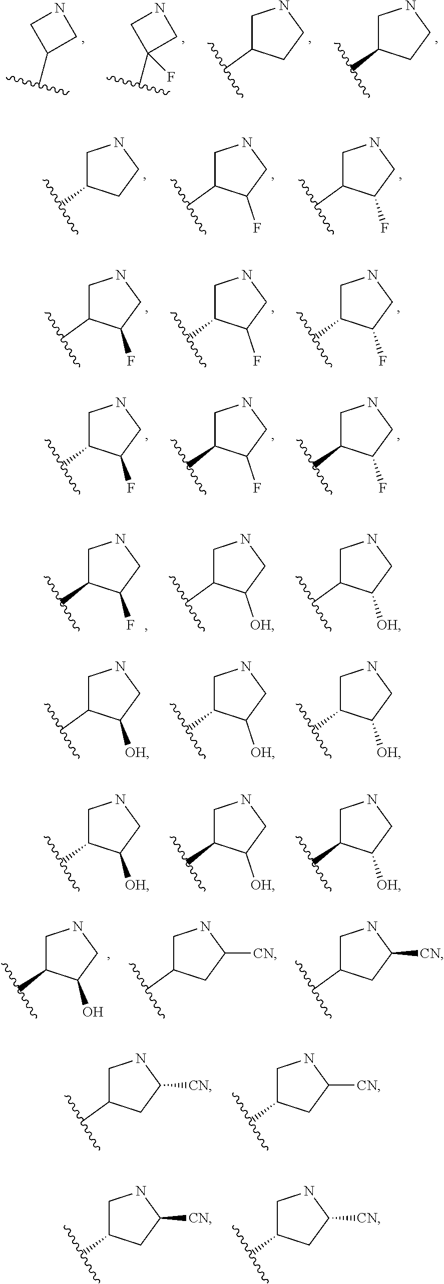

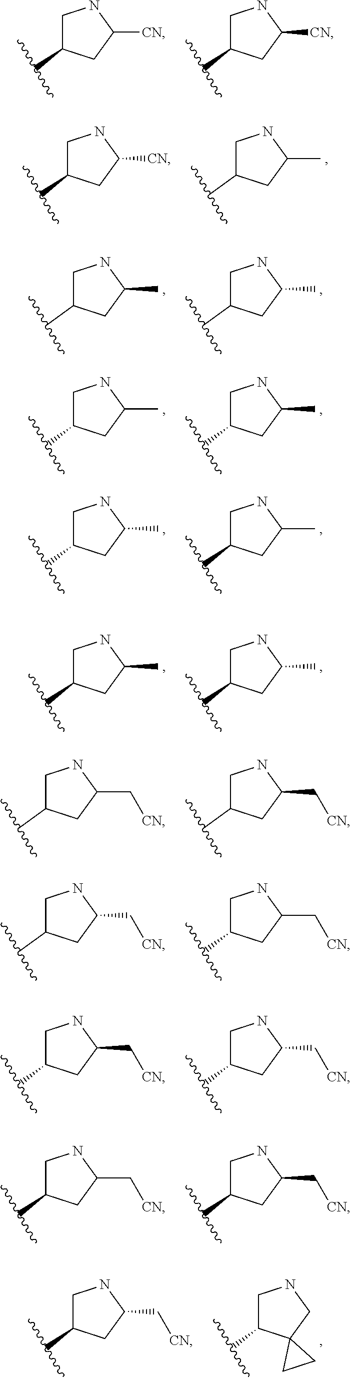

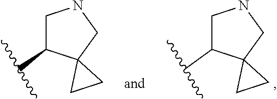 [SMILES: *[C@H]1CNCC12CC2 ; *C1CNCC12CC2]

wherein the ring nitrogen of the heterocyclic group is substituted with —C(O)C(R¹⁹)═C(R²⁰)R¹⁸.

Embodiment 107. The compound of embodiment 101, wherein the heterocyclic group of R²¹ is selected from the group consisting of:

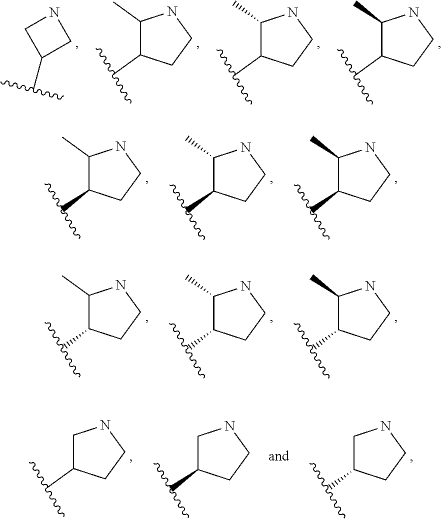

wherein the ring nitrogen of the heterocyclic group is substituted with —C(O)C(R¹⁹)═C(R²⁰)R¹⁸.

Embodiment 108. The compound of embodiment 101, wherein the heterocyclic group of R²¹ is

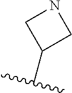 [SMILES: *C1CNC1]

wherein the ring nitrogen of the heterocyclic group is substituted with —C(O)C(R¹⁹)═C(R²⁰)R¹⁸.

Embodiment 109. The compound of embodiment 101, wherein the heterocyclic group of R²¹ is

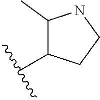 [SMILES: *C1CCNC1C]

wherein the ring nitrogen of the heterocyclic group is substituted with —C(O)C(R¹⁹)═C(R²⁰)R¹⁸.

Embodiment 110. The compound of embodiment 101, wherein the heterocyclic group of R²¹ is

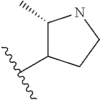 [SMILES: *C1CCN[C@H]1C]

wherein the ring nitrogen of the heterocyclic group is substituted with —C(O)C(R¹⁹)═C(R²⁰)R¹⁸.

Embodiment 111. The compound of embodiment 101, wherein the heterocyclic group of R²¹ is

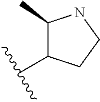 [SMILES: *C1CCN[C@@H]1C]

wherein the ring nitrogen of the heterocyclic group is substituted with —C(O)C(R¹⁹)═C(R²⁰)R¹⁸.

Embodiment 112. The compound of embodiment 101, wherein the heterocyclic group of R²¹ is

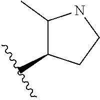 [SMILES: *[C@@H]1CCNC1C]

wherein the ring nitrogen of the heterocyclic group is substituted with —C(O)C(R¹⁹)═C(R²⁰)R¹⁸.

Embodiment 113. The compound of embodiment 101, wherein the heterocyclic group of R²¹ is

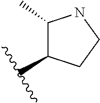 [SMILES: *[C@@H]1CCN[C@H]1C]

wherein the ring nitrogen of the heterocyclic group is substituted with —C(O)C(R¹⁹)═C(R²⁰)R¹⁸.

Embodiment 114. The compound of embodiment 101, wherein the heterocyclic group of R²¹ is

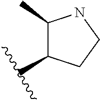 [SMILES: *[C@@H]1CCN[C@@H]1C]

wherein the ring nitrogen of the heterocyclic group is substituted with —C(O)C(R¹⁹)═C(R²⁰)R¹⁸.

Embodiment 115. The compound of embodiment 101, wherein the heterocyclic group of R²¹ is

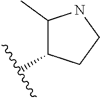 [SMILES: *[C@H]1CCNC1C]

wherein the ring nitrogen of the heterocyclic group is substituted with —C(O)C(R¹⁹)═C(R²⁰)R¹⁸.

Embodiment 116. The compound of embodiment 101, wherein the heterocyclic group of R²¹ is

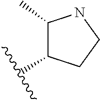 [SMILES: *[C@H]1CCN[C@H]1C]

wherein the ring nitrogen of the heterocyclic group is substituted with —C(O)C(R¹⁹)═C(R²⁰)R¹⁸.

Embodiment 117. The compound of embodiment 101, wherein the heterocyclic group of R²¹ is

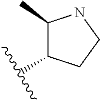 [SMILES: *[C@H]1CCN[C@@H]1C]

wherein the ring nitrogen of the heterocyclic group is substituted with —C(O)C(R¹⁹)═C(R²⁰)R¹⁸.

Embodiment 118. The compound of embodiment 101, wherein the heterocyclic group of R²¹ is

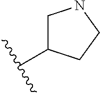 [SMILES: *C1CCNC1]

wherein the ring nitrogen of the heterocyclic group is substituted with —C(O)C(R¹⁹)═C(R²⁰)R¹⁸.

Embodiment 119. The compound of embodiment 101, wherein the heterocyclic group of R²¹ is

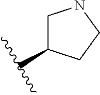 [SMILES: *[C@@H]1CCNC1]

wherein the ring nitrogen of the heterocyclic group is substituted with —C(O)C(R¹⁹)═C(R²⁰)R¹⁸.

Embodiment 120. The compound of embodiment 101, wherein the heterocyclic group of R²¹ is

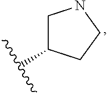 [SMILES: *[C@H]1CCNC1]

wherein the ring nitrogen of the heterocyclic group is substituted with —C(O)C(R¹⁹)═C(R²⁰)R¹⁸.

Embodiment 121. The compound of embodiment 101, wherein the heterocyclic group of R²¹ is selected from the group consisting of:

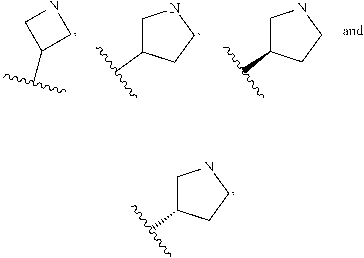 [SMILES: *C1CNC1 ; *C1CCNC1 ; *[C@@H]1CCNC1 ; *[C@H]1CCNC1]

wherein the ring nitrogen of the heterocyclic group is substituted with —C(O)C(R¹⁹)═C(R²⁰)R¹⁸.

Embodiment 122. The compound of any one of embodiments 1-67, 69 and 71-89, wherein R¹⁶ is selected from the group consisting of:

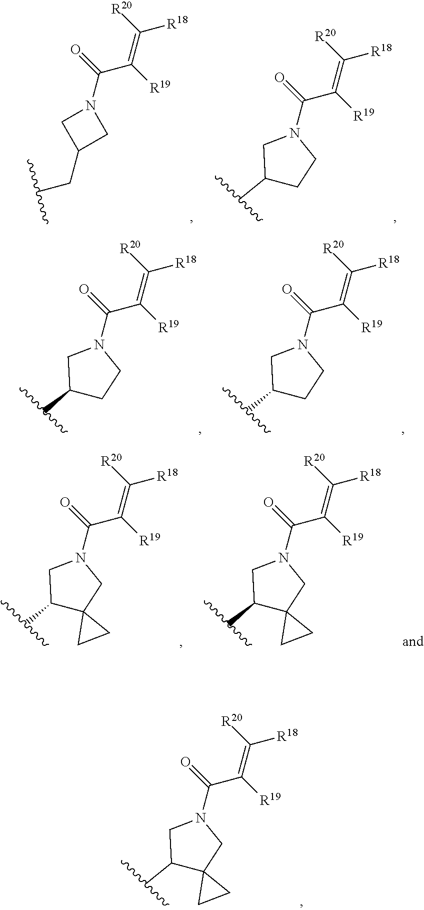

wherein the azetidine, pyrrolidine and 5-azaspiro[2.4]heptane groups are not further substituted, or are substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 123. The compound of any one of embodiments 1-67, 69 and 71-89, wherein R¹⁶ is selected from the group consisting of:

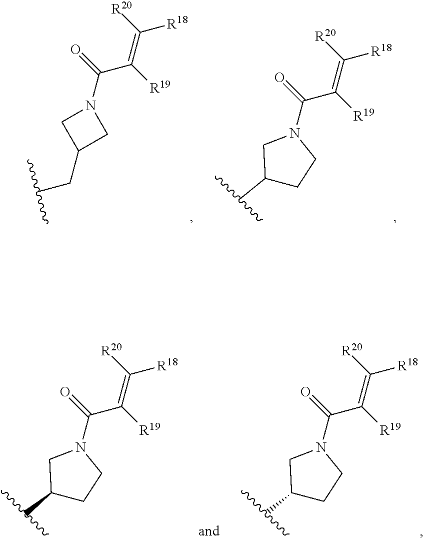

wherein the azetidine, pyrrolidine and 5-azaspiro[2.4]heptane groups are not further substituted, or are substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 124. The compound of embodiment 122 or 123, wherein the azetidine, pyrrolidine and 5-azaspiro[2.4]heptane groups are not further substituted.

Embodiment 125. The compound of embodiment 122 or 123, wherein the azetidine, pyrrolidine and 5-azaspiro[2.4]heptane groups are further substituted with 1 substituent selected from the group consisting of hydroxy, CN, C₁-C₄ alkyl, C₁-C₄ cyanoalkyl, and halo.

Embodiment 126. The compound of embodiment 122 or 123, wherein the azetidine, pyrrolidine and 5-azaspiro[2.4]heptane groups are further substituted with 1 substituent selected from the group consisting of hydroxy, CN, Me, —CH₂CN and F.

Embodiment 127. The compound of any one of embodiments 1-67, 69 and 71-89, wherein R¹⁶ is selected from the group consisting of:

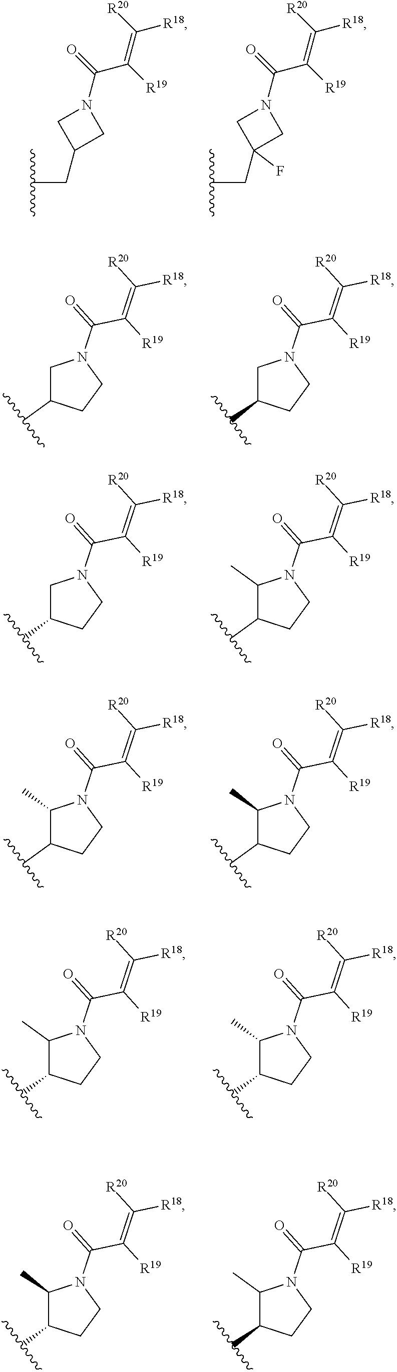

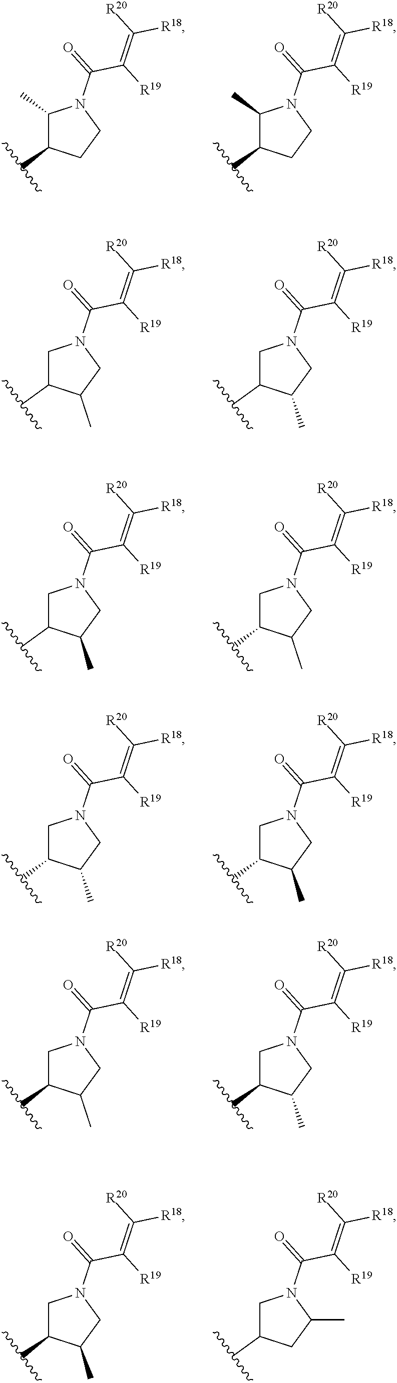

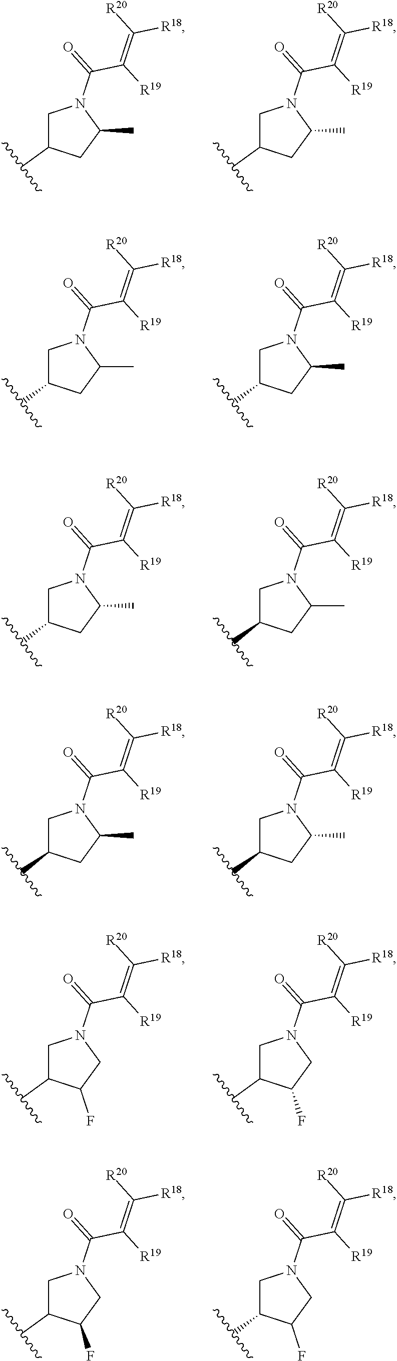

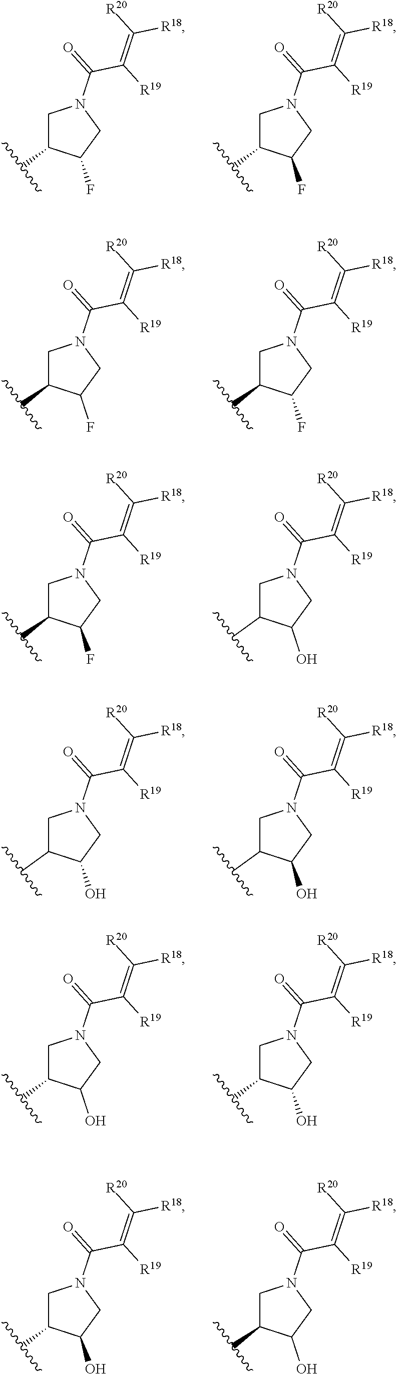

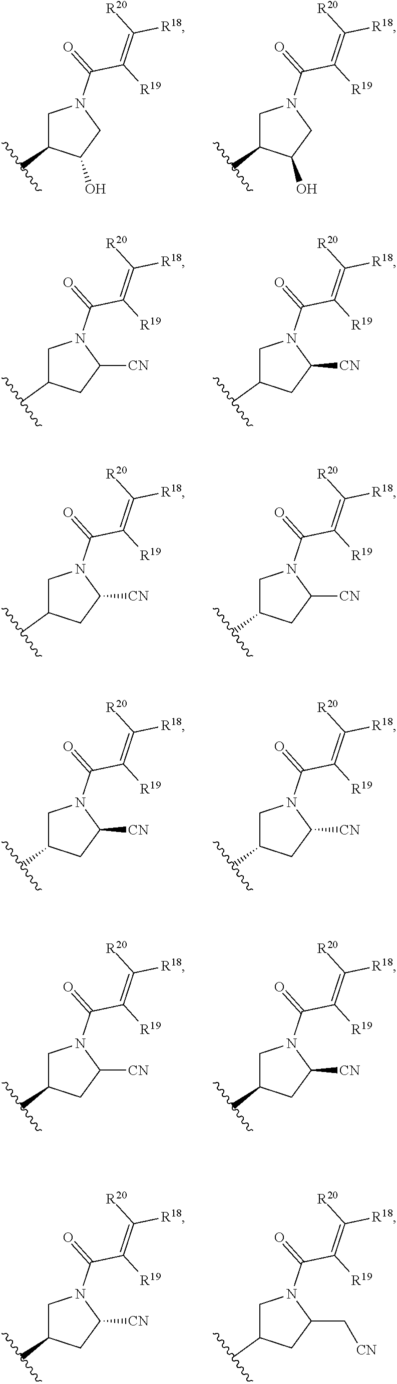

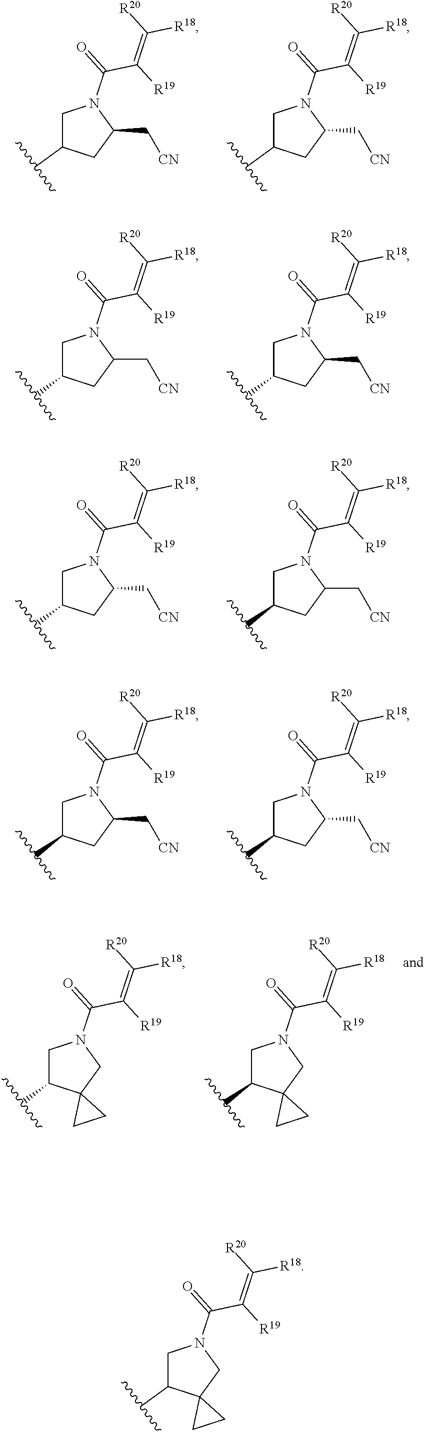

Embodiment 128. The compound of any one of embodiments 1-67, 69 and 71-89, wherein R¹⁶ is selected from the group consisting of:

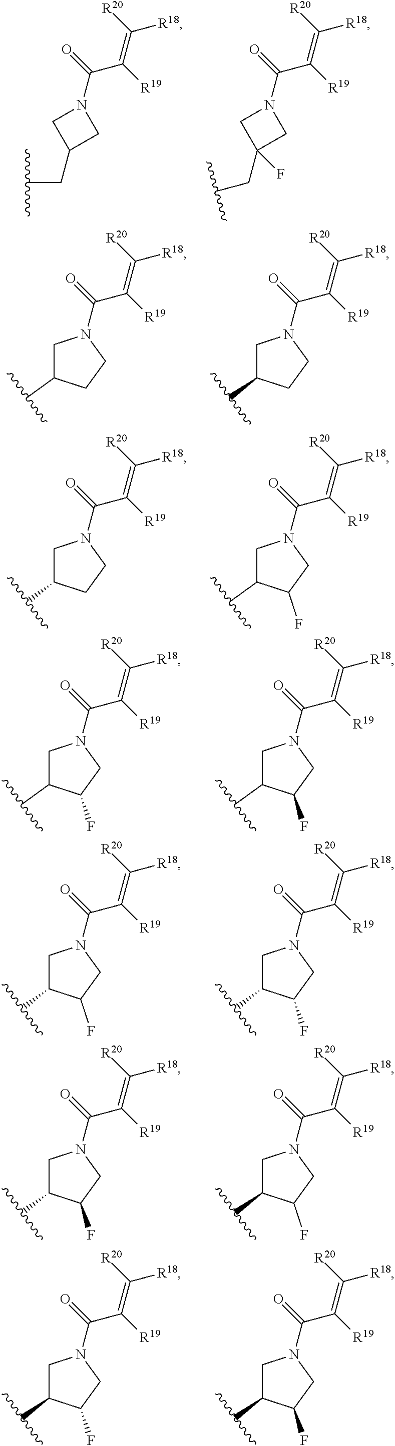

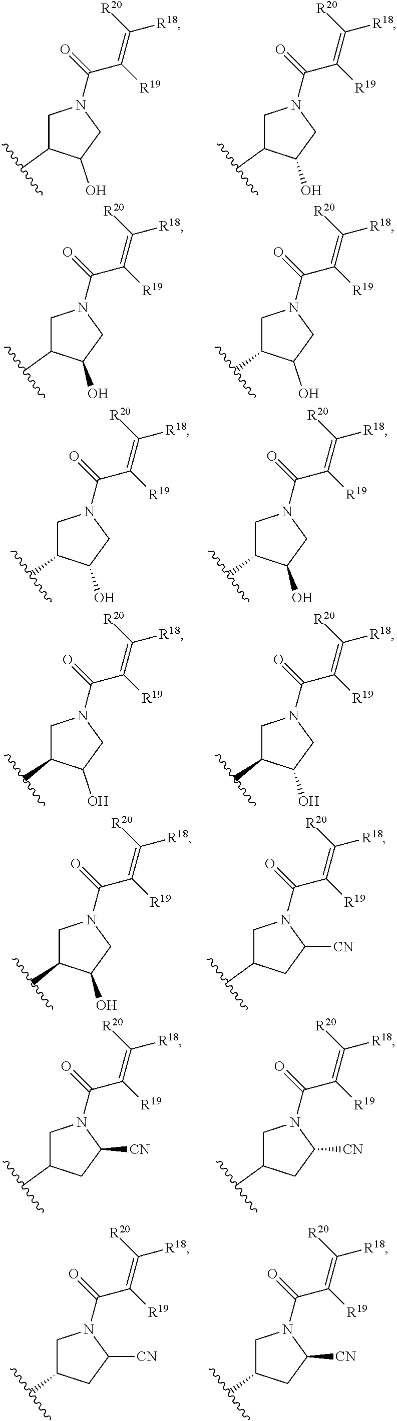

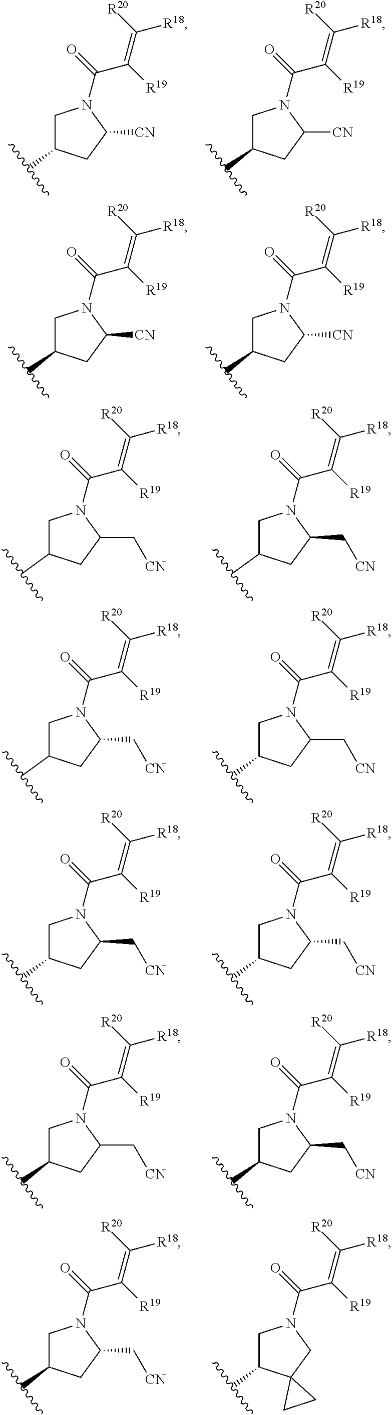

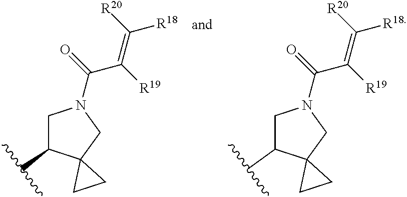

Embodiment 129. The compound of any one of embodiments 1-67, 69 and 71-89, wherein R¹⁶ is selected from the group consisting of:

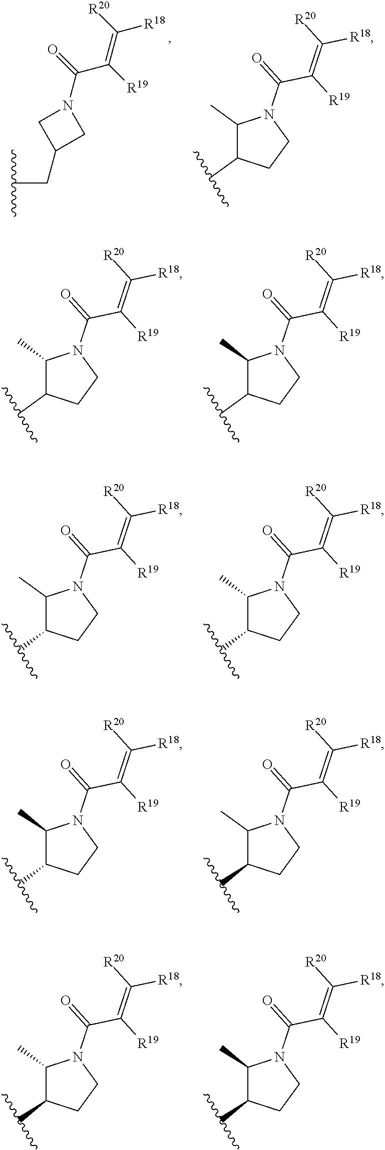

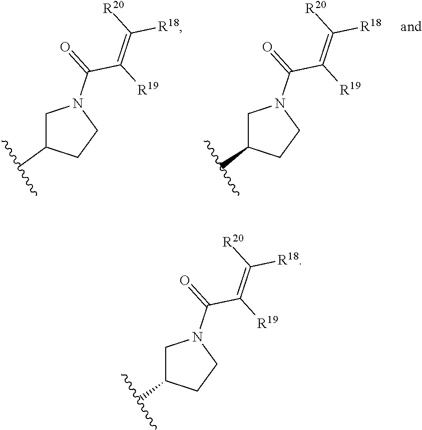

Embodiment 130. The compound of any one of embodiments 1-67, 69 and 71-89, wherein R¹⁶ is

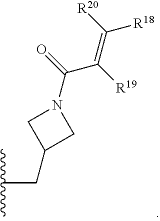

Embodiment 131. The compound of any one of embodiments 1-67, 69 and 71-89, wherein R¹⁶ is

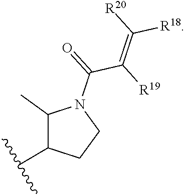

Embodiment 132. The compound of any one of embodiments 1-67, 69 and 71-89, wherein R¹⁶ is

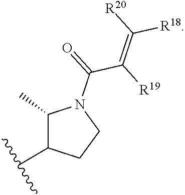

Embodiment 133. The compound of any one of embodiments 1-67, 69 and 71-89, wherein R¹⁶ is

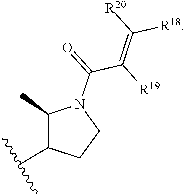

Embodiment 134. The compound of any one of embodiments 1-67, 69 and 71-89, wherein R¹⁶ is

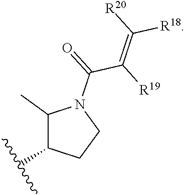

Embodiment 135. The compound of any one of embodiments 1-67, 69 and 71-89, wherein R¹⁶ is

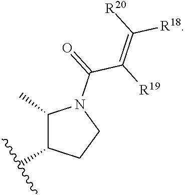

Embodiment 136. The compound of any one of embodiments 1-67, 69 and 71-89, wherein R¹⁶ is

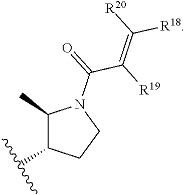

Embodiment 137. The compound of any one of embodiments 1-67, 69 and 71-89, wherein R¹⁶ is

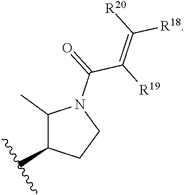

Embodiment 138. The compound of any one of embodiments 1-67, 69 and 71-89, wherein R¹⁶ is

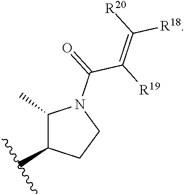

Embodiment 139. The compound of any one of embodiments 1-67, 69 and 71-89, wherein R¹⁶ is

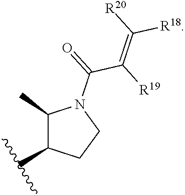

Embodiment 140. The compound of any one of embodiments 1-67, 69 and 71-89, wherein R¹⁶ is

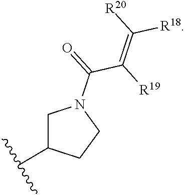

Embodiment 141. The compound of any one of embodiments 1-67, 69 and 71-89, wherein R¹⁶ is

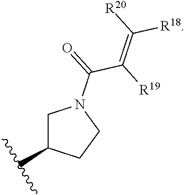

Embodiment 142. The compound of any one of embodiments 1-67, 69 and 71-89, wherein R¹⁶ is

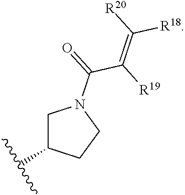

Embodiment 143. The compound of any one of embodiments 1-67, 69 and 71-89, wherein R¹⁶ is selected from the group consisting of:

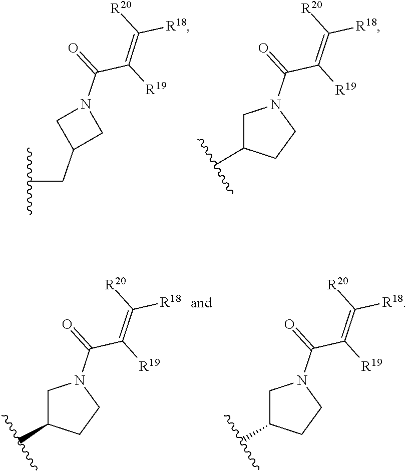

Embodiment 144. The compound of any one of embodiments 1-67, 69 and 71-143, wherein R¹⁹ is hydrogen.

Embodiment 145. The compound of any one of embodiments 1-67, 69 and 71-144, wherein R²⁰ is selected from the group consisting of hydrogen and methyl.

Embodiment 146. The compound of any one of embodiments 1-67, 69 and 71-144, wherein R²⁰ is hydrogen.

Embodiment 147. The compound of any one of embodiments 1-67, 69 and 71-146, wherein R¹⁸ is selected from the group consisting of hydrogen, —COOH, —C(O)O—C₁-C₄ alkyl, —C(O)—C₁-C₄ alkyl, —C(O)NR²²R²³, —(CH₂)_{z}—NR²²R²³, —(CH₂)ᵤ—R³⁴, —(C₁-C₂ alkyl)-(C₁-C₂ alkoxy), —S(O)₂—C₁-C₄ alkyl, and R.

Embodiment 148. The compound of any one of embodiments 1-67, 69 and 71-146, wherein R¹⁸ is selected from the group consisting of hydrogen, —(CH₂)_{z}—NR²²R²³ and —(CH₂)ᵤ—R³⁴.

Embodiment 149. The compound of any one of embodiments 1-67, 69 and 71-148, wherein R²² and R²³ are independently selected from methyl and ethyl.

Embodiment 150. The compound of any one of embodiments 1-67, 69 and 71-146, wherein R¹⁸ is selected from the group consisting of H and —(CH₂)ᵤ—R³⁴.

Embodiment 151. The compound of any one of embodiments 1-67, 69 and 71-149, wherein z is 1 or 2.

Embodiment 152. The compound of any one of embodiments 1-67, 69 and 71-149, wherein z is 1.

Embodiment 153. The compound of any one of embodiments 1-67, 69 and 71-149, wherein z is 2.

Embodiment 154. The compound of any one of embodiments 1-67, 69 and 71-153, wherein u is 0 or 1.

Embodiment 155. The compound of any one of embodiments 1-67, 69 and 71-153, wherein u is 0.

Embodiment 156. The compound of any one of embodiments 1-67, 69 and 71-153, wherein u is 1.

Embodiment 157. The compound of any one of embodiments 1-67, 69 and 71-153, wherein u is 2.

Embodiment 158. The compound of any one of embodiments 1-67, 69 and 71-149, wherein R¹⁸ is selected from H, —CH₂—NR²²R²³, —R³⁴, —CH₂—R³⁴ and —R³⁵.

Embodiment 159. The compound of any one of embodiments 1-67, 69 and 71-153, wherein R¹⁸ is selected from H, —R³⁴, —CH₂—R³⁴ and —R³⁵.

Embodiment 160. The compound of any one of embodiments 1-67, 69 and 71-153, wherein R¹⁸ is selected from —CH₂—NR²²R²³, —R³⁴, —CH₂—R³⁴ and —R³⁵.

Embodiment 161. The compound of any one of embodiments 1-67, 69 and 71-153, wherein R¹⁸ is selected from —R³⁴, —CH₂—R³⁴ and —R³⁵.

Embodiment 162. The compound of any one of embodiments 1-67, 69 and 71-161, wherein R³⁴ is a 4-7 membered monocyclic heterocycle containing a nitrogen atom and zero, one or two additional heteroatoms selected from oxygen and sulfur, including sulfur dioxide, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 163. The compound of any one of embodiments 1-67, 69 and 71-162, wherein the monocyclic heterocycle of R³⁴ is substituted with 0 or 1 instance of methyl.

Embodiment 164. The compound of any one of embodiments 1-67, 69 and 71-162, wherein R³⁴ is selected from azetidinyl, pyrrolidinyl and morpholinyl substituted with 0 or 1 instance of methyl.

Embodiment 165. The compound of one of embodiments 1-67, 69 and 71-162, wherein R³⁴ is azetidinyl substituted with 0 or 1 instance of methyl.

Embodiment 166. The compound of any one of embodiments 1-67, 69 and 71-162, wherein R³⁴ is pyrrolidinyl substituted with 0 or 1 instance of methyl.

Embodiment 167. The compound of any one of embodiments 1-67, 69 and 71-162, wherein R³⁴ is morpholinyl substituted with 0 or 1 instance of methyl.

Embodiment 168. The compound of any one of embodiments 1-67, 69 and 71-162, wherein the attachment point for R³⁴ is a carbon atom.

Embodiment 169. The compound of embodiment 168, wherein R³⁴ is selected from the group consisting of:

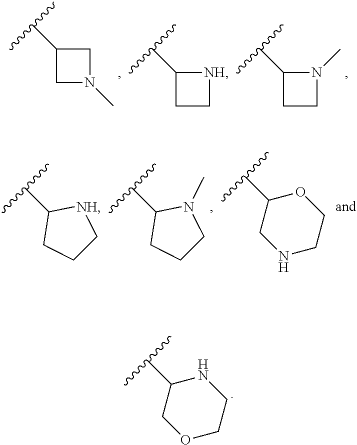

Embodiment 170. The compound of embodiment 168, wherein R³⁴ is selected from the group consisting of

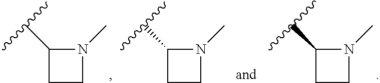

Embodiment 171. The compound of embodiment 168, wherein R³⁴ is

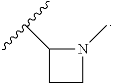 [SMILES: *C1CCN1C]

Embodiment 172. The compound of any one of embodiments 1-67, 69 and 71-161, wherein R³⁴ is a 4-10 membered heterocycle containing a nitrogen atom and zero, one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 173. The compound of embodiment 172, wherein R³⁴ is a 4-10 membered heterocycle containing a nitrogen atom and zero, one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 174. The compound of embodiment 172, wherein R³⁴ is a 4-8 member monocyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 175. The compound of embodiment 172, wherein R³⁴ is a 6-10 member fused bicyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 176. The compound of embodiment 172, wherein R³⁴ is a 6-10 member bridged heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 177. The compound of embodiment 172, wherein R³⁴ is a 6-10 member spiro heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 178. The compound of embodiment 172, wherein R³⁴ is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 2-oxa-5-azabicyclo[2.2.2]octane, 7-oxa-2-azabicyclo[3.3.1]nonane, 8-oxa-3-azabicyclo[3.2.1]octane, 9-oxa-3-azabicyclo[3.3.1]nonane, 6-oxa-8-azabicyclo[3.2.2]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 1,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 179. The compound of embodiment 178, wherein R³⁴ is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 180. The compound of any one of embodiments 172 to 179, wherein the attachment point for R³⁴ is the nitrogen atom of the heterocycle.

Embodiment 181. The compound of embodiment 180, wherein R³⁴ is selected from the group consisting of:

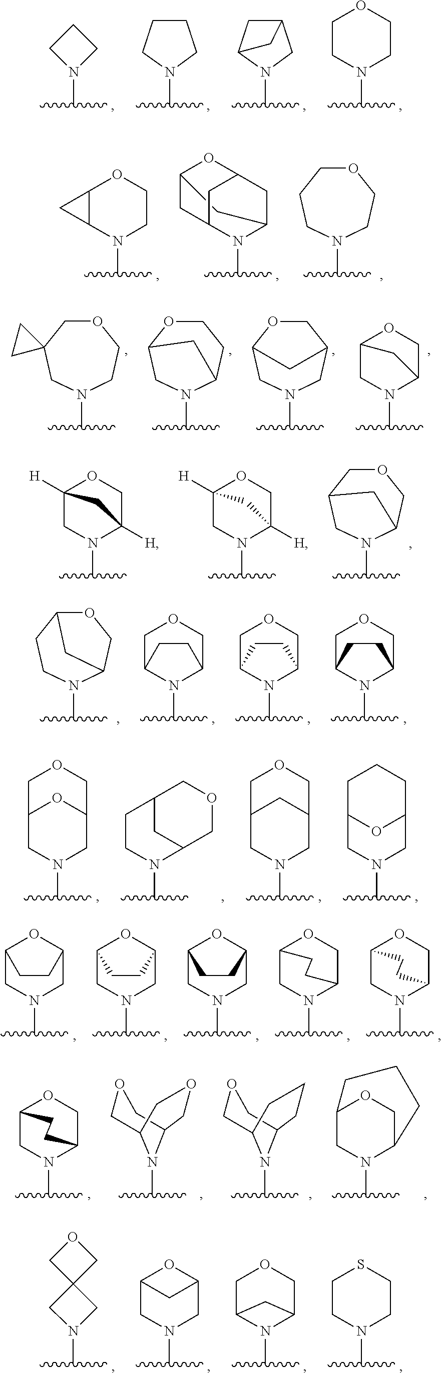

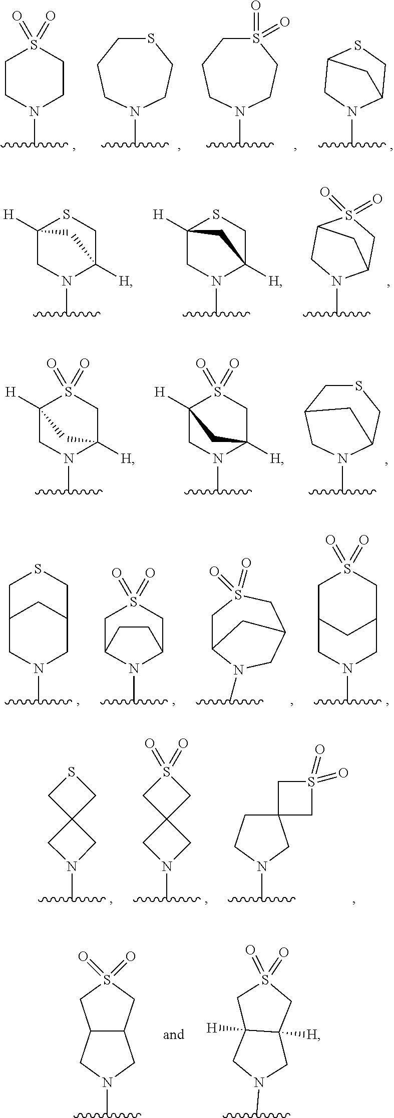

each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 182. The compound of embodiment 180, wherein R³⁴ is selected from the group consisting of:

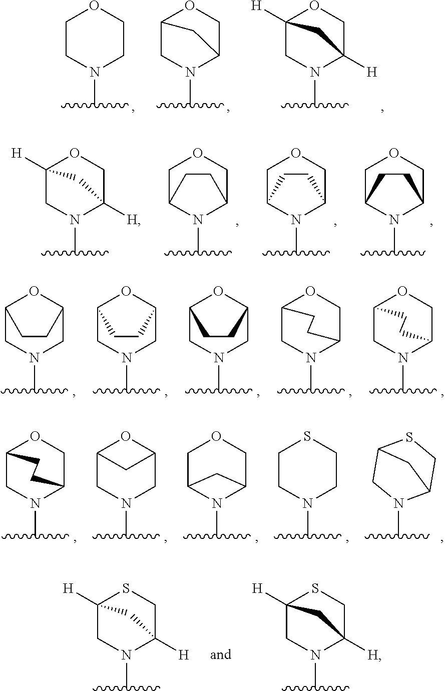

each
substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 183. The compound of embodiment 180, wherein R³⁴ is selected from the group consisting of:

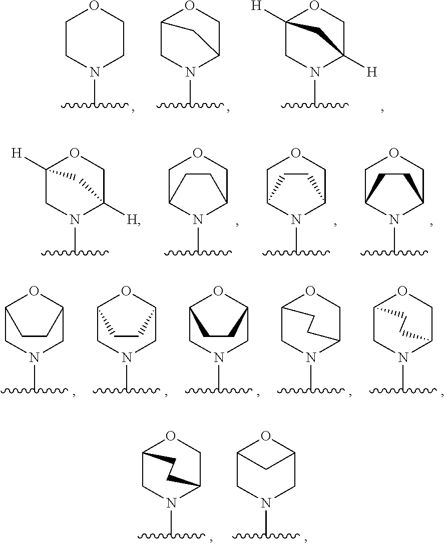

each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 184. The compound of embodiment 181, wherein R³⁴ is

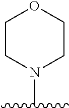 [SMILES: *N1CCOCC1]

substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 185. The compound of any one of embodiments 172 to 184, wherein the 4-10 membered heterocycle of R³⁴ is substituted with 0, 1, 2, 3 or 4 substituents independently selected from fluoro, methyl, ethyl, hydroxy, methoxy, trifluoromethyl, trifluoromethoxy, —CH₂OCH₃, —CH₂CH₂OCH₃, —CH₂CH₂OCH₂CH₂OCH₃, —CH₂N(CH₃)₂, and —CH₂CH₂N(CH₃)₂.

Embodiment 186. The compound of any one of embodiments 172 to 184, wherein the 4-10 membered heterocycle of R³⁴ is substituted with 0, 1 or 2 substituents independently selected from fluoro, methyl, ethyl, hydroxy, methoxy, trifluoromethyl, trifluoromethoxy, CH₂OCH₃, CH₂CH₂OCH₃, CH₂CH₂OCH₂CH₂OCH₃, CH₂N(CH₃)₂, and CH₂CH₂N(CH₃)₂.

Embodiment 187. The compound of any one of embodiments 172 to 184, wherein the 4-10 membered heterocycle of R³⁴ is substituted with 0, 1 or 2 substituents independently selected from fluoro and methyl.

Embodiment 188. The compound of any one of embodiments 172 to 184, wherein the 4-10 membered heterocycle of R³⁴ is unsubstituted.

Embodiment 189. The compound of any one of embodiments 172 to 184, wherein R³⁴ is selected from the group consisting of:

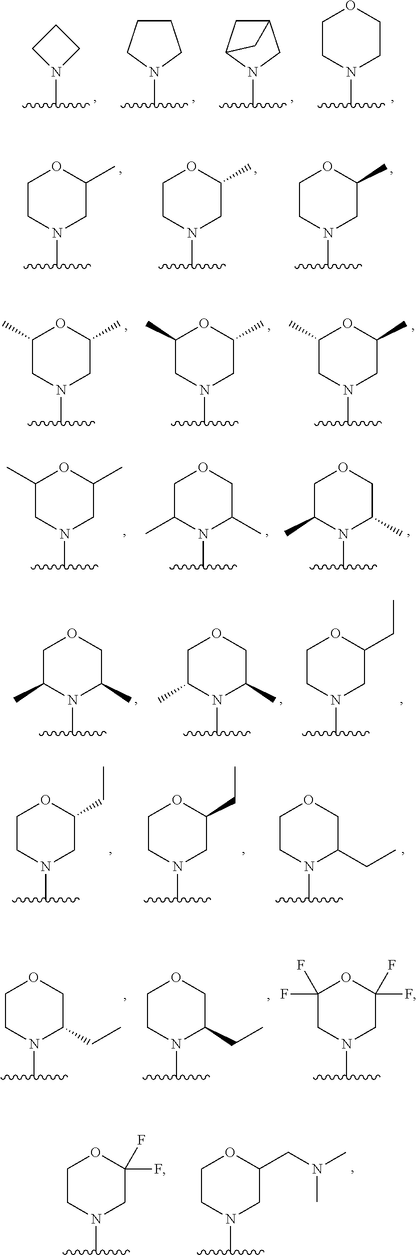

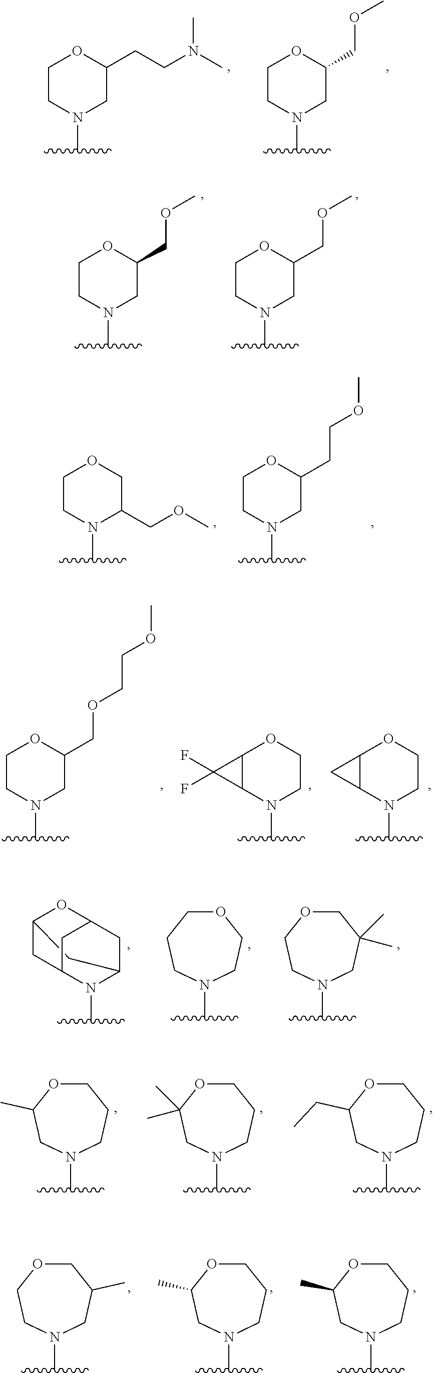

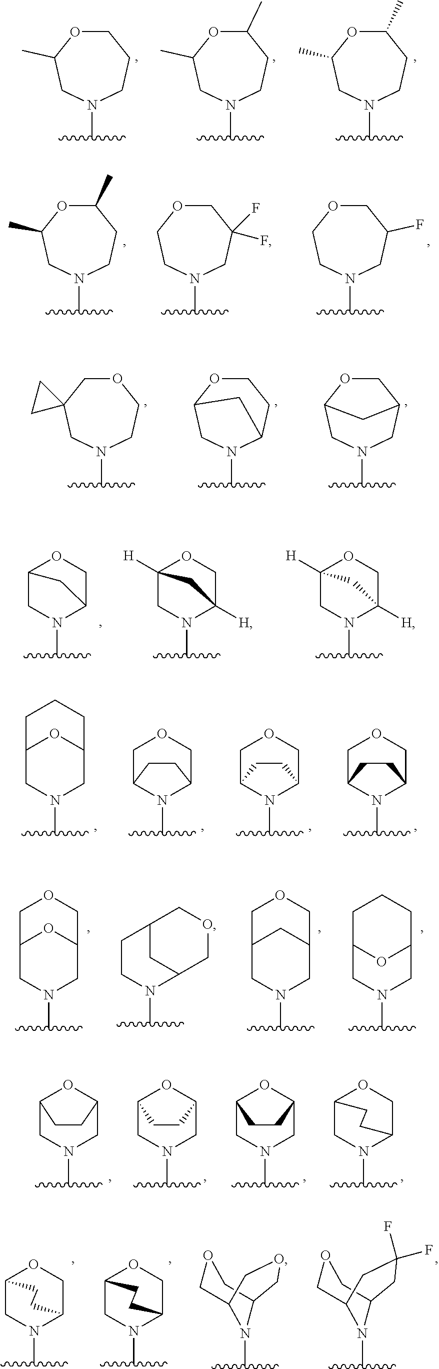

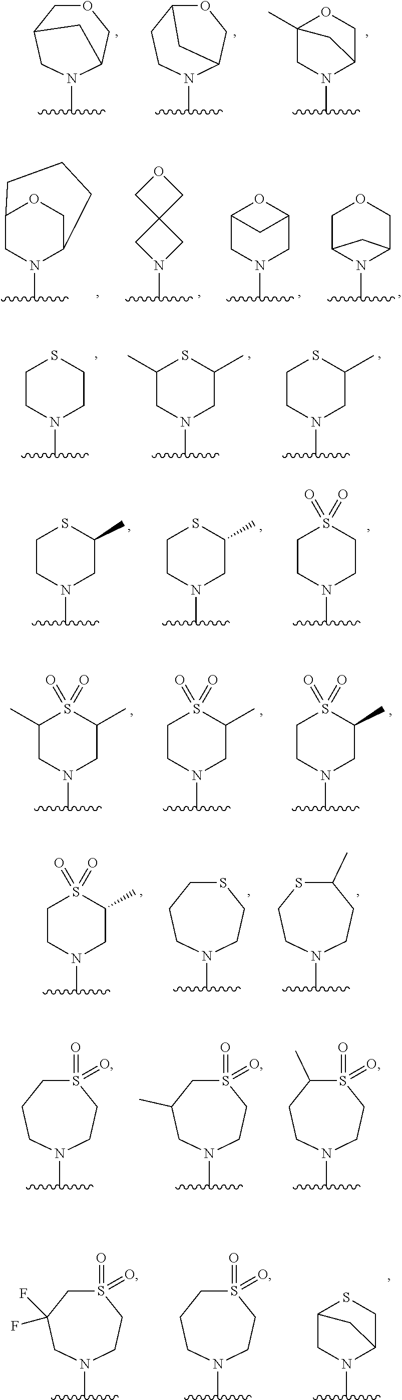

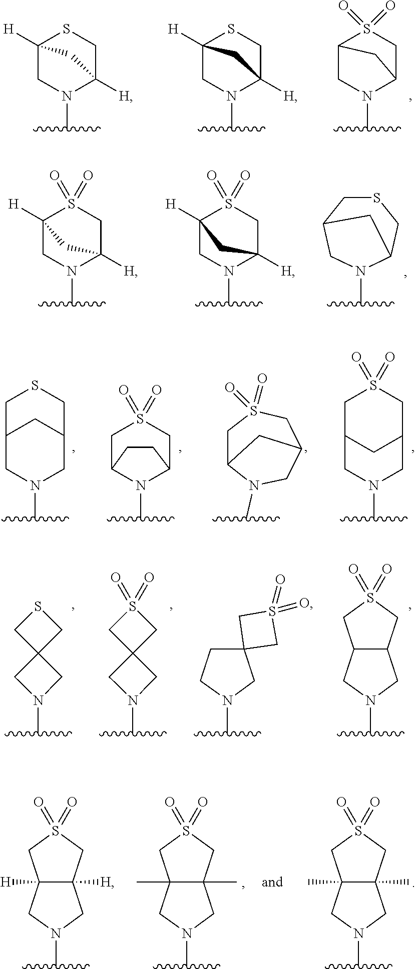

Embodiment 190. The compound of any one of embodiments 172 to 184, wherein R³⁴ is unsubstituted

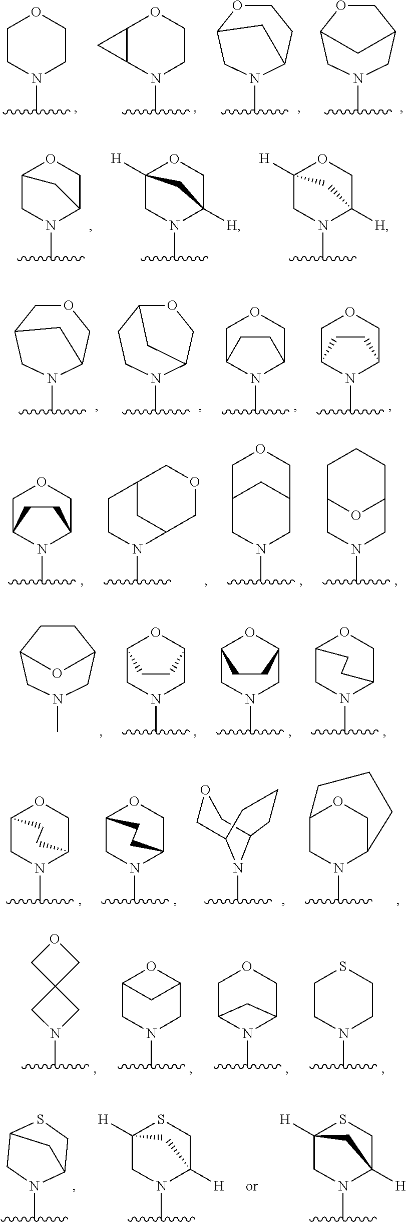

Embodiment 191. The compound of any one of embodiments 172 to 184, wherein R³⁴ is unsubstituted

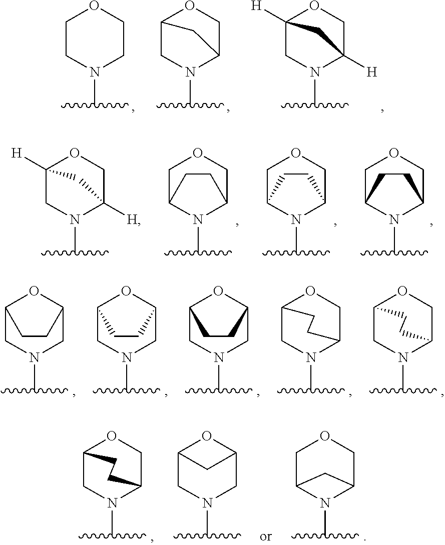

Embodiment 192. The compound of any one of embodiments 172 to 184, wherein R³⁴ is unsubstituted

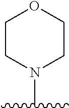 [SMILES: *N1CCOCC1]

Embodiment 193. The compound of any one of embodiments 172 to 192, wherein u is 1.

Embodiment 194. The compound of any one of embodiments 1-67, 69 and 71-147, 149 and 151-193, wherein R³⁵ is a 5-6 membered heteroaryl group containing at least one nitrogen atom, wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 195. The compound of any one of embodiments 1-67, 69 and 71-147, 149 and 151-193, wherein R³⁵ is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 196. The compound of any one of embodiments 1-67, 69 and 71-147, 149 and 151-193, wherein R³⁵ is selected from the group consisting of pyrimidinyl, oxazolyl, 1,2,4-oxadiazolyl, imidazolyl and 1,2,4-thiadiazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 197. The compound of any one of embodiments 1-67, 69 and 71-147, 149 and 151-193, wherein R³⁵ is selected from the group consisting of

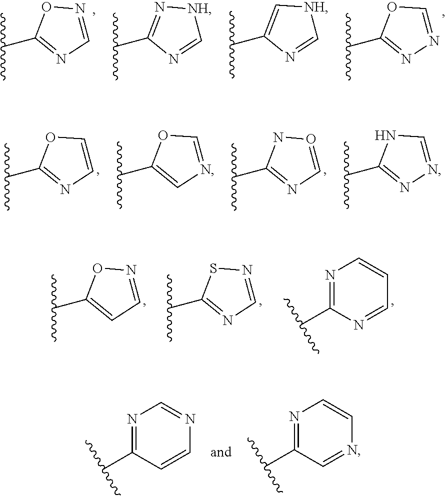

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 198. The compound of any one of embodiments 1-67, 69 and 71-147, 149 and 151-193, wherein R³⁵ is selected from the group consisting of

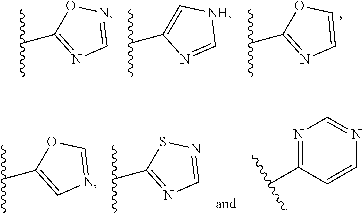

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 199. The compound of any one of embodiments 1-67, 69 and 71-147, 149 and 151-193, wherein R³⁵ is a 6 membered heteroaryl group substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 200. The compound of any one of embodiments 1-67, 69 and 71-147, 149 and 151-193, wherein R³⁵ is pyrimidinyl or pyridazinyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 201. The compound of any one of embodiments 1-67, 69 and 71-147, 149 and 151-193 wherein R³⁵ is selected from the group consisting of

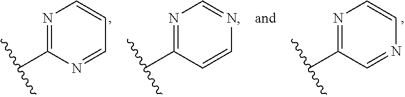

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 202. The compound of any one of embodiments 1-67, 69 and 71-147, 149 and 151-193, wherein R³⁵ is a 5 membered heteroaryl group containing at least one nitrogen atom, wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 203. The compound of any one of embodiments 1-67, 69 and 71-147, 149 and 151-193, wherein R³⁵ is selected from the group consisting of oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 204. The compound of any one of embodiments 1-67, 69 and 71-147, 149 and 151-193, wherein R³⁵ is selected from the group consisting of

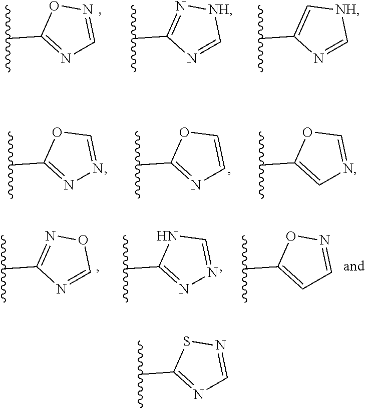

each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 205. The compound of any one of embodiments 1-67, 69 and 71-147, 149 and 151-193, wherein R³⁵ is selected from the group consisting of

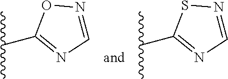 [SMILES: *c1ncno1 ; "and" ; *c1ncns1]

each substituted with 0 or 1 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 206. The compound of any one of embodiments 1-67, 69 and 71-147, 149 and 151-205, wherein the heteroaryl group of R³⁵ is substituted with 0 or 1 substituents selected from C₁-C₄ alkyl, C₁-C₄ hydroxyalkyl, C₁-C₄ haloalkyl, C₃-C₆ heterocyclyl optionally substituted with one or two substituents independently selected from halo and methyl, and C₃-C₆ cycloalkyl optionally substituted with one or two substituents independently selected from halo and methyl.

Embodiment 207. The compound of any one of embodiments 1-67, 69 and 71-147, 149 and 151-205, wherein the heteroaryl group of R³⁵ is substituted with 0 or 1 substituents selected from methyl, ethyl, isopropyl, tert-butyl, difluoromethyl, 1-fluoroethyl, 1,1-difluoroethyl, —C(OH)(CH₃)₂, oxetan-3-yl, 3-methyloxetan-3-yl, cyclobutyl, cyclopropyl, 1-methylcyclopropyl and 2-fluorocyclopropyl.

Embodiment 208. The compound of any one of embodiments 1-67, 69 and 71-147, 149 and 151-193, wherein R³⁵ is selected from the group consisting of:

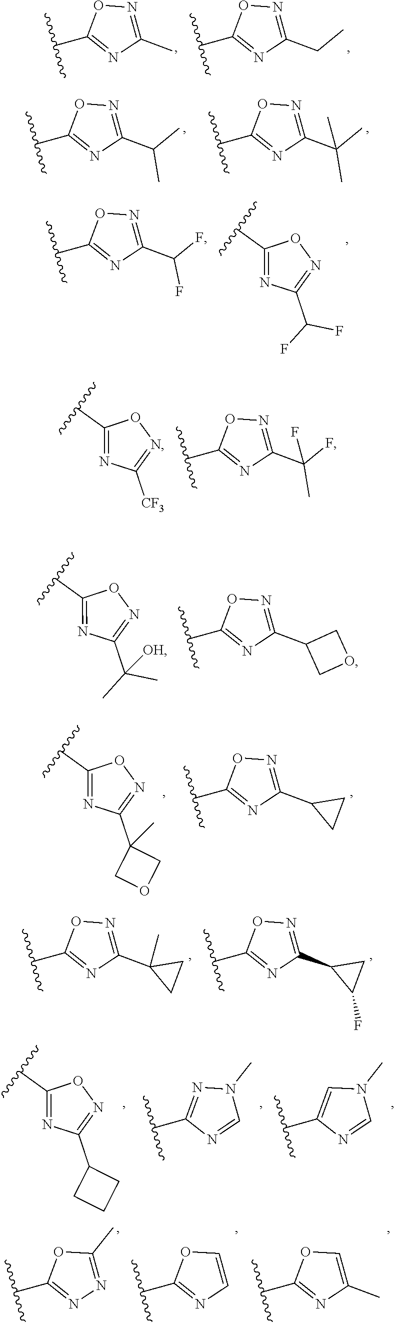

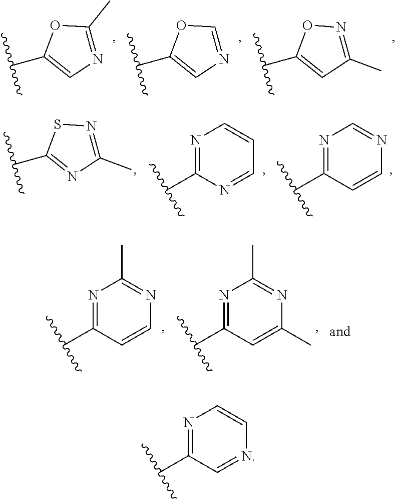

Embodiment 209. The compound of any one of embodiments 1-67, 69 and 71-147, 149 and 151-193, wherein R³⁵ is selected from the group consisting of:

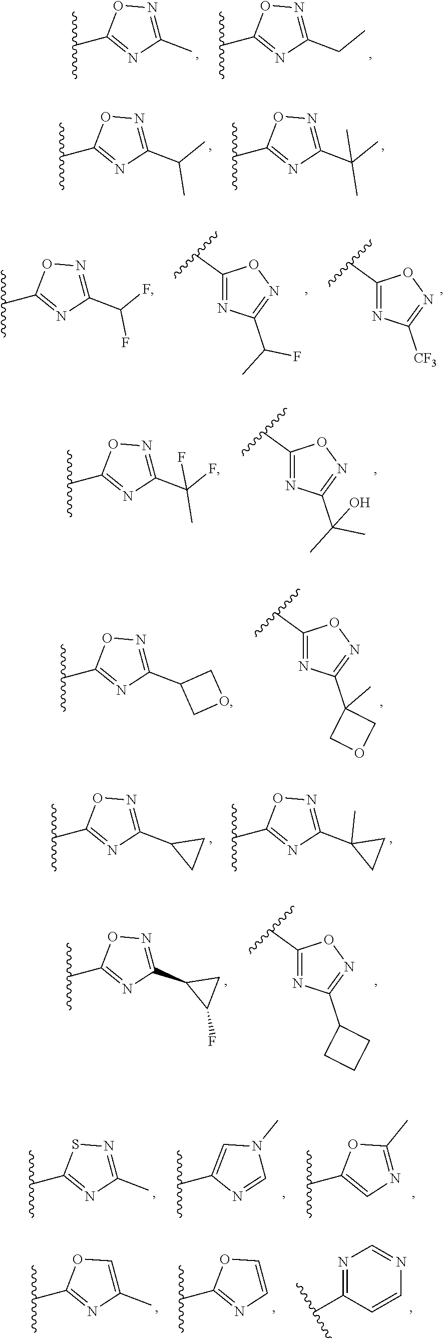

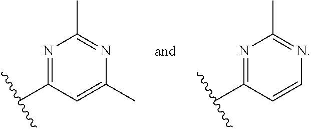

Embodiment 210. The compound of any one of embodiments 1-67, 69 and 71-147, 149 and 151-193, wherein R³⁵ is selected from the group consisting of:

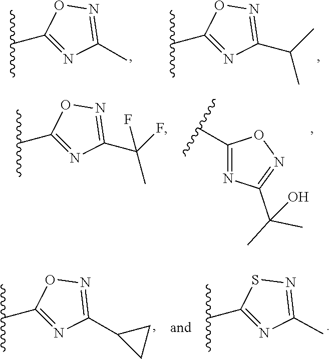

Embodiment 211. The compound of any one of embodiments 1-67, 69 and 71-147, 149 and 151-193, wherein R³⁵ is 1,2,4-oxadiazolyl substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl and C₃-C₆ cycloalkyl optionally substituted with one or two substituents independently selected from halo and methyl.

Embodiment 212. The compound of any one of embodiments 1-67, 69 and 71-147, 149 and 151-193, wherein R³⁵ is

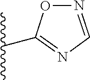 [SMILES: *c1ncno1]

substituted with 1 substituent selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

Embodiment 213. The compound of embodiments 211 or 212, wherein the oxadiazolyl is substituted with one substituent selected from methyl, ethyl, isopropyl, tert-butyl, difluoromethyl, 1-fluoroethyl, 1,1-difluoroethyl, —C(OH)(CH₃)₂, oxetan-3-yl, 3-methyloxetan-3-yl, cyclobutyl, cyclopropyl, 1-methylcyclopropyl and 2-fluorocyclopropyl.

Embodiment 214. The compound of any one of embodiments 1-67, 69 and 71-147, 149 and 151-193, wherein R³⁵ is selected from the group consisting of:

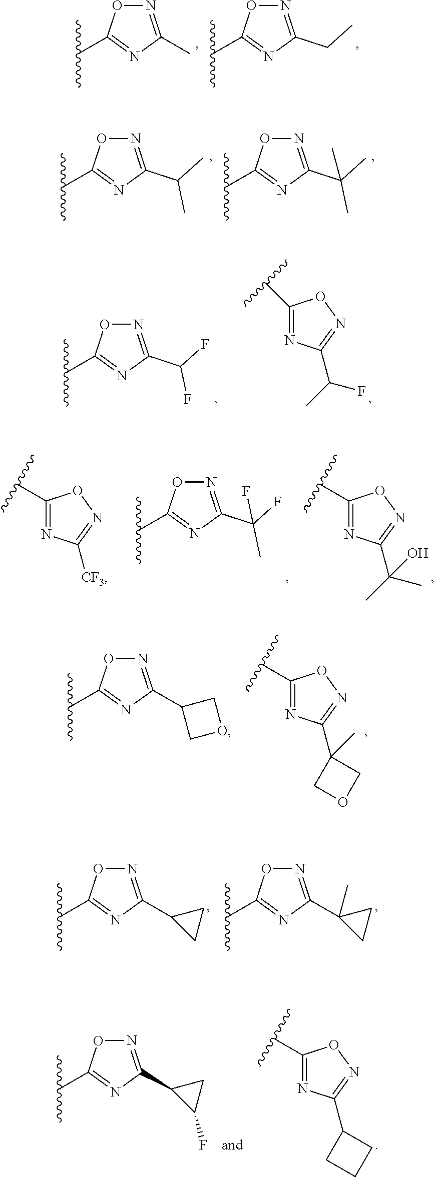

Embodiment 215. The compound of any one of embodiments 1-67, 69 and 71-147, 149 and 151-193, wherein R³⁵ is selected from the group consisting of:

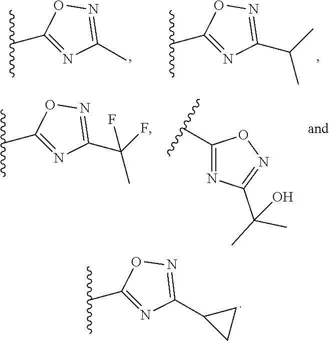

Embodiment 216. The compound of any one of embodiments 1-67, 69 and 71-147, 149 and 151-193 wherein R³⁵ is pyrimidinyl or pyridazinyl substituted with 0, 1 or 2 instances of methyl.

Embodiment 217. The compound of any one of embodiments 1-67, 69 and 71-147, 149 and 151-193, wherein R³⁵ is selected from:

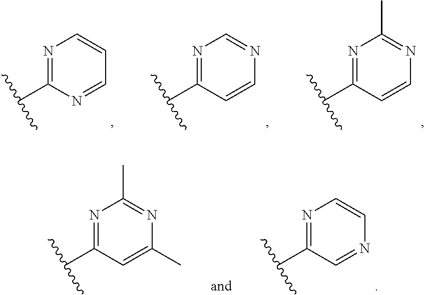

Embodiment 218. The compound of any one of embodiments 1-67, 69 and 71-147, 149 and 151-193, wherein R³⁵ is selected from:

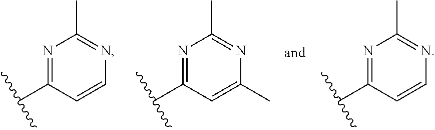

Embodiment 219. The compound of any one of embodiments 1-67, 69 and 71-147, 149 and 151-193, wherein R³⁵ is selected from:

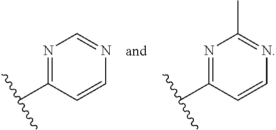

Embodiment 220. The compound of any one of embodiments 1-67, 69 and 71-147, 149 and 151-219, wherein the attachment point for R³⁵ is on a carbon atom.

Embodiment 221. The compound of any one of embodiments 1-67, 69 and 71-146 and 162-192, wherein R¹⁸ is —(CH₂)ᵤR³⁴.

Embodiment 222. The compound of any one of embodiments 1-67, 69 and 71-146 and 162-192, wherein R¹⁸ is —CH₂—R³⁴.

Embodiment 223. The compound of any one of embodiments 1-67, 69 and 71-146 and 162-192, wherein R¹⁸ is R³⁴.

Embodiment 224. The compound of any one of embodiments 1-67, 69 and 71-146 and 194-220, wherein R¹⁸ is R.

Embodiment 225. The compound of any one of embodiments 1-67, 69 and 71-146, wherein R¹⁸ is —CH₂N(CH₃).

Embodiment 226. The compound of any one of embodiments 1-67, 69 and 71-146, wherein R¹⁸ is H.

Embodiment 227. The compound of any one of embodiments 1-67, 69 and 71-220, wherein R¹⁸ is not H.

Embodiment 228. The compound of any one of embodiments 1-67, 69 and 71-146, wherein R¹⁸ is selected from the group consisting of hydrogen, —COOH, —C(O)OCH₃, —C(O)OCH₂CH₃, —C(O)OCH(CH₃)₂, —C(O)N(CH₃)₂, —C(O)-cyclopropyl, —CH₂OCH₃, —CH₂N(CH₃)₂, —S(O)₂CH₃, —S(O)₂CH₂CH₃, —S(O)₂-cyclopropyl,

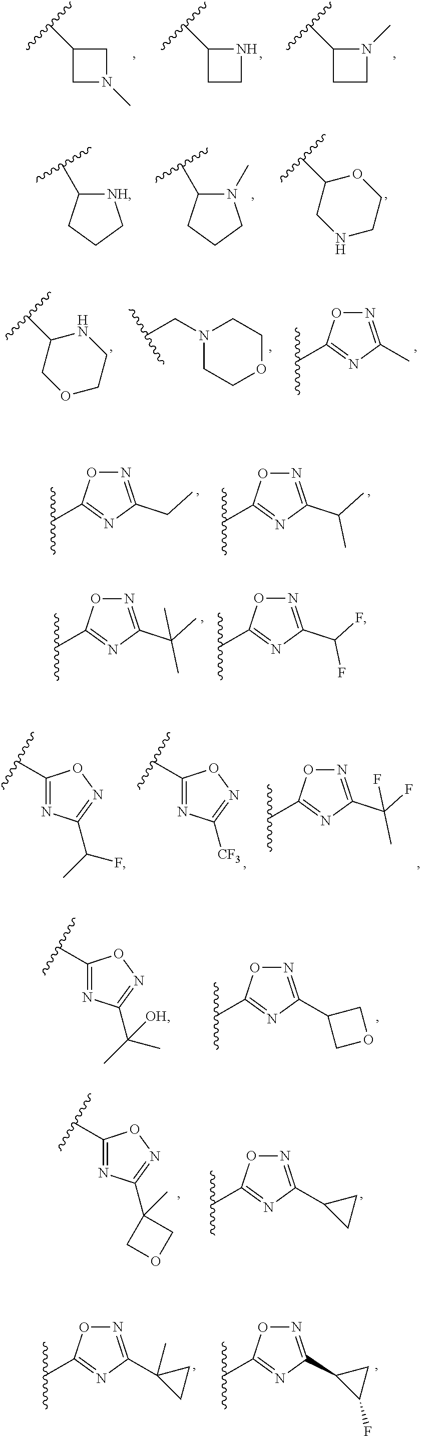

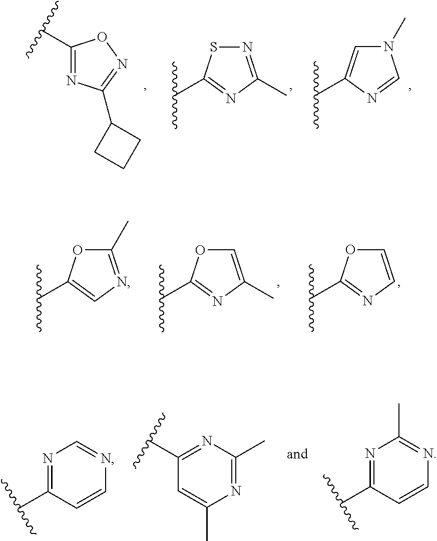

Embodiment 229. The compound of any one of embodiments 1-67, 69 and 71-146, wherein R¹⁸ is selected from the group consisting of hydrogen, —COOH, —C(O)OCH₃, —C(O)OCH₂CH₃, —C(O)OCH(CH₃)₂, —C(O)N(CH₃)₂, —C(O)-cyclopropyl, —CH₂OCH₃, —CH₂N(CH₃)₂, —S(O)₂CH₃, —S(O)₂CH₂CH₃, —S(O)₂-cyclopropyl,

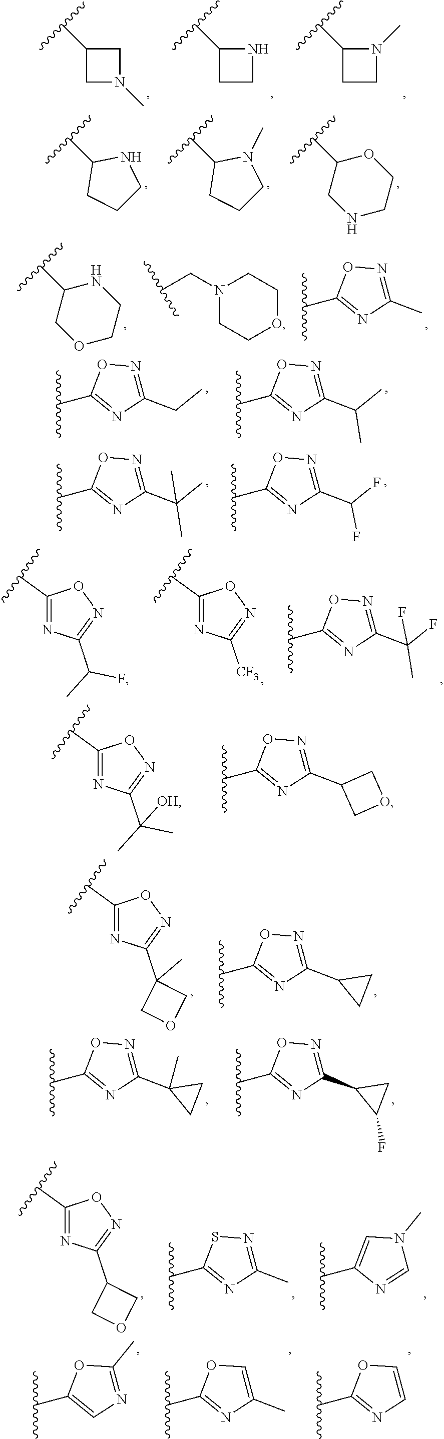

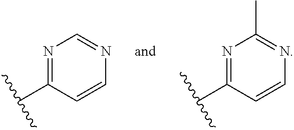

Embodiment 230. The compound of any one of embodiments 1-67, 69 and 71-146, wherein R¹⁸ is selected from the group consisting of hydrogen, —CH₂N(CH₃)₂,

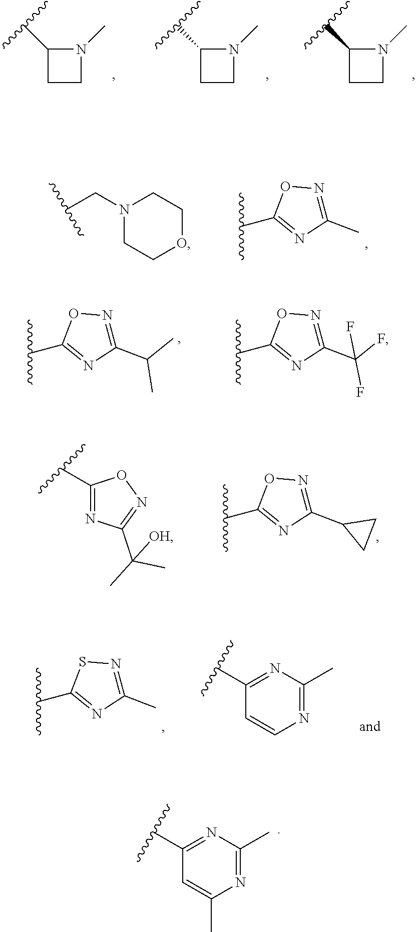

Embodiment 231. The compound of any one of embodiments 1-67, 69 and 71-146, wherein R¹⁸ is —CH₂N(CH₃)₂.

Embodiment 232. The compound of any one of embodiments 1-67, 69 and 71-146, wherein R¹⁸ is

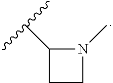 [SMILES: *C1CCN1C]

Embodiment 233. The compound of any one of embodiments 1-67, 69 and 71-146, wherein R¹⁸ is

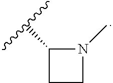 [SMILES: *[C@H]1CCN1C]

Embodiment 234. The compound of any one of embodiments 1-67, 69 and 71-146, wherein R¹⁸ is

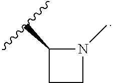 [SMILES: *[C@@H]1CCN1C]

Embodiment 235. The compound of any one of embodiments 1-67, 69 and 71-146, wherein R¹⁸ is

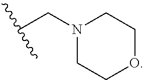 [SMILES: *CN1CCOCC1]

Embodiment 236. The compound of any one of embodiments 1-67, 69 and 71-146, wherein R¹⁸ is

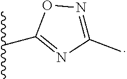 [SMILES: *c1nc(C)no1]

Embodiment 237. The compound of any one of embodiments 1-67, 69 and 71-146, wherein R¹⁸ is

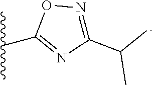 [SMILES: *c1nc(C(C)C)no1]

Embodiment 238. The compound of any one of embodiments 1-67, 69 and 71-146, wherein R¹⁸ is

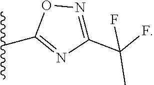 [SMILES: *c1nc(C(C)(F)F)no1]

Embodiment 239. The compound of any one of embodiments 1-67, 69 and 71-146, wherein R¹⁸ is

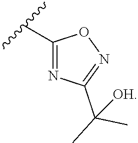 [SMILES: *c1nc(C(C)(C)O)no1]

Embodiment 240. The compound of any one of embodiments 1-67, 69 and 71-146, wherein R¹⁸ is

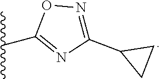 [SMILES: *c1nc(C2CC2)no1]

Embodiment 241. The compound of any one of embodiments 1-67, 69 and 71-146, wherein R¹⁸ is

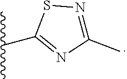 [SMILES: *c1nc(C)ns1]

Embodiment 242. The compound of any one of embodiments 1-67, 69 and 71-146, wherein R¹⁸ is

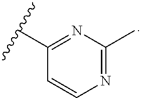 [SMILES: *c1ccnc(C)n1]

Embodiment 243. The compound of any one of embodiments 1-67, 69 and 71-146, wherein R¹⁸ is

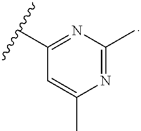 [SMILES: *c1cc(C)nc(C)n1]

Embodiment 244. The compound of any one of embodiments 1-67, 69 and 71-146, wherein R¹⁸ is selected from the group consisting of hydrogen, —CH₂N(CH₃)₂,

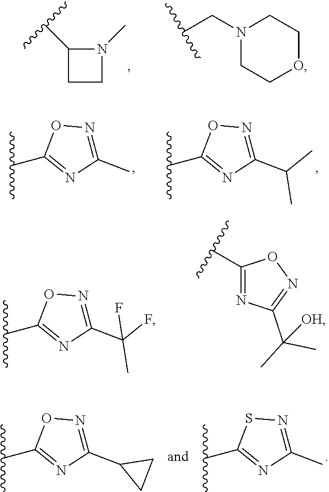

Embodiment 245. The compound of any one of embodiments 1-67, 69 and 71-244, wherein the double bond in the —C(O)C(R¹⁹)═C(R²⁰)R¹⁸ portion of the compound is in the E configuration.

Embodiment 246. The compound of any one of embodiments 1-66, wherein R² is R^{2e}.

Embodiment 247. The compound of any one of embodiments 1-66, 68, 69, 71-84 and 86-246, wherein R²⁸ is selected from the group consisting of C₁-C₄ alkyl and C₁-C₄ alkoxy.

Embodiment 248. The compound of any one of embodiments 1-66, 68, 69, 71-84 and 86-246, wherein R²⁸ is selected from the group consisting of methyl, ethyl, and —CH₂CH₂OCH₃.

Embodiment 249. The compound of any one of embodiments 1-66, 68, 69, 71-84 and 86-246, wherein R²⁸ is methyl or ethyl.

Embodiment 250. The compound of any one of embodiments 1-66, 68, 69, 71-84 and 86-246, wherein R²⁸ is methyl.

Embodiment 251. The compound of any one of embodiments 1-66, 68, 69, 71-84 and 86-250, wherein t is 0 or 1.

Embodiment 252. The compound of any one of embodiments 1-66, 68, 69, 71-84 and 86-250, wherein t is 1.

Embodiment 253. The compound of any one of embodiments 1-66, 68, 69, 71-84 and 86-250, wherein t is 0.

Embodiment 254. The compound of any one of embodiments 1-66, 68, 69, 71-84 and 86-253, wherein R³⁰ is a 4-5 membered monocyclic saturated heterocyclic group comprising one nitrogen as the sole heteroatom within the ring atoms, wherein the nitrogen ring atom of the heterocyclic group is substituted with —C(O)C≡CR³¹ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 255. The compound of any one of embodiments 1-66, 68, 69, 71-84 and 86-253 wherein the heterocyclic group of R³⁰ is not further substituted.

Embodiment 256. The compound of any one of embodiments 1-66, 68, 69, 71-84 and 86-254 wherein the heterocyclic group of R³⁰ is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 257. The compound of any one of embodiments 1-66, 68, 69, 71-84 and 86-253, wherein the heterocyclic group of R³⁰ is selected from the group consisting of:

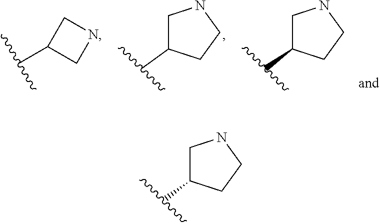

wherein the ring nitrogen of the heterocyclic group is substituted with —C(O)C≡CR³¹ and the heterocyclic group is not further substituted, or is substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 258. The compound of embodiment 257, wherein the heterocyclic group of R³⁰ is not further substituted.

Embodiment 259. The compound of embodiment 257, wherein the heterocyclic group of R³⁰ is further substituted with 1 substituent selected from the group consisting of hydroxy, CN, C₁-C₄ alkyl, C₁-C₄ cyanoalkyl, and halo.

Embodiment 260. The compound of any one of embodiments 1-66, 68, 69, 71-84 and 86-250, wherein R²⁹ is selected from the group consisting of:

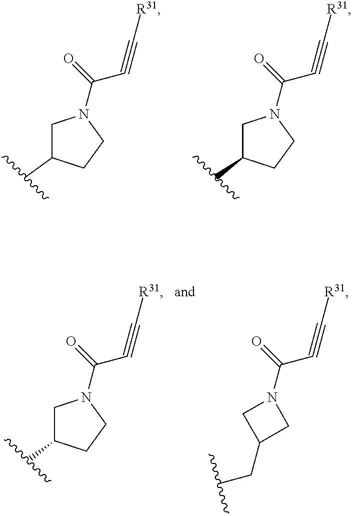

wherein the azetidine and pyrrolidine groups are not further substituted, or are substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

Embodiment 261. The compound of embodiment 260, wherein the azetidine and pyrrolidine groups are not further substituted.

Embodiment 262. The compound of embodiment 260, wherein the azetidine and pyrrolidine groups are further substituted with 1 substituent selected from the group consisting of hydroxy, CN, C₁-C₄ alkyl, C₁-C₄ cyanoalkyl, and halo.

Embodiment 263. The compound of any one of embodiments 1-66, 68, 69, 71-84 and 86-262 wherein v is 1.

Embodiment 264. The compound of any one of embodiments 1-66, 68, 69, 71-84 and 86-262 wherein v is 2.

Embodiment 265. The compound of any one of embodiments 1-66, 68, 69, 71-84 and 86-264 wherein p is 0 or 1.

Embodiment 266. The compound of any one of embodiments 1-66, 68, 69, 71-84 and 86-264 wherein p is 0.

Embodiment 267. The compound of any one of embodiments 1-66, 68, 69, 71-84 and 86-264 wherein p is 1.

Embodiment 268. The compound of any one of embodiments 1-66, 68, 69, 71-84 and 86-262 and 265-267, wherein R³¹ is selected from the group consisting of —CH₂—NR³²R³³ and —(CH₂)ₚ—R³⁶

Embodiment 269. The compound of any one of embodiments 1-66, 68, 69, 71-84 and 86-262, wherein R³¹ is selected from the group consisting of —CH₂—NR³²R³³ and —CH₂—R³⁶.

Embodiment 270. The compound of any one of embodiments 1-66, 68, 69, 71-84 and 86-269, wherein R³² and R³³ are independently selected from methyl and ethyl.

Embodiment 271. The compound of any one of embodiments 11-66, 68, 69, 71-84 and 86-262 and 265-267 wherein R³¹ is —(CH₂)ₚ—R³⁶.

Embodiment 272. The compound of any one of embodiments 1-66, 68, 69, 71-84 and 86-264, wherein R³¹ is —(CH₂)ᵥ—NR³²R³³.

Embodiment 273. The compound of any one of embodiments 1-66, 68, 69, 71-84 and 86-264, wherein R³¹ is —CH₂—NR³²R³³.

Embodiment 274. The compound of any one of embodiments 1-66, 68, 69, 71-84 and 86-271, wherein R³⁶ is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 275. The compound of embodiment 274, wherein the monocyclic heterocycle of R³⁶ is substituted with 0 or 1 instance of methyl.

Embodiment 276. The compound of embodiment 275, wherein R³⁶ is selected from azetidinyl, pyrrolidinyl and morpholinyl substituted with 0 or 1 instance of methyl.

Embodiment 277. The compound of embodiment 275, wherein R³⁶ is azetidinyl substituted with 0 or 1 instance of methyl.

Embodiment 278. The compound of embodiment 275, wherein R³⁶ is pyrrolidinyl substituted with 0 or 1 instance of methyl.

Embodiment 279. The compound of embodiment 275, wherein R³⁶ is morpholinyl substituted with 0 or 1 instance of methyl.

Embodiment 280. The compound of any one of embodiments 1-66, 68, 69, 71-84 and 86-271 and 274-279, wherein the attachment point for R³⁶ is a carbon atom.

Embodiment 281. The compound of embodiment 281, wherein R³⁶ is selected from the group consisting of:

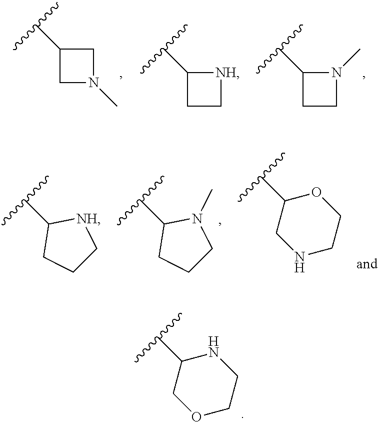

Embodiment 282. The compound of any one of embodiments 274-281, wherein p is 0.

Embodiment 283. The compound of any one of embodiments 1-66, 68, 69, 71-84 and 86-271, wherein R³⁶ is a 4-10 membered heterocycle containing a nitrogen atom and zero, one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 284. The compound of embodiment 283, wherein R³⁶ is a 4-10 membered heterocycle containing a nitrogen atom and one or two additional heteroatoms selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 285. The compound of embodiment 283, wherein R³⁶ is a 4-8 member monocyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 286. The compound of embodiment 283, wherein R³⁶ is a 6-10 member fused bicyclic heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 287. The compound of embodiment 283, wherein R³⁶ is a 6-10 member bridged heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 288. The compound of embodiment 283, wherein R³⁶ is a 6-10 member spiro heterocycle substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 289. The compound of any one of embodiments 283-288, wherein R³⁶ is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 2-oxa-5-azabicyclo[2.2.2]octane, 7-oxa-2-azabicyclo[3.3.1]nonane, 8-oxa-3-azabicyclo[3.2.1]octane, 9-oxa-3-azabicyclo[3.3.1]nonane, 6-oxa-8-azabicyclo[3.2.2]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 1,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 290. The compound of embodiment 289, wherein R³⁶ is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 291. The compound of any one of embodiments 283-290, wherein the attachment point for R³⁶ is the nitrogen atom of the heterocycle.

Embodiment 292. The compound of embodiment 291, wherein the R³⁶ is selected from the group consisting of:

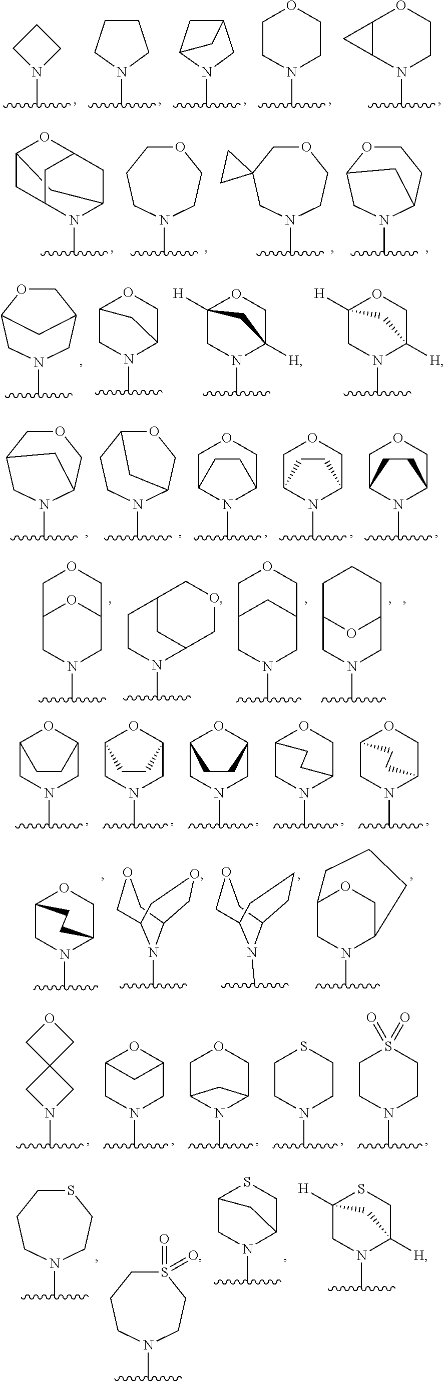

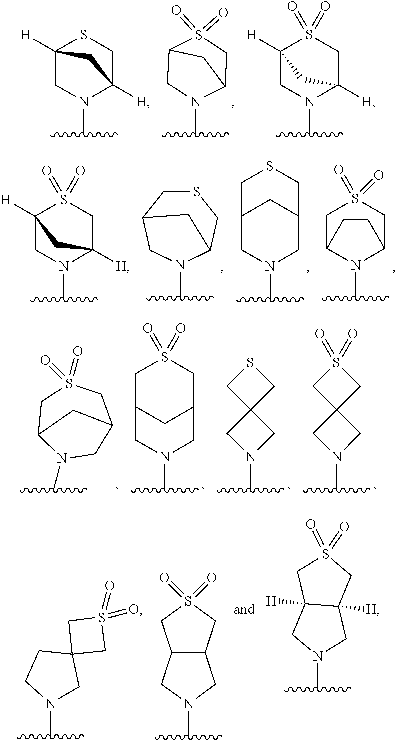

each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 293. The compound of embodiment 292, wherein R³⁶ is

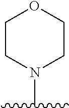 [SMILES: *N1CCOCC1]

substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

Embodiment 294. The compound of any one of embodiments 283-292, wherein the 4-10 membered heterocycle of R³⁶ is substituted with 0, 1, 2, 3 or 4 substituents independently selected from fluoro, methyl, ethyl, hydroxy, methoxy, trifluoromethyl, trifluoromethoxy, —CH₂OCH₃, —CH₂CH₂OCH₃, —CH₂CH₂OCH₂CH₂OCH₃, —CH₂N(CH₃)₂, and —CH₂CH₂N(CH₃)₂.

Embodiment 295. The compound of any one of embodiments 283-292, wherein the 4-10 membered heterocycle of R³⁶ is substituted with 0, 1 or 2 substituents independently selected from fluoro, methyl, ethyl, hydroxy, methoxy, trifluoromethyl, trifluoromethoxy, —CH₂OCH₃, —CH₂CH₂OCH₃, —CH₂CH₂OCH₂CH₂OCH₃, —CH₂N(CH₃)₂, and —CH₂CH₂N(CH₃)₂.

Embodiment 296. The compound of any one of embodiments 283-292, wherein the 4-10 membered heterocycle of R³⁶ is substituted with 0, 1 or 2 substituents independently selected from fluoro and methyl.

Embodiment 297. The compound of any one of embodiments 283-292, wherein the 4-10 membered heterocycle of R³⁶ is unsubstituted.

Embodiment 298. The compound of any one of embodiments 283-292, wherein R³⁶ is selected from the group consisting of.

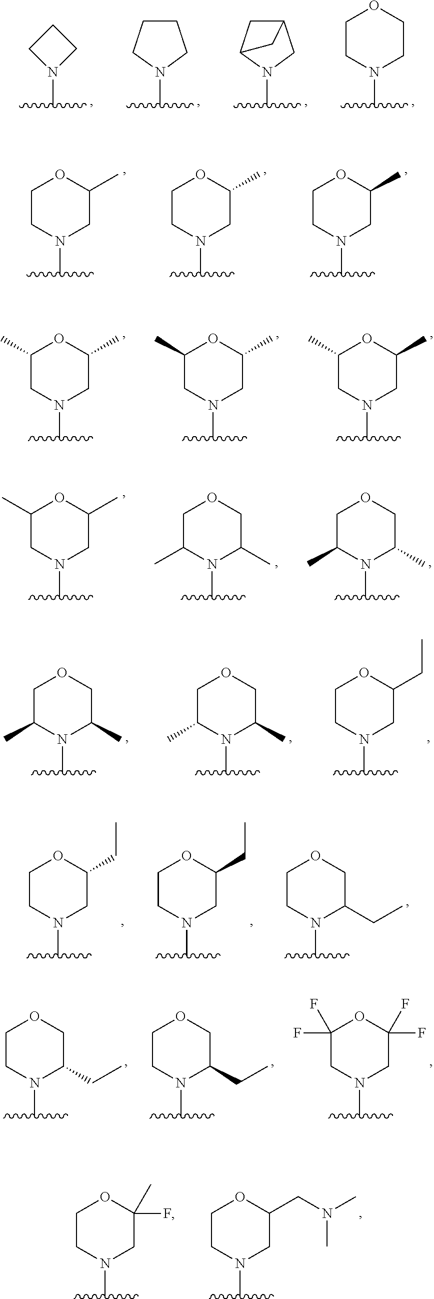

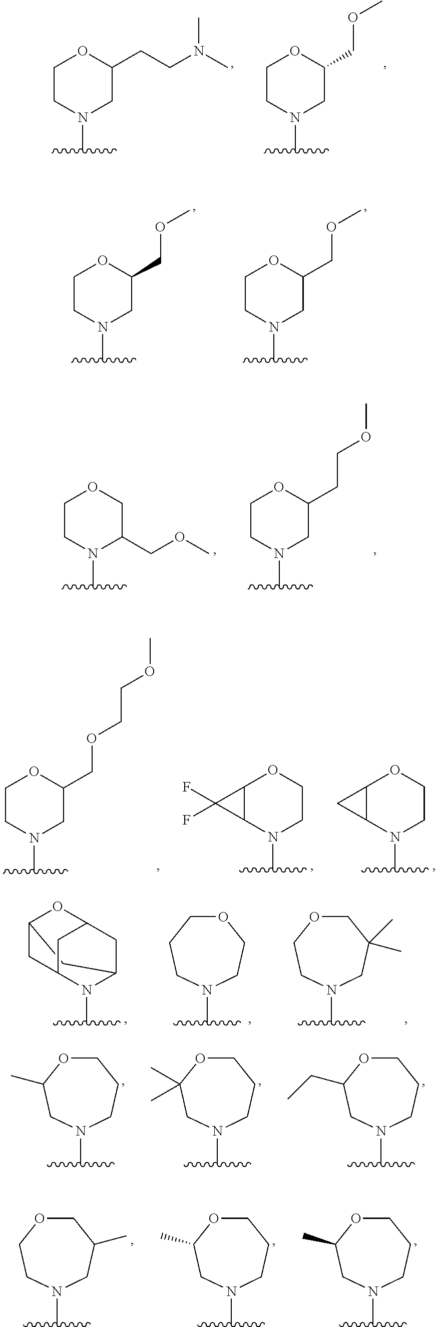

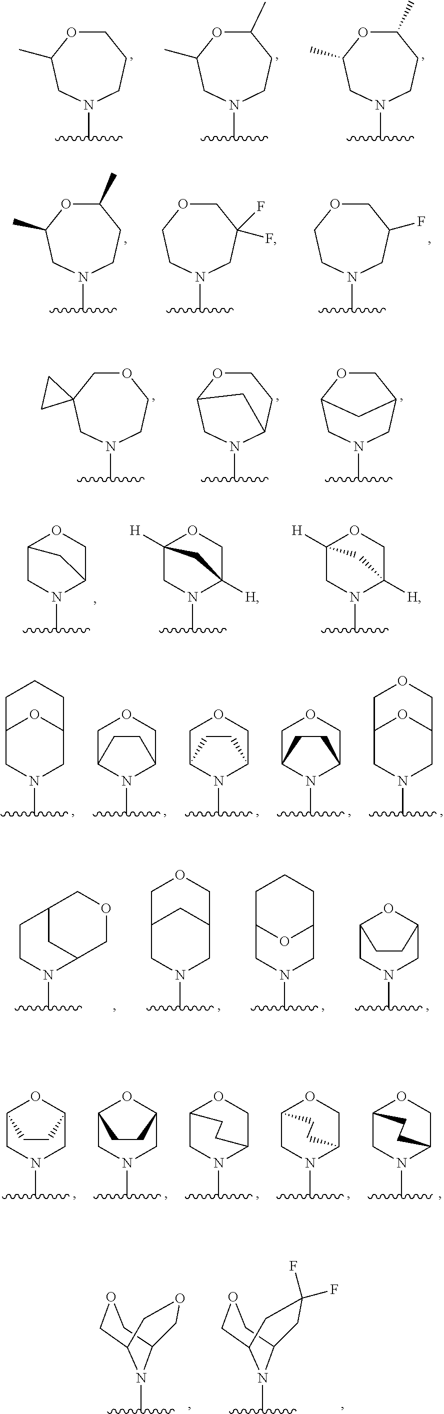

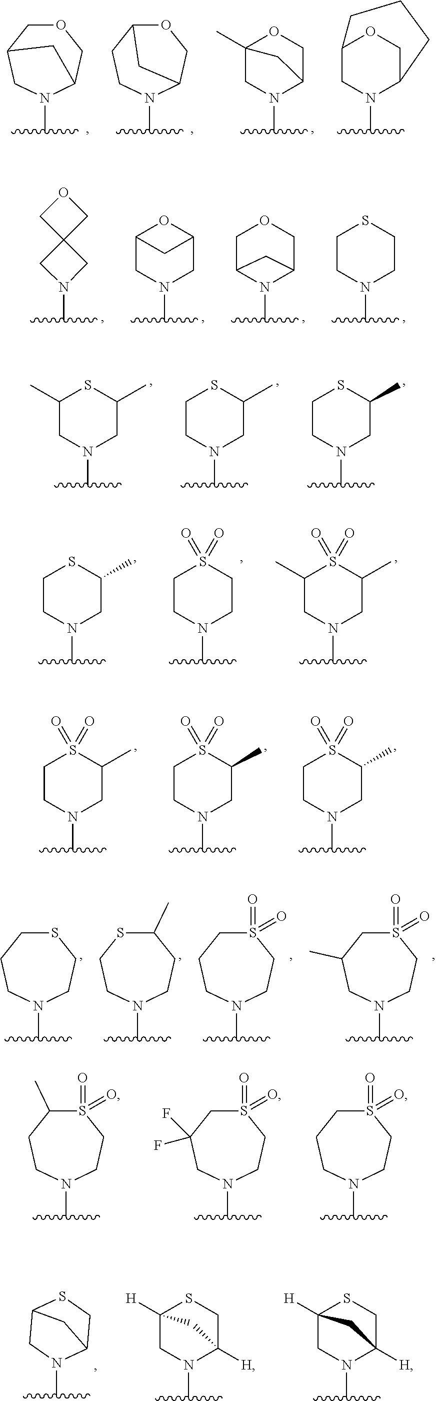

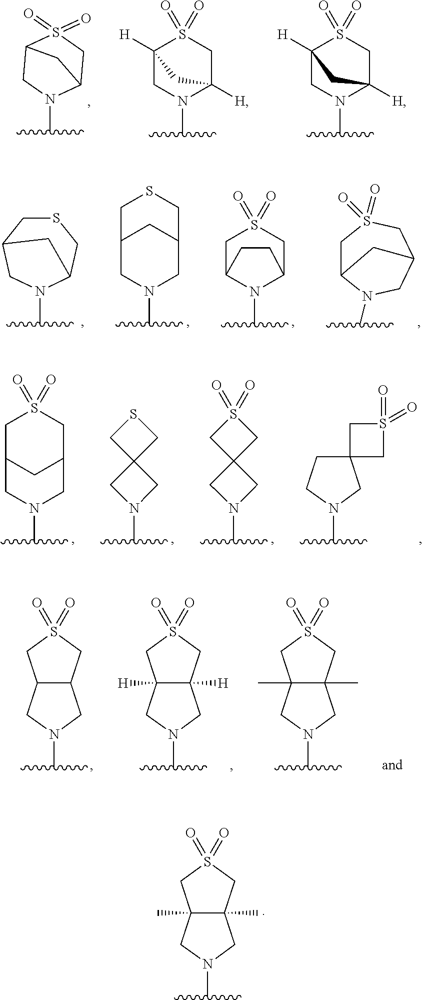

Embodiment 299. The compound of any one of embodiments 283-292, wherein R³⁶ is unsubstituted

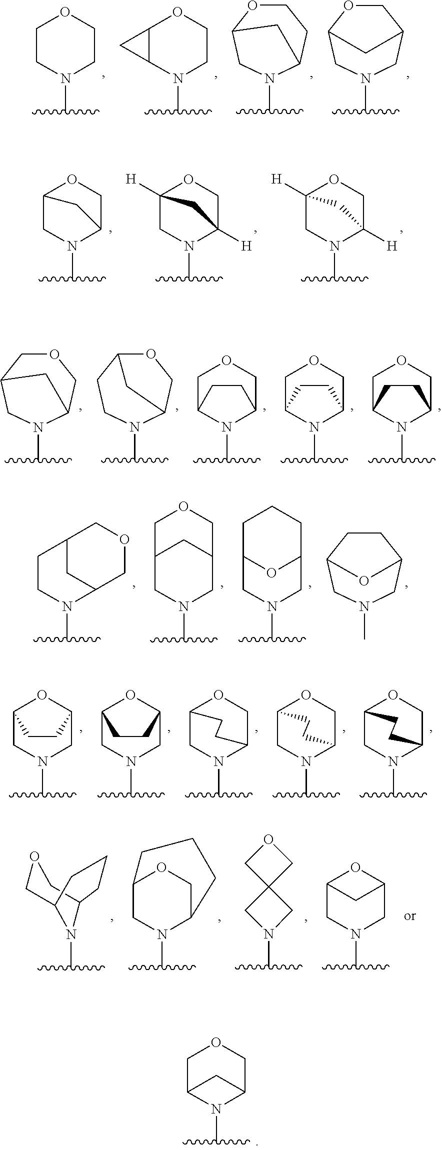

Embodiment 300. The compound of any one of embodiments 283-292, wherein R³⁶ is unsubstituted

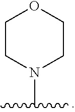 [SMILES: *N1CCOCC1]

Embodiment 301. The compound of any one of embodiments 285-300, wherein p is 1.

Embodiment 302. The compound of any one of embodiments 1-66, 68, 69, 71-84 and 86-262, wherein R³¹ is selected from the group consisting of:

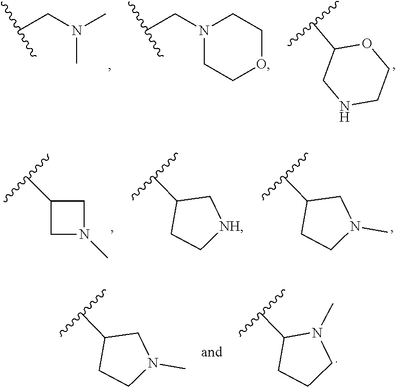

Embodiment 303. The compound of any one of embodiments 1-67, 69, 70, 72-85 and 87-263, wherein R³¹ is selected from the group consisting of:

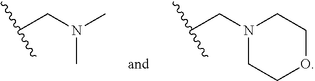

Embodiment 304. The compound of embodiment 1, selected from the group consisting of:

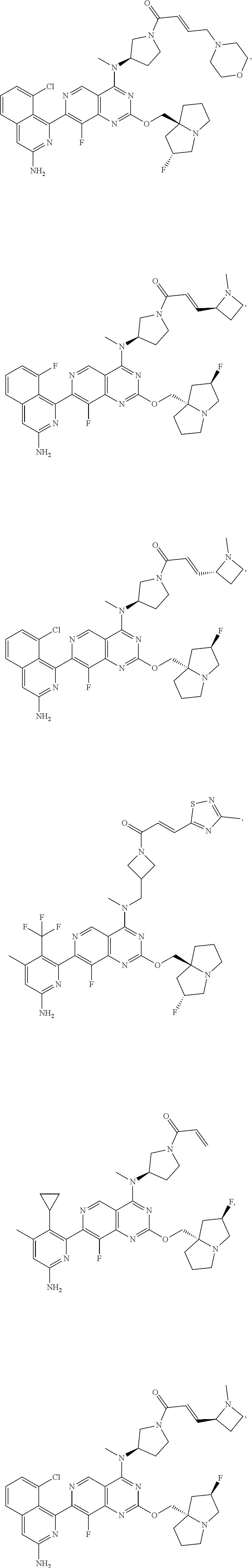

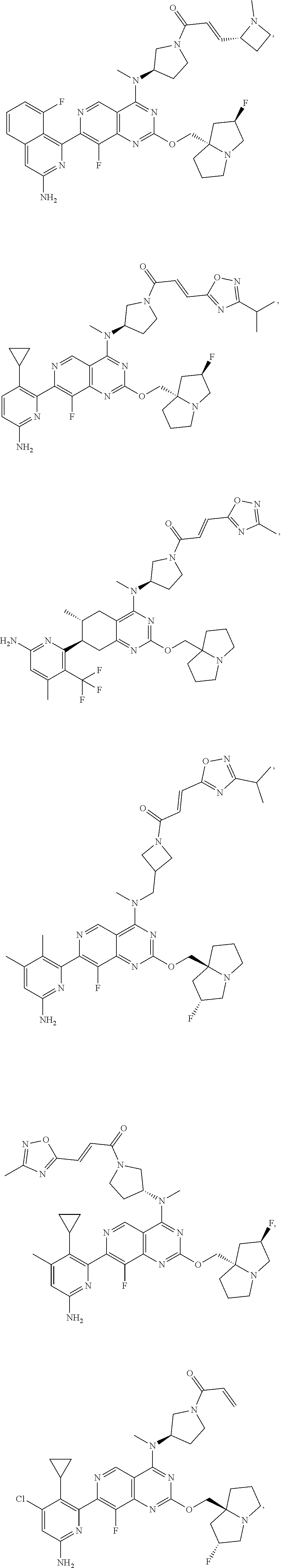

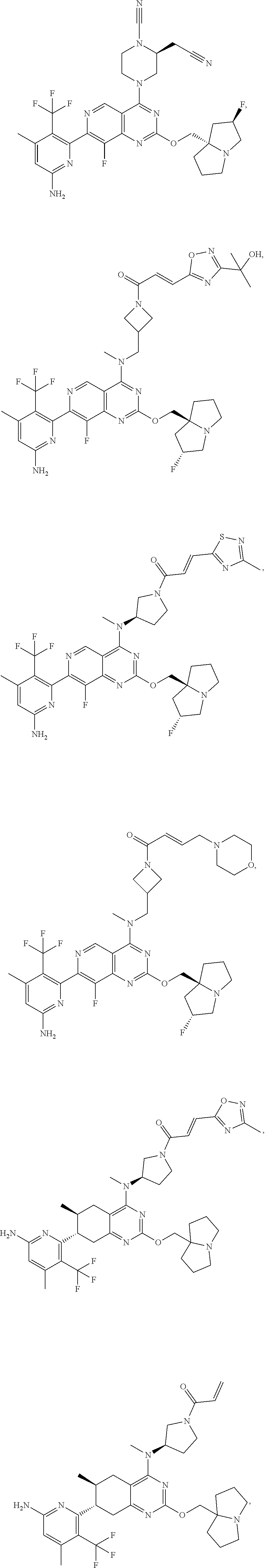

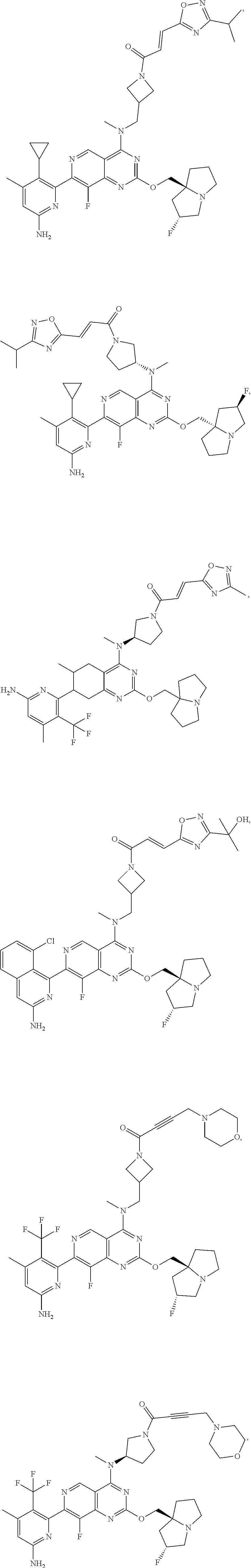

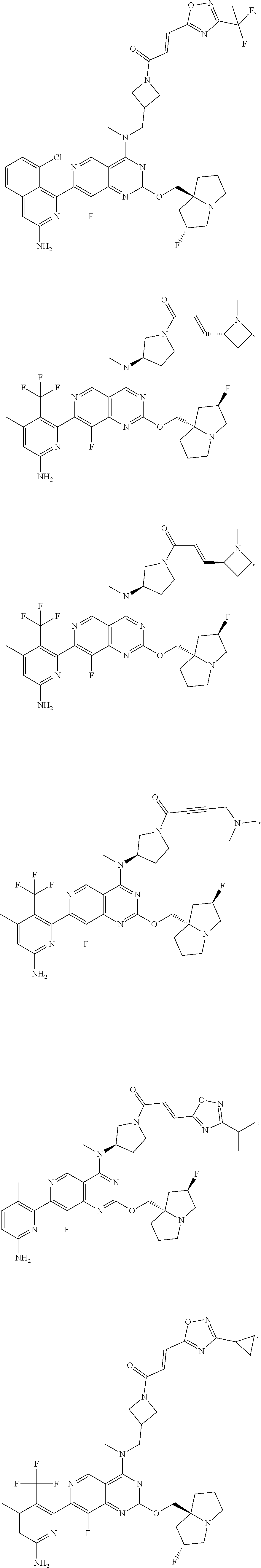

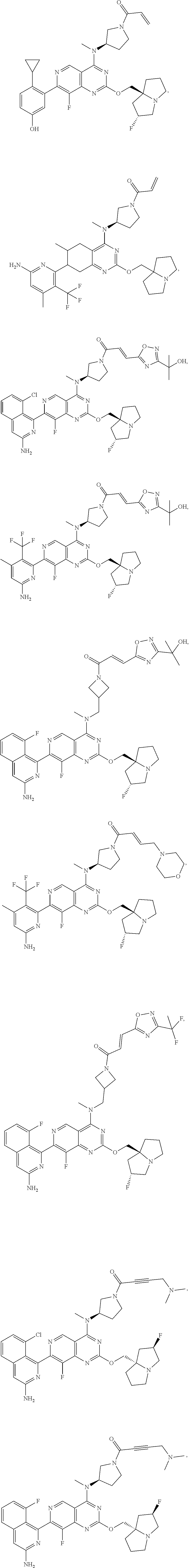

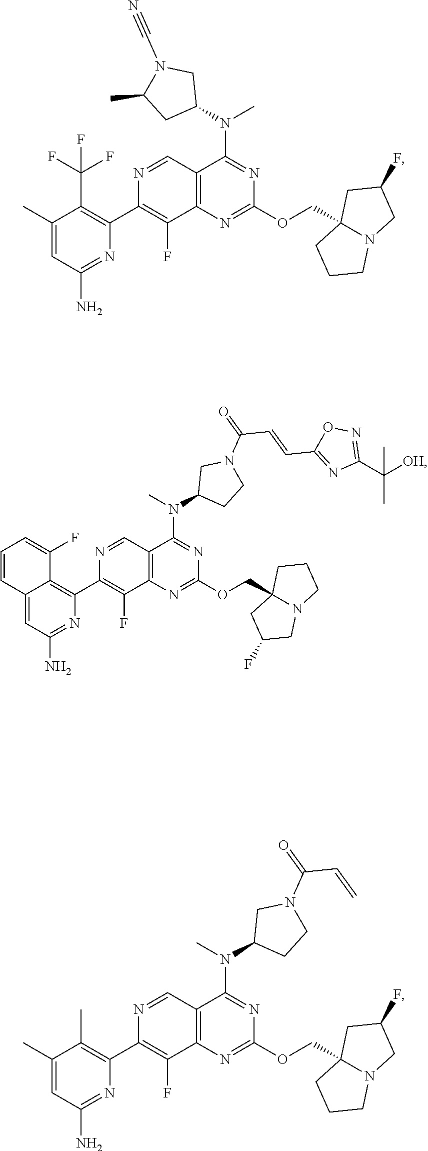 [SMILES: Cc1cc(N)nc(-c2ncc3c(N(C)[C@@H]4C[C@@H](C)N(C#N)C4)nc(OC[C@@]45CCCN4C[C@H](F)C5)nc3c2F)c1C(F)(F)F ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1]

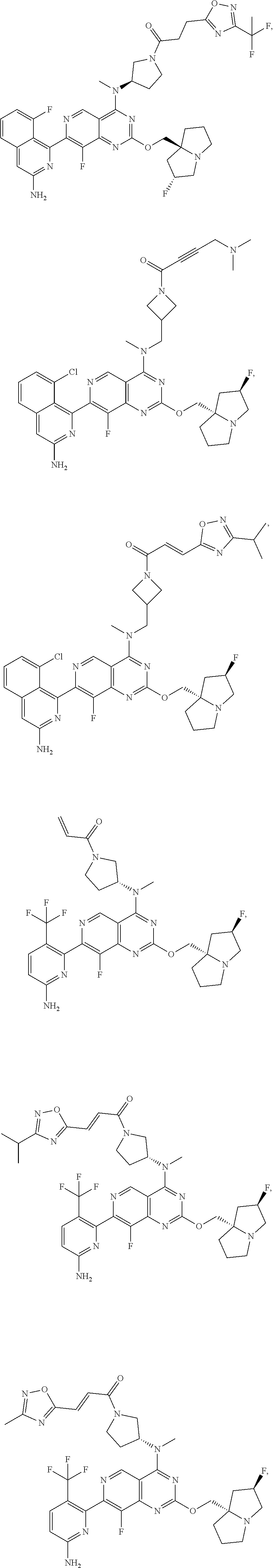

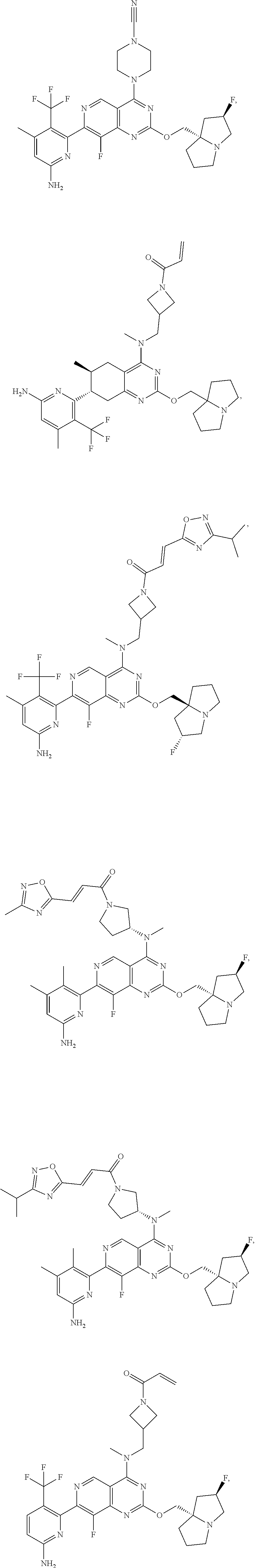

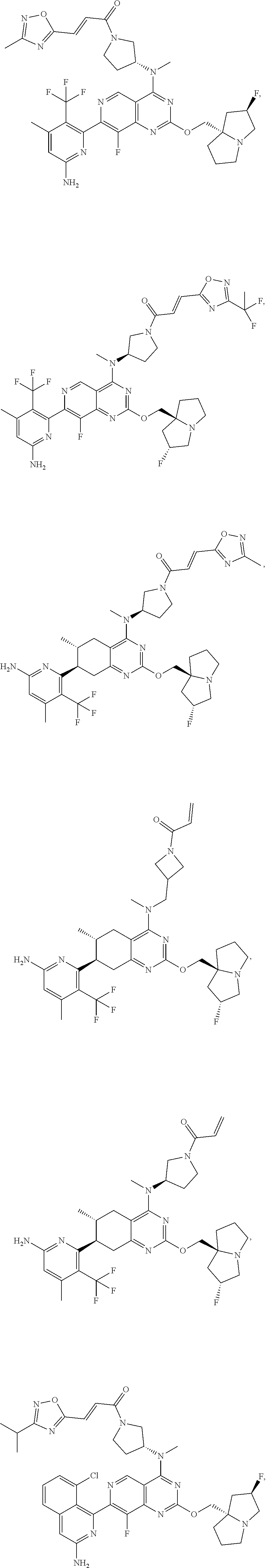

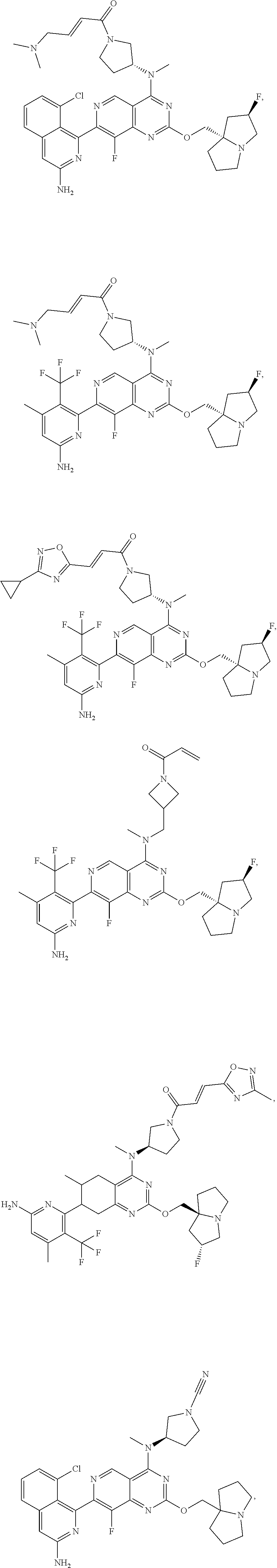

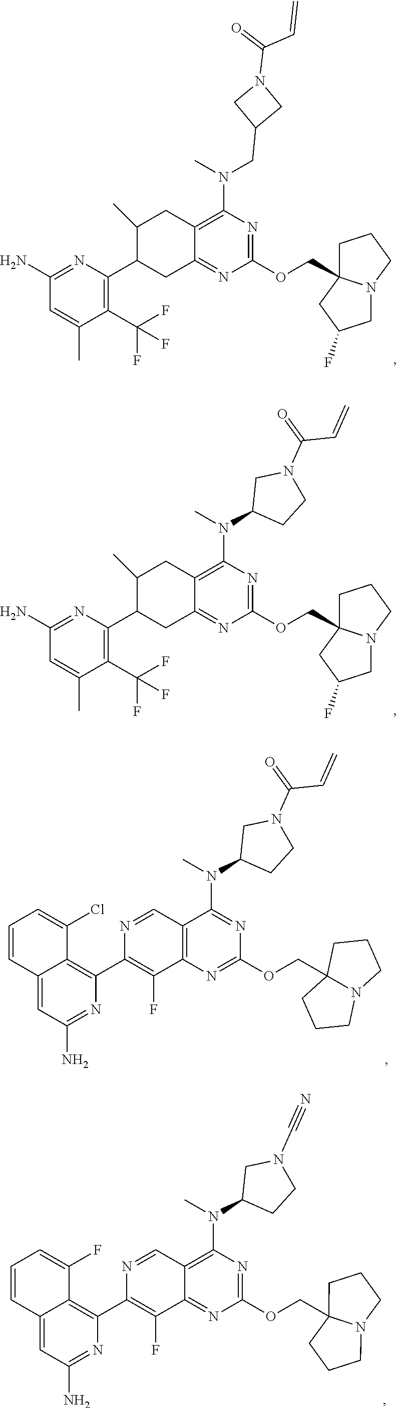

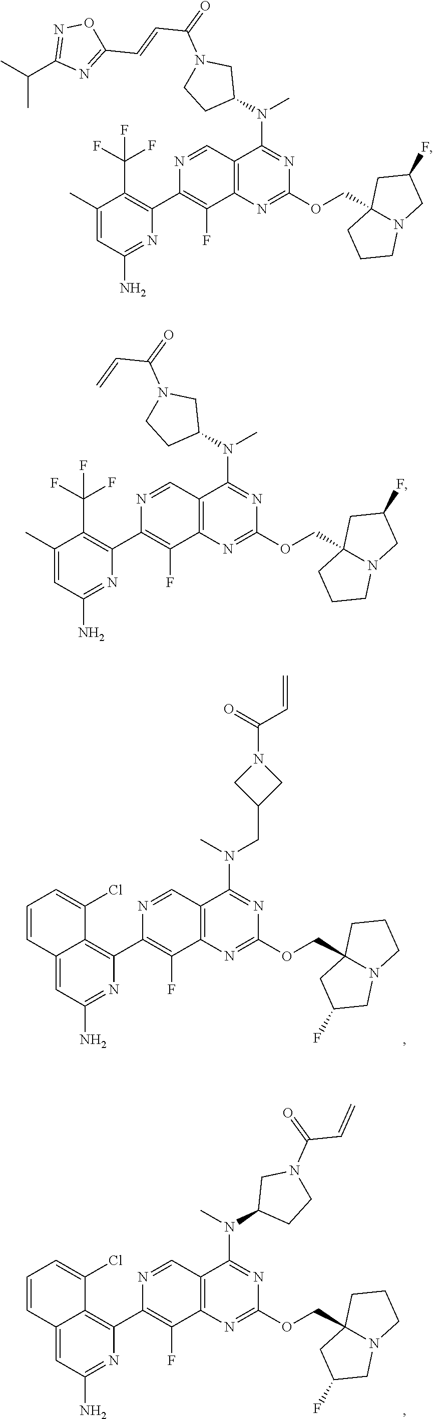 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F ; C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1 ; C=CC(=O)N1CC(CN(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OC[C@@]34CCCN3C[C@H](F)C4)nc3c(F)c(-c4nc(N)cc5cccc(Cl)c45)ncc23)C1]

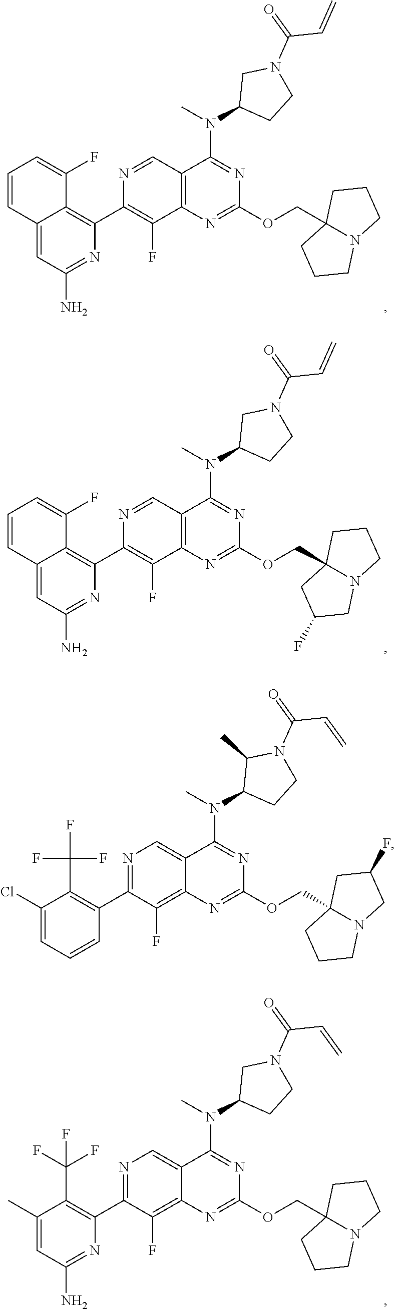 [SMILES: C=CC(=O)N1CC[C@@H](N(C)c2nc(OCC34CCCN3CCC4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OCC34CCCN3CCC4)nc3c(F)c(-c4nc(N)cc(C)c4C(F)(F)F)ncc23)C1]

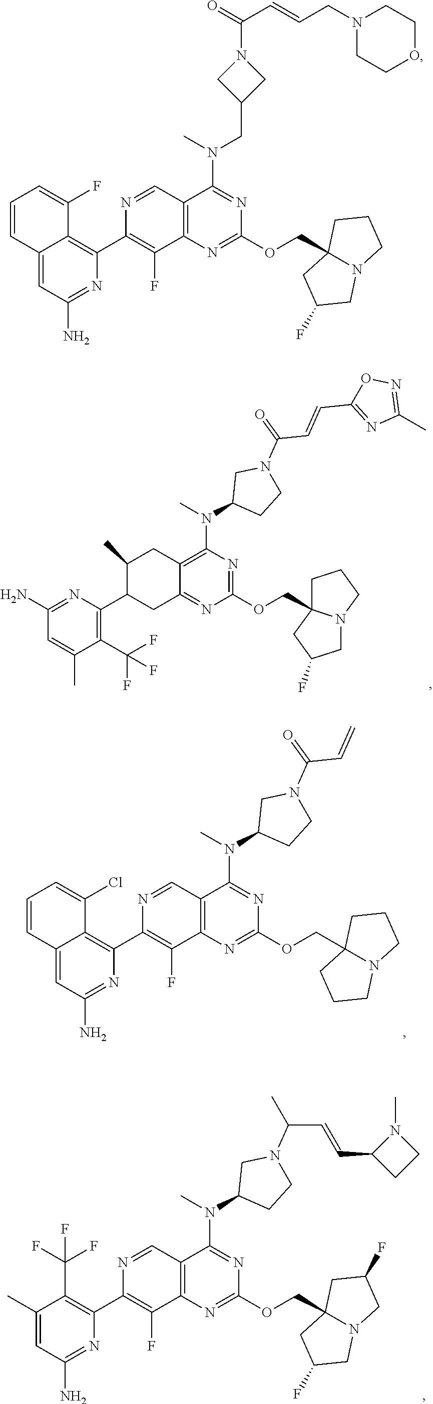

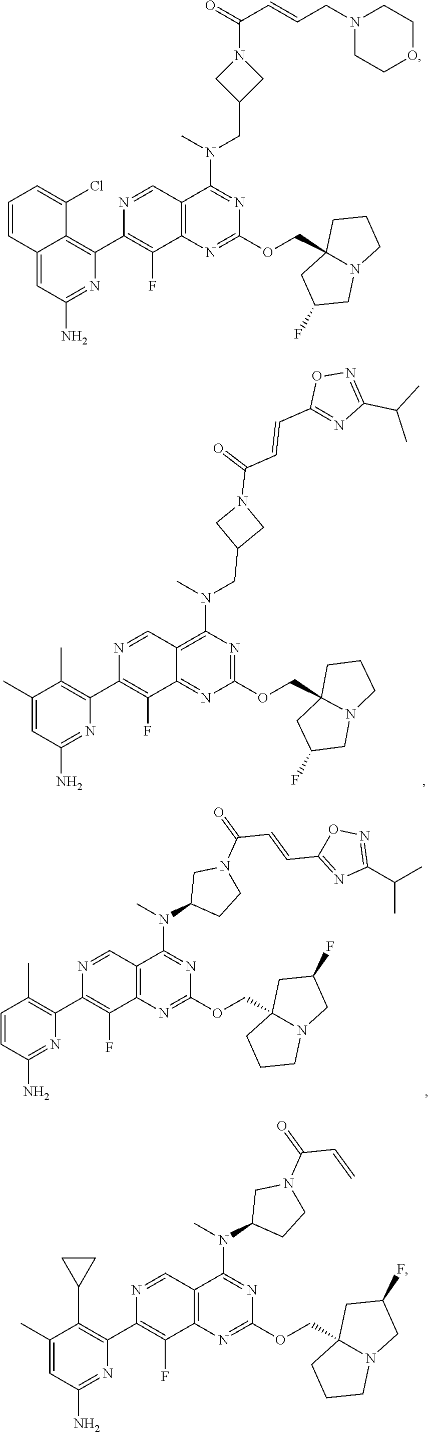

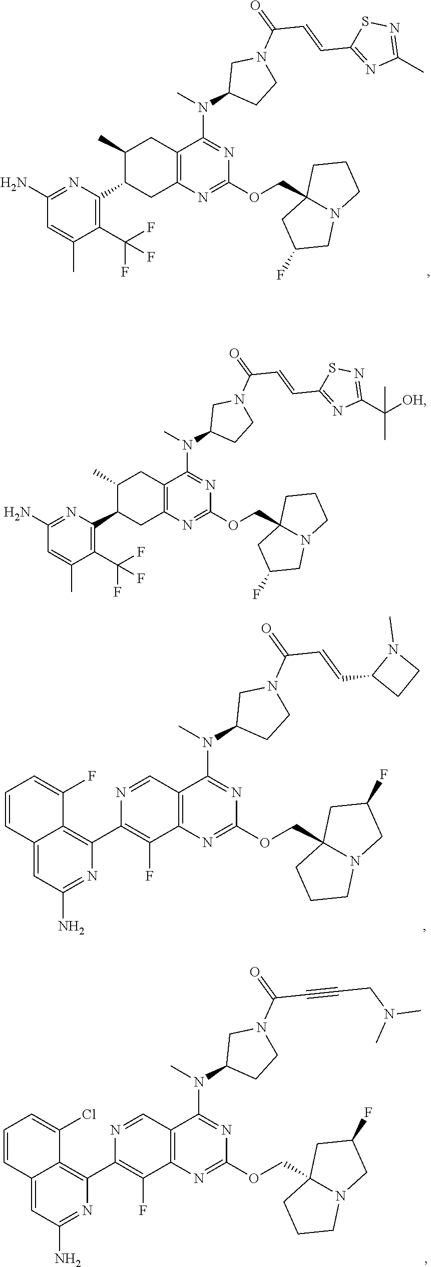

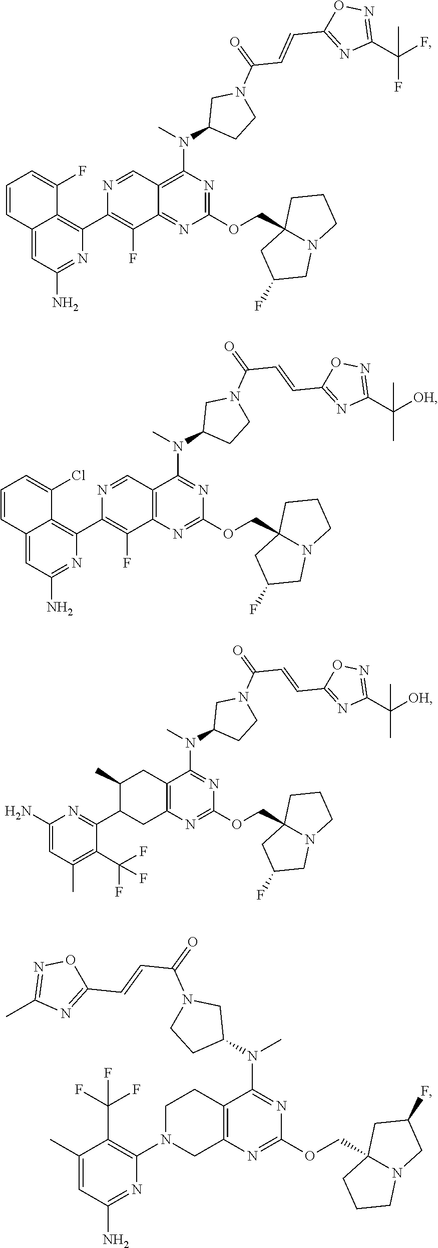 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1 ; CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc(C2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F ; Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1]

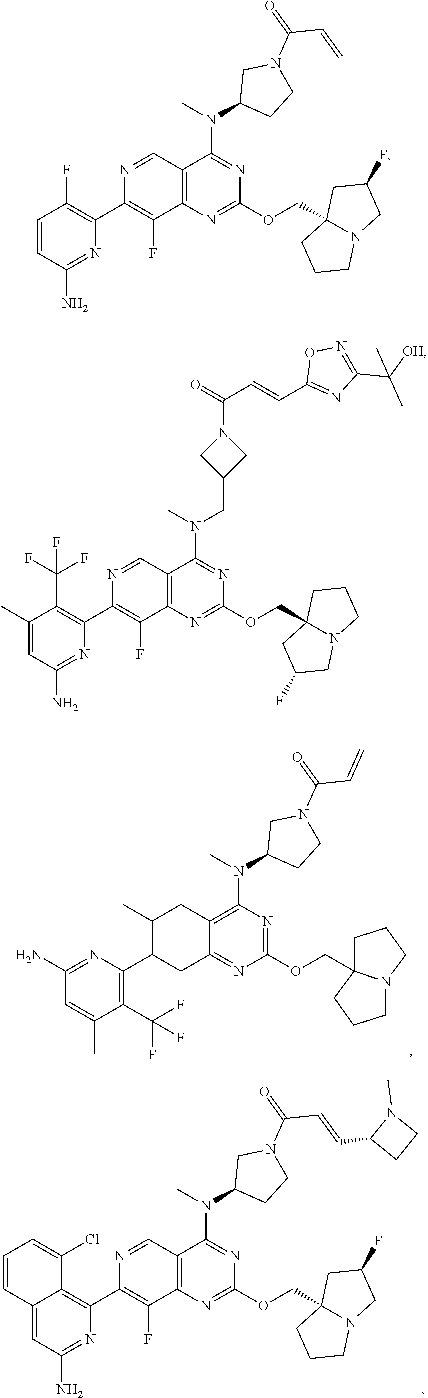 [SMILES: C=CC(=O)N1CC[C@@H](N(C)c2nc(OC[C@@]34CCCN3C[C@H](F)C4)nc3c(F)c(-c4nc(N)ccc4F)ncc23)C1 ; Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F ; C=CC(=O)N1CC[C@@H](N(C)c2nc(OCC34CCCN3CCC4)nc3c2CC(C)C(c2nc(N)cc(C)c2C(F)(F)F)C3)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1]

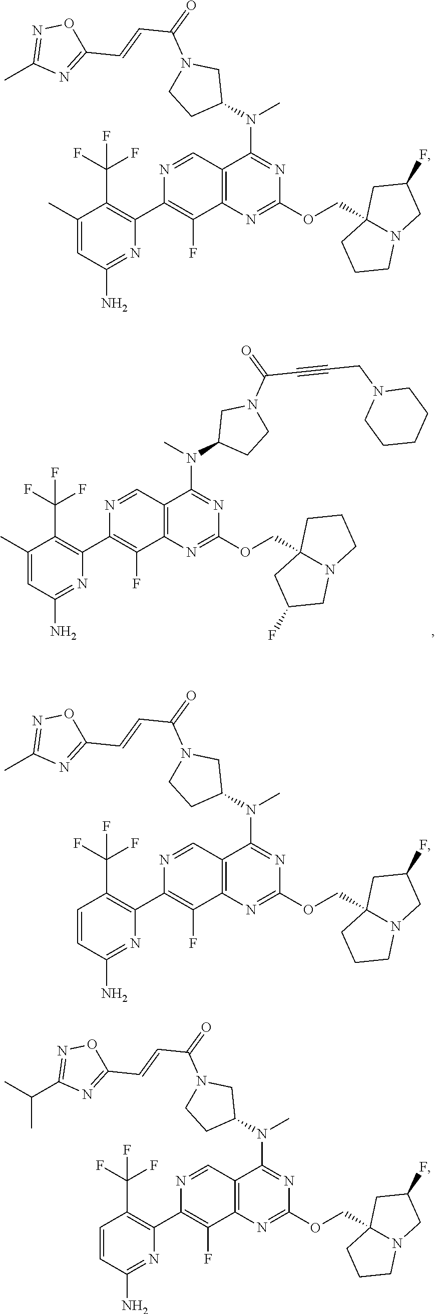 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1 ; Cc1cc(N)nc(-c2ncc3c(N(C)[C@@H]4CCN(C(=O)C#CCN5CCCCC5)C4)nc(OC[C@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1]

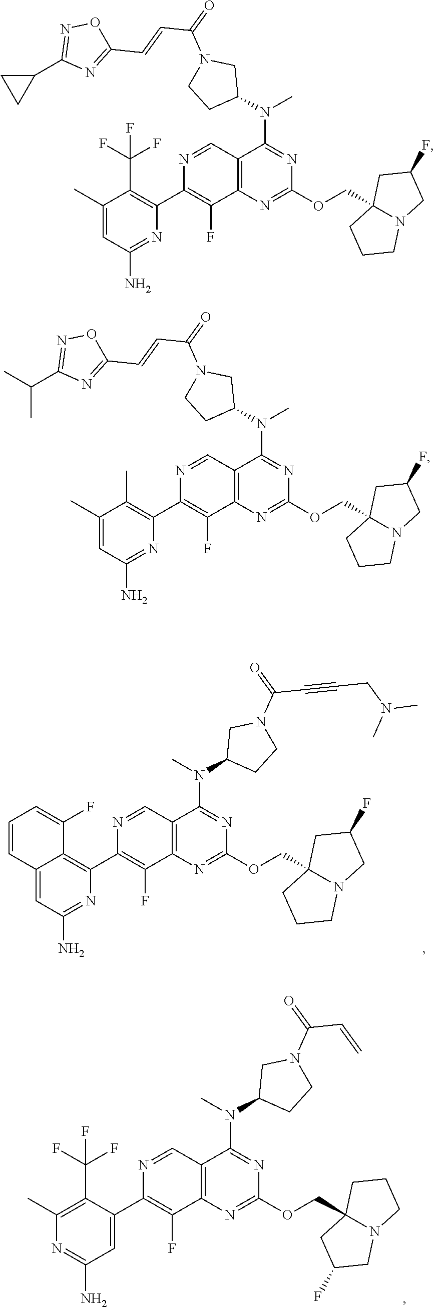

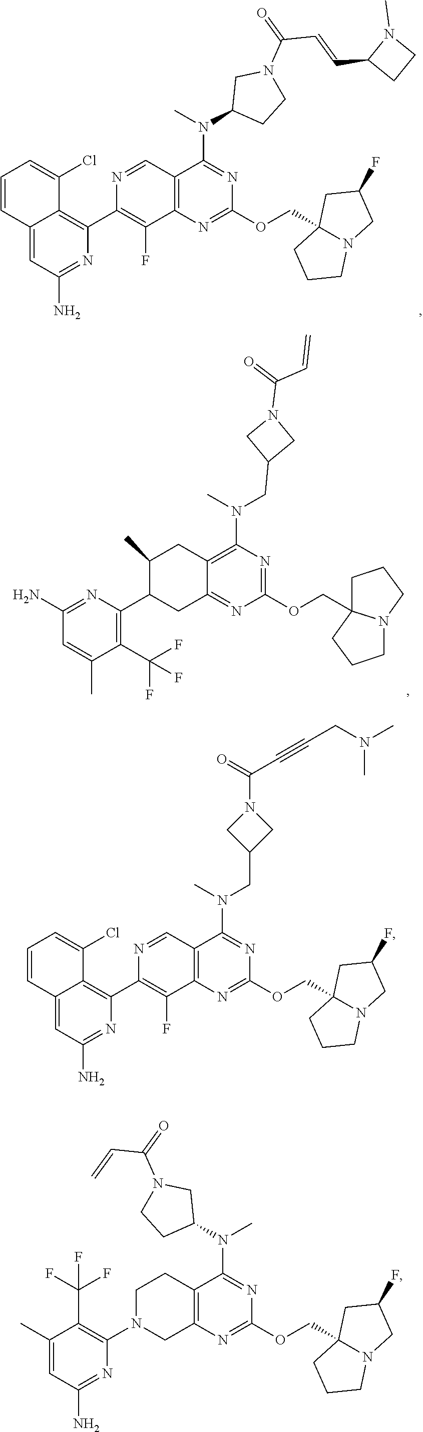

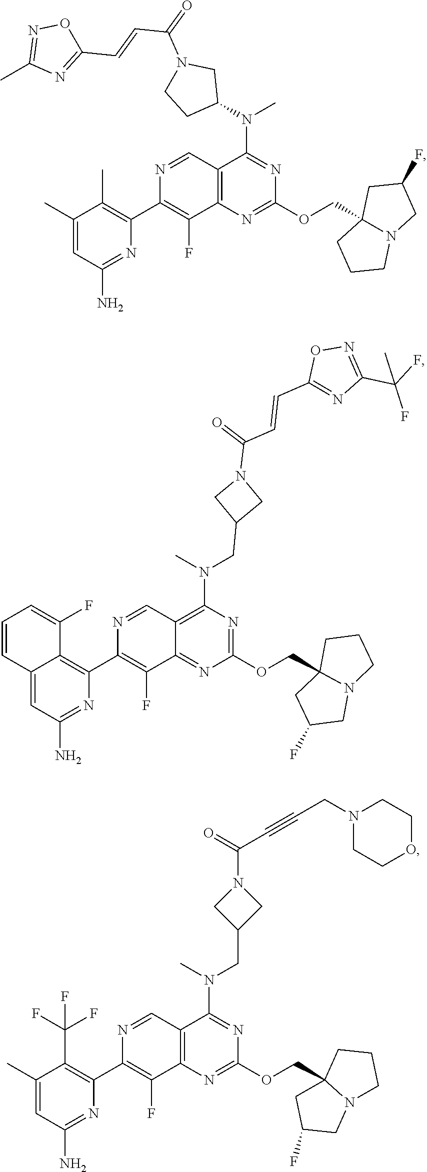 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1 ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12 ; Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F]

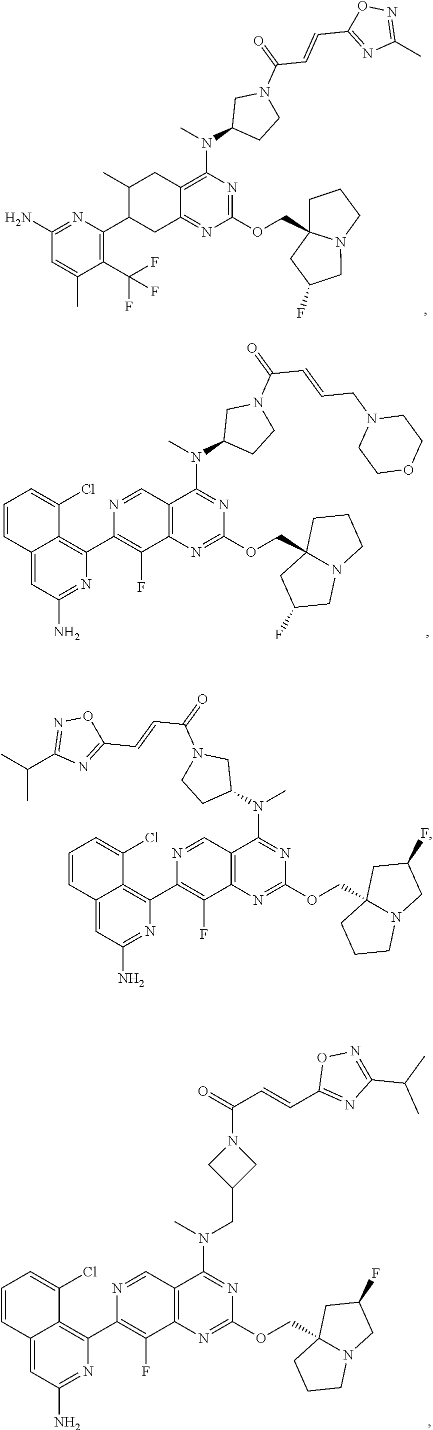

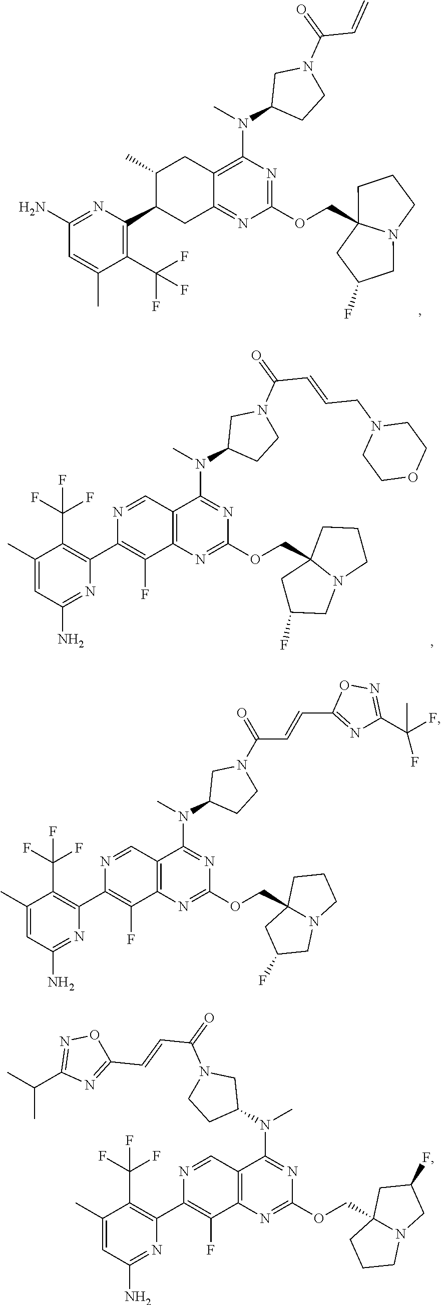 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1 ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F]

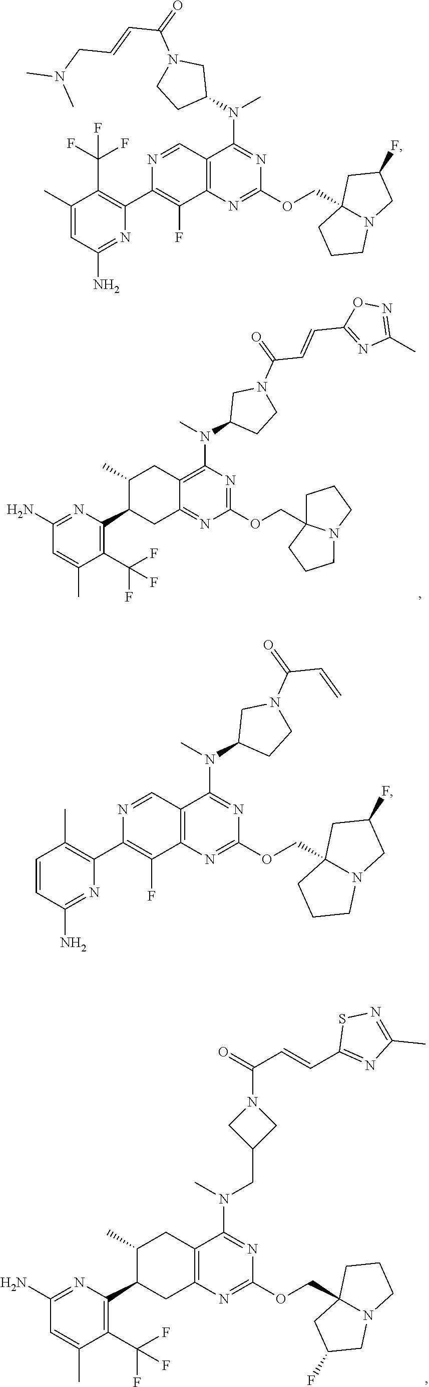 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1 ; C=CC(=O)N1CC[C@@H](N(C)c2nc(OC[C@@]34CCCN3C[C@H](F)C4)nc3c(F)c(-c4nc(N)ccc4C)ncc23)C1 ; Cc1nsc(/C=C/C(=O)N2CC(CN(C)c3nc(OC[C@@]45CCCN4C[C@H](F)C5)nc4c3C[C@@H](C)[C@H](c3nc(N)cc(C)c3C(F)(F)F)C4)C2)n1]

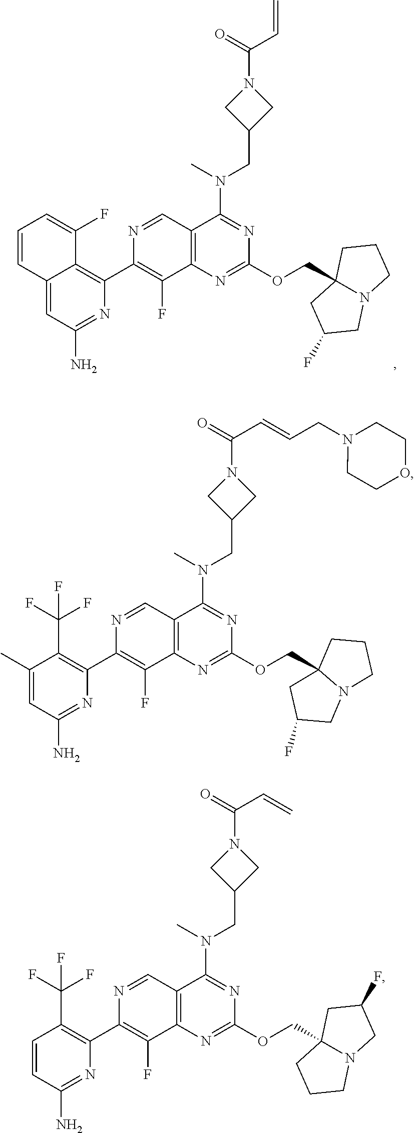 [SMILES: C=CC(=O)N1CC(CN(C)c2nc(OC[C@@]34CCCN3C[C@H](F)C4)nc3c(F)c(-c4nc(N)cc5cccc(F)c45)ncc23)C1 ; Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F ; C=CC(=O)N1CC(CN(C)c2nc(OC[C@@]34CCCN3C[C@H](F)C4)nc3c(F)c(-c4nc(N)ccc4C(F)(F)F)ncc23)C1]

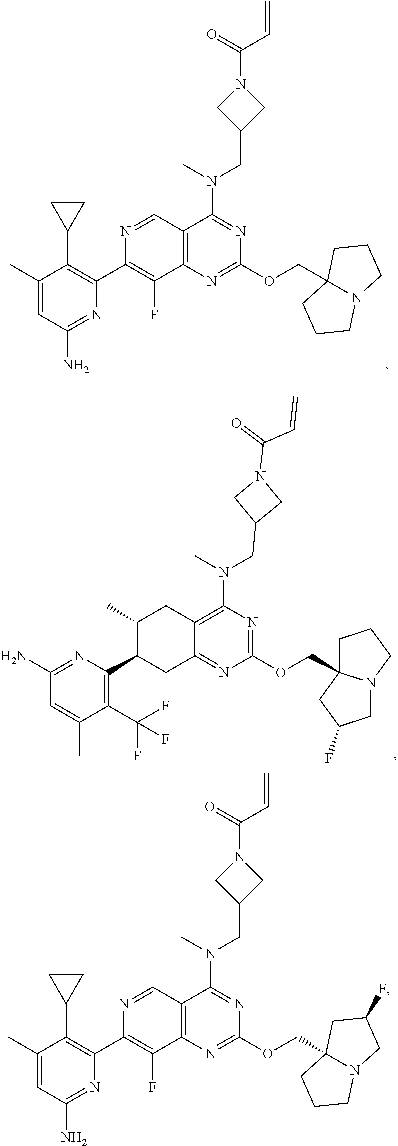 [SMILES: C=CC(=O)N1CC(CN(C)c2nc(OCC34CCCN3CCC4)nc3c(F)c(-c4nc(N)cc(C)c4C4CC4)ncc23)C1 ; C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)cc(C)c4C4CC4)ncc23)C1]

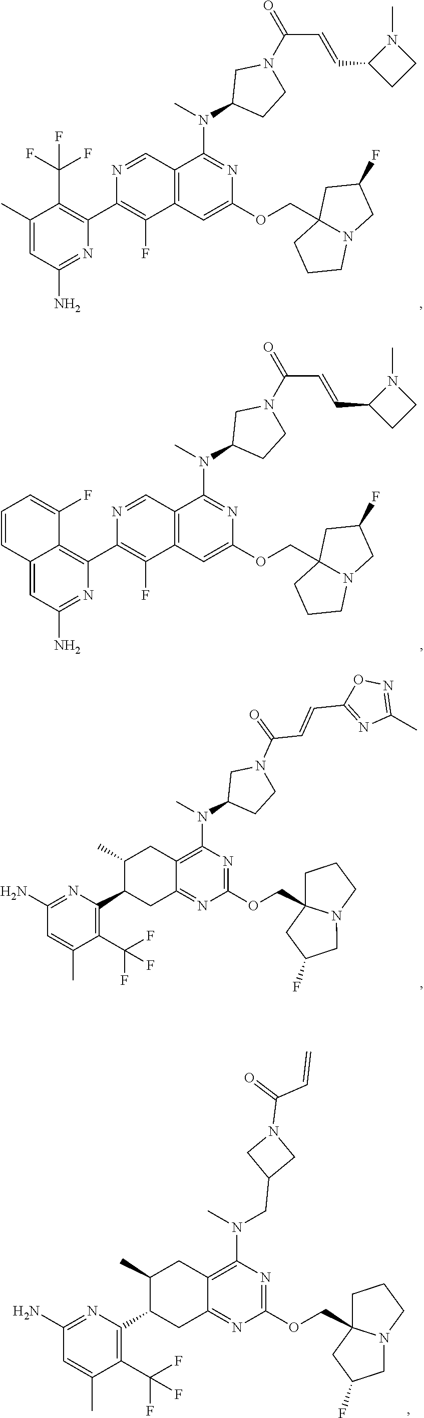 [SMILES: Cc1cc(N)nc(-c2ncc3c(N(C)[C@@H]4CCN(C(=O)/C=C/[C@H]5CCN5C)C4)nc(OCC45CCCN4C[C@H](F)C5)cc3c2F)c1C(F)(F)F ; CN1CC[C@H]1/C=C/C(=O)N1CC[C@@H](N(C)c2nc(OCC34CCCN3C[C@H](F)C4)cc3c(F)c(-c4nc(N)cc5cccc(F)c45)ncc23)C1 ; Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1 ; C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1]

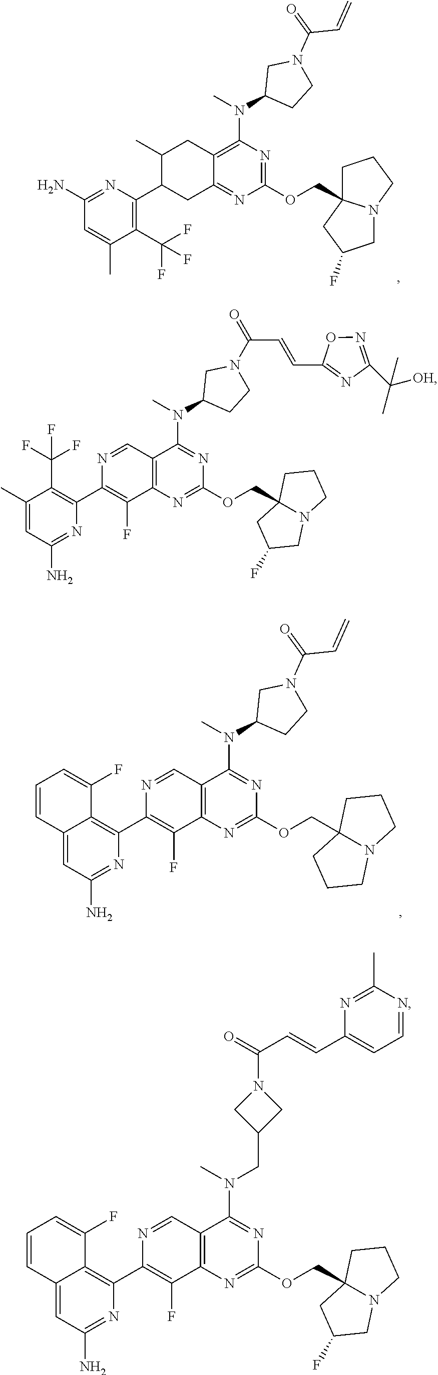 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1 ; Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; C=CC(=O)N1CC[C@@H](N(C)c2nc(OCC34CCCN3CCC4)nc3c(F)c(-c4nc(N)cc5cccc(F)c45)ncc23)C1 ; Cc1nccc(/C=C/C(=O)N2CC(CN(C)c3nc(OC[C@@]45CCCN4C[C@H](F)C5)nc4c(F)c(-c5nc(N)cc6cccc(F)c56)ncc34)C2)n1]

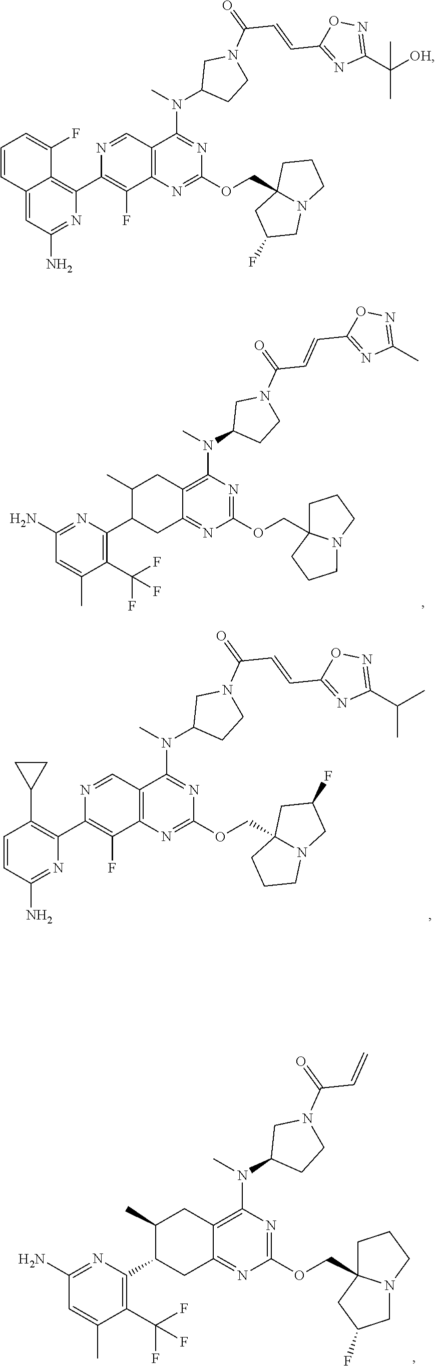 [SMILES: CN(c1nc(OC[C@@]23CCCN2C[C@H](F)C3)nc2c(F)c(-c3nc(N)cc4cccc(F)c34)ncc12)C1CCN(C(=O)/C=C/c2nc(C(C)(C)O)no2)C1 ; Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CC(C)c1noc(/C=C/C(=O)N2CCC(N(C)c3nc(OC[C@@]45CCCN4C[C@H](F)C5)nc4c(F)c(-c5nc(N)ccc5C5CC5)ncc34)C2)n1 ; C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1]

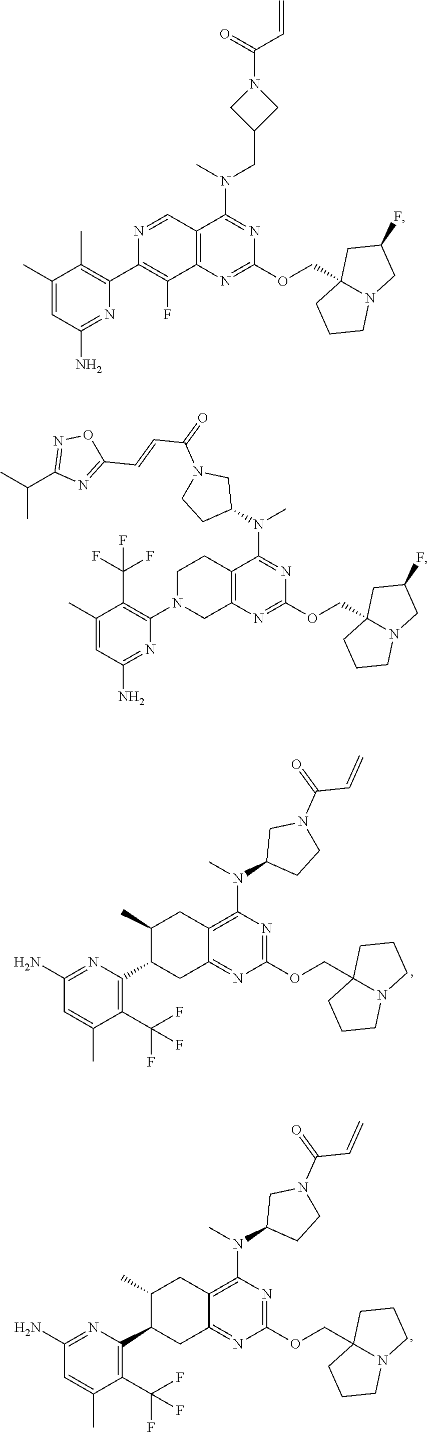 [SMILES: C=CC(=O)N1CC(CN(C)c2nc(OC[C@@]34CCCN3C[C@H](F)C4)nc3c(F)c(-c4nc(N)cc(C)c4C)ncc23)C1 ; Cc1cc(N)nc(N2CCc3c(nc(OC[C@@]45CCCN4C[C@H](F)C5)nc3N(C)[C@@H]3CCN(C(=O)/C=C/c4nc(C(C)C)no4)C3)C2)c1C(F)(F)F ; C=CC(=O)N1CC[C@@H](N(C)c2nc(OCC34CCCN3CCC4)nc3c2C[C@H](C)[C@@H](c2nc(N)cc(C)c2C(F)(F)F)C3)C1 ; C=CC(=O)N1CC[C@@H](N(C)c2nc(OCC34CCCN3CCC4)nc3c2C[C@@H](C)[C@H](c2nc(N)cc(C)c2C(F)(F)F)C3)C1]

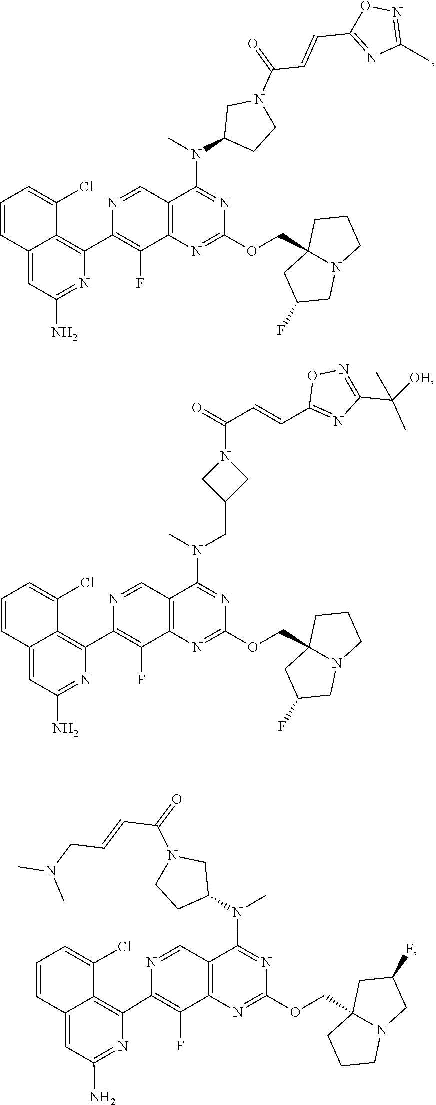

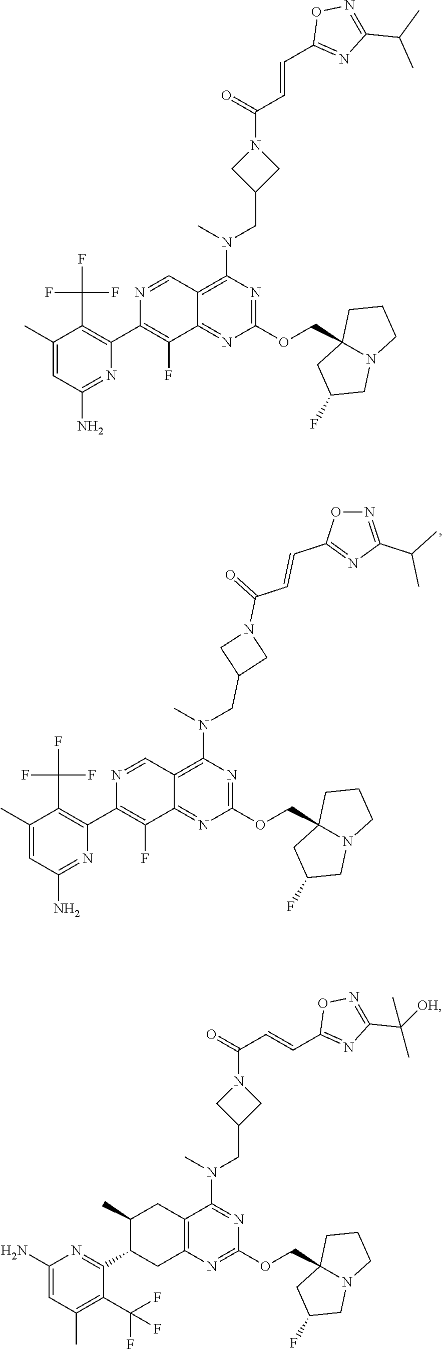

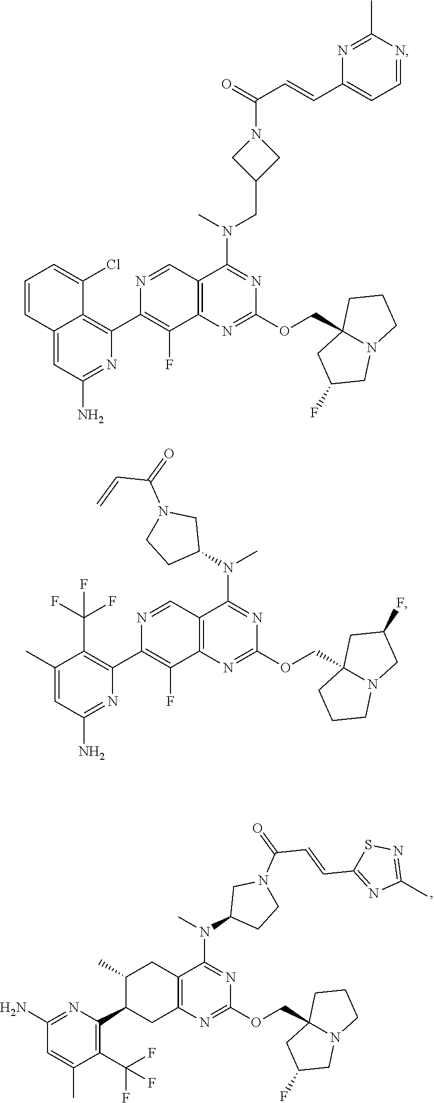 [SMILES: Cc1nccc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1 ; C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1 ; Cc1nsc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1]

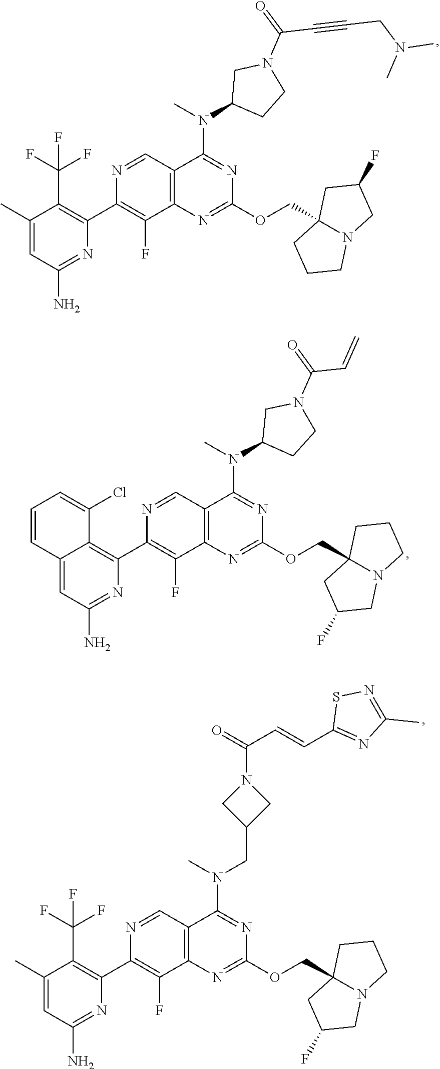

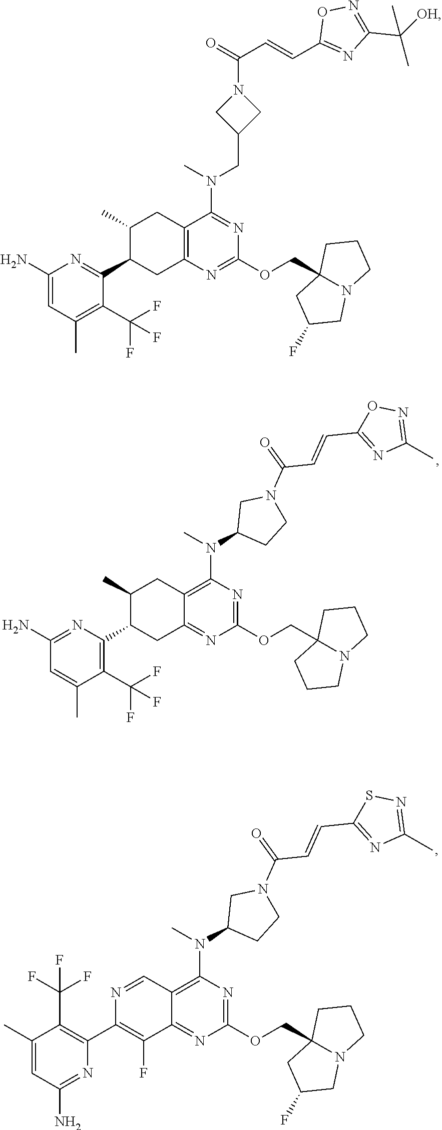

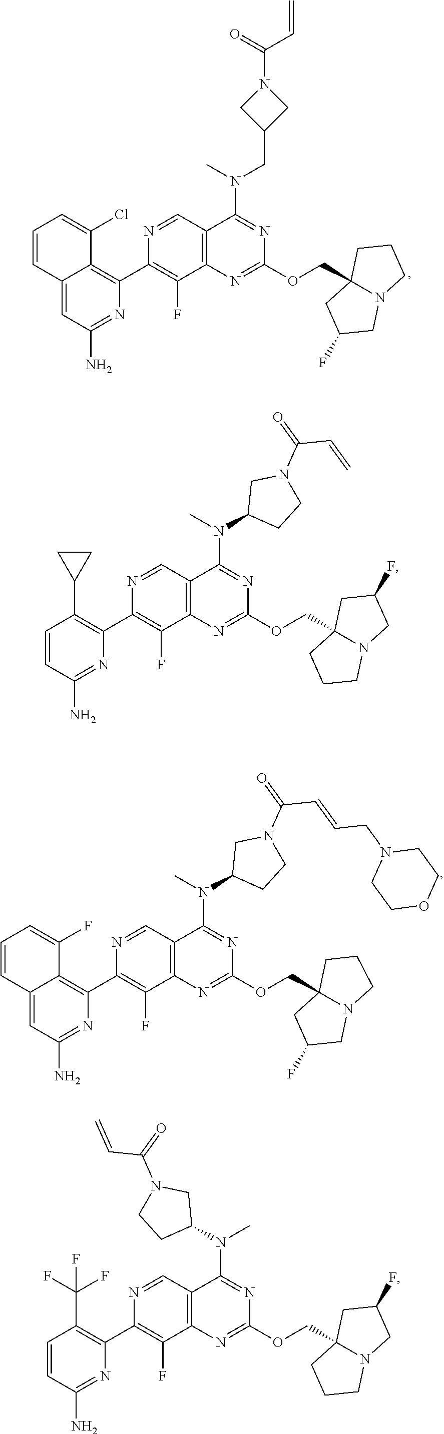 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OC[C@@]34CCCN3C[C@H](F)C4)nc3c(F)c(-c4nc(N)ccc4C4CC4)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1 ; C=CC(=O)N1CC[C@@H](N(C)c2nc(OC[C@@]34CCCN3C[C@H](F)C4)nc3c(F)c(-c4nc(N)ccc4C(F)(F)F)ncc23)C1]

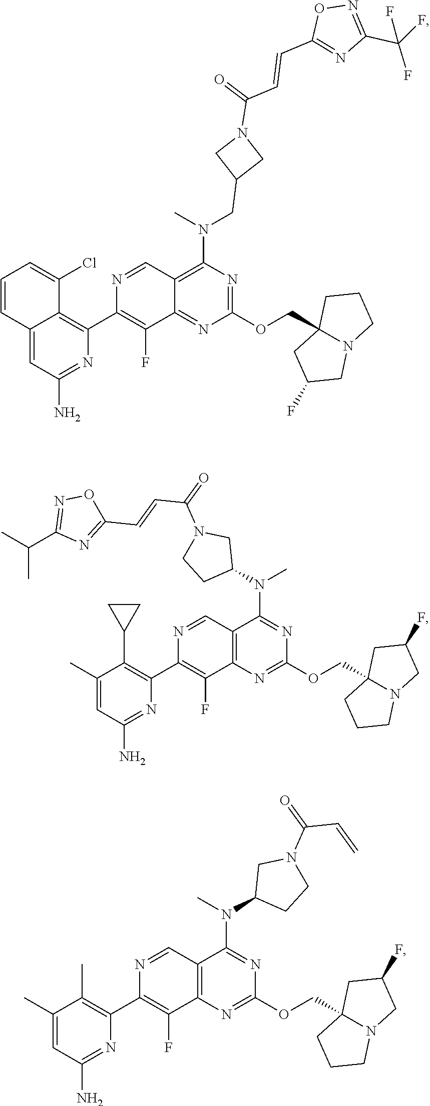 [SMILES: CN(CC1CN(C(=O)/C=C/c2nc(C(F)(F)F)no2)C1)c1nc(OC[C@@]23CCCN2C[C@H](F)C3)nc2c(F)c(-c3nc(N)cc4cccc(Cl)c34)ncc12 ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1 ; C=CC(=O)N1CC[C@@H](N(C)c2nc(OC[C@@]34CCCN3C[C@H](F)C4)nc3c(F)c(-c4nc(N)cc(C)c4C)ncc23)C1]

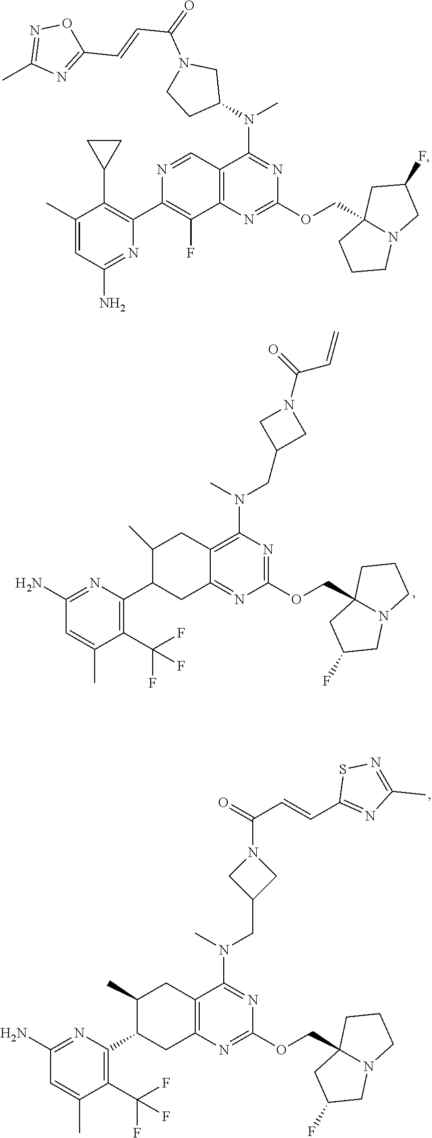 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1 ; C=CC(=O)N1CC(CN(C)c2nc(OC[C@@]34CCCN3C[C@H](F)C4)nc3c2CC(C)C(c2nc(N)cc(C)c2C(F)(F)F)C3)C1 ; Cc1nsc(/C=C/C(=O)N2CC(CN(C)c3nc(OC[C@@]45CCCN4C[C@H](F)C5)nc4c3C[C@H](C)[C@@H](c3nc(N)cc(C)c3C(F)(F)F)C4)C2)n1]

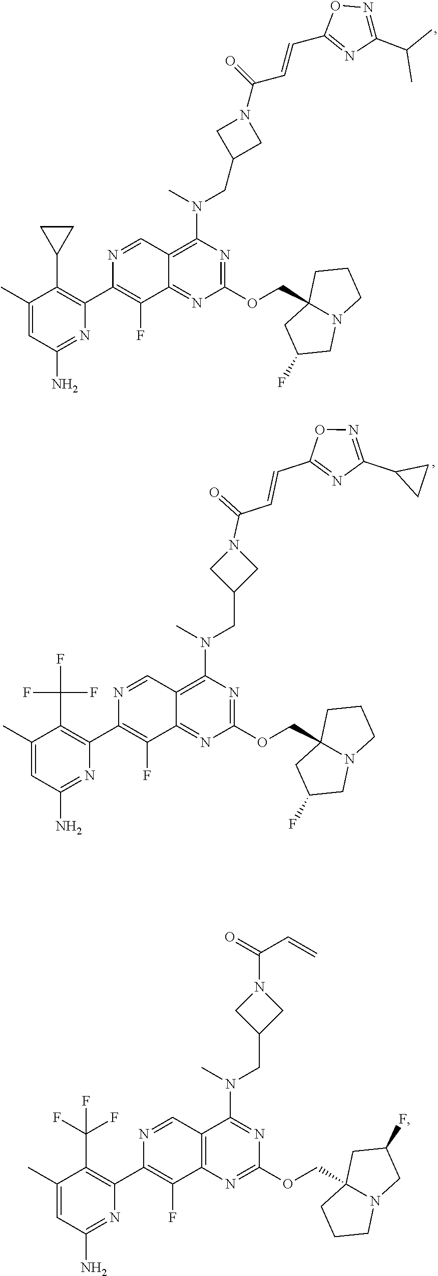

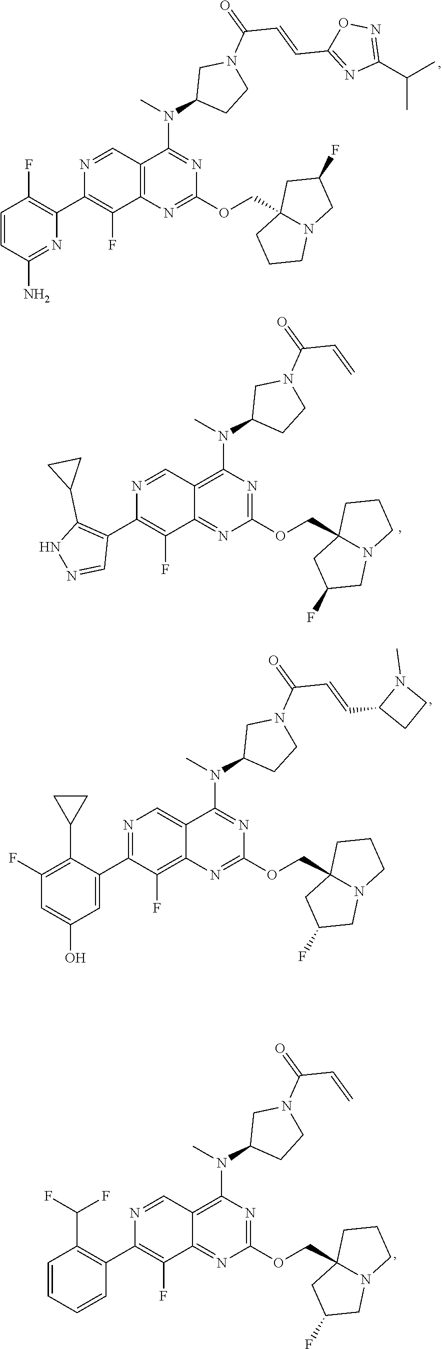

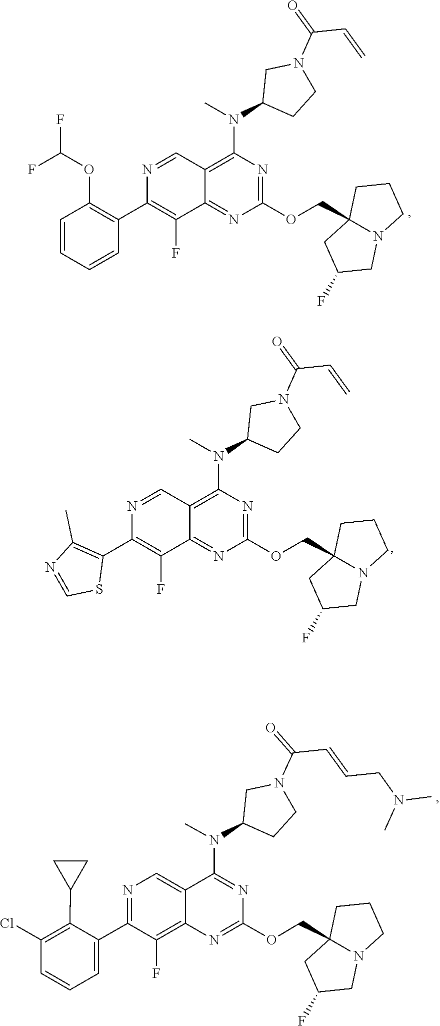 [SMILES: C=CC(=O)N1CC[C@@H](N(C)c2nc(OC[C@@]34CCCN3C[C@H](F)C4)nc3c(F)c(-c4ccccc4OC(F)F)ncc23)C1 ; C=CC(=O)N1CC[C@@H](N(C)c2nc(OC[C@@]34CCCN3C[C@H](F)C4)nc3c(F)c(-c4scnc4C)ncc23)C1 ; CN(C)C/C=C/C(=O)N1CC[C@@H](N(C)c2nc(OC[C@@]34CCCN3C[C@H](F)C4)nc3c(F)c(-c4cccc(Cl)c4C4CC4)ncc23)C1]

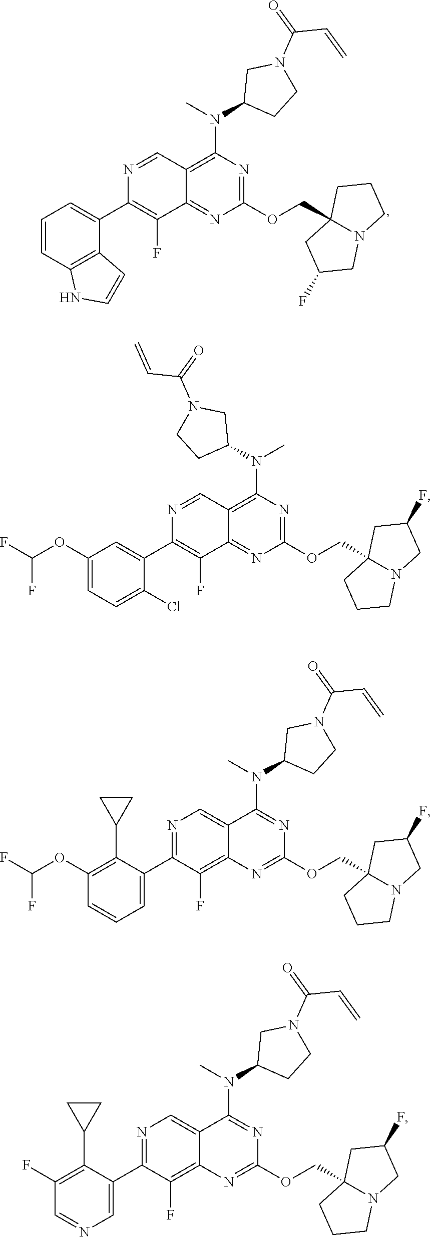 [SMILES: C=CC(=O)N1CC[C@@H](N(C)c2nc(OC[C@@]34CCCN3C[C@H](F)C4)nc3c(F)c(-c4cccc5[nH]ccc45)ncc23)C1 ; C=CC(=O)N1CC[C@@H](N(C)c2nc(OC[C@@]34CCCN3C[C@H](F)C4)nc3c(F)c(-c4cc(OC(F)F)ccc4Cl)ncc23)C1 ; C=CC(=O)N1CC[C@@H](N(C)c2nc(OC[C@@]34CCCN3C[C@H](F)C4)nc3c(F)c(-c4cccc(OC(F)F)c4C4CC4)ncc23)C1 ; C=CC(=O)N1CC[C@@H](N(C)c2nc(OC[C@@]34CCCN3C[C@H](F)C4)nc3c(F)c(-c4cncc(F)c4C4CC4)ncc23)C1]

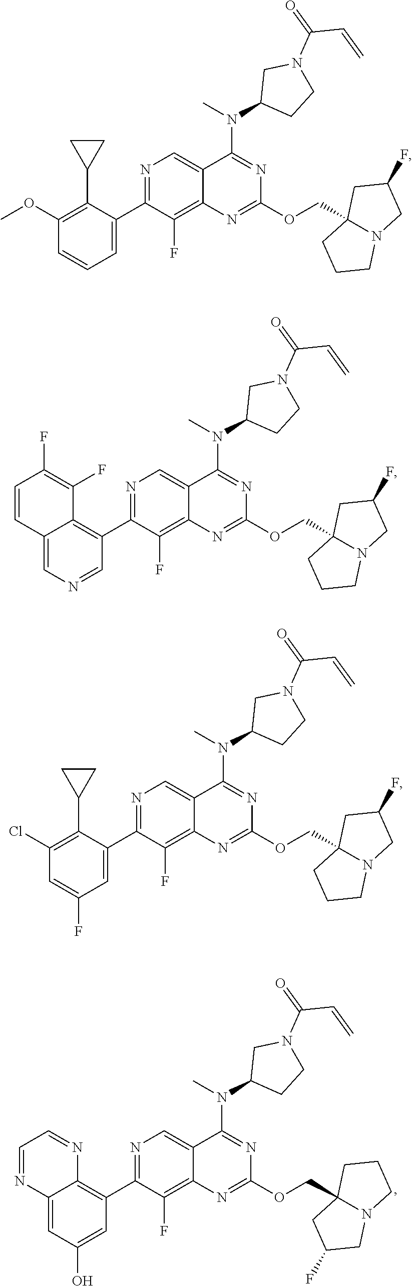 [SMILES: C=CC(=O)N1CC[C@@H](N(C)c2nc(OC[C@@]34CCCN3C[C@H](F)C4)nc3c(F)c(-c4cccc(OC)c4C4CC4)ncc23)C1 ; C=CC(=O)N1CC[C@@H](N(C)c2nc(OC[C@@]34CCCN3C[C@H](F)C4)nc3c(F)c(-c4cncc5ccc(F)c(F)c45)ncc23)C1 ; C=CC(=O)N1CC[C@@H](N(C)c2nc(OC[C@@]34CCCN3C[C@H](F)C4)nc3c(F)c(-c4cc(F)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cc(O)cc5nccnc45)ncc23)C1]

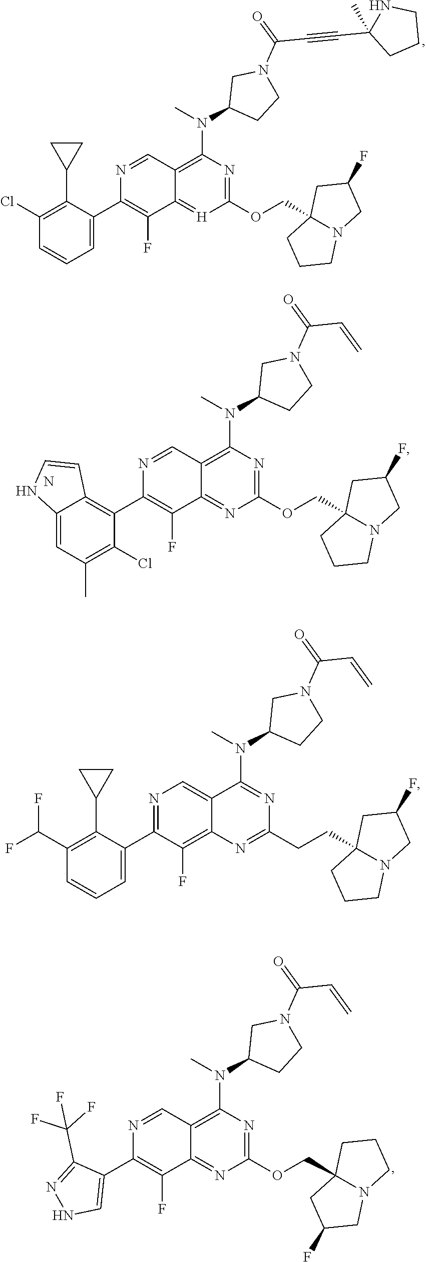

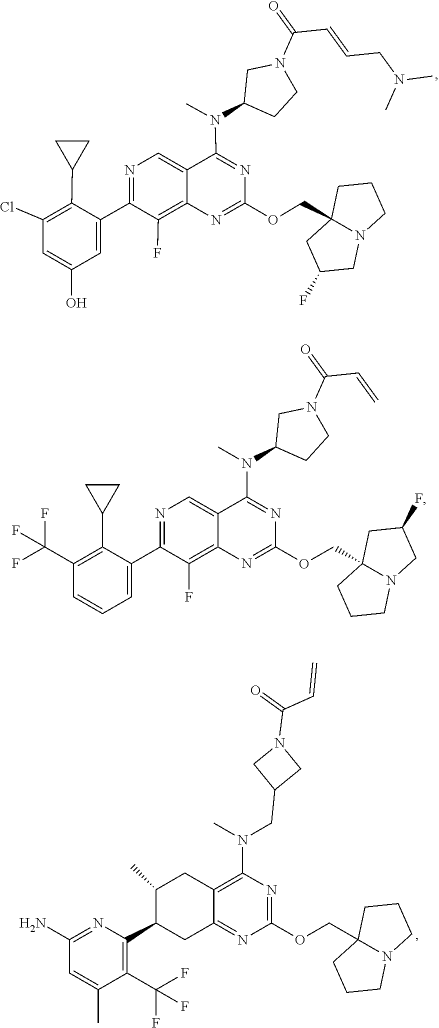 [SMILES: CN(C)C/C=C/C(=O)N1CC[C@@H](N(C)c2nc(OC[C@@]34CCCN3C[C@H](F)C4)nc3c(F)c(-c4cc(O)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cccc(C(F)(F)F)c4C4CC4)ncc23)C1 ; C=CC(=O)N1CC(CN(C)c2nc(OCC34CCCN3CCC4)nc3c2C[C@@H](C)[C@H](c2nc(N)cc(C)c2C(F)(F)F)C3)C1]

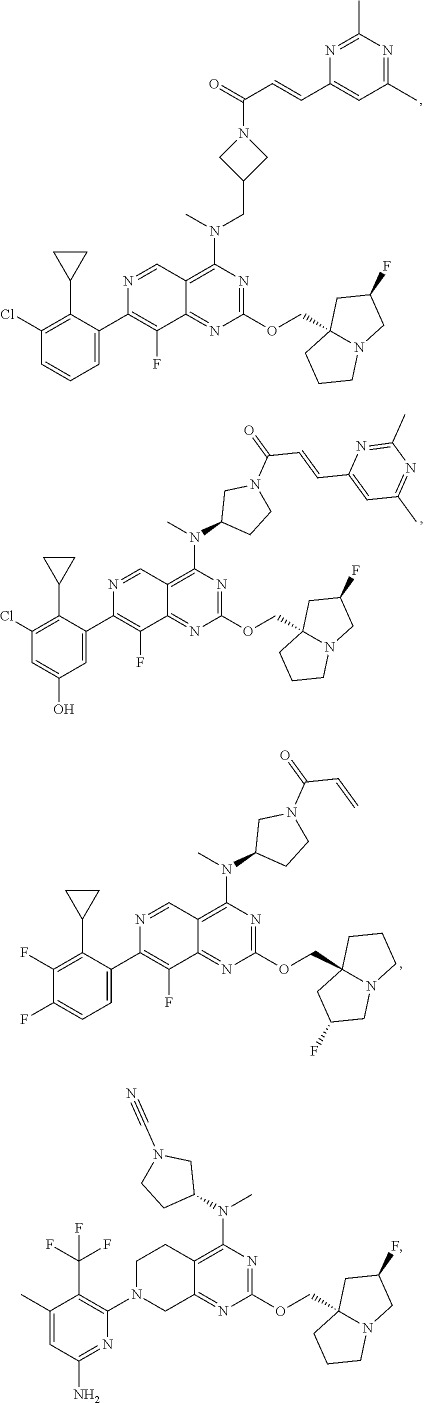

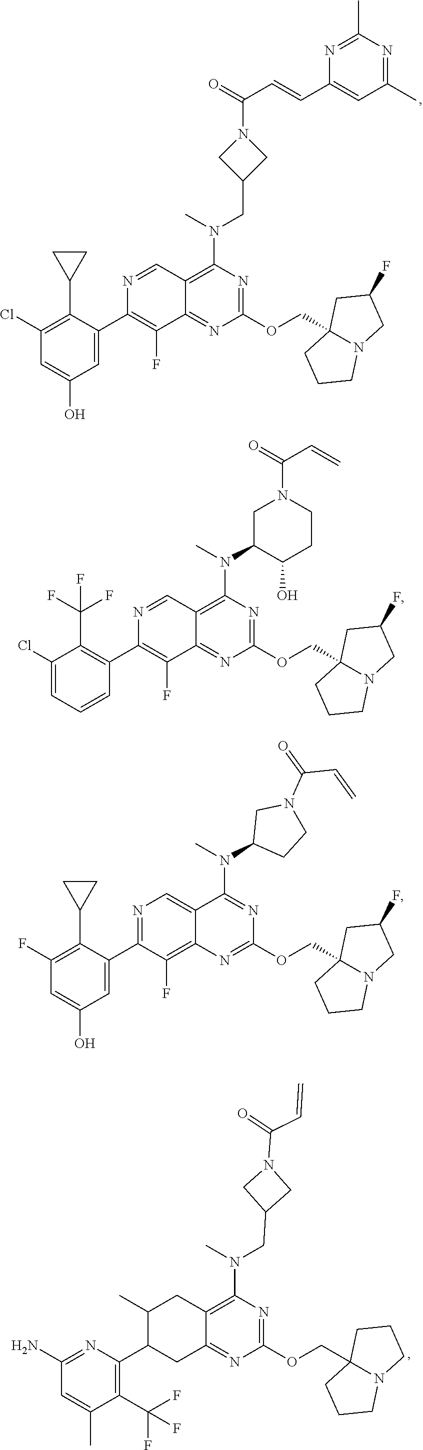 [SMILES: Cc1cc(/C=C/C(=O)N2CC(CN(C)c3nc(OC[C@@]45CCCN4C[C@H](F)C5)nc4c(F)c(-c5cc(O)cc(Cl)c5C5CC5)ncc34)C2)nc(C)n1 ; C=CC(=O)N1CC[C@H](O)[C@@H](N(C)c2nc(OC[C@@]34CCCN3C[C@H](F)C4)nc3c(F)c(-c4cccc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(O)cc(F)c4C4CC4)ncc23)C1 ; C=CC(=O)N1CC(CN(C)c2nc(OCC34CCCN3CCC4)nc3c2CC(C)C(c2nc(N)cc(C)c2C(F)(F)F)C3)C1]

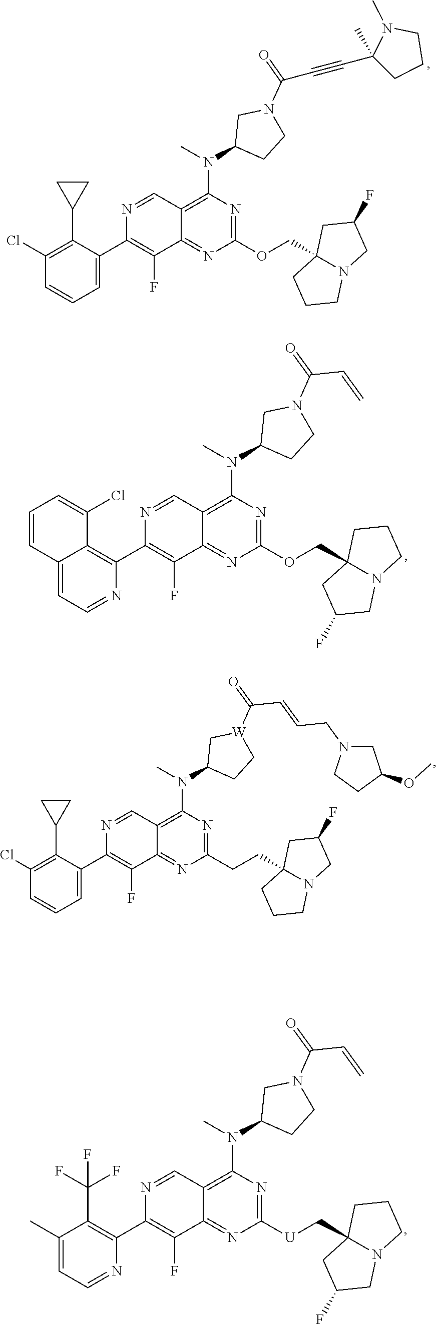

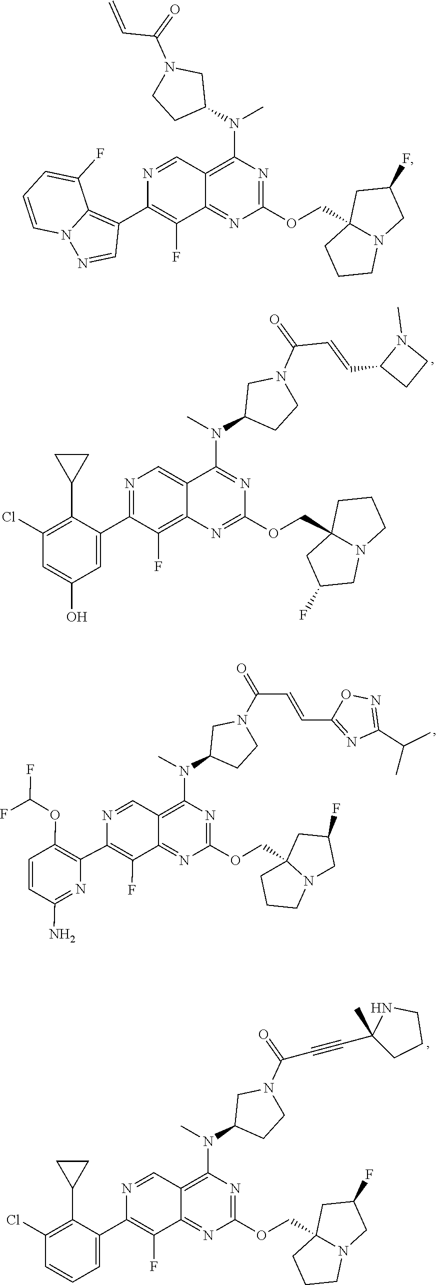

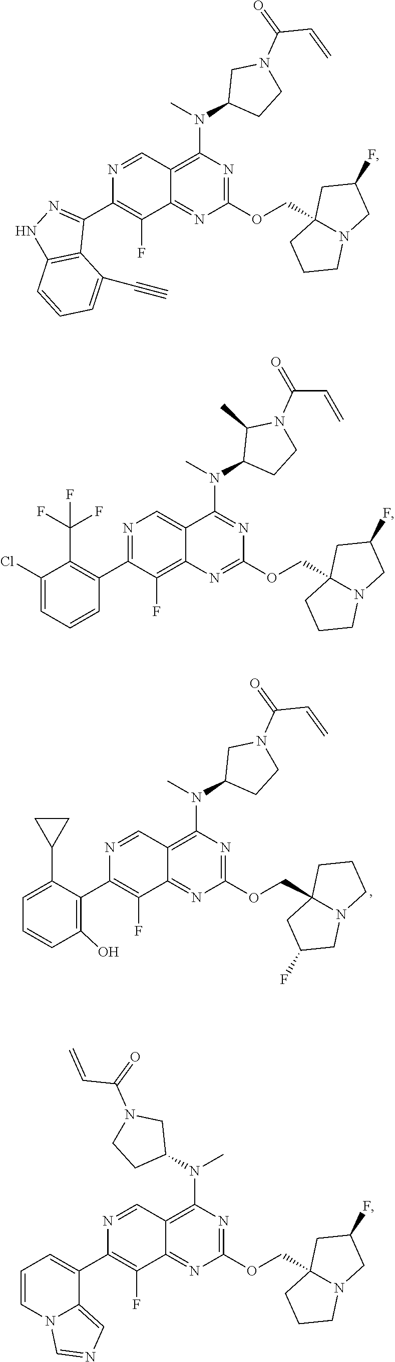 [SMILES: C#Cc1cccc2[nH]nc(-c3ncc4c(N(C)[C@@H]5CCN(C(=O)C=C)C5)nc(OC[C@@]56CCCN5C[C@H](F)C6)nc4c3F)c12 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OC[C@@]34CCCN3C[C@H](F)C4)nc3c(F)c(-c4c(O)cccc4C4CC4)ncc23)C1 ; C=CC(=O)N1CC[C@@H](N(C)c2nc(OC[C@@]34CCCN3C[C@H](F)C4)nc3c(F)c(-c4cccn5cncc45)ncc23)C1]

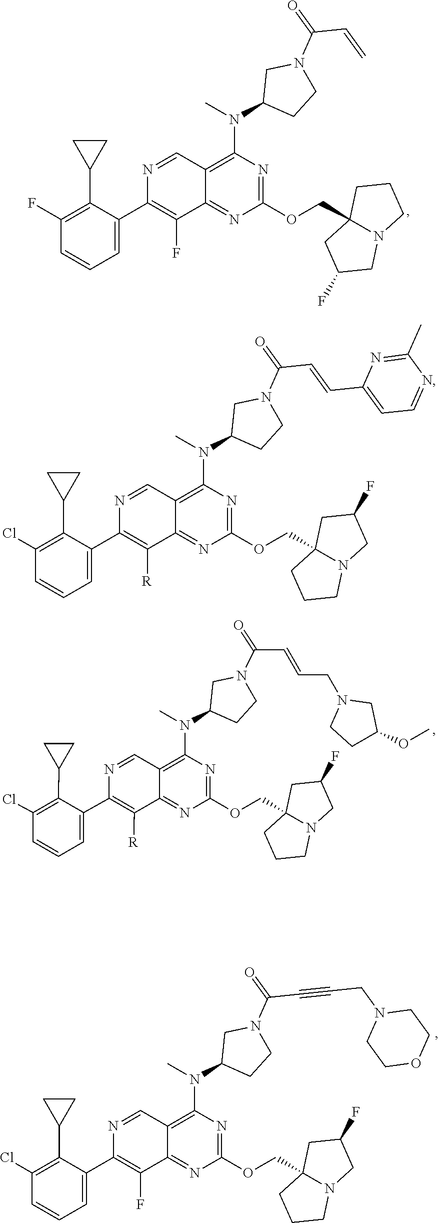

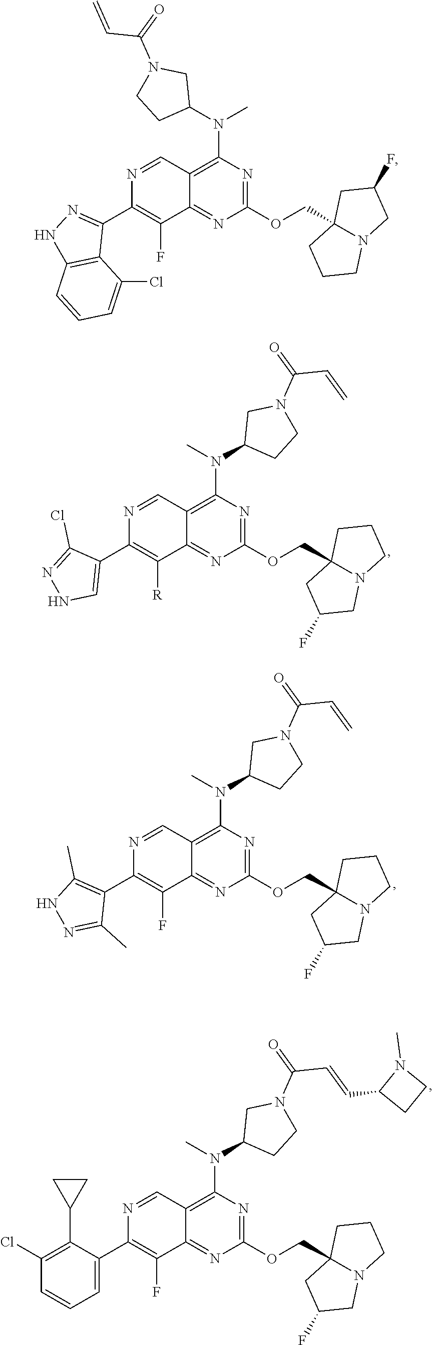

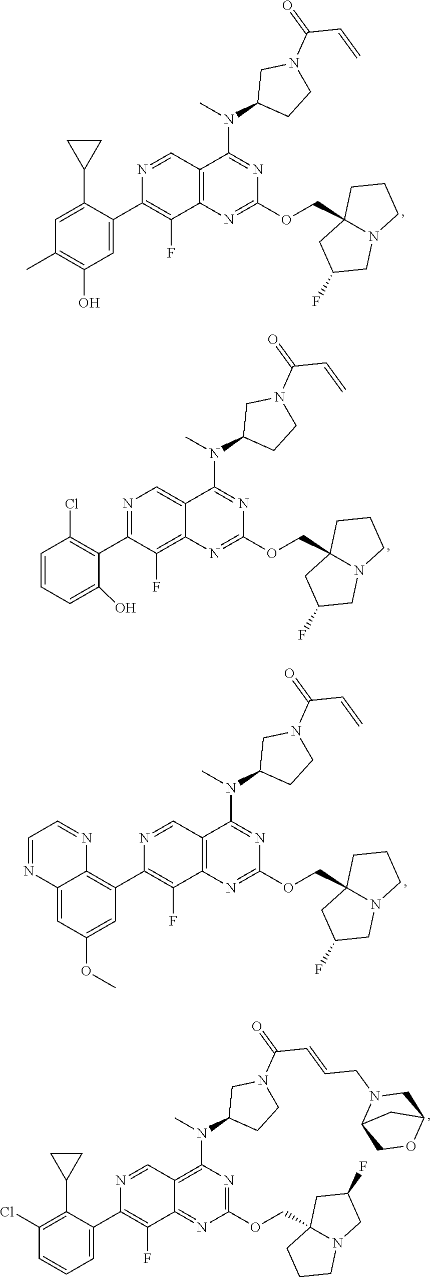

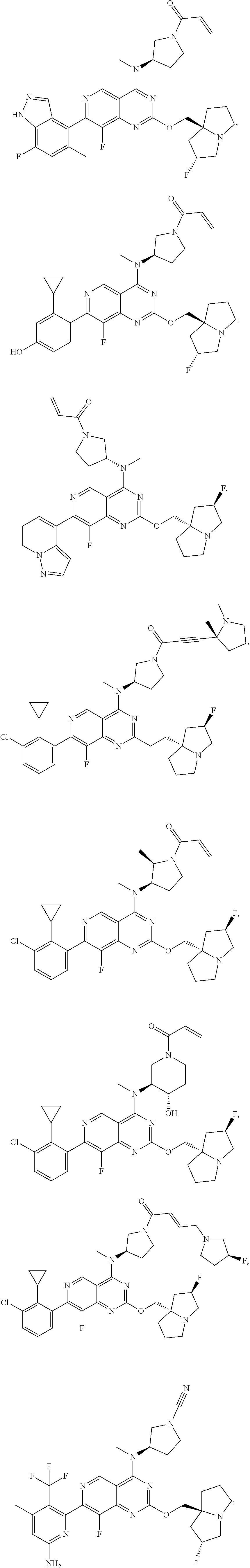

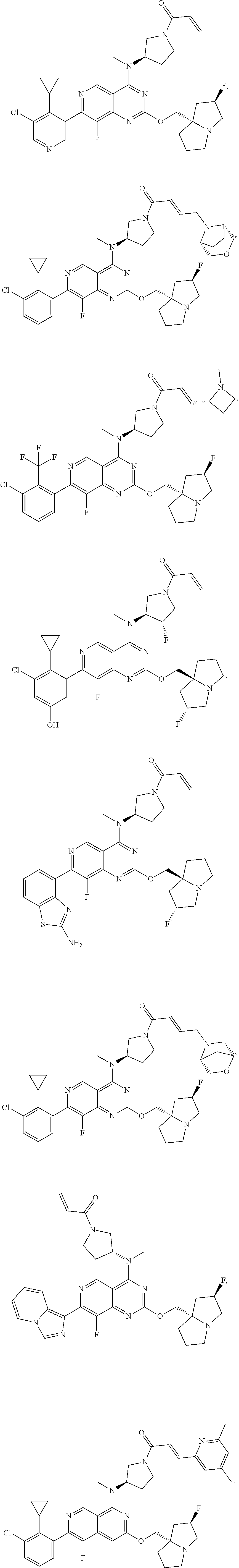

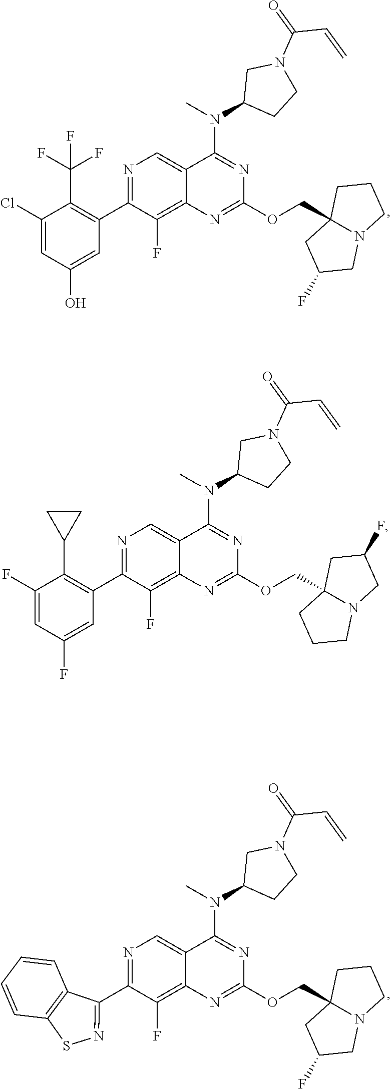 [SMILES: C=CC(=O)N1CC[C@@H](N(C)c2nc(OC[C@@]34CCCN3C[C@H](F)C4)nc3c(F)c(-c4cc(O)cc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(F)cc(F)c4C4CC4)ncc23)C1 ; C=CC(=O)N1CC[C@@H](N(C)c2nc(OC[C@@]34CCCN3C[C@H](F)C4)nc3c(F)c(-c4nsc5ccccc45)ncc23)C1]

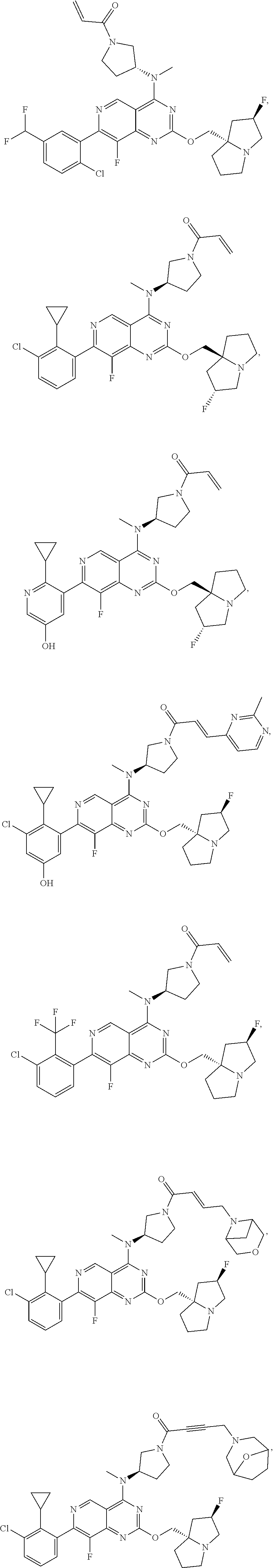

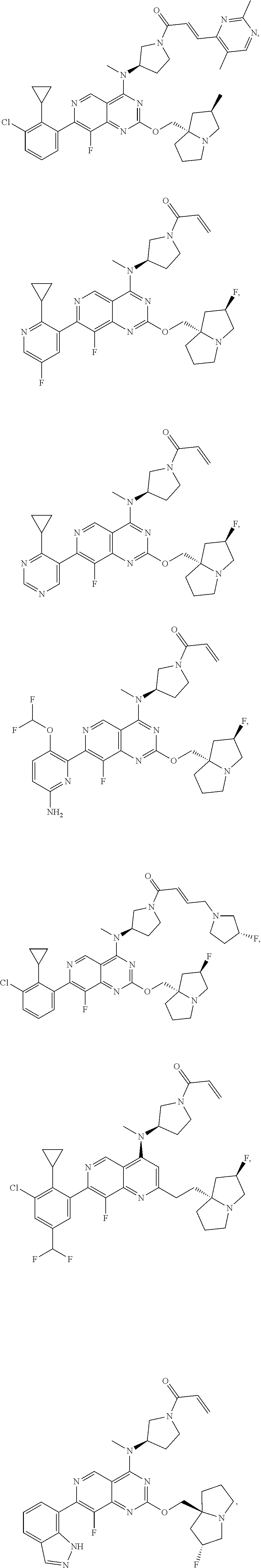

and all salts and isotopologues thereof.

Embodiment 305. The compound of embodiment 1, selected from the group consisting of

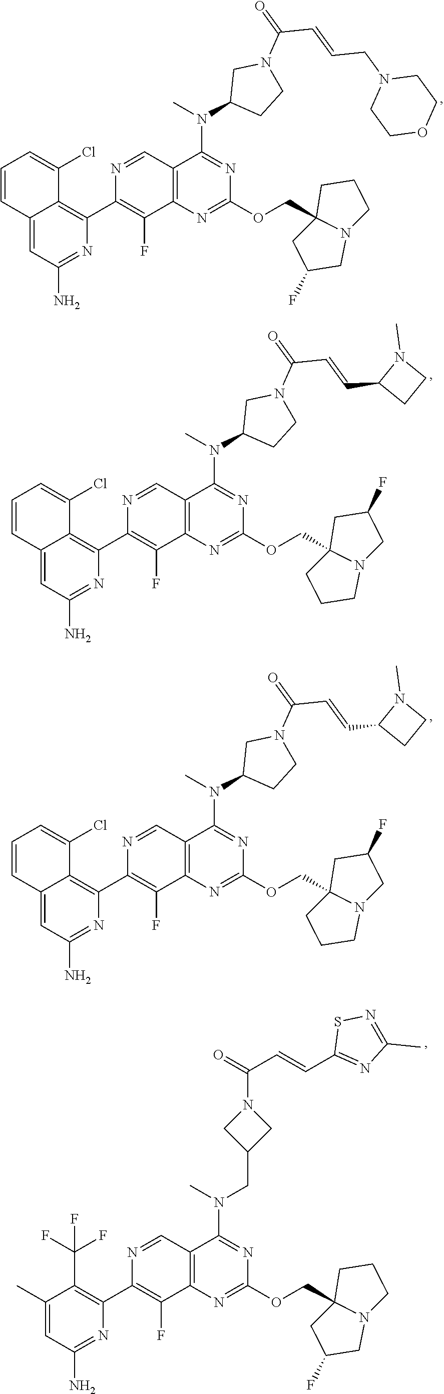

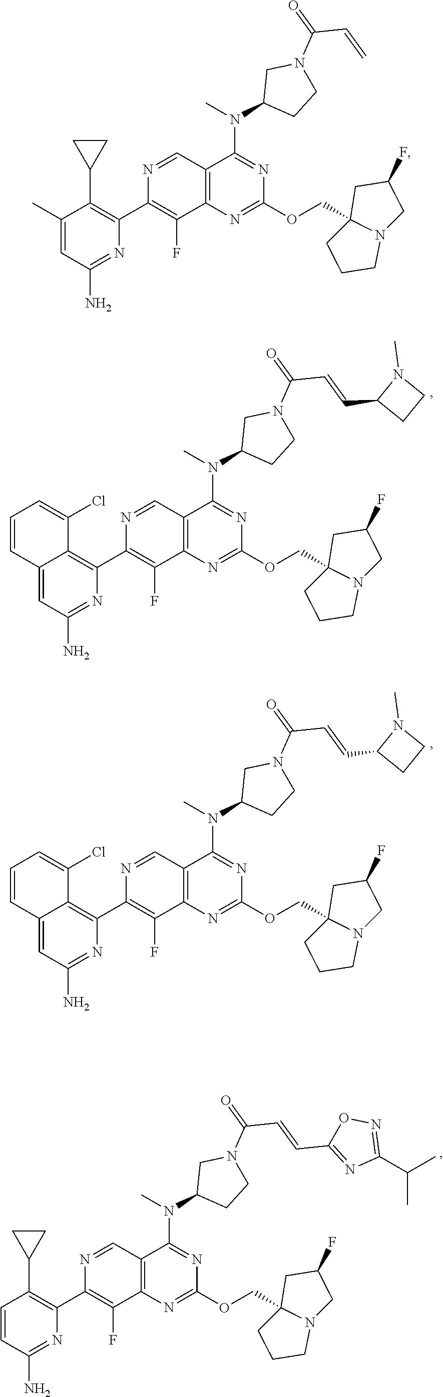 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1 ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1]

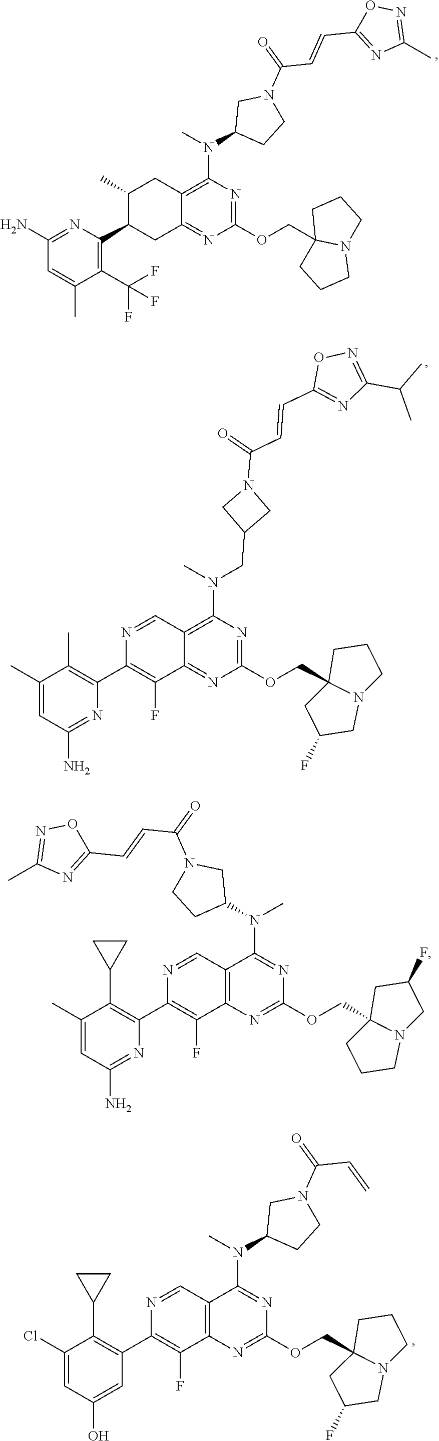

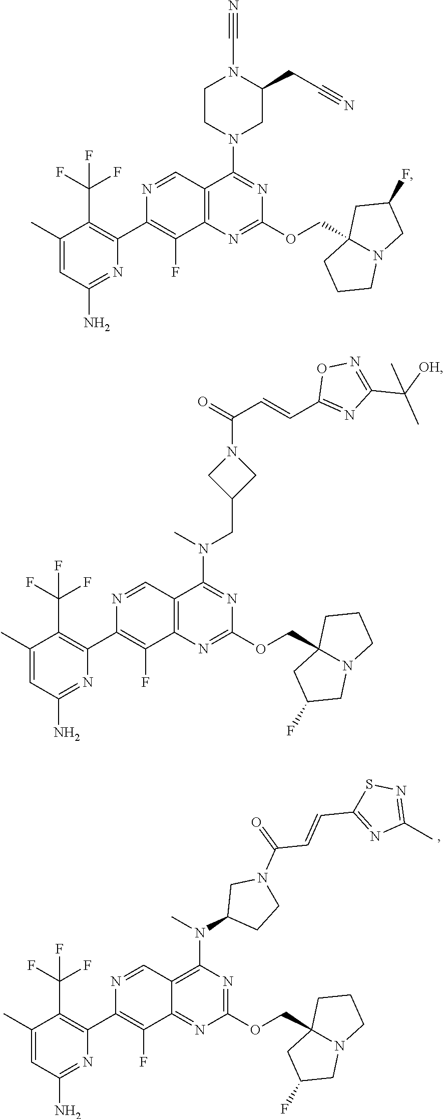

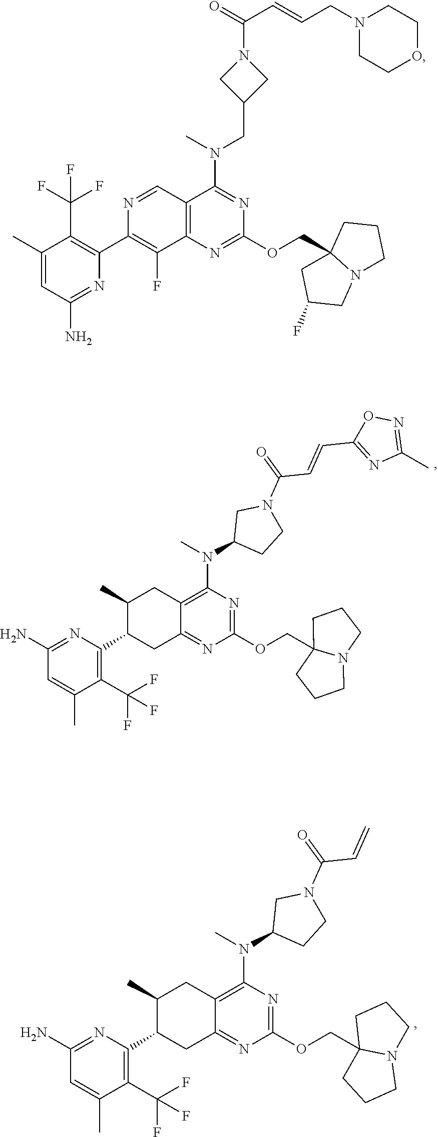 [SMILES: Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F ; Cc1noc(/C=C/C(=O)N2CC[C@@H](N(C)c3nc(OCC45CCCN4CCC5)nc4c3C[C@H](C)[C@@H](c3nc(N)cc(C)c3C(F)(F)F)C4)C2)n1 ; C=CC(=O)N1CC[C@@H](N(C)c2nc(OCC34CCCN3CCC4)nc3c2C[C@H](C)[C@@H](c2nc(N)cc(C)c2C(F)(F)F)C3)C1]

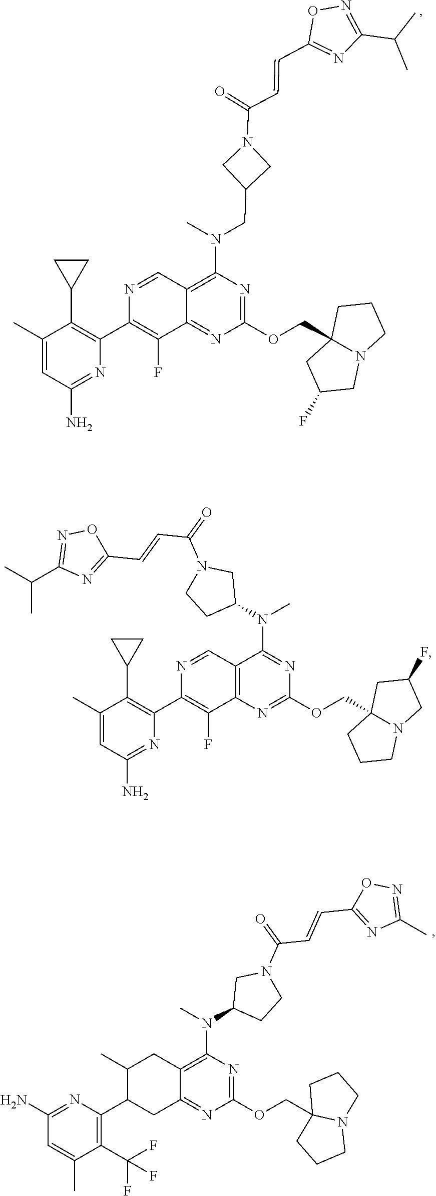

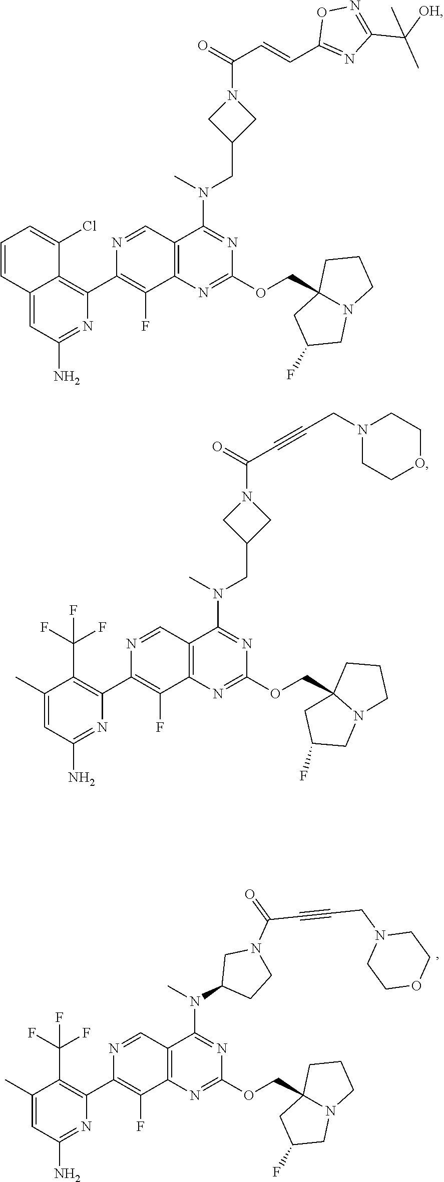 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12 ; Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F]

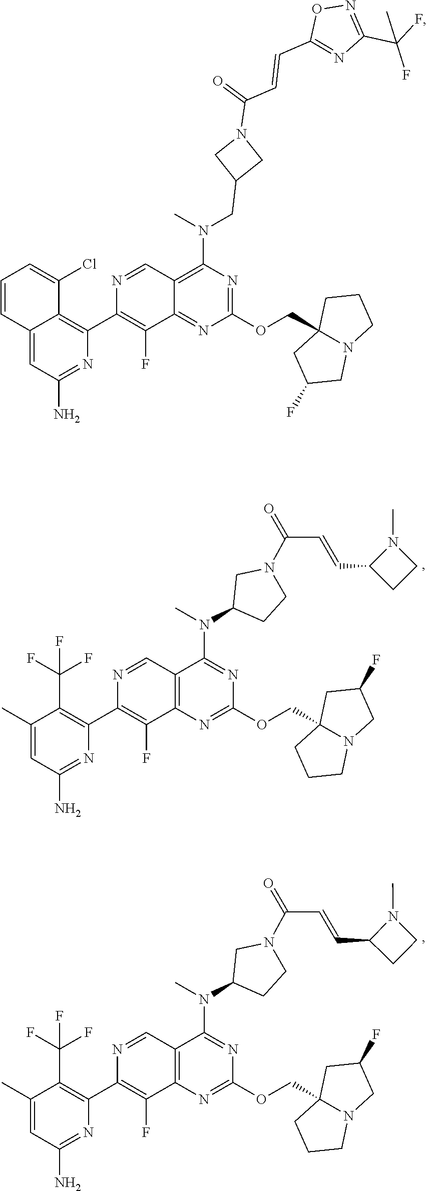 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12 ; Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F]

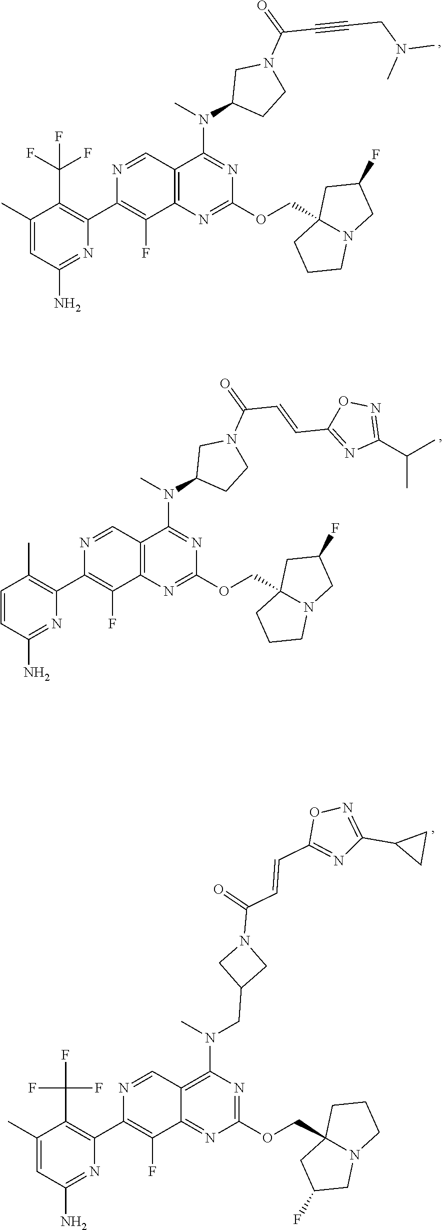

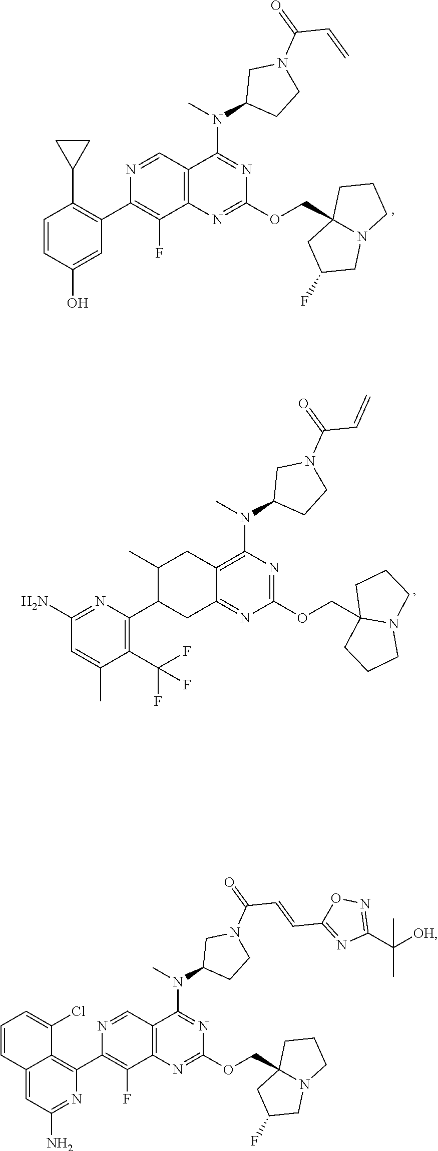 [SMILES: C=CC(=O)N1CC[C@@H](N(C)c2nc(OC[C@@]34CCCN3C[C@H](F)C4)nc3c(F)c(-c4cc(O)ccc4C4CC4)ncc23)C1 ; C=CC(=O)N1CC[C@@H](N(C)c2nc(OCC34CCCN3CCC4)nc3c2CC(C)C(c2nc(N)cc(C)c2C(F)(F)F)C3)C1 ; CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1]

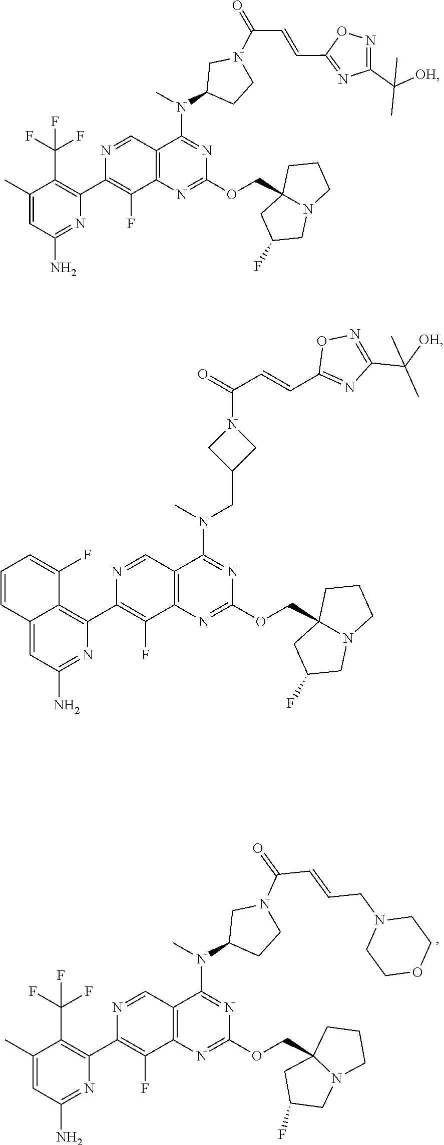 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12 ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F]

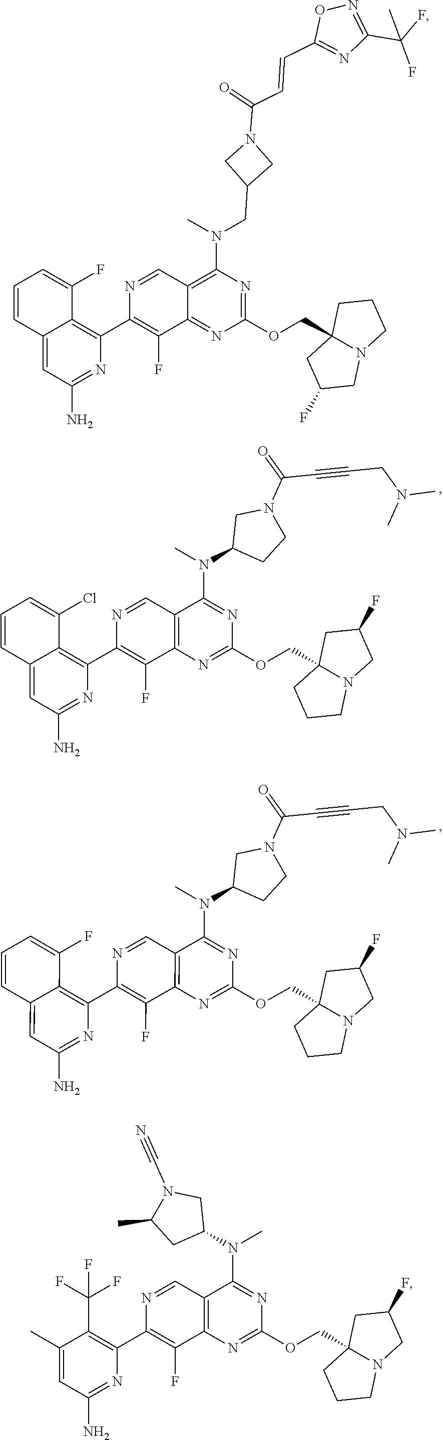

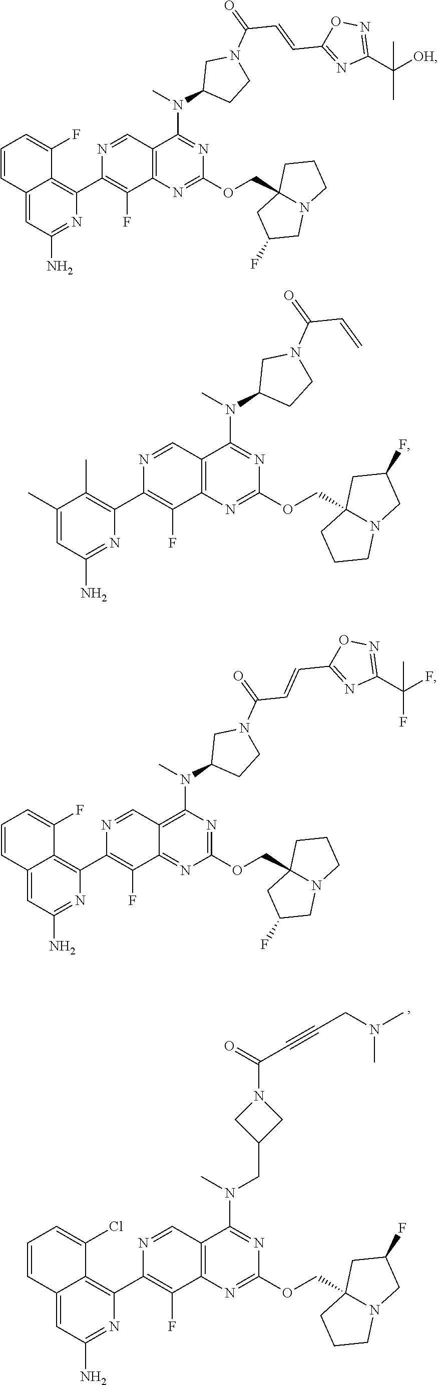 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1 ; CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1]

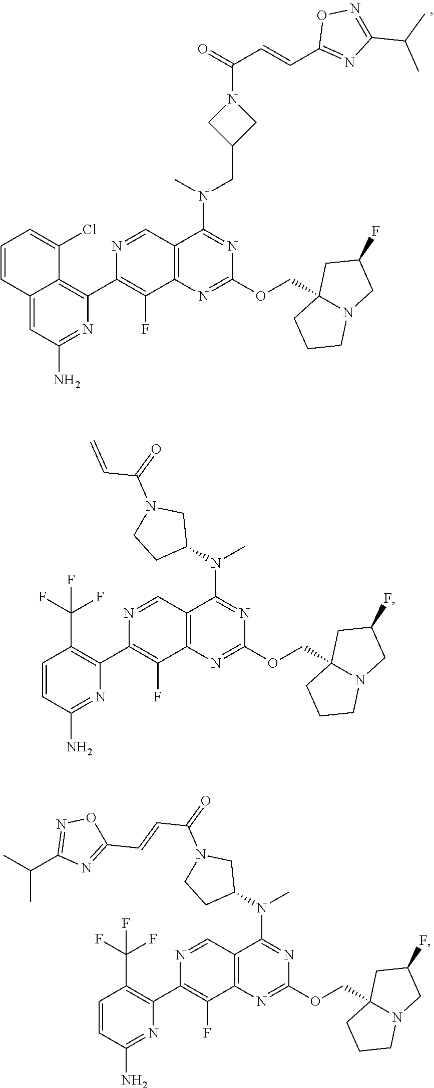

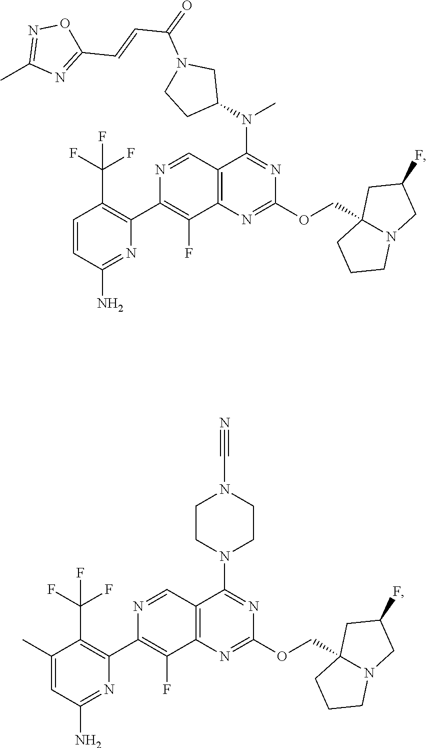 [SMILES: Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1cc(N)nc(-c2ncc3c(N4CCN(C#N)CC4)nc(OC[C@@]45CCCN4C[C@H](F)C5)nc3c2F)c1C(F)(F)F]

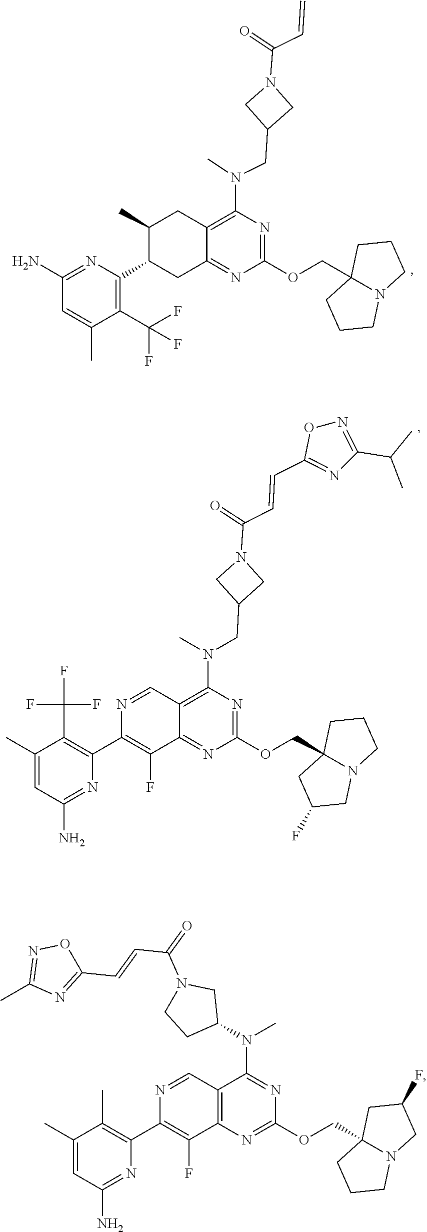

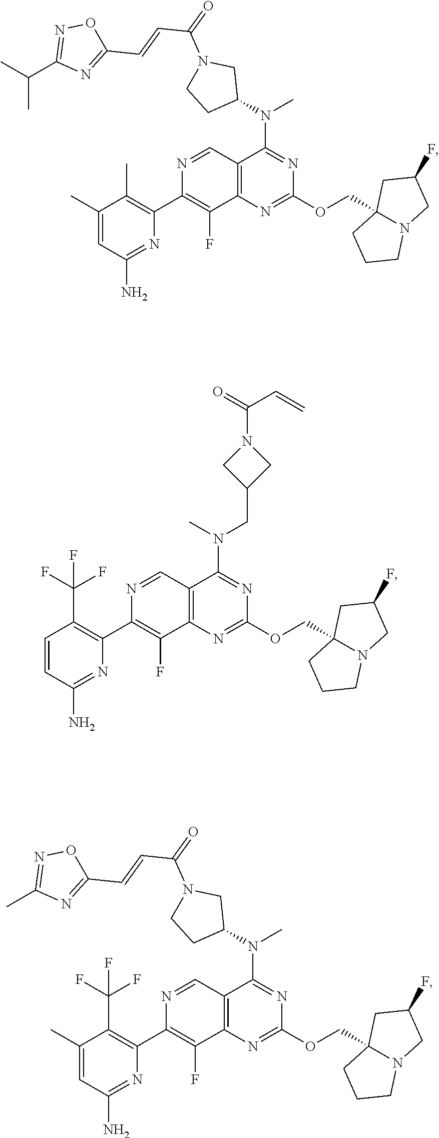 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C ; C=CC(=O)N1CC(CN(C)c2nc(OC[C@@]34CCCN3C[C@H](F)C4)nc3c(F)c(-c4nc(N)ccc4C(F)(F)F)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

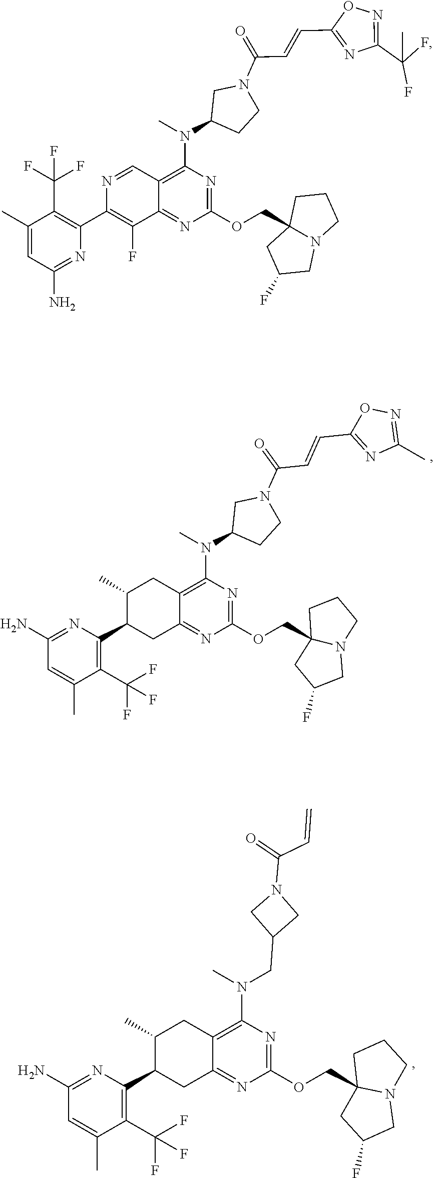 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1 ; C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1]

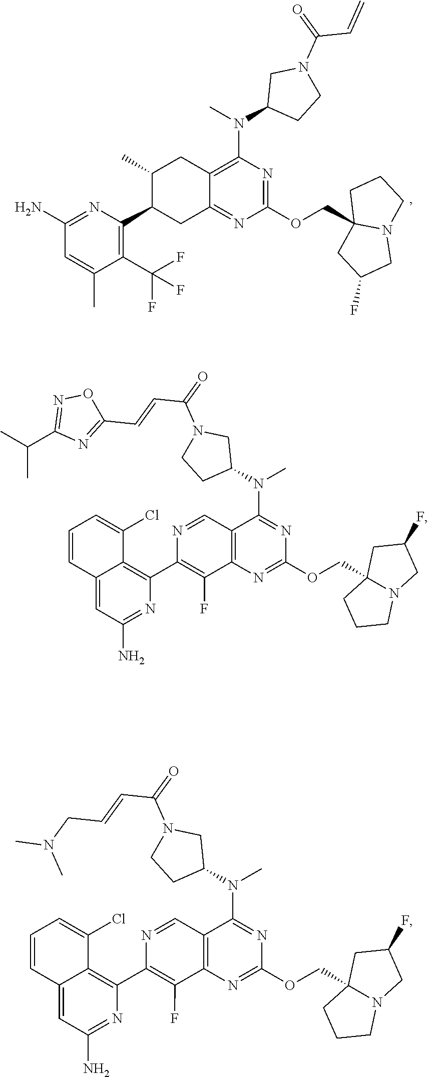 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1 ; CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1]

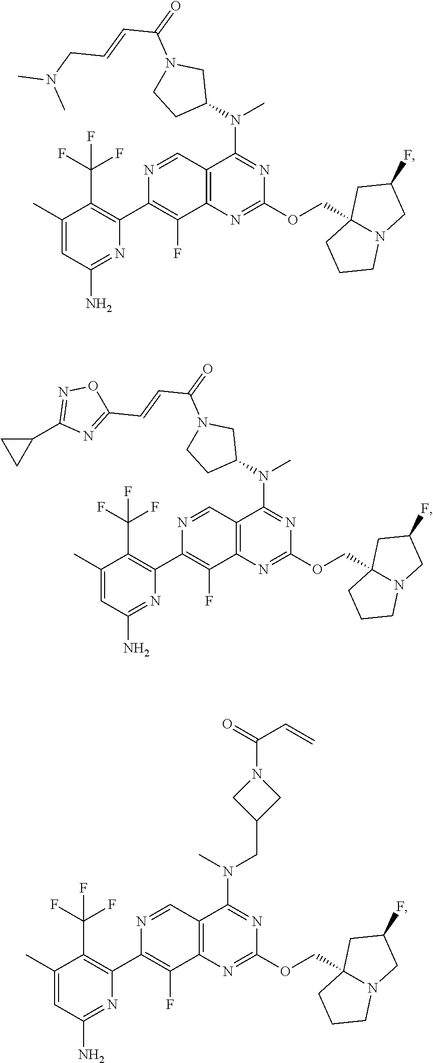 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F ; C=CC(=O)N1CC(CN(C)c2nc(OC[C@@]34CCCN3C[C@H](F)C4)nc3c(F)c(-c4nc(N)cc(C)c4C(F)(F)F)ncc23)C1]

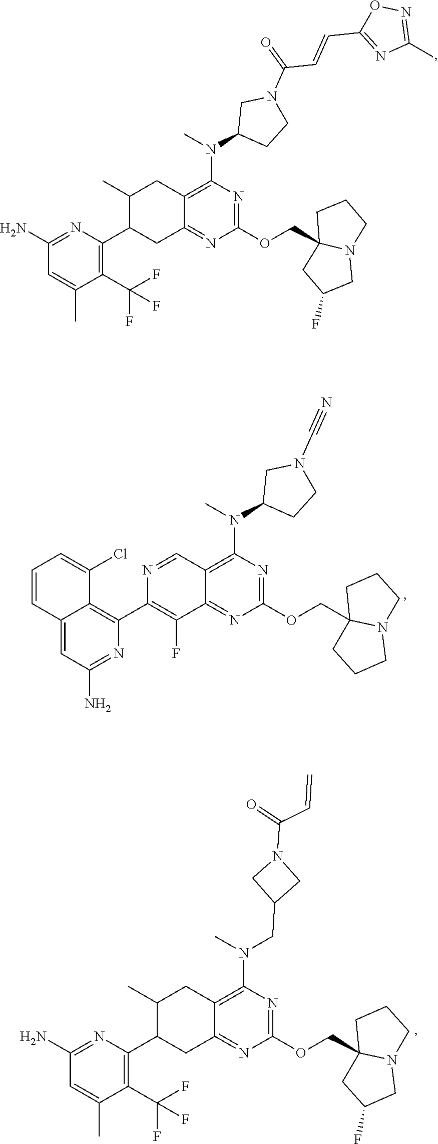

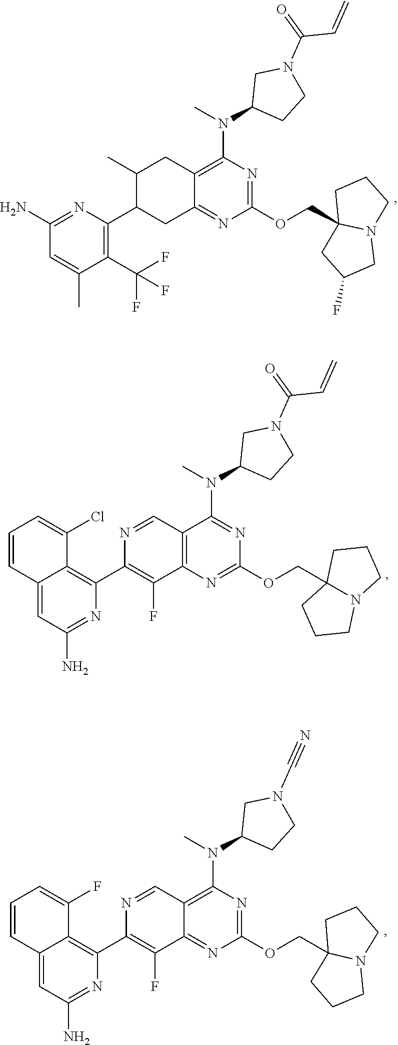 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1 ; C=CC(=O)N1CC[C@@H](N(C)c2nc(OCC34CCCN3CCC4)nc3c(F)c(-c4nc(N)cc5cccc(Cl)c45)ncc23)C1 ; CN(c1nc(OCC23CCCN2CCC3)nc2c(F)c(-c3nc(N)cc4cccc(F)c34)ncc12)[C@@H]1CCN(C#N)C1]

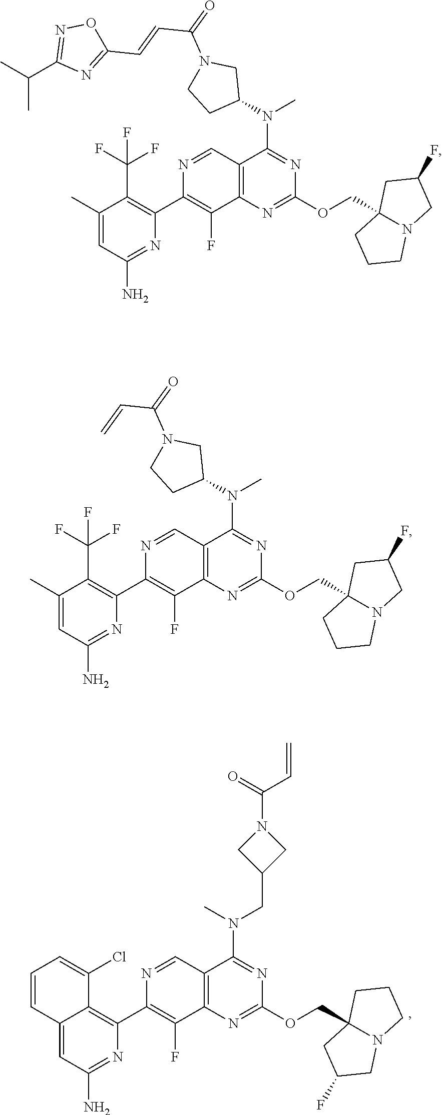 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F ; C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1 ; C=CC(=O)N1CC(CN(C)c2nc(OC[C@@]34CCCN3C[C@H](F)C4)nc3c(F)c(-c4nc(N)cc5cccc(Cl)c45)ncc23)C1]

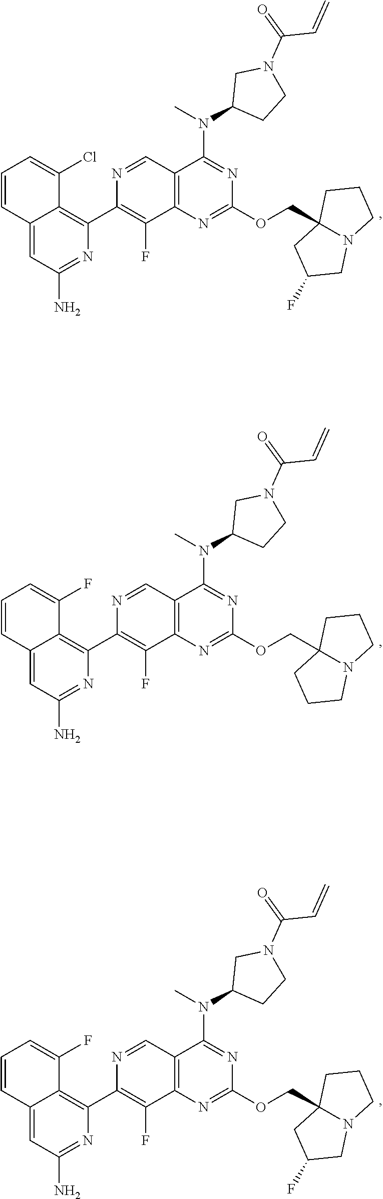 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OCC34CCCN3CCC4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4nc(N)cc5cccc(F)c45)ncc23)C1]

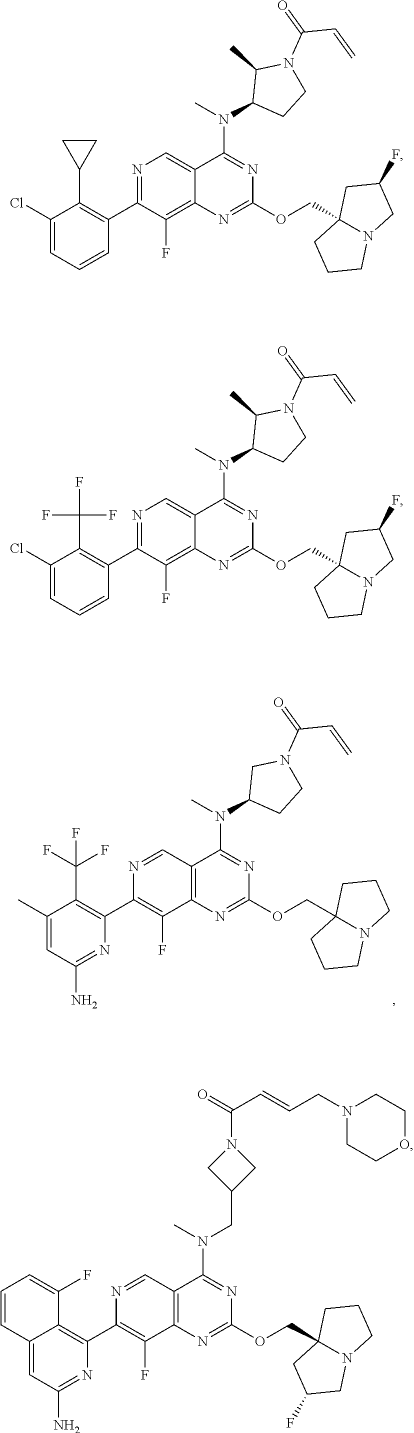 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OCC34CCCN3CCC4)nc3c(F)c(-c4nc(N)cc(C)c4C(F)(F)F)ncc23)C1 ; CN(CC1CN(C(=O)/C=C/CN2CCOCC2)C1)c1nc(OC[C@@]23CCCN2C[C@H](F)C3)nc2c(F)c(-c3nc(N)cc4cccc(F)c34)ncc12]

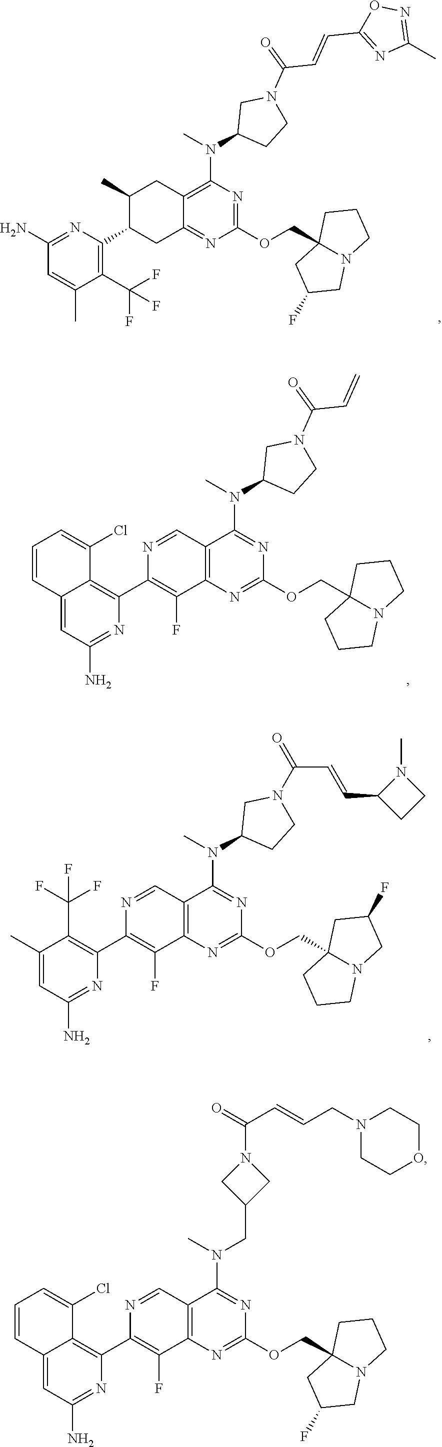

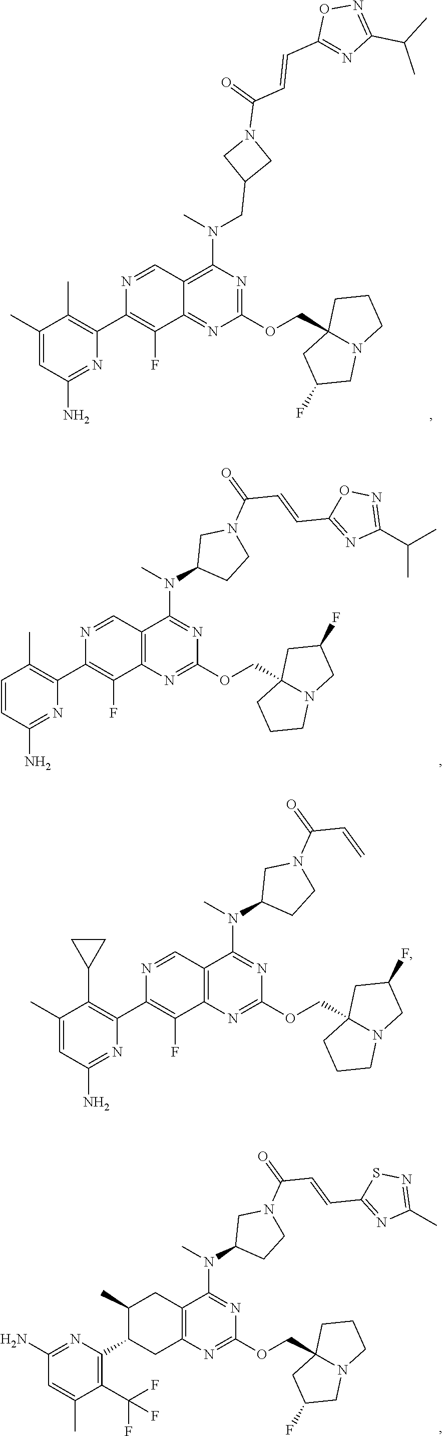

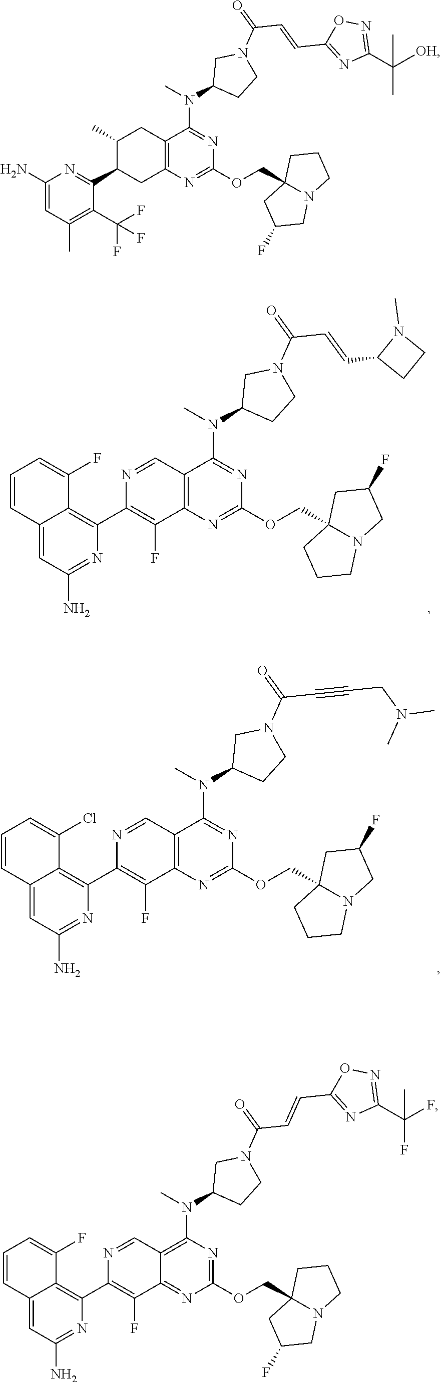 [SMILES: Cc1cc(N)nc([C@@H]2Cc3nc(OC[C@@]45CCCN4C[C@H](F)C5)nc(N(C)[C@@H]4CCN(C(=O)/C=C/c5nc(C(C)(C)O)no5)C4)c3C[C@H]2C)c1C(F)(F)F ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; CN(C)CC#CC(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1]

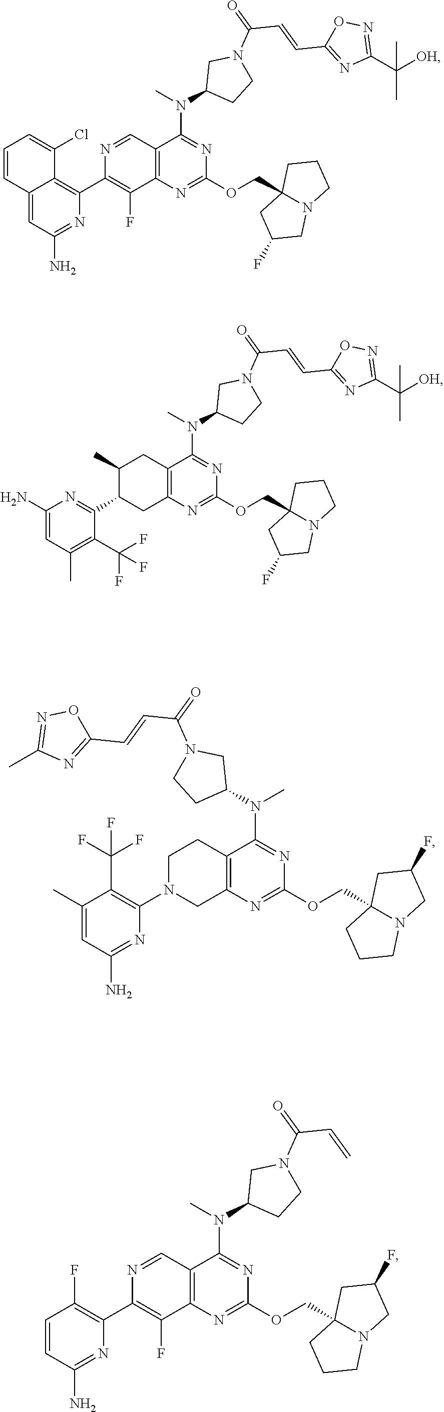 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc([C@H]2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F ; Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1 ; C=CC(=O)N1CC[C@@H](N(C)c2nc(OC[C@@]34CCCN3C[C@H](F)C4)nc3c(F)c(-c4nc(N)ccc4F)ncc23)C1]

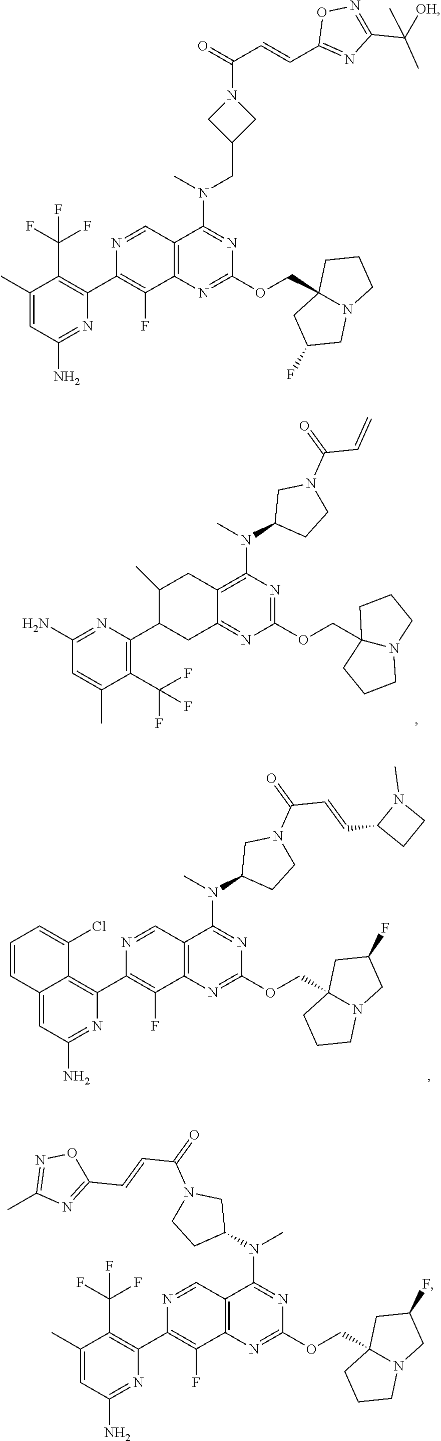 [SMILES: Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F ; C=CC(=O)N1CC[C@@H](N(C)c2nc(OCC34CCCN3CCC4)nc3c2CC(C)C(c2nc(N)cc(C)c2C(F)(F)F)C3)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

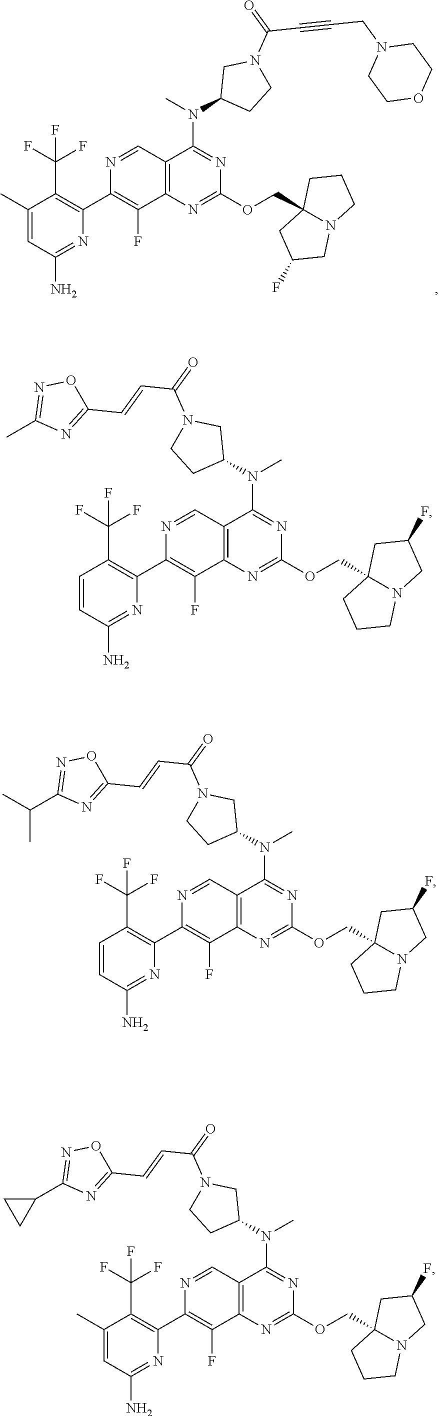 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F]

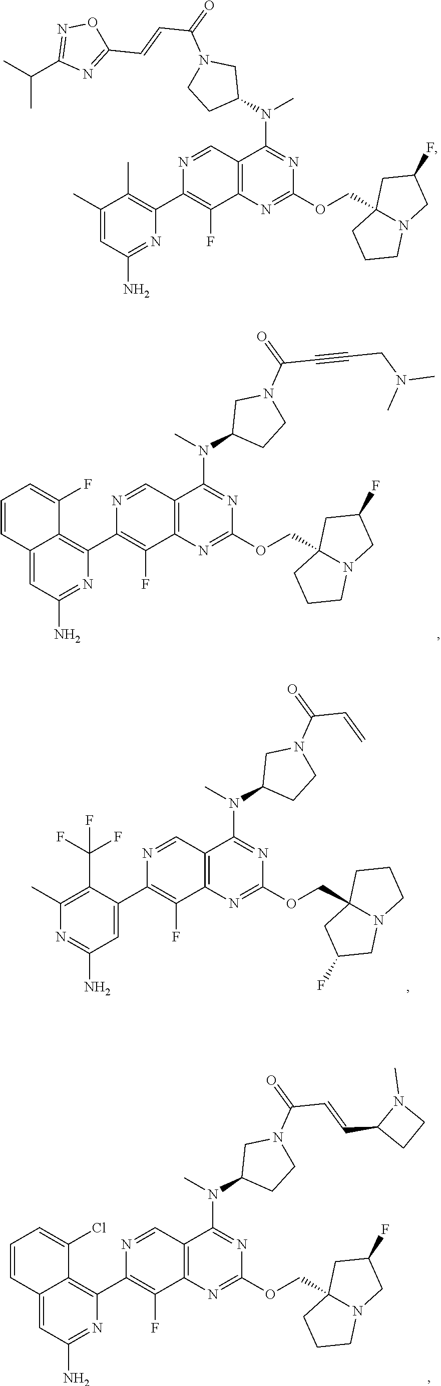

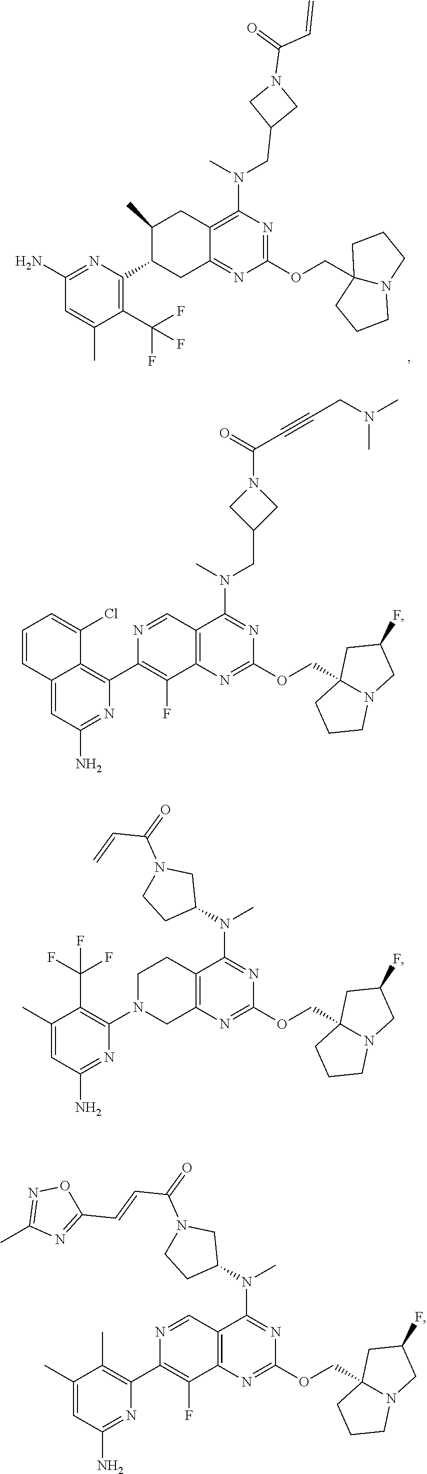

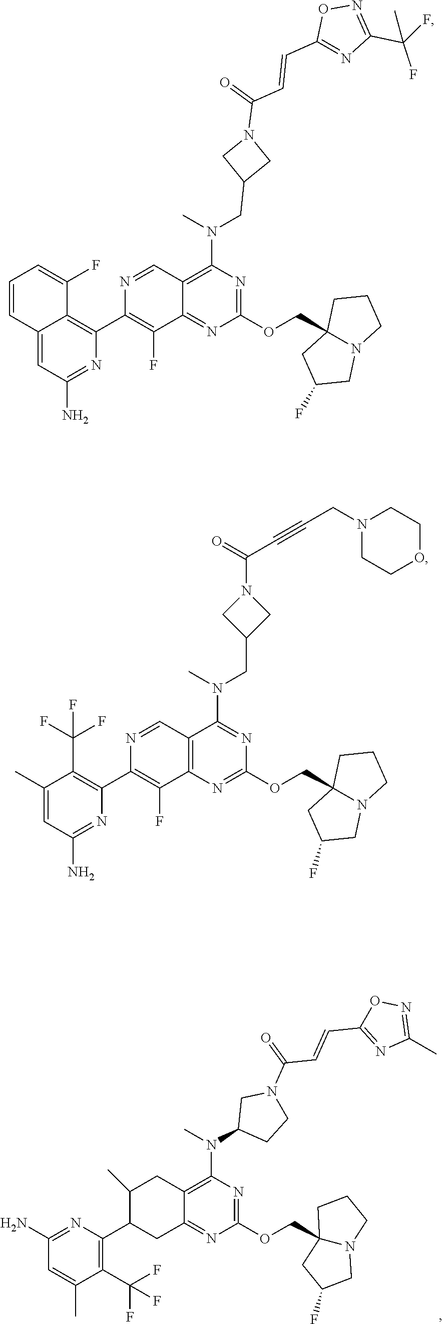 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12 ; Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1]

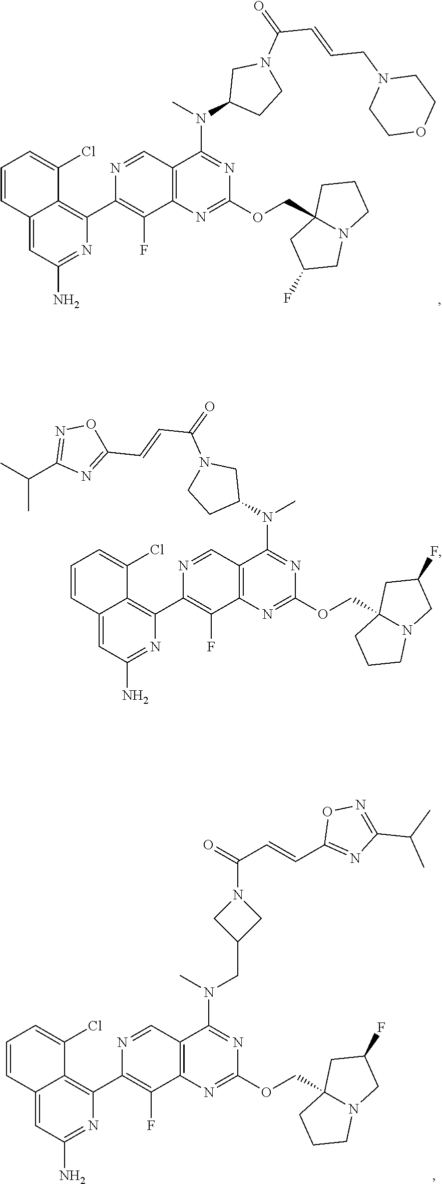 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/CN2CCOCC2)C1 ; CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1]

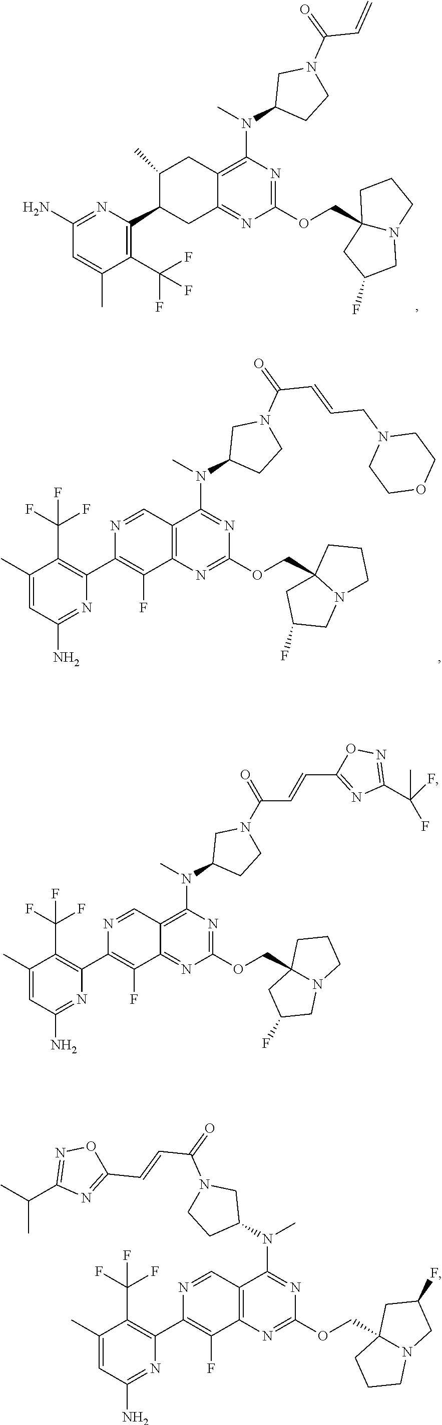 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1 ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F]

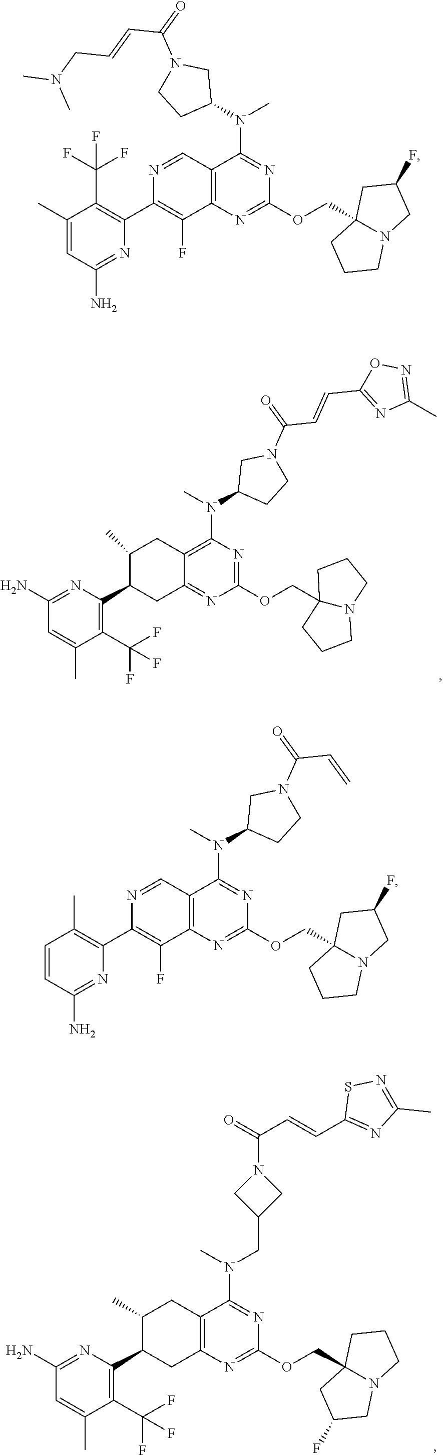 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1 ; C=CC(=O)N1CC[C@@H](N(C)c2nc(OC[C@@]34CCCN3C[C@H](F)C4)nc3c(F)c(-c4nc(N)ccc4C)ncc23)C1 ; Cc1nsc(/C=C/C(=O)N2CC(CN(C)c3nc(OC[C@@]45CCCN4C[C@H](F)C5)nc4c3C[C@@H](C)[C@H](c3nc(N)cc(C)c3C(F)(F)F)C4)C2)n1]

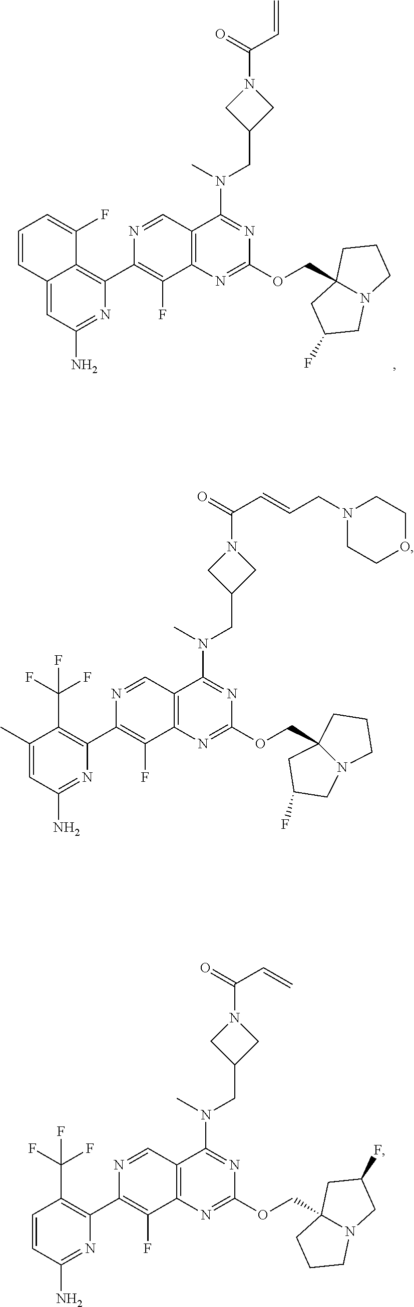 [SMILES: C=CC(=O)N1CC(CN(C)c2nc(OC[C@@]34CCCN3C[C@H](F)C4)nc3c(F)c(-c4nc(N)cc5cccc(F)c45)ncc23)C1 ; Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F ; C=CC(=O)N1CC(CN(C)c2nc(OC[C@@]34CCCN3C[C@H](F)C4)nc3c(F)c(-c4nc(N)ccc4C(F)(F)F)ncc23)C1]

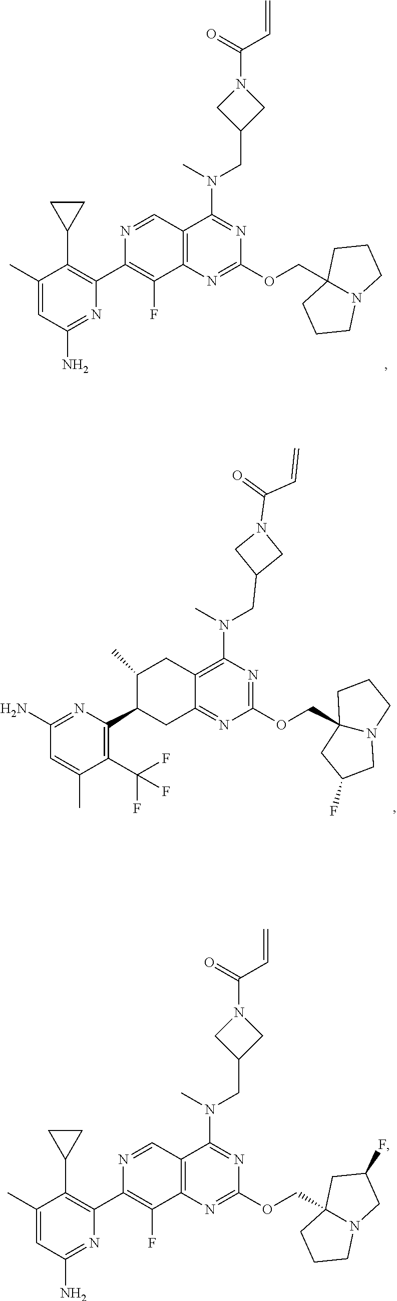 [SMILES: C=CC(=O)N1CC(CN(C)c2nc(OCC34CCCN3CCC4)nc3c(F)c(-c4nc(N)cc(C)c4C4CC4)ncc23)C1 ; C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)cc(C)c4C4CC4)ncc23)C1]

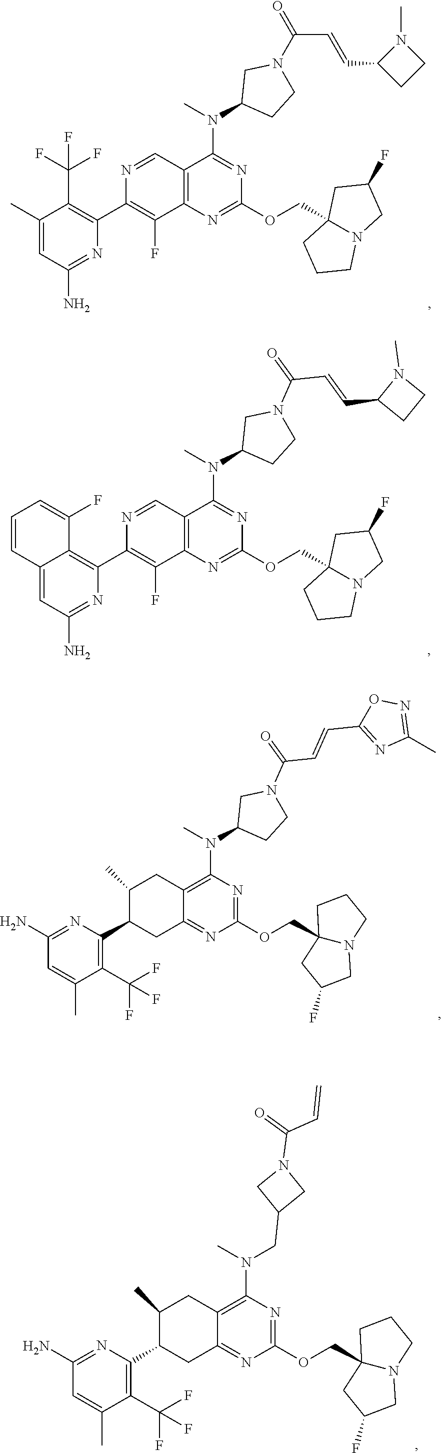 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1 ; C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1]

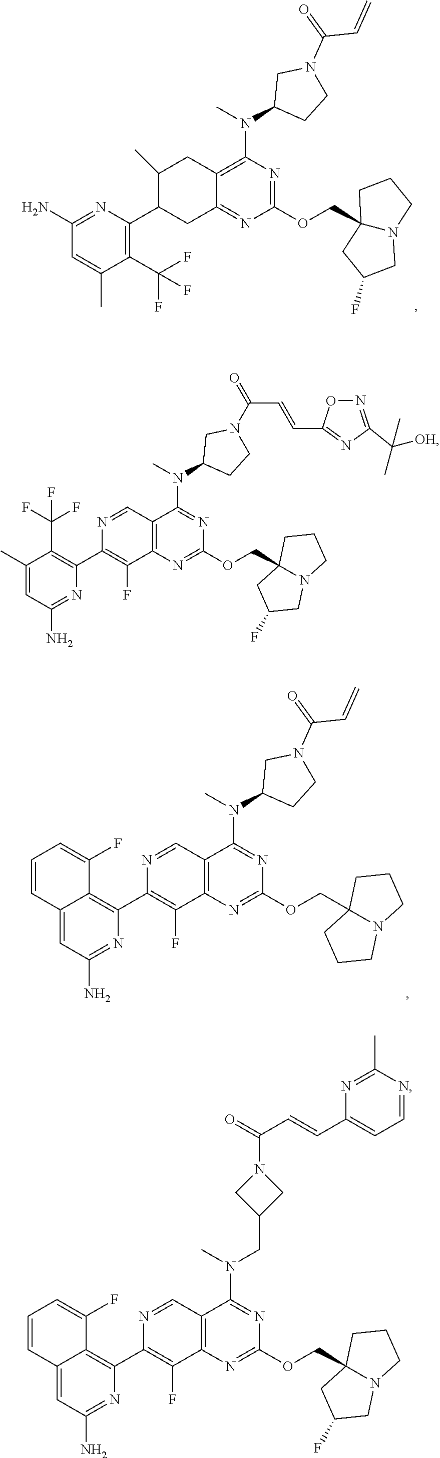 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1 ; Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; C=CC(=O)N1CC[C@@H](N(C)c2nc(OCC34CCCN3CCC4)nc3c(F)c(-c4nc(N)cc5cccc(F)c45)ncc23)C1 ; Cc1nccc(/C=C/C(=O)N2CC(CN(C)c3nc(OC[C@@]45CCCN4C[C@H](F)C5)nc4c(F)c(-c5nc(N)cc6cccc(F)c56)ncc34)C2)n1]

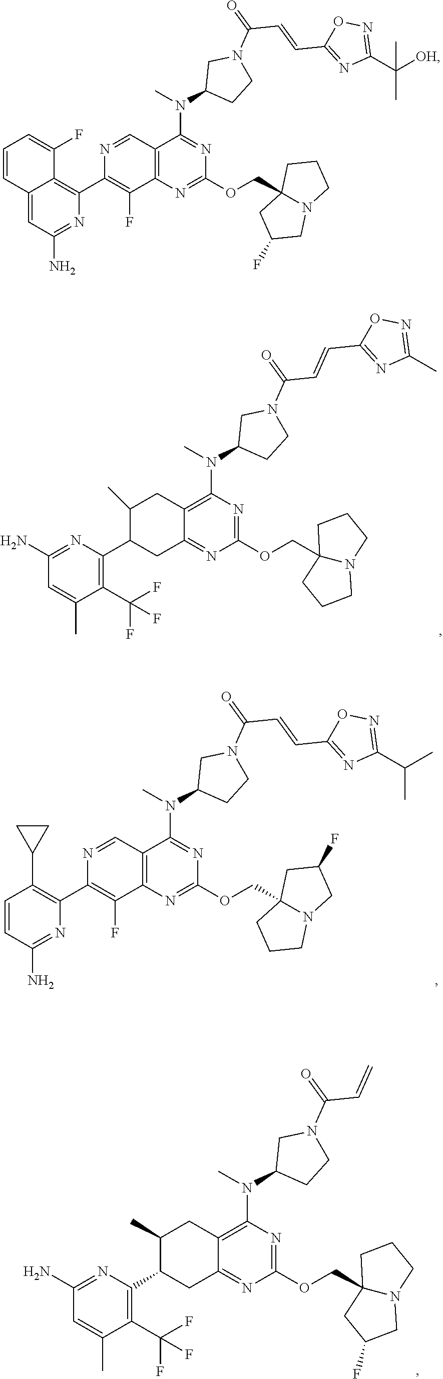 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1 ; C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1]

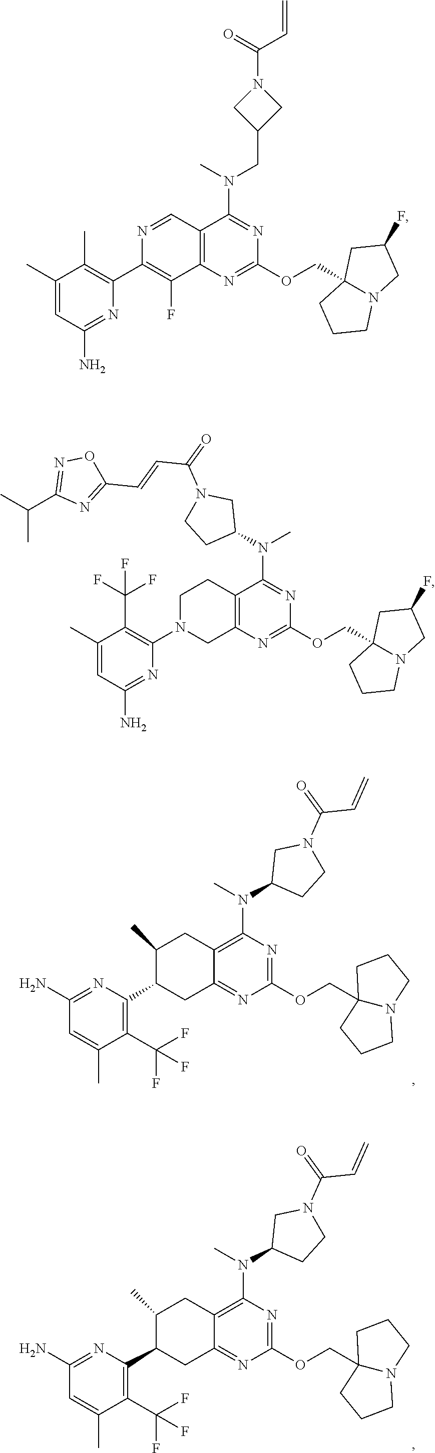 [SMILES: C=CC(=O)N1CC(CN(C)c2nc(OC[C@@]34CCCN3C[C@H](F)C4)nc3c(F)c(-c4nc(N)cc(C)c4C)ncc23)C1 ; Cc1cc(N)nc(N2CCc3c(nc(OC[C@@]45CCCN4C[C@H](F)C5)nc3N(C)[C@@H]3CCN(C(=O)/C=C/c4nc(C(C)C)no4)C3)C2)c1C(F)(F)F ; C=CC(=O)N1CC[C@@H](N(C)c2nc(OCC34CCCN3CCC4)nc3c2C[C@H](C)[C@@H](c2nc(N)cc(C)c2C(F)(F)F)C3)C1 ; C=CC(=O)N1CC[C@@H](N(C)c2nc(OCC34CCCN3CCC4)nc3c2C[C@@H](C)[C@H](c2nc(N)cc(C)c2C(F)(F)F)C3)C1]

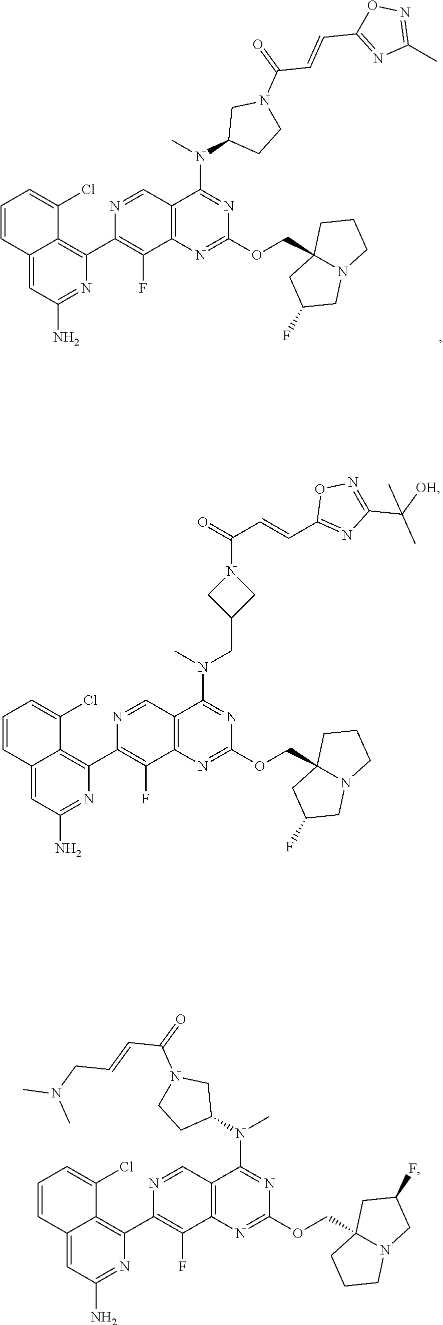

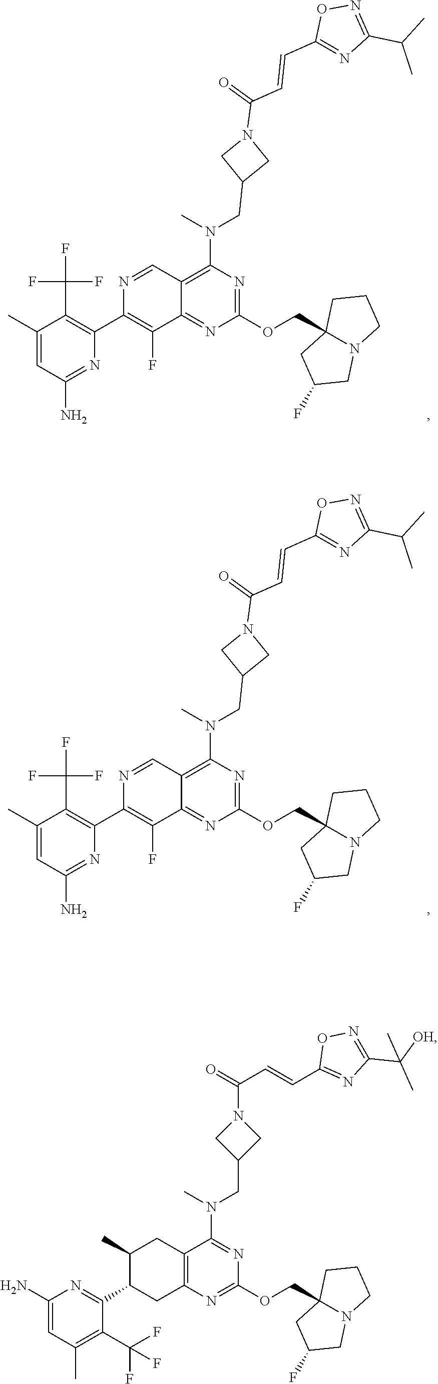

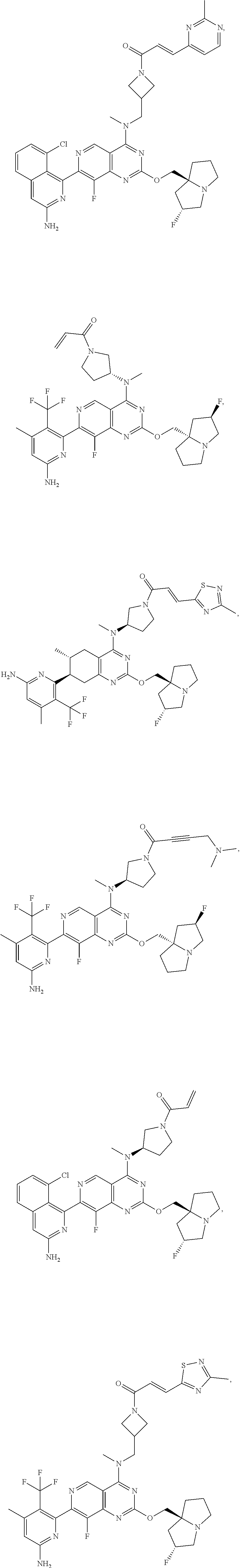

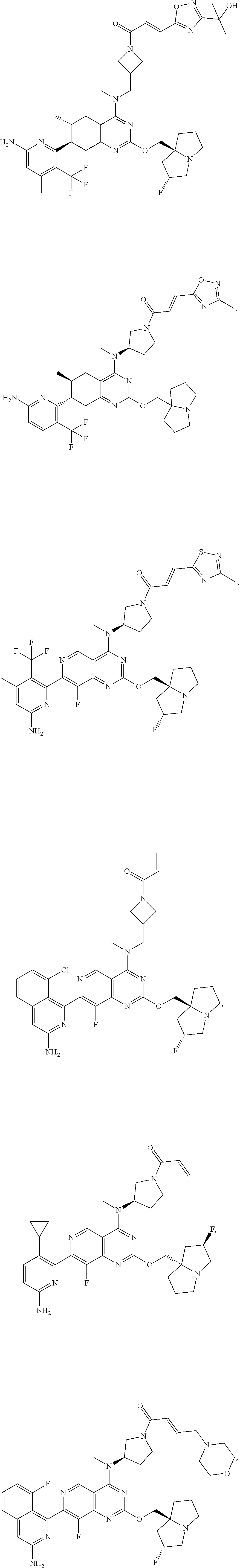

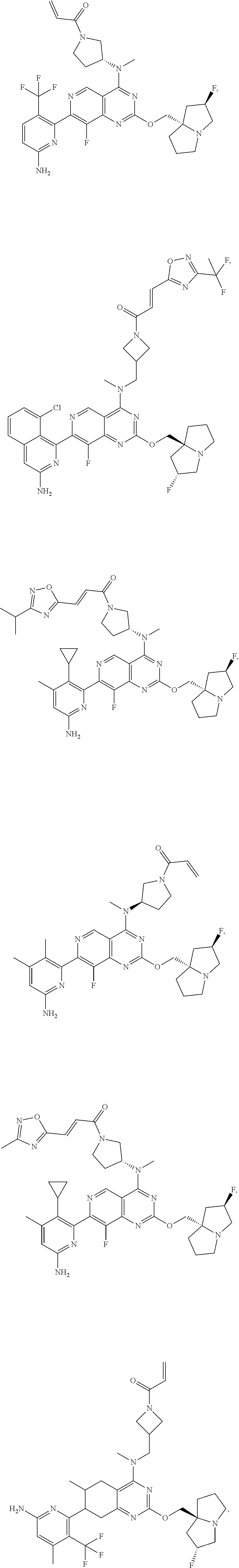

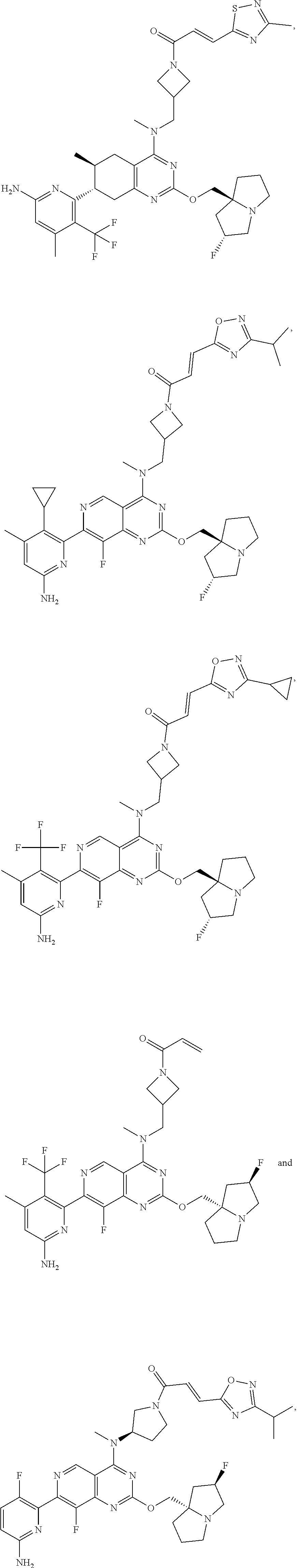

and all salts and isotopologues thereof.

Embodiment 306. The compound of embodiment 1, selected from the group consisting of

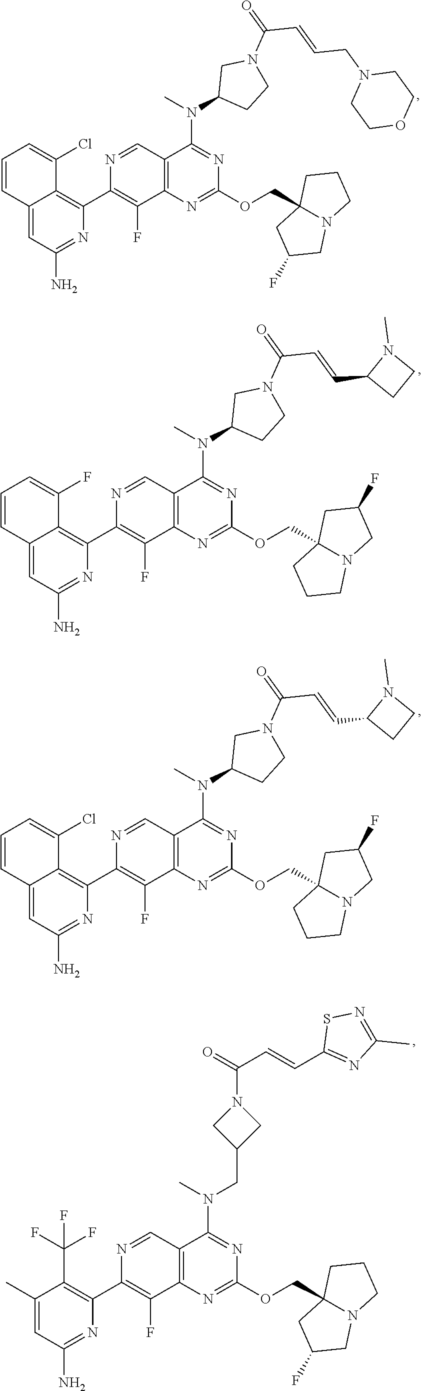

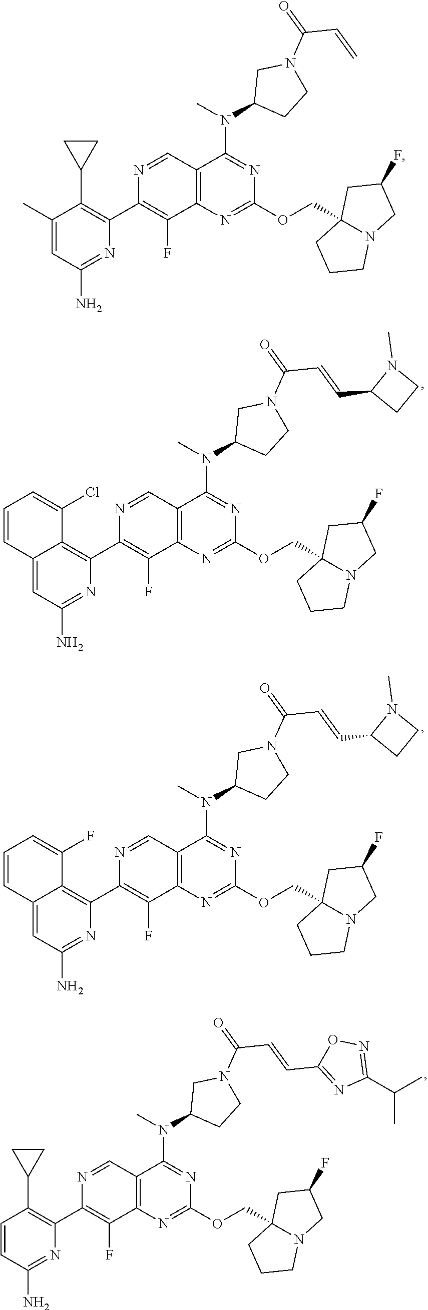 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1 ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1]

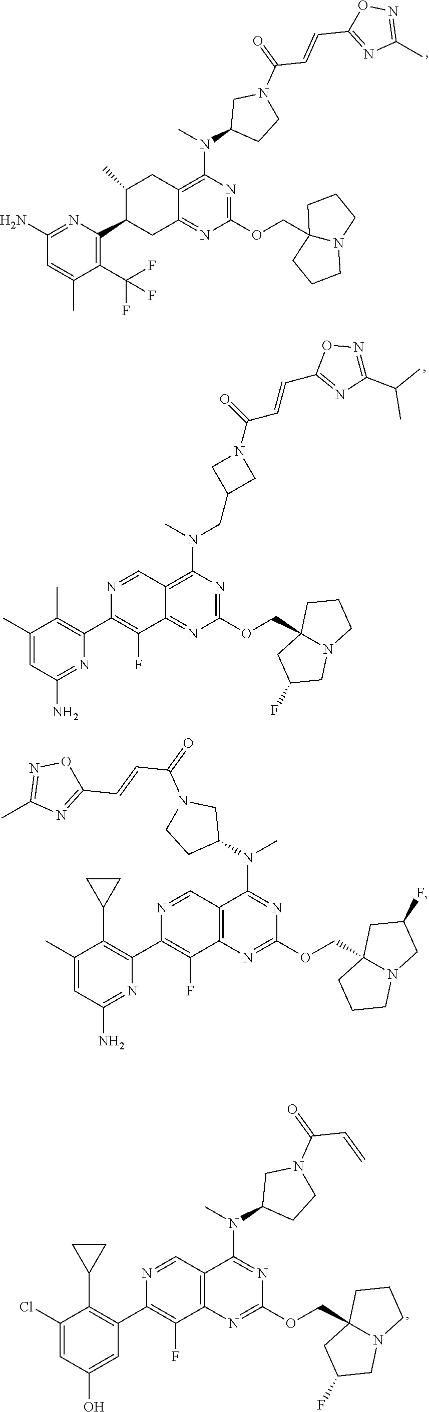

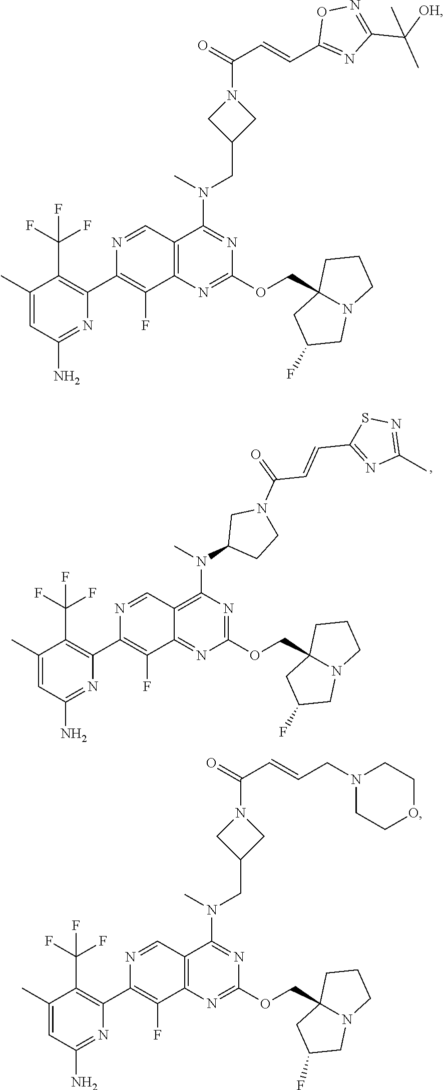

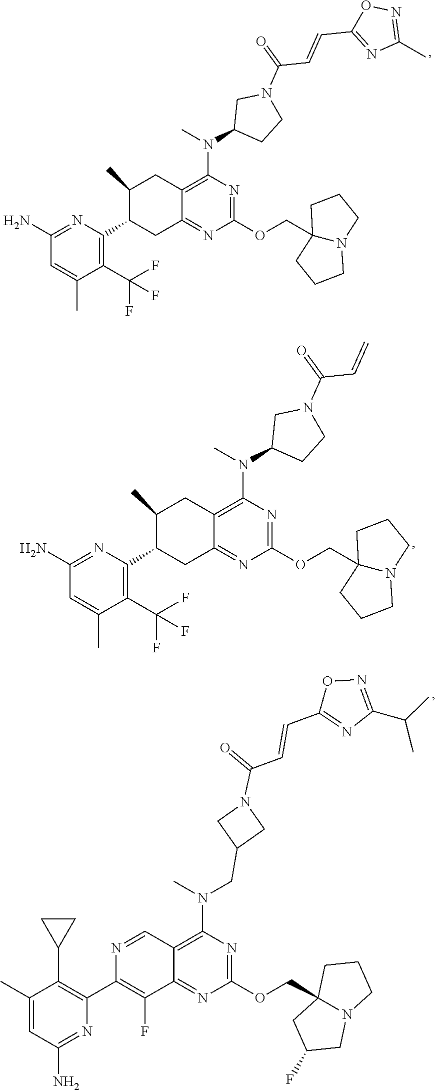

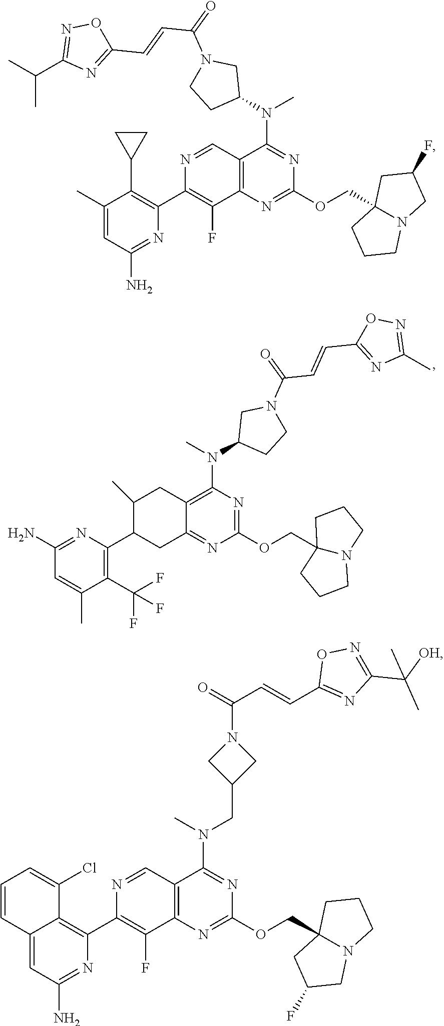 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1 ; Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12]

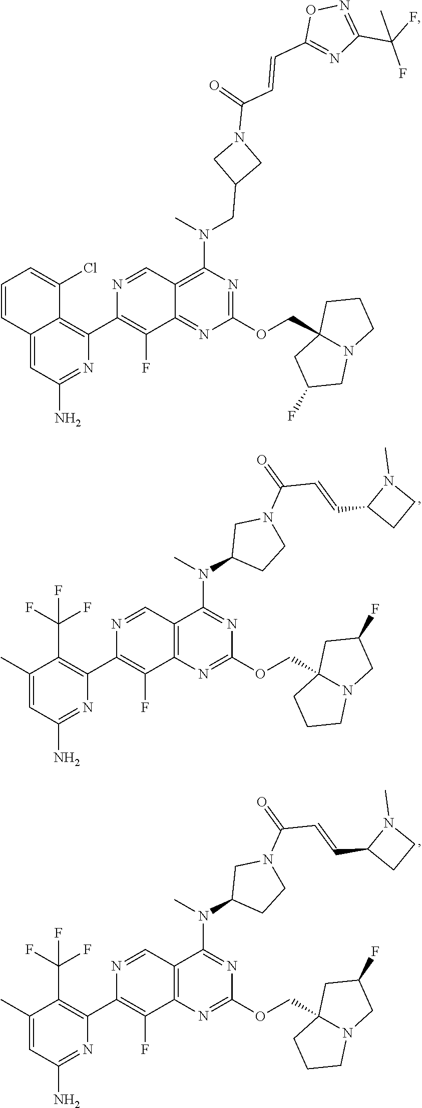 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12 ; Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F]

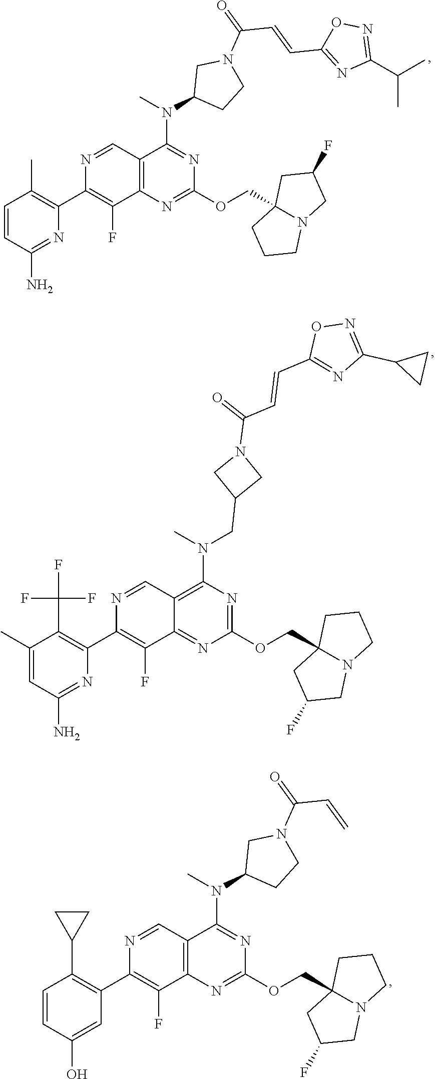

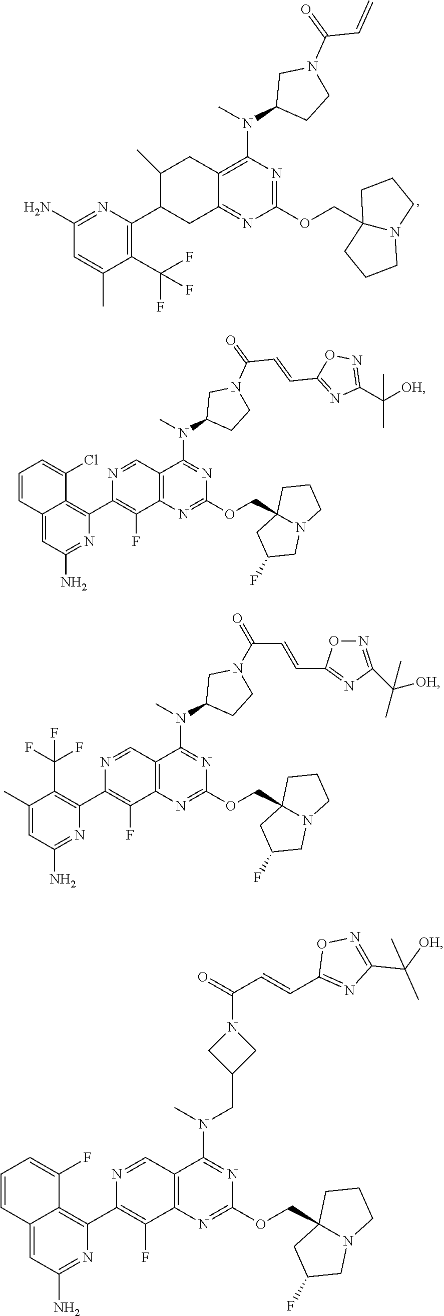 [SMILES: C=CC(=O)N1CC[C@@H](N(C)c2nc(OCC34CCCN3CCC4)nc3c2CC(C)C(c2nc(N)cc(C)c2C(F)(F)F)C3)C1 ; CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12]

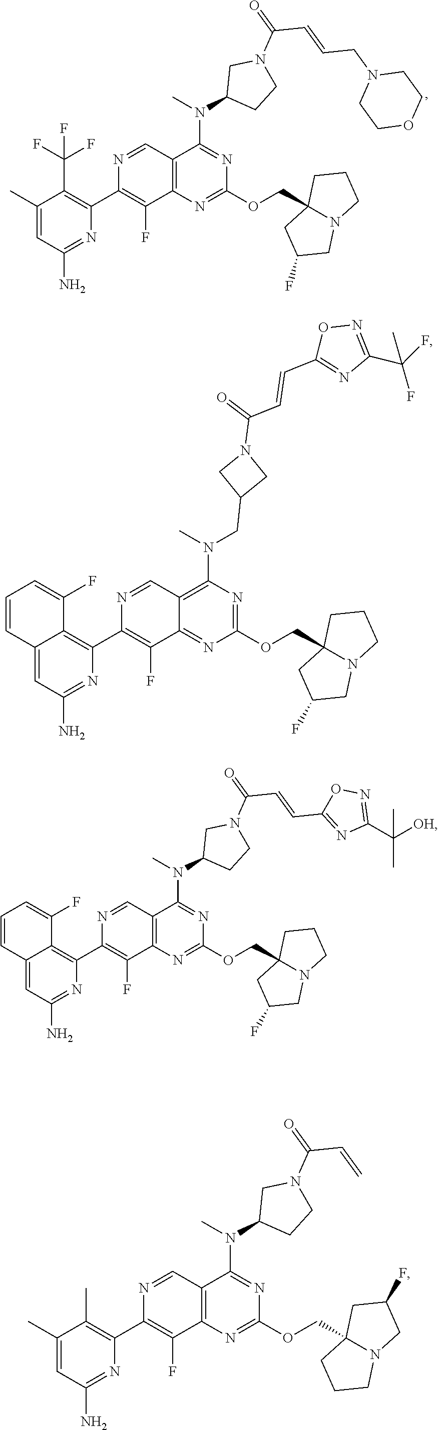 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1]

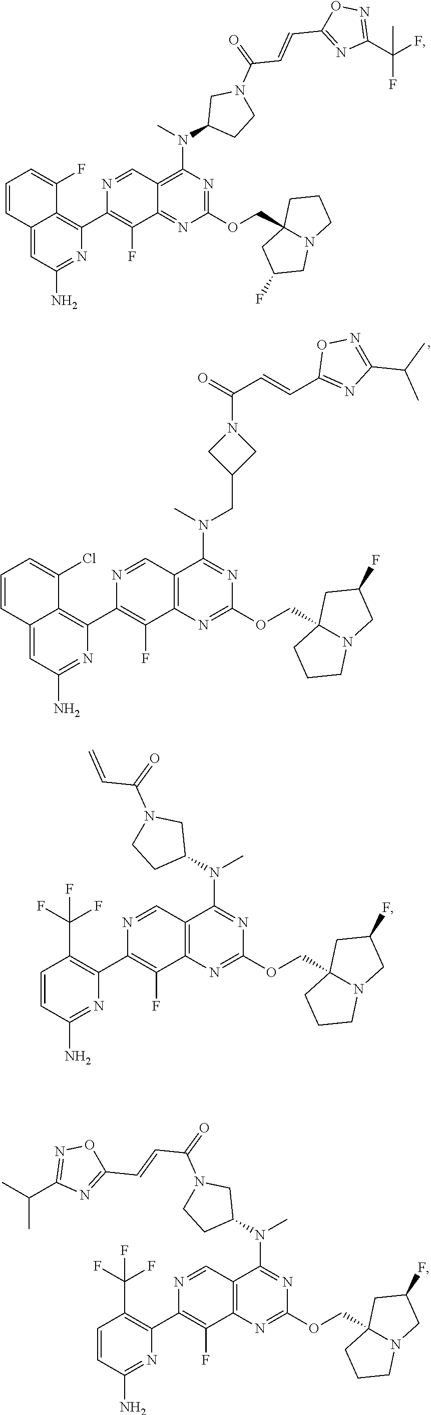

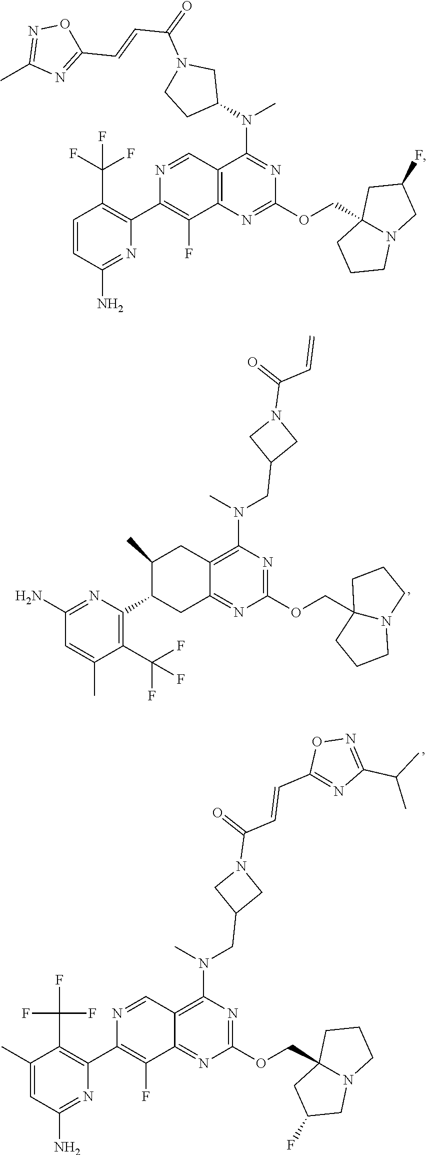 [SMILES: Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; C=CC(=O)N1CC(CN(C)c2nc(OCC34CCCN3CCC4)nc3c2C[C@H](C)[C@@H](c2nc(N)cc(C)c2C(F)(F)F)C3)C1 ; Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C(F)(F)F]

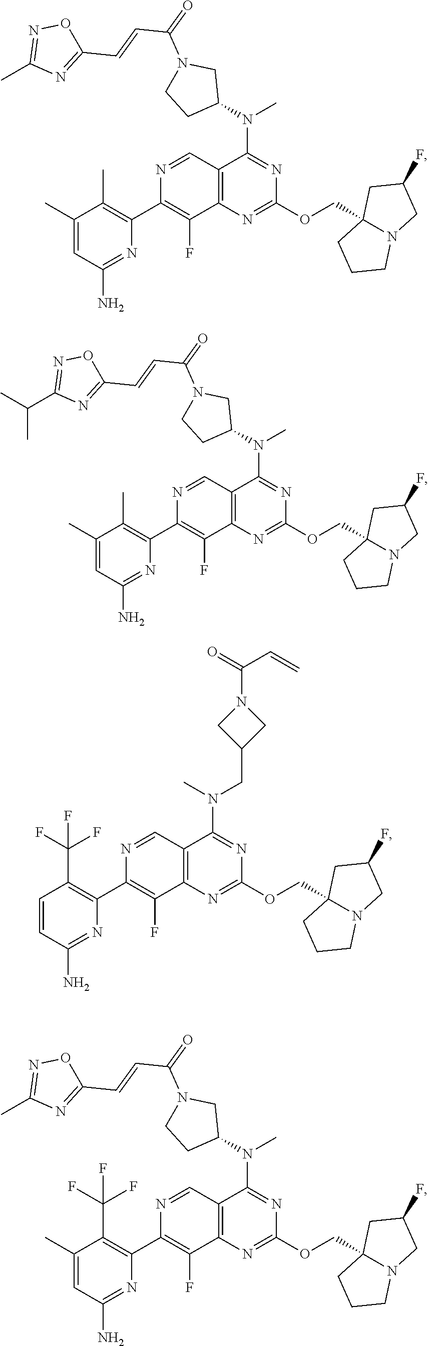 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1 ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C ; C=CC(=O)N1CC(CN(C)c2nc(OC[C@@]34CCCN3C[C@H](F)C4)nc3c(F)c(-c4nc(N)ccc4C(F)(F)F)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

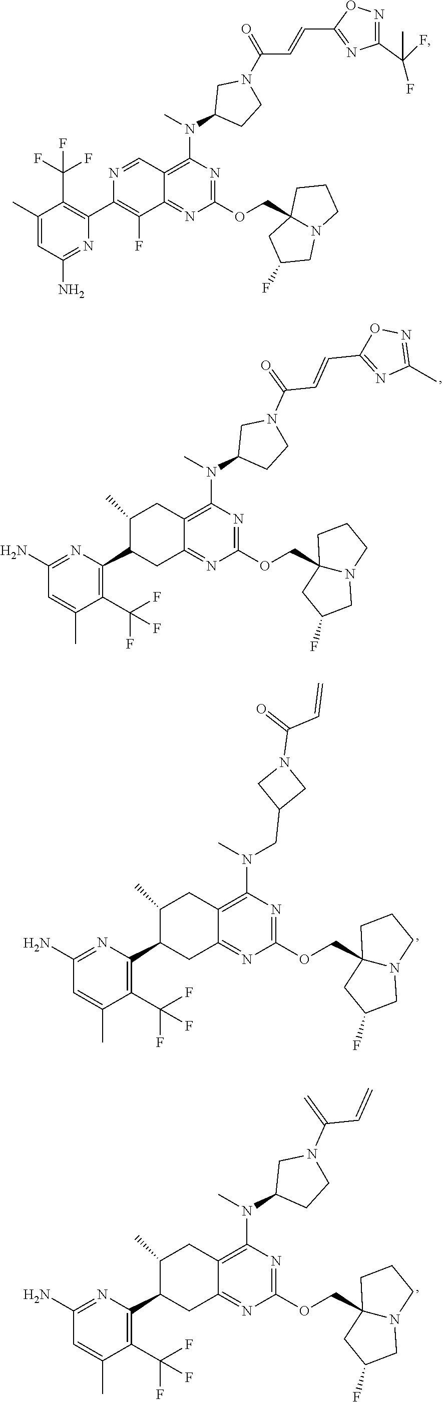

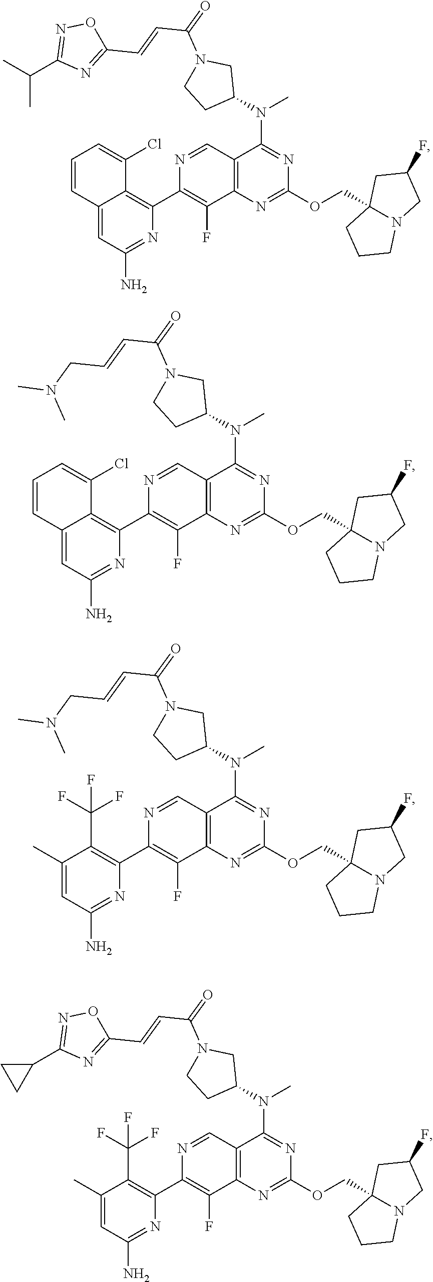 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F]

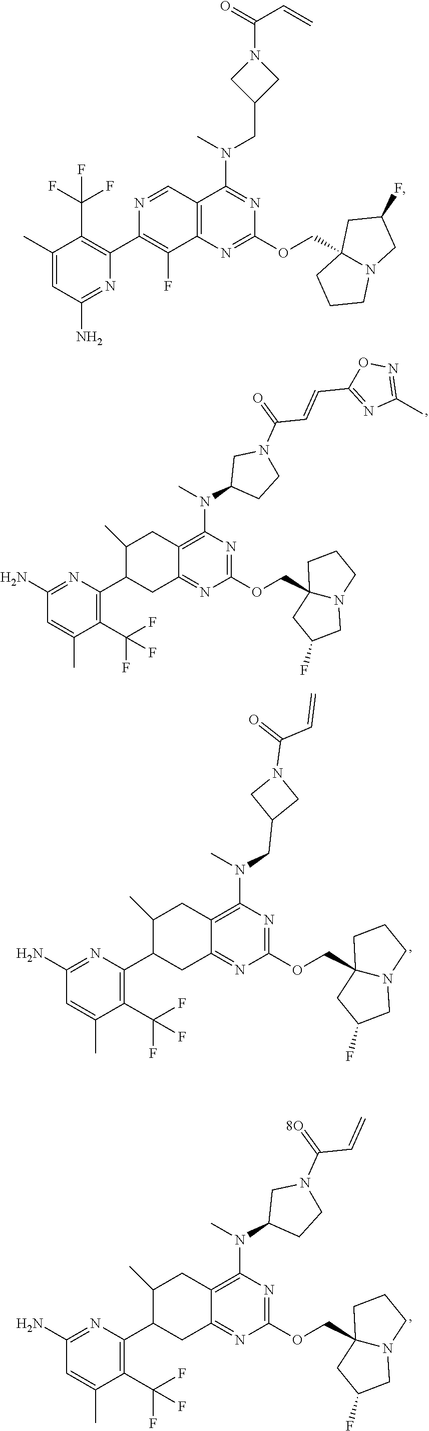

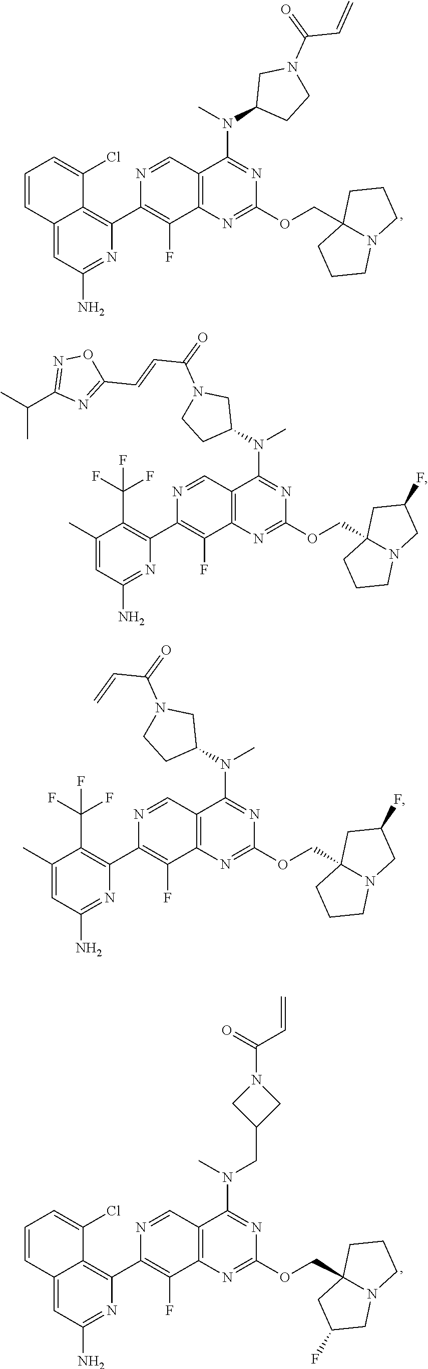 [SMILES: C=CC(=O)N1CC[C@@H](N(C)c2nc(OCC34CCCN3CCC4)nc3c(F)c(-c4nc(N)cc5cccc(Cl)c45)ncc23)C1 ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F ; C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1 ; C=CC(=O)N1CC(CN(C)c2nc(OC[C@@]34CCCN3C[C@H](F)C4)nc3c(F)c(-c4nc(N)cc5cccc(Cl)c45)ncc23)C1]

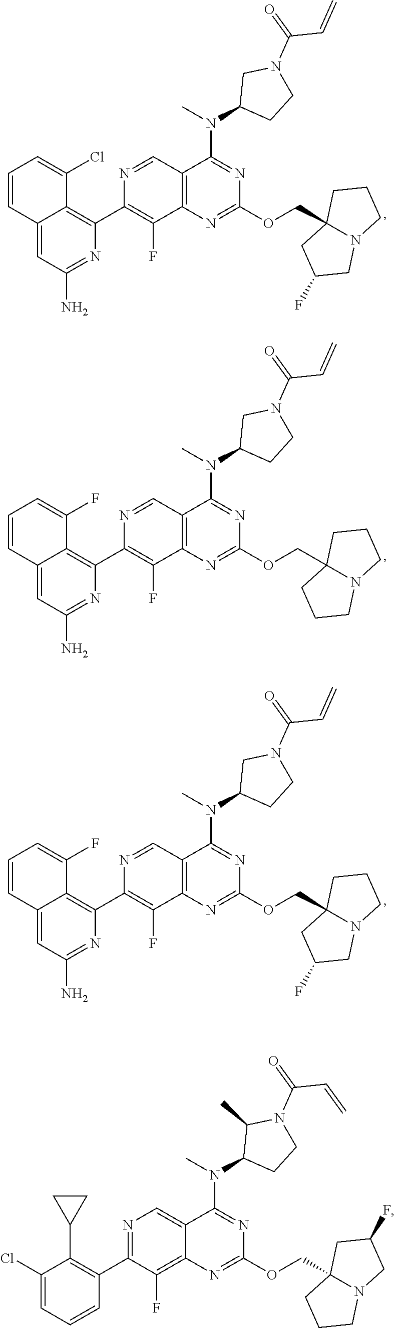 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OCC34CCCN3CCC4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C]

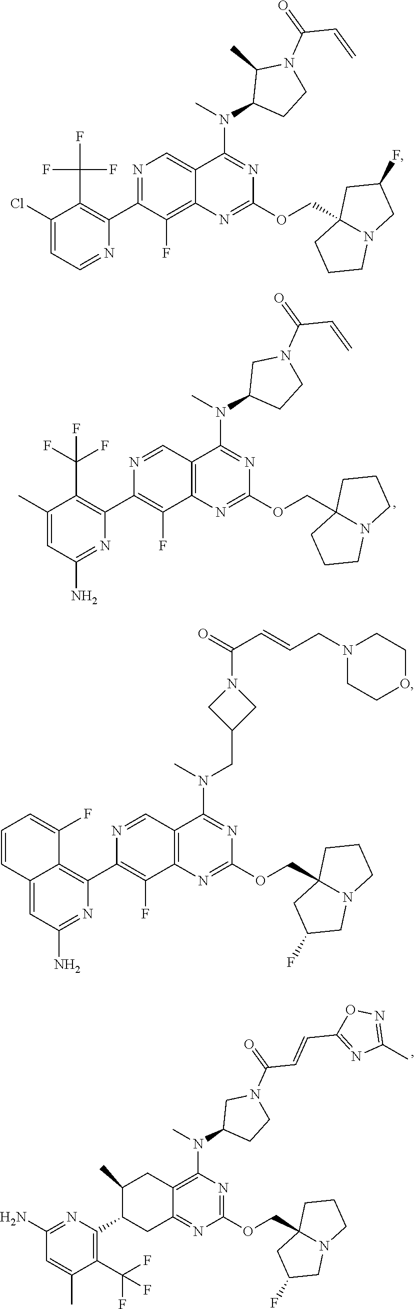 [SMILES: C=CC(=O)N1CC[C@@H](N(C)c2nc(OC[C@@]34CCCN3C[C@H](F)C4)nc3c(F)c(-c4nccc(Cl)c4C(F)(F)F)ncc23)[C@H]1C ; C=CC(=O)N1CC[C@@H](N(C)c2nc(OCC34CCCN3CCC4)nc3c(F)c(-c4nc(N)cc(C)c4C(F)(F)F)ncc23)C1 ; CN(CC1CN(C(=O)/C=C/CN2CCOCC2)C1)c1nc(OC[C@@]23CCCN2C[C@H](F)C3)nc2c(F)c(-c3nc(N)cc4cccc(F)c34)ncc12 ; Cc1noc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1]

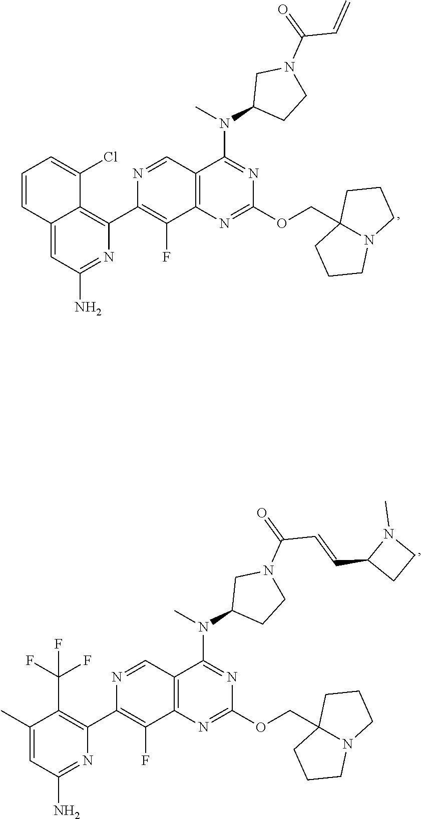 [SMILES: C=CC(=O)N1CC[C@@H](N(C)c2nc(OCC34CCCN3CCC4)nc3c(F)c(-c4nc(N)cc5cccc(Cl)c45)ncc23)C1 ; Cc1cc(N)nc(-c2ncc3c(N(C)[C@@H]4CCN(C(=O)/C=C/[C@@H]5CCN5C)C4)nc(OCC45CCCN4CCC5)nc3c2F)c1C(F)(F)F]

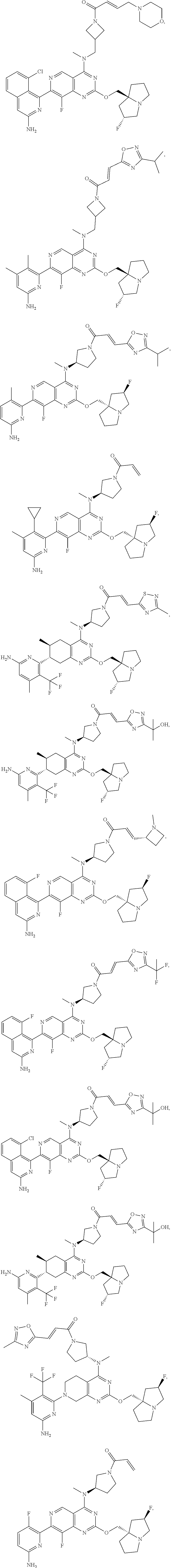

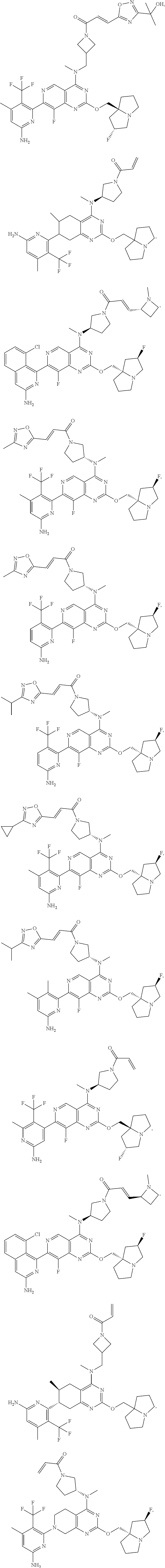

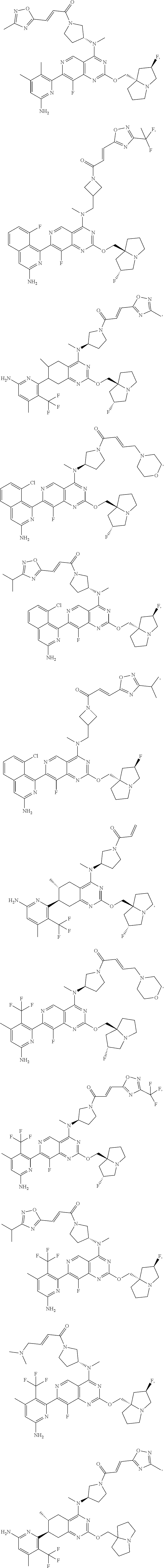

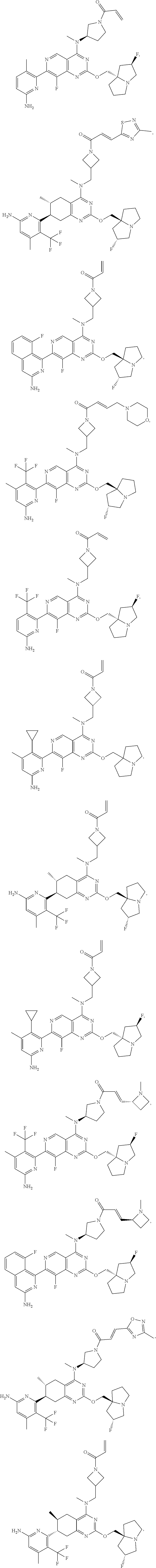

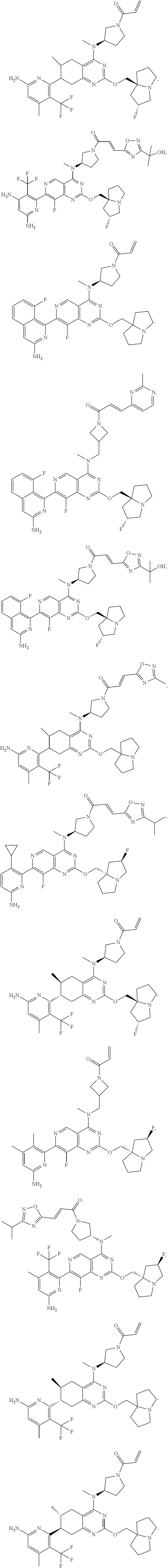

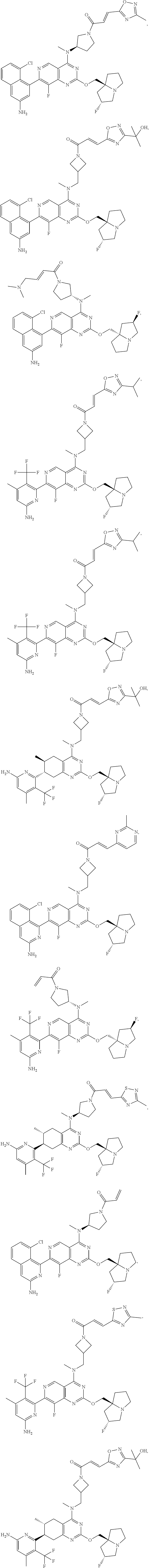

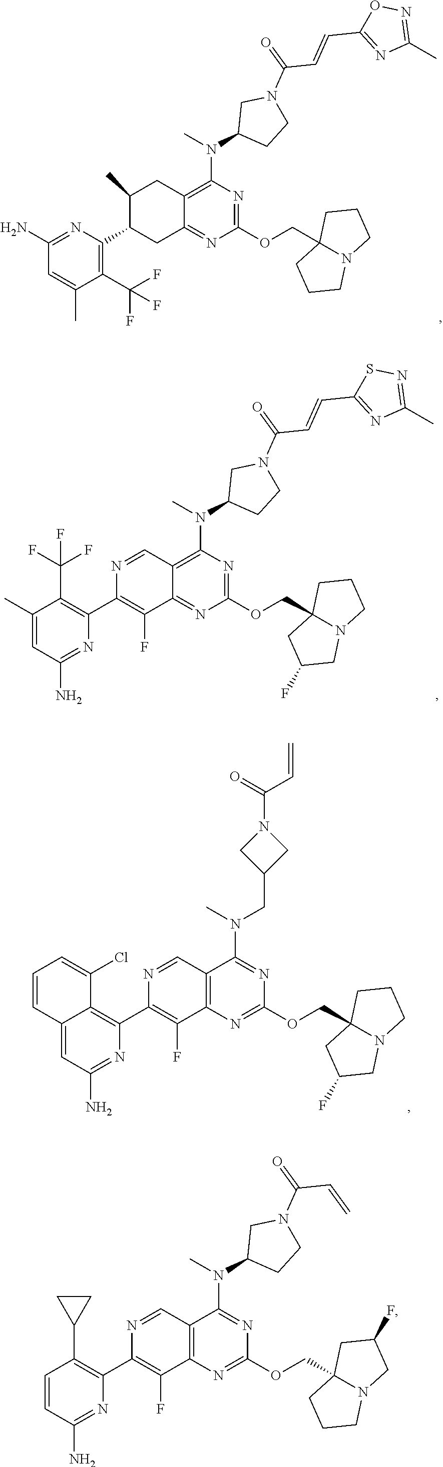

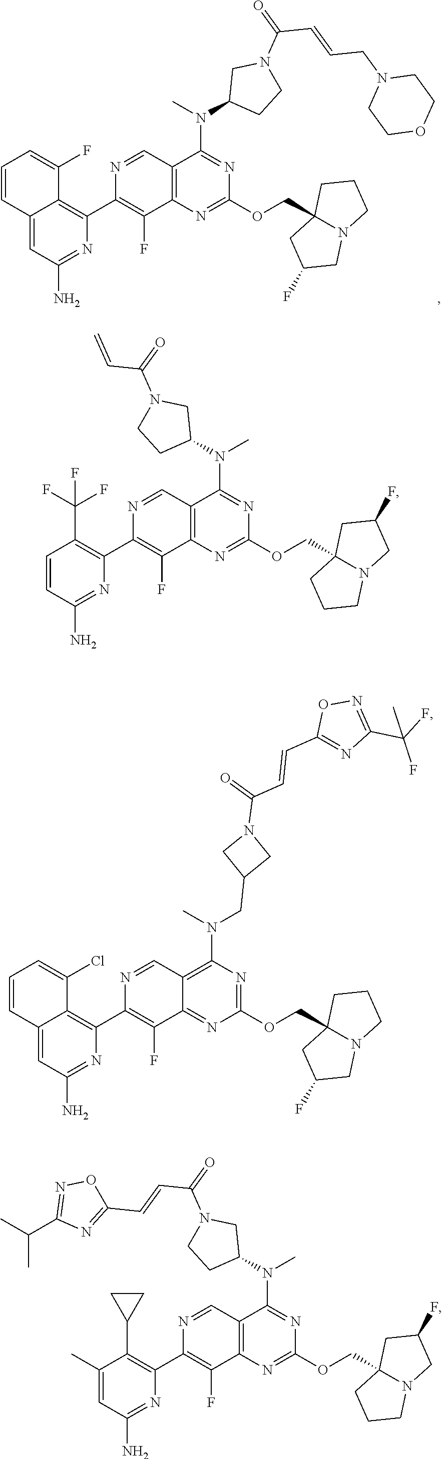 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1 ; C=CC(=O)N1CC[C@@H](N(C)c2nc(OC[C@@]34CCCN3C[C@H](F)C4)nc3c(F)c(-c4nc(N)ccc4C(F)(F)F)ncc23)C1 ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12 ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1]

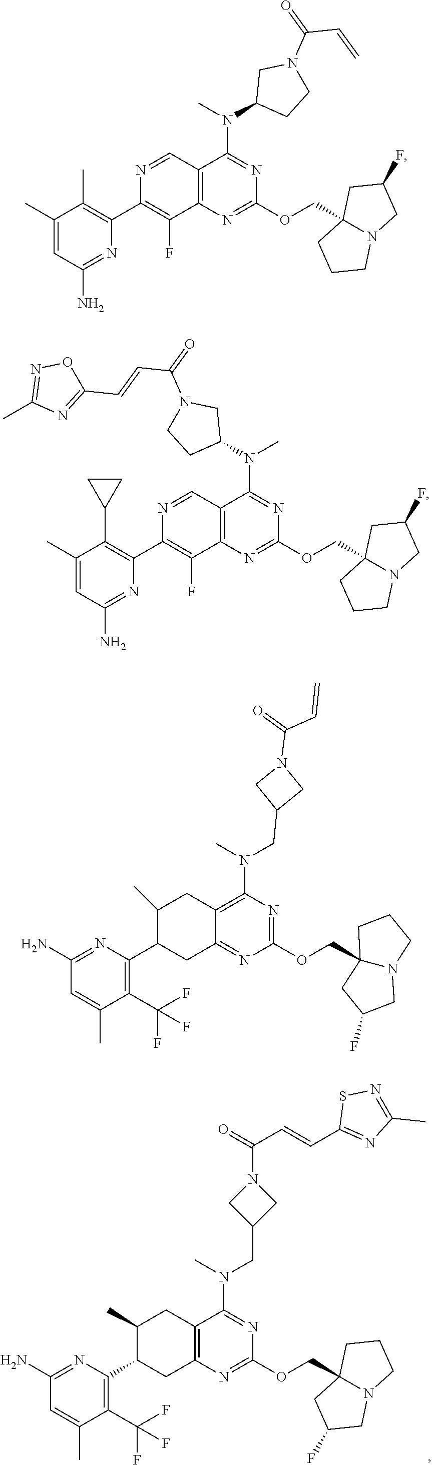 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1 ; C=CC(=O)N1CC(CN(C)c2nc(OC[C@@]34CCCN3C[C@H](F)C4)nc3c2CC(C)C(c2nc(N)cc(C)c2C(F)(F)F)C3)C1 ; Cc1nsc(/C=C/C(=O)N2CC(CN(C)c3nc(OC[C@@]45CCCN4C[C@H](F)C5)nc4c3C[C@H](C)[C@@H](c3nc(N)cc(C)c3C(F)(F)F)C4)C2)n1]

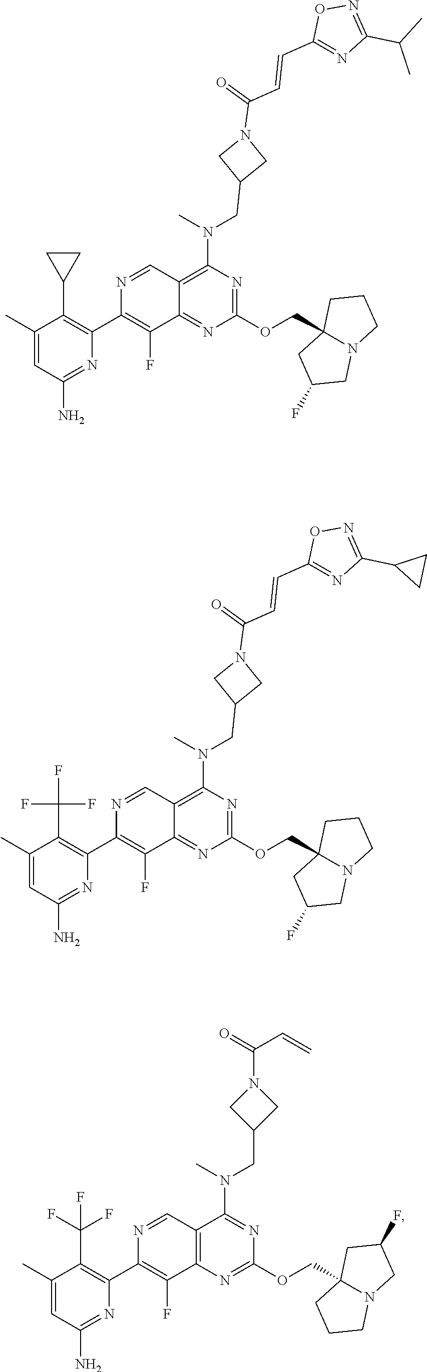

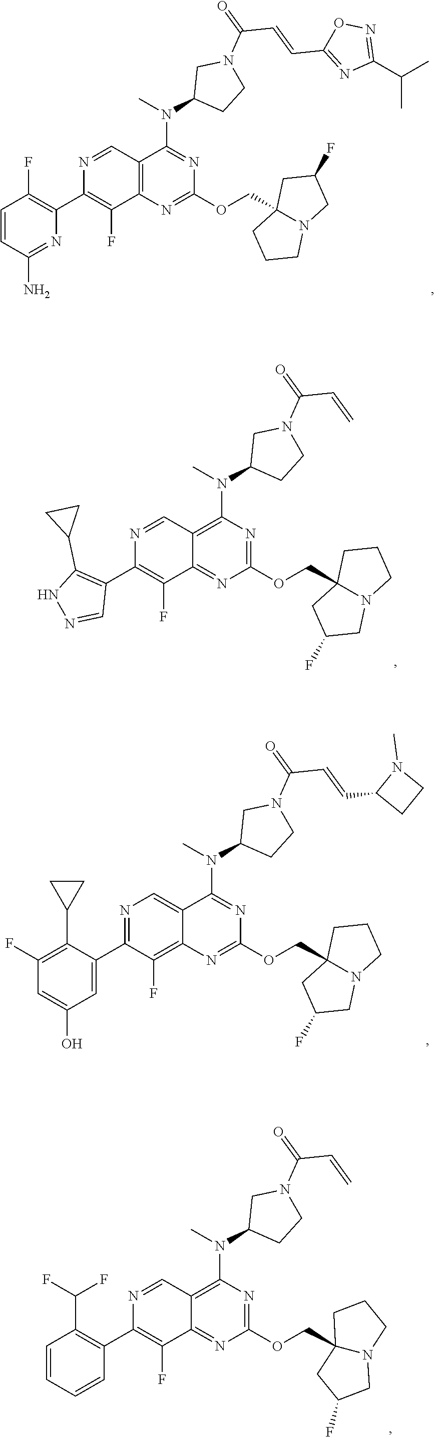 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5F)ncc34)C2)n1 ; C=CC(=O)N1CC[C@@H](N(C)c2nc(OC[C@@]34CCCN3C[C@H](F)C4)nc3c(F)c(-c4cn[nH]c4C4CC4)ncc23)C1 ; CN1CC[C@@H]1/C=C/C(=O)N1CC[C@@H](N(C)c2nc(OC[C@@]34CCCN3C[C@H](F)C4)nc3c(F)c(-c4cc(O)cc(F)c4C4CC4)ncc23)C1 ; C=CC(=O)N1CC[C@@H](N(C)c2nc(OC[C@@]34CCCN3C[C@H](F)C4)nc3c(F)c(-c4ccccc4C(F)F)ncc23)C1]

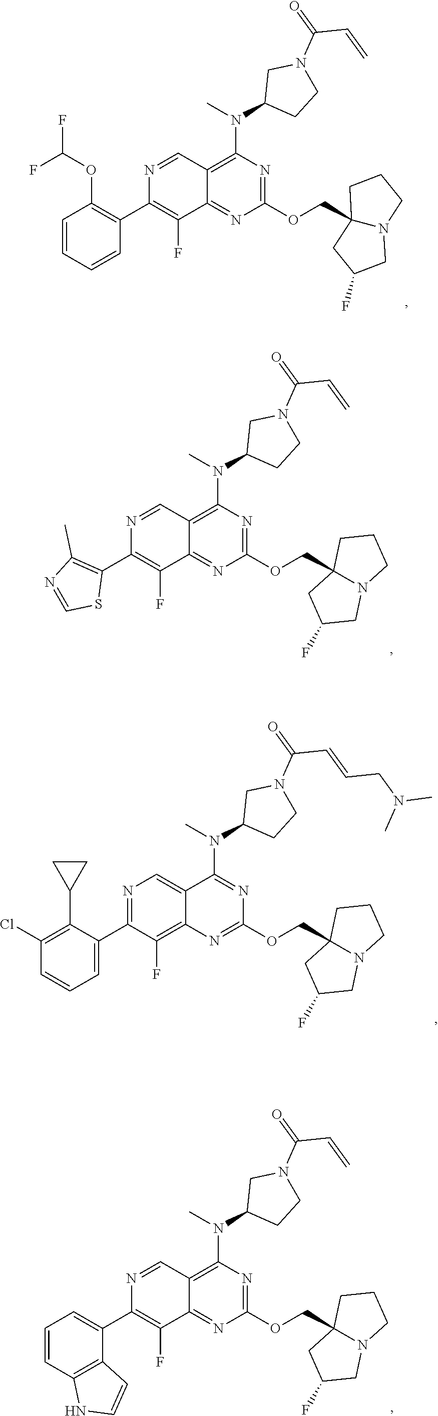 [SMILES: C=CC(=O)N1CC[C@@H](N(C)c2nc(OC[C@@]34CCCN3C[C@H](F)C4)nc3c(F)c(-c4ccccc4OC(F)F)ncc23)C1 ; C=CC(=O)N1CC[C@@H](N(C)c2nc(OC[C@@]34CCCN3C[C@H](F)C4)nc3c(F)c(-c4scnc4C)ncc23)C1 ; CN(C)C/C=C/C(=O)N1CC[C@@H](N(C)c2nc(OC[C@@]34CCCN3C[C@H](F)C4)nc3c(F)c(-c4cccc(Cl)c4C4CC4)ncc23)C1 ; C=CC(=O)N1CC[C@@H](N(C)c2nc(OC[C@@]34CCCN3C[C@H](F)C4)nc3c(F)c(-c4cccc5[nH]ccc45)ncc23)C1]

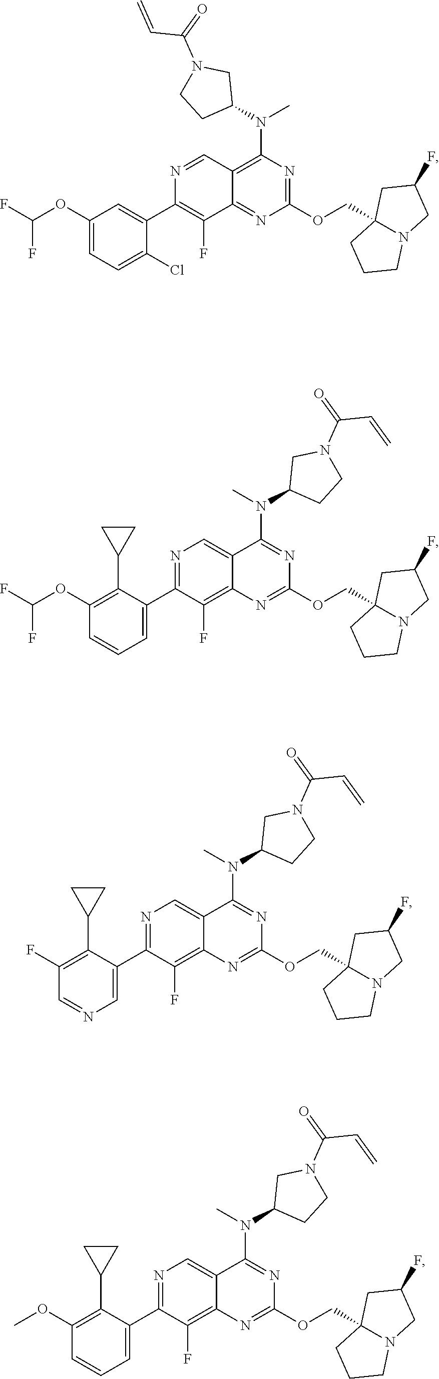

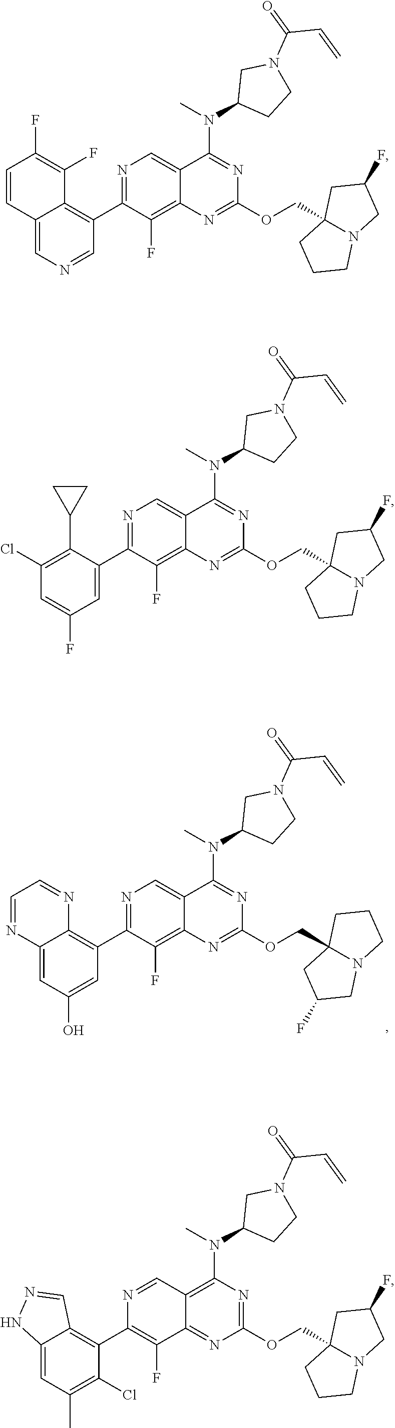 [SMILES: C=CC(=O)N1CC[C@@H](N(C)c2nc(OC[C@@]34CCCN3C[C@H](F)C4)nc3c(F)c(-c4cncc5ccc(F)c(F)c45)ncc23)C1 ; C=CC(=O)N1CC[C@@H](N(C)c2nc(OC[C@@]34CCCN3C[C@H](F)C4)nc3c(F)c(-c4cc(F)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cc(O)cc5nccnc45)ncc23)C1 ; C=CC(=O)N1CC[C@@H](N(C)c2nc(OC[C@@]34CCCN3C[C@H](F)C4)nc3c(F)c(-c4c(Cl)c(C)cc5[nH]ncc45)ncc23)C1]

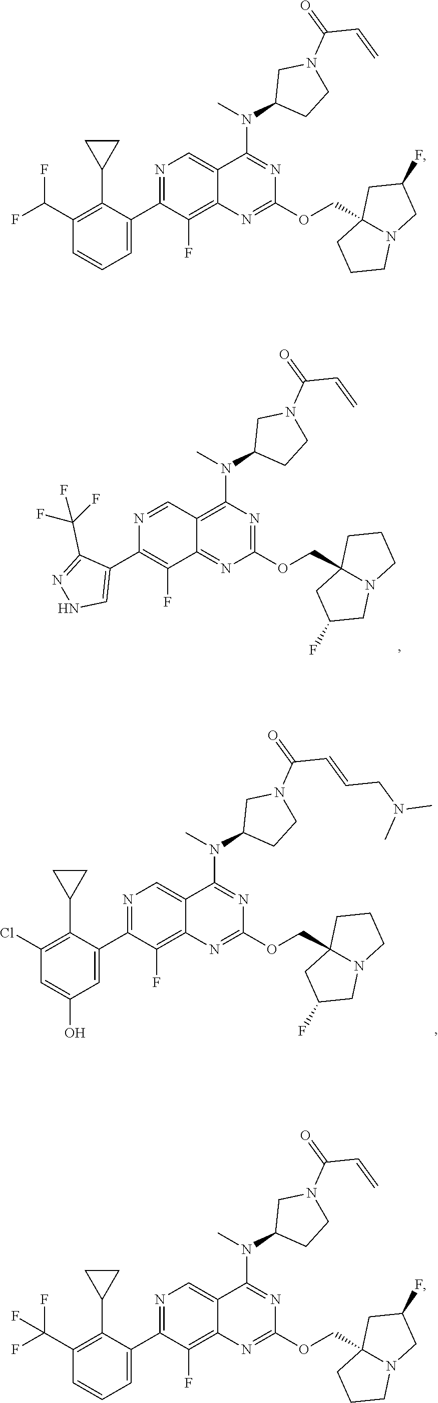

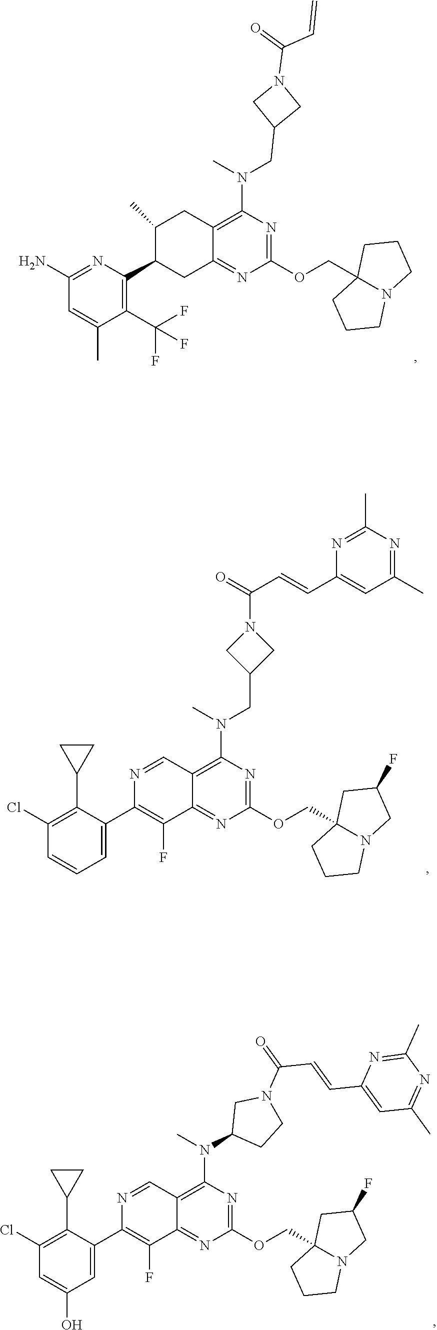 [SMILES: C=CC(=O)N1CC(CN(C)c2nc(OCC34CCCN3CCC4)nc3c2C[C@@H](C)[C@H](c2nc(N)cc(C)c2C(F)(F)F)C3)C1 ; Cc1cc(/C=C/C(=O)N2CC(CN(C)c3nc(OC[C@@]45CCCN4C[C@H](F)C5)nc4c(F)c(-c5cccc(Cl)c5C5CC5)ncc34)C2)nc(C)n1 ; Cc1cc(/C=C/C(=O)N2CC[C@@H](N(C)c3nc(OC[C@@]45CCCN4C[C@H](F)C5)nc4c(F)c(-c5cc(O)cc(Cl)c5C5CC5)ncc34)C2)nc(C)n1]

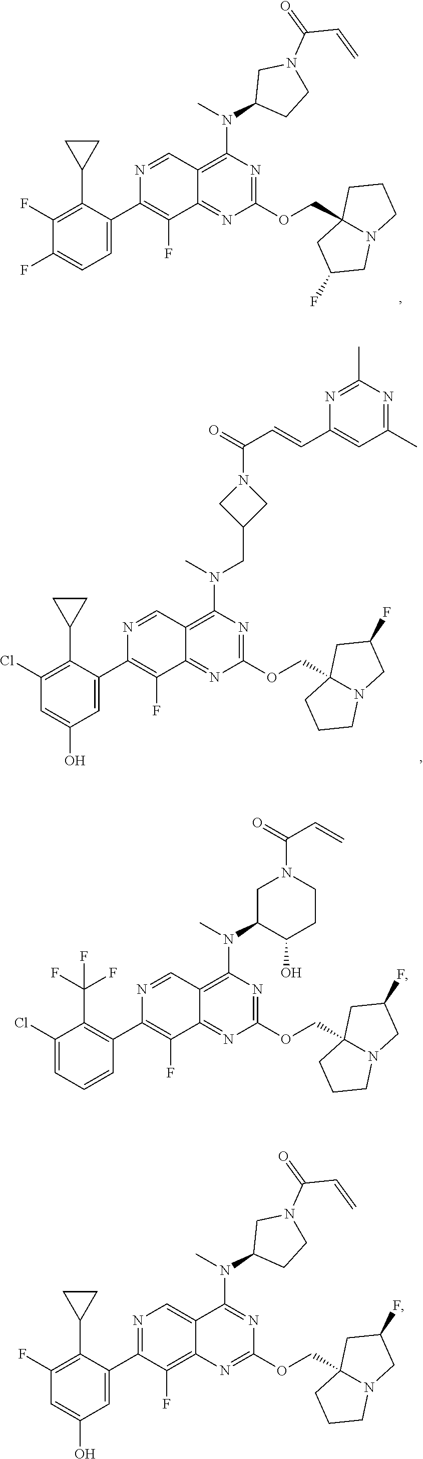 [SMILES: C=CC(=O)N1CC[C@@H](N(C)c2nc(OC[C@@]34CCCN3C[C@H](F)C4)nc3c(F)c(-c4ccc(F)c(F)c4C4CC4)ncc23)C1 ; Cc1cc(/C=C/C(=O)N2CC(CN(C)c3nc(OC[C@@]45CCCN4C[C@H](F)C5)nc4c(F)c(-c5cc(O)cc(Cl)c5C5CC5)ncc34)C2)nc(C)n1 ; C=CC(=O)N1CC[C@H](O)[C@@H](N(C)c2nc(OC[C@@]34CCCN3C[C@H](F)C4)nc3c(F)c(-c4cccc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(O)cc(F)c4C4CC4)ncc23)C1]

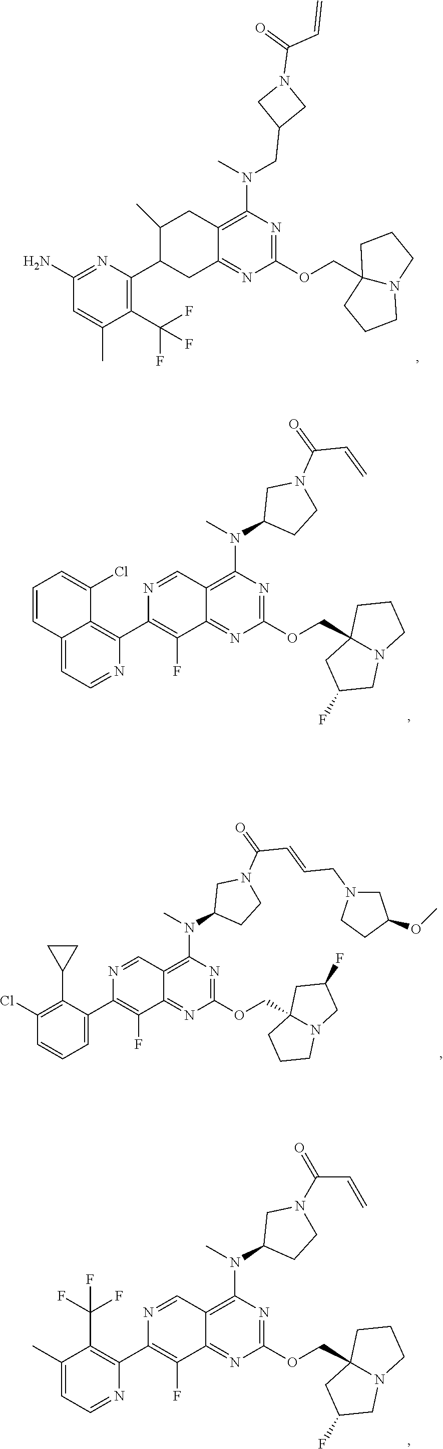

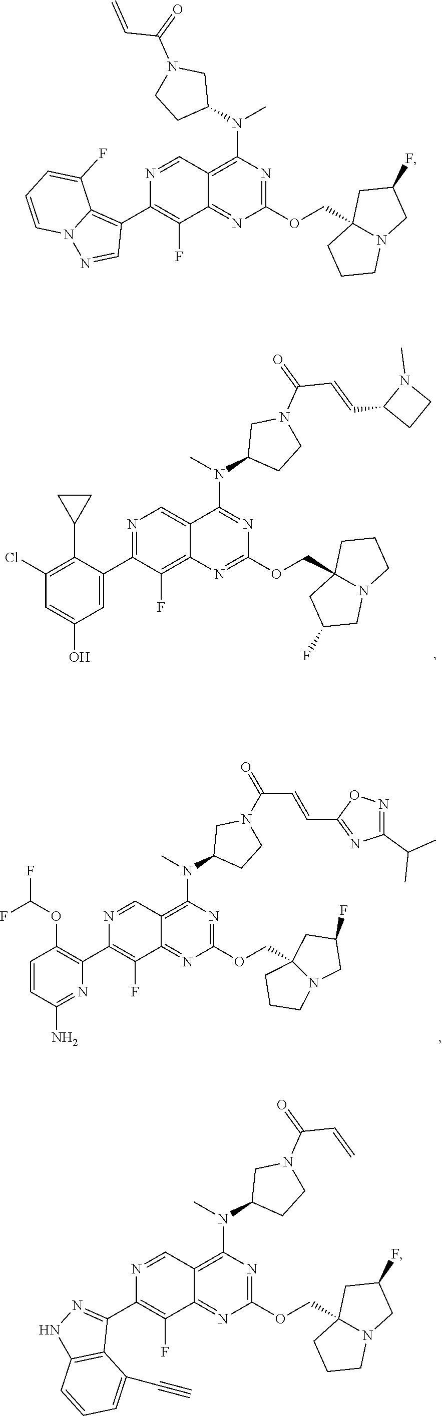

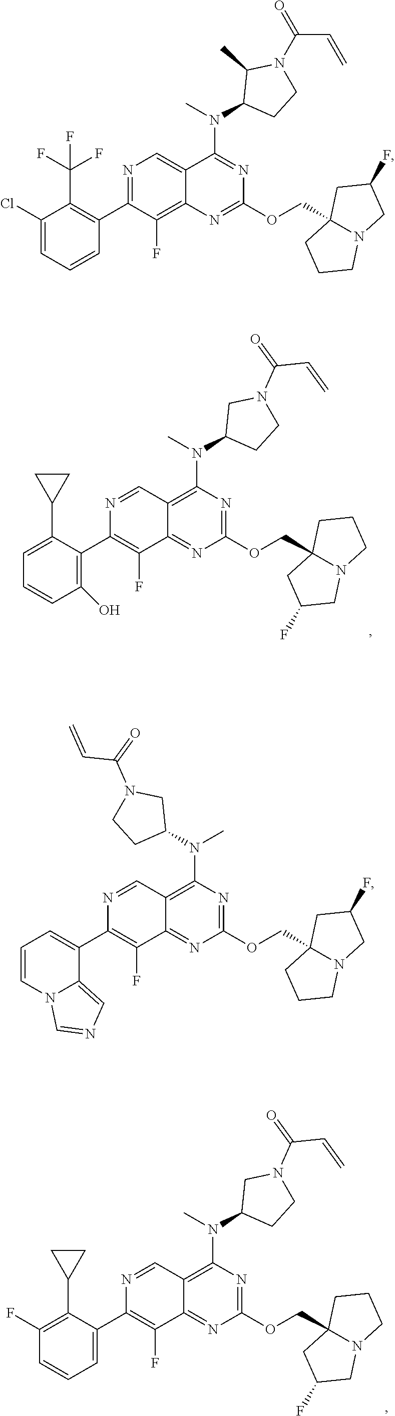 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OC[C@@]34CCCN3C[C@H](F)C4)nc3c(F)c(-c4c(O)cccc4C4CC4)ncc23)C1 ; C=CC(=O)N1CC[C@@H](N(C)c2nc(OC[C@@]34CCCN3C[C@H](F)C4)nc3c(F)c(-c4cccn5cncc45)ncc23)C1 ; C=CC(=O)N1CC[C@@H](N(C)c2nc(OC[C@@]34CCCN3C[C@H](F)C4)nc3c(F)c(-c4cccc(F)c4C4CC4)ncc23)C1]

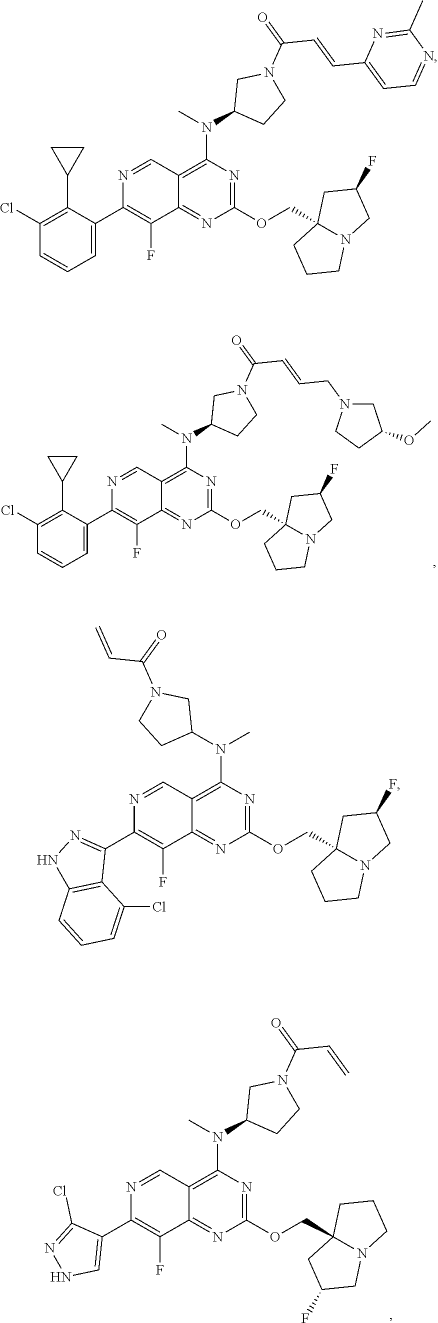 [SMILES: Cc1nccc(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)n1 ; CO[C@@H]1CCN(C/C=C/C(=O)N2CC[C@@H](N(C)c3nc(OC[C@@]45CCCN4C[C@H](F)C5)nc4c(F)c(-c5cccc(Cl)c5C5CC5)ncc34)C2)C1 ; C=CC(=O)N1CCC(N(C)c2nc(OC[C@@]34CCCN3C[C@H](F)C4)nc3c(F)c(-c4n[nH]c5cccc(Cl)c45)ncc23)C1 ; C=CC(=O)N1CC[C@@H](N(C)c2nc(OC[C@@]34CCCN3C[C@H](F)C4)nc3c(F)c(-c4c[nH]nc4Cl)ncc23)C1]

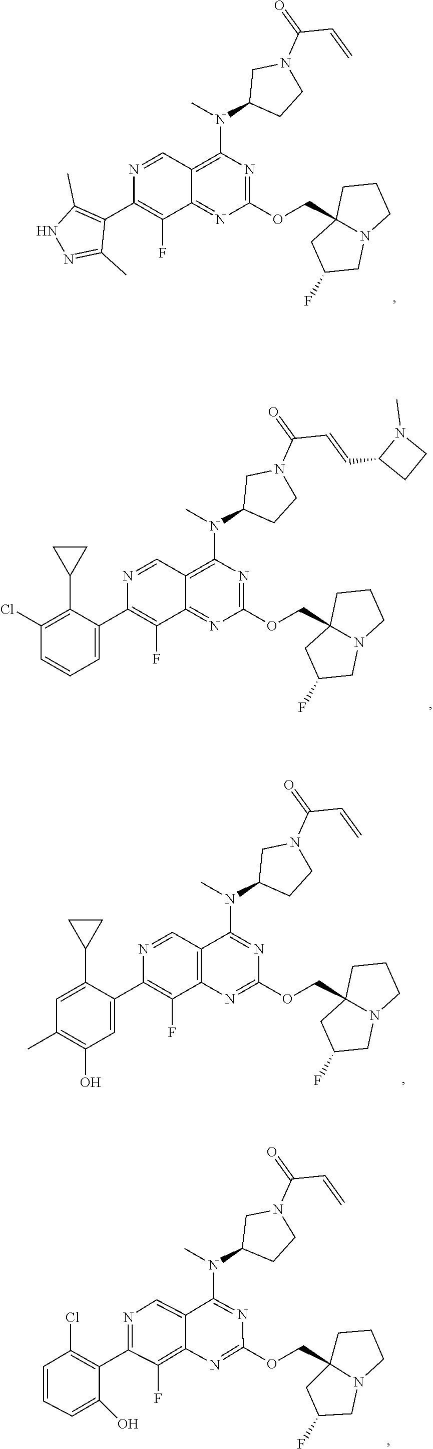

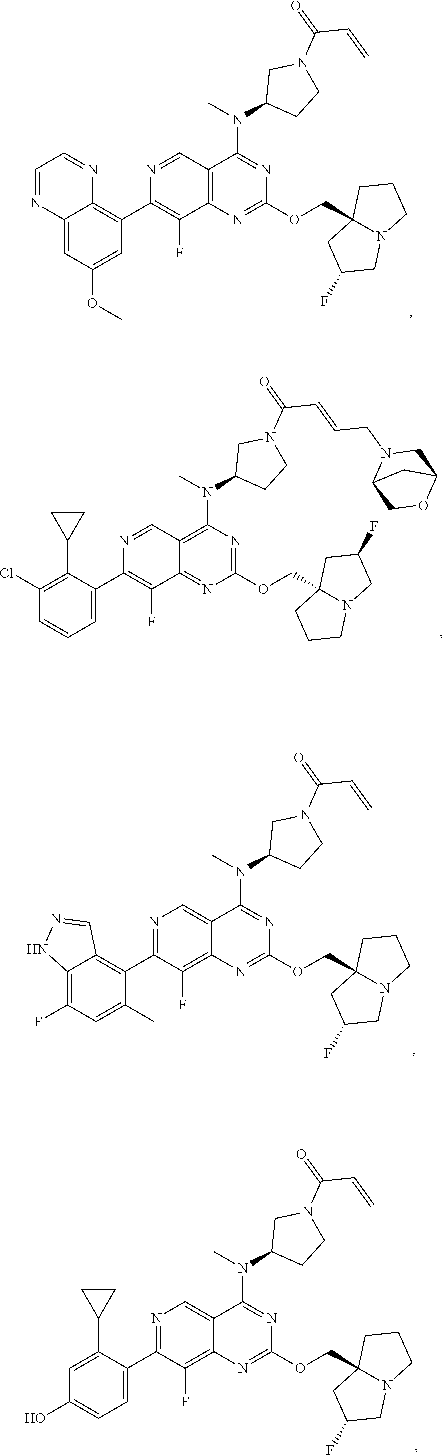 [SMILES: C=CC(=O)N1CC[C@@H](N(C)c2nc(OC[C@@]34CCCN3C[C@H](F)C4)nc3c(F)c(-c4cc(OC)cc5nccnc45)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@@H]3C[C@H]2CO3)C1 ; C=CC(=O)N1CC[C@@H](N(C)c2nc(OC[C@@]34CCCN3C[C@H](F)C4)nc3c(F)c(-c4c(C)cc(F)c5[nH]ncc45)ncc23)C1 ; C=CC(=O)N1CC[C@@H](N(C)c2nc(OC[C@@]34CCCN3C[C@H](F)C4)nc3c(F)c(-c4ccc(O)cc4C4CC4)ncc23)C1]

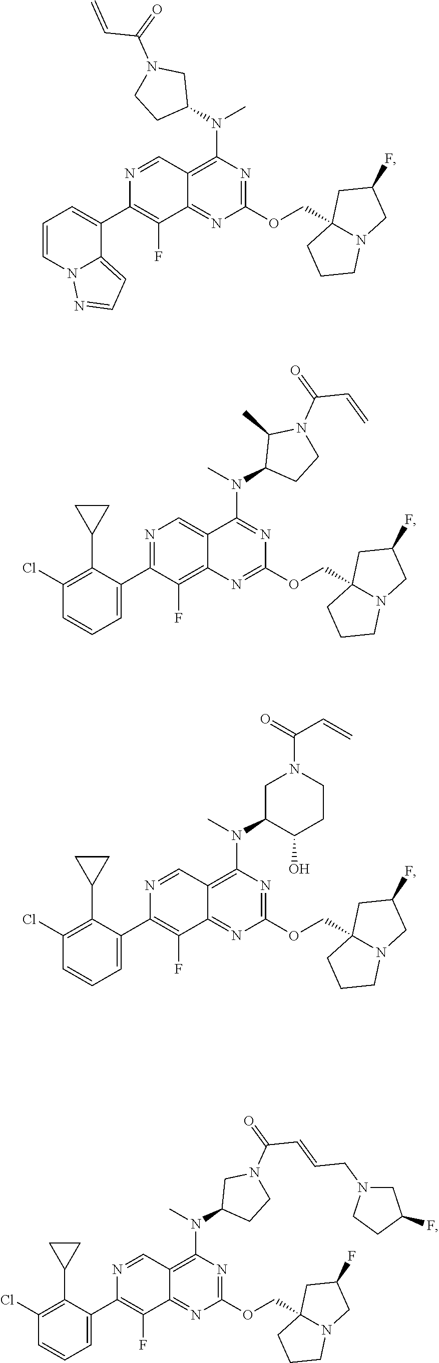 [SMILES: C=CC(=O)N1CC[C@@H](N(C)c2nc(OC[C@@]34CCCN3C[C@H](F)C4)nc3c(F)c(-c4cccn5nccc45)ncc23)C1 ; C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C ; C=CC(=O)N1CC[C@H](O)[C@@H](N(C)c2nc(OC[C@@]34CCCN3C[C@H](F)C4)nc3c(F)c(-c4cccc(Cl)c4C4CC4)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@H](F)C2)C1]

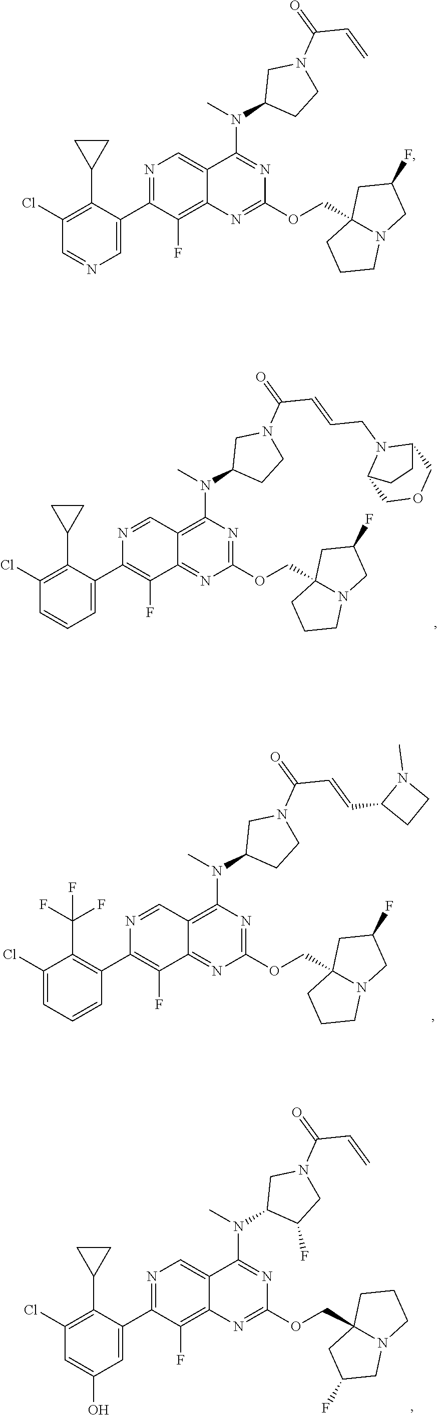 [SMILES: C=CC(=O)N1CC[C@@H](N(C)c2nc(OC[C@@]34CCCN3C[C@H](F)C4)nc3c(F)c(-c4cncc(Cl)c4C4CC4)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2[C@@H]3CC[C@H]2COC3)C1 ; CN1CC[C@@H]1/C=C/C(=O)N1CC[C@@H](N(C)c2nc(OC[C@@]34CCCN3C[C@H](F)C4)nc3c(F)c(-c4cccc(Cl)c4C(F)(F)F)ncc23)C1 ; C=CC(=O)N1C[C@@H](N(C)c2nc(OC[C@@]34CCCN3C[C@H](F)C4)nc3c(F)c(-c4cc(O)cc(Cl)c4C4CC4)ncc23)[C@@H](F)C1]

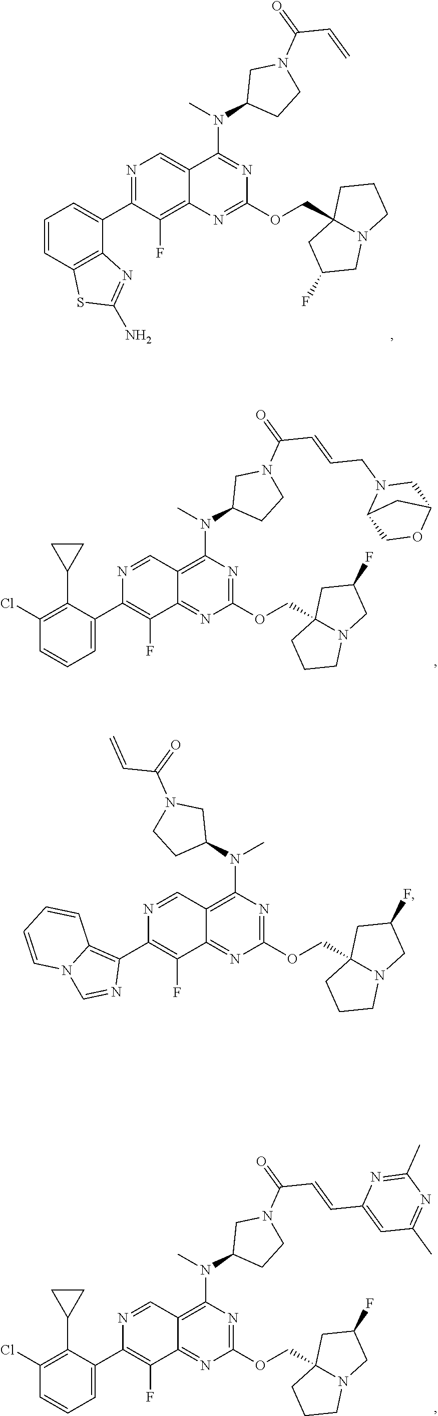 [SMILES: C=CC(=O)N1CC[C@@H](N(C)c2nc(OC[C@@]34CCCN3C[C@H](F)C4)nc3c(F)c(-c4cccc5sc(N)nc45)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@H]3C[C@@H]2CO3)C1 ; C=CC(=O)N1CC[C@H](N(C)c2nc(OC[C@@]34CCCN3C[C@H](F)C4)nc3c(F)c(-c4ncn5ccccc45)ncc23)C1 ; Cc1cc(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1]

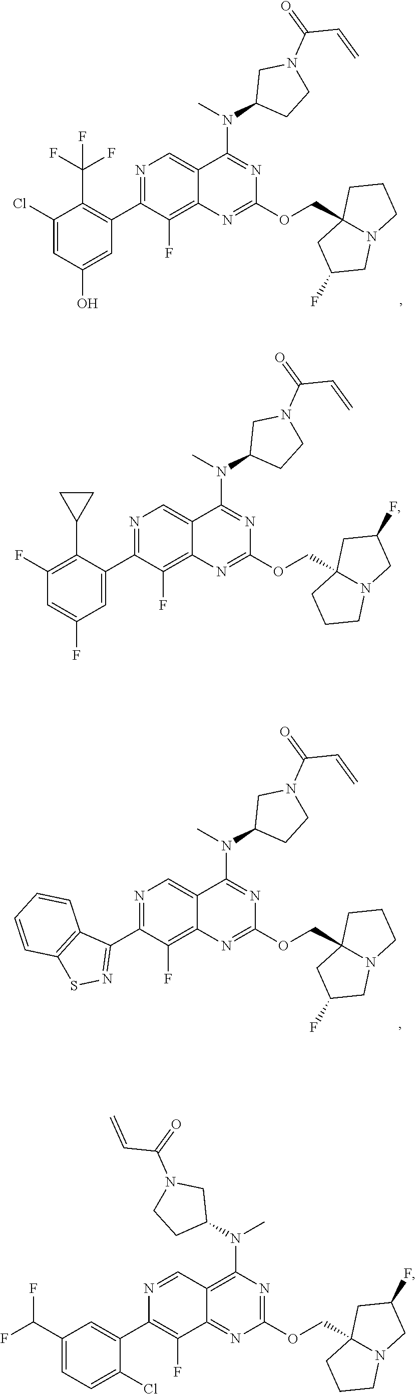 [SMILES: C=CC(=O)N1CC[C@@H](N(C)c2nc(OC[C@@]34CCCN3C[C@H](F)C4)nc3c(F)c(-c4cc(O)cc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(F)cc(F)c4C4CC4)ncc23)C1 ; C=CC(=O)N1CC[C@@H](N(C)c2nc(OC[C@@]34CCCN3C[C@H](F)C4)nc3c(F)c(-c4nsc5ccccc45)ncc23)C1 ; C=CC(=O)N1CC[C@@H](N(C)c2nc(OC[C@@]34CCCN3C[C@H](F)C4)nc3c(F)c(-c4cc(C(F)F)ccc4Cl)ncc23)C1]

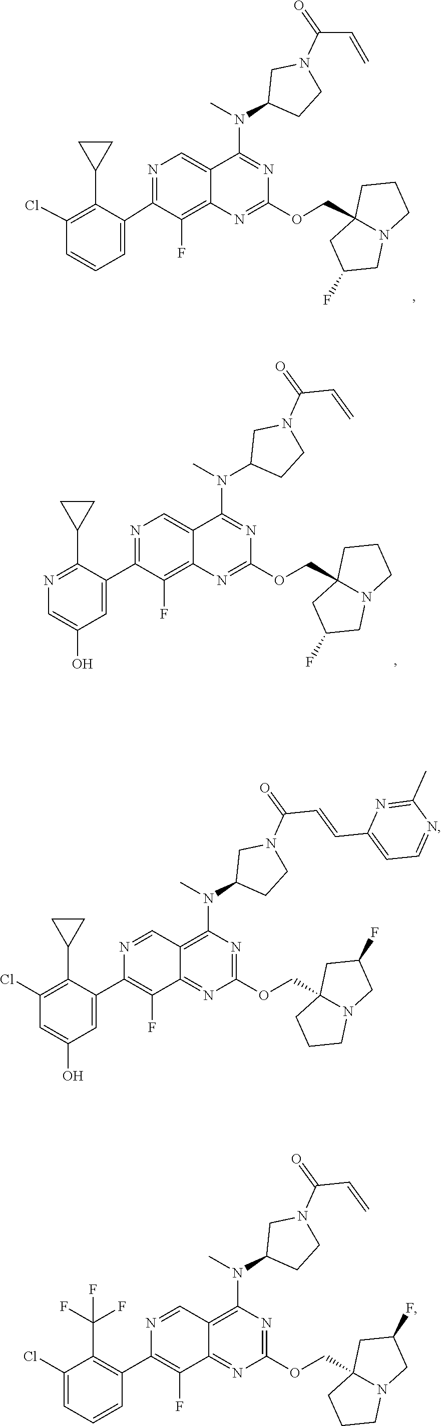 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)C1 ; C=CC(=O)N1CCC(N(C)c2nc(OC[C@@]34CCCN3C[C@H](F)C4)nc3c(F)c(-c4cc(O)cnc4C4CC4)ncc23)C1 ; Cc1nccc(/C=C/C(=O)N2CC[C@@H](N(C)c3nc(OC[C@@]45CCCN4C[C@H](F)C5)nc4c(F)c(-c5cc(O)cc(Cl)c5C5CC5)ncc34)C2)n1 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)C1]

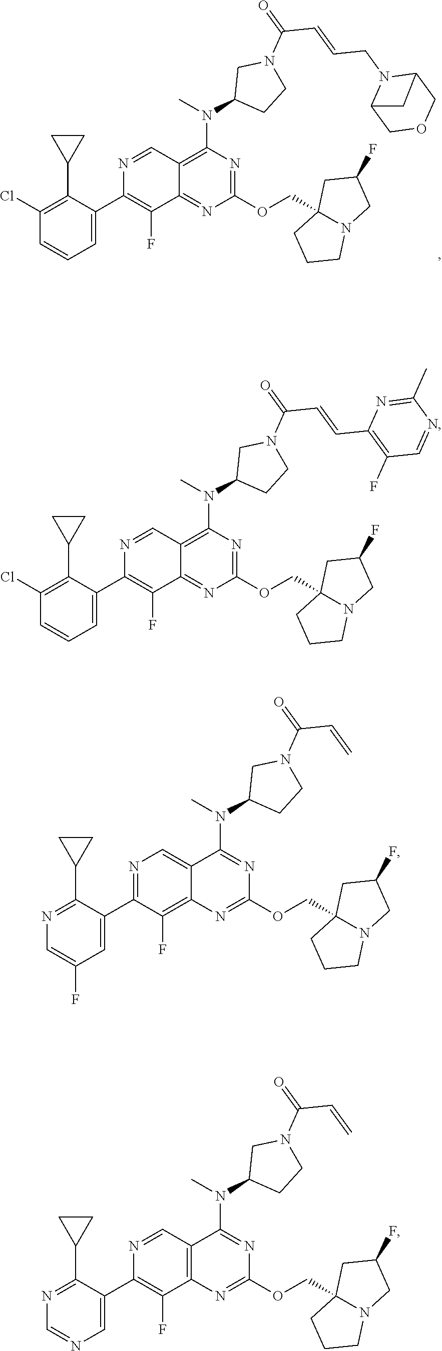 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C3COCC2C3)C1 ; Cc1ncc(F)c(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)n1 ; C=CC(=O)N1CC[C@@H](N(C)c2nc(OC[C@@]34CCCN3C[C@H](F)C4)nc3c(F)c(-c4cc(F)cnc4C4CC4)ncc23)C1 ; C=CC(=O)N1CC[C@@H](N(C)c2nc(OC[C@@]34CCCN3C[C@H](F)C4)nc3c(F)c(-c4cncnc4C4CC4)ncc23)C1]

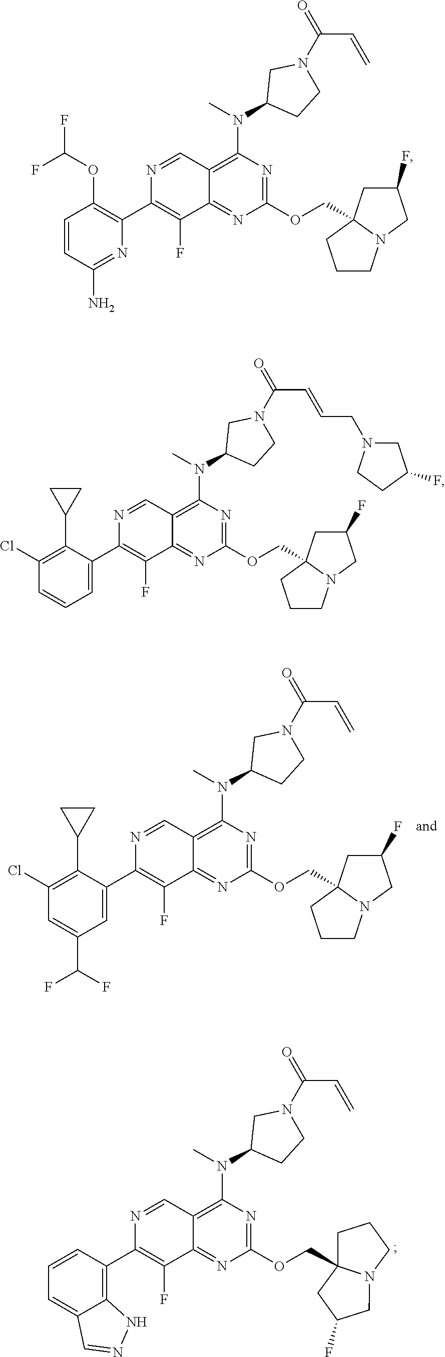 [SMILES: C=CC(=O)N1CC[C@@H](N(C)c2nc(OC[C@@]34CCCN3C[C@H](F)C4)nc3c(F)c(-c4nc(N)ccc4OC(F)F)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@@H](F)C2)C1 ; C=CC(=O)N1CC[C@@H](N(C)c2nc(OC[C@@]34CCCN3C[C@H](F)C4)nc3c(F)c(-c4cc(C(F)F)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cccc5cn[nH]c45)ncc23)C1]

and all salts and isotopologues thereof.

Embodiment 307. The compound of embodiment 1, selected from the group consisting of:

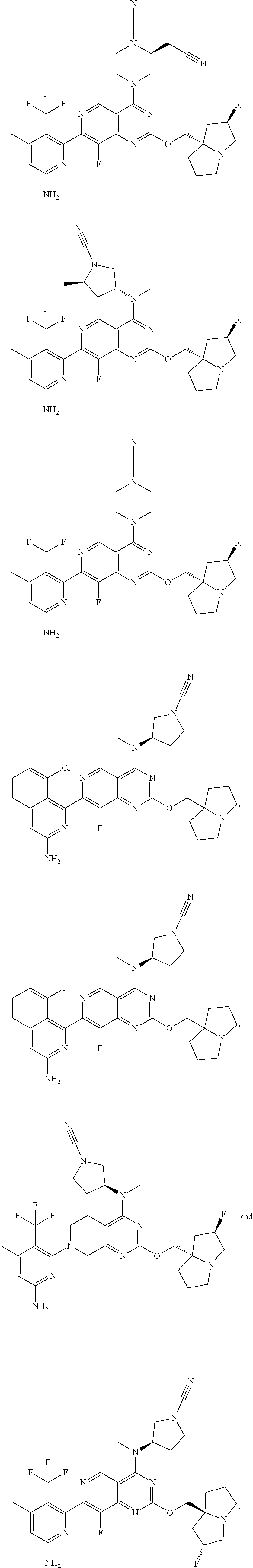

and all salts and isotopologues thereof.

Embodiment 308. The compound of embodiment 1, selected from the group consisting of:

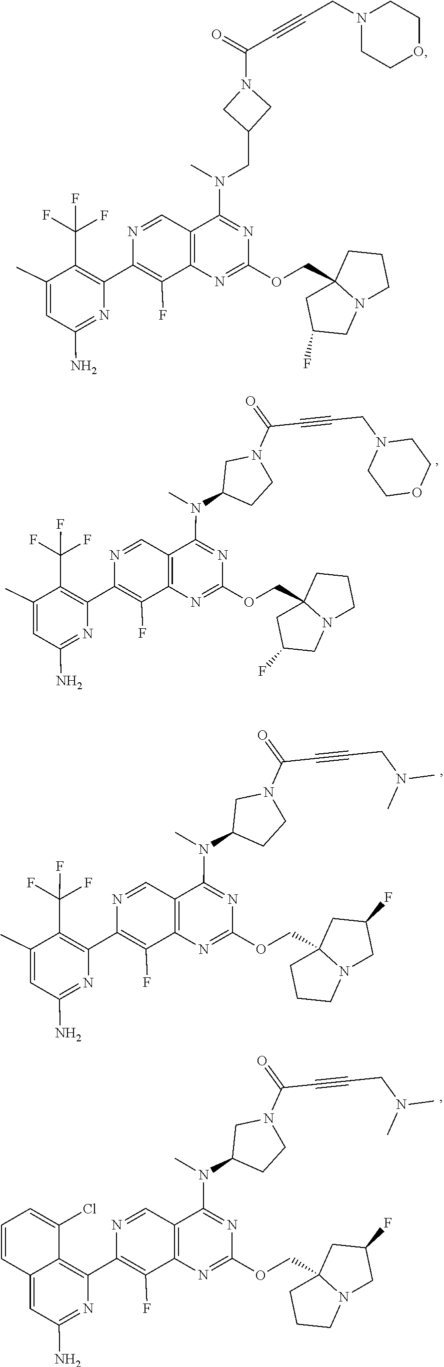

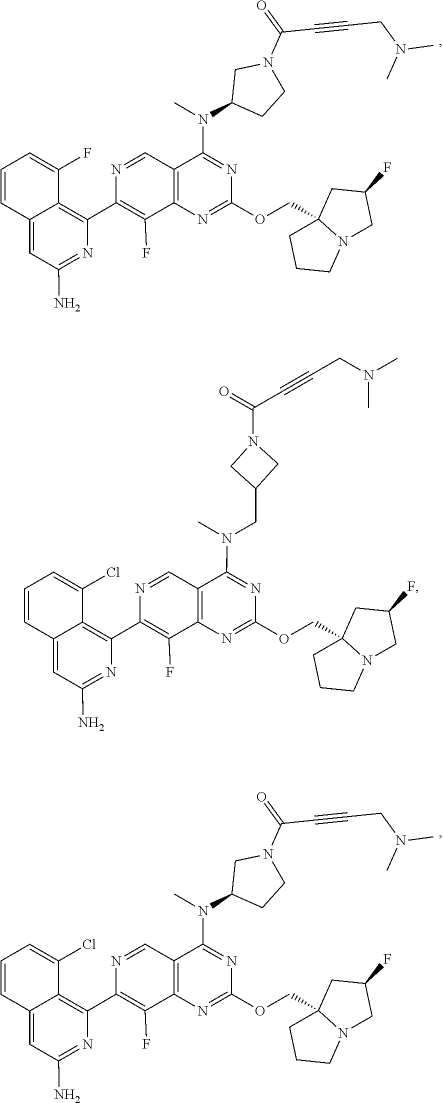 [SMILES: CN(C)CC#CC(=O)N1CC[C@@H](N(C)c2nc(OC[C@@]34CCCN3C[C@H](F)C4)nc3c(F)c(-c4nc(N)cc5cccc(F)c45)ncc23)C1 ; CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1 ; CN(C)CC#CC(=O)N1CC[C@@H](N(C)c2nc(OC[C@@]34CCCN3C[C@H](F)C4)nc3c(F)c(-c4nc(N)cc5cccc(Cl)c45)ncc23)C1]

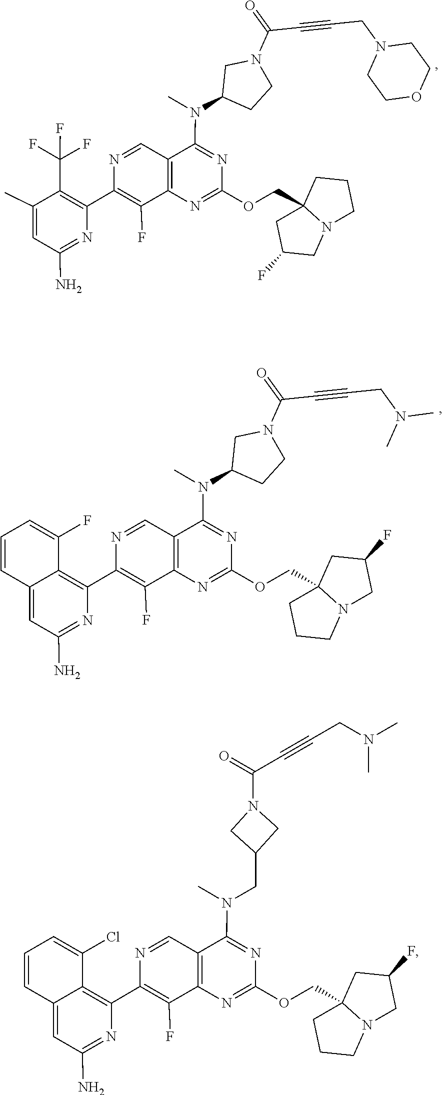 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F ; CN(C)CC#CC(=O)N1CC[C@@H](N(C)c2nc(OC[C@@]34CCCN3C[C@H](F)C4)nc3c(F)c(-c4nc(N)cc5cccc(F)c45)ncc23)C1 ; CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1]

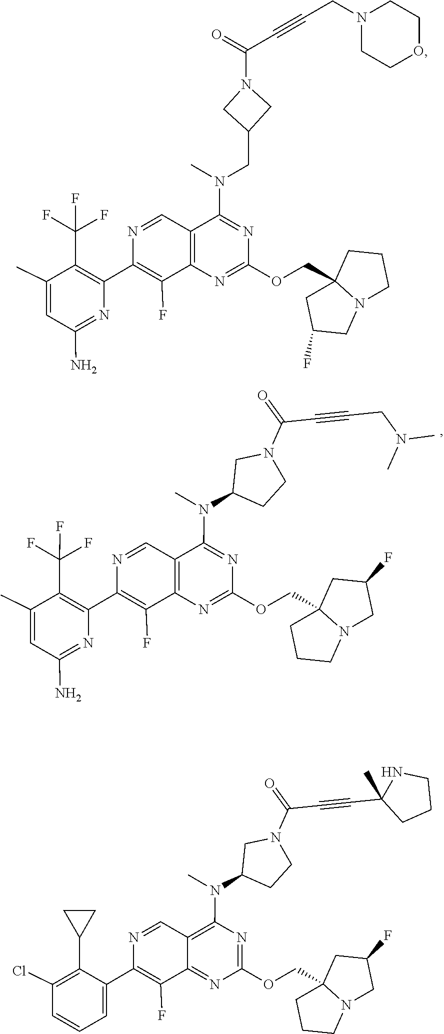

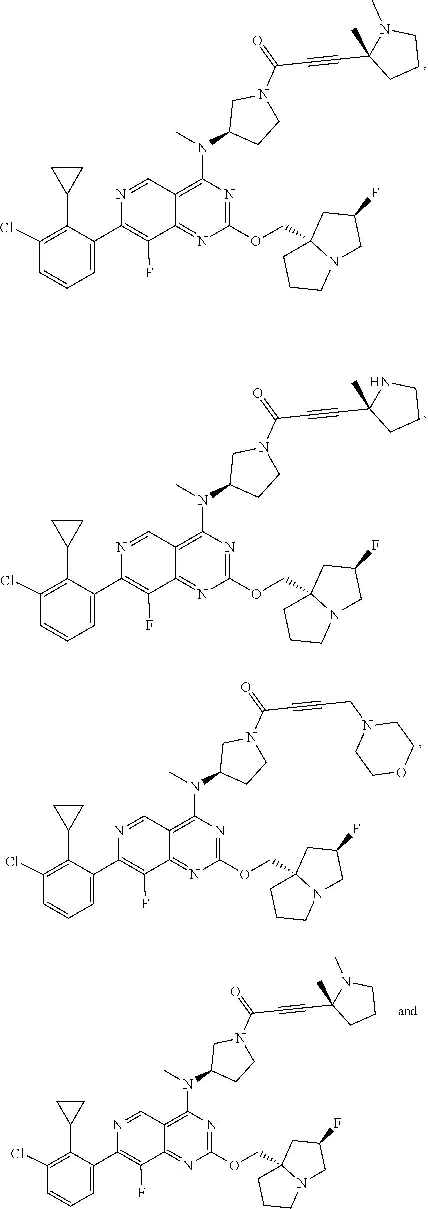 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2C)C1 ; CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2)C1 ; CN(c1nc(OC[C@@]23CCCN2C[C@H](F)C3)nc2c(F)c(-c3cccc(Cl)c3C3CC3)ncc12)[C@@H]1CCN(C(=O)C#CCN2CCOCC2)C1 ; CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2C)C1]

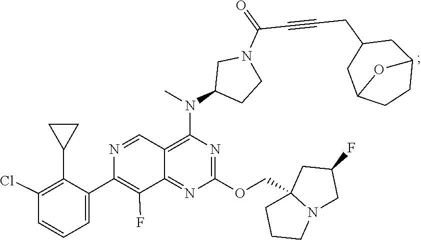 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#CCC2CC3CCC(C2)O3)C1]

and all salts and isotopologues thereof.

Embodiment 309. The compound of embodiment 1, selected from the group consisting of:

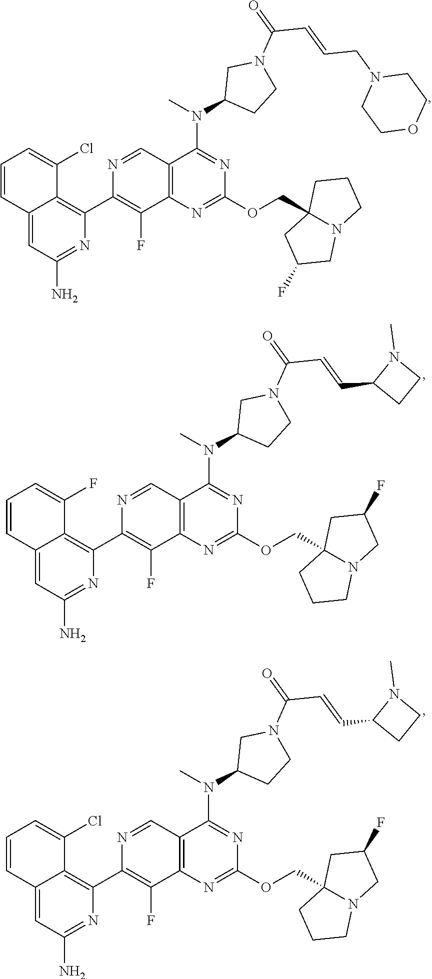

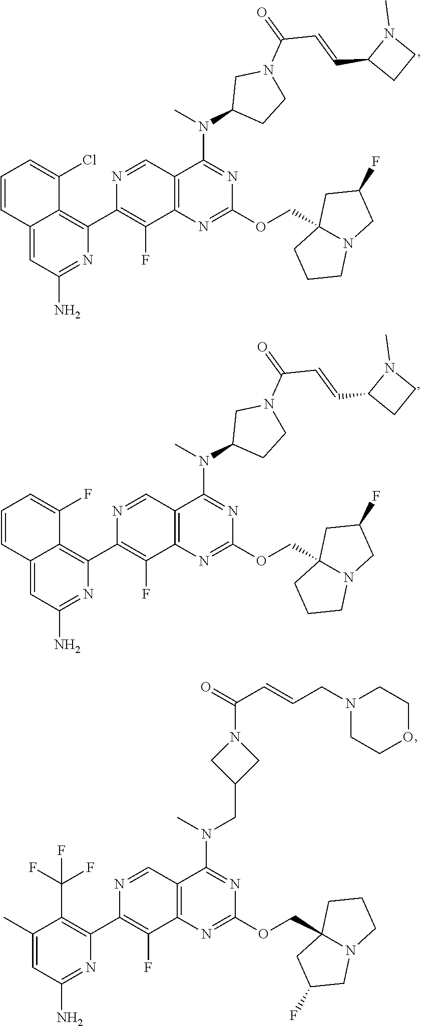 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F]

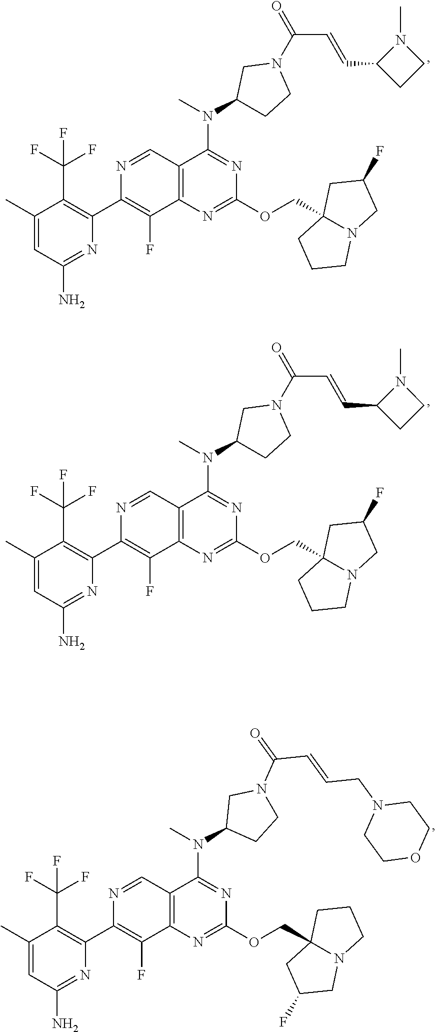 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F]

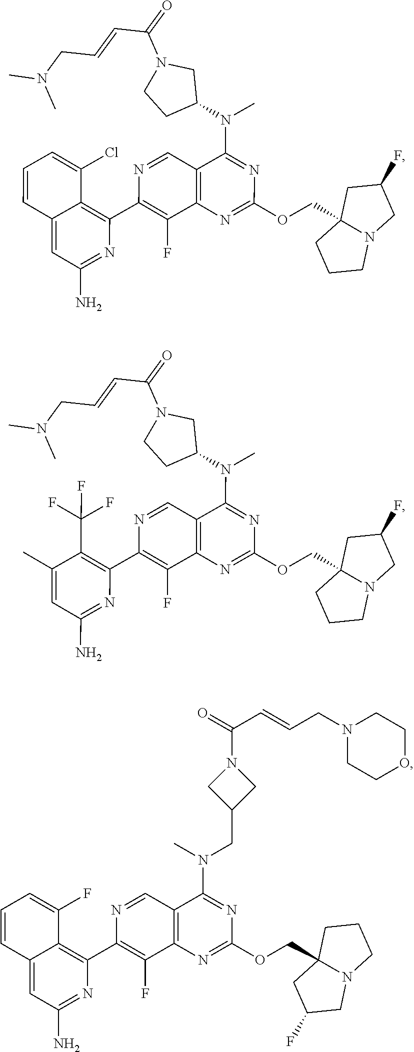 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; CN(CC1CN(C(=O)/C=C/CN2CCOCC2)C1)c1nc(OC[C@@]23CCCN2C[C@H](F)C3)nc2c(F)c(-c3nc(N)cc4cccc(F)c34)ncc12]

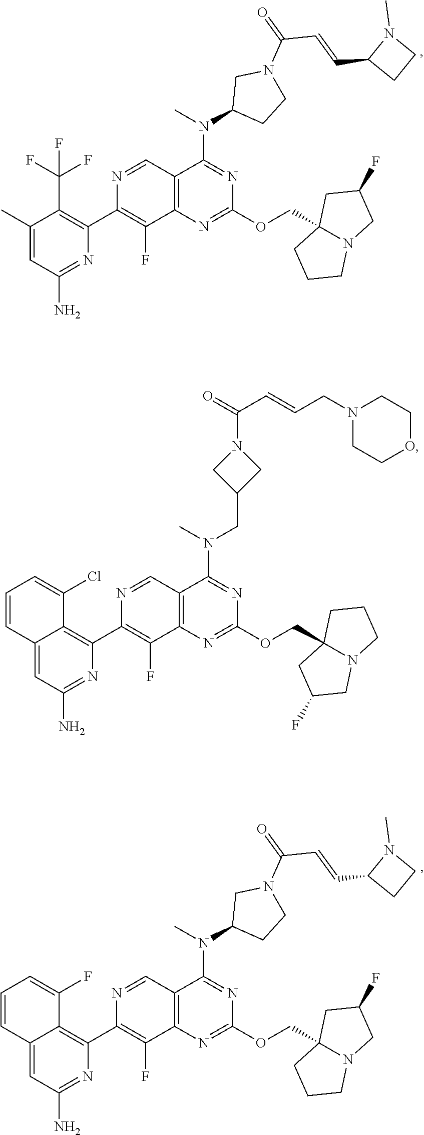 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F ; CN(CC1CN(C(=O)/C=C/CN2CCOCC2)C1)c1nc(OC[C@@]23CCCN2C[C@H](F)C3)nc2c(F)c(-c3nc(N)cc4cccc(Cl)c34)ncc12 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1]

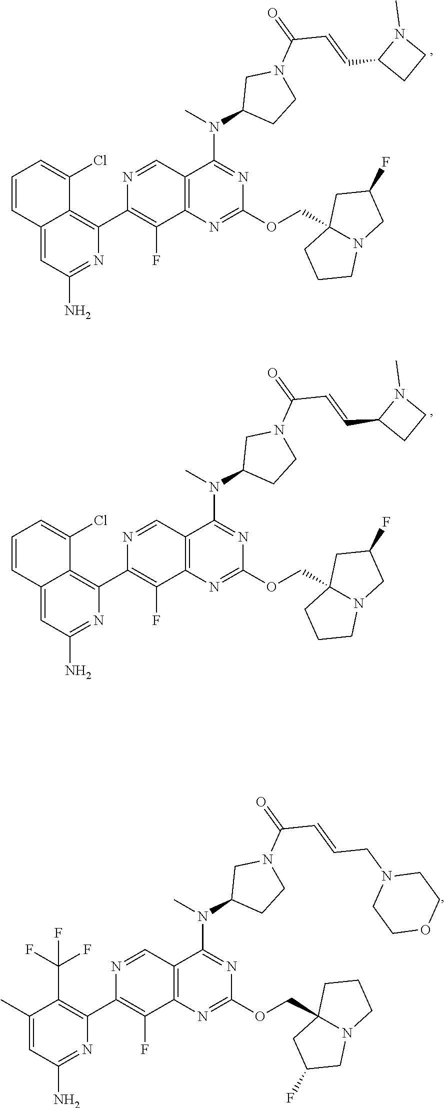 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F]

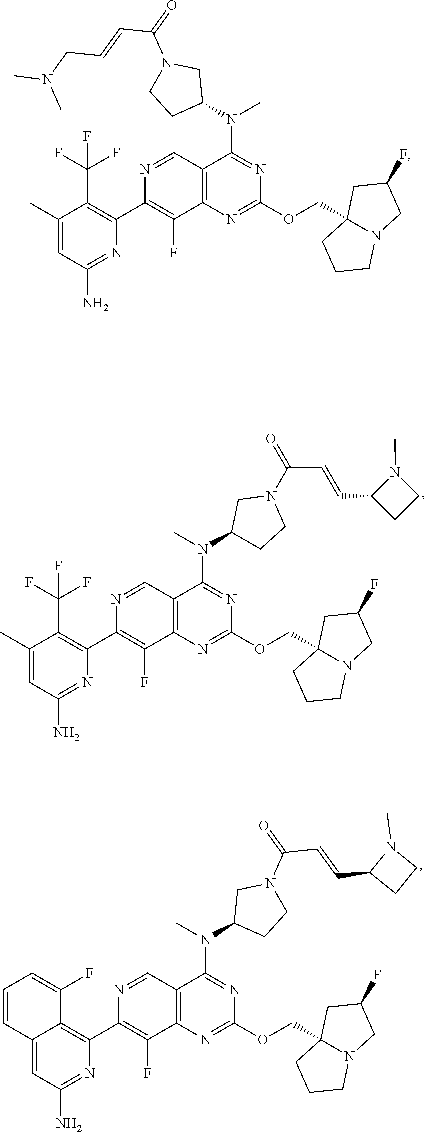 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1]

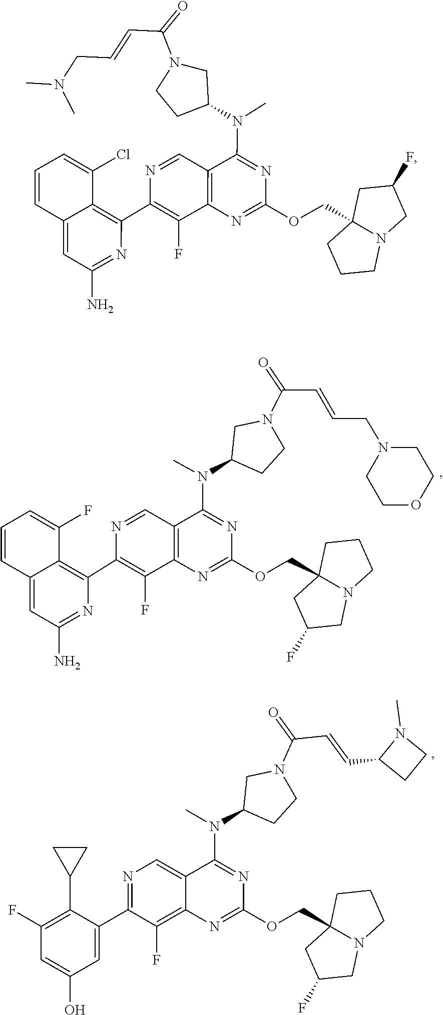 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1 ; CN1CC[C@@H]1/C=C/C(=O)N1CC[C@@H](N(C)c2nc(OC[C@@]34CCCN3C[C@H](F)C4)nc3c(F)c(-c4cc(O)cc(F)c4C4CC4)ncc23)C1]

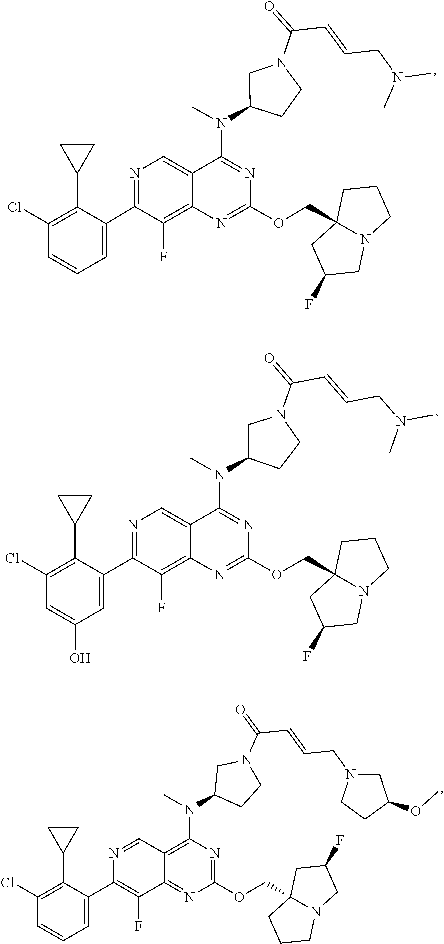 [SMILES: CN(C)C/C=C/C(=O)N1CC[C@@H](N(C)c2nc(OC[C@@]34CCCN3C[C@@H](F)C4)nc3c(F)c(-c4cccc(Cl)c4C4CC4)ncc23)C1 ; CN(C)C/C=C/C(=O)N1CC[C@@H](N(C)c2nc(OC[C@@]34CCCN3C[C@@H](F)C4)nc3c(F)c(-c4cc(O)cc(Cl)c4C4CC4)ncc23)C1 ; CO[C@H]1CCN(C/C=C/C(=O)N2CC[C@@H](N(C)c3nc(OC[C@@]45CCCN4C[C@H](F)C5)nc4c(F)c(-c5cccc(Cl)c5C5CC5)ncc34)C2)C1]

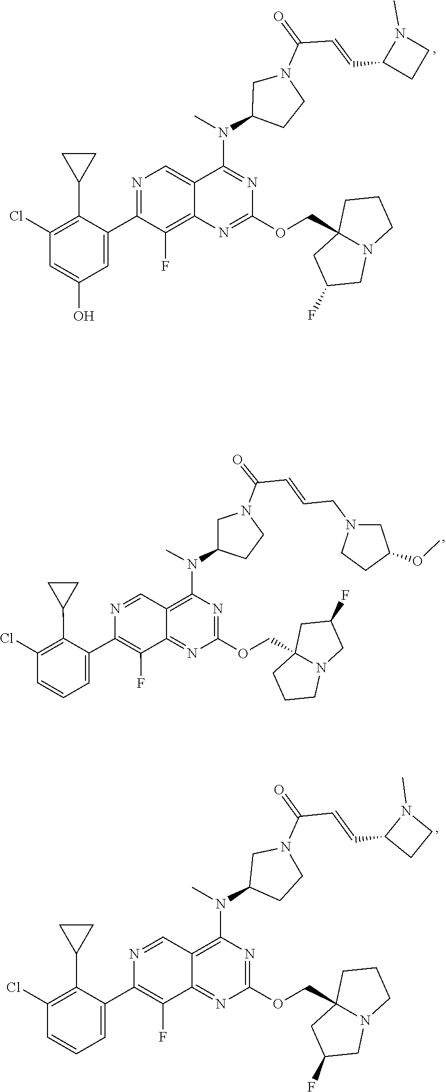 [SMILES: CN1CC[C@@H]1/C=C/C(=O)N1CC[C@@H](N(C)c2nc(OC[C@@]34CCCN3C[C@H](F)C4)nc3c(F)c(-c4cc(O)cc(Cl)c4C4CC4)ncc23)C1 ; CO[C@@H]1CCN(C/C=C/C(=O)N2CC[C@@H](N(C)c3nc(OC[C@@]45CCCN4C[C@H](F)C5)nc4c(F)c(-c5cccc(Cl)c5C5CC5)ncc34)C2)C1 ; CN1CC[C@@H]1/C=C/C(=O)N1CC[C@@H](N(C)c2nc(OC[C@@]34CCCN3C[C@@H](F)C4)nc3c(F)c(-c4cccc(Cl)c4C4CC4)ncc23)C1]

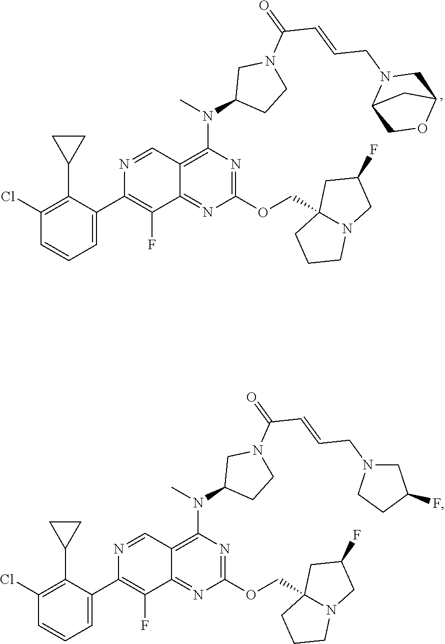 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@@H]3C[C@H]2CO3)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@H](F)C2)C1]

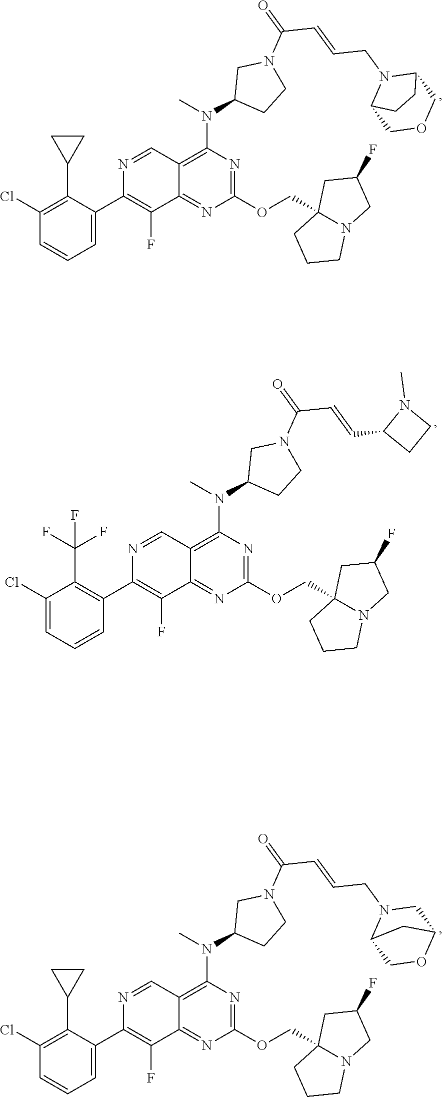 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2[C@@H]3CC[C@H]2COC3)C1 ; CN1CC[C@@H]1/C=C/C(=O)N1CC[C@@H](N(C)c2nc(OC[C@@]34CCCN3C[C@H](F)C4)nc3c(F)c(-c4cccc(Cl)c4C(F)(F)F)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@H]3C[C@@H]2CO3)C1]

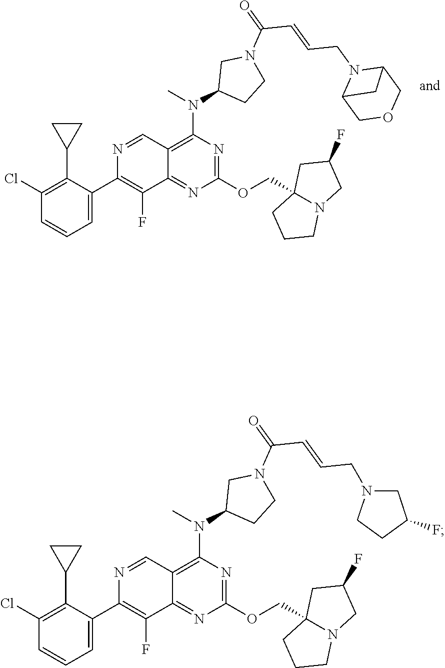 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C3COCC2C3)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@@H](F)C2)C1]

and all salts and isotopologues thereof.

Embodiment 310. The compound of embodiment 1, selected from the group consisting of:

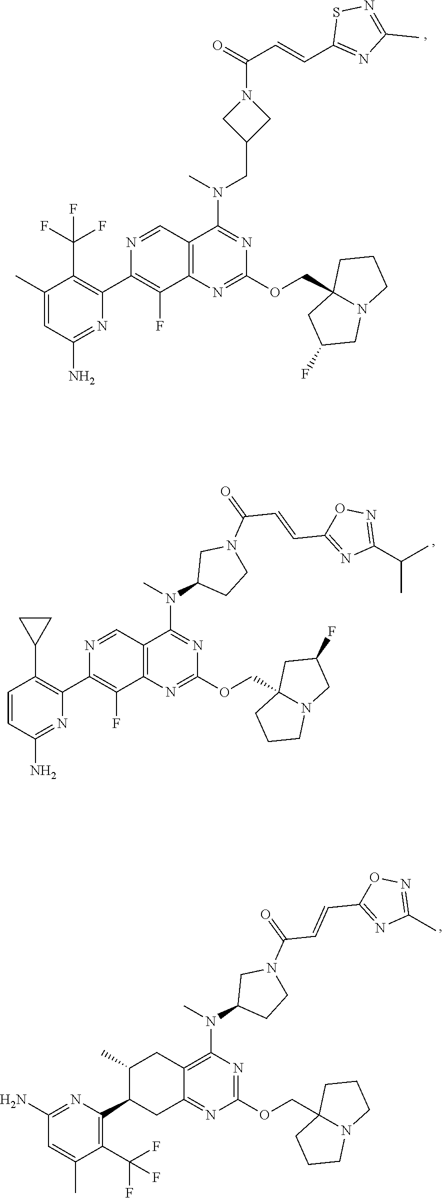

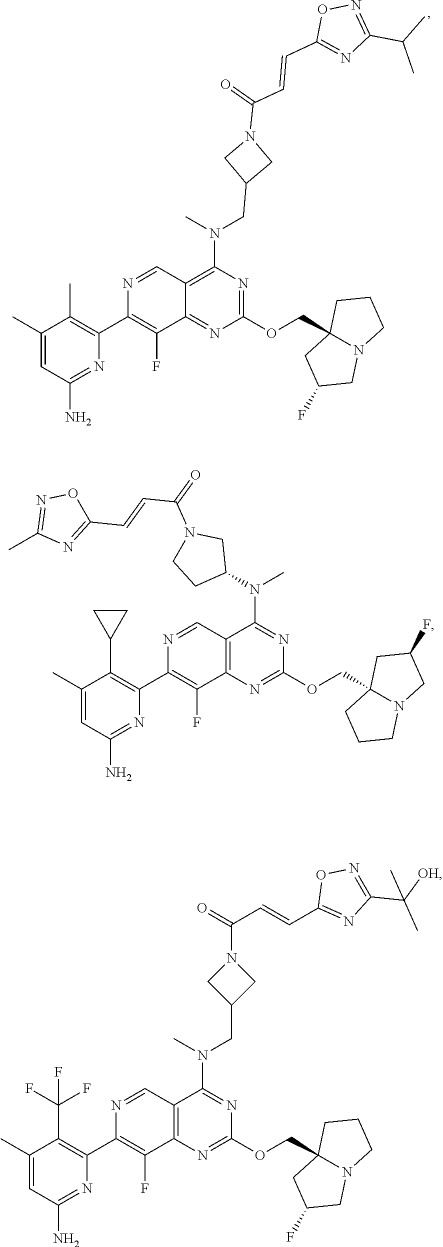

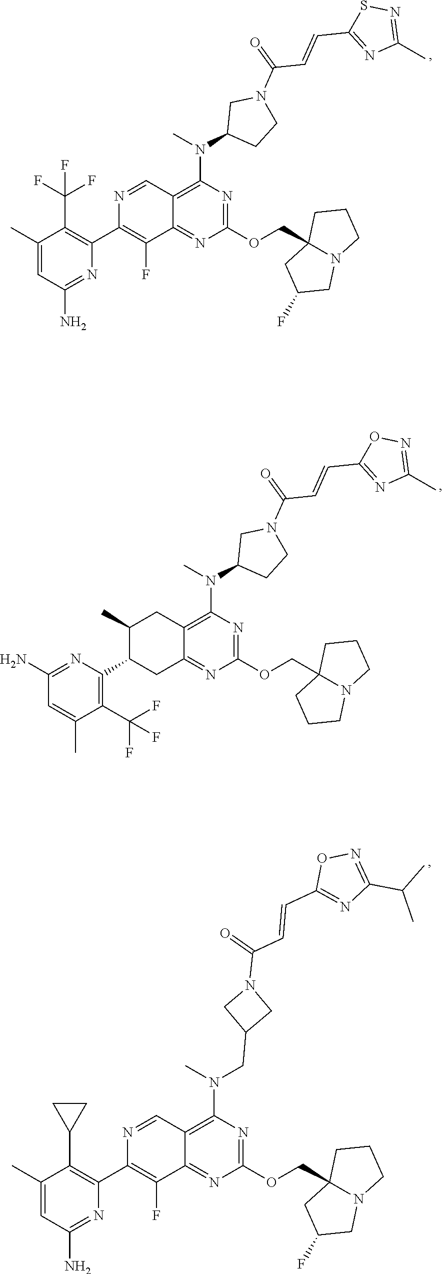

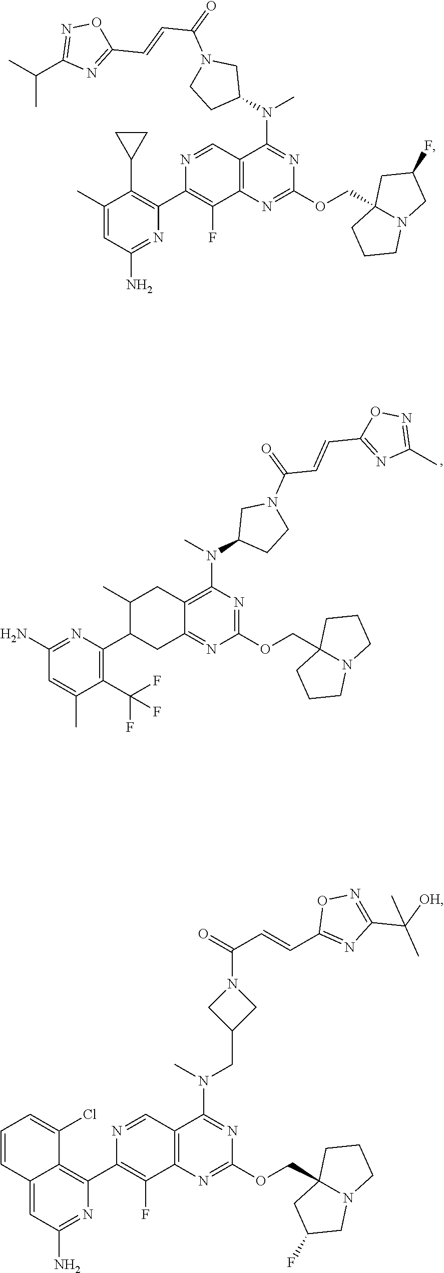 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1 ; Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12]

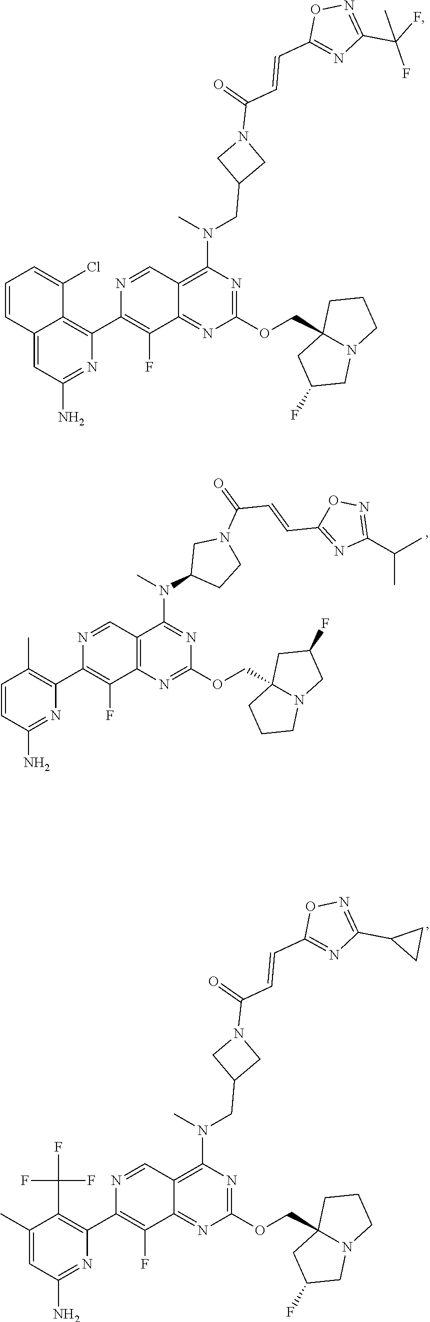

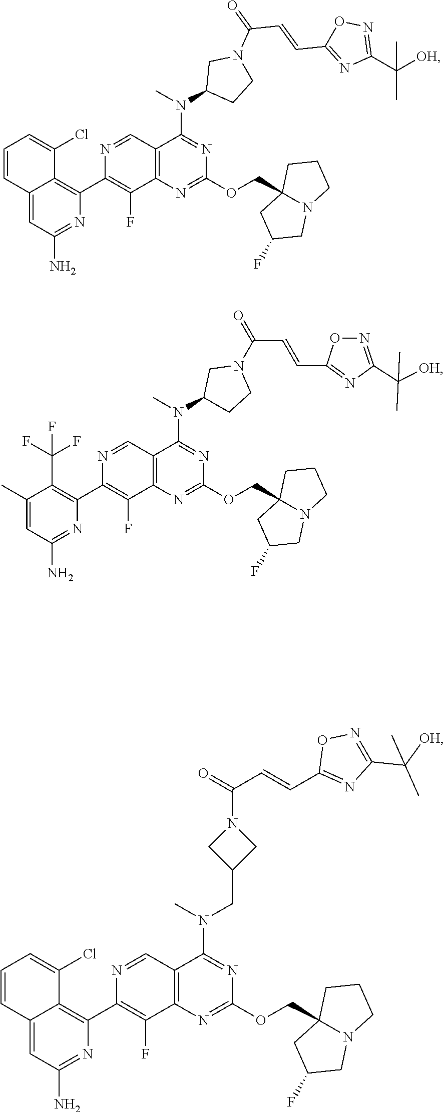 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12]

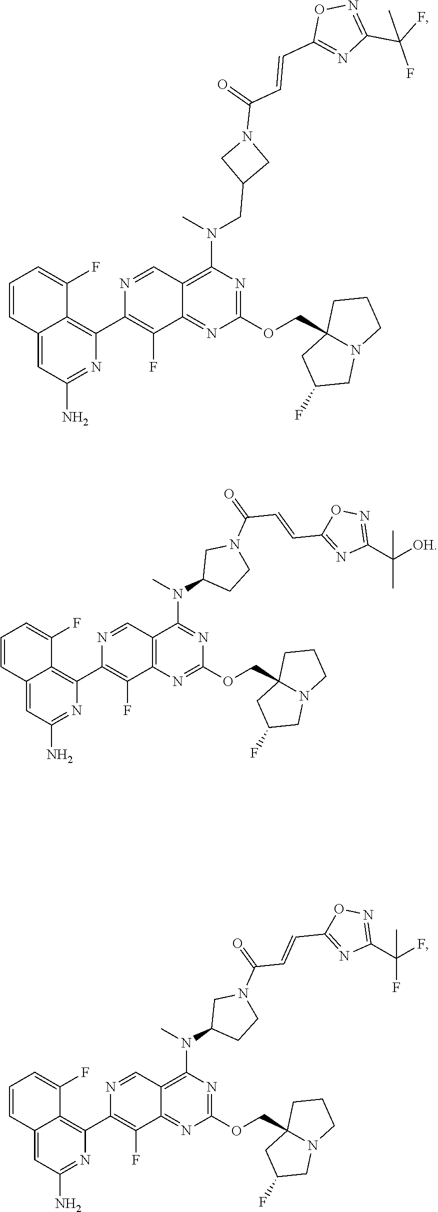 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1]

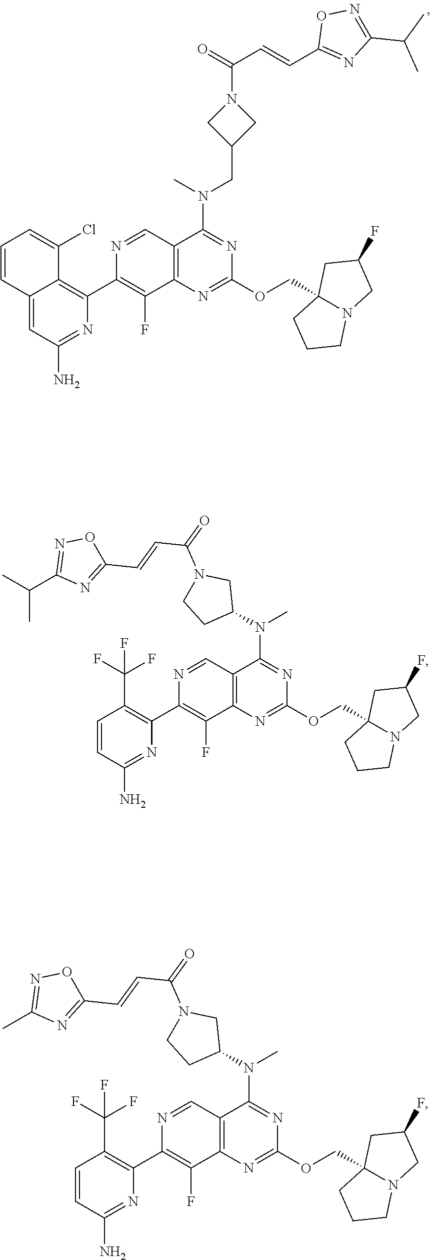

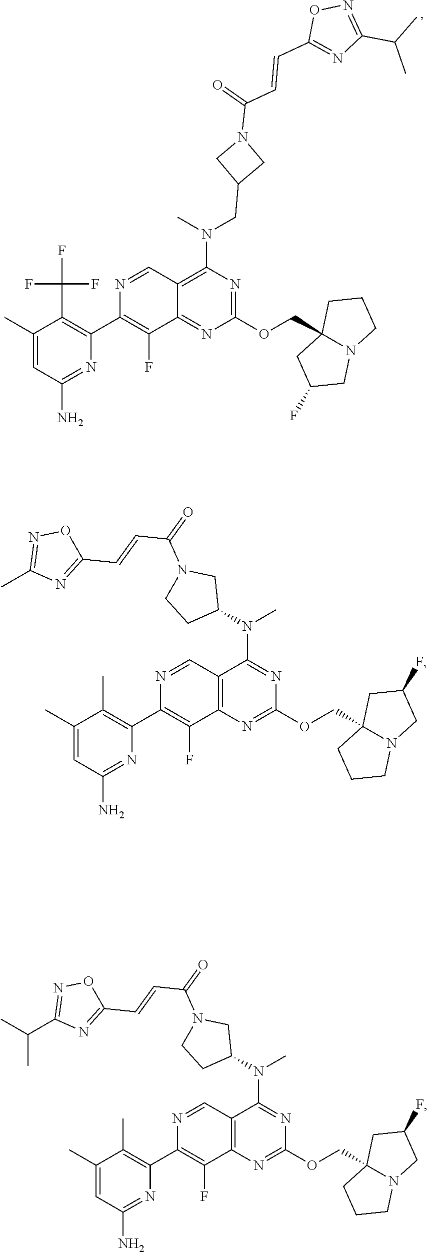

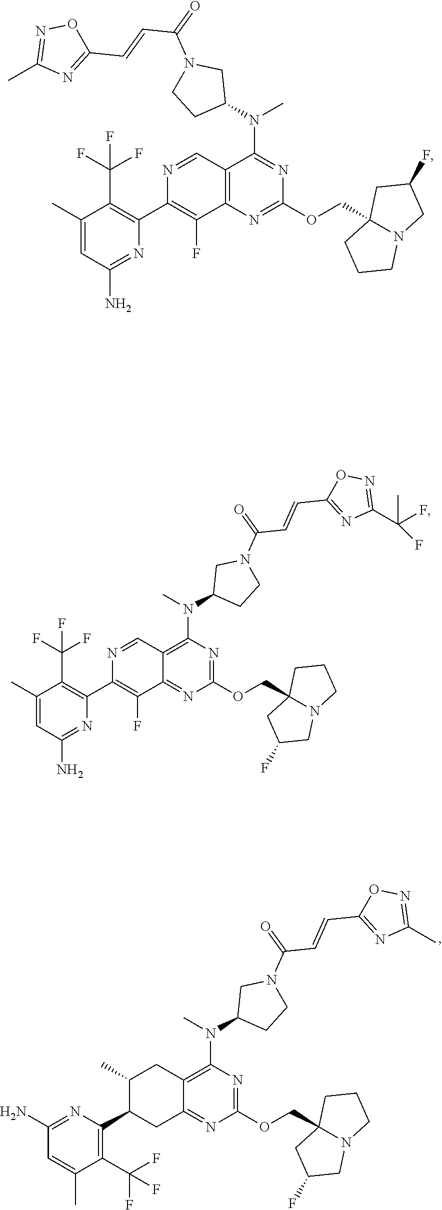 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1 ; Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1]

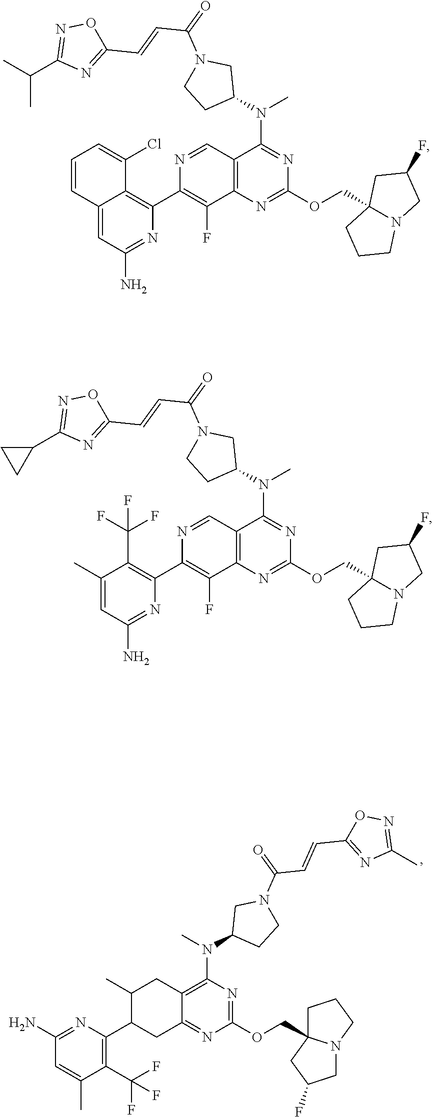 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1]

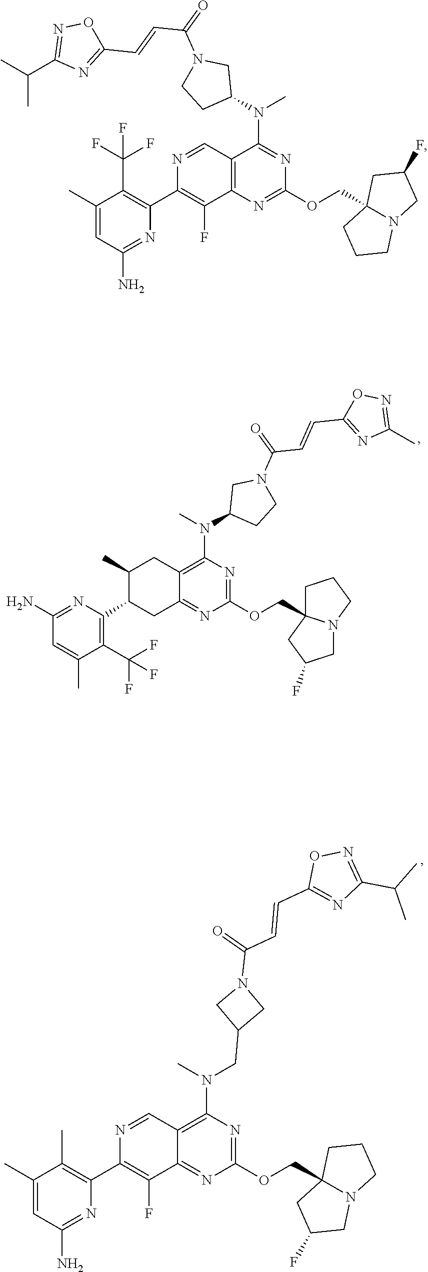

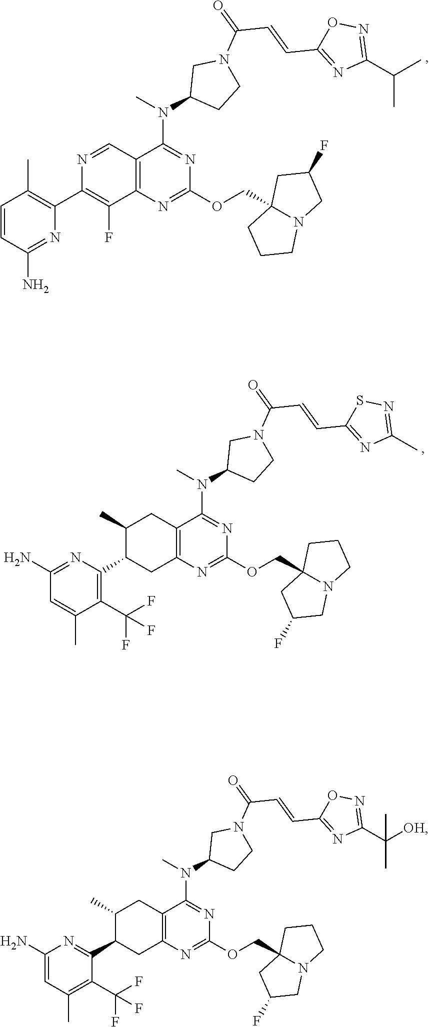 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F ; Cc1nsc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1 ; Cc1cc(N)nc([C@@H]2Cc3nc(OC[C@@]45CCCN4C[C@H](F)C5)nc(N(C)[C@@H]4CCN(C(=O)/C=C/c5nc(C(C)(C)O)no5)C4)c3C[C@H]2C)c1C(F)(F)F]

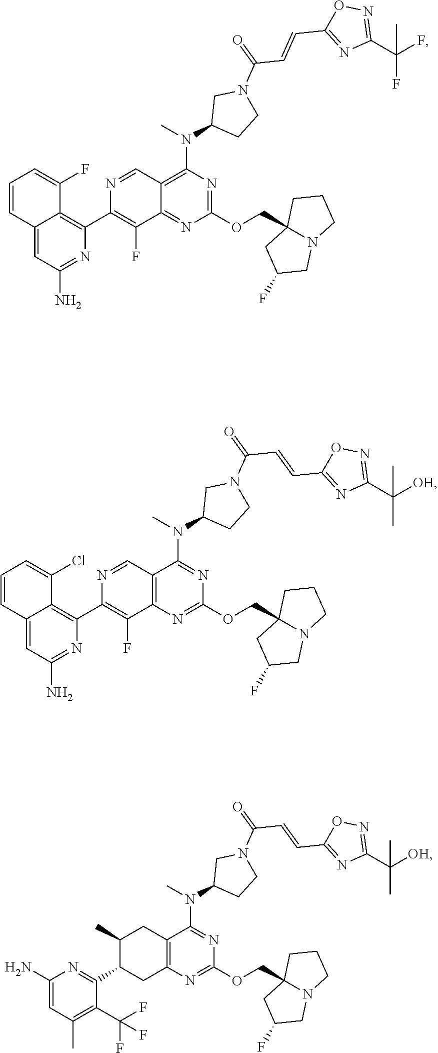 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1 ; CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc([C@H]2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F]

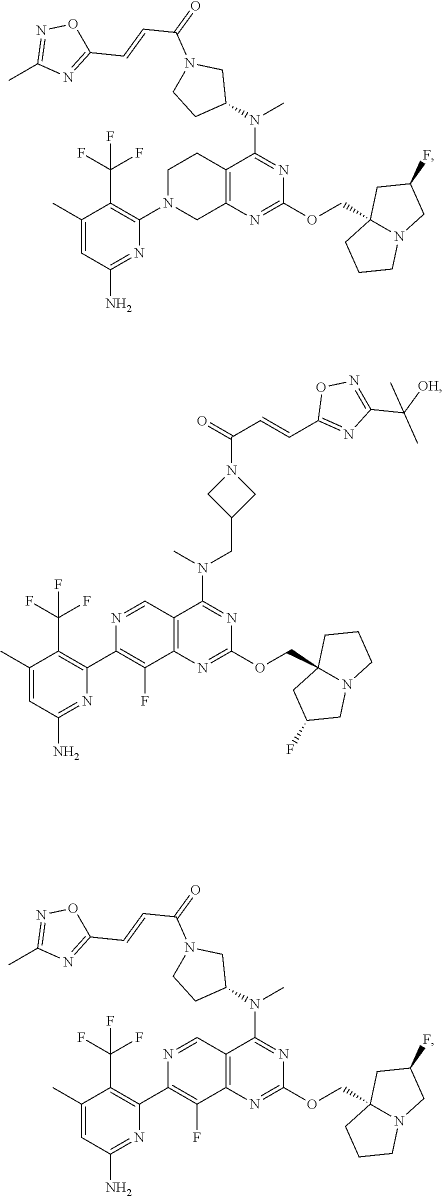 [SMILES: Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1 ; Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

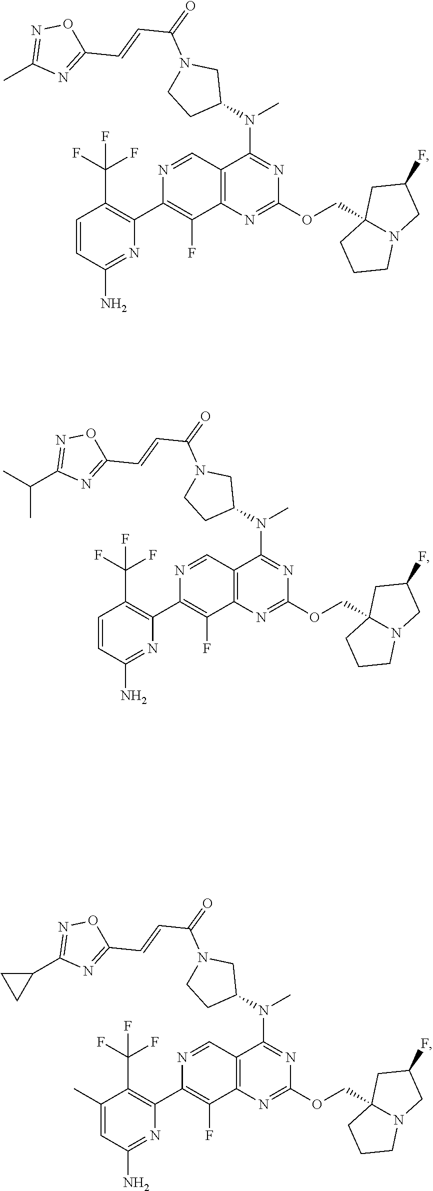 [SMILES: Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F]

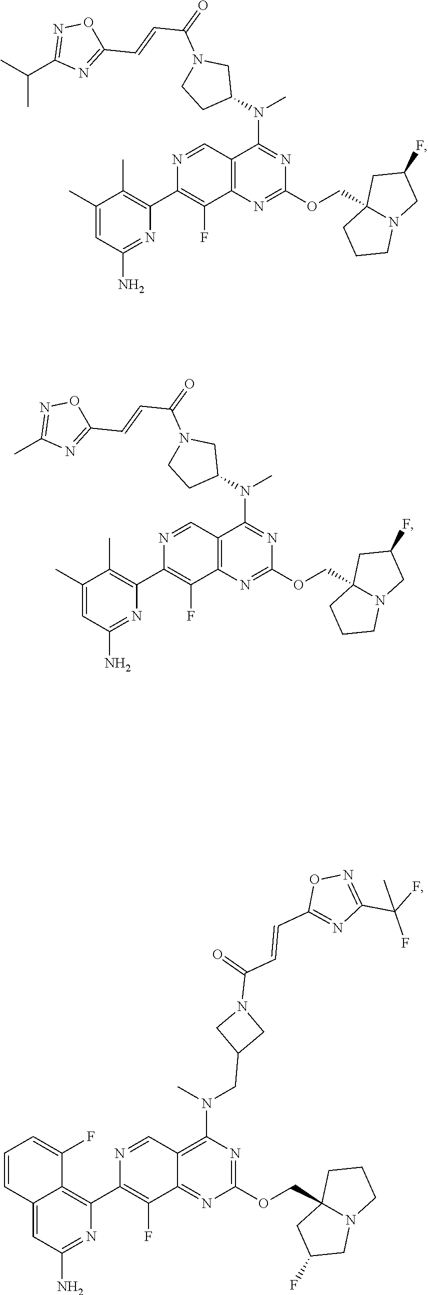 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C ; Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1 ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12]

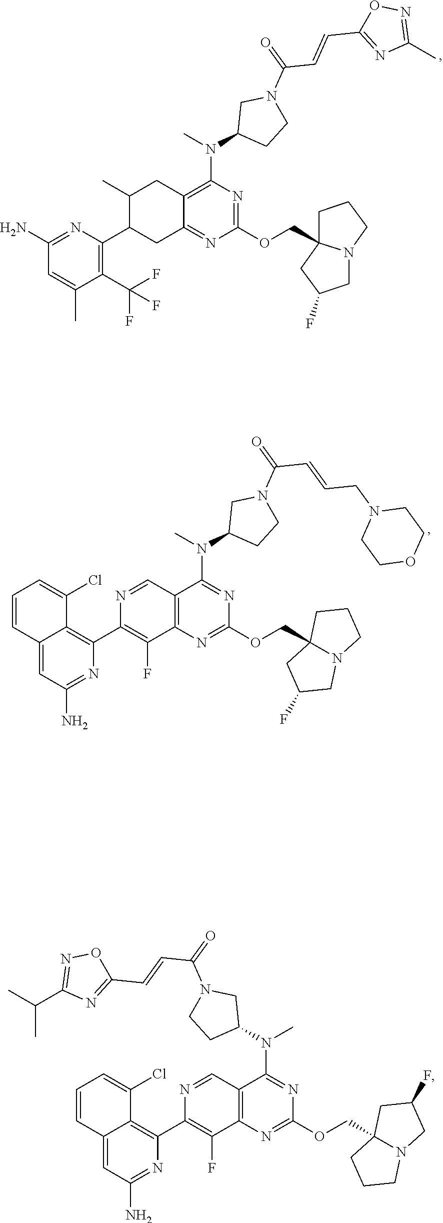

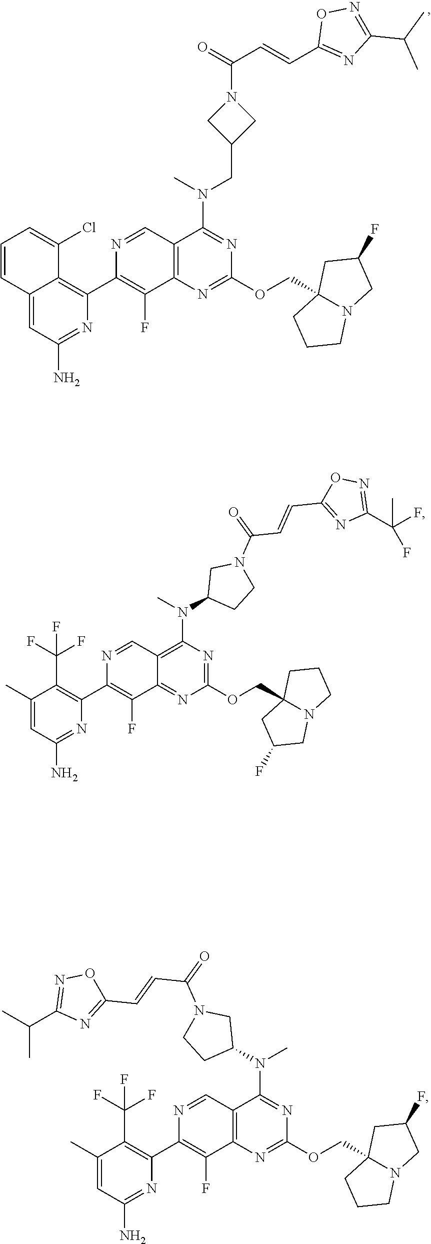

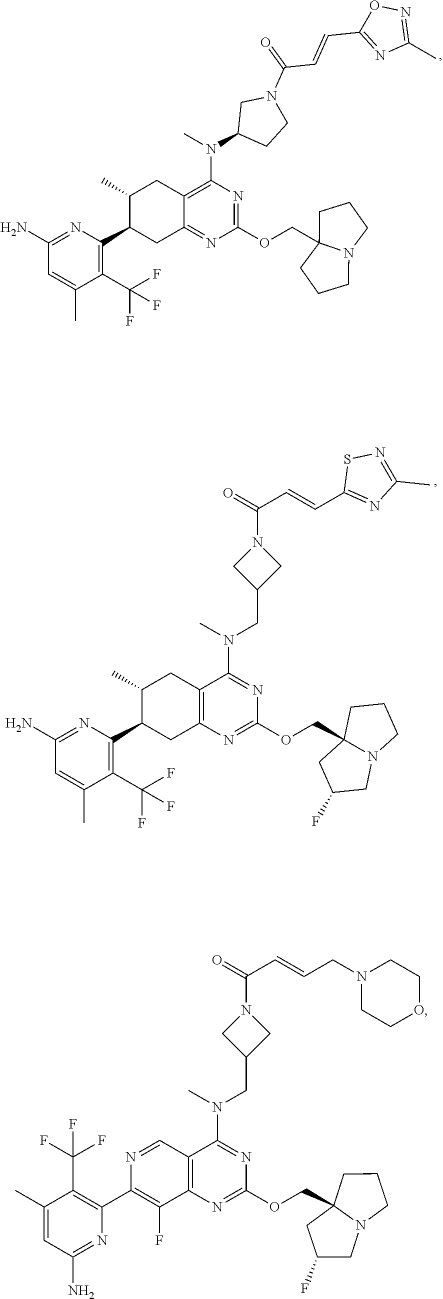

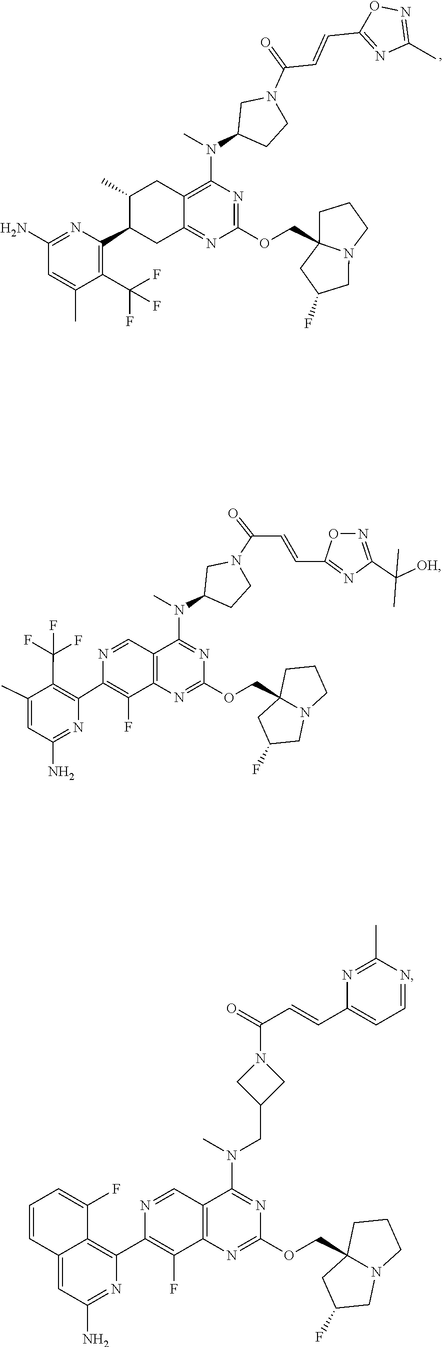

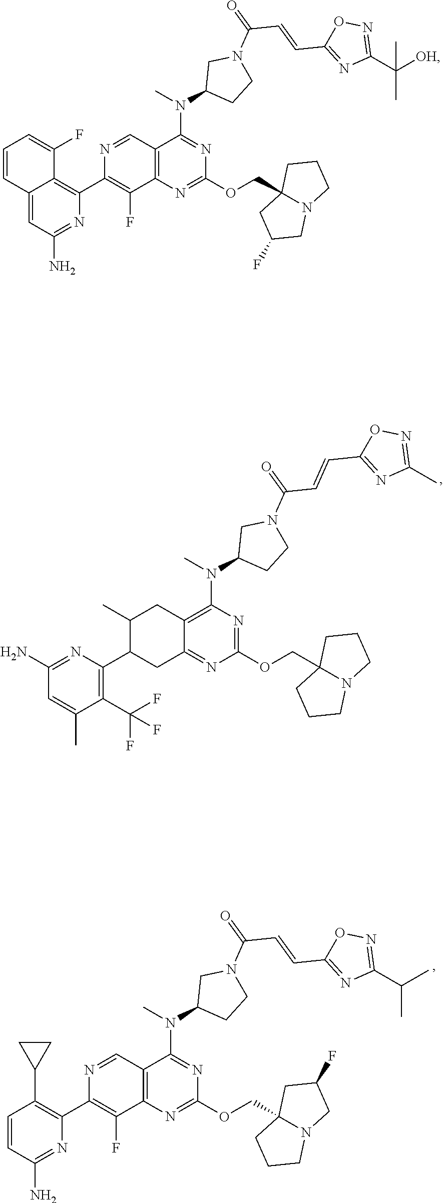 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1]

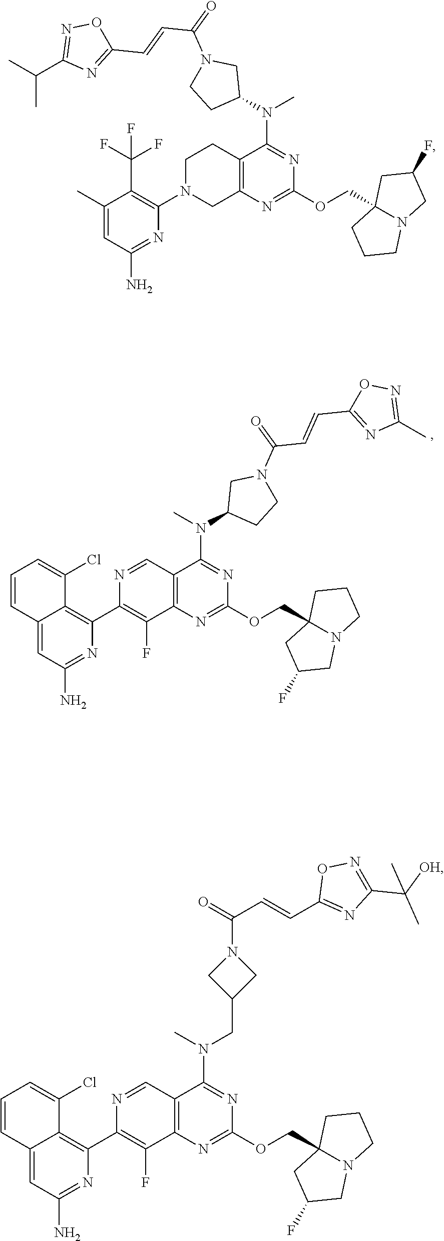 [SMILES: Cc1cc(N)nc(N2CCc3c(nc(OC[C@@]45CCCN4C[C@H](F)C5)nc3N(C)[C@@H]3CCN(C(=O)/C=C/c4nc(C(C)C)no4)C3)C2)c1C(F)(F)F ; Cc1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12]

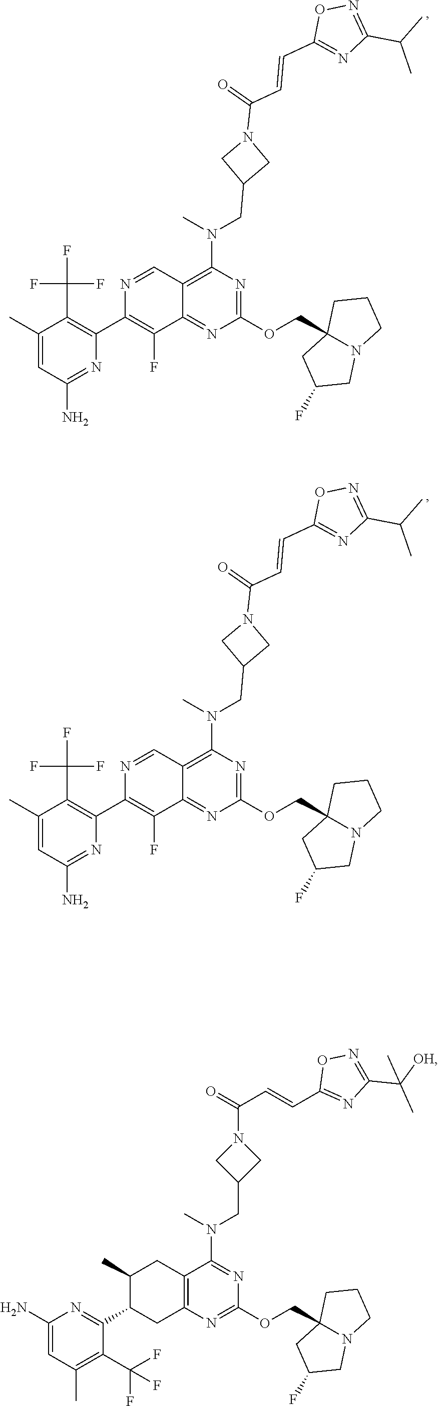

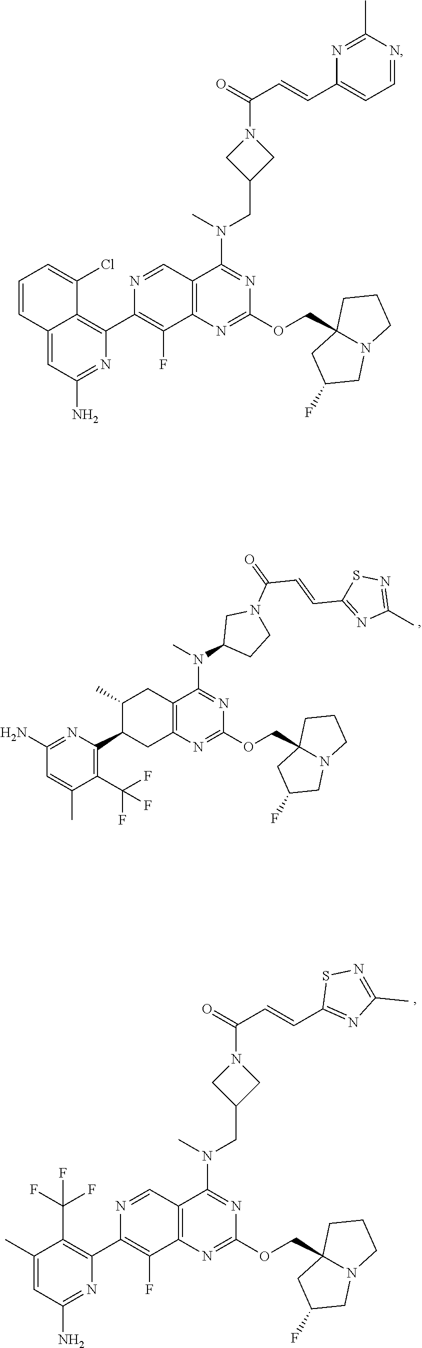

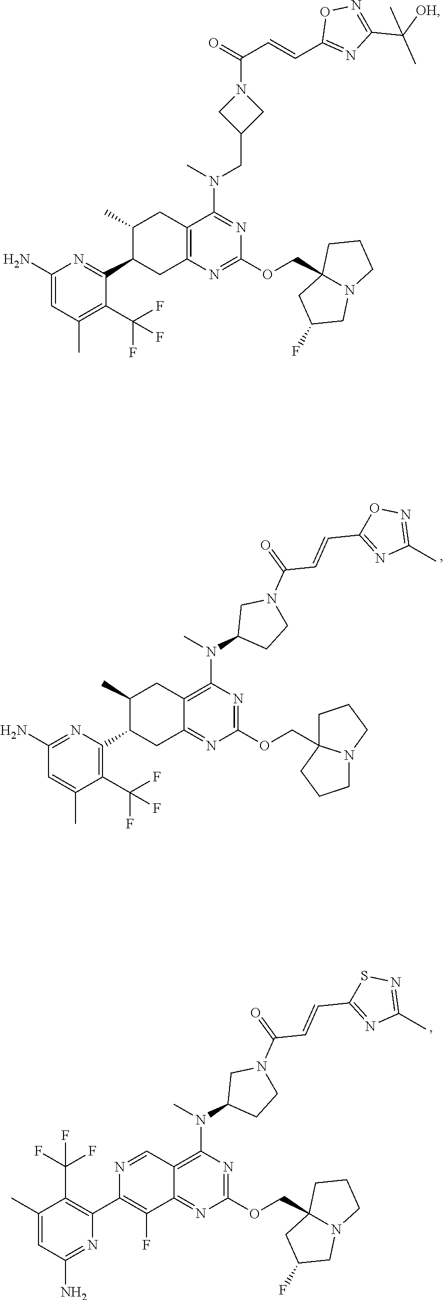

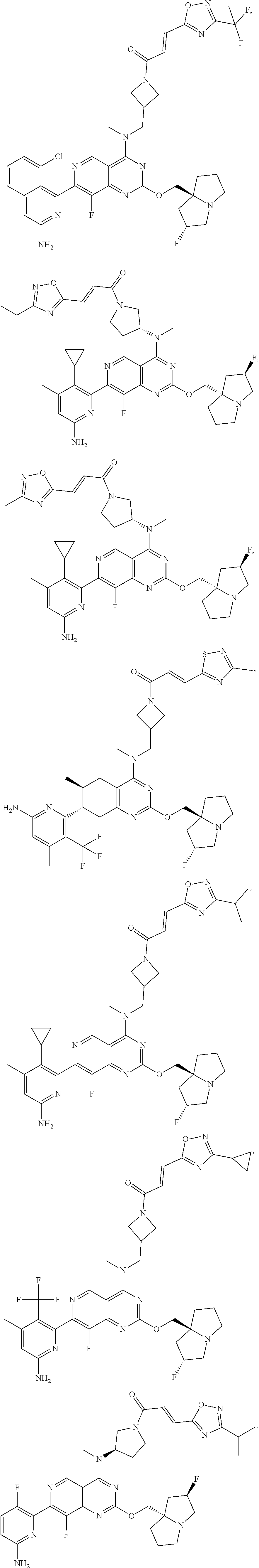

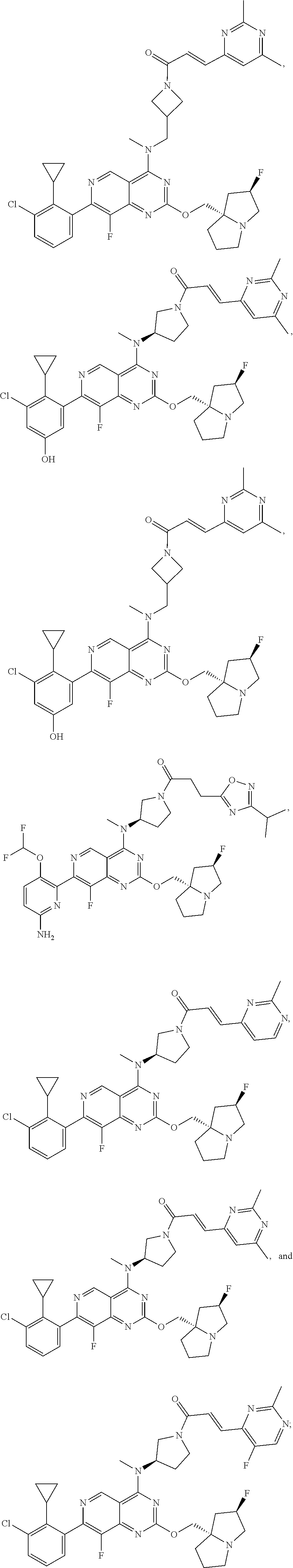

and all salts and isotopologues thereof.

Embodiment 311. The compound of embodiment 1, selected from the group consisting of:

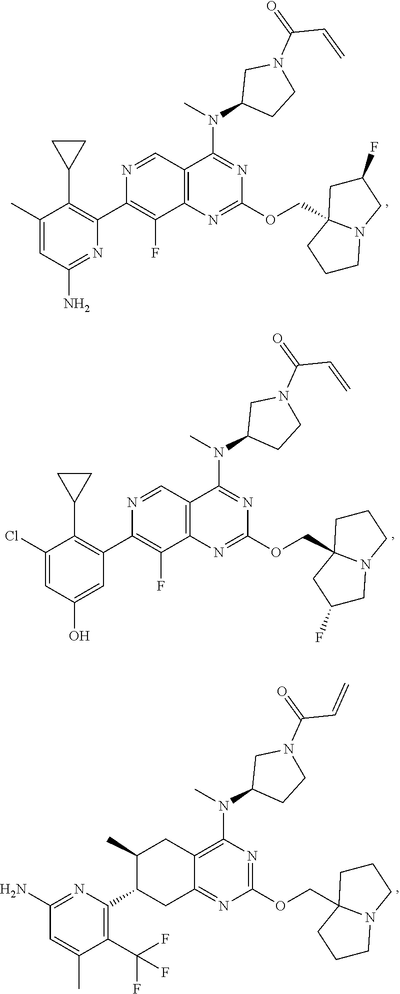 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1 ; C=CC(=O)N1CC[C@@H](N(C)c2nc(OC[C@@]34CCCN3C[C@H](F)C4)nc3c(F)c(-c4cc(O)cc(Cl)c4C4CC4)ncc23)C1 ; C=CC(=O)N1CC[C@@H](N(C)c2nc(OCC34CCCN3CCC4)nc3c2C[C@H](C)[C@@H](c2nc(N)cc(C)c2C(F)(F)F)C3)C1]

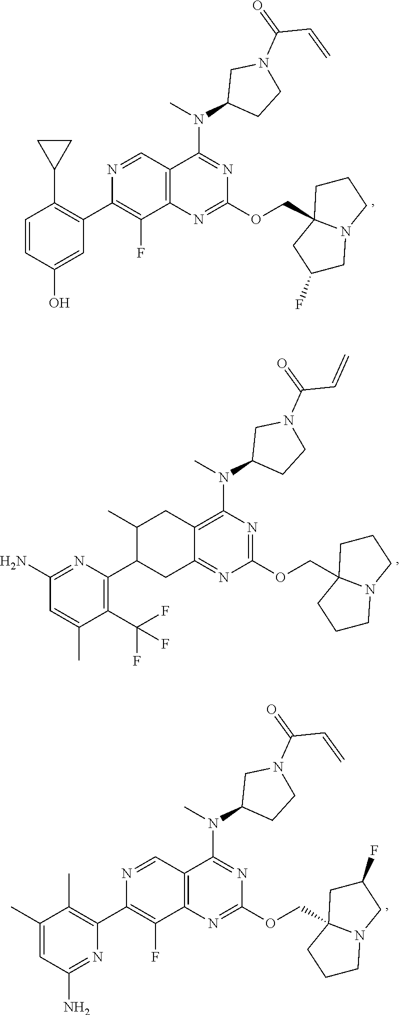 [SMILES: C=CC(=O)N1CC[C@@H](N(C)c2nc(OC[C@@]34CCCN3C[C@H](F)C4)nc3c(F)c(-c4cc(O)ccc4C4CC4)ncc23)C1 ; C=CC(=O)N1CC[C@@H](N(C)c2nc(OCC34CCCN3CCC4)nc3c2CC(C)C(c2nc(N)cc(C)c2C(F)(F)F)C3)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1]

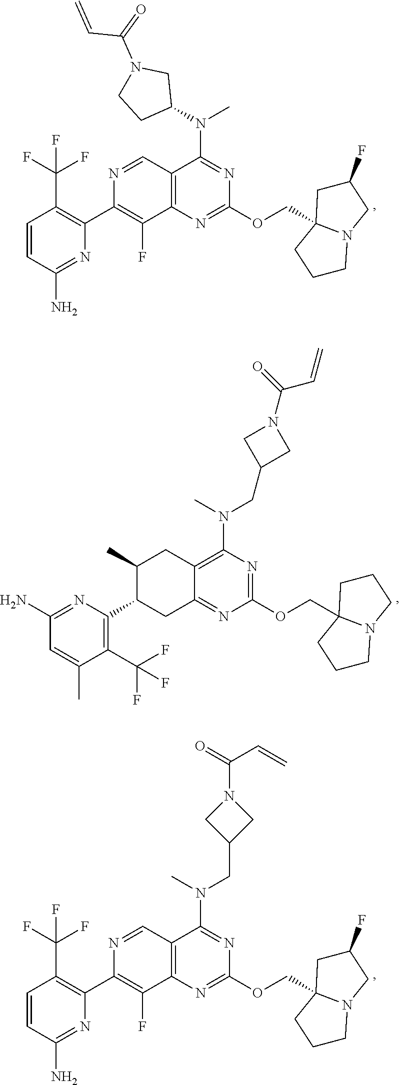 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1 ; C=CC(=O)N1CC(CN(C)c2nc(OCC34CCCN3CCC4)nc3c2C[C@H](C)[C@@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)ccc4C(F)(F)F)ncc23)C1]

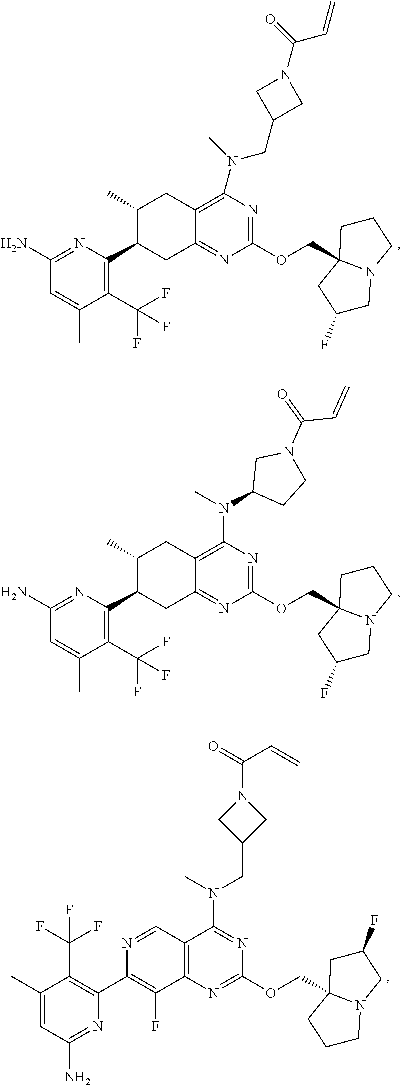 [SMILES: C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)cc(C)c4C(F)(F)F)ncc23)C1]

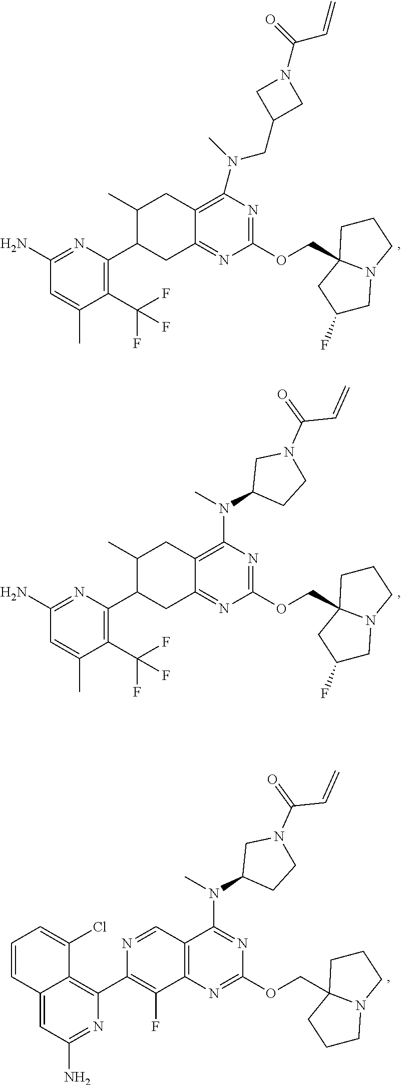 [SMILES: C=CC(=O)N1CC(CN(C)c2nc(OC[C@@]34CCCN3C[C@H](F)C4)nc3c2CC(C)C(c2nc(N)cc(C)c2C(F)(F)F)C3)C1 ; C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1 ; C=CC(=O)N1CC[C@@H](N(C)c2nc(OCC34CCCN3CCC4)nc3c(F)c(-c4nc(N)cc5cccc(Cl)c45)ncc23)C1]

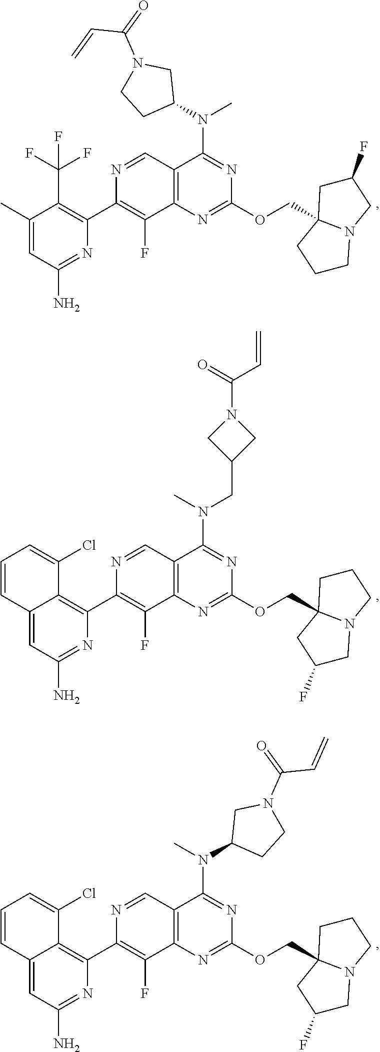 [SMILES: C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1 ; C=CC(=O)N1CC(CN(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OC[C@@]34CCCN3C[C@H](F)C4)nc3c(F)c(-c4nc(N)cc5cccc(Cl)c45)ncc23)C1]

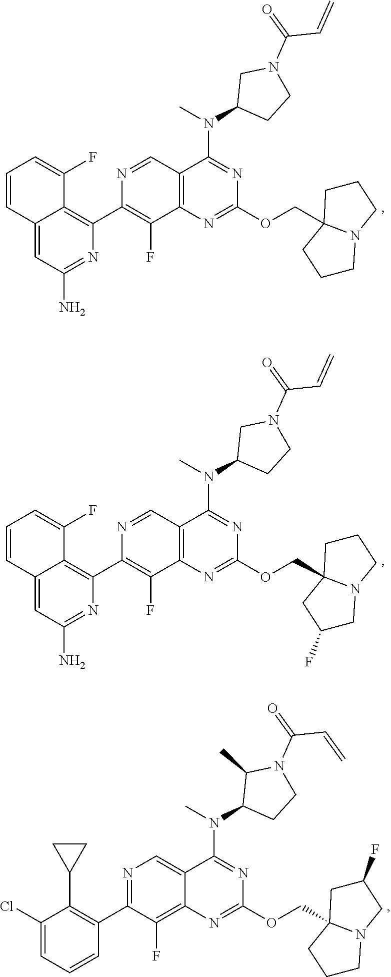 [SMILES: C=CC(=O)N1CC[C@@H](N(C)c2nc(OCC34CCCN3CCC4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C]

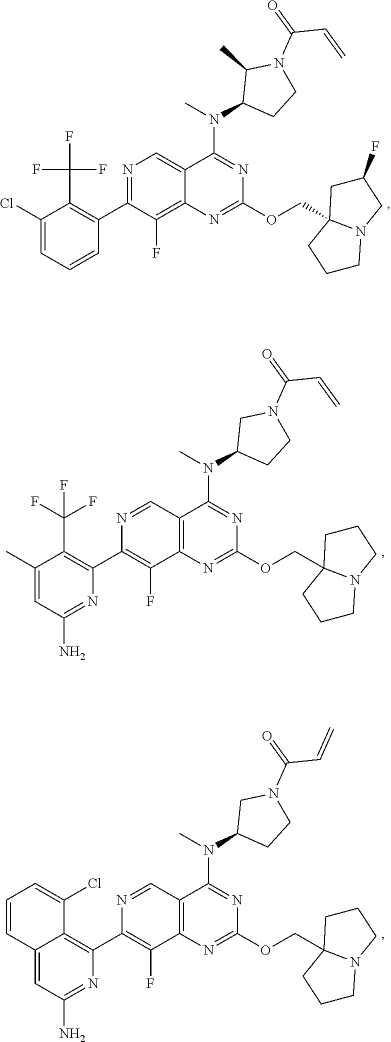 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OCC34CCCN3CCC4)nc3c(F)c(-c4nc(N)cc(C)c4C(F)(F)F)ncc23)C1 ; C=CC(=O)N1CC[C@@H](N(C)c2nc(OCC34CCCN3CCC4)nc3c(F)c(-c4nc(N)cc5cccc(Cl)c45)ncc23)C1]

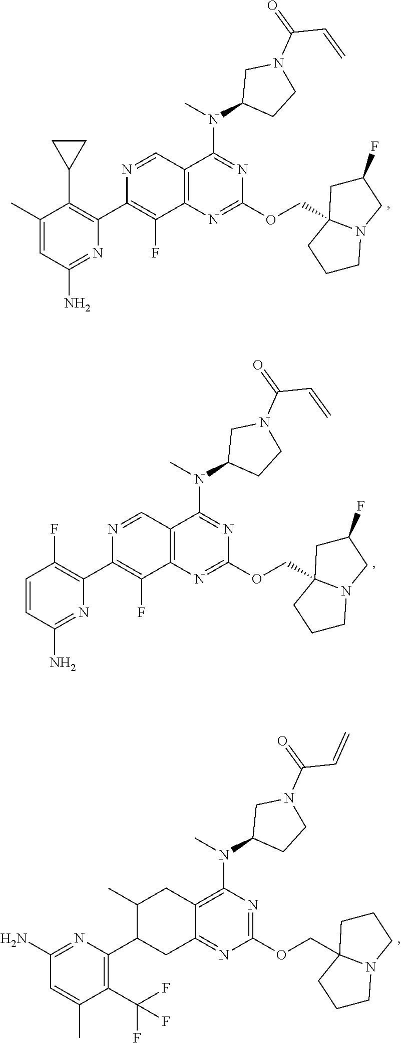 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1 ; C=CC(=O)N1CC[C@@H](N(C)c2nc(OC[C@@]34CCCN3C[C@H](F)C4)nc3c(F)c(-c4nc(N)ccc4F)ncc23)C1 ; C=CC(=O)N1CC[C@@H](N(C)c2nc(OCC34CCCN3CCC4)nc3c2CC(C)C(c2nc(N)cc(C)c2C(F)(F)F)C3)C1]

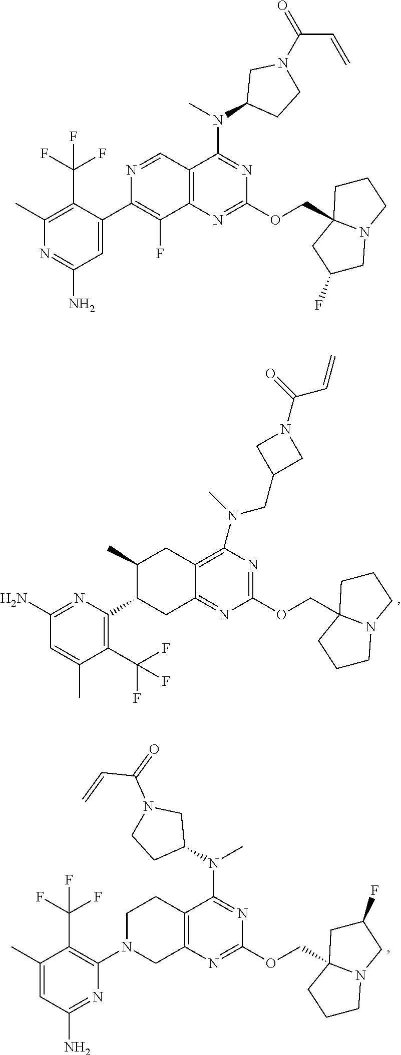 [SMILES: C=CC(=O)N1CC[C@@H](N(C)c2nc(OC[C@@]34CCCN3C[C@H](F)C4)nc3c(F)c(-c4cc(N)nc(C)c4C(F)(F)F)ncc23)C1 ; C=CC(=O)N1CC(CN(C)c2nc(OCC34CCCN3CCC4)nc3c2C[C@H](C)[C@@H](c2nc(N)cc(C)c2C(F)(F)F)C3)C1 ; C=CC(=O)N1CC[C@@H](N(C)c2nc(OC[C@@]34CCCN3C[C@H](F)C4)nc3c2CCN(c2nc(N)cc(C)c2C(F)(F)F)C3)C1]

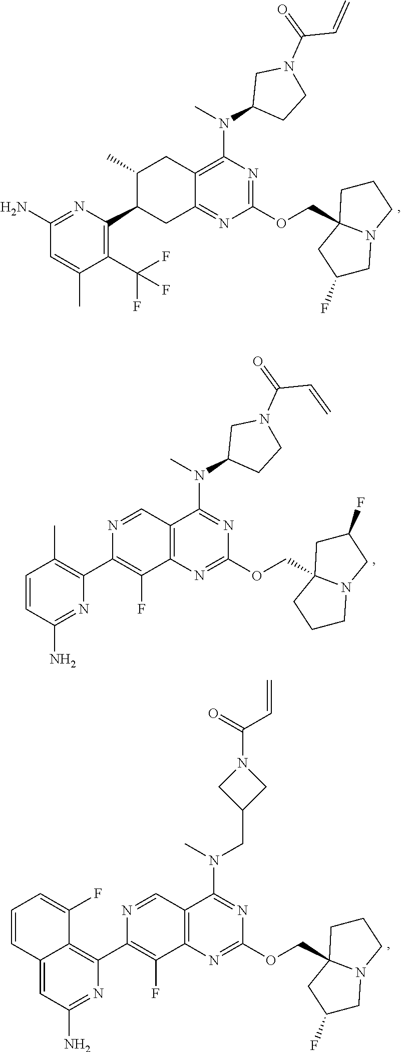 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC[C@@H](N(C)c2nc(OC[C@@]34CCCN3C[C@H](F)C4)nc3c(F)c(-c4nc(N)ccc4C)ncc23)C1 ; C=CC(=O)N1CC(CN(C)c2nc(OC[C@@]34CCCN3C[C@H](F)C4)nc3c(F)c(-c4nc(N)cc5cccc(F)c45)ncc23)C1]

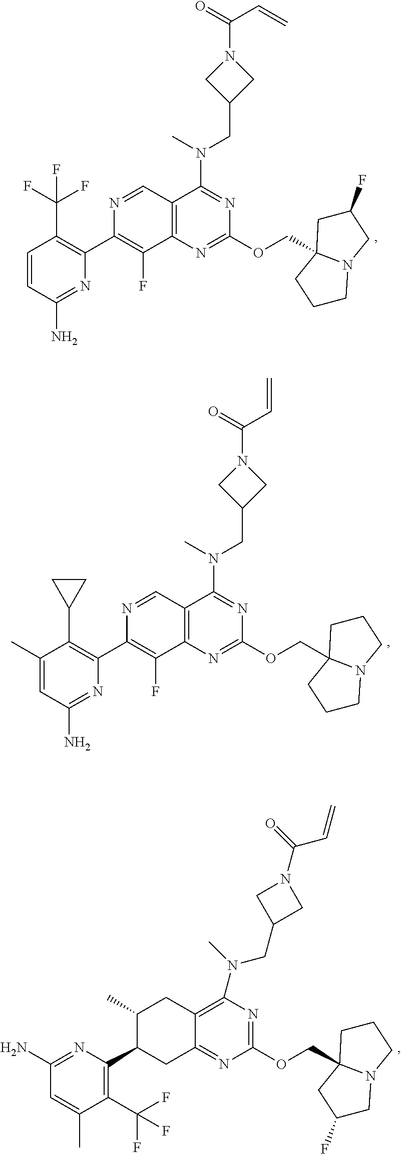 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1 ; C=CC(=O)N1CC(CN(C)c2nc(OCC34CCCN3CCC4)nc3c(F)c(-c4nc(N)cc(C)c4C4CC4)ncc23)C1 ; C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1]

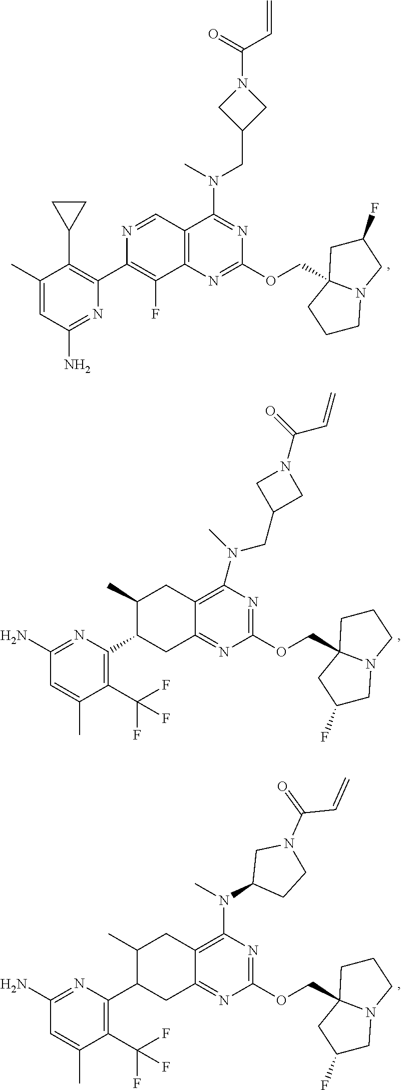 [SMILES: C=CC(=O)N1CC(CN(C)c2nc(OC[C@@]34CCCN3C[C@H](F)C4)nc3c(F)c(-c4nc(N)cc(C)c4C4CC4)ncc23)C1 ; C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1 ; C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1]

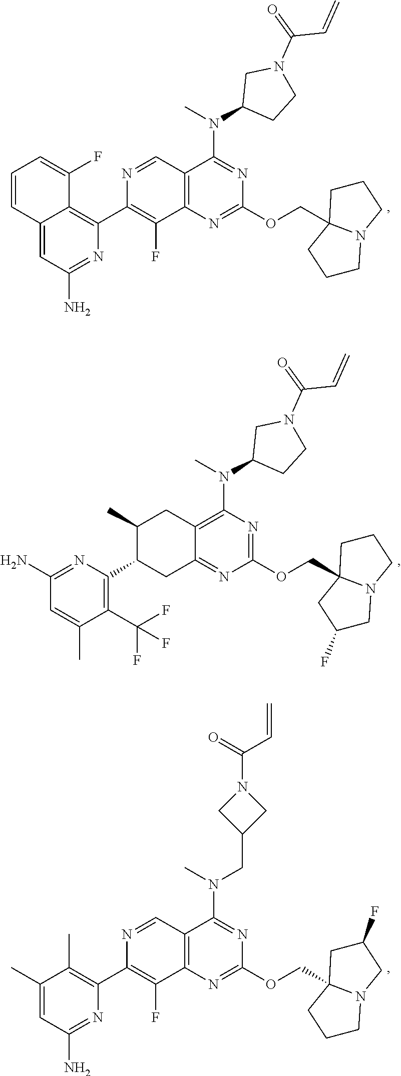 [SMILES: C=CC(=O)N1CC[C@@H](N(C)c2nc(OCC34CCCN3CCC4)nc3c(F)c(-c4nc(N)cc5cccc(F)c45)ncc23)C1 ; C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)cc(C)c4C)ncc23)C1]

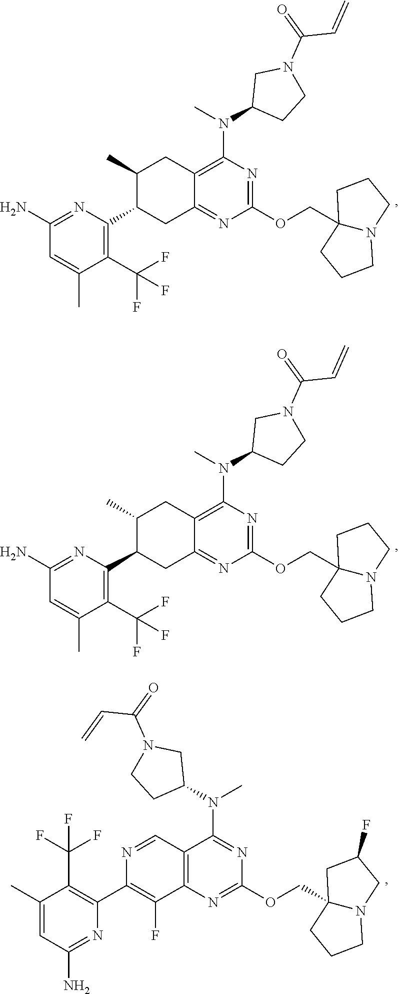

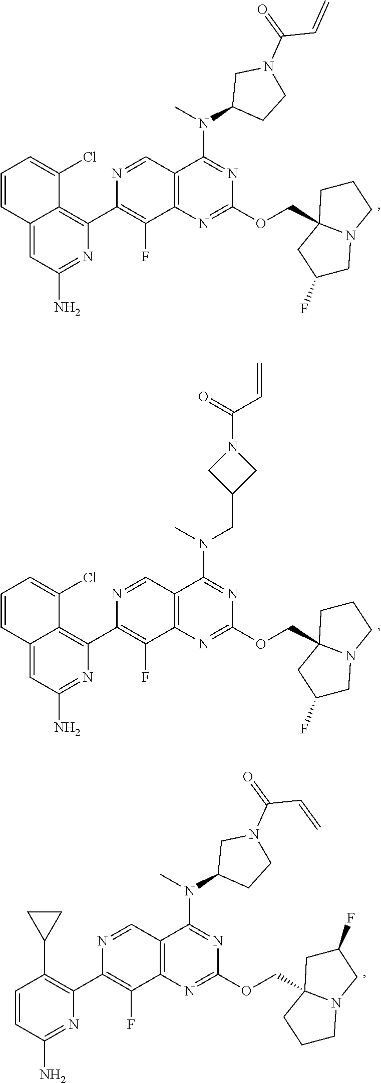 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1 ; C=CC(=O)N1CC(CN(C)c2nc(OC[C@@]34CCCN3C[C@H](F)C4)nc3c(F)c(-c4nc(N)cc5cccc(Cl)c45)ncc23)C1 ; C=CC(=O)N1CC[C@@H](N(C)c2nc(OC[C@@]34CCCN3C[C@H](F)C4)nc3c(F)c(-c4nc(N)ccc4C4CC4)ncc23)C1]

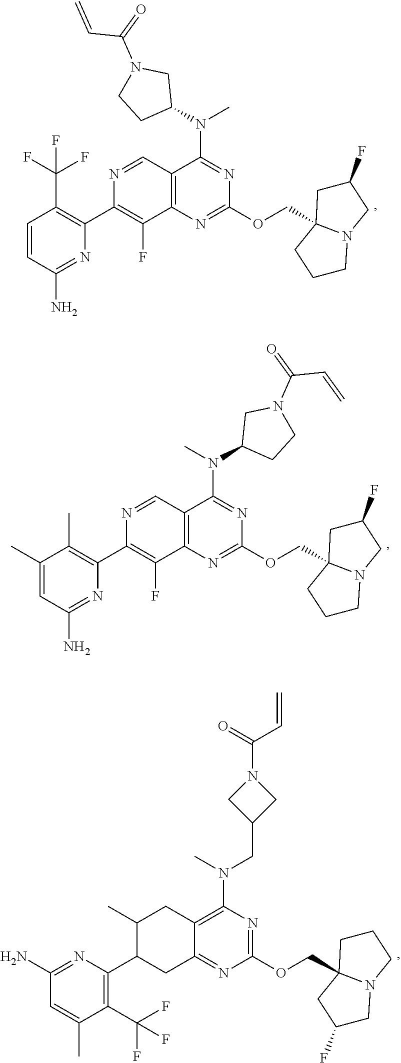 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1 ; C=CC(=O)N1CC(CN(C)c2nc(OC[C@@]34CCCN3C[C@H](F)C4)nc3c2CC(C)C(c2nc(N)cc(C)c2C(F)(F)F)C3)C1]

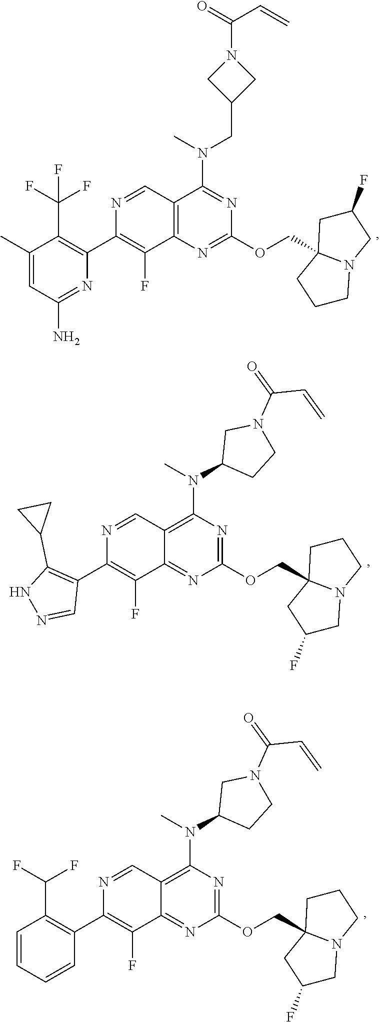 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1 ; C=CC(=O)N1CC[C@@H](N(C)c2nc(OC[C@@]34CCCN3C[C@H](F)C4)nc3c(F)c(-c4cn[nH]c4C4CC4)ncc23)C1 ; C=CC(=O)N1CC[C@@H](N(C)c2nc(OC[C@@]34CCCN3C[C@H](F)C4)nc3c(F)c(-c4ccccc4C(F)F)ncc23)C1]

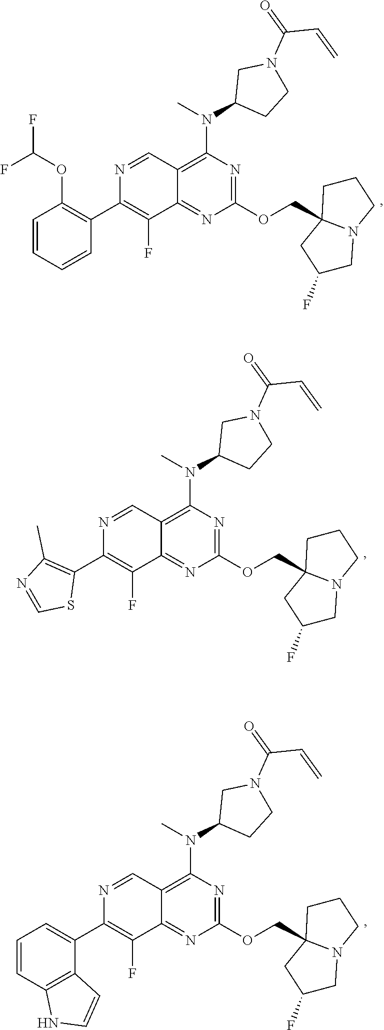 [SMILES: C=CC(=O)N1CC[C@@H](N(C)c2nc(OC[C@@]34CCCN3C[C@H](F)C4)nc3c(F)c(-c4ccccc4OC(F)F)ncc23)C1 ; C=CC(=O)N1CC[C@@H](N(C)c2nc(OC[C@@]34CCCN3C[C@H](F)C4)nc3c(F)c(-c4scnc4C)ncc23)C1 ; C=CC(=O)N1CC[C@@H](N(C)c2nc(OC[C@@]34CCCN3C[C@H](F)C4)nc3c(F)c(-c4cccc5[nH]ccc45)ncc23)C1]

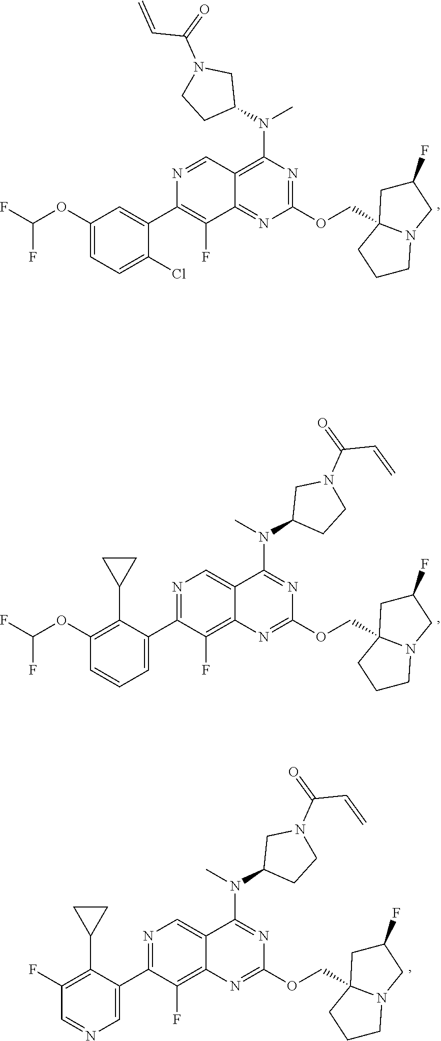 [SMILES: C=CC(=O)N1CC[C@@H](N(C)c2nc(OC[C@@]34CCCN3C[C@H](F)C4)nc3c(F)c(-c4cc(OC(F)F)ccc4Cl)ncc23)C1 ; C=CC(=O)N1CC[C@@H](N(C)c2nc(OC[C@@]34CCCN3C[C@H](F)C4)nc3c(F)c(-c4cccc(OC(F)F)c4C4CC4)ncc23)C1 ; C=CC(=O)N1CC[C@@H](N(C)c2nc(OC[C@@]34CCCN3C[C@H](F)C4)nc3c(F)c(-c4cncc(F)c4C4CC4)ncc23)C1]

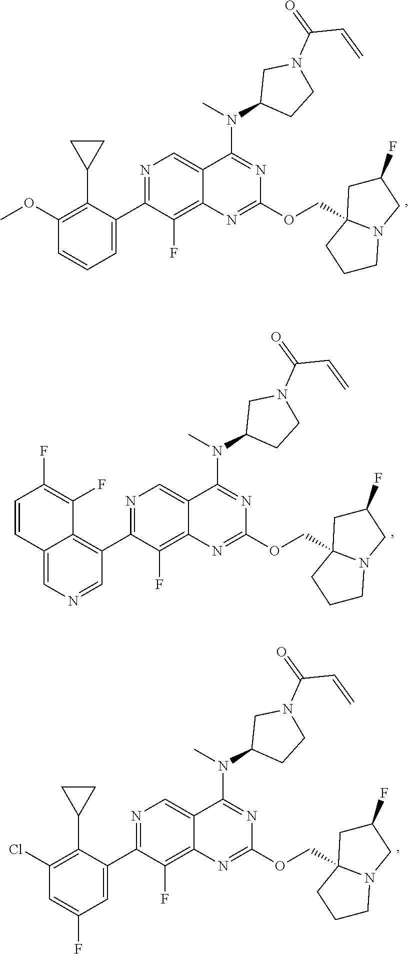 [SMILES: C=CC(=O)N1CC[C@@H](N(C)c2nc(OC[C@@]34CCCN3C[C@H](F)C4)nc3c(F)c(-c4cccc(OC)c4C4CC4)ncc23)C1 ; C=CC(=O)N1CC[C@@H](N(C)c2nc(OC[C@@]34CCCN3C[C@H](F)C4)nc3c(F)c(-c4cncc5ccc(F)c(F)c45)ncc23)C1 ; C=CC(=O)N1CC[C@@H](N(C)c2nc(OC[C@@]34CCCN3C[C@H](F)C4)nc3c(F)c(-c4cc(F)cc(Cl)c4C4CC4)ncc23)C1]

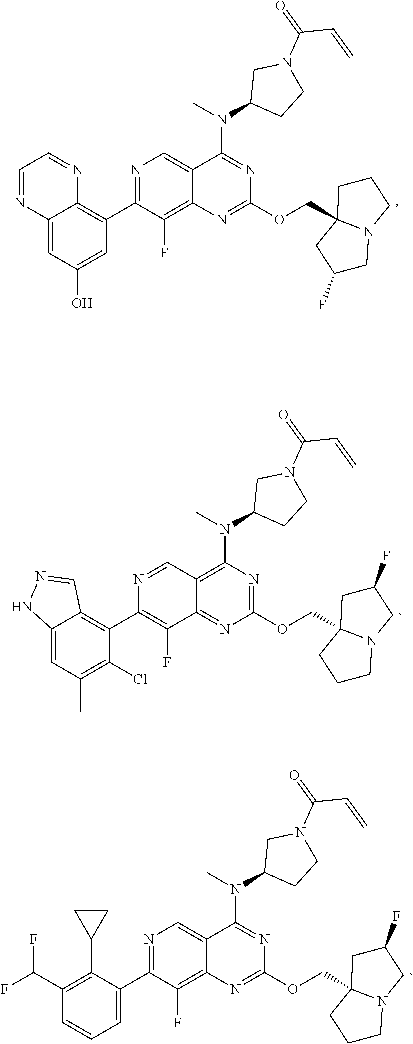 [SMILES: C=CC(=O)N1CC[C@@H](N(C)c2nc(OC[C@@]34CCCN3C[C@H](F)C4)nc3c(F)c(-c4cc(O)cc5nccnc45)ncc23)C1 ; C=CC(=O)N1CC[C@@H](N(C)c2nc(OC[C@@]34CCCN3C[C@H](F)C4)nc3c(F)c(-c4c(Cl)c(C)cc5[nH]ncc45)ncc23)C1 ; C=CC(=O)N1CC[C@@H](N(C)c2nc(OC[C@@]34CCCN3C[C@H](F)C4)nc3c(F)c(-c4cccc(C(F)F)c4C4CC4)ncc23)C1]

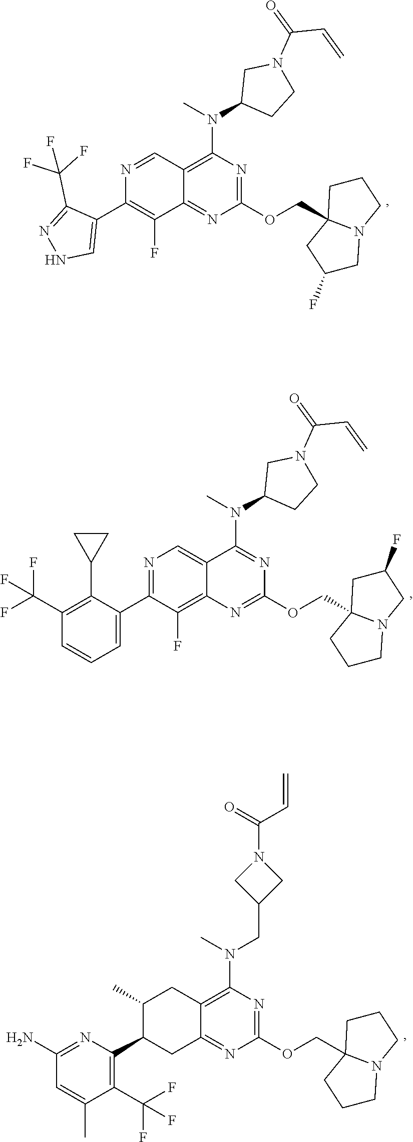 [SMILES: C=CC(=O)N1CC[C@@H](N(C)c2nc(OC[C@@]34CCCN3C[C@H](F)C4)nc3c(F)c(-c4c[nH]nc4C(F)(F)F)ncc23)C1 ; C=CC(=O)N1CC[C@@H](N(C)c2nc(OC[C@@]34CCCN3C[C@H](F)C4)nc3c(F)c(-c4cccc(C(F)(F)F)c4C4CC4)ncc23)C1 ; C=CC(=O)N1CC(CN(C)c2nc(OCC34CCCN3CCC4)nc3c2C[C@@H](C)[C@H](c2nc(N)cc(C)c2C(F)(F)F)C3)C1]

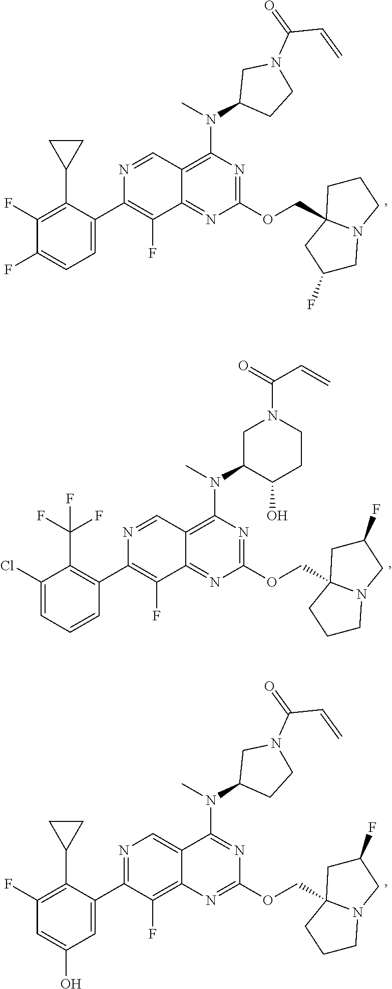 [SMILES: C=CC(=O)N1CC[C@@H](N(C)c2nc(OC[C@@]34CCCN3C[C@H](F)C4)nc3c(F)c(-c4ccc(F)c(F)c4C4CC4)ncc23)C1 ; C=CC(=O)N1CC[C@H](O)[C@@H](N(C)c2nc(OC[C@@]34CCCN3C[C@H](F)C4)nc3c(F)c(-c4cccc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(O)cc(F)c4C4CC4)ncc23)C1]

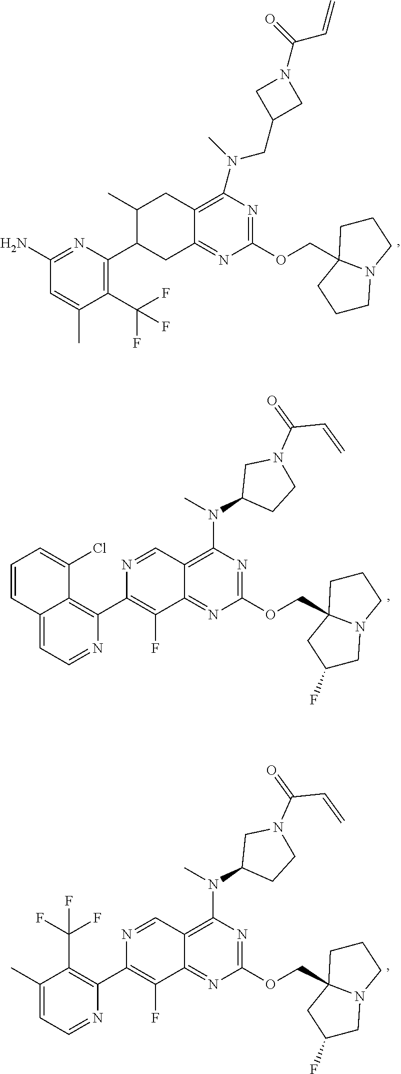 [SMILES: C=CC(=O)N1CC(CN(C)c2nc(OCC34CCCN3CCC4)nc3c2CC(C)C(c2nc(N)cc(C)c2C(F)(F)F)C3)C1 ; C=CC(=O)N1CC[C@@H](N(C)c2nc(OC[C@@]34CCCN3C[C@H](F)C4)nc3c(F)c(-c4nccc5cccc(Cl)c45)ncc23)C1 ; C=CC(=O)N1CC[C@@H](N(C)c2nc(OC[C@@]34CCCN3C[C@H](F)C4)nc3c(F)c(-c4nccc(C)c4C(F)(F)F)ncc23)C1]

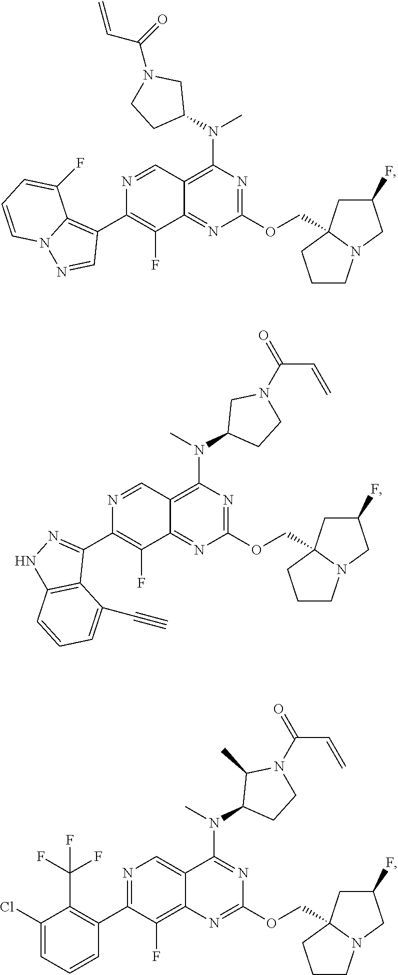 [SMILES: C=CC(=O)N1CC[C@@H](N(C)c2nc(OC[C@@]34CCCN3C[C@H](F)C4)nc3c(F)c(-c4cnn5cccc(F)c45)ncc23)C1 ; C#Cc1cccc2[nH]nc(-c3ncc4c(N(C)[C@@H]5CCN(C(=O)C=C)C5)nc(OC[C@@]56CCCN5C[C@H](F)C6)nc4c3F)c12 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C]

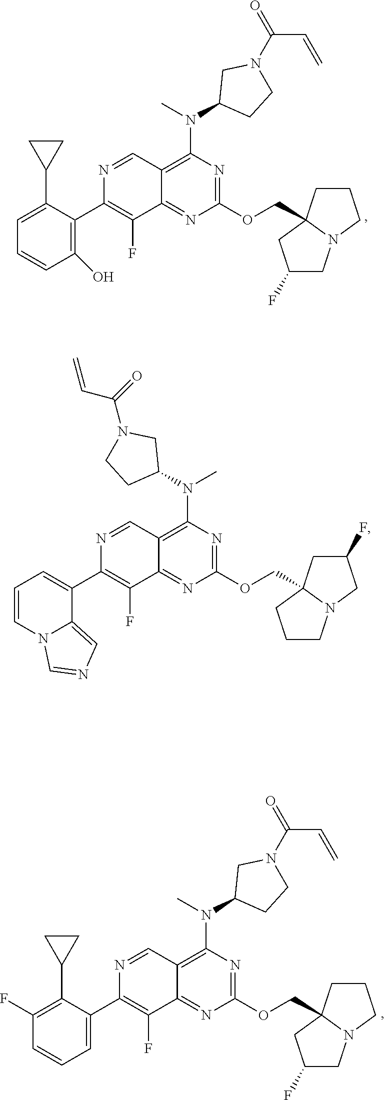 [SMILES: C=CC(=O)N1CC[C@@H](N(C)c2nc(OC[C@@]34CCCN3C[C@H](F)C4)nc3c(F)c(-c4c(O)cccc4C4CC4)ncc23)C1 ; C=CC(=O)N1CC[C@@H](N(C)c2nc(OC[C@@]34CCCN3C[C@H](F)C4)nc3c(F)c(-c4cccn5cncc45)ncc23)C1 ; C=CC(=O)N1CC[C@@H](N(C)c2nc(OC[C@@]34CCCN3C[C@H](F)C4)nc3c(F)c(-c4cccc(F)c4C4CC4)ncc23)C1]

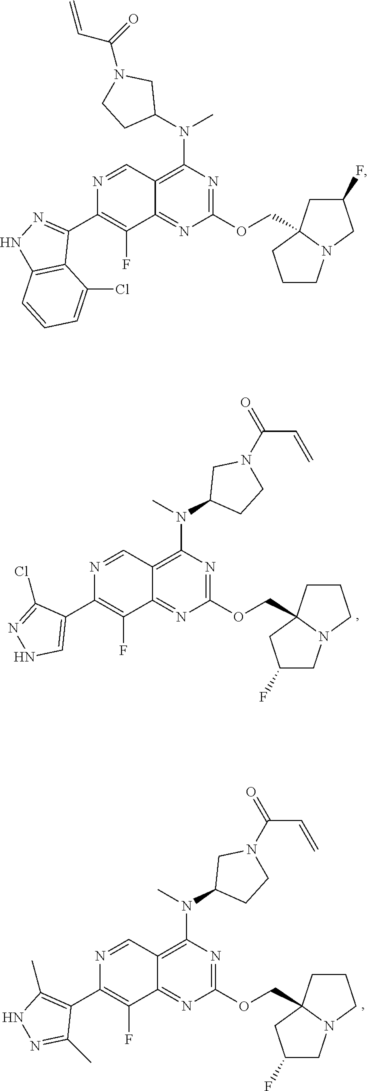 [SMILES: C=CC(=O)N1CCC(N(C)c2nc(OC[C@@]34CCCN3C[C@H](F)C4)nc3c(F)c(-c4n[nH]c5cccc(Cl)c45)ncc23)C1 ; C=CC(=O)N1CC[C@@H](N(C)c2nc(OC[C@@]34CCCN3C[C@H](F)C4)nc3c(F)c(-c4c[nH]nc4Cl)ncc23)C1 ; C=CC(=O)N1CC[C@@H](N(C)c2nc(OC[C@@]34CCCN3C[C@H](F)C4)nc3c(F)c(-c4c(C)n[nH]c4C)ncc23)C1]

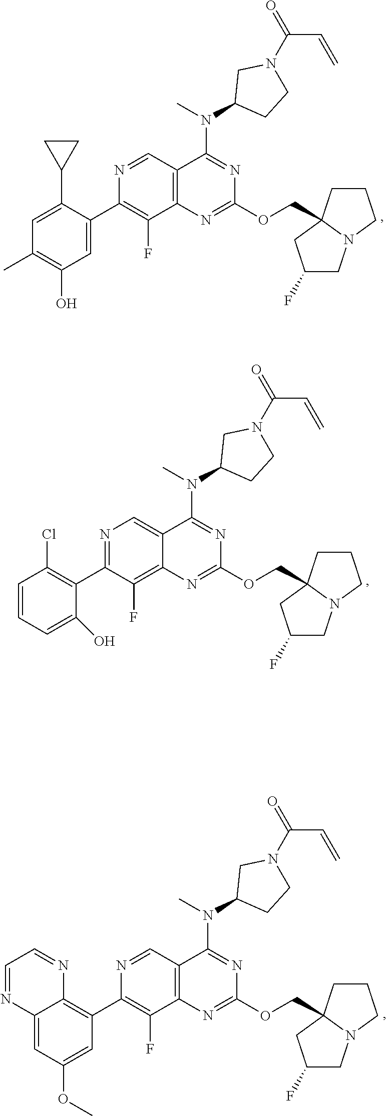 [SMILES: C=CC(=O)N1CC[C@@H](N(C)c2nc(OC[C@@]34CCCN3C[C@H](F)C4)nc3c(F)c(-c4cc(O)c(C)cc4C4CC4)ncc23)C1 ; C=CC(=O)N1CC[C@@H](N(C)c2nc(OC[C@@]34CCCN3C[C@H](F)C4)nc3c(F)c(-c4c(O)cccc4Cl)ncc23)C1 ; C=CC(=O)N1CC[C@@H](N(C)c2nc(OC[C@@]34CCCN3C[C@H](F)C4)nc3c(F)c(-c4cc(OC)cc5nccnc45)ncc23)C1]

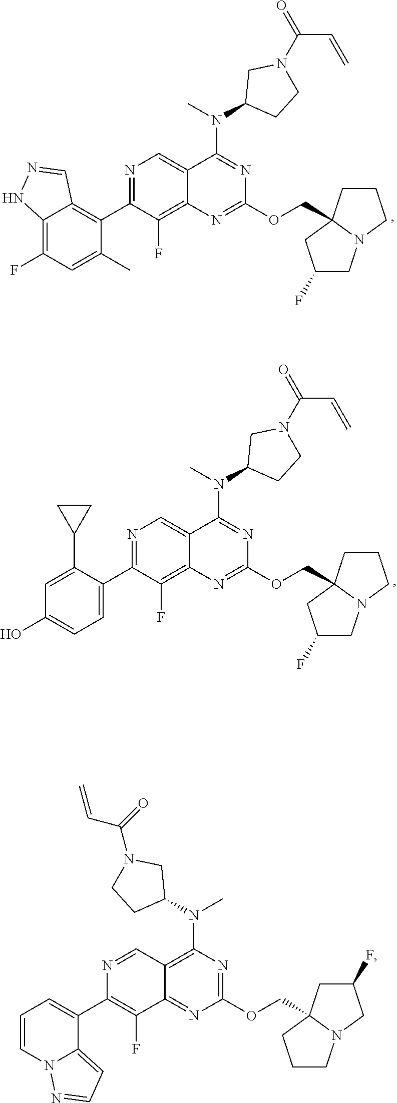 [SMILES: C=CC(=O)N1CC[C@@H](N(C)c2nc(OC[C@@]34CCCN3C[C@H](F)C4)nc3c(F)c(-c4c(C)cc(F)c5[nH]ncc45)ncc23)C1 ; C=CC(=O)N1CC[C@@H](N(C)c2nc(OC[C@@]34CCCN3C[C@H](F)C4)nc3c(F)c(-c4ccc(O)cc4C4CC4)ncc23)C1 ; C=CC(=O)N1CC[C@@H](N(C)c2nc(OC[C@@]34CCCN3C[C@H](F)C4)nc3c(F)c(-c4cccn5nccc45)ncc23)C1]

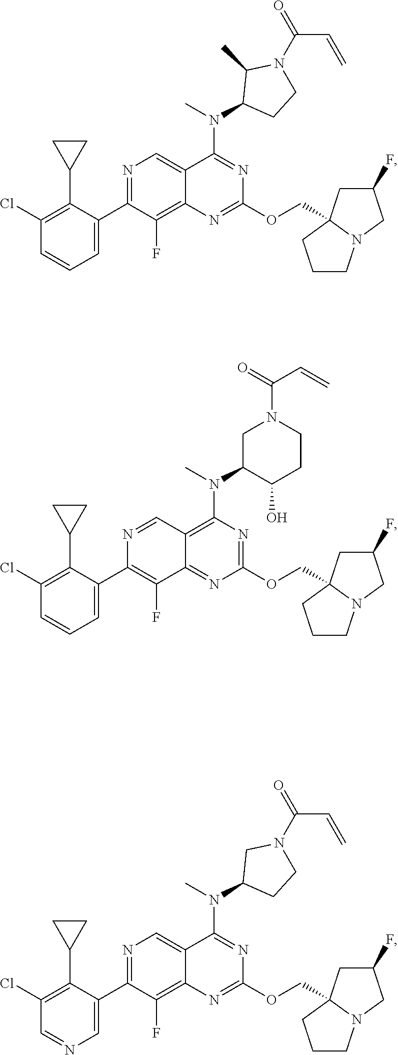 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C ; C=CC(=O)N1CC[C@H](O)[C@@H](N(C)c2nc(OC[C@@]34CCCN3C[C@H](F)C4)nc3c(F)c(-c4cccc(Cl)c4C4CC4)ncc23)C1 ; C=CC(=O)N1CC[C@@H](N(C)c2nc(OC[C@@]34CCCN3C[C@H](F)C4)nc3c(F)c(-c4cncc(Cl)c4C4CC4)ncc23)C1]

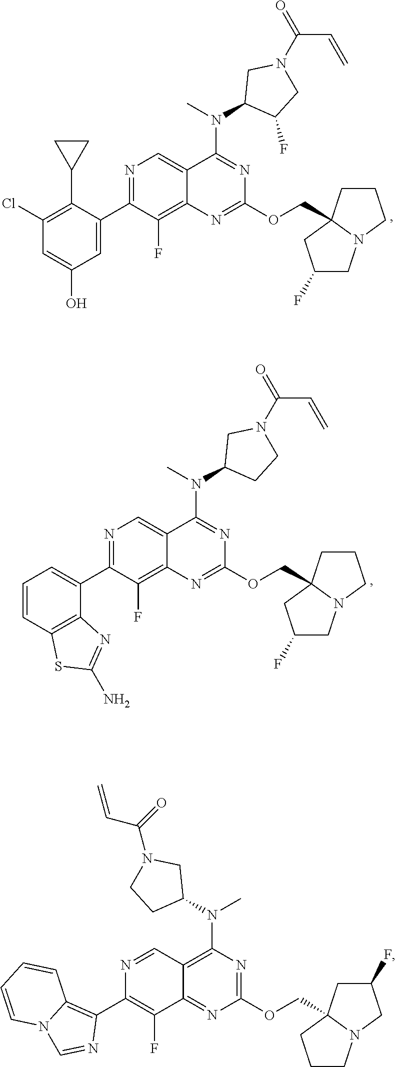 [SMILES: C=CC(=O)N1C[C@H](F)[C@@H](N(C)c2nc(OC[C@@]34CCCN3C[C@H](F)C4)nc3c(F)c(-c4cc(O)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cccc5sc(N)nc45)ncc23)C1 ; C=CC(=O)N1CC[C@@H](N(C)c2nc(OC[C@@]34CCCN3C[C@H](F)C4)nc3c(F)c(-c4ncn5ccccc45)ncc23)C1]

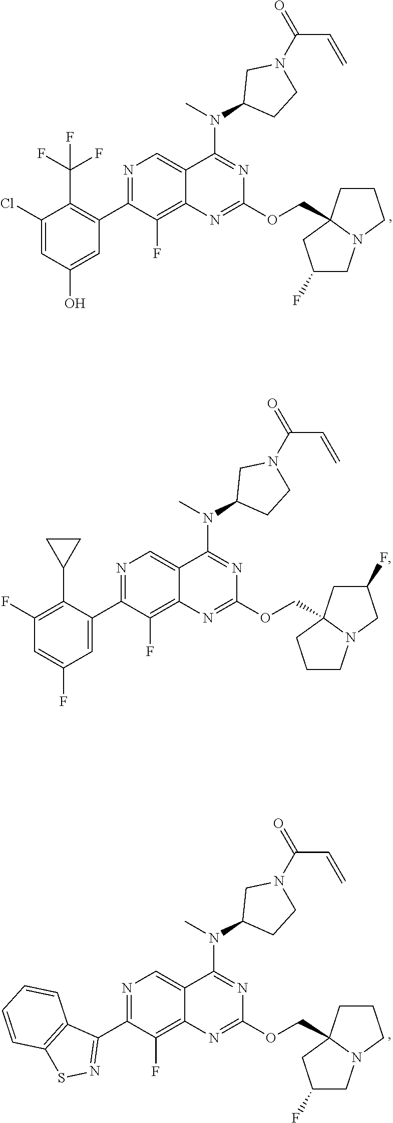 [SMILES: C=CC(=O)N1CC[C@@H](N(C)c2nc(OC[C@@]34CCCN3C[C@H](F)C4)nc3c(F)c(-c4cc(O)cc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(F)cc(F)c4C4CC4)ncc23)C1 ; C=CC(=O)N1CC[C@@H](N(C)c2nc(OC[C@@]34CCCN3C[C@H](F)C4)nc3c(F)c(-c4nsc5ccccc45)ncc23)C1]

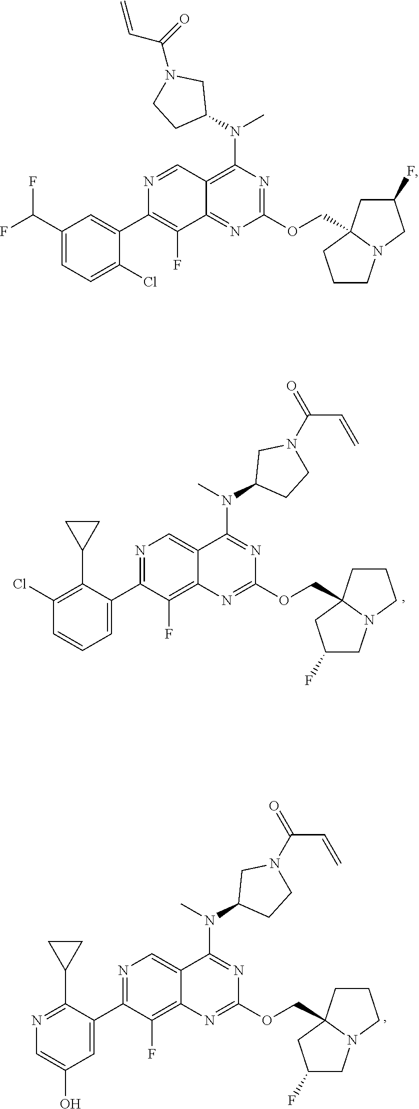 [SMILES: C=CC(=O)N1CC[C@@H](N(C)c2nc(OC[C@@]34CCCN3C[C@H](F)C4)nc3c(F)c(-c4cc(C(F)F)ccc4Cl)ncc23)C1 ; C=CC(=O)N1CC[C@@H](N(C)c2nc(OC[C@@]34CCCN3C[C@H](F)C4)nc3c(F)c(-c4cccc(Cl)c4C4CC4)ncc23)C1 ; C=CC(=O)N1CC[C@@H](N(C)c2nc(OC[C@@]34CCCN3C[C@H](F)C4)nc3c(F)c(-c4cc(O)cnc4C4CC4)ncc23)C1]

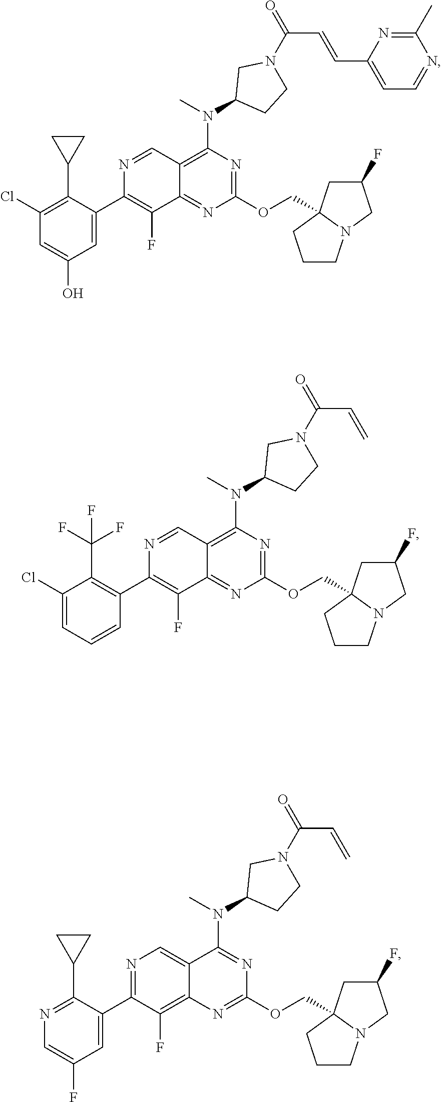 [SMILES: Cc1nccc(/C=C/C(=O)N2CC[C@@H](N(C)c3nc(OC[C@@]45CCCN4C[C@H](F)C5)nc4c(F)c(-c5cc(O)cc(Cl)c5C5CC5)ncc34)C2)n1 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)C1 ; C=CC(=O)N1CC[C@@H](N(C)c2nc(OC[C@@]34CCCN3C[C@H](F)C4)nc3c(F)c(-c4cc(F)cnc4C4CC4)ncc23)C1]

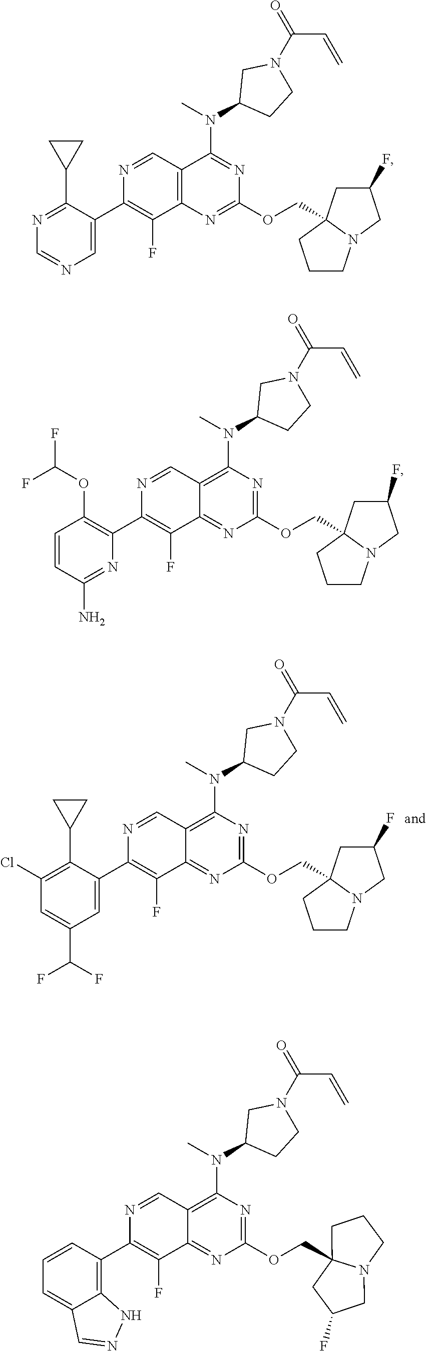 [SMILES: C=CC(=O)N1CC[C@@H](N(C)c2nc(OC[C@@]34CCCN3C[C@H](F)C4)nc3c(F)c(-c4cncnc4C4CC4)ncc23)C1 ; C=CC(=O)N1CC[C@@H](N(C)c2nc(OC[C@@]34CCCN3C[C@H](F)C4)nc3c(F)c(-c4nc(N)ccc4OC(F)F)ncc23)C1 ; C=CC(=O)N1CC[C@@H](N(C)c2nc(OC[C@@]34CCCN3C[C@H](F)C4)nc3c(F)c(-c4cc(C(F)F)cc(Cl)c4C4CC4)ncc23)C1 ; C=CC(=O)N1CC[C@@H](N(C)c2nc(OC[C@@]34CCCN3C[C@H](F)C4)nc3c(F)c(-c4cccc5cn[nH]c45)ncc23)C1]

and all salts and isotopologues thereof.

Embodiment 312. The compound of any one of embodiments 1-311, wherein the compound is not a salt.

Embodiment 313. The compound of any one of embodiments 1-311, wherein the compound is a salt.

Embodiment 314. The compound of embodiment 313, wherein the salt is a formate salt.

Embodiment 315. The compound of embodiments 313, wherein the salt is a trifluoroacetate salt.

Embodiment 316. The compound of embodiment 313 wherein the salt is a pharmaceutically acceptable salt.

Embodiment 317. A pharmaceutical formulation comprising the compound of any one of embodiments 1-313, wherein when the compound is a salt, the salt is a pharmaceutically acceptable salt, and a pharmaceutically acceptable carrier.

Embodiment 318. A method of treating or suppressing cancer comprising: administering a therapeutically effective amount of a compound of any one of embodiments 1-313, wherein when the compound is a salt, the salt is a pharmaceutically acceptable salt, or a pharmaceutical formulation according to embodiment 317, to a subject in need thereof.

Embodiment 319. The method of embodiment 318, wherein the cancer is selected from the group consisting of: lung, colorectal, pancreatic, bile duct, thyroid, gall bladder, uterine, mesothelioma, cervical, and bladder cancers.

Embodiment 320. The method of embodiment 318, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 321. The method of any one of embodiments 318-320, wherein the cancer is a KRAS G12C mediated cancer.

Embodiment 322. The method of any one of embodiments 318-320, wherein the subject has been diagnosed as having a KRAS G12C mediated cancer.

Embodiment 323. The method of any one of embodiments 318-322, wherein the method further comprises administering to the subject a therapeutically effective amount of an additional chemotherapeutic agent.

Embodiment 324. A compound of any one of embodiments 1-313 or the pharmaceutical formulation of embodiment 317 for use in a method of treating or suppressing cancer, wherein the method comprises administering a therapeutically effective amount of a compound of any one of embodiments 1-313, or of the pharmaceutical formulation of embodiment 317 wherein when the compound is a salt, the salt is a pharmaceutically acceptable salt, to a subject in need thereof.

Embodiment 325. The compound or pharmaceutical formulation for use of embodiment 323, wherein the cancer is selected from the group consisting of: lung, colorectal, pancreatic, bile duct, thyroid, gall bladder, uterine, mesothelioma, cervical, and bladder cancers.

Embodiment 326. The compound or pharmaceutical formulation for use of embodiment 325, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 327. The compound or pharmaceutical formulation for use of any one of embodiments 324-326, wherein the cancer is a KRAS G12C mediated cancer.

Embodiment 328. The compound or pharmaceutical formulation for use of any one of embodiments 324-327, wherein the subject has been diagnosed as having a KRAS G12C mediated cancer.

Embodiment 329. The compound or pharmaceutical formulation for use of any one of embodiments 324-328, wherein the method further comprises administering to the subject a therapeutically effective amount of an additional chemotherapeutic agent.

Embodiment 330. Use of a compound of any one of embodiments 1-313 or the pharmaceutical formulation of embodiment 318 in the manufacturing of a medicament for treating or suppressing cancer in a subject in need thereof.

Embodiment 331. The use of embodiment 330, wherein the cancer is selected from the group consisting of: lung, colorectal, pancreatic, bile duct, thyroid, gall bladder, uterine, mesothelioma, cervical, and bladder cancers.

Embodiment 332. The use of embodiment 331, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 333. The use of any one of embodiments 330-332, wherein the cancer is a KRAS G12C mediated cancer.

Embodiment 334. The use of any one of embodiments 330-333, wherein the subject has been diagnosed as having a KRAS G12C mediated cancer.

Embodiment 335. The use of any one of embodiments 330-334, wherein the compound or pharmaceutical formulation is configured for administration with an additional chemotherapeutic agent.

Embodiment 336. Use of a compound of any one of embodiments 1-313 or the pharmaceutical formulation of embodiment 318 for treating or suppressing cancer in a subject in need thereof.

Embodiment 337. The use of embodiment 336, wherein the cancer is selected from the group consisting of: lung, colorectal, pancreatic, bile duct, thyroid, gall bladder, uterine, mesothelioma, cervical, and bladder cancers.

Embodiment 338. The use of embodiment 336, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

Embodiment 339. The use of any one of embodiments 336-338, wherein the cancer is a KRAS G12C mediated cancer.

Embodiment 340. The use of any one of embodiments 336-339, wherein the subject has been diagnosed as having a KRAS G12C mediated cancer.

Embodiment 341. The use of any one of embodiments 336-340, wherein the compound or pharmaceutical formulation is configured for administration with an additional chemotherapeutic agent.

### General Synthetic Methods

Compounds of this disclosure can be made in view of the disclosure in the Examples shown below.

The starting materials and reagents used in preparing these compounds are either available from commercial suppliers such as MilliporeSigma, Bachem., etc. or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science-Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

### EXAMPLES

The following preparations of compounds of Formula (A), Formula (B) and Formula (C) and pharmaceutically acceptable salts thereof are given to enable those skilled in the art to more clearly understand and to practice the present disclosure. They should not be considered as limiting the scope of the disclosure, but merely as being illustrative and representative thereof.

Compounds 1-264 in Table 1 were prepared using methods described in the synthetic examples or variations thereof that are accessible to those of skill in the art.

The following abbreviations are used in this section:

| | |
|---|---|
| ACN | Acetonitrile |
| Boc | tert-butyloxycarbonyl |
| DCM | dichloromethane |
| ESI+ | Electrospray ionization |
| FA | Formic acid |
| h | Hour(s) |
| HPLC | High performance liquid chromatography |
| LCMS | Liquid chromatography-mass spectrometry |
| m/z | Mass to charge ratio |
| min | Minute(s) |
| OTf | trifluoromethanesulfonate |
| Rt | Retention time |
| SFC | Supercritical fluid chromatography |
| TFA | Trifluoroacetic acid |

All reagents were obtained from commercial suppliers and used without further purification unless otherwise stated.

### SYNTHETIC EXAMPLES

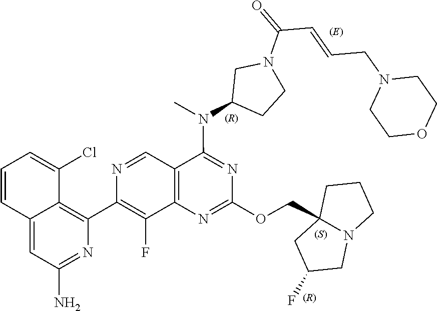

### Example 1: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-morpholinobut-2-en-1-one

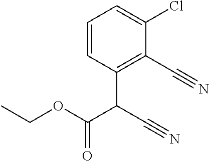 [SMILES: CCOC(=O)C(C#N)c1cccc(Cl)c1C#N]

### Step 1: ethyl 2-(3-chloro-2-cyano-phenyl)-2-cyano-acetate

To a solution of sodium hydride (5.14 g, 128.57 mmol, 60% purity) in N,N-dimethylformaldehyde (30 mL) was added ethyl 2-cyanoacetate (14.54 g, 128.57 mmol) at 0° C. The mixture was stirred at 25° C. for 1 h, followed by the addition of 2-chloro-6-fluoro-benzonitrile (10 g, 4.29 mmol) and the mixture was stirred at 50° C. for 4 h. The reaction mixture was quenched with saturated ammonium chloride (20 mL) and extracted with ethyl acetate (2×20 mL). The combined organic layers were dried over sodium sulfate and concentrated in vacuo affording ethyl 2-(3-chloro-2-cyano-phenyl)-2-cyano-acetate (16 g, crude) as a yellow solid, used in next step without further purification. LCMS Rt=0.812 min, m/z=248.0 [M+H]⁺.

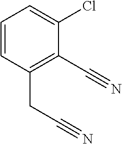 [SMILES: N#CCc1cccc(Cl)c1C#N]

### Step 2: 2-chloro-6-(cyanomethyl)benzonitrile

A solution of ethyl 2-(3-chloro-2-cyano-phenyl)-2-cyano-acetate (15 g, 60.32 mmol) in dimethyl sulfoxide (20 mL) and water (2 mL) was stirred at 100° C. for 6 h. The reaction mixture was diluted with water (10 mL) and the resulting precipitate was filtered and dried affording 2-chloro-6-(cyanomethyl)benzonitrile (9.4 g, crude) as a white solid, used in the next step without further purification: ¹H NMR (400 MHz, Dimethylsulfoxide-d6) δ 7.80-7.68 (m, 2H), 7.65-7.59 (m, 1H), 4.33 (s, 2H). LCMS Rt=0.680 min, m/z=176.0 [M+H]⁺.

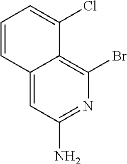 [SMILES: Nc1cc2cccc(Cl)c2c(Br)n1]

### Step 3: 1-bromo-8-chloro-isoquinolin-3-amine

A solution of 2-chloro-6-(cyanomethyl)benzonitrile (9.4 g, 53.23 mmol) in hydrobromic acid in acetic acid (316.06 g, 1.29 mol, 33% purity) was stirred at 25° C. for 12 h. The reaction mixture was quenched with saturated sodium bicarbonate (300 mL). The resulting precipitate was filtered and dried affording 1-bromo-8-chloro-isoquinolin-3-amine (13 g, crude) as a yellow solid used in the next step without further purification: ¹H NMR (400 MHz, dimethyl sulfoxide-d6) δ 8.47-8.46 (m, 1H), 8.01-7.96 (m, 1H), 7.74-7.71 (m, 1H), 7.68-7.63 (m, 1H).

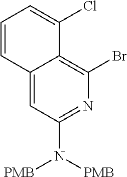

### Step 4: 1-bromo-8-chloro-N,N-bis[(4-methoxyphenyl)methyl]isoquinolin-3-amine

The PMB protection reaction was prepared in a similar fashion to Example #71, Step 1. The crude product was purified by column chromatography (silica gel, 100-200 mesh, 0-10% ethyl acetate in petroleum ether) affording 1-bromo-8-chloro-N,N-bis[(4-methoxyphenyl)methyl]isoquinolin-3-amine (3 g, 31.04%) as a yellow solid: ¹H NMR (400 MHz, Chloroform-d) δ 7.33-7.31 (m, 2H), 7.24-7.18 (m, 5H), 6.86 (d, J=8.6 Hz, 4H), 6.49-6.45 (m, 1H), 4.74 (s, 4H), 3.80 (s, 6H). LCMS Rt=3.172 min, m/z=498.1 [M+H]⁺.

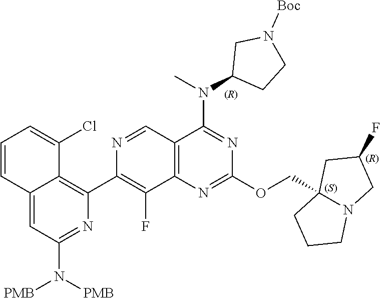

### Step 5: (R)-tert-butyl 3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by column chromatography (silica gel, 100-200 mesh, 0-100% ethyl acetate in petroleum ether) affording (R)-tert-butyl 3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (590 mg, 50.81%) as a yellow solid. LCMS Rt=0.803 min, m/z=920.4 [M+H]⁺.

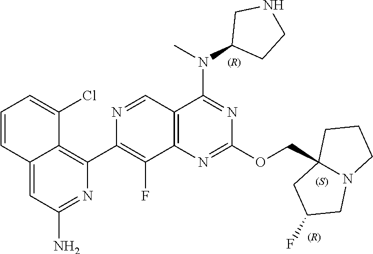

### Step 6: 7-(3-amino-8-chloroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The de-Boc and PMB protecting reaction was prepared in a similar fashion to Example #71, Step 7. The residue was purified by reverse phase HPLC (column: Phenomenex Luna 80*30 mm*3 um; mobile phase: [water (trifluroacetic acid)-acetonitrile]; B %: 5%-35%, 8 min) affording 7-(3-amino-8-chloroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (65 mg, 59.08%, trifluoroacetic salt) as a yellow solid. LCMS Rt=0.456 min, m/z=580.2 [M+H]⁺.

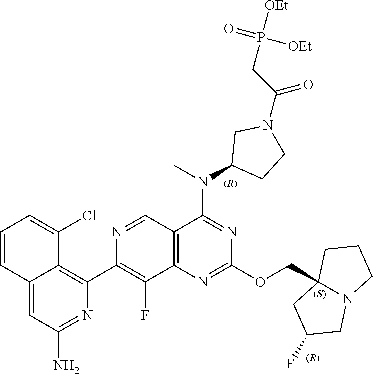

### Step 7: diethyl (2-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate

The amide coupling reaction was prepared in a similar fashion to Example #2, Step 5. The crude product was purified by reverse phase HPLC (column: Waters Xbridge BEH C18 100*30 mm*10 um; mobile phase: [water (NH4HCO3)-acetonitrile]; B %: 20%-60%, 8 min) affording diethyl (2-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate (15 mg, 22.96%) as a yellow solid. LCMS Rt=0.713 min, m/z=758.3 [M+H]⁺.

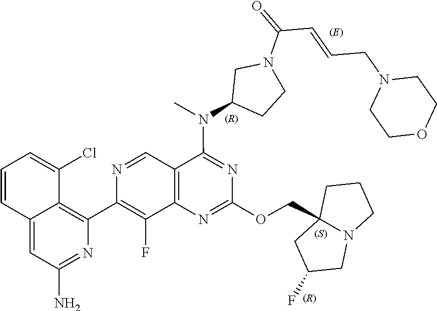

### Step 8: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-morpholinobut-2-en-1-one

The Horner-Wadsworth-Emmons reaction was prepared in a similar fashion to Example #2, Step 6. The crude product was purified by reverse phase HPLC(column: Waters Xbridge BEH C18 100*30 mm*10 um; mobile phase: [water (NH4HCO3)-acetonitrile]; B %: 10%-50%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-morpholinobut-2-en-1-one (8.76 mg, 87.80%) as a yellow amorphous solid: ¹H NMR (400 MHz, Acetonitrile-d3) δ 9.17-9.16 (m, 1H), 7.63 (d, J=8.4 Hz, 1H), 7.41 (t, J=7.9 Hz, 1H), 7.28-7.18 (m, 1H), 6.95-6.89 (m, 1H), 6.80-6.64 (m, 1H), 6.48-6.33 (m, 1H), 5.44-5.17 (m, 2H), 5.17-5.10 (m, 2H), 4.24-4.17 (m, 1H), 4.16-4.08 (m, 1H), 4.08-3.89 (m, 1H), 3.88-3.76 (m, 1H), 3.68-3.58 (m, 5H), 3.57-3.43 (m, 1H), 3.40 (br s, 3H), 3.20-3.01 (m, 5H), 2.94-2.84 (m, 1H), 2.40 (br s, 4H), 2.32-2.17 (m, 2H), 2.12-2.02 (m, 3H), 1.92-1.75 (m, 3H). LCMS Rt=2.418 min, m/z=733.3 [M+H]⁺. LCMS (5 to 95% acetonitrile in water+0.03% ammonium bicarbonate over 6 mins) retention time 2.418 min, ESI+ found [M+H]=733.3.

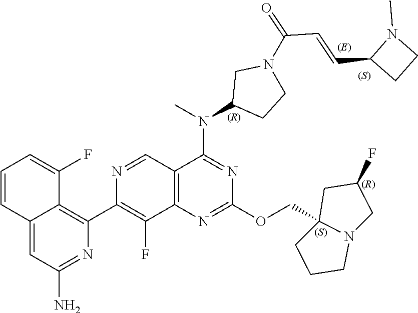

### Example 2: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one

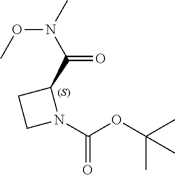

### Step 1: (S)-tert-butyl 2-(methoxy(methyl)carbamoyl)azetidine-1-carboxylate

To a solution of (2S)-1-tert-butoxycarbonylazetidine-2-carboxylic acid (2 g, 9.94 mmol) and N-methoxymethanamine;hydrochloride (1.16 g, 11.93 mmol) in N,N-dimethylformaldehyde (20 mL) were added 4-methylmorpholine (1.21 g, 11.93 mmol), 1-hydroxybenzotriazole (1.61 g, 11.93 mmol, 1.2 eq) and 3-(ethyliminomethylideneamino)-N,N-dimethylpropan-1-amine (2.29 g, 11.93 mmol) at 0° C., the mixture was stirred at 20° C. for 12 h. The mixture was diluted with water (20 mL) and extracted with ethyl acetate (3×50 mL). The combined organic layers were dried over sodium sulphate and concentrated in vacuo. The residue was purified by column chromatography (silica gel, 100-200 mesh, 0-100% ethyl acetate in petroleum ether) affording (S)-tert-butyl 2-(methoxy(methyl)carbamoyl)azetidine-1-carboxylate (2.1 g, 85.62%) as a colorless solid: ¹H NMR (400 MHz, Chloroform-d) δ 5.10-4.95 (m, 1H), 4.09-3.99 (m, 1H), 3.92-3.81 (m, 1H), 3.70 (s, 3H), 3.30-3.17 (m, 3H), 2.46 (dtd, J=6.4, 9.0, 11.1 Hz, 1H), 2.19-2.06 (m, 1H), 1.42 (s, 9H).

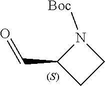

### Step 2: (S)-tert-butyl 2-formylazetidine-1-carboxylate

To a solution of tert-butyl (2S)-2-[methoxy(methyl)carbamoyl]azetidine-1-carboxylate (170 mg, 695.90 umol) in tetrahydrofuran (2 mL) was added bis(2-methylpropyl)alumanylium; hydride (1 M, 1.39 mL) (in Toluene) at 0° C. under nitrogen atmosphere. The mixture was stirred at 25° C. for 0.5 h under nitrogen atmosphere. The mixture was quenched with 5% potassium hydrogen sulfate (2 mL) at 0° C., and extracted with ethyl acetate (10 mL). The combined organic layers were washed with saturated sodium bicarbonate (2 mL) and brine (3 mL), dried over sodium sulphate and concentrated in vacuo affording (S)-tert-butyl 2-formylazetidine-1-carboxylate (90 mg, crude) as a white solid used in next step without any further purification.

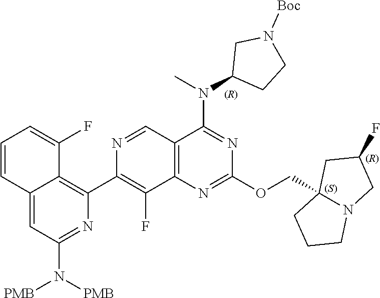

### Step 3: (R)-tert-butyl 3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by column chromatography (silica gel, 100-200 mesh, 0-100% ethyl acetate in petroleum ether) affording (R)-tert-butyl 3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (1.5 g, 73.08%) as a brown oil. LCMS Rt=2.675 min, m/z=904.4 [M+H]⁺.

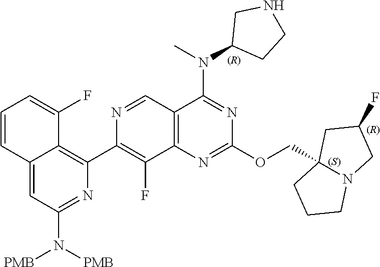

### Step 4: 7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

A mixture of (R)-tert-butyl 3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (270 mg, 298.34 umol) in hydrochloric acid in ethyl acetate (2 mL, 4M) was stirred at 20° C. for 0.5 h. The reaction mixture was concentrated to dryness in vacuo affording 7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (270 mg, crude, hydrochloride salt) as a yellow solid, used in next step without any further purification. LCMS Rt=0.896 min, m/z=804.4 [M+H]⁺.

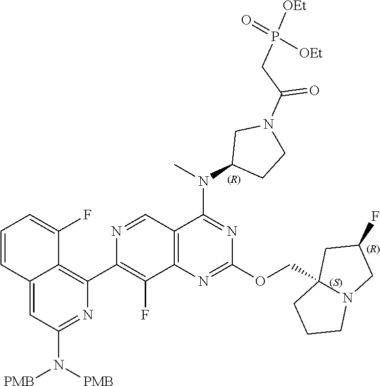

### Step 5: diethyl (2-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate

To a solution of 7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (270 mg, 320.91 umol, hydrochloric acid salt), 2-diethoxyphosphorylacetic acid (125.88 mg, 641.82 umol) and N,N-diisopropylethylamine (124.43 mg, 962.73 umol) in dichloromethane (3 mL) was added 2,4,6-tripropyl-1,3,5,2,4,6-trioxatriphosphinane 2,4,6-trioxide (408.43 mg, 641.82 umol, 50% purity in ethyl acetate). The mixture was stirred at 0° C. for 1 h. The reaction mixture was quenched by 1N HCl (5 mL) at 0° C., and extracted with dichloromethane (3×5 mL). The combined organic layers were dried over sodium sulphate and concentrated in vacuo. The residue was purified by column chromatography (silica gel, 100-200 mesh, 0-20% methanol in dichloromethane) affording diethyl (2-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate (300 mg, 95.10%) as a brown oil. LCMS Rt=0.753 min, m/z=982.4 [M+H]+.

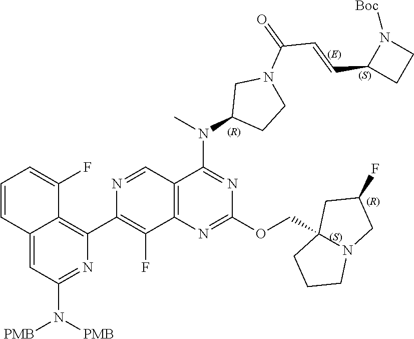

### Step 6: (S)-tert-butyl 2-((E)-3-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

To a solution of diethyl (2-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate (150 mg, 152.59 umol), N-ethyl-N-isopropylpropan-2-amine (59.16 mg, 457.77 umol), lithium chloride (32.34 mg, 762.95 umol) in acetonitrile (2 mL) was added (S)-tert-butyl 2-formylazetidine-1-carboxylate (84.79 mg, 457.77 umol). The mixture was stirred at 25° C. for 12 h. The reaction mixture was filtered and the filtrate was concentrated to dryness in vacuo. The residue was purified by column chromatography (silica gel, 100-200 mesh, 0-20% methanol in dichloromethane) affording (S)-tert-butyl 2-((E)-3-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (90 mg, 58.16%) as a white solid. LCMS Rt=1.173 min, m/z=1013.5 [M+H]⁺.

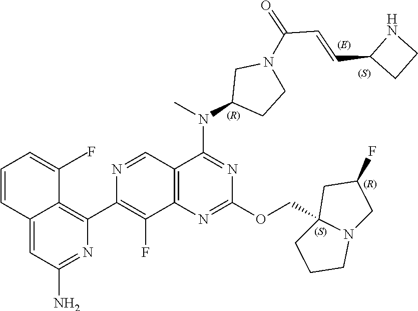

### Step 7: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one

To a solution of (S)-tert-butyl 2-((E)-3-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (80 mg, 78.88 umol) in dichloromethane (2 mL) was added trifluoroacetic acid (1 mL). The mixture was stirred at 25° C. for 1 h. The reaction mixture was filtered and the filtrate was concentrated to dryness in vacuo. The residue was purified by reverse phase HPLC: column: Phenomenex Luna 80*30 mm*3 um; mobile phase: [water (trifluroacetic acid)-acetonitrile]; B %: 5%-35%, 8 min affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one (20 mg, 32.18%, trifluoroacetic salt) as a yellow solid. LCMS Rt=0.626 min, m/z=673.3 [M+H]⁺.

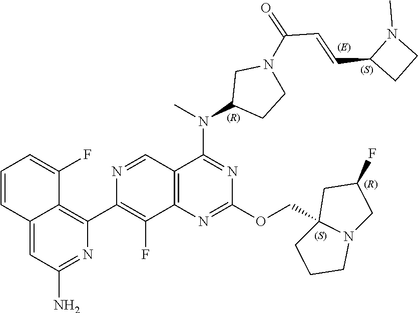

### Step 8: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one

To a solution of (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one (12.7 mg, 16.12 umol, trifluoroacetic salt) in methanol (1 mL) was added triethylamine (4.89 mg, 48.37 umol), acetic acid (968.15 ug, 16.12 umol), formaldehyde (915.82 ug, 11.29 umol) and sodium cyanoboranuide (2.53 mg, 40.30 umol). The mixture was stirred at 25° C. for 1 h. The reaction mixture was filtered and the filtrate was concentrated to dryness in vacuo. The residue was purified by reverse phase HPLC (column: Waters Xbridge BEH C18 100*30 mm*10 um; mobile phase: [water (NH4HCO3)-acetonitrile]; B %: 18%-58%, 8 min) affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one (1 mg, 8.50%) as a white solid: ¹H NMR (400 MHz, Acetonitrile-d3) δ 9.28 (d, J=2.8 Hz, 1H), 7.60-7.50 (m, 2H), 6.98 (d, J=2.4 Hz, 1H), 6.94-6.80 (m, 2H), 6.61-6.44 (m, 1H), 5.51-5.22 (m, 4H), 4.37-4.25 (m, 2H), 4.24-4.06 (m, 1H), 4.05-3.85 (m, 2H), 3.85-3.71 (m, 2H), 3.70-3.53 (m, 2H), 3.52-3.34 (m, 5H), 3.29 (br d, J=11.1 Hz, 2H), 3.25-3.20 (m, 1H), 3.07-2.97 (m, 2H), 2.28-2.05 (m, 7H), 1.99-1.82 (m, 3H). LCMS Rt=1.774 min, m/z=687.3 [M+H]⁺.

LCMS (5 to 95% acetonitrile in water+0.03% formic acid over 6 min) retention time 1.774 min, ESI+ found [M+H]=687.3.

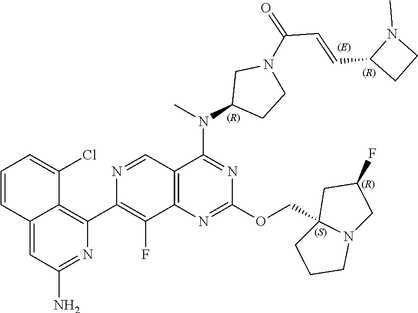

### Example 3: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one

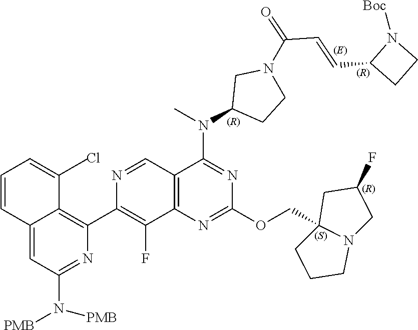

### Step 1: (R)-tert-butyl 2-((E)-3-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

The Horner-Wadsworth-Emmons reaction was prepared in a similar fashion to Example #2, Step 6. The reaction mixture was diluted with water (10 mL) and extracted with dichloromethane (2×20 mL). The combined organic layers were dried over sodium sulphate and concentrated in vacuo affording (R)-tert-butyl 2-((E)-3-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (155 mg, crude) as an orange gum used in next step without further purification. LCMS Rt=0.459 min, m/z=1029.5 [M+H]⁺.

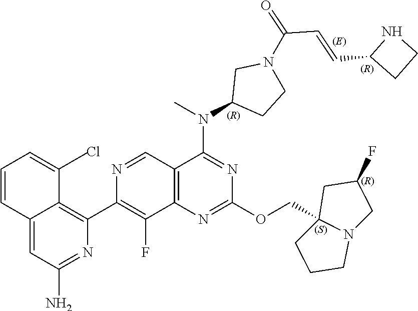

### Step 2: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-azetidin-2-yl)prop-2-en-1-one

The de-Boc and PMB protecting reaction was prepared in a similar fashion to Example #2, Step 7. The residue was purified by reverse phase HPLC (column: Phenomenex Luna C18 150*30 mm*5 um; mobile phase: [water(trifluoroacetic acid)-acetonitrile]; B %: 10%-40%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-azetidin-2-yl)prop-2-en-1-one (40 mg, 29.86%, trifluoroacetic salt) as a black solid. LCMS Rt=0.601 min, m/z=689.3 [M+H]⁺.

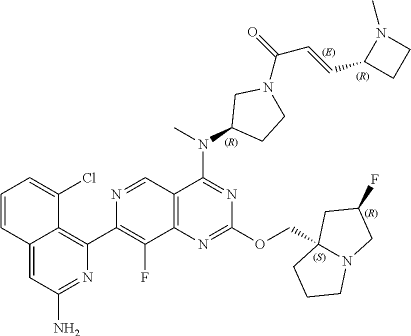

### Step 3: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one

The reductive amination reaction was prepared in a similar fashion to Example #2, Step 8. The residue was purified by reverse phase HPLC (column: Waters Xbridge BEH C18 100*30 mm*10 um; mobile phase: [water (NH4HCO3)-acetonitrile]; B %: 25%-55%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one (2.7 mg, 8.32%) as a yellow amorphous solid: ¹H NMR (400 MHz, Acetonitrile-d3) δ 9.21-9.10 (m, 1H), 7.71-7.59 (m, 1H), 7.41 (t, J=7.9 Hz, 1H), 7.25-7.14 (m, 1H), 6.96-6.83 (m, 1H), 6.80-6.69 (m, 1H), 6.45-6.30 (m, 1H), 5.45-5.30 (m, 2H), 5.17-5.29 (m, 2H), 4.57-4.26 (m, 1H), 4.16-4.08 (m, 2H), 3.97-3.71 (m, 2H), 3.69-3.47 (m, 3H), 3.41-3.37 (m, 3H), 3.29-3.22 (m, 1H), 3.13 (br d, J=8.3 Hz, 2H), 3.08-3.04 (m, 1H), 2.90-2.85 (m, 1H), 2.28-2.20 (m, 3H), 2.19-2.01 (m, 5H), 1.92-1.75 (m, 5H). LCMS Rt=2.704 min, m/z=703.3 [M+H]⁺. LCMS (5 to 95% acetonitrile in water+0.03% ammonium bicarbonate over 6 mins) retention time 2.704 min, ESI+ found [M+H]=703.3.

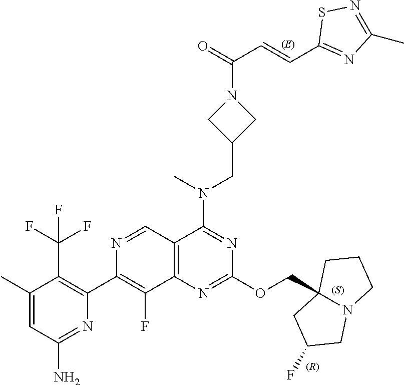

### Example 4: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-methyl-1,2,4-thiadiazol-5-yl)prop-2-en-1-one

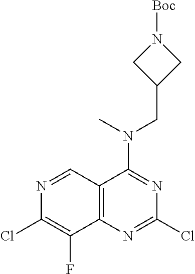 [SMILES: CN(CC1CN(C(=O)OC(C)(C)C)C1)c1nc(Cl)nc2c(F)c(Cl)ncc12]

### Step 1: tert-butyl 3-[[(2,7-dichloro-8-fluoro-pyrido[4,3-d]pyrimidin-4-yl)-methyl-amino]methyl]azetidine-1-carboxylate

The substitution reaction was prepared in a similar fashion to Example #71, Step 3. The mixture was concentrated in vacuo affording tert-butyl 3-[[(2,7-dichloro-8-fluoro-pyrido[4,3-d]pyrimidin-4-yl)-methyl-amino]methyl]azetidine-1-carboxylate (7 g, crude) as a brown solid, used in the next step without further purification. LCMS Rt=0.826 min, m/z=415.1 [M+H]⁺.

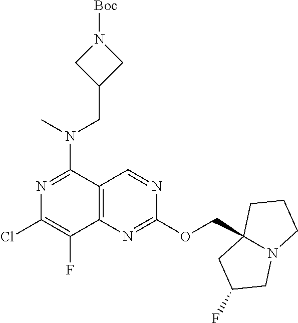 [SMILES: CN(CC1CN(C(=O)OC(C)(C)C)C1)c1nc(Cl)c(F)c2nc(OC[C@@]34CCCN3C[C@H](F)C4)ncc12]

### Step 2: tert-butyl 3-(((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The substitution reaction was prepared in a similar fashion to Example #71, Step 4. The crude product was purified by column chromatography (silica gel, 100-200 mesh, 80-100% ethyl acetate in petroleum ether) affording tert-butyl 3-(((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (2 g, 44.12%) as a yellow solid: ¹H NMR (400 MHz, Chloroform-d) δ 8.87 (s, 1H), 5.41-5.18 (m, 1H), 4.30-4.19 (m, 2H), 4.08 (q, J=8.3 Hz, 4H), 3.81-3.74 (m, 2H), 3.53-3.48 (m, 3H), 3.35-3.17 (m, 3H), 3.09-2.95 (m, 2H), 2.29-2.08 (m, 3H), 2.00-1.84 (m, 3H), 1.45 (s, 9H). LCMS Rt=0.645 min, m/z=538.2 [M+H]⁺.

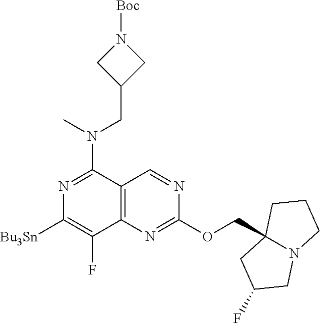

### Step 3: tert-butyl 3-(((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)methyl)azetidine-1-carboxylate

The tin reagent formation was prepared in a similar fashion to Example #71, Step 5. The crude product was purified by column chromatography (silica gel, 100-200 mesh, 80-100% ethyl acetate in petroleum ether) affording tert-butyl 3-(((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)methyl)azetidine-1-carboxylate (2.5 g, 84.90%) as a yellow oil. LCMS Rt=2.437 min, m/z=794.4 [M+H]⁺.

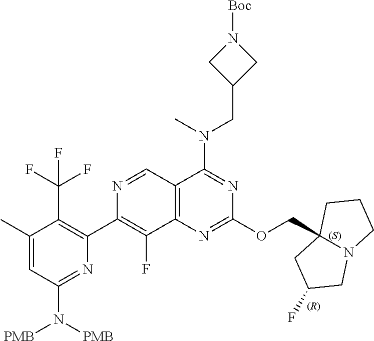

### Step 4: tert-butyl 3-(((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The crude product was purified by column chromatography (silica gel, 100-200 mesh, 80-100% ethyl acetate in petroleum ether) affording tert-butyl 3-(((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (1.3 g, 44.91%) as a yellow solid. LCMS Rt=0.810 min, m/z=918.4 [M+H]⁺.

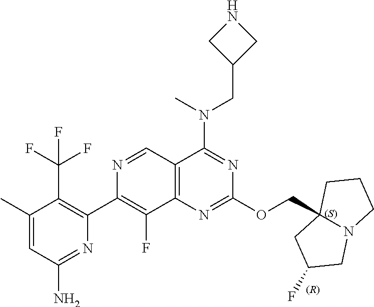

### Step 5: 7-(6-amino-4-methyl-3-(trifluoromethyl)pyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The mixture was purified by reverse phase HPLC(column: Phenomenex Luna 80*30 mm*3 um; mobile phase: [water (trifluroacetic acid)-acetonitrile]; B %: 35%-75%, 8 min) affording 7-(6-amino-4-methyl-3-(trifluoromethyl)pyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (700 mg, 71.45%, trifluoroacetate salt) as a yellow solid. LCMS Rt=0.594 min, m/z=578.3 [M+H]⁺.

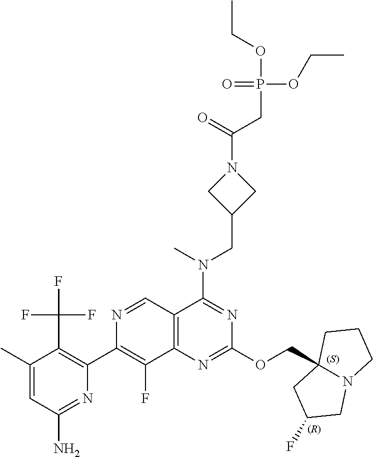

### Step 6: diethyl (2-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-2-oxoethyl)phosphonate

The amide coupling reaction was prepared in a similar fashion to Example #2, Step 5. The crude product was purified by reverse phase HPLC (column: Waters Xbridge BEH C18 100*30 mm*10 um; mobile phase: [water (NH4HCO3)-acetonitrile]; B %: 40%-70%, 8 min) affording diethyl (2-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-2-oxoethyl)phosphonate (120 mg, 36.61%) as a colorless oil. LCMS Rt=0.616 min, m/z=756.3 [M+H]⁺.

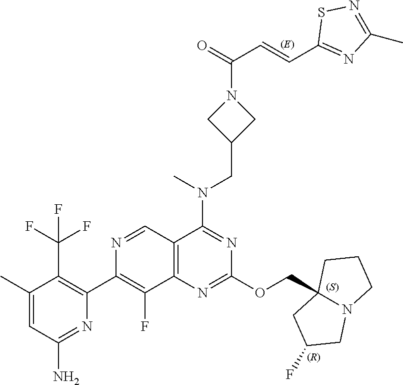

### Step 7: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-methyl-1,2,4-thiadiazol-5-yl)prop-2-en-1-one

The Horner-Wadsworth-Emmons reaction was prepared in a similar fashion to Example #2, Step 6. The crude product was purified by reverse phase HPLC (column: Waters Xbridge BEH C18 100*30 mm*10 um; mobile phase: [water (NH4HCO3)-acetonitrile]; B %: 40%-70%, 8 min) affording (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-methyl-1,2,4-thiadiazol-5-yl)prop-2-en-1-one (19.39 mg, 23.10%) as a yellow solid: ¹H NMR (400 MHz, Acetonitrile-d3) δ 9.22-9.16 (m, 1H), 7.72-7.62 (m, 1H), 7.11-6.99 (m, 1H), 6.60 (br s, 1H), 5.34-5.16 (m, 1H), 4.48 (br t, J=8.3 Hz, 1H), 4.30-4.24 (m, 2H), 4.21 (br dd, J=3.8, 10.3 Hz, 2H), 4.11 (br s, 2H), 3.96 (br dd, J=5.3, 10.4 Hz, 1H), 3.58 (br s, 3H), 3.23 (br d, J=7.6 Hz, 2H), 3.19-3.14 (m, 2H), 2.91-2.85 (m, 1H), 2.66-2.59 (m, 3H), 2.43 (br s, 3H), 2.21-2.03 (m, 3H), 1.91-1.78 (m, 3H). LCMS Rt=2.760 min, m/z=730.3 [M+H]⁺.

LCMS (5 to 95% acetonitrile in water+0.03% ammonium bicarbonate over 6 mins) retention time 2.760 min, ESI+ found [M+H]=730.3.

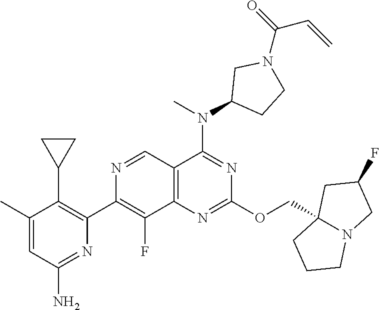 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1]

### Example 5: 1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

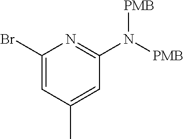

### Step 1: 6-bromo-N,N-bis[(4-methoxyphenyl)methyl]-4-methyl-pyridin-2-amine

The PMB protection reaction was prepared in a similar fashion to Example #71, Step 1. The crude product was washed by tert-butyl methyl ether (30 mL), then filtered affording 6-bromo-N,N-bis[(4-methoxyphenyl)methyl]-4-methyl-pyridin-2-amine (21 g, 58.21%) as a white solid. ¹H NMR (400 MHz, Chloroform-d) δ 7.20-7.14 (m, 4H), 6.90-6.82 (m, 4H), 6.62-6.58 (m, 1H), 6.20-6.15 (m, 1H), 4.69-4.62 (m, 4H), 3.86-3.76 (m, 6H), 2.17-2.10 (m, 3H). LCMS Rt=0.966 min, m/z=426.1 [M+H]⁺.

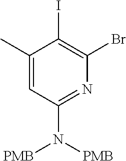

### Step 2: 6-bromo-5-iodo-N,N-bis[(4-methoxyphenyl)methyl]-4-methyl-pyridin-2-amine

To a solution of 6-bromo-N,N-bis[(4-methoxyphenyl)methyl]-4-methyl-pyridin-2-amine (11 g, 25.74 mmol) in N,N-dimethylformaldehyde (50 mL) was added N-iodo-succinimide (8.69 g, 38.61 mmol), the reaction was stirred at 20° C. for 1 h. The reaction mixture was diluted with water (30 mL) and extracted with dichloromethane (2×30 mL). The combined organic layers were dried over sodium sulphate and concentrated under vacuo. The crude product was washed by ethyl acetate (15 mL), affording 6-bromo-5-iodo-N,N-bis[(4-methoxyphenyl)methyl]-4-methyl-pyridin-2-amine (10 g, 66.71%) as a yellow solid. ¹H NMR (400 MHz, Chloroform-d) δ 7.20-7.13 (m, 4H), 6.91-6.83 (m, 4H), 6.31-6.26 (m, 1H), 4.70-4.59 (m, 4H), 3.87-3.78 (m, 6H), 2.36-2.31 (m, 3H). LCMS Rt=1.025 min, m/z=552.0 [M+H]⁺.

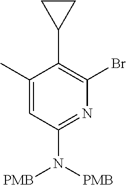

### Step 3: 6-bromo-5-cyclopropyl-N,N-bis[(4-methoxyphenyl)methyl]-4-methyl-pyridin-2-amine

To a solution of 6-bromo-5-iodo-N,N-bis[(4-methoxyphenyl)methyl]-4-methyl-pyridin-2-amine (8 g, 14.46 mmol) in tert-Amyl Alcohol (80 mL) was added cyclopropylboronic acid (1.28 g, 14.89 mmol), cesium carbonate (14.13 g, 43.38 mmol) and (1,1′-bis(diphenylphosphino)ferrocene)palladium(II) dichloride (1.06 g, 1.45 mmol), the reaction was stirred at 90° C. under nitrogen atmosphere for 12 h. The reaction mixture was diluted with water (50 mL) and extracted with dichloromethane (2×50 mL). The combined organic layers were dried over sodium sulphate and concentrated under vacuo affording 6-bromo-5-cyclopropyl-N,N-bis[(4-methoxyphenyl)methyl]-4-methyl-pyridin-2-amine (2.2 g, 26.04%) as a white solid. LCMS Rt=1.011 min, m/z=466.1 [M+H]⁺.

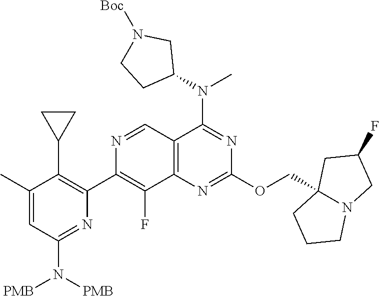

### Step 4: (R)-tert-butyl 3-((7-(6-(bis(4-methoxybenzyl)amino)-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by column chromatography (silica gel, 100-200 mesh, 0%-40% tetrahydrofuran in petroleum ether) affording (R)-tert-butyl 3-((7-(6-(bis(4-methoxybenzyl)amino)-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (1.3 g, 90.97%) as a yellow oil. LCMS Rt=2.371 min, m/z=890.5 [M+H]⁺.

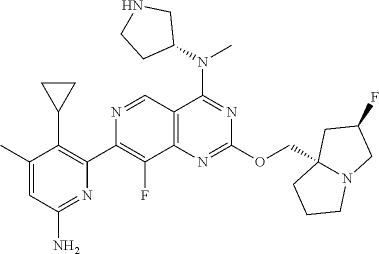 [SMILES: Cc1cc(N)nc(-c2ncc3c(N(C)[C@@H]4CCNC4)nc(OC[C@@]45CCCN4C[C@H](F)C5)nc3c2F)c1C1CC1]

### Step 5: 7-(6-amino-3-cyclopropyl-4-methylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and PMB group was prepared in a similar fashion to Example #71, Step 7. the residue was purified by reverse phase HPLC (column: Phenomenex luna C18 (250*70 mm,15 um); mobile phase: [water (trifluroacetic acid)-acetoniitrile]; B %: 8%-36%, 22 min) affording 7-(6-amino-3-cyclopropyl-4-methylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (80 mg, 11.77%) as a white solid. LCMS Rt=1.069 min, m/z=550.3 [M+H]⁺.

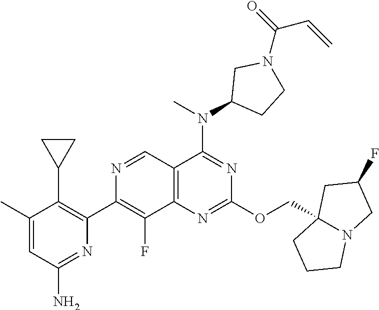 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1]

### Step 6: 1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 15%-55%, 8 min) affording 1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (7.61 mg, 17.27%) as a yellow oil. ¹H NMR (400 MHz, Acetonitrile-d3) δ 9.19-9.14 (m, 1H), 6.64-6.52 (m, 1H), 6.48-6.44 (m, 1H), 6.27-6.19 (m, 1H), 5.71-5.62 (m, 1H), 5.40-5.15 (m, 2H), 4.77-4.74 (m, 1H), 4.24-4.16 (m, 1H), 4.14-4.09 (m, 1H), 3.94 (dd, J=8.0, 12.9 Hz, 1H), 3.90-3.76 (m, 1H), 3.69-3.60 (m, 1H), 3.58-3.41 (m, 1H), 3.41-3.35 (m, 3H), 3.18-3.08 (m, 2H), 3.06 (s, 1H), 2.93-2.84 (m, 1H), 2.39 (s, 3H), 2.36 (br d, J=8.8 Hz, 1H), 2.31-2.25 (m, 1H), 2.19-2.16 (m, 1H), 2.11-1.99 (m, 3H), 1.91-1.82 (m, 3H), 1.74-1.65 (m, 1H), 0.56-0.47 (m, 2H), 0.03-0.06 (m, 2H). LCMS Rt=2.615 min, m/z=604.3 [M+H]⁺.

LCMS (5 to 95% acetonitrile in water+0.03% ammonium bicarbonate over 6 min) retention time 2.615 min, ESI+ found [M+H]=604.3.

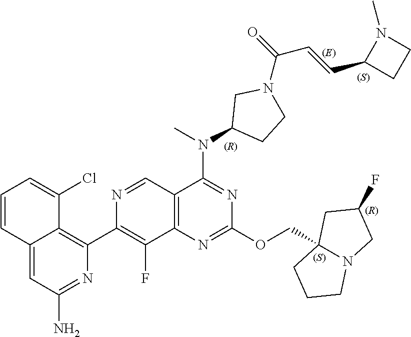

### Example 6: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one

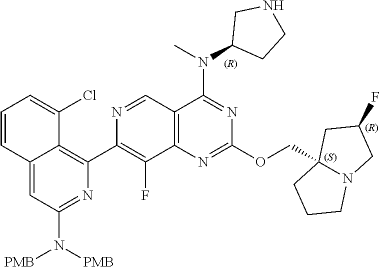

### Step 1: 7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The de-Boc protecting reaction was prepared in a similar fashion to Example #2, Step 4. The mixture was concentrated to dryness in vacuo affording 7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (623 mg, crude, hydrochloride salt) as a yellow solid used in next step without further purification. LCMS Rt=0.702 min, m/z=820.3 [M+H]⁺.

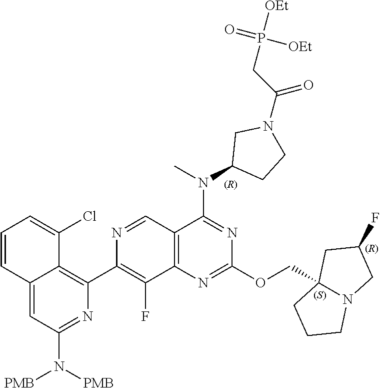

### Step 2: diethyl (2-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate

The amide coupling reaction was prepared in a similar fashion to Example #2, Step 5. The residue was purified by column chromatography (silica gel, 100-200 mesh, 0-15% methanol in dichloromethane) affording diethyl (2-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate (580 mg, 77.66%) as an orange gum. LCMS Rt=0.777 min, m/z=998.4 [M+H]⁺.

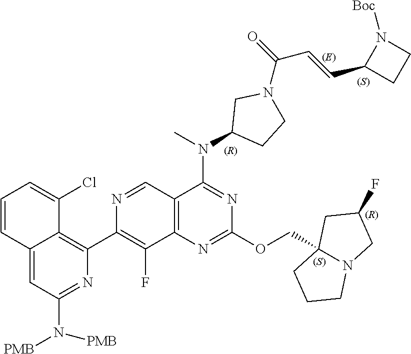

### Step 3: (S)-tert-butyl 2-((E)-3-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

The Horner-Wadsworth-Emmons reaction was prepared in a similar fashion to Example #2, Step 6. The mixture was concentrated to dryness in vacuo affording (S)-tert-butyl 2-((E)-3-((R)-3-((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (150 mg, crude) as an orange gum used in next step without further purification. LCMS Rt=0.801 min, m/z=1029.5 [M+H]⁺.

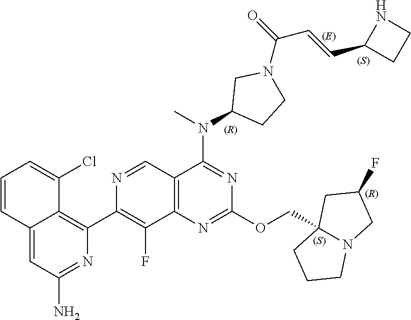

### Step 4: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one

The de-Boc and PMB protecting reaction was prepared in a similar fashion to Example #2, Step 7. The residue was purified by reverse phase HPLC (column: Phenomenex Luna C18 150*30 mm*5 um; mobile phase: [water (trifluoroacetic acid)-acetonitrile]; B %: 10%-35%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one (40 mg, 34.11%, trifluoroacetic salt) as a black solid. LCMS Rt=0.567 min, m/z=689.3 [M+H]⁺.

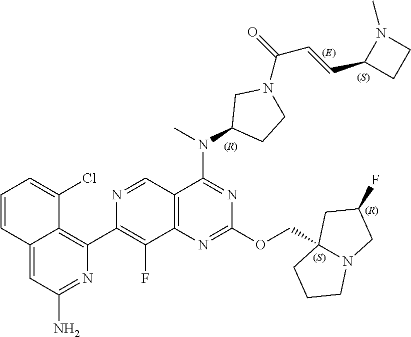

### Step 5: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one

The reductive amination reaction was prepared in a similar fashion to Example #2, Step 8. The residue was purified with reverse phase HPLC (column: Waters Xbridge Prep OBD C18 150*40 mm*10 um; mobile phase: [water (NH4HCO3)-acetonitrile]; B %: 25%-55%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-1-methylazetidin-2-yl)prop-2-en-1-one (2.1 mg, 7.99%) as a yellow amorphous solid: ¹H NMR (400 MHz, Acetonitrile-d3) δ 9.23-9.12 (m, 1H), 7.68-7.58 (m, 1H), 7.46-7.35 (m, 1H), 7.27-7.20 (m, 1H), 6.95-6.90 (m, 1H), 6.81-6.71 (m, 1H), 6.42-6.31 (m, 1H), 5.43-5.19 (m, 2H), 5.16 (br s, 2H), 4.24-4.17 (m, 1H), 4.16-4.09 (m, 1H), 4.08-3.75 (m, 2H), 3.69-3.60 (m, 1H), 3.58-3.43 (m, 2H), 3.42-3.38 (m, 3H), 3.32-3.23 (m, 1H), 3.19-3.08 (m, 2H), 3.08-3.04 (m, 1H), 2.94-2.85 (m, 1H), 2.84-2.75 (m, 1H), 2.41-2.28 (m, 2H), 2.23 (br d, J=9.4 Hz, 3H), 2.12-1.99 (m, 4H), 1.92-1.79 (m, 4H). LCMS Rt=2.677 min, m/z=703.3 [M+H]⁺.

LCMS (5 to 95% acetonitrile in water+0.1% ammonium bicarbonate over 6 mins) retention time 2.677 min, ESI+ found [M+H]=703.3.

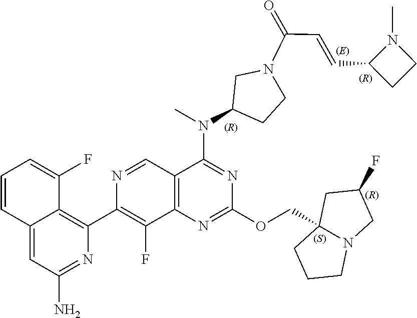

### Example 7: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one

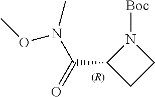

### Step 1: (R)-tert-butyl 2-(methoxy(methyl)carbamoyl)azetidine-1-carboxylate

The amide coupling reaction was prepared in a similar fashion to Example #2, Step 1. The residue was purified by column chromatography (silica gel, 100-200 mesh, 0-100% ethyl acetate in petroleum ether) affording (R)-tert-butyl 2-(methoxy(methyl)carbamoyl)azetidine-1-carboxylate (2.1 g, 85.62%) as a colorless solid: ¹HNMR (400 MHz, Chloroform-d) δ 5.10-4.95 (m, 1H), 4.09-3.99 (m, 1H), 3.92-3.81 (m, 1H), 3.70 (s, 3H), 3.30-3.17 (m, 3H), 2.46 (dtd, J=6.4, 9.0, 11.1 Hz, 1H), 2.19-2.06 (m, 1H), 1.42 (s, 9H). LCMS Rt=0.678 min, m/z=244.1 [M+H]⁺.

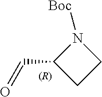

### Step 2: (R)-tert-butyl 2-formylazetidine-1-carboxylate

The reduction reaction was prepared in a similar fashion to Example #2, Step 2. The mixture was concentrated to dryness in vacuo affording (R)-tert-butyl 2-formylazetidine-1-carboxylate (220 mg, crude) as a colorless oil used in next step without further purification.

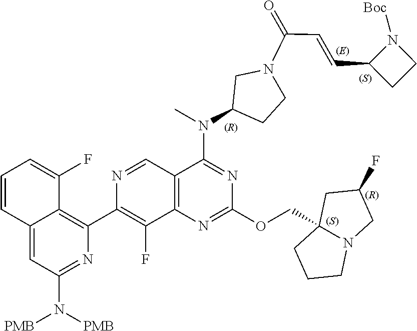

### Step 3: (S)-tert-butyl 2-((E)-3-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

The Horner-Wadsworth-Emmons reaction was prepared in a similar fashion to Example #2, Step 6. The residue was purified by column chromatography (silica gel, 100-200 mesh, 0-100% methanol in dichloromethane) affording (S)-tert-butyl 2-((E)-3-((R)-3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (90 mg, 58.16%) as a white solid. LCMS Rt=1.173 min, m/z=1013.5[M+H]⁺.

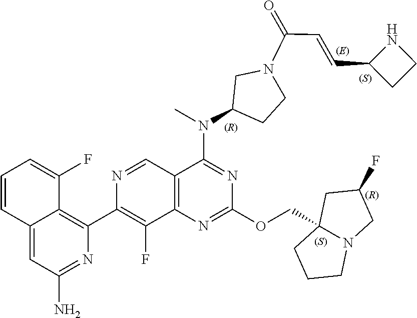

### Step 4: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one

The de-Boc and PMB protecting reaction was prepared in a similar fashion to Example #2, Step 7. The residue was purified by reverse phase HPLC: column: Phenomenex Luna 80*30 mm*3 um; mobile phase: [water (trifluroacetic acid)-acetoniitrile];B %:5%-35%, 8 min) affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one (20 mg, 32.18%, trifluoroacetic salt) as a yellow solid. LCMS Rt=0.626 min, m/z=673.3 [M+H]⁺.

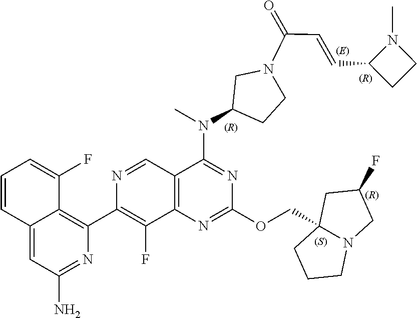

### Step 5: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one

The reductive amination reaction was prepared in a similar fashion to Example #2, Step 8. The residue was purified by reverse phase HPLC (column: Waters Xbridge Prep OBD C18 150*40 mm*10 um; mobile phase: [water (NH4HCO3)-acetonitrile]; B %: 20%-50%, 8 min) affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-1-methylazetidin-2-yl)prop-2-en-1-one (5.2 mg, 14.65%) as a yellow solid: ¹H NMR (400 MHz, Acetonitrile-d3) δ 9.22 (d, J=2.6 Hz, 1H), 7.55-7.44 (m, 2H), 6.93 (d, J=2.0 Hz, 1H), 6.89-6.74 (m, 2H), 6.45-6.35 (m, 1H), 5.40-5.32 (m, 1H), 5.24-5.15 (m, 2H), 4.29-4.21 (m, 1H), 4.17 (br d, J=2.3 Hz, 1H), 4.15-4.04 (m, 1H), 3.99-3.89 (m, 1H), 3.88-3.76 (m, 1H), 3.71-3.47 (m, 3H), 3.44 (d, J=1.9 Hz, 3H), 3.35-3.27 (m, 1H), 3.22-3.14 (m, 2H), 3.13-3.07 (m, 1H), 2.96-2.78 (m, 2H), 2.44-2.29 (m, 3H), 2.26 (d, J=7.9 Hz, 3H), 2.14 (br s, 2H), 2.11-2.06 (m, 2H), 1.93-1.78 (m, 3H). LCMS Rt=1.768 min, m/z=687.3 [M+H]⁺.

LCMS (5 to 95% acetonitrile in water+0.1% ammonium bicarbonate over 6 mins) retention time 1.768 min, ESI+ found [M+H]=687.3.

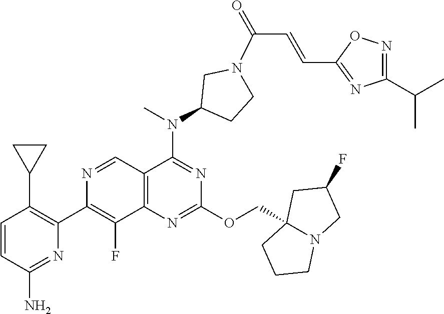 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1]

### Example 8: (E)-1-((R)-3-((7-(6-amino-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

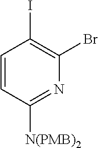

### Step 1: 6-bromo-5-iodo-N,N-bis(4-methoxybenzyl)pyridin-2-amine

The PMB protection reaction was prepared in a similar fashion to Example #71, Step 1. The crude product was purified by column chromatography (silica gel, 100-200 mesh, 0-10% ethyl acetate in petroleum ether) affording 6-bromo-5-iodo-N,N-bis(4-methoxybenzyl)pyridin-2-amine (2.5 g, 78.45%) as a yellow oil. LCMS Rt=1.037 min, m/z=538.0 [M+H]⁺.

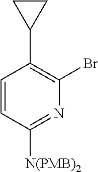

### Step 2: 6-bromo-5-cyclopropyl-N,N-bis(4-methoxybenzyl)pyridin-2-amine

To a solution of 6-bromo-5-iodo-N,N-bis(4-methoxybenzyl)pyridin-2-amine (2.5 g, 4.64 mmol) cyclopropylboronic acid (406.23 mg, 4.73 mmol) cesium carbonate (4.53 g, 13.91 mmol) in dioxane (10 mL) water (1 mL) was added cyclopentyl(diphenyl)phosphane; dichloropalladium;iron (678.51 mg, 927.29 umol), the mixture was stirred at 100° C. for 3 h under nitrogen atmosphere, the reaction mixture was diluted with water (10 mL) and extracted with ethyl acetate (2×60 mL). The combined organic layers were dried over sodium sulphate and concentrated under vacuo, the resulting residue was purified by reverse phase HPLC (column: Welch Xtimate C18 250*70 mm #10 um; mobile phase: [water (NH4HCO3)-acetonitrile]; B %: 65%-95%, 17 min) affording 6-bromo-5-cyclopropyl-N,N-bis(4-methoxybenzyl)pyridin-2-amine (800 mg, 38.06%) as a yellow oil. LCMS Rt=3.036 min, m/z=452.1 [M+H]⁺.

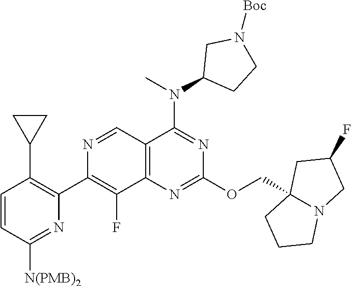

### Step 3: (R)-tert-butyl 3-((7-(6-(bis(4-methoxybenzyl)amino)-3-cyclopropylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The stile reaction was prepared in a similar fashion to Example #71, Step 6. The residue was purified by column chromatography (silica gel, 100-200 mesh, 95-100% ethyl acetate in petroleum ether) affording tert-butyl (3R)-3-[[7-[6-[bis[(4-methoxyphenyl)methyl]amino]-3-cyclopropyl-2-pyridyl]-8-fluoro-2-[[(2R,8S)-2-fluoro-1,2,3,5,6,7-hexahydropyrrolizin-8-yl]methoxy]pyrido[4,3-d]pyrimidin-4-yl]-methyl-amino]pyrrolidine-1-carboxylate (350 mg, 24.07%) as a yellow oil. LCMS Rt=0.757 min, m/z=876.5 [M+H]⁺.

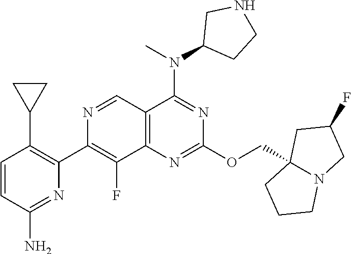 [SMILES: CN(c1nc(OC[C@@]23CCCN2C[C@H](F)C3)nc2c(F)c(-c3nc(N)ccc3C3CC3)ncc12)[C@@H]1CCNC1]

### Step 4: 7-(6-amino-3-cyclopropylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and PMB was prepared in a similar fashion to Example #71, Step 7. The residue was purified by reverse phase HPLC (column: Phenomenex Luna 80*30 mm*3 um; mobile phase: [water (trifluroacetic acid)-acetoniitrile]; B %: 1%-35%, 8 min) affording 7-(6-amino-3-cyclopropylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (180 mg, 98.06%, trifluoroacetate salt) as a yellow oil. LCMS Rt=0.382 min, m/z=536.3 [M+H]⁺.

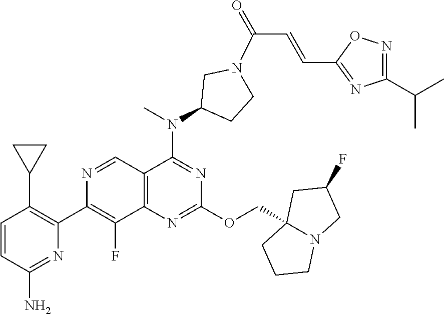 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1]

### Step 5: (E)-1-((R)-3-((7-(6-amino-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge Prep OBD C18 150*40 mm*10 um; mobile phase: [water (NH4HCO3)-acetonitrile]; B %: 35%-55%, 8 min) affording (E)-1-[(3R)-3-[[7-(6-amino-3-cyclopropyl-2-pyridyl)-8-fluoro-2-[[(2R,8S)-2-fluoro-1,2,3,5,6,7-hexahydropyrrolizin-8-yl]methoxy]pyrido[4,3-d]pyrimidin-4-yl]-methyl-amino]pyrrolidin-1-yl]-3-(3-isopropyl-1,2,4-oxadiazol-5-yl)prop-2-en-1-one (34.15 mg, 39.63%) as a yellow amorphous solid: ¹H NMR (400 MHz, Acetonitrile-d3) δ 9.19 (d, J=1.6 Hz, 1H), 7.49-7.33 (m, 2H), 7.18 (d, J=8.5 Hz, 1H), 6.56 (d, J=8.5 Hz, 1H), 5.43-5.16 (m, 2H), 4.85 (s, 2H), 4.25-4.10 (m, 3H), 4.04-3.96 (m, 1H), 3.91-3.70 (m, 2H), 3.63-3.47 (m, 1H), 3.41 (s, 3H), 3.15-3.06 (m, 3H), 2.88 (quin, J=7.6 Hz, 1H), 2.42-2.28 (m, 2H), 2.12-1.97 (m, 3H), 1.91-1.80 (m, 3H), 1.71-1.63 (m, 1H), 1.32 (dd, J=7.0, 8.6 Hz, 6H), 0.70-0.60 (m, 2H), 0.52-0.42 (m, 2H). LCMS Rt=2.707 min, m/z=700.3 [M+H]⁺.

LCMS (5 to 95% acetonitrile in water+0.03% ammonium bicarbonate over 6 mins) retention time 2.707 min, ESI+ found [M+H]=700.3.

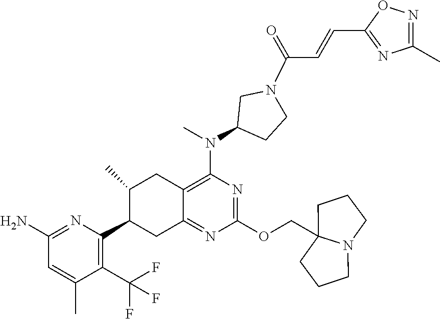 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1]

### Example 9: (E)-1-((R)-3-(((6R,7R)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

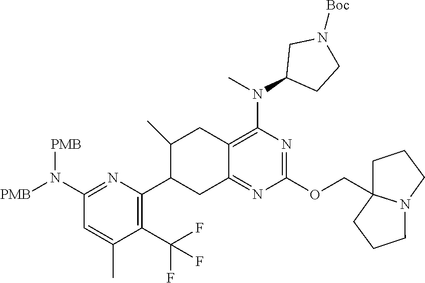

### Step 1: (3R)-tert-butyl 3-((7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate

The substitution reaction was prepared in a similar fashion to Example #65, Step 14. The resulting residue was purified by column chromatography (silica gel, 100-200 mesh, 0-100% ethyl acetate in petroleum ether) affording (3R)-tert-butyl 3-((7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate (300 mg, 50.85%) as a white solid. LCMS Rt=0.674 min, m/z=899.5 [M+H]⁺.

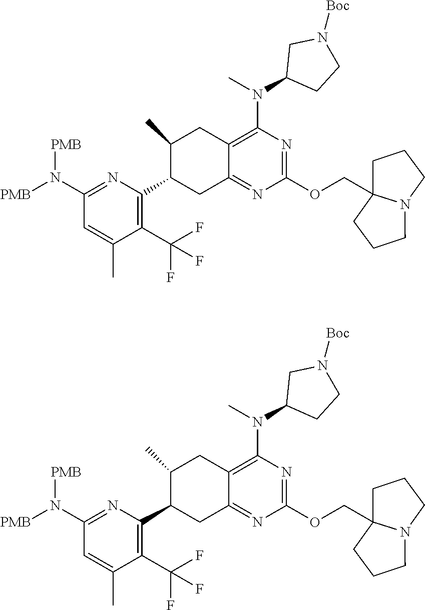

### Step 2: (R)-tert-butyl 3-(((6S,7S)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate and (R)-tert-butyl 3-(((6R,7R)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate

The mixture of diastereoisomers was separated by SFC to give arbitrarily assigned: (R)-tert-butyl 3-(((6S,7S)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate (Peak 1, retention time=1.948 min) (150 mg, 25.42%) as a white solid. LCMS Rt=0.674 min, m/z=899.5 [M+H]⁺. (R)-tert-butyl 3-(((6R,7R)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidine-1-carboxylate (Peak 2, retention time=2.865 min) (120 mg, 20.34%) as a white solid. LCMS Rt=0.674 min, m/z=899.5 [M+H]⁺. SFC (column: REGIS(S,S)WHELK-O1 (250 mm*25 mm,10 um); mobile phase: [0.1% NH₃H₂O IPA]; B %: 62%-62%, 20 min).

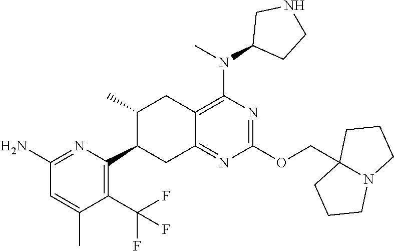 [SMILES: Cc1cc(N)nc([C@@H]2Cc3nc(OCC45CCCN4CCC5)nc(N(C)[C@@H]4CCNC4)c3C[C@H]2C)c1C(F)(F)F]

### Step 3: (6R,7R)-7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine

The deprotection of Boc and PMB was prepared in a similar fashion to Example #65, Step 15. The reaction mixture was concentrated in vacuo and purified by reverse phase HPLC (column: Phenomenex Luna 80*30 mm*3 um; mobile phase: [water (trifluroacetic acid)-acetoniitrile]; B %: 1%-30%, 8 min) affording (6R,7R)-7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine (80 mg, 89.07%, trifluoroacetate salt) as a white solid: LCMS Rt=0.578 min, m/z=559.3 [M+H]⁺.

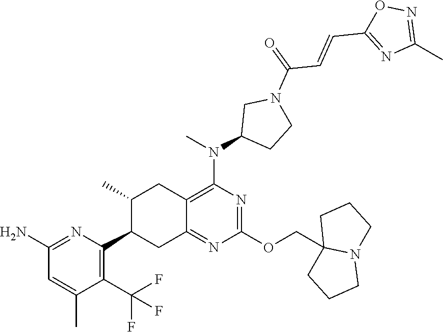 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1]

### Step 4: (E)-1-((R)-3-(((6R,7R)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #65, Step 16. The crude product was purified by reverse phase HPLC (column: Waters Xbridge Prep OBD C18 150*40 mm*10 um; mobile phase: [water (NH4HCO3)-acetonitrile]; B %: 30%-70%, 8 min) affording (E)-1-((R)-3-(((6R,7R)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (12.77 mg, 24.73%) as a yellow solid: 1H NMR (400 MHz, Acetonitrile-d3) δ 7.51-7.43 (m, 1H), 7.41-7.33 (m, 1H), 6.28 (s, 1H), 5.24 (s, 2H), 4.79-4.58 (m, 1H), 4.16 (dd, J=8.0, 10.3 Hz, 1H), 4.03-3.89 (m, 3H), 3.87-3.74 (m, 1H), 3.68 (br d, J=8.6 Hz, 1H), 3.52-3.36 (m, 1H), 3.17 (td, J=5.3, 10.3 Hz, 1H), 3.07-2.94 (m, 3H), 2.93 (d, J=3.1 Hz, 3H), 2.89-2.81 (m, 1H), 2.66 (s, 4H), 2.41 (s, 1H), 2.40 (s, 1H), 2.38 (d, J=3.5 Hz, 2H), 2.30-2.23 (m, 1H), 2.21-2.14 (m, 1H), 2.13-2.03 (m, 2H), 1.91 (td, J=6.1, 12.1 Hz, 2H), 1.86-1.71 (m, 4H), 1.66-1.54 (m, 2H), 0.77 (d, J=6.3 Hz, 3H). LCMS Rt=2.987 min, m/z=695.4 [M+H]⁺.

LCMS (5 to 95% acetonitrile in water+0.03% ammonium bicarbonate over 6 mins) retention time 2.987 min, ESI+ found [M+H]=695.4.

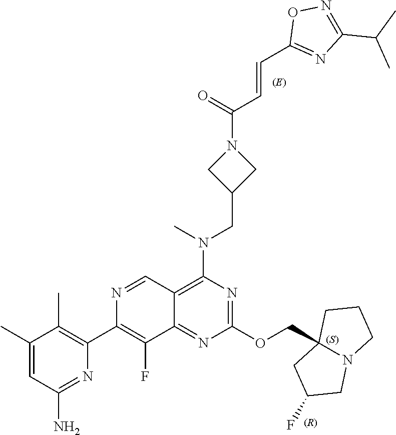

### Example 10: (E)-1-(3-(((7-(6-amino-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

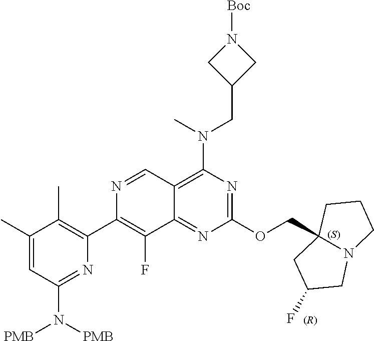

### Step 1: tert-butyl 3-(((7-(6-(bis(4-methoxybenzyl)amino)-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The residue was purified by column chromatography (silica gel, 100-200 mesh, 0-100% ethyl acetate in petroleum ether) affording tert-butyl 3-(((7-(6-(bis(4-methoxybenzyl)amino)-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (1.09 g, 49.01%) as a brown oil. LCMS Rt=2.282 min, m/z=864.5 [M+H]⁺.

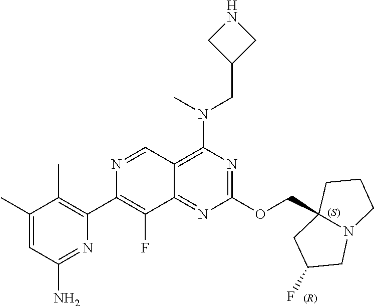

### Step 2: 7-(6-amino-3,4-dimethylpyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The crude product was purified by reverse phase HPLC (column: Phenomenex Luna C18 150*30 mm*5 um; mobile phase: [water (trifluroacetic acid)-acetoniitrile]; B %: 1%-20%, 8 min) affording 7-(6-amino-3,4-dimethylpyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (60 mg, 40.71%, trifluoroacetate salt) as a pale brown oil. LCMS Rt=0.376 min, m/z=524.3 [M+H]⁺.

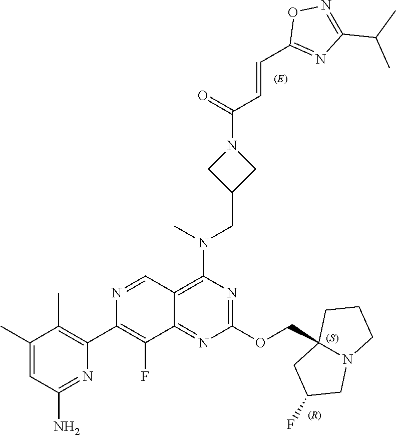

### Step 3: (E)-1-(3-(((7-(6-amino-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

The amide coupling reaction was prepared in a similar fashion to Example #71, Step 11. The reaction mixture was purified by reverse phase HPLC (column: Waters Xbridge Prep OBD C18 150*40 mm*10 um; mobile phase: [water (NH4HCO3)-acetonitrile]; B %: 30%-60%, 8 min) affording (E)-1-(3-(((7-(6-amino-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one (1.7 mg, 2.68%) as a yellow oil: ¹H NMR (400 MHz, Acetonitrile-d3) δ 9.27-9.19 (m, 1H), 7.34-7.26 (m, 1H), 7.17-7.08 (m, 1H), 6.52-6.45 (m, 1H), 5.37-5.17 (m, 1H), 4.82-4.59 (m, 2H), 4.46 (br t, J=8.7 Hz, 1H), 4.29-4.21 (m, 2H), 4.21-4.10 (m, 4H), 3.95-3.88 (m, 1H), 3.56 (s, 3H), 3.28-3.17 (m, 3H), 3.15-3.07 (m, 2H), 2.97 (br s, 1H), 2.25 (s, 6H), 1.91-1.73 (m, 4H), 1.62-1.44 (m, 2H), 1.31 (d, J=7.0 Hz, 6H). LCMS Rt=1.901 min, m/z=688.3 [M+H]⁺.

LCMS (5 to 95% acetonitrile in water+0.1% trifluoroacetic acid over 6 mins) retention time 1.901 min, ESI+ found [M+H]=688.3.

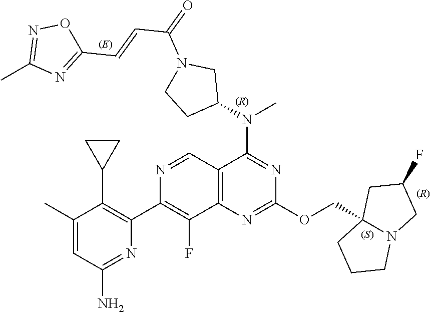

### Example 11: (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

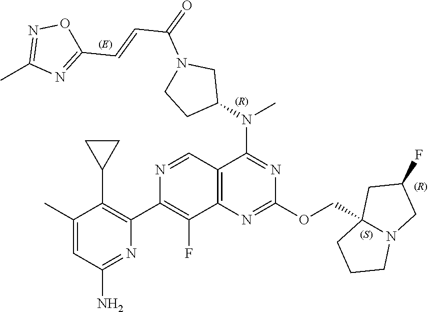

### Step 1: (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #71, Step 11. The residue was purified by reverse phase HPLC (column: Waters Xbridge BEH C18 100*30 mm*10 um; mobile phase: [water (NH4HCO3)-acetonitrile]; B %: 35%-65%, 8 min) affording (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one (32.23 mg, 14.80%) as a yellow solid. ¹H NMR (400 MHz, Acetonitrile-d3) δ 9.20-9.14 (m, 1H), 7.50-7.32 (m, 2H), 6.48-6.43 (m, 1H), 5.18 (br d, J=2.6 Hz, 2H), 4.79-4.71 (m, 2H), 4.22-4.18 (m, 1H), 4.16 (s, 1H), 4.04-3.96 (m, 1H), 3.91-3.82 (m, 1H), 3.80-3.70 (m, 1H), 3.64-3.45 (m, 1H), 3.41 (s, 3H), 3.19-3.08 (m, 2H), 3.07-3.02 (m, 1H), 2.93-2.82 (m, 1H), 2.44-2.40 (m, 1H), 2.39 (s, 4H), 2.37 (s, 2H), 2.35-2.27 (m, 1H), 2.22 (br d, J=4.4 Hz, 1H), 2.10 (br d, J=2.8 Hz, 1H), 2.07-1.99 (m, 1H), 1.91-1.78 (m, 3H), 1.74-1.64 (m, 1H), 0.57-0.48 (m, 2H), −0.01 (br d, J=4.5 Hz, 2H). LCMS Rt=2.723 min, m/z=686.3 [M+H]⁺.

LCMS (5 to 95% acetonitrile in water+0.03% ammonium bicarbonate over 6 min) retention time 2.723 min, ESI+ found [M+H]=686.3.

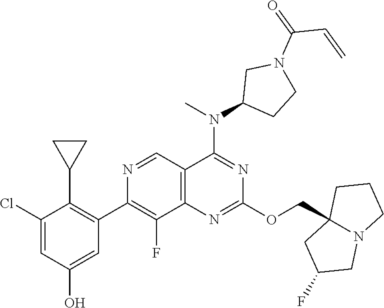 [SMILES: C=CC(=O)N1CC[C@@H](N(C)c2nc(OC[C@@]34CCCN3C[C@H](F)C4)nc3c(F)c(-c4cc(O)cc(Cl)c4C4CC4)ncc23)C1]

### Example 12: 1-((R)-3-((7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

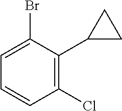 [SMILES: Clc1cccc(Br)c1C1CC1]

### Step 1: 1-bromo-3-chloro-2-cyclopropyl-benzene

To a solution of 1-bromo-3-chloro-2-iodo-benzene (20 g, 63.02 mmol) in dioxane (180 mL) and water (60 mL) were added cyclopropylboronic acid (7.04 g, 81.93 mmol), potassium phosphate (48.16 g, 226.88 mmol) and Palladium 5% on bariumsulfate (2.31 g, 3.15 mmol), then the mixture was heated to 100° C. and stirred for 16 h under nitrogen atmosphere. The mixture was diluted with water (80 mL), extracted with ethyl acetate (2×200 mL). The combined organic layers were dried over sodium sulphate and concentrated under vacuo. The crude was purified by reverse phase HPLC (column: Welch Xtimate C18 250*70 mm #10 um; mobile phase: [water (NH4HCO3)-acetonitrile]; B %: 70%-96%, 20 min) affording 1-bromo-3-chloro-2-cyclopropyl-benzene (6 g, 41.12%) as a colorless oil.

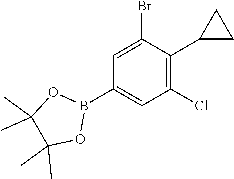 [SMILES: CC1(C)OB(c2cc(Cl)c(C3CC3)c(Br)c2)OC1(C)C]

### Step 2: 2-(3-bromo-5-chloro-4-cyclopropyl-phenyl)-4,4,5,5-tetramethyl-1,3,2-dioxaborolane

To a solution of 1-bromo-3-chloro-2-cyclopropyl-benzene (1 g, 4.32 mmol) in hexane (15 mL) were added 4,4,5,5-tetramethyl-1,3,2-dioxaborolane (1.66 g, 12.96 mmol, 1.88 mL), (1Z,5Z)-cycloocta-1,5-diene;2,4-dimethyl-BLAHbicyclo[1.1.0]butane (143.16 mg, 215.97 umol) and 4-tert-butyl-2-(4-tert-butyl-2-pyridyl)pyridine (69.56 mg, 259.16 umol), then the mixture was heated to 60° C. and stirred for 2 h under nitrogen atmosphere. The mixture was diluted with water (10 mL), extracted with ethyl acetate (2×20 mL). The combined organic layers were concentrated to dryness in vacuo and purified by column chromatography (silica gel, 100-200 mesh, 0-5% ethyl acetate in petroleum ether) affording 2-(3-bromo-5-chloro-4-cyclopropyl-phenyl)-4,4,5,5-tetramethyl-1,3,2-dioxaborolane (1 g, 64.76%) as a yellow oil.

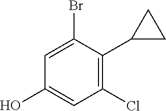 [SMILES: Oc1cc(Cl)c(C2CC2)c(Br)c1]

### Step 3: 3-bromo-5-chloro-4-cyclopropyl-phenol

To a solution of 2-(3-bromo-5-chloro-4-cyclopropyl-phenyl)-4,4,5,5-tetramethyl-1,3,2-dioxaborolane (1 g, 2.80 mmol) in tetrahydrofuran (10 mL) and water (5 mL) were added acetic acid (10.78 g, 179.59 mmol) and hydrogen peroxide (1.8 g, 53.43 mmol) at 0° C., the reaction was stirred at 0° C. for 1 h. The mixture was quenched by saturated sodium sulfite (50 mL) at 0° C., extracted with ethyl acetate (3×100 ml). The combined organic layers were concentrated to dryness in vacuo and purified by column chromatography (silica gel, 100-200 mesh, 0-10% ethyl acetate in petroleum ether) affording 3-bromo-5-chloro-4-cyclopropyl-phenol (660 mg, 95.32%) as a colorless oil.

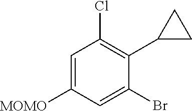

### Step 4: 1-bromo-3-chloro-2-cyclopropyl-5-(methoxymethyl)benzene

To a solution of 3-bromo-5-chloro-4-cyclopropyl-phenol (660 mg, 2.67 mmol) in dichloromethane (7 mL) were added N, N-diisopropylethylamine (1.03 g, 8.00 mmol) and chloromethyl methyl ether (429.38 mg, 5.33 mmol) at 0° C., then the mixture was warmed to 20° C. and stirred for 1 h. The mixture was diluted with water (20 mL), extracted with ethyl acetate (2×20 mL). The combined organic layers were concentrated to dryness in vacuo and purified by column chromatography (silica gel, 100-200 mesh, 0-4% ethyl acetate in petroleum ether) affording 1-bromo-3-chloro-2-cyclopropyl-5-(methoxymethyl)benzene (600 mg, 77.17%) as a colorless oil.

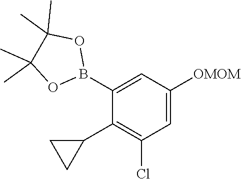

### Step 5: 2-[3-chloro-2-cyclopropyl-5-(methoxymethoxy)phenyl]-4,4,5,5-tetramethyl-1,3,2-dioxaborolane

To a solution of 1-bromo-3-chloro-2-cyclopropyl-5-(methoxymethoxy)benzene (400 mg, 1.37 mmol) in dioxane (5 mL) were added 4,4,5,5-tetramethyl-2-(4,4,5,5-tetramethyl-1,3,2-dioxaborolan-2-yl)-1,3,2-dioxaborolane (696.75 mg, 2.74 mmol), potassium acetate (403.92 mg, 4.12 mmol) and (1,1′-bis(diphenylphosphino)ferrocene)palladium(II) dichloride (100.38 mg, 137.19 umol), then the mixture was heated to 100° C. and stirred for 12 h under nitrogen atmosphere. The mixture was diluted with water (20 mL), extracted with ethyl acetate (2×20 mL). The combined organic layers were concentrated to dryness in vacuo and purified by column chromatography (silica gel, 100-200 mesh, 0-5% ethyl acetate in petroleum ether) affording 2-[3-chloro-2-cyclopropyl-5-(methoxymethoxy)phenyl]-4,4,5,5-tetramethyl-1,3,2-dioxaborolane (260 mg, 51.49%) as a pale green oil.

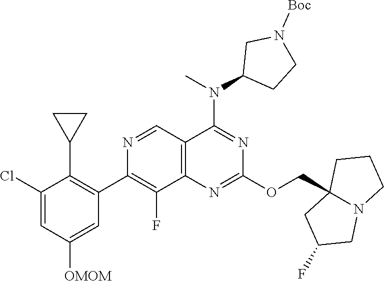

### Step 6: (R)-tert-butyl 3-((7-(3-chloro-2-cyclopropyl-5-(methoxymethoxy)phenyl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

To a solution of (R)-tert-butyl 3-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (200 mg, 371.05 umol) in dioxane (3 mL) and water (1.5 mL) were added 2-[3-chloro-2-cyclopropyl-5-(methoxymethoxy)phenyl]-4,4,5,5-tetramethyl-1,3,2-dioxaborolane (251.30 mg, 742.09 umol), potassium phosphate (236.28 mg, 1.11 mmol) and methanesulfonic acid(2-dicyclohexylphosphino-2′,4′,6′-triisopropyl-1,1′-biphenyl) [2-(2′-amino-1,1′-biphenyl)]palladium(II) (31.41 mg, 37.10 umol), then the mixture was heated to 60° C. and stirred for 8 h under nitrogen atmosphere. The mixture was diluted with water (20 mL), extracted with dichloromethane (2×20 mL). The combined organic layers were concentrated to dryness in vacuo and purified by column chromatography (silica gel, 100-200 mesh, 0-12% methanol in dichloromethane) affording (R)-tert-butyl 3-((7-(3-chloro-2-cyclopropyl-5-(methoxymethoxy)phenyl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (250 mg, 65.00%) as a pale yellow oil. LCMS Rt=0.738 min, m/z=714.3 [M+H]⁺.

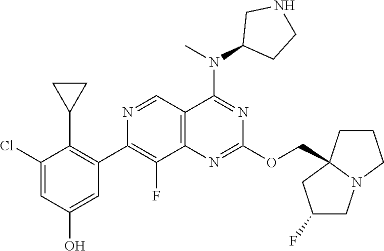 [SMILES: CN(c1nc(OC[C@@]23CCCN2C[C@H](F)C3)nc2c(F)c(-c3cc(O)cc(Cl)c3C3CC3)ncc12)[C@@H]1CCNC1]

### Step 7: 7-(3-chloro-2-cyclopropyl-5-(methoxymethoxy)phenyl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and MOM group was prepared in a similar fashion to Example #71, Step 7. The reaction mixture was concentrated in vacuo affording 7-(3-chloro-2-cyclopropyl-5-(methoxymethoxy)phenyl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (85 mg, crude, hydrochloric acid salt) used in next step without further purification. LCMS Rt=0.591 min, m/z=614.3 [M+H]⁺.

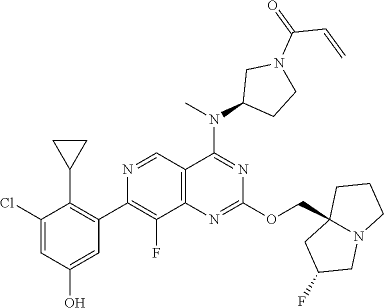 [SMILES: C=CC(=O)N1CC[C@@H](N(C)c2nc(OC[C@@]34CCCN3C[C@H](F)C4)nc3c(F)c(-c4cc(O)cc(Cl)c4C4CC4)ncc23)C1]

### Step 8: 1-((R)-3-((7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC(column: Waters Xbridge Prep OBD C18 150*40 mm*10 um; mobile phase: [water (NH4HCO3)-acetonitrile]; B %: 30%-60%, 8 min) affording 1-((R)-3-((7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (32.68 mg, 36.16%) as a yellow solid: ¹H NMR (400 MHz, Acetonitrile-d3) δ 9.21-9.09 (m, 1H), 7.00-6.92 (m, 1H), 6.86-6.76 (m, 1H), 6.66-6.47 (m, 1H), 6.29-6.18 (m, 1H), 5.72-5.61 (m, 1H), 5.18 (br s, 2H), 4.22-4.16 (m, 1H), 4.15-4.09 (m, 1H), 4.08-3.90 (m, 1H), 3.89-3.76 (m, 1H), 3.69-3.59 (m, 1H), 3.57-3.42 (m, 1H), 3.38 (d, J=2.3 Hz, 3H), 3.22-3.08 (m, 2H), 3.06 (s, 1H), 2.96-2.82 (m, 1H), 2.40-2.13 (m, 4H), 2.06-2.01 (m, 1H), 1.91-1.77 (m, 4H), 0.68-0.49 (m, 2H), 0.12-0.09 (m, 2H). LCMS Rt=2.843 min, m/z=624.2 [M+H]⁺.

LCMS (5 to 95% acetonitrile in water+0.1% ammonium bicarbonate over 6 mins) retention time 2.843 min, ESI+ found [M+H]=624.2.

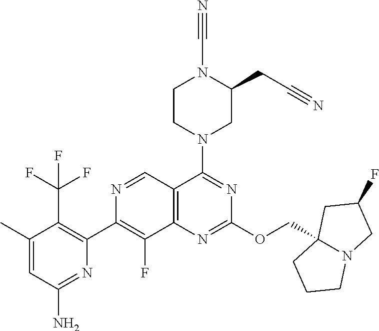 [SMILES: Cc1cc(N)nc(-c2ncc3c(N4CCN(C#N)[C@@H](CC#N)C4)nc(OC[C@@]45CCCN4C[C@H](F)C5)nc3c2F)c1C(F)(F)F]

### Example 13: (S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carbonitrile

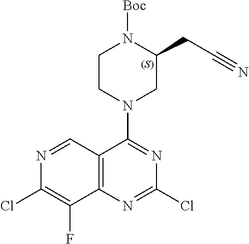

### Step 1: (S)-tert-butyl 2-(cyanomethyl)-4-(2,7-dichloro-8-fluoropyrido[4,3-d]pyrimidin-4-yl)piperazine-1-carboxylate

The substitution reaction was prepared in a similar fashion to Example #71, Step 3. The mixture was concentrated in vacuo affording (S)-tert-butyl 2-(cyanomethyl)-4-(2,7-dichloro-8-fluoropyrido[4,3-d]pyrimidin-4-yl)piperazine-1-carboxylate (1.5 g, crude) as a brown solid, used in the next step without further purification. LCMS Rt=0.681 min, m/z=441.1 [M+H]⁺.

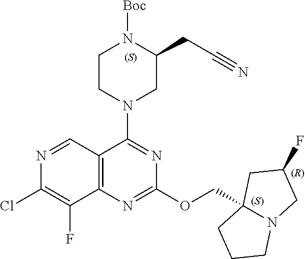

### Step 2: (S)-tert-butyl 4-(7-chloro-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate

The substitution reaction was prepared in a similar fashion to Example #71, Step 4. The mixture was purified by reverse phase HPLC(column: Welch Xtimate C18 250*70 mm #10 um; mobile phase: [water (NH4HCO3)-acetonitrile]; B %: 40%-70%, 20 min) affording (S)-tert-butyl 4-(7-chloro-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate (1.1 g, 44.00%) as a white solid. LCMS Rt=2.111 min, m/z=563.2 [M+H]⁺.

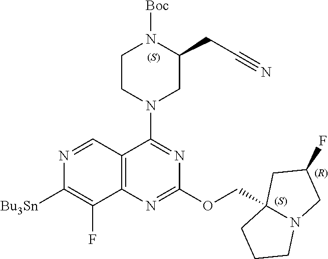

### Step 3: (S)-tert-butyl 2-(cyanomethyl)-4-(8-fluoro-2-(((2R,7aS)-2-fluorohexahydro-1H-pyrrolizin-7a-yl)methoxy)-7-(tributylstannyl)pyrido[4,3-d]pyrimidin-4-yl)piperazine-1-carboxylate

The tin reagent formation was prepared in a similar fashion to Example #71, Step 5. The mixture was purified by column chromatography (silica gel, 100-200 mesh, 80-100% ethyl acetate in petroleum ether) affording (S)-tert-butyl 2-(cyanomethyl)-4-(8-fluoro-2-(((2R,7aS)-2-fluorohexahydro-1H-pyrrolizin-7a-yl)methoxy)-7-(tributylstannyl)pyrido[4,3-d]pyrimidin-4-yl)piperazine-1-carboxylate (480 mg, 52.16%) as a yellow oil. LCMS Rt=0.673 min, m/z=819.4 [M+H]⁺.

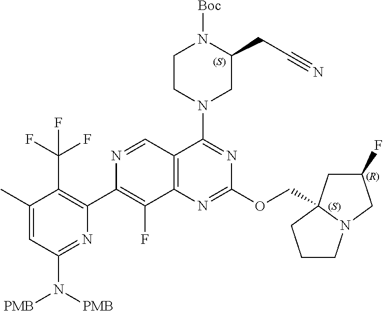

### Step 4: (S)-tert-butyl 4-(7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate

The Stille reaction was prepared in a similar fashion to Example #71, Step 6. The mixture was concentrated to dryness in vacuo affording (S)-tert-butyl 4-(7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carboxylate (500 mg, crude) as a yellow oil, used in next step without any further purification. LCMS Rt=0.858 min, m/z=943.4 [M+H]⁺.

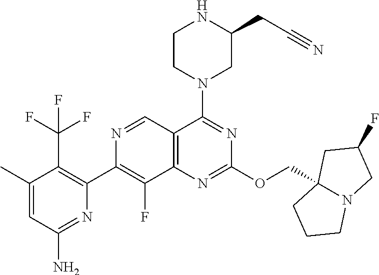 [SMILES: Cc1cc(N)nc(-c2ncc3c(N4CCN[C@@H](CC#N)C4)nc(OC[C@@]45CCCN4C[C@H](F)C5)nc3c2F)c1C(F)(F)F]

### Step 5: 2-((S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile

The deprotection of Boc and PMB was prepared in a similar fashion to Example #71, Step 7. The crude product was purified by reverse phase HPLC (column: Phenomenex Luna 80*30 mm*3 um; mobile phase: [column: Phenomenex Luna 80*30 mm*3 um; mobile phase: [water (trifluroacetic acid)-acetoniitrile]; B %: 1%-30%, 8 min) affording 2-((S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (60 mg, 15.79%, trifluoroacetate salt) as a white solid. LCMS Rt=0.547 min, m/z=603.3 [M+H]⁺.

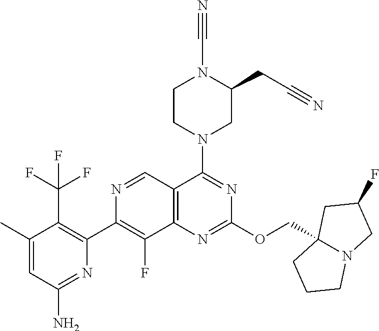 [SMILES: Cc1cc(N)nc(-c2ncc3c(N4CCN(C#N)[C@@H](CC#N)C4)nc(OC[C@@]45CCCN4C[C@H](F)C5)nc3c2F)c1C(F)(F)F]

### Step 6: (S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carbonitrile

To a solution of 2-((S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazin-2-yl)acetonitrile (30 mg, 41.81 umol) in acetonitrile (2 mL) was added potassium carbonate (17.33 mg, 125.42 umol) and bromine cyanide (6.64 mg, 62.71 umol), then the mixture was stirred at 50° C. for 60 min. The reaction mixture was quenched with saturated sodium carbonate (10 mL) and extracted with dichlormethane (2×10 mL). The combined organic layers were dried over sodium sulphate and concentrated in vacuo. The crude product was purified by reverse phase HPLC (column: Waters Xbridge BEH C18 100*30 mm*10 um; mobile phase: [water (NH4HCO3)-acetonitrile]; B %: 30%-55%, 8 min) affording (S)-4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)-2-(cyanomethyl)piperazine-1-carbonitrile (1.70 mg, 6.20%) as a white solid: ¹H NMR (400 MHz, Dimethylsulfoxide-d6) δ 9.07 (s, 1H), 6.81 (s, 2H), 6.51 (s, 1H), 5.43-5.15 (m, 1H), 4.24-4.10 (m, 3H), 4.09-4.03 (m, 1H), 3.98 (br d, J=2.9 Hz, 1H), 3.90-3.78 (m, 2H), 3.76-3.68 (m, 1H), 3.59-3.51 (m, 1H), 3.22-3.06 (m, 4H), 3.02 (s, 1H), 2.87-2.78 (m, 1H), 2.37 (br d, J=1.3 Hz, 3H), 2.16-1.98 (m, 3H), 1.89-1.74 (m, 3H). LCMS Rt=2.769 min, m/z=628.2 [M+H]⁺.

LCMS (5 to 95% acetonitrile in water+0.03% ammonium bicarbonate over 6 mins) retention time 2.769 min, ESI+ found [M+H]=628.2.

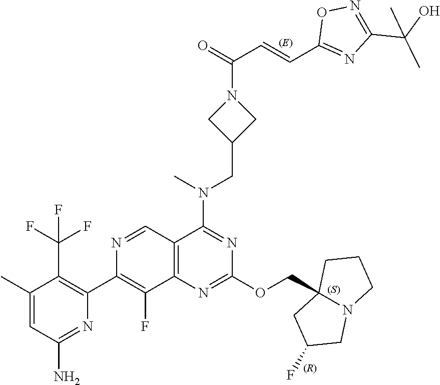

### Example 14: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

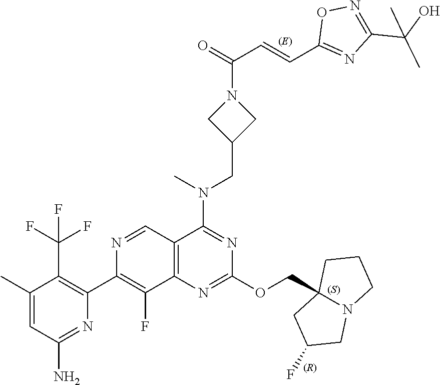

### Step 1: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The reaction mixture was purified by reverse phase HPLC (column: Waters Xbridge Prep OBD C18 150*40 mm*10 um; mobile phase: [water (NH4HCO3)-acetonitrile]; B %: 25%-50%, 8 min) affording (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one (25.02 mg, 18.44%) as a yellow solid: ¹H NMR (400 MHz, Acetonitrile-d3) δ 9.21 (s, 1H), 7.39-7.29 (m, 1H), 7.22-7.14 (m, 1H), 6.59 (s, 1H), 5.48 (s, 2H), 5.37-5.19 (m, 1H), 4.48 (t, J=8.6 Hz, 1H), 4.27 (br dd, J=5.4, 14.2 Hz, 2H), 4.22-4.13 (m, 4H), 3.95 (dd, J=5.6, 10.6 Hz, 1H), 3.57 (s, 3H), 3.18 (br d, J=13.0 Hz, 2H), 3.14-3.03 (m, 2H), 2.95-2.88 (m, 1H), 2.46 (br d, J=1.6 Hz, 3H), 2.13-2.05 (m, 3H), 1.93-1.85 (m, 3H), 1.59 (s, 6H). LCMS Rt=2.526 min, m/z=758.3 [M+H]⁺.

LCMS (5 to 95% acetonitrile in water+0.03% ammonium bicarbonate over 6 mins) retention time 2.526 min, ESI+ found [M+H]=758.3.

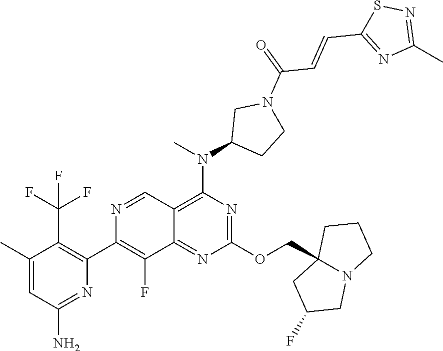

### Example 15: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-thiadiazol-5-yl)prop-2-en-1-one

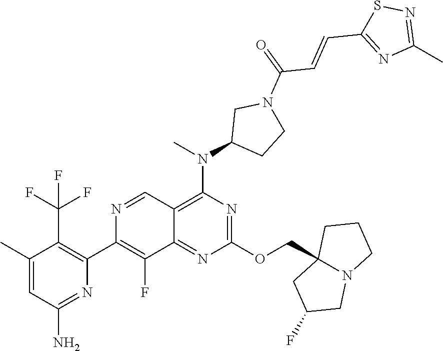

### Step 1: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-thiadiazol-5-yl)prop-2-en-1-one

The Horner-Wadsworth-Emmons reaction was prepared in a similar fashion to Example #2, Step 6. The residue was purified by reverse phase HPLC (column: Waters Xbridge BEH C18 100*30 mm*10 um; mobile phase: [water (NH4HCO3)-acetonitrile]; B %: 20%-50%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-thiadiazol-5-yl)prop-2-en-1-one (18.38 mg, 37.03%) as a white solid: ¹H NMR (400 MHz, Acetonitrile-d3) δ 9.14 (s, 1H), 7.80-7.68 (m, 1H), 7.39-7.24 (m, 1H), 6.57 (s, 1H), 5.53 (br d, J=6.4 Hz, 2H), 5.39-5.17 (m, 2H), 4.26-4.21 (m, 1H), 4.19-4.13 (m, 1H), 4.04-3.97 (m, 1H), 3.88-3.74 (m, 1H), 3.65-3.46 (m, 1H), 3.41 (s, 3H), 3.17-3.13 (m, 1H), 3.08 (br d, J=6.3 Hz, 1H), 2.95-2.85 (m, 1H), 2.66 (s, 1H), 2.63 (s, 1H), 2.45 (br d, J=1.6 Hz, 3H), 2.34-2.29 (m, 1H), 2.26-2.16 (m, 4H), 2.15-2.10 (m, 1H), 2.10-2.02 (m, 1H), 1.95-1.77 (m, 4H). LCMS Rt=2.810 min, m/z=730.3 [M+H]⁺.

LCMS (5 to 95% acetonitrile in water+0.1% ammonium bicarbonate over 6 mins) retention time 2.810 min, ESI+ found [M+H]=730.3.

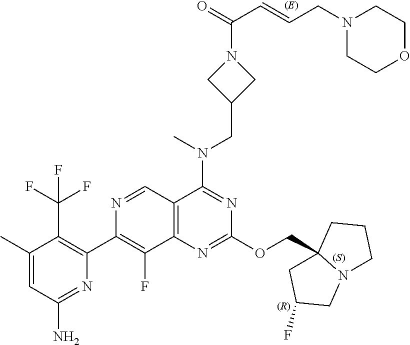

### Example 16: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-en-1-one

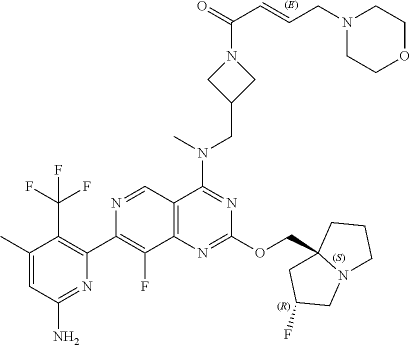

### Step 1: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-en-1-one

The Horner-Wadsworth-Emmons reaction was prepared in a similar fashion to Example #2, Step 6. The reaction mixture was purified by reverse phase HPLC (column: Waters Xbridge Prep OBD C18 150*40 mm*10 um; mobile phase: [water (NH4HCO3)-acetonitrile]; B %: 20%-50%, 8 min) affording (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-en-1-one (5.1 mg, 6.03%) as a yellow amorphous solid: ¹H NMR (400 MHz, Acetonitrile-d3) δ 9.20-9.12 (m, 1H), 6.68-6.53 (m, 2H), 6.09 (br d, J=15.0 Hz, 1H), 5.45 (br s, 2H), 5.35-5.16 (m, 1H), 4.29 (s, 1H), 4.18-4.05 (m, 6H), 3.83-3.78 (m, 1H), 3.63-3.58 (m, 4H), 3.53 (s, 3H), 3.18-3.12 (m, 3H), 3.07 (br d, J=4.9 Hz, 3H), 2.92-2.87 (m, 1H), 2.43 (br s, 4H), 2.37 (br s, 3H), 2.04 (br s, 3H), 1.89-1.73 (m, 3H). LCMS Rt=2.433 min, m/z=731.3 [M+H]⁺.

LCMS (5 to 95% acetonitrile in water+0.03% ammonium bicarbonate over 6 mins) retention time 2.433 min, ESI+ found [M+H]=731.3.

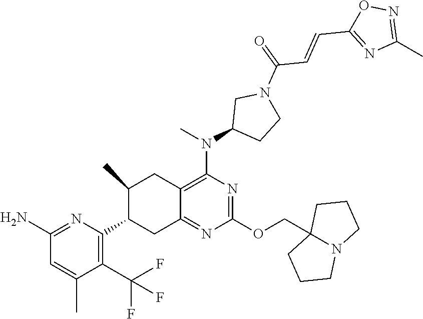 [SMILES: Cc1noc(/C=C/C(=O)N2CC[C@@H](N(C)c3nc(OCC45CCCN4CCC5)nc4c3C[C@H](C)[C@@H](c3nc(N)cc(C)c3C(F)(F)F)C4)C2)n1]

### Example 17: (E)-1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

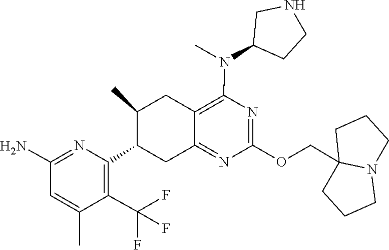 [SMILES: Cc1cc(N)nc([C@H]2Cc3nc(OCC45CCCN4CCC5)nc(N(C)[C@@H]4CCNC4)c3C[C@@H]2C)c1C(F)(F)F]

### Step 1: (6S,7S)-7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine

The deprotection of Boc and PMB was prepared in a similar fashion to Example #65, Step 15. The reaction mixture was concentrated in vacuo and purified by reverse phase HPLC (column: Phenomenex Luna 80*30 mm*3 um; mobile phase: [water (trifluroacetic acid)-acetoniitrile]; B %: 1%-30%, 8 min) affording (6S,7S)-7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine (110 mg, 97.98%, trifluoroacetate salt) as a white solid: LCMS Rt=0.571 min, m/z=559.3 [M+H]⁺.

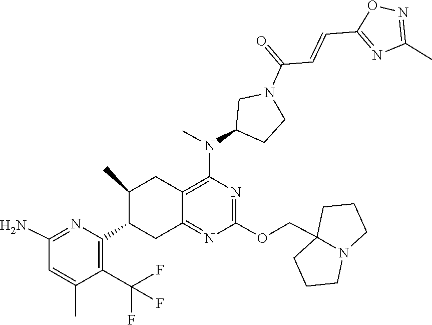 [SMILES: Cc1noc(/C=C/C(=O)N2CC[C@@H](N(C)c3nc(OCC45CCCN4CCC5)nc4c3C[C@H](C)[C@@H](c3nc(N)cc(C)c3C(F)(F)F)C4)C2)n1]

### Step 2: (E)-1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #65, Step 16. The crude product was purified by reverse phase HPLC (column: Waters Xbridge Prep OBD C18 150*40 mm*10 um; mobile phase: [water (NH4HCO3)-acetonitrile]; B %: 30%-70%, 8 min) affording (E)-1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (15.6 mg, 29.93%) as a yellow solid: 1H NMR (400 MHz, Acetonitrile-d3) δ 7.51-7.43 (m, 1H), 7.41-7.33 (m, 1H), 6.28 (s, 1H), 5.24 (s, 2H), 4.79-4.58 (m, 1H), 4.16 (dd, J=8.0, 10.3 Hz, 1H), 4.03-3.89 (m, 3H), 3.87-3.74 (m, 1H), 3.68 (br d, J=8.6 Hz, 1H), 3.52-3.36 (m, 1H), 3.17 (td, J=5.3, 10.3 Hz, 1H), 3.07-2.94 (m, 3H), 2.93 (d, J=3.1 Hz, 3H), 2.89-2.81 (m, 1H), 2.66 (s, 4H), 2.41 (s, 1H), 2.40 (s, 1H), 2.38 (d, J=3.5 Hz, 2H), 2.30-2.23 (m, 1H), 2.21-2.14 (m, 1H), 2.13-2.03 (m, 2H), 1.91 (td, J=6.1, 12.1 Hz, 2H), 1.86-1.71 (m, 4H), 1.66-1.54 (m, 2H), 0.77 (d, J=6.3 Hz, 3H). LCMS Rt=2.742 min, m/z=695.4 [M+H]⁺.

LCMS (5 to 95% acetonitrile in water+0.03% ammonium bicarbonate over 6 mins) retention time 2.742 min, ESI+ found [M+H]=695.4.

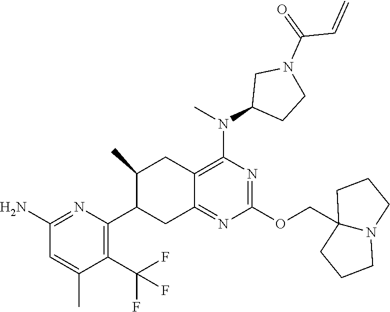 [SMILES: C=CC(=O)N1CC[C@@H](N(C)c2nc(OCC34CCCN3CCC4)nc3c2C[C@H](C)C(c2nc(N)cc(C)c2C(F)(F)F)C3)C1]

### Example 18: 1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

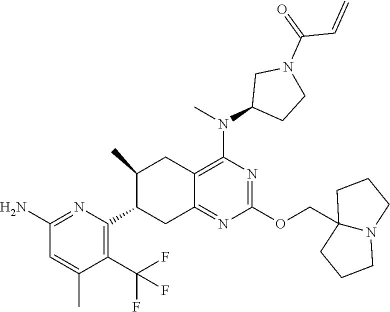 [SMILES: C=CC(=O)N1CC[C@@H](N(C)c2nc(OCC34CCCN3CCC4)nc3c2C[C@H](C)[C@@H](c2nc(N)cc(C)c2C(F)(F)F)C3)C1]

### Step 1: 1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

The amide coupling reaction was prepared in a similar fashion to Example #65, Step 16. The reaction mixture was concentrated in vacuo and purified by reverse phase HPLC (column: Waters Xbridge BEH C18 100*30 mm*10 um; mobile phase: [water (NH4HCO3)-acetonitrile]; B %: 35%-70%, 8 min) affording 1-((R)-3-(((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one (13.43 mg, 28.73%) as a yellow oil: ¹H NMR (400 MHz, Acetonitrile-d3) δ 6.59 (dd, J=10.3, 16.8 Hz, 1H), 6.34-6.16 (m, 2H), 5.67 (ddd, J=2.4, 5.0, 10.4 Hz, 1H), 5.20 (br s, 2H), 4.79-4.49 (m, 1H), 4.10-3.89 (m, 3H), 3.84-3.70 (m, 1H), 3.63-3.50 (m, 1H), 3.44-3.30 (m, 1H), 3.17 (td, J=5.2, 10.6 Hz, 1H), 2.96 (br dd, J=4.9, 9.7 Hz, 3H), 2.92 (d, J=4.6 Hz, 3H), 2.88-2.80 (m, 1H), 2.69-2.56 (m, 3H), 2.54-2.45 (m, 1H), 2.38 (q, J=3.5 Hz, 3H), 2.15-2.02 (m, 3H), 1.94-1.87 (m, 2H), 1.86-1.70 (m, 4H), 1.65-1.54 (m, 2H), 0.77 (d, J=6.4 Hz, 3H). LCMS Rt=2.818 min, m/z=613.3 [M+H]⁺.

LCMS (5 to 95% acetonitrile in water+0.03% ammonium bicarbonate over 6 mins) retention time 2.818 min, ESI+ found [M+H]=613.3.

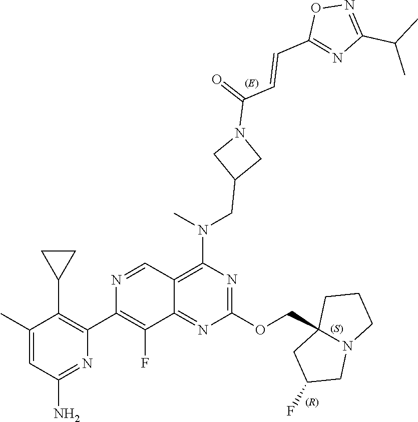

### Example 19: (E)-1-(3-(((7-(6-amino-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

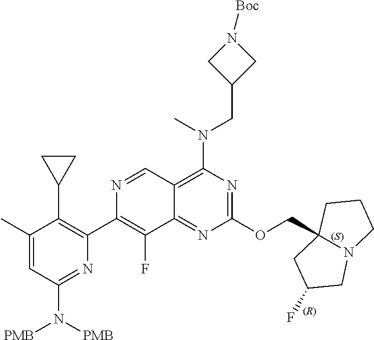

### Step 1: tert-butyl 3-(((7-(6-(bis(4-methoxybenzyl)amino)-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The residue was purified by column chromatography (silica gel, 100-200 mesh, 0-100% ethyl acetate in petroleum ether) affording tert-butyl 3-(((7-(6-(bis(4-methoxybenzyl)amino)-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (270 mg, crude) as a yellow gum, used in next step without any further purification. LCMS Rt=0.962 min, m/z=890.5 [M+H]⁺.

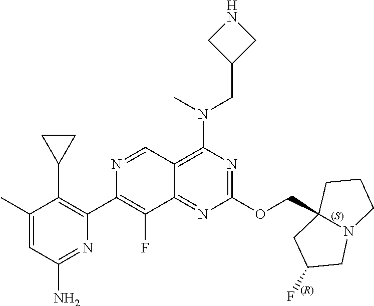

### Step 2: 7-(6-amino-3-cyclopropyl-4-methylpyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The mixture was purified by reverse phase HPLC (column: Phenomenex Luna C18 150*30 mm*5 um; mobile phase: [water (trifluroacetic acid)-acetoniitrile]; B %: 1%-30%, 8 min) affording 7-(6-amino-3-cyclopropyl-4-methylpyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (55 mg, 29.49%, trifluoroacetate salt) as a yellow oil. LCMS Rt=1.147 min, m/z=550.3 [M+H]⁺.

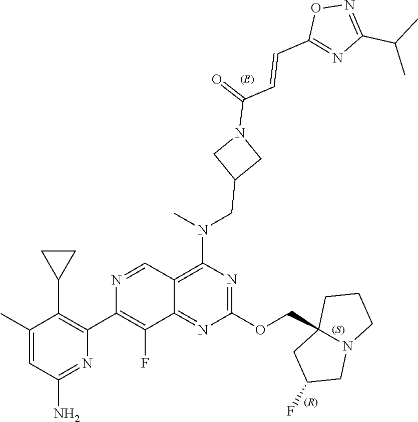

### Step 3: (E)-1-(3-(((7-(6-amino-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 20%-50%, 8 min) affording (E)-1-(3-(((7-(6-amino-3-cyclopropyl-4-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one (11.67 mg, 19.73%) as a white solid: ¹H NMR (400 MHz, Acetonitrile-d3) δ 9.24 (s, 1H), 7.37-7.30 (m, 1H), 7.21-7.13 (m, 1H), 6.52 (s, 1H), 5.40-5.15 (m, 1H), 4.54-4.47 (m, 1H), 4.34-4.10 (m, 6H), 4.01-3.93 (m, 1H), 3.62-3.57 (m, 3H), 3.33-3.17 (m, 1H), 3.17-3.06 (m, 4H), 2.93-2.86 (m, 1H), 2.49-2.36 (m, 3H), 2.26-2.01 (m, 3H), 1.93-1.69 (m, 4H), 1.37-1.22 (m, 6H), 0.55 (br d, J=8.5 Hz, 2H), −0.01 (br d, J=4.8 Hz, 2H). LCMS Rt=1.948 min, m/z=714.4 [M+H]⁺.

LCMS (5 to 95% acetonitrile in water+0.03% ammonium bicarbonate over 6 mins) retention time 1.948 min, ESI+ found [M+H]=714.4.

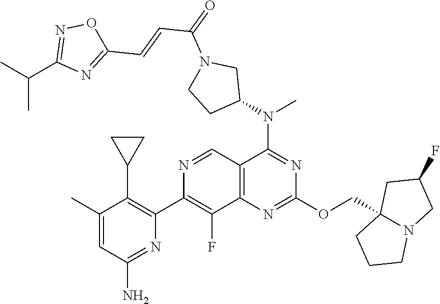 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1]

### Example 20: (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

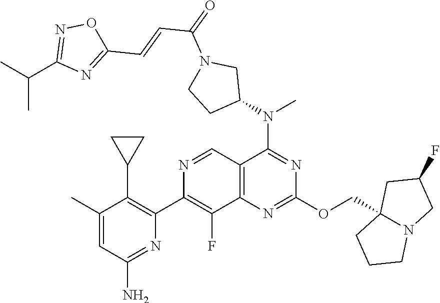 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1]

### Step 1: (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 25%-65%, 8 min) affording (E)-1-((R)-3-((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (13.64 mg, 27.82%) as a yellow solid. ¹H NMR (400 MHz, Acetonitrile-d3) δ 9.21 (d, J=2.1 Hz, 1H), 7.53-7.37 (m, 2H), 6.52-6.47 (m, 1H), 5.50-5.18 (m, 2H), 4.88-4.76 (m, 2H), 4.30-4.19 (m, 2H), 4.18-4.13 (m, 1H), 4.08-3.87 (m, 2H), 3.83-3.75 (m, 1H), 3.67-3.51 (m, 1H), 3.45 (s, 3H), 3.19-3.14 (m, 2H), 3.13-3.07 (m, 1H), 2.97-2.87 (m, 1H), 2.43 (s, 3H), 2.38-2.32 (m, 1H), 2.26-2.20 (m, 2H), 2.15-2.12 (m, 1H), 2.10-2.05 (m, 1H), 1.94-1.83 (m, 3H), 1.78-1.69 (m, 1H), 1.37 (d, J=7.0 Hz, 3H), 1.35 (d, J=7.0 Hz, 3H), 0.59-0.52 (m, 2H), 0.06-0.00 (m, 2H). LCMS Rt=2.918 min, m/z=714.4 [M+H]⁺.

LCMS (5 to 95% acetonitrile in water+0.03% ammonium bicarbonate over 6 min) retention time 2.918 min, ESI+ found [M+H]=714.4.

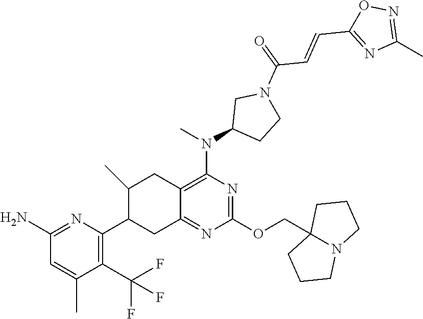 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1]

### Example 21: (E)-1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

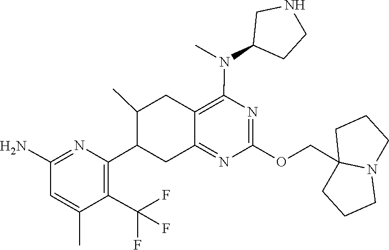 [SMILES: Cc1cc(N)nc(C2Cc3nc(OCC45CCCN4CCC5)nc(N(C)[C@@H]4CCNC4)c3CC2C)c1C(F)(F)F]

### Step 1: 7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine

The deprotection of Boc and PMB was prepared in a similar fashion to Example #65, Step 15. The reaction mixture was concentrated in vacuo and purified by reverse phase HPLC (column: Phenomenex Luna 80*30 mm*3 um; mobile phase: [water (trifluroacetic acid)-acetoniitrile]; B %: 1%-30%, 8 min) affording 7-(6-amino-4-methyl-3-(trifluoromethyl)pyridin-2-yl)-2-((hexahydro-1H-pyrrolizin-7a-yl)methoxy)-N,6-dimethyl-N—((R)-pyrrolidin-3-yl)-5,6,7,8-tetrahydroquinazolin-4-amine (70 mg, 93.52%, trifluoroacetate salt) as a white solid: LCMS Rt=0.453 min, m/z=559.3 [M+H]⁺.

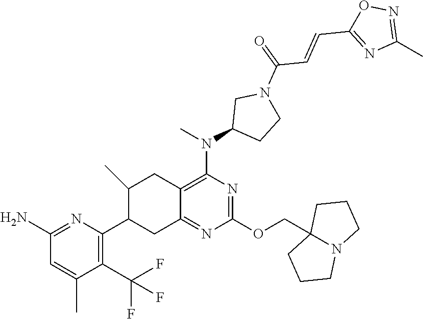 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1]

### Step 2: (E)-1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #65, Step 16. The crude product was purified by reverse phase HPLC (column: Waters Xbridge BEH C18 100*30 mm*10 um; mobile phase: [water (NH4HCO3)-acetonitrile]; B %: 35%-65%, 8 min) affording (E)-1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (8.23 mg, 19.92%) as a yellow solid: 1H NMR (400 MHz, Acetonitrile-d3) δ 7.50-7.43 (m, 1H), 7.40-7.30 (m, 1H), 6.27 (s, 1H), 5.32 (br s, 2H), 4.80-4.55 (m, 1H), 4.23-4.09 (m, 1H), 4.07-3.91 (m, 3H), 3.89-3.76 (m, 1H), 3.76-3.58 (m, 1H), 3.52-3.39 (m, 1H), 3.22-3.13 (m, 1H), 3.08-2.96 (m, 3H), 2.94-2.90 (m, 2H), 2.89-2.80 (m, 1H), 2.69-2.56 (m, 4H), 2.46 (br dd, J=4.9, 16.4 Hz, 3H), 2.41 (s, 1H), 2.37-2.33 (m, 2H), 2.20-2.03 (m, 3H), 1.95-1.87 (m, 2H), 1.86-1.71 (m, 4H), 1.67-1.52 (m, 2H), 0.75 (br d, J=6.4 Hz, 3H). LCMS Rt=2.748 min, m/z=695.4 [M+H]+.

LCMS (5 to 95% acetonitrile in water+0.03% ammonium bicarbonate over 6 mins) retention time 2.748 min, ESI+ found [M+H]=695.4.

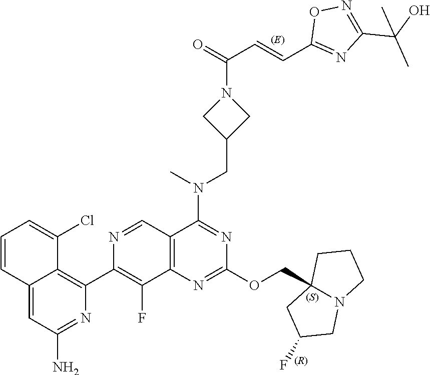

### Example 22: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

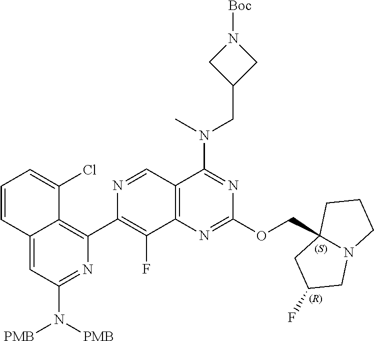

### Step 1: tert-butyl 3-(((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The mixture was purified by reverse phase HPLC(column: Phenomenex luna C18 250*50 mm*10 um; mobile phase: [water (trifluroacetic acid)-acetoniitrile]; B %: 45%-75%, 10 min) affording tert-butyl 3-(((7-(3-(bis(4-methoxybenzyl)amino)-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (400 mg, 25.85%, trifluoroacetic salt) as a yellow solid: ¹H NMR (400 MHz, Chloroform-d) δ 9.28 (s, 1H), 7.35 (d, J=8.4 Hz, 1H), 7.24-7.19 (m, 2H), 7.10 (br d, J=8.5 Hz, 4H), 6.76 (br d, J=8.5 Hz, 4H), 6.61 (s, 1H), 5.59-5.31 (m, 1H), 4.89-4.82 (m, 2H), 4.55-4.49 (m, 2H), 4.02 (br d, J=8.1 Hz, 2H), 3.75-3.68 (m, 6H), 3.57-3.38 (m, 4H), 3.28-3.17 (m, 1H), 3.04-2.94 (m, 1H), 2.79-2.63 (m, 2H), 2.54-2.37 (m, 3H), 2.30-2.15 (m, 3H), 1.57 (br d, J=7.4 Hz, 3H), 1.37 (s, 9H), 1.30 (br d, J=7.4 Hz, 3H). LCMS Rt=0.887 min, m/z=920.4 [M+H]⁺.

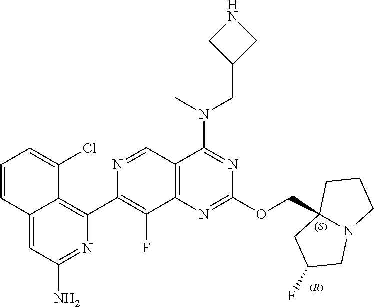

### Step 2: 7-(3-amino-8-chloroisoquinolin-1-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The mixture was concentrated in vacuo affording 7-(3-amino-8-chloroisoquinolin-1-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (100 mg, crude, trifluoroacetate salt) as a yellow oil. LCMS Rt=0.509 min, m/z=580.2 [M+H]⁺.

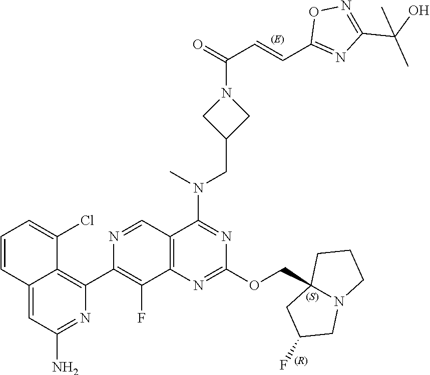

### Step 3: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 20%-50%, 8 min) affording (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one (11.93 mg, 13.62%) as a yellow amorphous solid: ¹H NMR (400 MHz, Acetonitrile-d3) δ 9.26-9.20 (m, 1H), 7.63 (d, J=8.4 Hz, 1H), 7.41 (t, J=7.8 Hz, 1H), 7.35-7.29 (m, 1H), 7.23 (br d, J=7.4 Hz, 1H), 7.19-7.14 (m, 1H), 6.92 (s, 1H), 5.33-5.13 (m, 3H), 4.47 (t, J=8.6 Hz, 1H), 4.31-4.06 (m, 7H), 3.93 (br dd, J=5.6, 10.3 Hz, 1H), 3.57 (s, 3H), 3.27-3.20 (m, 1H), 3.17-3.10 (m, 2H), 3.08-3.04 (m, 1H), 2.89 (br s, 1H), 2.06 (br d, J=17.8 Hz, 3H), 1.86 (br s, 3H), 1.57 (s, 6H). LCMS Rt=2.666 min, m/z=760.3 [M+H]⁺.

LCMS (5 to 95% acetonitrile in water+0.03% ammonium bicarbonate over 6 mins) retention time 2.666 min, ESI+ found [M+H]=760.3.

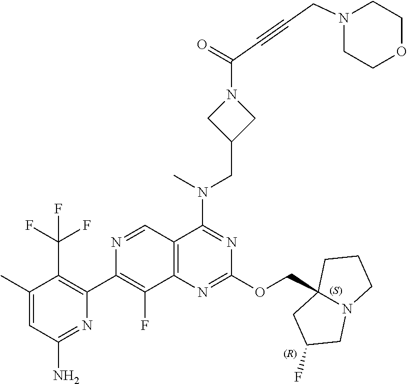

### Example 23: 1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-yn-1-one

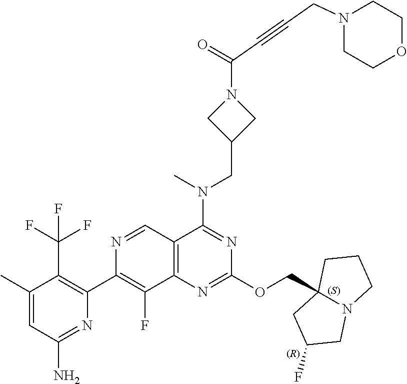

### Step 1: 1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-yn-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 20%-50%, 8 min) affording 1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-morpholinobut-2-yn-1-one (18.39 mg, 23.27%) as a yellow amorphous solid: ¹H NMR (400 MHz, Acetonitrile-d3) δ 9.19-9.14 (m, 1H), 6.56 (s, 1H), 5.44 (s, 2H), 5.35-5.18 (m, 1H), 4.28 (t, J=8.7 Hz, 1H), 4.22-4.00 (m, 6H), 3.82 (dd, J=5.6, 9.9 Hz, 1H), 3.64-3.58 (m, 4H), 3.53 (s, 3H), 3.43 (s, 2H), 3.23-3.11 (m, 3H), 3.06 (s, 1H), 2.93-2.85 (m, 1H), 2.49-2.45 (m, 4H), 2.43 (br d, J=1.4 Hz, 3H), 2.20-2.15 (m, 1H), 2.11 (br s, 2H), 1.91-1.79 (m, 3H). LCMS Rt=2.612 min, m/z=729.3 [M+H]⁺.

LCMS (5 to 95% acetonitrile in water+0.03% ammonium bicarbonate over 6 mins) retention time 2.612 min, ESI+ found [M+H]=729.3.

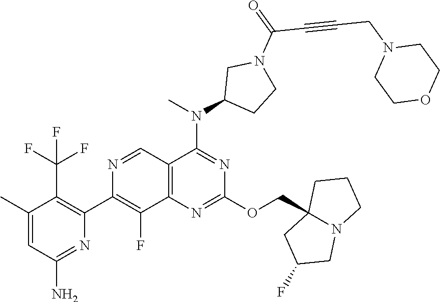 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F]

### Example 24: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-yn-1-one

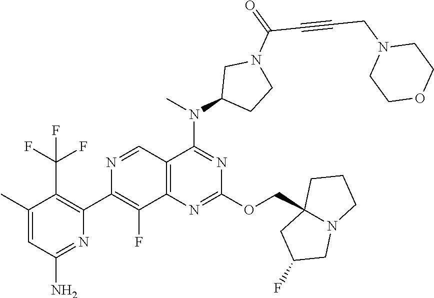 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F]

### Step 1: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-yn-1-one

The amide coupling reaction was prepared in a similar fashion to Example #71, Step 11. The residue was purified by reverse phase HPLC (column: 3_Phenomenex Luna C18 75*30 mm*3 um; mobile phase: [water (formic acid)-acetonitrile]; B %: 1%-30%, 8 min) affording 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-yn-1-one (1.3 mg, 1.75%, formic acid salt) as a yellow oil: ¹H NMR (400 MHz, Acetonitrile-d3) δ 9.20 (d, J=3.4 Hz, 1H), 6.60 (s, 1H), 5.54-5.46 (m, 2H), 5.44-5.38 (m, 1H), 4.63-4.52 (m, 1H), 4.19-3.96 (m, 1H), 3.94-3.79 (m, 1H), 3.77-3.72 (m, 1H), 3.70-3.62 (m, 9H), 3.52 (s, 1H), 3.50 (s, 1H), 3.46 (s, 1H), 3.44 (s, 1H), 3.28-3.14 (m, 1H), 2.54 (td, J=4.6, 15.1 Hz, 4H), 2.47 (br d, J=1.4 Hz, 3H), 2.41-2.33 (m, 2H), 2.01-1.99 (m, 2H), 1.87-1.79 (m, 6H). LCMS Rt=1.865 min, m/z=729.3 [M+H]⁺.

LCMS (5 to 95% acetonitrile in water+0.03% ammonium bicarbonate over 6 min) retention time 1.865 min, ESI+ found [M+H]=729.3.

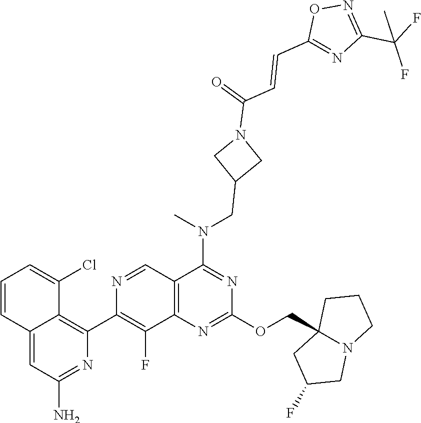 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12]

### Example 25: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

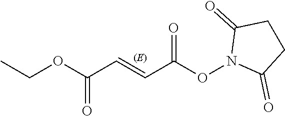 [SMILES: CCOC(=O)/C=C/C(=O)ON1C(=O)CCC1=O]

### Step 1: (E)-ethyl 4-(((E)-(1-amino-2,2-difluoropropylidene)amino)oxy)-4-oxobut-2-enoate

To a solution of (E)-4-ethoxy-4-oxo-but-2-enoic acid (5 g, 34.69 mmol), 1-hydroxypyrrolidine-2,5-dione (12 g, 104.27 mmol) in acetonitrile (100 mL) was added 1-(3-Dimethylaminopropyl)-3-ethylcarbodiimide (13.30 g, 69.38 mmol) at 0° C. The mixture was stirred at 25° C. for 12 h. The mixture was diluted with water (50 mL) and extracted with ethyl acetate (3×50 mL). The combined organic layers were dried over sodium sulphate and concentrated in vacuo affording (E)-ethyl 4-(((E)-(1-amino-2,2-difluoropropylidene)amino)oxy)-4-oxobut-2-enoate (16.8 g, crude) as a yellow oil, used into the next step without further purification.

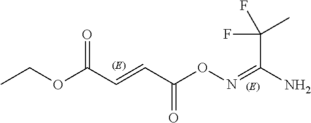 [SMILES: CCOC(=O)/C=C/C(=O)O/N=C(/N)C(C)(F)F]

### Step 2: (E)-ethyl 4-(((E)-(1-amino-2,2-difluoropropylidene)amino)oxy)-4-oxobut-2-enoate

The amide coupling reaction was prepared in a similar fashion to Example #71, Step 8. The combined organic layers were dried over sodium sulphate and concentrated in vacuo affording (E)-ethyl 4-(((E)-(1-amino-2,2-difluoropropylidene)amino)oxy)-4-oxobut-2-enoate (3.6 g, crude) as a brown oil, used in the next step without further purification. LCMS Rt=0.510 min, m/z=250.1 [M+H]⁺.

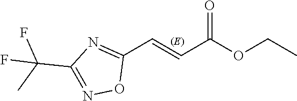 [SMILES: CCOC(=O)/C=C/c1nc(C(C)(F)F)no1]

### Step 3: ethyl (E)-3-[3-(1,1-difluoroethyl)-1,2,4-oxadiazol-5-yl]prop-2-enoate

The cyclization reaction was prepared in a similar fashion to Example #71, Step 9. The crude product was purified by column chromatography (silica gel, 100-200 mesh, 0-100% ethyl acetate in petroleum ether) affording ethyl (E)-3-[3-(1,1-difluoroethyl)-1,2,4-oxadiazol-5-yl]prop-2-enoate (1.1 g, 32.04%) as a white solid. LCMS Rt=0.633 min, m/z=232.1 [M+H]⁺.

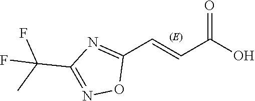

### Step 4: (E)-3-[3-(1,1-difluoroethyl)-1,2,4-oxadiazol-5-yl]prop-2-enoic acid

The hydrolysis reaction was prepared in a similar fashion to Example #71, Step 10. The reaction mixture were concentrated in vacuo affording (E)-3-[3-(1,1-difluoroethyl)-1,2,4-oxadiazol-5-yl]prop-2-enoic acid (1 g, crude) as a white solid, used in the next step without further purification. LCMS Rt=0.545 min, m/z=204.0 [M+H]⁺.

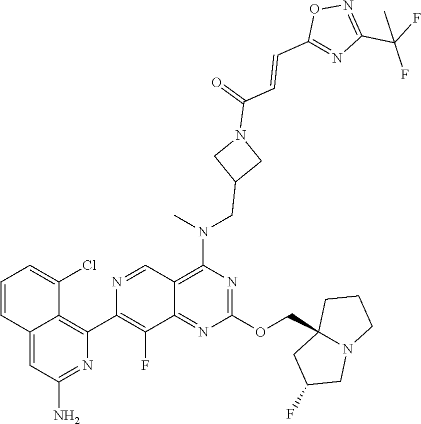 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12]

### Step 5: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 30%-60%, 8 min) affording (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one (11.06, 12.37%) as a yellow solid: ¹H NMR (400 MHz, Acetonitrile-d3) δ 9.27-9.20 (m, 1H), 7.63 (br d, J=8.4 Hz, 1H), 7.45-7.35 (m, 2H), 7.26 (br d, J=16.0 Hz, 2H), 6.92 (s, 1H), 5.33-5.12 (m, 3H), 4.48 (brt, J=8.8 Hz, 1H), 4.28-4.09 (m, 6H), 3.97-3.91 (m, 1H), 3.57 (s, 3H), 3.22 (br d, J=6.9 Hz, 1H), 3.16-3.10 (m, 2H), 3.06 (br s, 1H), 2.91-2.86 (m, 1H), 2.08 (br s, 3H), 2.03 (br s, 3H), 1.89-1.74 (m, 3H). LCMS Rt=2.701 min, m/z=766.3 [M+H]⁺.

LCMS (5 to 95% acetonitrile in water+0.03% ammonium bicarbonate over 6 mins) retention time 2.701 min, ESI+ found [M+H]=766.3.

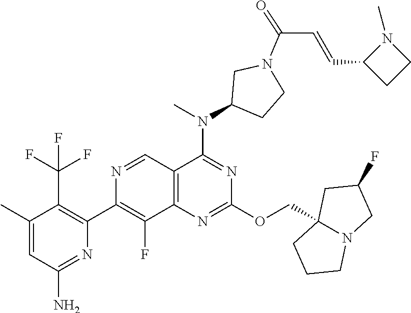 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F]

### Example 26: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((R)-1-methylazetidin-2-yl)prop-2-en-1-one

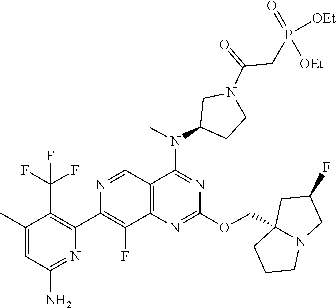 [SMILES: CCOP(=O)(CC(=O)N1CC[C@@H](N(C)c2nc(OC[C@@]34CCCN3C[C@H](F)C4)nc3c(F)c(-c4nc(N)cc(C)c4C(F)(F)F)ncc23)C1)OCC]

### Step 1: diethyl (2-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate

The amide coupling reaction was prepared in a similar fashion to Example #2, Step 5. The residue was purified by reverse phase HPLC(column: Waters Xbridge Prep OBD C18 150*40 mm*10 um; mobile phase: [water (NH4HCO3)-acetonitrile]; B %: 30%-60%, 8 min) affording diethyl (2-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-2-oxoethyl)phosphonate (7.77 mg, 14.68%) as a yellow oil. LCMS Rt=1.766 min, m/z=756.3 [M+H]⁺.

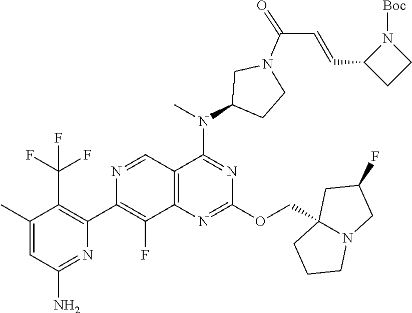 [SMILES: Cc1cc(N)nc(-c2ncc3c(N(C)[C@@H]4CCN(C(=O)/C=C/[C@H]5CCN5C(=O)OC(C)(C)C)C4)nc(OC[C@@]45CCCN4C[C@H](F)C5)nc3c2F)c1C(F)(F)F]

### Step 2: (R)-tert-butyl 2-((E)-3-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

The Horner-Wadsworth-Emmons reaction was prepared in a similar fashion to Example #2, Step 6. The reaction mixture was concentrated in vacuo affording (R)-tert-butyl 2-((E)-3-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (70 mg, crude) as a yellow oil, used in next step without any further purification. LCMS Rt=1.553 min, m/z=787.4 [M+H]⁺.

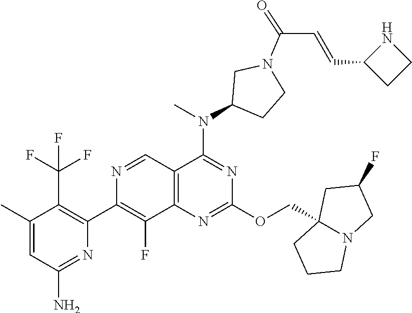 [SMILES: Cc1cc(N)nc(-c2ncc3c(N(C)[C@@H]4CCN(C(=O)/C=C/[C@H]5CCN5)C4)nc(OC[C@@]45CCCN4C[C@H](F)C5)nc3c2F)c1C(F)(F)F]

### Step 3: (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-azetidin-2-yl)prop-2-en-1-one

The deprotection of Boc group was prepared in a similar fashion to Example #71, Step 7. The reaction mixture was concentrated in affording (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((R)-azetidin-2-yl)prop-2-en-1-one (60 mg, crude, trifluoroacetate salt) as a yellow oil used in next step without any further purification. LCMS Rt=0.598 min, m/z=687.3 [M+H]⁺.

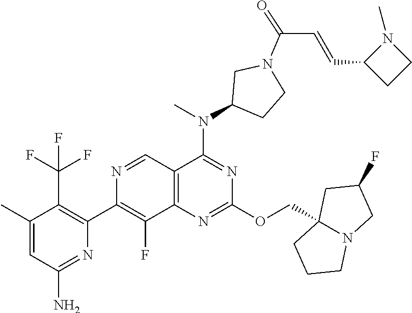 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F]

### Step 4: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((R)-1-methylazetidin-2-yl)prop-2-en-1-one

The reductive amination reaction was prepared in a similar fashion to Example #2, Step 8. The residue was purified by reverse phase HPLC(column: Waters Xbridge BEH C18 100*30 mm*10 um; mobile phase: [water (NH4HCO3)-acetonitrile]; B %: 25%-45%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((R)-1-methylazetidin-2-yl)prop-2-en-1-one (7.77 mg, 14.68%) as a yellow oil: ¹H NMR (400 MHz, Acetonitrile-d3) δ 9.14 (d, J=2.1 Hz, 1H), 6.78 (ddd, J=1.8, 5.7, 15.1 Hz, 1H), 6.58 (s, 1H), 6.39 (t, J=14.6 Hz, 1H), 5.50 (br s, 2H), 5.41-5.18 (m, 2H), 4.28-4.19 (m, 1H), 4.18-4.10 (m, 1H), 4.10-3.89 (m, 1H), 3.89-3.73 (m, 1H), 3.70-3.61 (m, 1H), 3.56 (br dd, J=4.8, 12.0 Hz, 1H), 3.40 (d, J=1.5 Hz, 3H), 3.34-3.27 (m, 1H), 3.16 (br d, J=7.6 Hz, 2H), 3.09 (s, 1H), 2.97-2.87 (m, 1H), 2.87-2.77 (m, 1H), 2.46 (d, J=1.5 Hz, 3H), 2.41-2.34 (m, 1H), 2.27 (s, 3H), 2.21-2.11 (m, 4H), 2.10-2.03 (m, 1H), 1.99 (br s, 1H), 1.95-1.80 (m, 4H). LCMS Rt=2.686 min, m/z=701.3 [M+H]⁺.

LCMS (5 to 95% acetonitrile in water+0.03% ammonium bicarbonate over 6 mins) retention time 2.686 min, ESI+ found [M+H]=701.3.

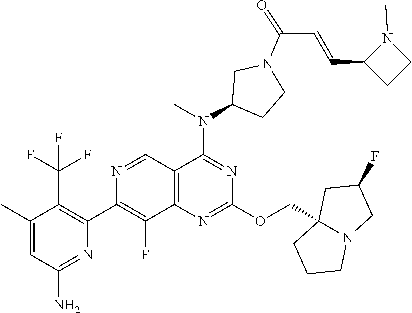 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F]

### Example 27: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((S)-1-methylazetidin-2-yl)prop-2-en-1-one

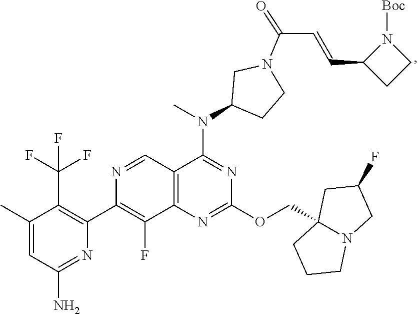 [SMILES: Cc1cc(N)nc(-c2ncc3c(N(C)[C@@H]4CCN(C(=O)/C=C/[C@@H]5CCN5C(=O)OC(C)(C)C)C4)nc(OC[C@@]45CCCN4C[C@H](F)C5)nc3c2F)c1C(F)(F)F]

### Step 1: (S)-tert-butyl 2-((E)-3-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate

The Horner-Wadsworth-Emmons reaction was prepared in a similar fashion to Example #2, Step 6. The reaction mixture was concentrated in vacuo affording (S)-tert-butyl 2-((E)-3-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-oxoprop-1-en-1-yl)azetidine-1-carboxylate (80 mg, crude) as a yellow oil used in next step without any further purification. LCMS Rt=0.572 min, m/z=787.4 [M+H]⁺.

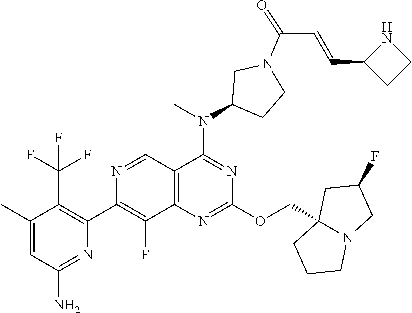 [SMILES: Cc1cc(N)nc(-c2ncc3c(N(C)[C@@H]4CCN(C(=O)/C=C/[C@@H]5CCN5)C4)nc(OC[C@@]45CCCN4C[C@H](F)C5)nc3c2F)c1C(F)(F)F]

### Step 4: (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one

The deprotection of Boc group was prepared in a similar fashion to Example 2, Step 7, the reaction mixture was concentrated in vacuo affording (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-((S)-azetidin-2-yl)prop-2-en-1-one (80 mg, crude, trifluoroacetate salt) as a yellow oil, used in next step without any further purification. LCMS Rt=0.454 min, m/z=687.3 [M+H]⁺.

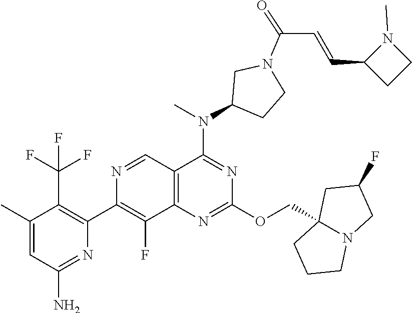 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F]

### Step 5: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((S)-1-methylazetidin-2-yl)prop-2-en-1-one

The reductive amination reaction was prepared in a similar fashion to Example #2, Step 8. The residue was purified by reverse phase HPLC(column: Waters Xbridge BEH C18 100*30 mm*10 um; mobile phase: [water (NH4HCO3)-acetonitrile]; B %: 25%-60%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-((S)-1-methylazetidin-2-yl)prop-2-en-1-one (19.85 mg, 27.79%) as a yellow oil: ¹H NMR (400 MHz, Acetonitrile-d3) δ 9.05-9.00 (m, 1H), 6.67 (td, J=5.2, 15.1 Hz, 1H), 6.58-6.45 (m, 1H), 6.33-6.19 (m, 1H), 5.44 (br d, J=6.6 Hz, 2H), 5.28-5.06 (m, 2H), 4.15-4.07 (m, 1H), 4.06-4.01 (m, 1H), 3.98-3.79 (m, 1H), 3.78-3.65 (m, 1H), 3.55-3.42 (m, 2H), 3.28 (br d, J=4.6 Hz, 3H), 3.24-3.16 (m, 1H), 3.05 (br d, J=5.8 Hz, 2H), 2.98 (br s, 1H), 2.83-2.77 (m, 1H), 2.76-2.67 (m, 1H), 2.34 (br s, 3H), 2.28-2.24 (m, 1H), 2.23-2.15 (m, 3H), 2.15-2.12 (m, 2H), 2.11-2.07 (m, 1H), 2.06-2.00 (m, 2H), 1.99-1.94 (m, 1H), 1.79 (br dd, J=10.3, 19.0 Hz, 4H). LCMS Rt=1.802 min, m/z=701.3 [M+H]⁺.

LCMS (5 to 95% acetonitrile in water+0.03% ammonium bicarbonate over 6 mins) retention time 1.802 min, ESI+ found [M+H]=701.3.

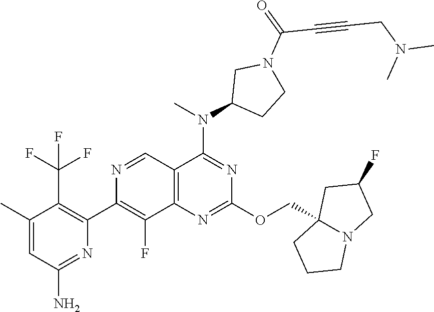

### Example 28: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-(dimethylamino)but-2-yn-1-one

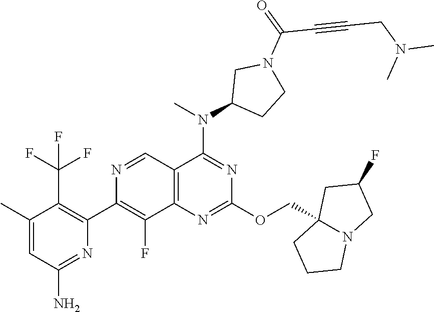

### Step 1: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-(dimethylamino)but-2-yn-1-one

The amide coupling reaction was prepared in a similar fashion to Example #71, Step 11. The residue was purified by reverse phase HPLC (column: Waters Xbridge BEH C18 100*30 mm*10 um; mobile phase: [water (NH×4HCO3)-acetonitrile]; B %: 30%-65%, 8 min) affording 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-(dimethylamino)but-2-yn-1-one (1.8 mg, 2.26%) as a yellow oil: ¹H NMR (400 MHz, Acetonitrile-d3) δ 9.15 (d, J=4.6 Hz, 1H), 8.13 (s, 1H), 6.59 (s, 1H), 5.51-5.42 (m, 2H), 5.40-5.20 (m, 2H), 4.26-4.21 (m, 1H), 4.20-4.15 (m, 1H), 4.15-3.96 (m, 1H), 3.90 (dd, J=8.4, 13.1 Hz, 1H), 3.85-3.75 (m, 1H), 3.74-3.61 (m, 1H), 3.52-3.47 (m, 1H), 3.47-3.44 (m, 1H), 3.23-3.14 (m, 2H), 3.10 (s, 1H), 2.97-2.89 (m, 1H), 2.46 (br d, J=1.3 Hz, 3H), 2.41-2.34 (m, 3H), 2.33-2.29 (m, 6H), 2.12-2.05 (m, 3H), 1.93-1.84 (m, 4H), 1.80 (td, J=2.5, 4.9 Hz, 1H). LCMS Rt=1.811 min, m/z=687.3 [M+H]⁺.

LCMS (5 to 95% acetonitrile in water+0.1% trifluoroacetic acid over 6 mins) retention time 1.811 min, ESI+ found [M+H]=687.3.

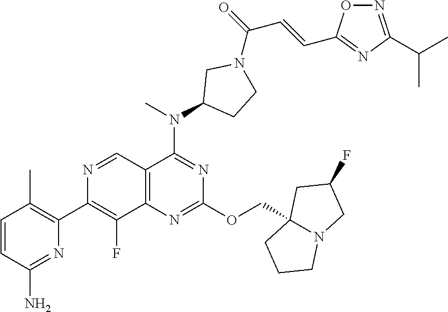 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F]

### Example 29: (E)-1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

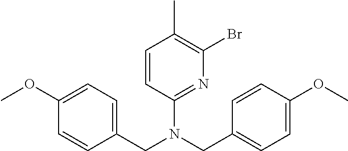

### Step 1: 6-bromo-N,N-bis[(4-methoxyphenyl)methyl]-5-methyl-pyridin-2-amine

The PMB protection reaction was prepared in a similar fashion to Example #71, Step 1. The crude product was purified by column chromatography (silica gel, 100-200 mesh, 60-75% petroleum ether in n-heptane) affording 6-bromo-N,N-bis[(4-methoxyphenyl)methyl]-5-methyl-pyridin-2-amine (3 g, 69.11%) as a yellow oil. LCMS Rt=0.967 min, m/z=426.1 [M+H]⁺.

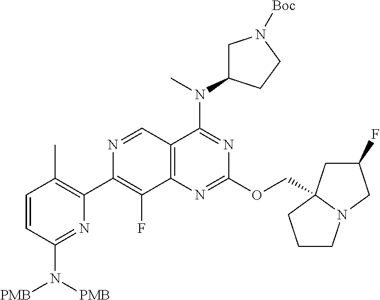

### Step 2: (R)-tert-butyl 3-((7-(6-(bis(4-methoxybenzyl)amino)-3-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by reverse phase HPLC (column: Phenomenex Luna 80*30 mm*3 um; mobile phase: [water (trifluroacetic acid)-acetoniitrile]; B %: 30%-60%, 8 min) affording (R)-tert-butyl 3-((7-(6-(bis(4-methoxybenzyl)amino)-3-methylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (200 mg, 32.90%, trifluoroacetate salt) as a white solid. LCMS Rt=0.799 min, m/z=850.4 [M+H]⁺.

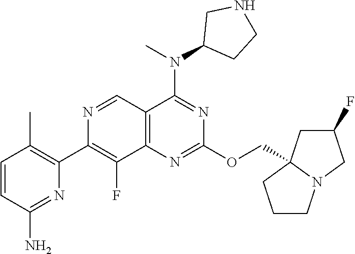 [SMILES: Cc1ccc(N)nc1-c1ncc2c(N(C)[C@@H]3CCNC3)nc(OC[C@@]34CCCN3C[C@H](F)C4)nc2c1F]

### Step 3: 7-(6-amino-3-methylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and PMB group was prepared in a similar fashion to Example #71, Step 7. The residue was purified by reverse phase HPLC (column: Phenomenex Luna 80*30 mm*3 um; mobile phase: [water (trifluroacetic acid)-acetonitrile]; B %: 1%-25%, 8 min) affording 7-(6-amino-3-methylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (100 mg, 68.12%, trifluoroacetate salt) as a yellow solid. LCMS Rt=0.357 min, m/z=510.3 [M+H]⁺.

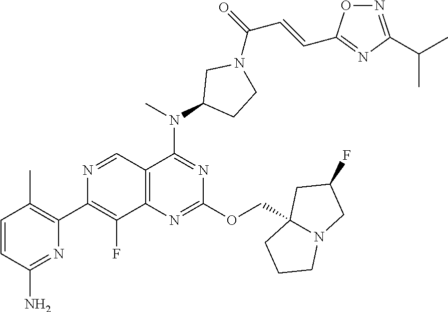 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F]

### Step 4: (E)-1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 20%-60%, 8 min) affording (E)-1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (6.88 mg, 6.31%) as a yellow oil: ¹H NMR (400 MHz, Acetonitrile-d3) δ 9.22 (s, 1H), 7.57-7.32 (m, 3H), 6.60 (d, J=8.4 Hz, 1H), 5.48-5.21 (m, 2H), 4.90-4.79 (m, 2H), 4.33-4.20 (m, 2H), 4.03 (br dd, J=8.4, 13.1 Hz, 1H), 3.95-3.84 (m, 1H), 3.82-3.74 (m, 1H), 3.66-3.51 (m, 1H), 3.44 (s, 3H), 3.32-3.08 (m, 4H), 3.03-2.92 (m, 1H), 2.48-2.30 (m, 4H), 2.24 (br d, J=2.8 Hz, 4H), 2.08 (s, 3H), 1.35 (dd, J=7.1, 7.8 Hz, 6H). LCMS Rt=2.766 min, m/z=674.3 [M+H]⁺.

LCMS (5 to 95% acetonitrile in water+0.03% ammonium bicarbonate over 6 mins) retention time 2.766 min, ESI+ found [M+H]=674.3.

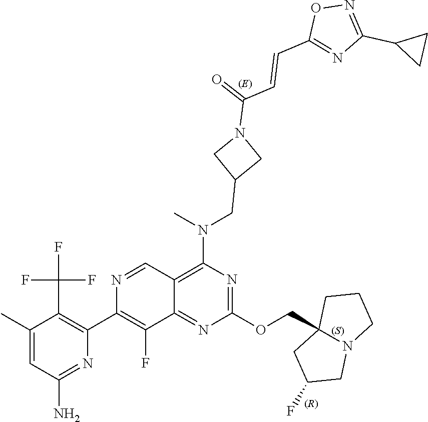

### Example 30: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-cyclopropyl-1,2,4-oxadiazol-5-yl)prop-2-en-1-one

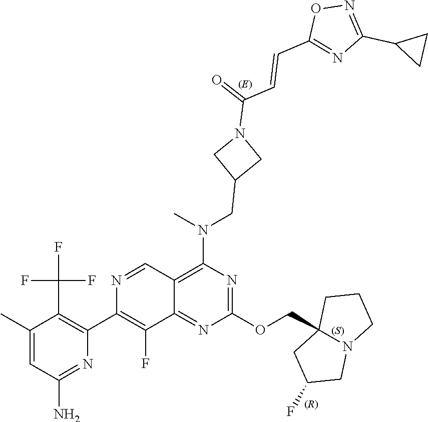

### Step 1: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-cyclopropyl-1,2,4-oxadiazol-5-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 35%-65%, 8 min) affording (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-cyclopropyl-1,2,4-oxadiazol-5-yl)prop-2-en-1-one (5.63 mg, 4.39%) as a white solid: ¹H NMR (400 MHz, Acetonitrile-d3) δ 9.21 (s, 1H), 7.18 (q, J=15.8 Hz, 2H), 6.81 (s, 2H), 6.51 (s, 1H), 5.35-5.15 (m, 1H), 4.48 (t, J=8.6 Hz, 1H), 4.30 (br dd, J=5.8, 8.8 Hz, 1H), 4.22-3.98 (m, 5H), 3.90 (br dd, J=5.7, 10.4 Hz, 1H), 3.56 (s, 3H), 3.23-3.12 (m, 1H), 3.10-2.97 (m, 3H), 2.86-2.76 (m, 1H), 2.37 (br d, J=1.1 Hz, 3H), 2.21-2.07 (m, 2H), 2.06-1.94 (m, 2H), 1.85-1.72 (m, 3H), 1.13-1.06 (m, 2H), 0.96-0.88 (m, 2H). LCMS Rt=2.669 min, m/z=740.3 [M+H]⁺.

LCMS (5 to 95% acetonitrile in water+0.03% ammonium bicarbonate over 6 mins) retention time 2.669 min, ESI+ found [M+H]=740.3.

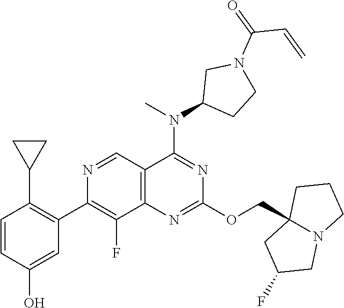 [SMILES: C=CC(=O)N1CC[C@@H](N(C)c2nc(OC[C@@]34CCCN3C[C@H](F)C4)nc3c(F)c(-c4cc(O)ccc4C4CC4)ncc23)C1]

### Example 31: 1-((R)-3-((7-(2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

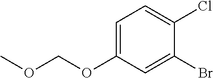

### Step 1: 2-bromo-1-iodo-4-(methoxymethoxy)benzene

To a solution of 3-bromo-4-iodo-phenol (2 g, 6.69 mmol) in acetone (20 mL) was added potassium carbonate (3.70 g, 26.76 mmol), then added chloro(methoxy)methane (1.08 g, 13.38 mmol) at 0° C. and the mixture was stirred 25° C. for 1 h under nitrogen atmosphere. The mixture was diluted with water (50 mL) and extracted with ethyl acetate (3×50 mL). The combined organic layers were dried over sodium sulphate and concentrated in vacuo affording 2-bromo-1-iodo-4-(methoxymethoxy)benzene (2.3 g, crude) as a yellow oil used in the next step without further purification.

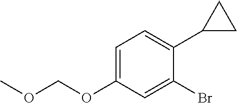

### Step 2: 2-bromo-1-cyclopropyl-4-(methoxymethoxy)benzene

To a solution of 2-bromo-1-iodo-4-(methoxymethoxy)benzene (800 mg, 2.33 mmol), cyclopropylboronic acid (400.74 mg, 4.67 mmol), cesium carbonate (2.28 g, 7.00 mmol) in dioxane (8 mL) and water (0.8 mL) was added cyclopenta-2,4-dien-1-yl(diphenyl)phosphane;dichloropalladium;iron(2+) (170.68 mg, 233.27 umol), the reaction was stirred at 100° C. for 2 h under nitrogen atmosphere. The mixture was diluted with water (20 mL), extracted with ethyl acetate (20 mL×3). The combined organic layers were concentrated to dryness in vacuo and purified by column chromatography (silica gel, 100-200 mesh, 0-20% ethyl acetate in petroleum ether) affording 2-bromo-1-cyclopropyl-4-(methoxymethoxy)benzene (440 mg, 73.36%) as a white oil.

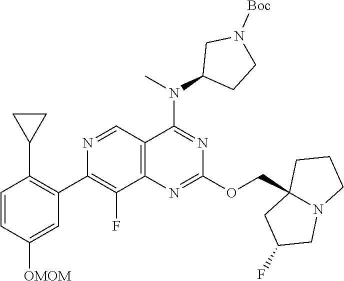

### Step 3: (R)-tert-butyl 3-((7-(2-cyclopropyl-5-(methoxymethoxy)phenyl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by reverse phase HPLC (column: Phenomenex luna C18 250*50 mm*10 um; mobile phase: [water (trifluroacetic acid)-acetoniitrile]; B %: 30%-70%, 10 min) affording (R)-tert-butyl 3-((7-(2-cyclopropyl-5-(methoxymethoxy)phenyl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (110 mg, 25.65%, trifluoroacetate salt) as a yellow oil. LCMS Rt=0.805 min, m/z=680.4 [M+H]⁺.

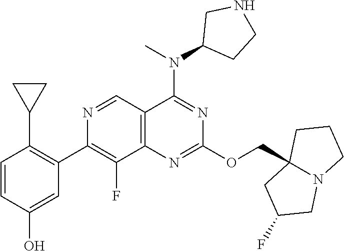 [SMILES: CN(c1nc(OC[C@@]23CCCN2C[C@H](F)C3)nc2c(F)c(-c3cc(O)ccc3C3CC3)ncc12)[C@@H]1CCNC1]

### Step 4: 4-cyclopropyl-3-(8-fluoro-2-(((2R,7aS)-2-fluorohexahydro-1H-pyrrolizin-7a-yl)methoxy)-4-(methyl((R)-pyrrolidin-3-yl)amino)pyrido[4,3-d]pyrimidin-7-yl)phenol

The deprotection of Boc and MOM group was prepared in a similar fashion to Example #71, Step 7. The reaction mixture was concentrated in vacuo affording 4-cyclopropyl-3-(8-fluoro-2-(((2R,7aS)-2-fluorohexahydro-1H-pyrrolizin-7a-yl)methoxy)-4-(methyl((R)-pyrrolidin-3-yl)amino)pyrido[4,3-d]pyrimidin-7-yl)phenol (84 mg, crude, hydrochloride salt) as a yellow oil used in next step without any further purification. LCMS Rt=0.587 min, m/z=536.3 [M+H]⁺.

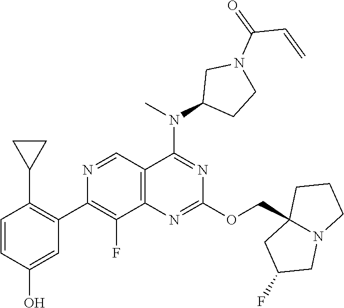 [SMILES: C=CC(=O)N1CC[C@@H](N(C)c2nc(OC[C@@]34CCCN3C[C@H](F)C4)nc3c(F)c(-c4cc(O)ccc4C4CC4)ncc23)C1]

### Step 5: 1-((R)-3-((7-(2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC(column: Phenomenex Luna C18 75*30 mm*3 um; mobile phase: [water (formic acid)-acetonitrile]; B %: 1%-50%, 8 min) affording 1-((R)-3-((7-(2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (2.22 mg, 2.38%, formic acid salt) as a yellow solid: ¹H NMR (400 MHz, Acetonitrile-d3) δ 9.19 (s, 1H), 8.13 (s, 1H), 6.94 (d, J=8.4 Hz, 1H), 6.86-6.77 (m, 2H), 6.65-6.50 (m, 1H), 6.23 (td, J=2.8, 16.9 Hz, 1H), 5.72-5.61 (m, 1H), 5.44-5.17 (m, 2H), 4.27-4.12 (m, 2H), 4.09-3.76 (m, 2H), 3.70-3.43 (m, 2H), 3.40 (s, 3H), 3.27-3.08 (m, 3H), 2.96-2.88 (m, 1H), 2.32-2.19 (m, 4H), 2.15-2.06 (m, 2H), 1.90-1.80 (m, 2H), 1.79-1.70 (m, 1H), 0.70-0.59 (m, 2H), 0.51-0.40 (m, 2H). LCMS Rt=1.992 min, m/z=590.3 [M+H]⁺.

LCMS (5 to 95% acetonitrile in water+0.1% trifluoroacetic acid over 6 mins) retention time 1.992 min, ESI+ found [M+H]=590.3.

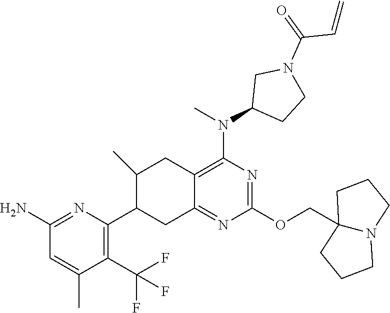 [SMILES: C=CC(=O)N1CC[C@@H](N(C)c2nc(OCC34CCCN3CCC4)nc3c2CC(C)C(c2nc(N)cc(C)c2C(F)(F)F)C3)C1]

### Example 32: 1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

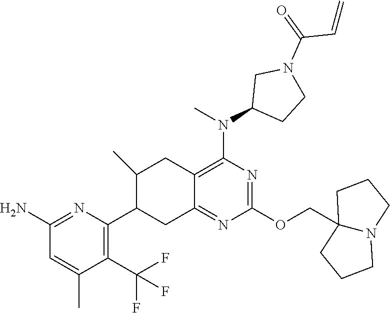 [SMILES: C=CC(=O)N1CC[C@@H](N(C)c2nc(OCC34CCCN3CCC4)nc3c2CC(C)C(c2nc(N)cc(C)c2C(F)(F)F)C3)C1]

### Step 1: 1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

The amide coupling reaction was prepared in a similar fashion to Example #65, Step 16. The reaction mixture was concentrated in vacuo and purified by reverse phase HPLC (column: Waters Xbridge BEH C18 100*30 mm*10 um; mobile phase: [water (NH4HCO3)-acetonitrile]; B %: 22%-62%, 8 min) affording 1-((3R)-3-((7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one (6.83 mg, 24.99%) as a yellow oil: ¹H NMR (400 MHz, Acetonitrile-d3) δ 66.66-6.45 (m, 1H), 6.33-6.14 (m, 2H), 5.78-5.54 (m, 1H), 5.23 (br s, 2H), 4.79-4.47 (m, 1H), 4.11-3.85 (m, 3H), 3.84-3.69 (m, 1H), 3.66-3.29 (m, 2H), 3.24-3.12 (m, 1H), 3.07-3.00 (m, 1H), 2.96 (br dd, J=5.1, 10.1 Hz, 2H), 2.93-2.89 (m, 3H), 2.88-2.80 (m, 1H), 2.72-2.55 (m, 3H), 2.54-2.43 (m, 1H), 2.40-2.34 (m, 3H), 2.19-2.02 (m, 3H), 1.95-1.87 (m, 2H), 1.87-1.71 (m, 4H), 1.65-1.52 (m, 2H), 0.77 (br d, J=6.4 Hz, 3H). LCMS Rt=2.603 min, m/z=613.3 [M+H]⁺.

LCMS (5 to 95% acetonitrile in water+0.03% ammonium bicarbonate over 6 mins) retention time 2.603 min, ESI+ found [M+H]=613.3.

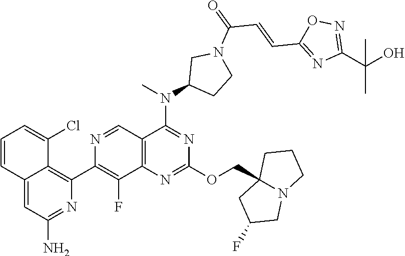 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1]

### Example 33: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

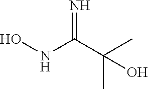

### Step 1: N,2-dihydroxy-2-methylpropanimidamide

To a solution of 2-hydroxy-2-methyl-propanenitrile (5 g, 58.75 mmol) in ethanol (50 mL) were added hydroxylamine hydrochloride (4.90 g, 70.50 mmol) and potassium carbonate (24.36 g, 176.25 mmol), the mixture was stirred at 80° C. for 2 h. The reaction mixture was concentrated in vacuo. The resulting residue was purified by column chromatography (silica gel, 100-200 mesh, 70-90% ethyl acetate in petroleum ether) affording N,2-dihydroxy-2-methylpropanimidamide (2.2 g, 31.7%) as a white solid. LCMS Rt=0.351 min, m/z=118.1 [M+H]⁺.

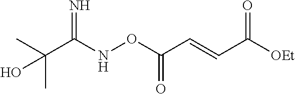 [SMILES: CCOC(=O)/C=C/C(=O)ONC(=N)C(C)(C)O]

### Step 2: (E)-ethyl 4-((2-hydroxy-2-methylpropanimidamido)oxy)-4-oxobut-2-enoate

The coupling reaction was prepared in a similar fashion to Example #71, Step 8. The crude product was purified by column chromatography (silica gel, 100-200 mesh, 70-90% ethyl acetate in petroleum ether) affording (E)-ethyl 4-((2-hydroxy-2-methylpropanimidamido)oxy)-4-oxobut-2-enoate (2.2 g, 49.89%) as a white solid. LCMS Rt=0.552 min, m/z=244.1 [M+H]⁺.

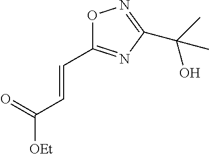 [SMILES: CCOC(=O)/C=C/c1nc(C(C)(C)O)no1]

### Step 3: (E)-ethyl 3-(3-(2-hydroxypropan-2-yl)-1,2,4-oxadiazol-5-yl)acrylate

The cyclization reaction was prepared in a similar fashion to Example #71, Step 9. The crude product was purified by column chromatography (silica gel, 100-200 mesh, 70-90% ethyl acetate in petroleum ether) affording (E)-ethyl 3-(3-(2-hydroxypropan-2-yl)-1,2,4-oxadiazol-5-yl)acrylate (370 mg, 26.63%) as a white solid: ¹H NMR (400 MHz, Chloroform-d) δ 7.41 (d, J=16.0 Hz, 1H), 6.97 (d, J=16.0 Hz, 1H), 4.31-4.17 (m, 2H), 1.64-1.55 (m, 6H), 1.34-1.23 (m, 3H). LCMS Rt=0.590 min, m/z=226.1 [M+H]⁺.

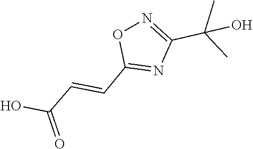

### Step 4: (E)-3-(3-(2-hydroxypropan-2-yl)-1,2,4-oxadiazol-5-yl)acrylic acid

The hydrolysis reaction was prepared in a similar fashion to Example #71, Step 10. The combined organic layers were dried over sodium sulphate and concentrated in vacuo affording (E)-3-(3-(2-hydroxypropan-2-yl)-1,2,4-oxadiazol-5-yl)acrylic acid (180 mg, 55.94%) as a white solid used in the next step without further purification. LCMS Rt=0.467 min, m/z=198.1 [M+H]⁺.

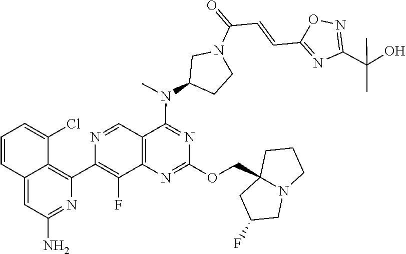 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1]

### Step 5: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The crude was purified by reverse phase reverse phase HPLC (column: Waters Xbridge Prep OBD C18 150*40 mm*10 um; mobile phase: [water (NH4HCO3)-acetonitrile]; B %: 30%-60%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one (15.43 mg, 21.95%) as a yellow amorphous solid: ¹H NMR (400 MHz, Acetonitrile-d3) δ 9.22-9.13 (m, 1H), 7.67 (br d, J=4.0 Hz, 1H), 7.52-7.32 (m, 3H), 7.26-7.20 (m, 1H), 6.93-6.89 (m, 1H), 5.46-5.33 (m, 1H), 5.32 (br s, 1H), 5.17 (br s, 2H), 4.25-4.10 (m, 3H), 4.05-3.95 (m, 1H), 3.80-3.70 (m, 1H), 3.46 (br s, 2H), 3.44-3.38 (m, 3H), 3.21-3.07 (m, 2H), 2.94-2.81 (m, 1H), 2.46-2.37 (m, 1H), 2.36-2.28 (m, 1H), 2.12-2.00 (m, 3H), 1.91-1.78 (m, 3H), 1.57 (d, J=9.1 Hz, 6H). LCMS Rt=2.697 min, m/z=760.3 [M+H]⁺.

LCMS (5 to 95% acetonitrile in water+0.1% ammonium bicarbonate over 6 mins) retention time 2.697 min, ESI+ found [M+H]=760.3.

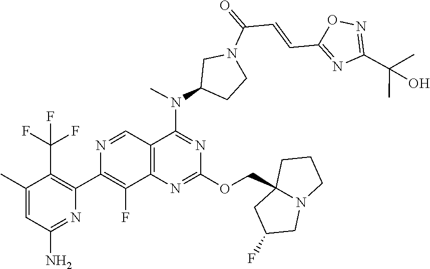 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F]

### Example 34: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

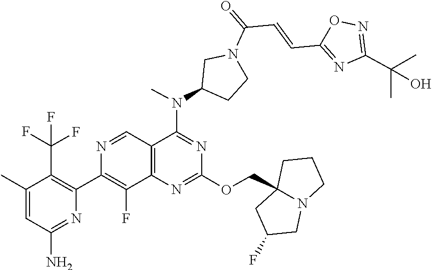 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F]

### Step 1: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The residue was purified by reverse phase HPLC (column: Phenomenex C18 75*30 mm*3 um; mobile phase: [water (NH4HCO3)-acetonitrile]; B %: 5%-55%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one (13.38 mg, 20.41%) as a yellow solid: ¹H NMR (400 MHz, Acetonitrile-d3) δ 9.16 (d, J=1.6 Hz, 1H), 7.55-7.36 (m, 2H), 6.59 (s, 1H), 5.48 (br s, 2H), 5.35-5.19 (m, 1H), 4.28-4.12 (m, 3H), 4.02 (br dd, J=8.0, 12.9 Hz, 1H), 3.95-3.84 (m, 1H), 3.82-3.72 (m, 1H), 3.66-3.48 (m, 2H), 3.42 (s, 3H), 3.23-3.13 (m, 2H), 3.09 (br d, J=1.6 Hz, 1H), 2.97-2.86 (m, 1H), 2.46 (br d, J=1.6 Hz, 3H), 2.42-2.29 (m, 2H), 2.16-2.11 (m, 2H), 2.09-2.05 (m, 1H), 1.93-1.84 (m, 3H), 1.60 (d, J=8.5 Hz, 6H). LCMS Rt=2.536 min, m/z=758.3 [M+H]⁺.

LCMS (5 to 95% acetonitrile in water+0.1% ammonium bicarbonate over 6 mins) retention time 2.536 min, ESI+ found [M+H]=758.3.

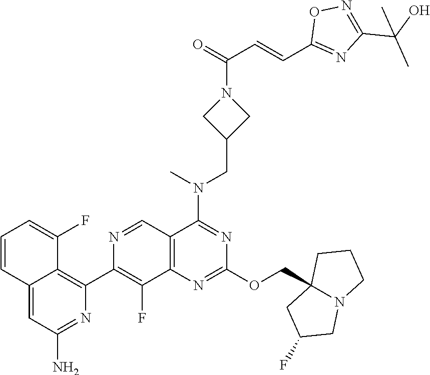 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12]

### Example 35: (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

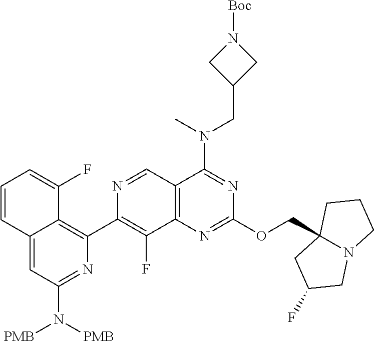

### Step 1: tert-butyl 3-(((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The crude product was purified by column chromatography (silica gel, 100-200 mesh, 80-100% methanol in dichloromethane) affording tert-butyl 3-(((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (240 mg, 35.78%) as a yellow solid. LCMS Rt=0.905 min, m/z=904.4 [M+H]⁺.

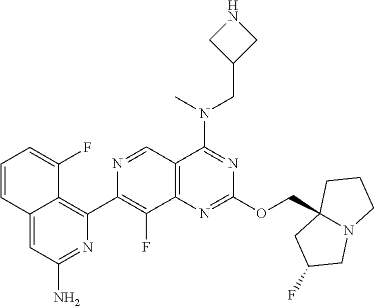 [SMILES: CN(CC1CNC1)c1nc(OC[C@@]23CCCN2C[C@H](F)C3)nc2c(F)c(-c3nc(N)cc4cccc(F)c34)ncc12]

### Step 2: 7-(3-amino-8-fluoroisoquinolin-1-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The reaction mixture was concentrated in vacuo affording 7-(3-amino-8-fluoroisoquinolin-1-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (74 mg, crude, trifluoroacetate salt) as a yellow oil, used in the next step without further purification. LCMS Rt=0.501 min, m/z=564.3 [M+H]⁺.

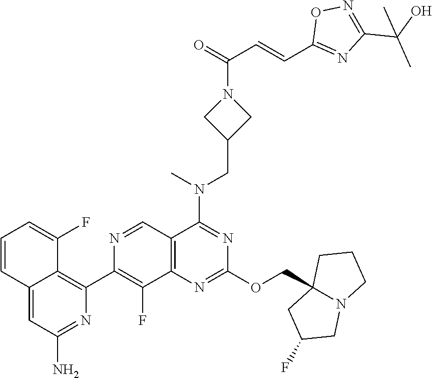 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12]

### Step 3: (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 35%-65%, 8 min) affording (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(2-hydroxypropan-2-yl)-1,2,4-oxadiazol-5-yl)prop-2-en-1-one (19.62 mg, 23.48%) as a white solid: ¹H NMR (400 MHz, Acetonitrile-d3) δ 9.28-9.20 (m, 1H), 7.52-7.43 (m, 2H), 7.37-7.28 (m, 1H), 7.20-7.12 (m, 1H), 6.90 (d, J=2.3 Hz, 1H), 6.86-6.77 (m, 1H), 5.36-5.11 (m, 3H), 4.51-4.42 (m, 1H), 4.31-4.05 (m, 6H), 3.93 (dd, J=5.5, 10.5 Hz, 1H), 3.58 (s, 4H), 3.28-3.17 (m, 1H), 3.16-3.01 (m, 3H), 2.93-2.83 (m, 1H), 2.10-2.01 (m, 3H), 1.90-1.75 (m, 3H), 1.57 (s, 6H). LCMS Rt=2.392 min, m/z=744.3 [M+H]⁺.

LCMS (5 to 95% acetonitrile in water+0.03% ammonium bicarbonate over 6 mins) retention time 2.392 min, ESI+ found [M+H]=744.3.

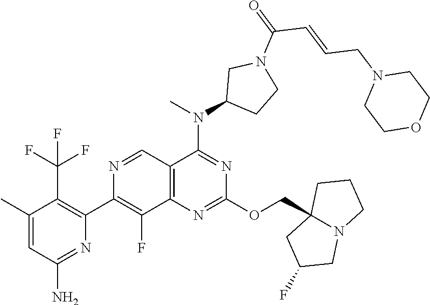 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F]

### Example 36: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one

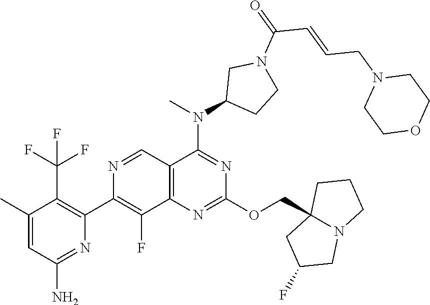 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F]

### Step 1: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one

The Horner-Wadsworth-Emmons reaction was prepared in a similar fashion to Example #2, Step 6. The residue was purified by reverse phase HPLC (column: Waters Xbridge BEH C18 100*30 mm*10 um; mobile phase: [water (NH4HCO3)-acetonitrile]; B %: 25%-50%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one (12.52 mg, 41.77%) as a white solid: ¹H NMR (400 MHz, Acetonitrile-d3) δ 9.10 (d, J=3.1 Hz, 1H), 6.70 (dtd, J=3.1, 6.1, 15.2 Hz, 1H), 6.54 (s, 1H), 6.44-6.29 (m, 1H), 5.44 (br s, 2H), 5.35-5.14 (m, 2H), 4.22-4.14 (m, 1H), 4.12-4.06 (m, 1H), 4.06-3.85 (m, 1H), 3.85-3.71 (m, 1H), 3.64-3.58 (m, 4H), 3.53-3.38 (m, 1H), 3.35 (d, J=2.5 Hz, 3H), 3.12-3.02 (m, 4H), 2.91-2.82 (m, 1H), 2.43-2.32 (m, 7H), 2.27-2.21 (m, 1H), 2.21-2.13 (m, 2H), 2.09-1.98 (m, 2H), 1.90-1.75 (m, 3H). LCMS Rt=0.928 min, m/z=731.3 [M+H]⁺.

LCMS (5 to 95% acetonitrile in water+0.1% ammonium bicarbonate over 6 mins) retention time 0.928 min, ESI+ found [M+H]=731.3.

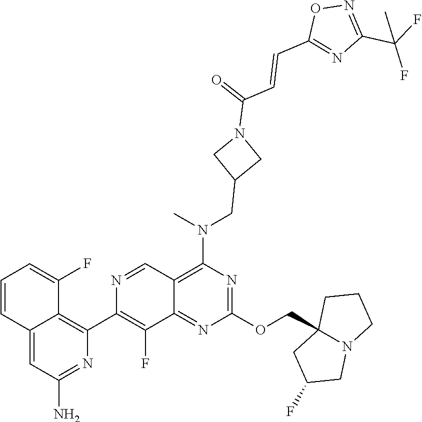 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12]

### Example 37: (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

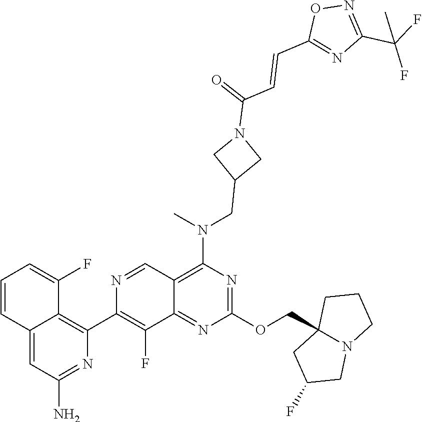 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12]

### Step 1: (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH4HCO3)-acetonitrile]; B %: 35%-65%, 8 min) affording (E)-1-(3-(((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one (4.3 mg, 7.06%) as a yellow amorphous solid: ¹H NMR (400 MHz, Acetonitrile-d3) δ 9.30-9.17 (m, 1H), 7.50-7.44 (m, 2H), 7.41-7.35 (m, 1H), 7.30-7.22 (m, 1H), 6.92-6.87 (m, 1H), 6.86-6.78 (m, 1H), 5.37-5.19 (m, 1H), 5.19-5.10 (m, 2H), 4.53-4.43 (m, 1H), 4.33-4.08 (m, 6H), 3.99-3.90 (m, 1H), 3.61-3.52 (m, 3H), 3.28-3.20 (m, 1H), 3.15 (br d, J=13.6 Hz, 2H), 3.09-3.04 (m, 1H), 2.95-2.83 (m, 1H), 2.23-2.17 (m, 3H), 2.13 (br s, 1H), 2.08 (s, 1H), 2.03 (s, 1H), 1.90-1.75 (m, 3H). LCMS Rt=2.904 min, m/z=750.3 [M+H]⁺.

LCMS (5 to 95% acetonitrile in water+0.03% ammonium bicarbonate over 6 mins) retention time 2.904 min, ESI+ found [M+H]=750.3.

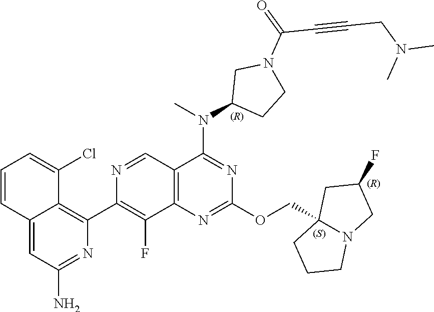

### Example 38: 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one

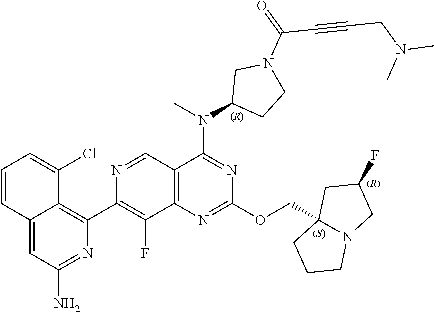

### Step 1: 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH4HCO3)-acetonitrile]; B %: 30%-60%, 8 min) affording 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one (9.68 mg, 14.46%) as a yellow amorphous solid: ¹H NMR (400 MHz, Acetonitrile-d3) δ 9.19-9.13 (m, 1H), 7.67-7.60 (m, 1H), 7.47-7.37 (m, 1H), 7.28-7.20 (m, 1H), 6.95-6.89 (m, 1H), 5.19 (br s, 2H), 5.15 (br s, 2H), 4.25-4.08 (m, 2H), 4.03-3.85 (m, 1H), 3.79-3.64 (m, 2H), 3.52-3.36 (m, 6H), 3.20-3.08 (m, 2H), 3.08-3.04 (m, 1H), 2.94-2.84 (m, 1H), 2.40-2.30 (m, 2H), 2.28 (s, 3H), 2.25-2.22 (m, 3H), 2.12-2.00 (m, 3H), 1.91-1.80 (m, 3H). LCMS Rt=2.665 min, m/z=689.3 [M+H]⁺.

LCMS (5 to 95% acetonitrile in water+0.03% ammonium bicarbonate over 6 mins) retention time 2.665 min, ESI+ found [M+H]=689.3.

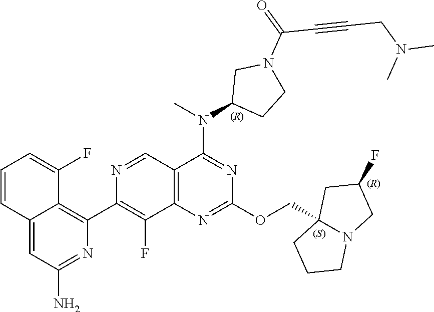

### Example 39: 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one

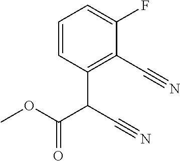 [SMILES: COC(=O)C(C#N)c1cccc(F)c1C#N]

### Step 1: methyl 2-cyano-2-(2-cyano-3-fluorophenyl)acetate

To a solution of 2,6-difluorobenzonitrile (50 g, 359.45 mmol) in dimethyl sulfoxide (250 mL) was added potassium carbonate (99.36 g, 718.90 mmol) and methyl 2-cyanoacetate (35.62 g, 359.45 mmol). The mixture was stirred at 25° C. for 12 h. The resulting solution was diluted with water (1 L). The resulting precipitate was filtered affording methyl 2-cyano-2-(2-cyano-3-fluoro-phenyl)acetate (70 g, crude) as a yellow solid used in the next step without further purification.

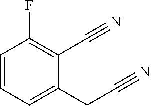 [SMILES: N#CCc1cccc(F)c1C#N]

### Step 2: 2-(cyanomethyl)-6-fluoro-benzonitrile

A solution of methyl 2-cyano-2-(2-cyano-3-fluoro-phenyl)acetate (69 g, 316.25 mmol) in dimethyl sulfoxide (400 mL) and hydrochloric acid (6 M, 138.00 mL) was stirred at 70° C. for 12 h. Then the resulting solution was adjusted to pH=9.0 with saturated sodium carbonate. The resulting precipitate was filtered and dried affording 2-(cyanomethyl)-6-fluoro-benzonitrile (25 g, crude) as a white solid used in the next step without further purification: ¹H NMR (400 MHz, dimethylsulfoxide-d6) δ 7.85 (td, J=8.13, 6.13 Hz, 1H), 7.44-7.65 (m, 2H), 4.34 (s, 2H).

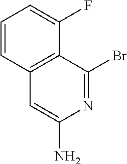 [SMILES: Nc1cc2cccc(F)c2c(Br)n1]

### Step 3: 1-bromo-8-fluoro-isoquinolin-3-amine

A solution of 2-(cyanomethyl)-6-fluoro-benzonitrile (25 g, 156.11 mmol) in hydrogen bromide (248.33 g, 920.75 mmol, 30% purity) was stirred for 30 min at 0° C. The resulting solution was adjusted to pH=8.0 with saturated sodium carbonate (600 mL). The resulting precipitate was filtered and dried affording 1-bromo-8-fluoro-isoquinolin-3-amine (16.5 g, crude) as a yellow oil used in the next step without further purification.

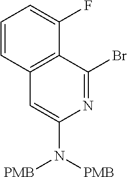

### Step 4: 1-bromo-8-fluoro-N,N-bis[(4-methoxyphenyl)methyl]isoquinolin-3-amine

The PMB protection was prepared in a similar fashion to Example #71, Step 1. The crude product was purified by column chromatography (silica gel, 100-200 mesh, 0-100% ethyl acetate in petroleum ether) affording 1-bromo-8-fluoro-N,N-bis[(4-methoxyphenyl)methyl]isoquinolin-3-amine (11.5 g, 40%) as a yellow solid: ¹H NMR (400 MHz, Dimethylsulfoxide-d6) δ 7.36-7.50 (m, 2H), 7.22 (br d, J=7.63 Hz, 4H), 6.95-7.05 (m, 1H), 6.82-6.93 (m, 5H), 4.65-4.80 (m, 4H), 3.66-3.78 (m, 6H). LCMS Rt=0.519 min, m/z=480.1 [M+H]⁺.

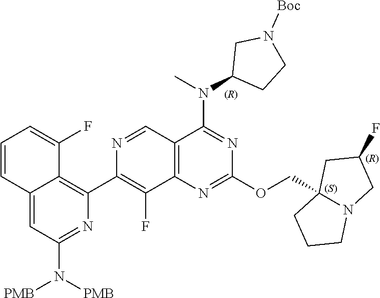

### Step 5: (R)-tert-butyl 3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The residue was purified by column chromatography (silica gel, 100-200 mesh, 0-100% ethyl acetate in petroleum ether) affording (R)-tert-butyl 3-((7-(3-(bis(4-methoxybenzyl)amino)-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (1.5 g, 73.08%) as a brown oil. LCMS Rt=2.675 min, m/z=904.4 [M+H]⁺.

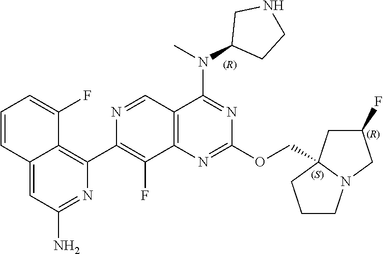

### Step 6: 7-(3-amino-8-fluoroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The de-Boc and PMB protecting group was prepared in a similar fashion to Example #71, Step 7. The residue was purified by reverse phase HPLC (column: Phenomenex Luna 80*30 mm*3 um; mobile phase: [water (trifluroacetic acid)-acetoniitrile]; B %: 1%-30%, 8 min) affording 7-(3-amino-8-fluoroisoquinolin-1-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (450 mg, 85.73%, trifluoroacetic salt) as a yellow solid. LCMS Rt=0.455 min, m/z=564.3 [M+H]⁺.

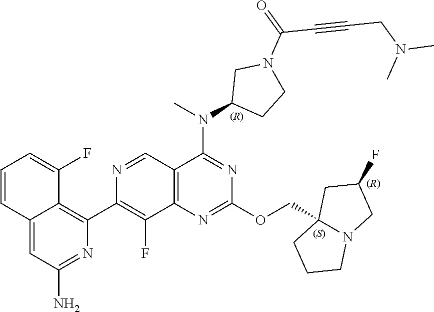

### Step 7: 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH4HCO3)-acetonitrile]; B %: 20%-50%, 8 min) affording 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-yn-1-one (1.69 mg, 99.76%) as a white solid: ¹H NMR (400 MHz, Acetonitrile-d3) δ 9.21 (d, J=4.4 Hz, 1H), 7.52-7.46 (m, 2H), 6.94-6.81 (m, 2H), 5.36 (br s, 1H), 5.26-5.18 (m, 2H), 4.30-4.12 (m, 3H), 4.05-3.87 (m, 1H), 3.81-3.65 (m, 2H), 3.53-3.46 (m, 2H), 3.43 (d, J=6.9 Hz, 4H), 3.22-3.15 (m, 2H), 3.14-3.09 (m, 1H), 2.97-2.89 (m, 1H), 2.31 (s, 6H), 2.25-2.19 (m, 2H), 2.16-2.05 (m, 3H), 1.93-1.84 (m, 3H). LCMS Rt=1.811 min, m/z=673.3 [M+H]⁺.

LCMS (5 to 95% acetonitrile in water+0.03% ammonium bicarbonate over 6 mins) retention time 1.811 min, ESI+ found [M+H]=673.3.

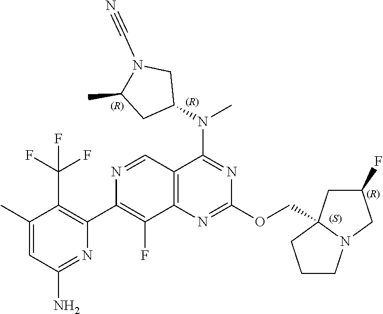

### (2R,4R)-4-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carbonitrile

### Example 40: (2R,4R)-4-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carbonitrile

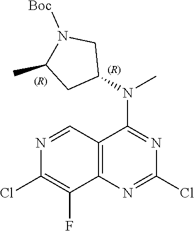

### Step 1: (2R,4R)-tert-butyl 4-((2,7-dichloro-8-fluoropyrido[4,3-d]pyrimidin-4-yl)(methyl)amino)-2-methylpyrrolidine-1-carboxylate

The substitution reaction was prepared in a similar fashion to Example #71, Step 3. The mixture was concentrated in vacuo affording (2R,4R)-tert-butyl 4-((2,7-dichloro-8-fluoropyrido[4,3-d]pyrimidin-4-yl)(methyl)amino)-2-methylpyrrolidine-1-carboxylate (4.1 g, crude) as a brown oil, used in the next step without further purification. LCMS Rt=0.876 min, m/z=429.1 [M+H]⁺.

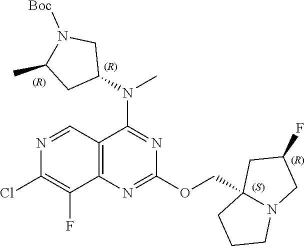

### Step 2: (2R,4R)-tert-butyl 4-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carboxylate

The substitution reaction was prepared in a similar fashion to Example #71, Step 4. The mixture was diluted with water (30 mL) and extracted with ethyl acetate (3×50 mL). The combined organic layers were dried over sodium sulphate and concentrated in vacuo affording (2R,4R)-tert-butyl 4-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carboxylate (4.1 g, crude) as a brown oil, used in the next step without further purification. LCMS Rt=1.006 min, m/z=552.2 [M+H]⁺.

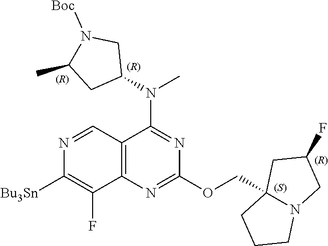

### Step 3: (2R,4R)-tert-butyl 4-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)-2-methylpyrrolidine-1-carboxylate

The tin reagent formation was prepared in a similar fashion to Example #71, Step 5. The reaction mixture was quenched with saturated potassium fluoride (30 mL) at 0° C. and extracted with dichloromethane (3×100 mL). The combined organic layers were dried over sodium sulphate and concentrated in vacuo affording (2R,4R)-tert-butyl 4-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)-2-methylpyrrolidine-1-carboxylate (500 mg, crude) as a green oil, used in the next step without further purification. LCMS Rt=0.677 min, m/z=808.4 [M+H]⁺.

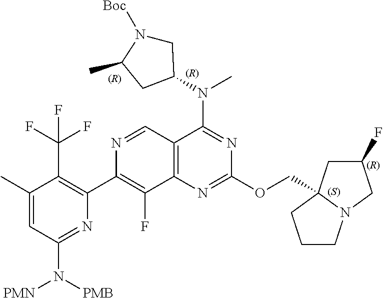

### Step 4: (2R,4R)-tert-butyl 4-((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carboxylate

The Stille reaction was prepared in a similar fashion to Example #71, Step 6. The mixture was purified by reverse phase HPLC(column: Phenomenex Luna 80*30 mm*3 um; mobile phase: [water (trifluroacetic acid)-acetoniitrile]; B %: 35%-75%, 8 min) affording (2R,4R)-tert-butyl 4-((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carboxylate (100 mg, 17.31%, trifluoroacetate salt) as a yellow oil. LCMS Rt=2.247 min, m/z=932.4 [M+H]⁺.

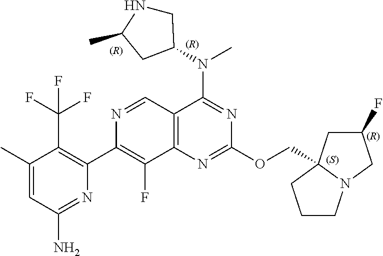

### Step 5: 7-(6-amino-4-methyl-3-(trifluoromethyl)pyridin-2-yl)-8-fluoro-2-(((2R,7aS)-2-fluorohexahydro-1H-pyrrolizin-7a-yl)methoxy)-N-methyl-N-((3R,5R)-5-methylpyrrolidin-3-yl)pyrido[4,3-d]pyrimidin-4-amine

The deprotection of Boc and PMB group was prepared in a similar fashion to Example #71, Step 7. The mixture was concentrated in vacuo affording 7-(6-amino-4-methyl-3-(trifluoromethyl)pyridin-2-yl)-8-fluoro-2-(((2R,7aS)-2-fluorohexahydro-1H-pyrrolizin-7a-yl)methoxy)-N-methyl-N-((3R,5R)-5-methylpyrrolidin-3-yl)pyrido[4,3-d]pyrimidin-4-amine (100 mg, crude, trifluoroacetate salt) as a brown oil, used in the next step without further purification. LCMS Rt=0.667 min, m/z=592.3 [M+H]⁺.

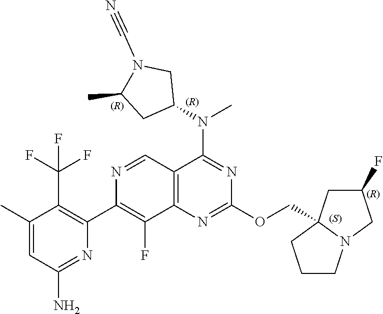

### Step 6: (2R,4R)-4-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carbonitrile

The substitution reaction was prepared in a similar fashion to Example 13, Step 6. The crude product was purified by reverse phase HPLC (column: Waters Xbridge Prep OBD C18 150*40 mm*10 um; mobile phase: [water (NH4HCO3)-acetonitrile]; B %: 25%-50%, 8 min) affording (2R,4R)-4-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)-2-methylpyrrolidine-1-carbonitrile (3.77 mg, 3.54%) as a pale yellow oil: ¹H NMR (400 MHz, Acetonitrile-d3) δ 9.17-9.08 (m, 1H), 6.56 (br s, 1H), 5.58-5.42 (m, 2H), 5.40-5.19 (m, 2H), 4.23-4.12 (m, 1H), 4.07-3.86 (m, 2H), 3.58-3.51 (m, 1H), 3.46-3.36 (m, 3H), 3.35-3.26 (m, 1H), 3.20-3.06 (m, 2H), 2.96-2.86 (m, 1H), 2.69-2.58 (m, 1H), 2.43 (br s, 3H), 2.11 (br s, 2H), 2.07-1.97 (m, 4H), 1.92-1.79 (m, 2H), 1.45-1.28 (m, 3H). LCMS Rt=2.849 min, m/z=617.3 [M+H]⁺.

LCMS (5 to 95% acetonitrile in water+0.03% ammonium bicarbonate over 6 mins)) retention time 2.849 min, ESI+ found [M+H]=617.3.

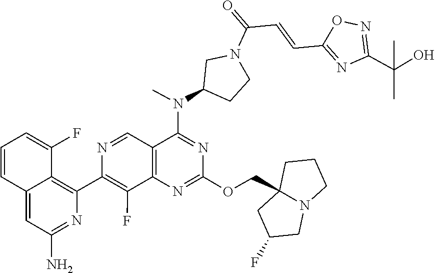 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1]

### Example 41: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

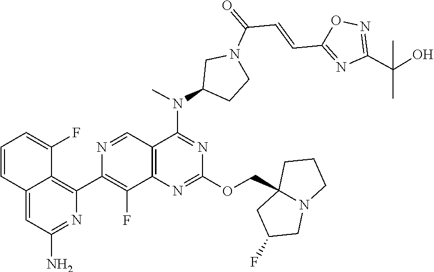 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1]

### Step 1: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one

The amide coupling reaction was prepared in a similar fashion to Example #71, Step 11. The residue was purified by reverse phase HPLC (column: Waters Xbridge Prep OBD C18 150*40 mm*10 um; mobile phase:[water (NH4HCO3)-acetonitrile]; B %: 25%-55%, 8 min) affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(2-hydroxypropan-2-yl)-1,2,4-oxadiazol-5-yl)prop-2-en-1-one (3.38 mg, 6.16%) as a yellow amorphous solid: ¹H NMR (400 MHz, Acetonitrile-d3) δ 9.23 (s, 1H), 7.54-7.36 (m, 4H), 6.93 (d, J=2.5 Hz, 1H), 6.88-6.81 (m, 1H), 5.48-5.38 (m, 1H), 5.37 (br d, J=7.9 Hz, 1H), 5.21 (br s, 1H), 4.30-4.19 (m, 2H), 4.04 (br dd, J=8.2, 12.4 Hz, 1H), 3.96-3.85 (m, 1H), 3.84-3.74 (m, 1H), 3.69-3.59 (m, 1H), 3.54 (td, J=8.6, 12.6 Hz, 1H), 3.45 (s, 3H), 3.27-3.09 (m, 3H), 2.99-2.88 (m, 1H), 2.44 (q, J=8.6 Hz, 1H), 2.27-2.21 (m, 2H), 2.14-2.06 (m, 2H), 1.90 (br dd, J=5.5, 11.6 Hz, 3H), 1.60 (d, J=8.4 Hz, 6H). LCMS Rt=3.178 min, m/z=744.3 [M+H]⁺.

LCMS (5 to 95% acetonitrile in water+0.03% ammonium bicarbonate over 6 mins) retention time 3.178 min, ESI+ found [M+H]=744.3

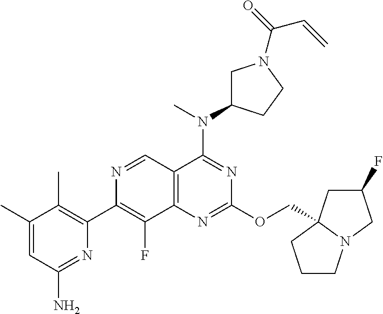 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1]

### Example 42: 1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

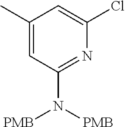

### Step 1: 6-chloro-N,N-bis(4-methoxybenzyl)-4-methylpyridin-2-amine

The PMB protection reaction was prepared in a similar fashion to Example #71, Step 1. The organic layers were concentrated in vacuo affording 6-chloro-N,N-bis(4-methoxybenzyl)-4-methylpyridin-2-amine (50 g, crude) as a yellow solid used in next step without further purification. LCMS Rt=0.915 min, m/z=382.1 [M+H]⁺.

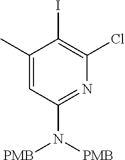

### Step 2: 6-chloro-5-iodo-N,N-bis(4-methoxybenzyl)-4-methylpyridin-2-amine

To a solution of 6-chloro-N,N-bis[(4-methoxyphenyl)methyl]-4-methyl-pyridin-2-amine (10 g, 26.12 mmol) in N,N-dimethyl-formamide (10 mL) was added N-iodo-succinimide (5.88 g, 26.12 mmol), the mixture was stirred at 25° C. for 3 h. The mixture was diluted with water (20 mL), extracted with petroleum ether (20 mL×3). The combined organic layers were concentrated to dryness in vacuo affording 6-chloro-5-iodo-N,N-bis(4-methoxybenzyl)-4-methylpyridin-2-amine (11 g, crude) as a yellow solid used in next step without further purification. LCMS Rt=1.048 min, m/z=508.0 [M+H]⁺.

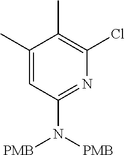

### Step 3: 6-chloro-N,N-bis(4-methoxybenzyl)-4,5-dimethylpyridin-2-amine

To a solution of 6-chloro-5-iodo-N,N-bis[(4-methoxyphenyl)methyl]-4-methyl-pyridin-2-amine (3 g, 5.90 mmol) in dioxane (30 mL) and water (3 mL) was added methylboronic acid (423.56 mg, 7.08 mmol), (1,1′-bis(diphenylphosphino)ferrocene)palladium(II) dichloride (431.45 mg, 589.65 umol), calcium carbonate (5.76 g, 17.69 mmol), the mixture was stirred at 100° C. for 12 h. The mixture was diluted with water (30 mL), extracted with ethyl acetate (20 mL×3). The combined organic layers were concentrated to dryness in vacuo and purified by column chromatography (silica gel, 100-200 mesh, 0-5% ethyl acetate in petroleum ether) affording 6-chloro-N,N-bis(4-methoxybenzyl)-4,5-dimethylpyridin-2-amine (1.6 g, 68.37%) as a white solid. LCMS Rt=1.034 min, m/z=396.2 [M+H]⁺.

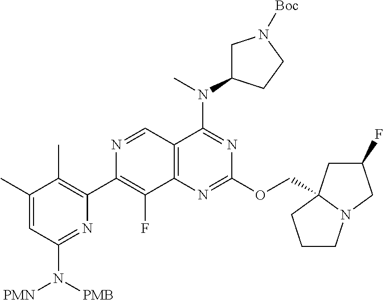

### Step 4: (R)-tert-butyl 3-((7-(6-(bis(4-methoxybenzyl)amino)-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. the residue was purified by reverse phase HPLC(column: Phenomenex luna C18 250*50 mm*10 um; mobile phase: [water (trifluroacetic acid)-acetoniitrile]; B %: 40%-70%, 10 min) affording (R)-tert-butyl 3-((7-(6-(bis(4-methoxybenzyl)amino)-3,4-dimethylpyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (320 mg, 36.70%) as a yellow solid. LCMS Rt=0.796 min, m/z=864.5 [M+H]⁺.

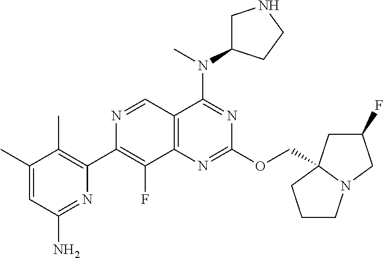 [SMILES: Cc1cc(N)nc(-c2ncc3c(N(C)[C@@H]4CCNC4)nc(OC[C@@]45CCCN4C[C@H](F)C5)nc3c2F)c1C]

### Step 5: 7-(6-amino-3,4-dimethylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and PMB group was prepared in a similar fashion to Example #71, Step 7. The reaction mixture was concentrated in vacuo affording 7-(6-amino-3,4-dimethylpyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (100 mg, crude, trifluoroacetic acid salt) as a brown oil, used in next step without further purification. LCMS Rt=0.468 min, m/z=524.3 [M+H]⁺.

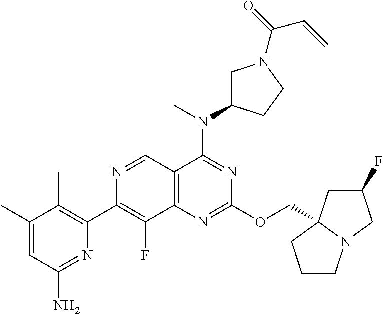 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1]

### Step 6: 1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 30%-60%, 10 min) affording 1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (7.5 mg, 11.04%) as a yellow oil: ¹H NMR (400 MHz, acetonitrile-d3) δ 9.17 (s, 1H), 8.18 (s, 1H), 6.63-6.50 (m, 1H), 6.49 (s, 1H), 6.27-6.17 (m, 1H), 5.71-5.61 (m, 1H), 5.42-5.22 (m, 2H), 4.36-4.25 (m, 2H), 3.92 (dd, J=8.1, 12.8 Hz, 1H), 3.88-3.75 (m, 1H), 3.62 (br s, 1H), 3.57-3.42 (m, 2H), 3.38 (s, 3H), 3.36 (br s, 1H), 3.34-3.16 (m, 2H), 2.99 (br d, J=5.6 Hz, 1H), 2.40-2.25 (m, 3H), 2.24 (s, 3H), 2.21 (br s, 1H), 2.19-2.10 (m, 1H), 2.03-1.94 (m, 3H), 1.93-1.85 (m, 3H). LCMS Rt=2.497 min, m/z=578.3 [M+H]⁺.

LCMS (5 to 95% acetonitrile in water+0.1% ammonium bicarbonate over 6 mins) retention time 2.497 min, ESI+ found [M+H]=578.3.

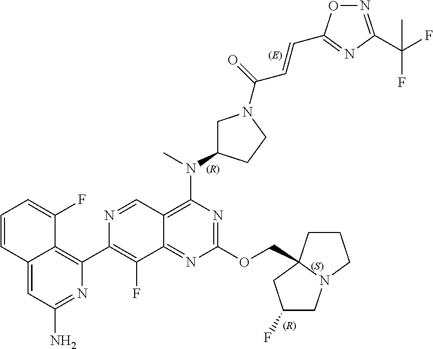

### Example 43: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one

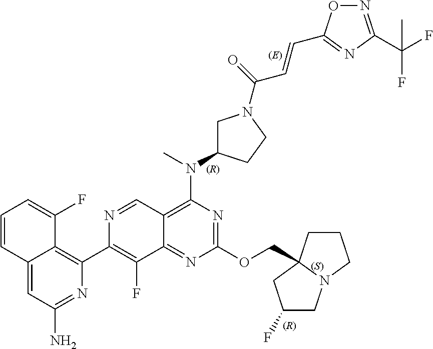

### Step 1: (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one

The amide coupling reaction was prepared in a similar fashion to Example #71, Step 11. The residue was purified by reverse phase HPLC (column: Phenomenex Luna C18 75*30 mm*3 um; mobile phase: [water(FA)-acetonitrile]; B %: 5%-35%, 8 min) affording (E)-1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one (1.3 mg, 1.84%, formate salt) as a yellow oil: ¹H NMR (400 MHz, Acetonitrile-d3) δ 9.28-9.21 (m, 1H), 7.67-7.42 (m, 4H), 6.96-6.80 (m, 2H), 5.48-5.33 (m, 2H), 5.24-5.13 (m, 2H), 4.30-4.17 (m, 3H), 4.05 (br s, 1H), 3.95-3.76 (m, 2H), 3.69-3.47 (m, 2H), 3.46 (s, 3H), 3.27-3.09 (m, 4H), 2.99-2.90 (m, 1H), 2.45 (br d,J=8.3 Hz, 1H), 2.35 (br d, J=8.3 Hz, 1H), 2.11 (s, 2H), 2.07 (br d, J=7.9 Hz, 2H), 1.91 (br dd,J=5.3, 11.5 Hz, 2H). LCMS Rt=2.185 min, m/z=750.3 [M+H]⁺.

LCMS (5 to 95% acetonitrile in water+0.1% formate acid over 6 mins) retention time 2.185 min, ESI+ found [M+H]=750.3.

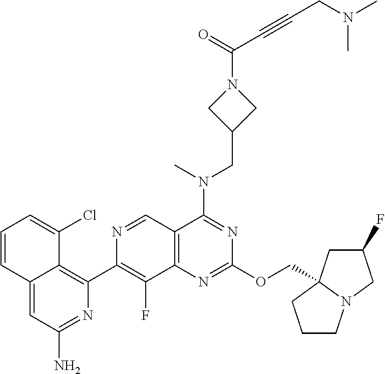 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1]

### Example 44: 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-(dimethylamino)but-2-yn-1-one

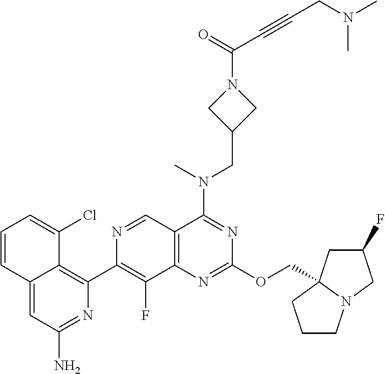 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1]

### Step 1: 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-(dimethylamino)but-2-yn-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH4HCO3)-acetonitrile]; B %: 25%-55%, 8 min) affording 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-4-(dimethylamino)but-2-yn-1-one (11.7 mg, 12.48%) as a yellow amorphous solid: ¹H NMR (400 MHz, Acetonitrile-d3) δ 8.92 (dd, J=1.9, 5.2 Hz, 1H), 7.62 (d, J=8.4 Hz, 1H), 7.44-7.36 (m, 1H), 7.23 (d, J=7.3 Hz, 1H), 6.87 (s, 1H), 5.36-5.10 (m, 3H), 4.23-4.02 (m, 3H), 3.99-3.80 (m, 2H), 3.77-3.63 (m, 2H), 3.55-3.36 (m, 3H), 3.30-3.22 (m, 2H), 3.14-3.04 (m, 3H), 2.96-2.87 (m, 3H), 2.16 (br d, J=1.3 Hz, 6H), 2.11-2.00 (m, 3H), 1.90-1.76 (m, 3H). LCMS Rt=2.474 min, m/z=689.3 [M+H]⁺.

LCMS (5 to 95% acetonitrile in water+0.03% ammonium bicarbonate over 6 mins) retention time 2.474 min, ESI+ found [M+H]=689.3.

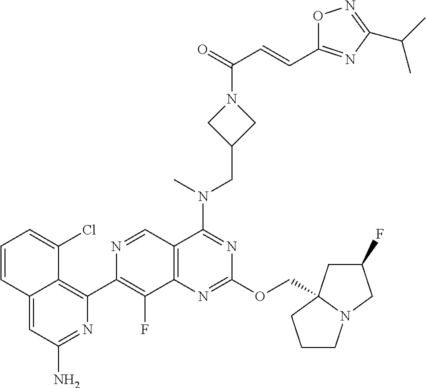 [SMILES: CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1]

### Example 45: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

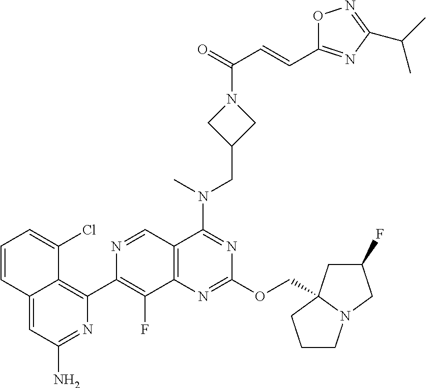 [SMILES: CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1]

### Step 1: (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH4HCO3)-acetonitrile]; B %: 30%-60%, 8 min) affording (E)-1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one (30.1 mg, 46.67%) as a yellow amorphous solid: ¹H NMR (400 MHz, Chloroform-d) δ 9.22-9.17 (m, 1H), 7.57-7.47 (m, 2H), 7.37 (t, J=7.9 Hz, 1H), 7.25 (d, J=7.3 Hz, 1H), 7.10 (br d, J=15.6 Hz, 1H), 6.88 (s, 1H), 5.38-5.19 (m, 1H), 4.65 (s, 2H), 4.52 (br s, 1H), 4.37-4.01 (m, 7H), 3.61 (s, 3H), 3.30-3.21 (m, 3H), 3.15 (br d, J=6.9 Hz, 2H), 2.98 (br s, 1H), 2.28-2.11 (m, 3H), 1.93 (br s, 3H), 1.37 (d, J=7.0 Hz, 6H). LCMS Rt=2.759 min, m/z=744.3 [M+H]⁺.

LCMS (5 to 95% acetonitrile in water+0.03% ammonium bicarbonate over 6 mins)) retention time 2.759 min, ESI+ found [M+H]=744.3.

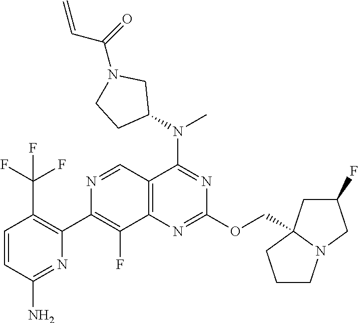 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1]

### Example 46: 1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

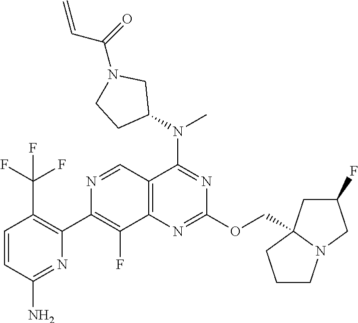 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1]

### Step 1: 1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 30%-70%, 8 min) affording 1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (9.19 mg, 27.95%) as a yellow solid: ¹H NMR (400 MHz, Acetonitrile-d3) δ 9.17 (d, J=2.0 Hz, 1H), 7.84 (d, J=8.9 Hz, 1H), 6.73 (d, J=8.8 Hz, 1H), 6.60 (dt, J=10.4, 16.8 Hz, 1H), 6.31-6.18 (m, 1H), 5.76-5.65 (m, 1H), 5.60 (br s, 2H), 5.46-5.17 (m, 2H), 4.25-4.12 (m, 2H), 4.08-3.92 (m, 1H), 3.90-3.78 (m, 1H), 3.73-3.61 (m, 1H), 3.60-3.44 (m, 1H), 3.41 (s, 3H), 3.23-3.04 (m, 3H), 2.98-2.86 (m, 1H), 2.45-2.27 (m, 2H), 2.25-2.19 (m, 1H), 2.13-2.04 (m, 2H), 1.95-1.83 (m, 3H). LCMS Rt=2.421 min, m/z=618.3 [M+H]⁺.

LCMS (5 to 95% acetonitrile in water+0.1% ammonium bicarbonate over 6 mins) retention time 2.421 min, ESI+ found [M+H]=618.3.

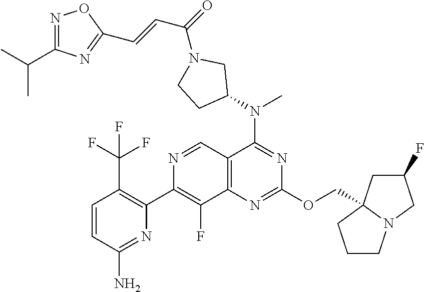 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1]

### Example 47: (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

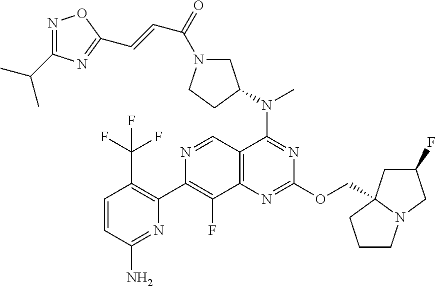 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1]

### Step 1: (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 35%-65%, 8 min) affording (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (7.98 mg, 20.20%) as a yellow solid: ¹H NMR (400 MHz, Acetonitrile-d3) δ 9.18 (d, J=2.3 Hz, 1H), 7.84 (d, J=8.9 Hz, 1H), 7.52-7.32 (m, 2H), 6.73 (d, J=8.8 Hz, 1H), 5.60 (s, 2H), 5.47-5.17 (m, 2H), 4.27-4.22 (m, 1H), 4.22-4.12 (m, 1H), 4.02 (br dd, J=8.1, 12.8 Hz, 1H), 3.93-3.85 (m, 1H), 3.82-3.74 (m, 1H), 3.66-3.49 (m, 1H), 3.43 (d, J=1.0 Hz, 3H), 3.22-3.07 (m, 4H), 2.97-2.87 (m, 1H), 2.48-2.40 (m, 1H), 2.38-2.29 (m, 1H), 2.27-2.21 (m, 1H), 2.13 (br s, 1H), 2.07 (br d, J=5.3 Hz, 1H), 1.93-1.78 (m, 3H), 1.35 (dd, J=7.0, 8.0 Hz, 6H). LCMS Rt=2.743 min, m/z=728.3 [M+H]⁺.

LCMS (5 to 95% acetonitrile in water+0.1% ammonium bicarbonate over 6 mins) retention time 2.743 min, ESI+ found [M+H]=728.3.

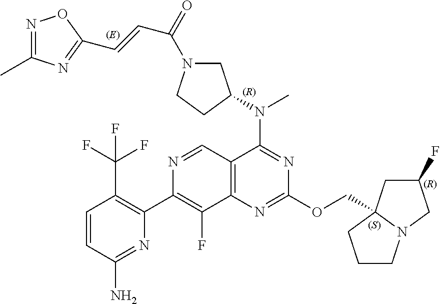

### Example 48: (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

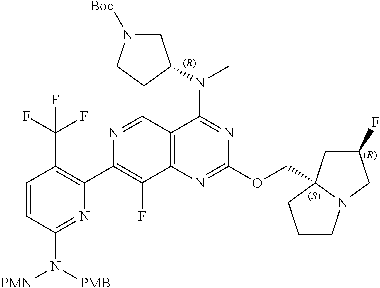

### Step 1: (R)-tert-butyl 3-((7-(6-(bis(4-methoxybenzyl)amino)-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The resulting residue was purified by column chromatography (silica gel, 100-200 mesh, 100% ethyl acetate in petroleum ether) affording (R)-tert-butyl 3-((7-(6-(bis(4-methoxybenzyl)amino)-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (800 mg, 77.95%) as a yellow oil. LCMS Rt=0.780 min, m/z=904.4 [M+H]⁺.

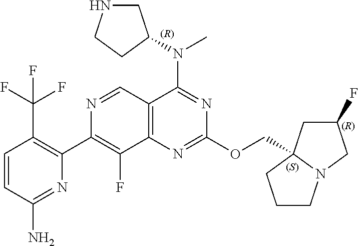

### Step 2: 7-(6-amino-3-(trifluoromethyl)pyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

The deprotection of Boc and PMB group was prepared in a similar fashion to Example #71, Step 7. The reaction mixture was concentrated in vacuo affording 7-(6-amino-3-(trifluoromethyl)pyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (140 mg, 37.34%) as a white solid. LCMS Rt=0.991 min, m/z=564.2 [M+H]⁺.

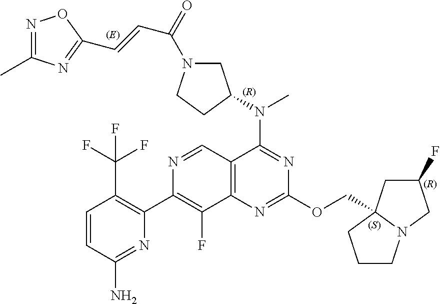

### Step 3: (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #71, Step 11. The residue was purified by reverse phase HPLC (column: Waters Xbridge BEH C18 100*30 mm*10 um; mobile phase: [water (NH4HCO3)-acetonitrile]; B %: 5%-55%, 8 min) affording (E)-1-((R)-3-((7-(6-amino-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one (15.29 mg, 40.71%) as a yellow solid. ¹H NMR (400 MHz, Acetonitrile-d3) δ 9.17 (d, J=1.3 Hz, 1H), 7.84 (d, J=8.9 Hz, 1H), 7.53-7.32 (m, 2H), 6.73 (d, J=8.8 Hz, 1H), 5.62 (br s, 2H), 5.47-5.14 (m, 2H), 4.26-4.19 (m, 1H), 4.18-4.13 (m, 1H), 4.02 (br dd, J=8.0, 12.8 Hz, 1H), 3.95-3.70 (m, 2H), 3.66-3.47 (m, 1H), 3.42 (s, 3H), 3.23-3.11 (m, 2H), 3.10-3.03 (m, 1H), 2.98-2.84 (m, 1H), 2.46-2.39 (m, 4H), 2.37-2.30 (m, 1H), 2.25 (br s, 1H), 2.14-2.06 (m, 2H), 1.94-1.79 (m, 3H). LCMS Rt=2.530 min, m/z=700.3 [M+H]⁺.

LCMS (5 to 95% acetonitrile in water+0.03% ammonium bicarbonate over 6 min) retention time 2.530 min, ESI+ found [M+H]=700.3.

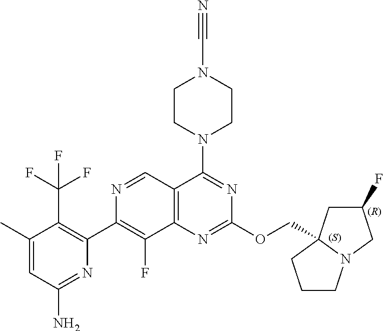

### Example 49: 4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazine-1-carbonitrile

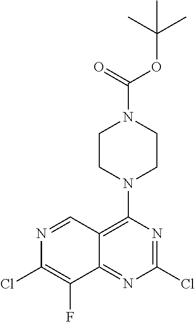 [SMILES: CC(C)(C)OC(=O)N1CCN(c2nc(Cl)nc3c(F)c(Cl)ncc23)CC1]

### Step 1: tert-butyl 4-(2,7-dichloro-8-fluoro-pyrido[4,3-d]pyrimidin-4-yl)piperazine-1-carboxylate

The substitution reaction was prepared in a similar fashion to Example #71, Step 3. The residue was purified by column chromatography (silica gel, 100-200 mesh, 0-100% ethyl acetate in petroleum ether)affording tert-butyl 4-(2,7-dichloro-8-fluoro-pyrido[4,3-d]pyrimidin-4-yl)piperazine-1-carboxylate (2.2 g, 46.03%) as a brown solid. LCMS Rt=0.699 min, m/z=401.1 [M+H]⁺.

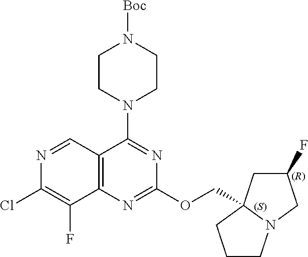

### Step 2: tert-butyl 4-(7-chloro-8-fluoro-2-(((2R,7aS)-2-fluorohexahydro-1H-pyrrolizin-7a-yl)methoxy)pyrido[4,3-d]pyrimidin-4-yl)piperazine-1-carboxylate

The substitution reaction was prepared in a similar fashion to Example #71, Step 4. The crude product was purified by column chromatography (silica gel, 100-200 mesh, 80-100% ethyl acetate in petroleum ether) affording tert-butyl 4-(7-chloro-8-fluoro-2-(((2R,7aS)-2-fluorohexahydro-1H-pyrrolizin-7a-yl)methoxy)pyrido[4,3-d]pyrimidin-4-yl)piperazine-1-carboxylate (1.7 g, 68.56%) as a yellow solid. LCMS Rt=0.540 min, m/z=524.2 [M+H]⁺.

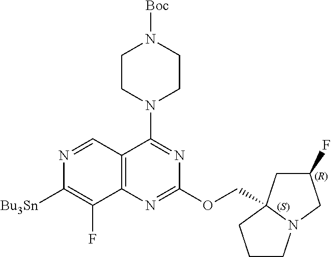

### Step 3: tert-butyl 4-(8-fluoro-2-(((2R,7aS)-2-fluorohexahydro-1H-pyrrolizin-7a-yl)methoxy)-7-(tributylstannyl)pyrido[4,3-d]pyrimidin-4-yl)piperazine-1-carboxylate

The tin reagent formation was prepared in a similar fashion to Example #71, Step 5. The mixture was purified by column chromatography (silica gel, 100-200 mesh, 80-100% tetrahydrofuran in petroleum ether) affording tert-butyl 4-(8-fluoro-2-(((2R,7aS)-2-fluorohexahydro-1H-pyrrolizin-7a-yl)methoxy)-7-(tributylstannyl)pyrido[4,3-d]pyrimidin-4-yl)piperazine-1-carboxylate (320 mg, 53.87%) as a yellow oil. LCMS Rt=0.663 min, m/z=780.4 [M+H]⁺.

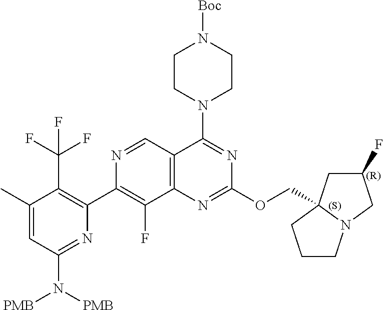

### Step 4: tert-butyl 4-(7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate

The Stille reaction was prepared in a similar fashion to Example #71, Step 6. The residue was purified by column chromatography (silica gel, 100-200 mesh, 0-100% ethyl acetate in petroleum ether) affording tert-butyl 4-(7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazine-1-carboxylate (300 mg, 95.72%) as a yellow oil. LCMS Rt=0.701, m/z=904.4 [M+H]⁺.

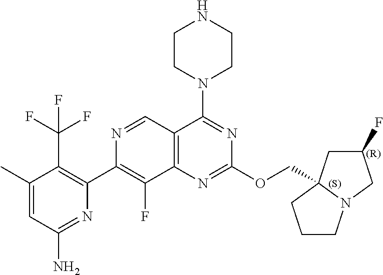

### Step 5: 6-(8-fluoro-2-(((2R,7aS)-2-fluorohexahydro-1H-pyrrolizin-7a-yl)methoxy)-4-(piperazin-1-yl)pyrido[4,3-d]pyrimidin-7-yl)-4-methyl-5-(trifluoromethyl)pyridin-2-amine

The deprotection of Boc and PMB group was prepared in a similar fashion to Example #71, Step 7. The crude product was purified by reverse phase HPLC (column: Phenomenex Luna 80*30 mm*3 um; mobile phase: [water (trifluroacetic acid)-acetoniitrile]; B %: 1%-40%, 8 min) affording 6-(8-fluoro-2-(((2R,7aS)-2-fluorohexahydro-1H-pyrrolizin-7a-yl)methoxy)-4-(piperazin-1-yl)pyrido[4,3-d]pyrimidin-7-yl)-4-methyl-5-(trifluoromethyl)pyridin-2-amine (50 mg, 41.68%, trifluoroacetic salt) as a yellow solid. LCMS Rt=0.436 min, m/z=564.2 [M+H]⁺.

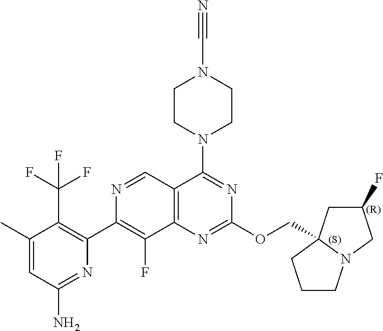

### Step 6: 4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazine-1-carbonitrile

The substitution reaction was prepared in a similar fashion to Example #13, Step 6. The crude was purified by reverse phase HPLC(column: Waters Xbridge BEH C18 100*30 mm*10 um; mobile phase: [water (NH4HCO3)-acetonitrile]; B %: 30%-60%, 8 min) affording 4-(7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)piperazine-1-carbonitrile (11.06 mg, 55.62%) as a yellow solid: ¹H NMR (400 MHz, Acetonitrile-d3) δ 8.98 (s, 1H), 6.56 (s, 1H), 5.43 (s, 2H), 5.19 (br s, 1H), 4.23-4.18 (m, 1H), 4.14-4.09 (m, 1H), 4.03-3.96 (m, 4H), 3.47-3.40 (m, 4H), 3.19-3.11 (m, 2H), 3.08-3.04 (m, 1H), 2.94-2.85 (m, 1H), 2.43 (d, J=1.6 Hz, 3H), 2.18-2.09 (m, 4H), 1.90-1.83 (m, 2H). LCMS Rt=2.554 min, m/z=589.2 [M+H]⁺.

LCMS (5 to 95% acetonitrile in water+0.1% trifluoroacetic acid over 6 mins) retention time 2.554 min, ESI+ found [M+H]=589.2.

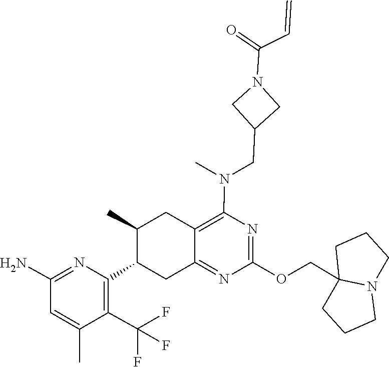 [SMILES: C=CC(=O)N1CC(CN(C)c2nc(OCC34CCCN3CCC4)nc3c2C[C@H](C)[C@@H](c2nc(N)cc(C)c2C(F)(F)F)C3)C1]

### Example 50:1-(3-((((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

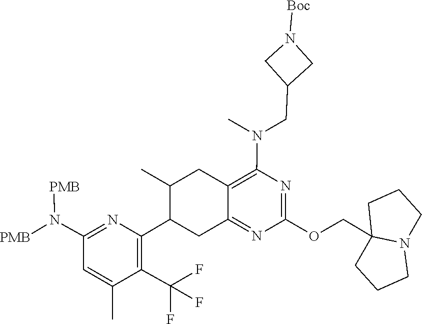

### Step 1: tert-butyl 3-(((7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate

The substitution reaction was prepared in a similar fashion to Example #65, Step 14. The resulting residue was purified by column chromatography (silica gel, 100-200 mesh, 0-100% ethyl acetate in petroleum ether) affording tert-butyl 3-(((7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate (300 mg, 69.9%) as a white solid. LCMS Rt=0.667 min, m/z=899.5 [M+H]⁺.

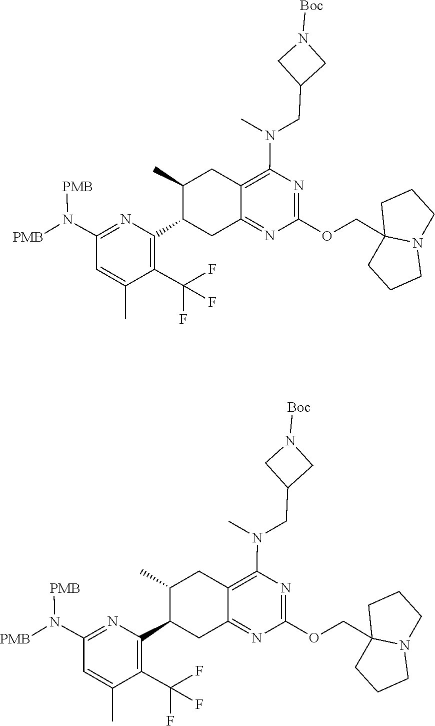

### Step 2: tert-butyl 3-((((6S,7S)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate and tert-butyl 3-((((6R,7R)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate

The mixture of diastereoisomers was separated by SFC to give arbitrarily assigned: tert-butyl 3-((((6S,7S)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate (Peak 1, retention time=2.025 min) (100 mg, 23.30%) as a white solid. LCMS Rt=0.865 min, m/z=899.5 [M+H]⁺.

tert-butyl 3-((((6R,7R)-7-(6-(bis(4-methoxybenzyl)amino)-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidine-1-carboxylate (Peak 2, retention time=2.958 min) (100 mg, 23.30%) as a white solid. LCMS Rt=0.865 min, m/z=899.5 [M+H]⁺.

SFC (column: REGIS(S,S)WHELK-O1(250 mm*25 mm,10 um); mobile phase: [0.1% NH₃H₂O IPA]; B %: 60%-60%, 40 min).

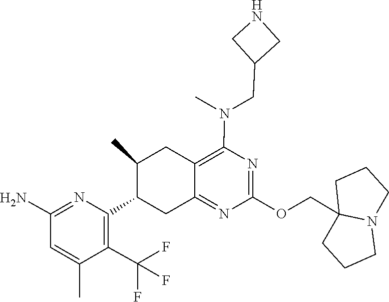 [SMILES: Cc1cc(N)nc([C@H]2Cc3nc(OCC45CCCN4CCC5)nc(N(C)CC4CNC4)c3C[C@@H]2C)c1C(F)(F)F]

### Step 3: (6S,7S)-7-(6-amino-4-methyl-3-(trifluoromethyl)pyridin-2-yl)-N-(azetidin-3-ylmethyl)-2-((hexahydro-1H-pyrrolizin-7a-yl)methoxy)-N,6-dimethyl-5,6,7,8-tetrahydroquinazolin-4-amine

The deprotection of Boc and PMB was prepared in a similar fashion to Example #65, Step 15. The reaction mixture was concentrated in vacuo and purified by reverse phase HPLC (column: Phenomenex Luna 80*30 mm*3 um; mobile phase: [water (trifluroacetic acid)-acetoniitrile]; B %: 5%-45%, 8 min) affording (6S,7S)-7-(6-amino-4-methyl-3-(trifluoromethyl)pyridin-2-yl)-N-(azetidin-3-ylmethyl)-2-((hexahydro-1H-pyrrolizin-7a-yl)methoxy)-N,6-dimethyl-5,6,7,8-tetrahydroquinazolin-4-amine (70 mg, 93.52%, trifluoroacetate salt) as a white solid: LCMS Rt=0.523 min, m/z=559.3 [M+H]⁺.

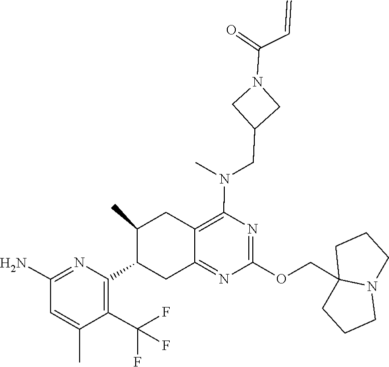 [SMILES: C=CC(=O)N1CC(CN(C)c2nc(OCC34CCCN3CCC4)nc3c2C[C@H](C)[C@@H](c2nc(N)cc(C)c2C(F)(F)F)C3)C1]

### Step 4: 1-(3-((((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

The amide coupling reaction was prepared in a similar fashion to Example #65, Step 16. The reaction mixture was concentrated in vacuo and purified by reverse phase HPLC (column: Waters Xbridge BEH C18 100*30 mm*10 um; mobile phase: [water (NH4HCO3)-acetonitrile]; B %: 30%-60%, 8 min) affording 1-(3-((((6S,7S)-7-(6-amino-4-methyl-3-(trifluoromethyl)pyridin-2-yl)-2-((hexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one (23.35 mg, 39.57%) as a yellow solid: ¹H NMR (400 MHz, Acetonitrile-d3) δ 6.35-6.22 (m, 2H), 6.19-6.12 (m, 1H), 5.66-5.59 (m, 1H), 5.22 (s, 2H), 4.28 (q, J=8.8 Hz, 1H), 4.09-3.83 (m, 5H), 3.79-3.57 (m, 1H), 3.51-3.37 (m, 1H), 3.17 (dt, J=6.3, 10.4 Hz, 1H), 3.02 (s, 3H), 3.01-2.92 (m, 3H), 2.87-2.77 (m, 1H), 2.70-2.55 (m, 3H), 2.53-2.43 (m, 1H), 2.38 (q, J=3.5 Hz, 3H), 2.17-2.03 (m, 2H), 1.95-1.86 (m, 2H), 1.85-1.69 (m, 4H), 1.63-1.53 (m, 2H), 0.76 (d, J=6.4 Hz, 3H). LCMS Rt=2.709 min, m/z=613.3 [M+H]⁺.

LCMS (5 to 95% acetonitrile in water+0.03% ammonium bicarbonate over 6 mins) retention time 2.709 min, ESI+ found [M+H]=613.3.

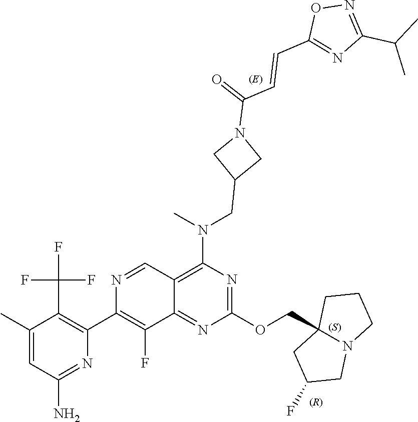

### Example 51: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

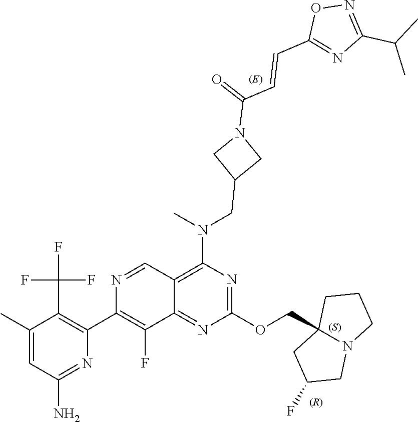

### Step 1: (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one

The amide coupling reaction was prepared in a similar fashion to Example #71, Step 11. The crude was purified by reverse phase HPLC (column: C18 (250*50 mm*10 um); mobile phase: [water (NH4HCO3)-acetonitrile]; B %: 40%-70%, 10 min) affording (E)-1-(3-(((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)-3-(3-isopropyl-1,2,4-oxadiazol-5-yl)prop-2-en-1-one (73.11 mg, 22.19%) as a pale yellow solid: ¹H NMR (400 MHz, Dimethyl sulfoxide-d6) δ 9.22 (s, 1H), 7.32-7.24 (m, 1H), 7.22-7.15 (m, 1H), 6.82-6.76 (m, 2H), 6.51-6.48 (m, 1H), 5.34-5.17 (m, 1H), 4.50 (t, J=8.6 Hz, 1H), 4.31 (br dd, J=5.6, 8.8 Hz, 1H), 4.15-4.10 (m, 2H), 4.09-3.98 (m, 2H), 3.90 (br dd, J=5.4, 10.4 Hz, 1H), 3.57 (s, 3H), 3.22-3.18 (m, 1H), 3.17-3.11 (m, 1H), 3.10-3.06 (m, 2H), 3.01 (br d, J=5.0 Hz, 1H), 2.84-2.78 (m, 1H), 2.38-2.35 (m, 3H), 2.17-2.02 (m, 2H), 2.00-1.95 (m, 1H), 1.86-1.69 (m, 4H), 1.30-1.27 (m, 6H). LCMS Rt=2.972 min, m/z=742.3 [M+H]⁺.

LCMS (5 to 95% acetonitrile in water+0.1% ammonium bicarbonate over 6 mins) retention time 2.972 min, ESI+ found [M+H]=742.3.

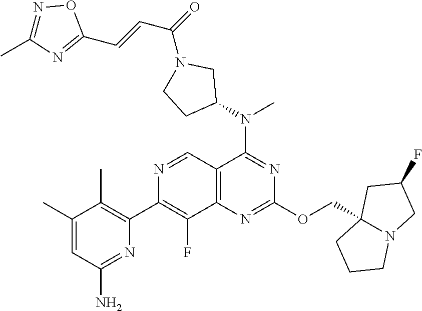 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1]

### Example 52: (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

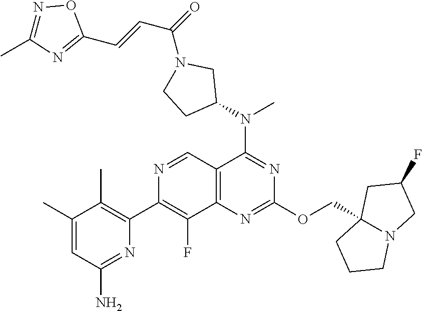 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1]

### Step 1: (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 30%-60%, 10 min) affording (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one (16.38 mg, 23.65%) as a yellow solid: ¹H NMR (400 MHz, Acetonitrile-d3) δ 9.23-9.14 (m, 1H), 7.51-7.31 (m, 2H), 6.54-6.43 (m, 1H), 5.44-5.14 (m, 2H), 4.77-4.64 (m, 2H), 4.23-4.17 (m, 1H), 4.13 (br d, J=2.8 Hz, 1H), 4.04-3.96 (m, 1H), 3.91-3.82 (m, 1H), 3.80-3.70 (m, 1H), 3.64-3.45 (m, 1H), 3.40 (s, 3H), 3.18-3.08 (m, 2H), 3.07-3.03 (m, 1H), 2.93-2.83 (m, 1H), 2.44-2.40 (m, 1H), 2.38 (d, J=8.1 Hz, 3H), 2.35-2.28 (m, 1H), 2.25 (s, 3H), 2.18 (br d, J=4.4 Hz, 2H), 2.12-1.99 (m, 4H), 1.90-1.76 (m, 3H). LCMS Rt=2.626 min, m/z=660.3 [M+H]⁺.

LCMS (5 to 95% acetonitrile in water+0.1% ammonium bicarbonate over 6 mins) retention time 2.626 min, ESI+ found [M+H]=660.3.

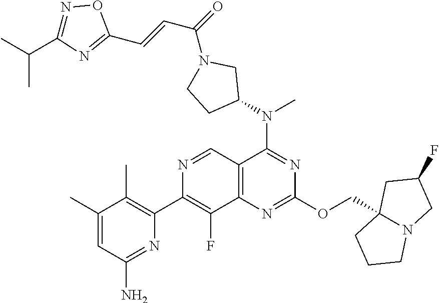 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C]

### Example 53: (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

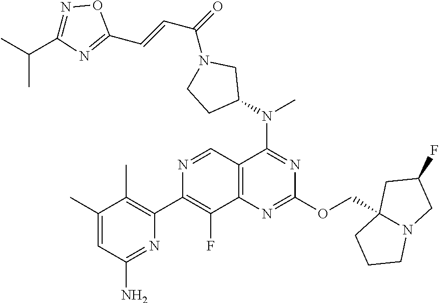 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C]

### Step 1: (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase HPLC(column: Waters Xbridge BEH C18 100*30 mm*10 um; mobile phase: [water (NH4HCO3)-acetonitrile]; B %: 40%-65%, 10 min) affording (E)-1-((R)-3-((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (23.28 mg, 32.24%) as a yellow solid: ¹H NMR (400 MHz, Acetonitrile-d3) δ 9.23-9.13 (m, 1H), 7.39 (d, J=11.5 Hz, 2H), 6.48 (s, 1H), 5.44-5.15 (m, 2H), 4.70 (s, 2H), 4.24-4.18 (m, 1H), 4.17-4.10 (m, 1H), 4.05-3.95 (m, 1H), 3.91-3.82 (m, 1H), 3.81-3.70 (m, 1H), 3.64-3.46 (m, 1H), 3.41 (s, 3H), 3.20-3.08 (m, 3H), 3.06 (br d, J=5.9 Hz, 1H), 2.88 (br s, 1H), 2.45-2.37 (m, 1H), 2.35-2.28 (m, 1H), 2.25 (s, 3H), 2.18 (br s, 2H), 2.10 (br d, J=2.4 Hz, 4H), 1.90-1.76 (m, 3H), 1.37-1.26 (m, 6H). LCMS Rt=2.843 min, m/z=688.3 [M+H]⁺.

LCMS (5 to 95% acetonitrile in water+0.1% ammonium bicarbonate over 6 mins) retention time 2.843 min, ESI+ found [M+H]=688.3.

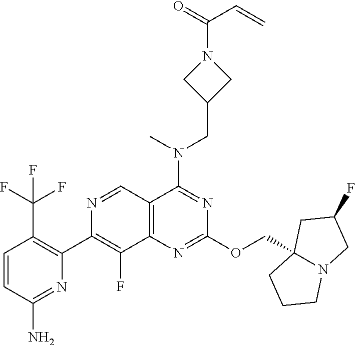 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1]

### Example 54: 1-(3-(((7-(6-amino-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one

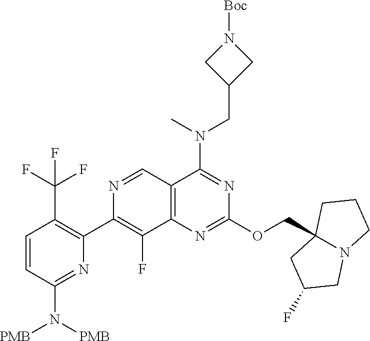

### Step 1: tert-butyl 3-(((7-(6-(bis(4-methoxybenzyl)amino)-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate

The Stille reaction was prepared in a similar fashion to Example #71, Step 6. The crude product was purified by column chromatography (silica gel, 100-200 mesh, 80-100% ethyl acetate in petroleum ether) affording tert-butyl 3-(((7-(6-(bis(4-methoxybenzyl)amino)-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidine-1-carboxylate (200 mg, 43.85%) as a yellow oil. LCMS Rt=0.811 min, m/z=904.4 [M+H]⁺.

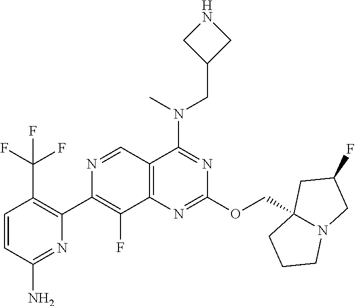 [SMILES: CN(CC1CNC1)c1nc(OC[C@@]23CCCN2C[C@H](F)C3)nc2c(F)c(-c3nc(N)ccc3C(F)(F)F)ncc12]

### Step 2: 7-(6-amino-3-(trifluoromethyl)pyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine

The deprotection of Boc and PMB group was prepared in a similar fashion to Example #71, Step 7. The mixture was purified by reverse phase HPLC(column: Phenomenex Luna 80*30 mm*3 um; mobile phase: [water (trifluroacetic acid)-acetoniitrile]; B %: 35%-75%, 8 min) affording 7-(6-amino-3-(trifluoromethyl)pyridin-2-yl)-N-(azetidin-3-ylmethyl)-8-fluoro-2-(((2R,7aS)-2-fluorohexahydro-1H-pyrrolizin-7a-yl)methoxy)-N-methylpyrido[4,3-d]pyrimidin-4-amine (60 mg, 44.45%, trifluoroacetate salt) as a white solid. LCMS Rt=0.547 min, m/z=564.2 [M+H]⁺.

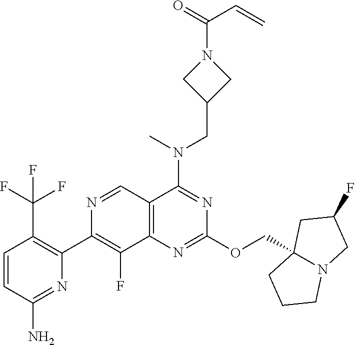 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1]

### Step 3: 1-(3-(((7-(6-amino-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 35%-65%, 8 min) affording 1-(3-(((7-(6-amino-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one (3.13 mg, 6.87%) as a white solid: ¹H NMR (400 MHz, Acetonitrile-d3) δ 9.22 (s, 1H), 7.84 (d, J=8.8 Hz, 1H), 6.73 (d, J=8.8 Hz, 1H), 6.37-6.25 (m, 1H), 6.22-6.13 (m, 1H), 5.69-5.55 (m, 3H), 5.39-5.18 (m, 1H), 4.35 (br t, J=8.4 Hz, 1H), 4.24-4.08 (m, 6H), 3.86 (br dd, J=5.6, 9.8 Hz, 1H), 3.56 (s, 3H), 3.22-3.07 (m, 4H), 2.97-2.88 (m, 1H), 2.12 (br s, 2H), 2.06 (br d, J=6.8 Hz, 1H), 1.92-1.77 (m, 3H). LCMS Rt=2.580 min, m/z=618.3 [M+H]⁺.

LCMS (5 to 95% acetonitrile in water+0.03% ammonium bicarbonate over 6 mins) retention time 2.580 min, ESI+ found [M+H]=618.3.

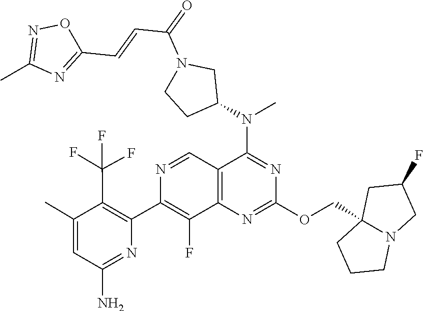 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

### Example 55: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

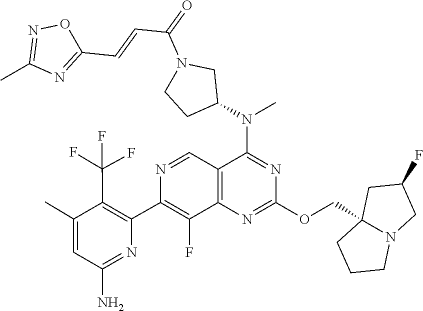 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1]

### Step 1: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #71, Step 11. the residue was purified by reverse phase HPLC (column: Waters Xbridge BEH C18 100*30 mm*10 um; mobile phase: [water (NH4HCO3)-acetonitrile]; B %: 25%-55%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one (18.07 mg, 28.86%) as a yellow solid: ¹H NMR (400 MHz, Acetonitrile-d3) δ 9.16 (d, J=1.9 Hz, 1H), 7.57-7.32 (m, 2H), 6.59 (s, 1H), 5.50-5.41 (m, 2H), 5.35-5.18 (m, 1H), 4.26-4.12 (m, 3H), 4.07-3.98 (m, 1H), 3.83-3.72 (m, 1H), 3.67-3.48 (m, 1H), 3.42 (s, 3H), 3.19-3.06 (m, 3H), 2.98-2.82 (m, 1H), 2.46 (d, J=1.6 Hz, 3H), 2.41 (d, J=8.4 Hz, 3H), 2.37-2.30 (m, 1H), 2.21 (br d, J=4.5 Hz, 2H), 2.14-2.04 (m, 3H), 1.94-1.82 (m, 3H). LCMS Rt=2.631 min, m/z=714.3 [M+H]⁺.

LCMS (5 to 95% acetonitrile in water+0.1% ammonium bicarbonate over 6 mins) retention time 2.631 min, ESI+ found [M+H]=714.3.

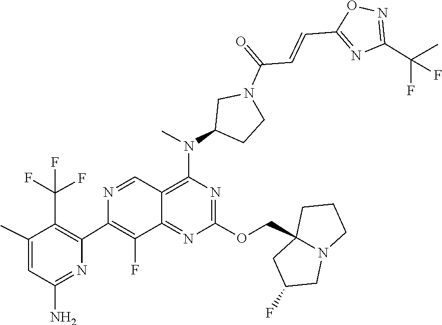 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F]

### Example 56: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one

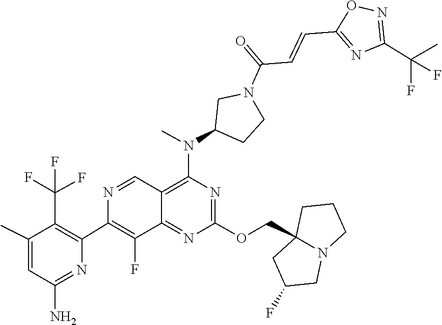 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F]

### Step 1: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one

The amide coupling reaction was prepared in a similar fashion to Example #71, Step 11. The crude was purified by reverse phase HPLC (column: Waters Xbridge Prep OBD C18 150*40 mm*10 um; mobile phase: [water (NH4HCO3)-acetonitrile]; B %: 30%-60%, 8 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-(1,1-difluoroethyl)-1,2,4-oxadiazol-5-yl)prop-2-en-1-one (16.38 mg, 23.65%) as a pale yellow amorphous solid: ¹H NMR (400 MHz, acetonitrile-d3) δ 9.06 (s, 1H), 7.62-7.24 (m, 2H), 6.49 (s, 1H), 5.43-5.28 (m, 2H), 5.27-5.09 (m, 1H), 4.19-4.02 (m, 3H), 3.93 (br dd, J=7.8, 13.1 Hz, 1H), 3.87-3.75 (m, 1H), 3.74-3.63 (m, 1H), 3.59-3.37 (m, 1H), 3.33 (s, 3H), 3.13-2.95 (m, 3H), 2.87-2.76 (m, 1H), 2.35-2.29 (m, 1H), 2.25 (br t, J=8.4 Hz, 1H), 2.18-2.05 (m, 5H), 2.04-1.96 (m, 4H), 1.84-1.72 (m, 3H). LCMS Rt=2.842 min, m/z=764.3 [M+H]⁺.

LCMS (5 to 95% acetonitrile in water+0.1% ammonium bicarbonate over 6 mins) retention time 2.842 min, ESI+ found [M+H]=764.3.

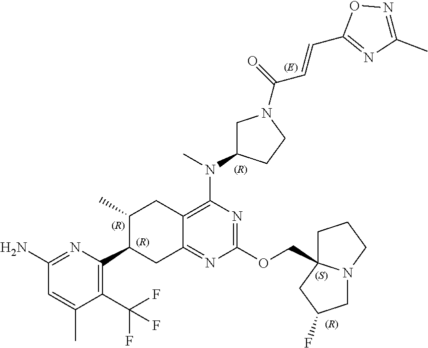

### Example 57: (E)-1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

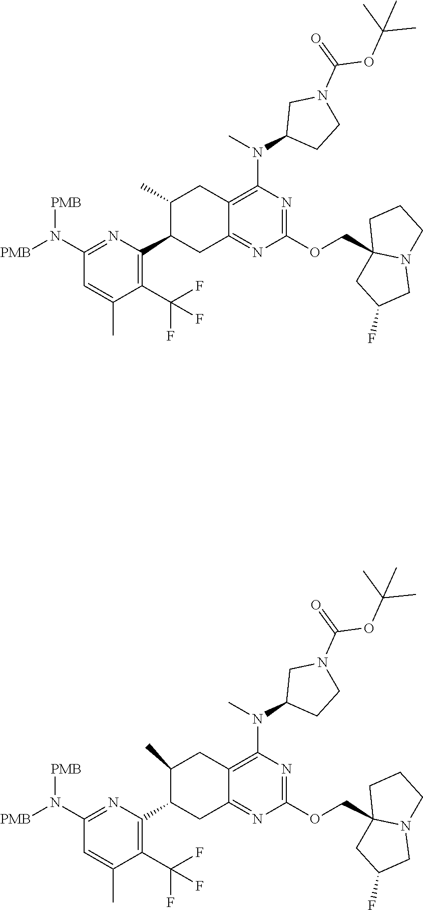

### Step 1: (R)-tert-butyl 3-(((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate and (R)-tert-butyl 3-(((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate

The mixture of diastereomers was prepared in the same manner as Example #65 and was further purified by SFC to give arbitrarily assigned:

(R)-tert-butyl 3-(((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate (Peak 1, retention time=1.969 min) (90 mg, 11.75%) as a yellow oil. LCMS Rt=0.842 min, m/z=917.5 [M+H]⁺.

(R)-tert-butyl 3-(((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate (Peak 2, retention time=2.182 min) (120 mg, 15.67%) as a yellow oil. LCMS Rt=0.842 min, m/z=917.5 [M+H]⁺.

SFC (column: (s,s) WHELK-01 (250 mm*30 mm,5 um); mobile phase: [0.1% ammonium hydroxide ethyl alcohol]; B %: 50%-50%, 16 min).

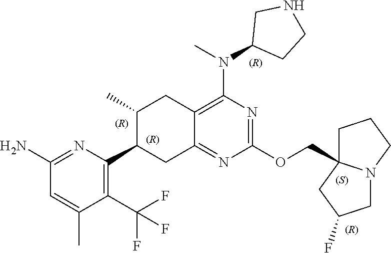

### Step 2: (6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-N,6-dimethyl-N—((R)-pyrrolidin-3-yl)-5,6,7,8-tetrahydroquinazolin-4-amine

The deprotection of Boc and PMB was prepared in a similar fashion to Example #65, Step 15. The crude product was purified by reverse phase HPLC (column: Phenomenex Luna 80*30 mm*3 um; mobile phase: [water(trifluoroacetic acid)-acetonitrile]; B %: 1%-40%, 8 min) affording (6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-N,6-dimethyl-N—((R)-pyrrolidin-3-yl)-5,6,7,8-tetrahydroquinazolin-4-amine (50 mg, 73.74%, trifluoroacetic salt) as a white solid. LCMS Rt=1.446 min, m/z=577.3 [M+H]⁺.

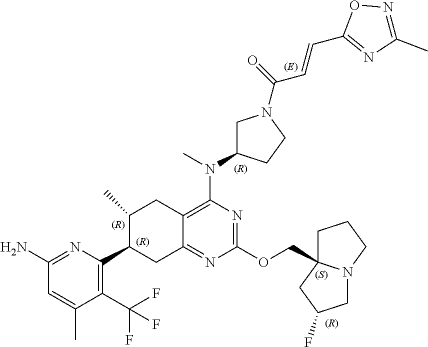

### Step 3: (E)-1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

The amide coupling reaction was prepared in a similar fashion to Example #65, Step 16. The crude product was purified by reverse phase HPLC (column: Waters Xbridge BEH C18 100*30 mm*10 um; mobile phase: [water(ammonium bicarbonate)-acetonitrile]; B %: 40%-70%, 10 min) affording (E)-1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (5.19 mg, 20.12%) as a yellow oil: ¹H NMR (400 MHz, Acetonitrile-d3) δ 7.51-7.31 (m, 2H), 6.28 (s, 1H), 5.36-5.15 (m, 3H), 4.79-4.59 (m, 1H), 4.19-4.03 (m, 2H), 4.02-3.91 (m, 2H), 3.86-3.75 (m, 1H), 3.73-3.62 (m, 1H), 3.51-3.39 (m, 1H), 3.17 (br t, J=8.2 Hz, 3H), 3.11-2.99 (m, 2H), 2.95-2.83 (m, 5H), 2.70-2.61 (m, 1H), 2.55-2.47 (m, 1H), 2.41 (s, 2H), 2.38 (br d, J=3.4 Hz, 3H), 2.26 (br s, 1H), 2.18 (br s, 1H), 2.13-2.03 (m, 4H), 1.93-1.77 (m, 3H), 0.77 (br d, J=6.3 Hz, 3H). LCMS Rt=2.968 min, m/z=713.3 [M+H]⁺.

LCMS (5 to 95% acetonitrile in water+0.03% ammonium bicarbonate over 6 mins) retention time 2.968 min, ESI+ found [M+H]=713.3.

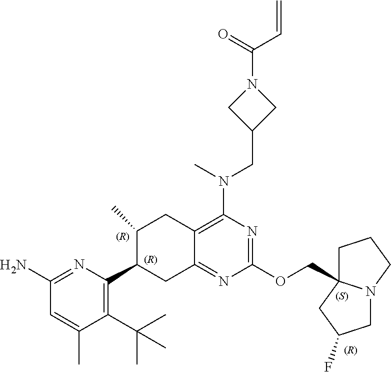

### Example 58: 1-(3-((((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

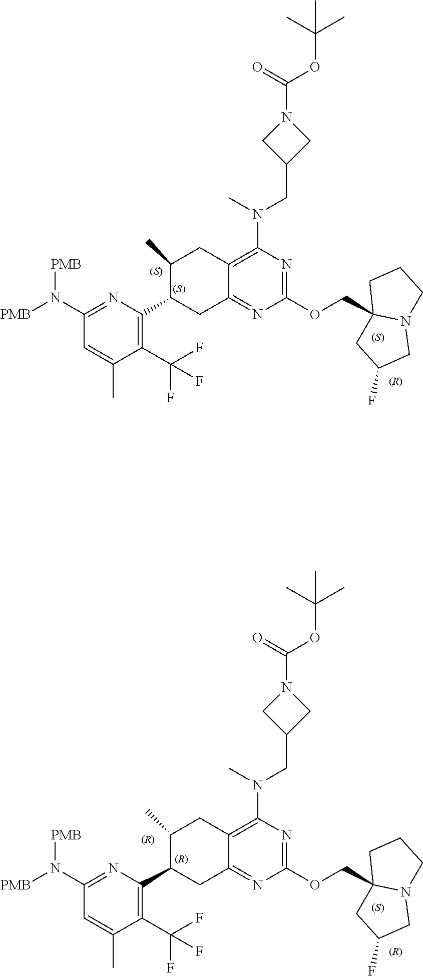

### Step 1: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate and tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate

The mixture of diastereomers was prepared in the same manner as Example #65 and was further separated by SFC to give arbitrarily assigned: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 1, retention time=1.687 min) (120 mg, 25.50%) as a white solid. LCMS Rt=0.670 min, m/z=917.5 [M+H]⁺. tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 2, retention time=2.228 min) (100 mg, 21.25%) as a white solid. LCMS Rt=0.670 min, m/z=917.5 [M+H]⁺. SFC (column: DAICEL CHIRALPAK IE(250 mm*30 mm,10 um); mobile phase: [acetonitrile/EtOH(0.1% NH₃H₂O)]; B %: 62%-62%, 35 min).

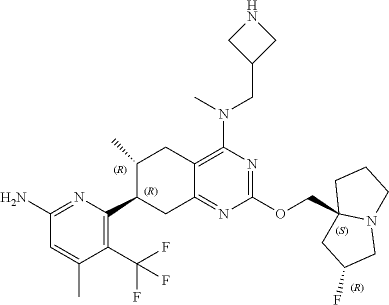

### Step 2: (6R,7R)-7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine

The deprotection of Boc and PMB was prepared in a similar fashion to Example #65, Step 15. The reaction mixture was concentrated in vacuo and purified by reverse phase HPLC (column: Phenomenex Luna 80*30 mm*3 um; mobile phase: [water (trifluroacetic acid)-acetoniitrile]; B %: 1%-30%, 8 min) affording (6R,7R)-7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine (70 mg, 92.91%, trifluoroacetate salt) as a white solid: LCMS Rt=0.434 min, m/z=577.3 [M+H]⁺.

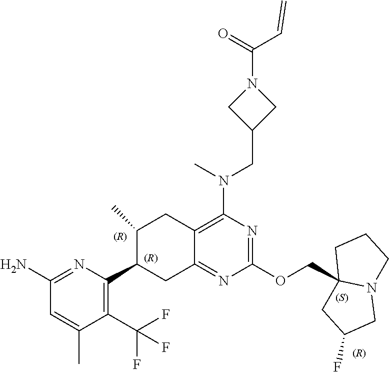

### Step 3: 1-(3-((((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

The amide coupling reaction was prepared in a similar fashion to Example #65, Step 16. The reaction mixture was concentrated in vacuo and purified by reverse phase HPLC (column: Waters Xbridge BEH C18 100*30 mm*10 um; mobile phase: [water (NH4HCO3)-acetonitrile]; B %: 35%-65%, 8 min) affording 1-(3-((((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one (20.65 mg, 32.30%) as a white solid: ¹H NMR (400 MHz, Acetonitrile-d3) δ 6.35-6.22 (m, 2H), 6.20-6.12 (m, 1H), 5.67-5.59 (m, 1H), 5.35-5.15 (m, 3H), 4.28 (q, J=8.5 Hz, 1H), 4.08-3.97 (m, 3H), 3.96-3.84 (m, 2H), 3.80-3.58 (m, 1H), 3.45 (ddd, J=7.0, 13.9, 17.2 Hz, 1H), 3.22-3.10 (m, 3H), 3.08-2.94 (m, 6H), 2.93-2.78 (m, 2H), 2.66 (dd, J=4.4, 15.8 Hz, 1H), 2.53-2.44 (m, 1H), 2.38 (q, J=3.3 Hz, 3H), 2.15-2.06 (m, 3H), 2.04-2.00 (m, 1H), 1.93-1.77 (m, 3H), 0.82-0.67 (m, 3H). LCMS Rt=2.855 min, m/z=631.3 [M+H]⁺.

LCMS (5 to 95% acetonitrile in water+0.03% ammonium bicarbonate over 6 mins) retention time 2.855 min, ESI+ found [M+H]=631.3.

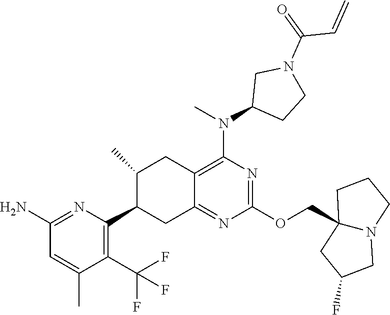 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1]

### Example 59:1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

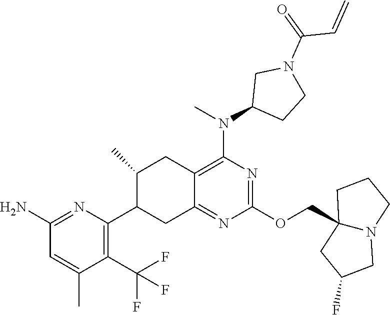 [SMILES: C=CC(=O)N1CC[C@@H](N(C)c2nc(OC[C@@]34CCCN3C[C@H](F)C4)nc3c2C[C@@H](C)C(c2nc(N)cc(C)c2C(F)(F)F)C3)C1]

### Step 1: 1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

The amide coupling reaction was prepared in a similar fashion to Example #65, Step 16. The crude product was purified by reverse phase HPLC (column: Waters Xbridge BEH C18 100*30 mm*10 um; mobile phase: [water (ammonium bicarbonate)-acetonitrile]; B %: 35%-60%, 10 min) affording 1-((R)-3-(((6R,7R)-7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one (5.54 mg, 26.37%) as a yellow oil: ¹H NMR (400 MHz, Acetonitrile-d3) δ 6.60 (dd, J=10.4, 16.8 Hz, 1H), 6.31-6.18 (m, 2H), 5.72-5.61 (m, 1H), 5.35-5.14 (m, 3H), 4.76-4.55 (m, 1H), 4.07-3.89 (m, 3H), 3.85-3.71 (m, 1H), 3.62-3.50 (m, 1H), 3.44-3.31 (m, 1H), 3.22-3.11 (m, 3H), 3.07 (br s, 1H), 3.03-2.97 (m, 1H), 2.95-2.80 (m, 5H), 2.68-2.60 (m, 1H), 2.55-2.45 (m, 1H), 2.38 (br d, J=3.5 Hz, 3H), 2.28 (br s, 1H), 2.15 (br s, 1H), 2.08 (br s, 2H), 2.04 (br dd, J=2.8, 9.6 Hz, 2H), 1.93-1.77 (m, 3H), 0.77 (d, J=6.4 Hz, 3H). LCMS Rt=2.864 min, m/z=631.3 [M+H]⁺.

LCMS (5 to 95% acetonitrile in water+0.03% ammonium bicarbonate over 6 mins) retention time 2.864 min, ESI+ found [M+H]=631.3.

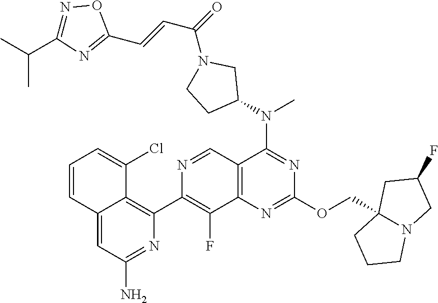 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1]

### Example 60: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

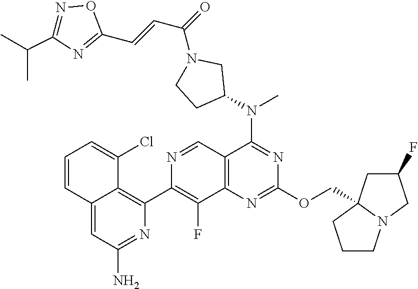 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1]

### Step 1: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was prepared in a similar fashion to Example #71, Step 11. The residue was purified by reverse phase reverse phase HPLC (column: Waters Xbridge Prep OBD C18 150*40 mm*10 um; mobile phase: [water (NH4HCO3)-acetonitrile]; B %: 25%-65%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (67.76 mg, 27.48%) as a yellow amorphous solid: ¹H NMR (400 MHz, Acetonitrile-d3) δ 9.18-9.13 (m, 1H), 7.64-7.60 (m, 1H), 7.47-7.31 (m, 3H), 7.24-7.19 (m, 1H), 6.90 (s, 1H), 5.45-5.24 (m, 2H), 5.18-5.13 (m, 2H), 4.27-4.06 (m, 3H), 3.98 (br d, J=7.8 Hz, 1H), 3.92-3.67 (m, 2H), 3.64-3.47 (m, 1H), 3.41 (s, 3H), 3.16-3.06 (m, 3H), 2.92-2.82 (m, 1H), 2.44-2.20 (m, 3H), 2.04 (br d, J=4.3 Hz, 2H), 1.88-1.72 (m, 3H), 1.31 (dd, J=7.0, 8.4 Hz, 6H). LCMS Rt=2.805 min, m/z=744.3 [M+H]⁺.

LCMS (5 to 95% acetonitrile in water+0.03% ammonium bicarbonate over 6 mins) retention time 2.805 min, ESI+ found [M+H]=744.3

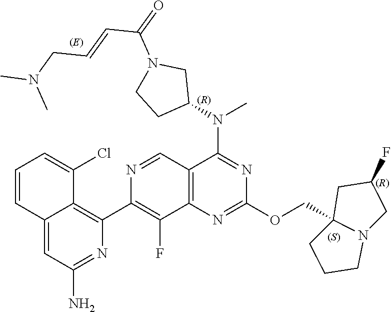

### Example 61: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one

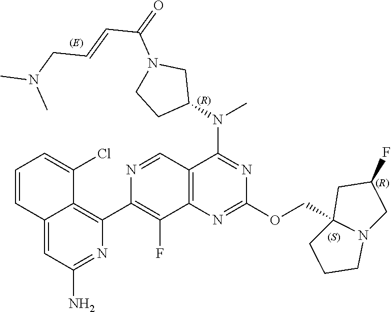

### Step 1: (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one

The amide coupling reaction was prepared in a similar fashion to Example #71, Step 11. The crude was purified by reverse phase HPLC (column: Waters Xbridge Prep OBD C18 150*40 mm*10 um; mobile phase: [water (NH4HCO3)-acetonitrile]; B %: 20%-50%, 8 min) affording (E)-1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one (10.1 mg, 8.45%) as a yellow amorphous solid: ¹H NMR (400 MHz, Acetonitrile-d3) δ 9.17 (d, J=3.4 Hz, 1H), 7.63 (d, J=8.4 Hz, 1H), 7.41 (t, J=7.9 Hz, 1H), 7.23 (d, J=7.3 Hz, 1H), 6.92 (s, 1H), 6.78-6.69 (m, 1H), 6.44-6.31 (m, 1H), 5.41-5.27 (m, 2H), 5.20-5.13 (m, 2H), 4.25-4.10 (m, 3H), 4.05 (br s, 1H), 3.97-3.76 (m, 3H), 3.68-3.49 (m, 3H), 3.40 (br s, 3H), 3.15-3.03 (m, 6H), 2.93-2.83 (m, 2H), 2.10 (br s, 5H), 1.92-1.82 (m, 3H). LCMS Rt=2.460 min, m/z=691.3 [M+H]⁺.

LCMS (5 to 95% acetonitrile in water+0.03% ammonium bicarbonate over 6 mins) retention time 2.460 min, ESI+ found [M+H]=691.3

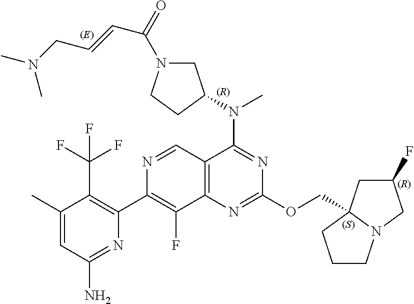

### Example 62: (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one

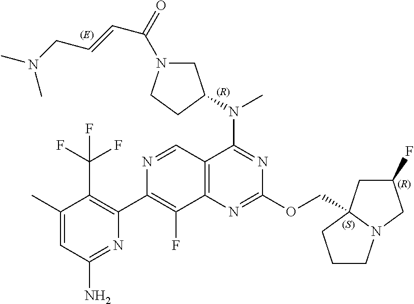

### Step 1: (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one

The amide coupling reaction was prepared in a similar fashion to Example #71, Step 11. The residue was purified by reverse phase HPLC (column: Waters Xbridge BEH C18 100*30 mm*10 um; mobile phase: [water (NH4HCO3)-acetonitrile]; B %: 25%-55%, 8 min) affording (E)-1-((R)-3-((7-(6-amino-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)-4-(dimethylamino)but-2-en-1-one (13.06 mg, 26.23%) as a yellow oil: ¹H NMR (400 MHz, Acetonitrile-d3) δ 9.15 (d, J=2.8 Hz, 1H), 6.81-6.68 (m, 1H), 6.59 (s, 1H), 6.45-6.33 (m, 1H), 5.48-5.35 (m, 2H), 5.34-5.20 (m, 1H), 4.27-4.19 (m, 1H), 4.18-4.11 (m, 1H), 4.05-3.84 (m, 1H), 3.69-3.43 (m, 2H), 3.41 (d, J=2.8 Hz, 3H), 3.21-3.12 (m, 2H), 3.11-3.04 (m, 3H), 2.96-2.87 (m, 1H), 2.46 (d, J=1.3 Hz, 3H), 2.42-2.27 (m, 2H), 2.22 (d, J=6.5 Hz, 6H), 2.17-2.05 (m, 5H), 1.92-1.84 (m, 3H). LCMS Rt=2.218 min, m/z=689.3 [M+H]⁺.

LCMS (5 to 95% acetonitrile in water+0.03% ammonium bicarbonate over 6 min) retention time 2.218 min, ESI+ found [M+H]=689.3.

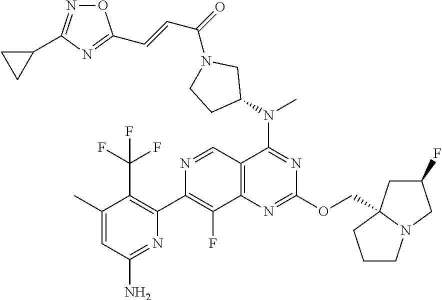 [SMILES: Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F]

### Example 63: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-cyclopropyl-1,2,4-oxadiazol-5-yl)prop-2-en-1-one

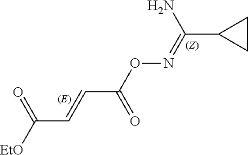 [SMILES: CCOC(=O)/C=C/C(=O)O/N=C(\N)C1CC1]

### Step 1: (E)-ethyl 4-(((Z)-(amino(cyclopropyl)methylene)amino)oxy)-4-oxobut-2-enoate

The amide coupling reaction was prepared in a similar fashion to Example #71, Step 8. The reaction mixture was concentrated in vacuo affording (E)-ethyl 4-(((Z)-(amino(cyclopropyl)methylene)amino)oxy)-4-oxobut-2-enoate (9 g, crude) as a brown oil, used in next step without further purification. LCMS Rt=0.539 min, m/z=226.1 [M+H]⁺.

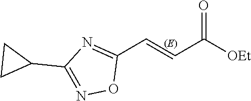 [SMILES: CCOC(=O)/C=C/c1nc(C2CC2)no1]

### Step 2: (E)-ethyl 3-(3-cyclopropyl-1,2,4-oxadiazol-5-yl)acrylate

The cyclization reaction was prepared in a similar fashion to Example #71, Step 9. The resulting residue was purified by column chromatography (silica gel, 100-200 mesh, 20% ethyl acetate in petroleum ether) affording (E)-ethyl 3-(3-cyclopropyl-1,2,4-oxadiazol-5-yl)acrylate (5.6 g, 67.61%) as a yellow oil. LCMS Rt=0.709 min, m/z=208.1 [M+H]⁺.

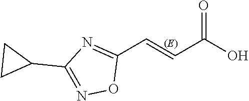 [SMILES: O=C(O)/C=C/c1nc(C2CC2)no1]

### Step 3: (E)-3-(3-cyclopropyl-1,2,4-oxadiazol-5-yl)acrylic acid

The hydrolysis reaction was prepared in a similar fashion to Example #71, Step 10.

The reaction mixture was concentrated in vacuo affording (E)-3-(3-cyclopropyl-1,2,4-oxadiazol-5-yl)prop-2-enoic acid (1.5 g, crude) as a white solid, used in next step without further purification: ¹H NMR (400 MHz, Chloroform-d) δ 7.51 (d, J=16.1 Hz, 1H), 6.98 (d, J=16.1 Hz, 1H), 2.19-2.14 (m, 1H), 1.14-1.04 (m, 4H).

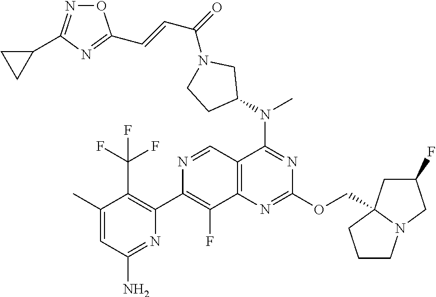 [SMILES: Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F]

### Step 4: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-cyclopropyl-1,2,4-oxadiazol-5-yl)prop-2-en-1-one

The amide coupling reaction was prepared in a similar fashion to Example #71, Step 11. The crude was purified by reverse phase HPLC (column: Waters Xbridge BEH C18 100*30 mm*10 um; mobile phase: [water (NH4HCO3)-acetonitrile]; B %: 32%-62%, 10 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-cyclopropyl-1,2,4-oxadiazol-5-yl)prop-2-en-1-one (13.2 mg, 24%) as a white solid: ¹H NMR (400 MHz, Acetonitrile-d3) δ 9.18-9.13 (m, 1H), 7.48-7.28 (m, 2H), 6.59 (s, 1H), 5.47 (br s, 2H), 5.34-5.16 (m, 1H), 4.26-4.20 (m, 1H), 4.19-4.13 (m, 1H), 4.05-3.96 (m, 1H), 3.81-3.72 (m, 1H), 3.65-3.51 (m, 1H), 3.42 (s, 3H), 3.20-3.07 (m, 3H), 2.95-2.87 (m, 1H), 2.46 (br s, 3H), 2.35-2.31 (m, 1H), 2.21 (br s, 2H), 2.13-2.07 (m, 4H), 1.93-1.83 (m, 4H), 1.14-1.09 (m, 2H), 1.03-0.97 (m, 2H). LCMS Rt=2.770 min, m/z=740.3 [M+H]⁺.

LCMS (5 to 95% acetonitrile in water+0.1% ammonium bicarbonate over 6 mins) retention time 2.770 min, ESI+ found [M+H]=740.3.

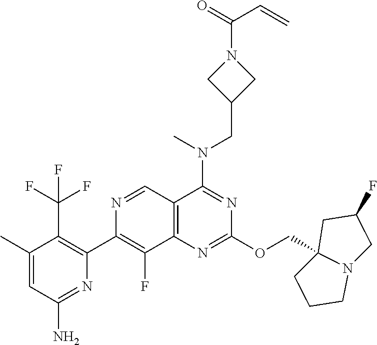 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1]

### Example 64: 1-(3-(((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

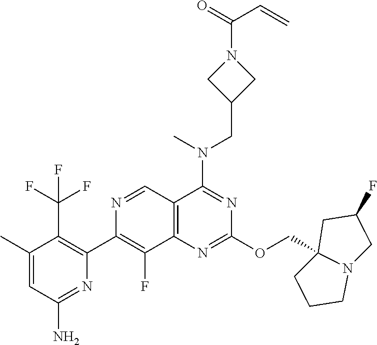 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1]

### Step 1: 1-(3-(((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

The amide coupling reaction was prepared in a similar fashion to Example #71, Step 11. The residue was purified by reverse phase HPLC (column: Waters Xbridge BEH C18 100*30 mm*10 um; mobile phase: [water (NH4HCO3)-acetonitrile]; B %: 25%-55%, 8 min) affording 1-(3-(((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one (5.35 mg, 12%) as a white solid: ¹H NMR (400 MHz, Acetonitrile-d3) δ 9.19 (s, 1H), 6.59 (s, 1H), 6.35-6.24 (m, 1H), 6.22-6.12 (m, 1H), 5.65 (dd, J=1.7, 10.2 Hz, 1H), 5.47 (br s, 2H), 5.38-5.18 (m, 1H), 4.35 (br t, J=8.4 Hz, 1H), 4.20 (br s, 1H), 4.15-4.00 (m, 4H), 3.86 (br dd, J=5.6, 9.8 Hz, 1H), 3.56 (s, 3H), 3.20-3.13 (m, 3H), 3.08 (br s, 1H), 2.95-2.87 (m, 1H), 2.46 (br s, 3H), 2.31-2.20 (m, 1H), 2.11 (br d, J=1.6 Hz, 1H), 2.09-2.04 (m, 1H), 1.94-1.78 (m, 4H). LCMS Rt=2.476 min, m/z=632.3 [M+H]⁺.

LCMS (5 to 95% acetonitrile in water+0.03% ammonium bicarbonate over 6 min) retention time 2.476 min, ESI+ found [M+H]=632.3

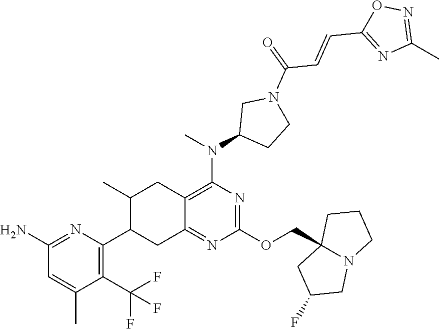 [SMILES: Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1]

### Example 65: (E)-1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

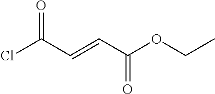

### Step 1: (E)-ethyl 4-chloro-4-oxobut-2-enoate

A mixture of (E)-4-ethoxy-4-oxo-but-2-enoic acid (13 g, 90.20 mmol), oxalyl dichloride (12.59 g, 99.22 mmol) and N,N-dimethylformaldehyde (659.27 mg, 9.02 mmol) in dichloromethane (80 mL) was degassed and purged with nitrogen for 3 times, and then the mixture was stirred at 20° C. for 0.5 h. The reaction mixture was concentrated in vacuo affording (E)-ethyl 4-chloro-4-oxobut-2-enoate (14 g, crude) as a yellow oil used in the next step without further purification.

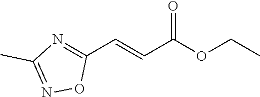

### Step 2: (E)-ethyl 3-(3-methyl-1,2,4-oxadiazol-5-yl)acrylate

The cyclization reaction was prepared in a similar fashion to Example #71, Step 9. The crude product was concentrated in vacuo and purified by column chromatography (silica gel, 100-200 mesh, 0-10% ethyl acetate in petroleum ether) affording (E)-ethyl 3-(3-methyl-1,2,4-oxadiazol-5-yl)acrylate (4.9 g, 85%) as a brown oil: ¹H NMR (400 MHz, Chloroform-d) δ 7.46-7.34, (m, 1H), 6.98-6.88 (m, 1H), 4.24 (q, J=7.13 Hz, 2H), 2.38 (s, 3H), 1.32-1.26 (m, 3H). LCMS Rt=0.383 min, m/z=182.1 [M+H]⁺.

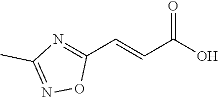

### Step 3: (E)-3-(3-methyl-1,2,4-oxadiazol-5-yl)acrylic acid

The hydrolysis reaction was prepared in a similar fashion to Example #71, Step 10. The crude product was concentrated in vacuo affording (E)-3-(3-methyl-1,2,4-oxadiazol-5-yl)acrylic acid (860 mg, crude) as a colorless oil used in the next step without further purification: ¹H NMR (400 MHz, Methanol-d4) δ 7.34 (d, J=16.1 Hz, 1H), 6.87 (d, J=16.1 Hz, 1H), 2.31 (s, 3H). LCMS Rt=0.200 min, m/z=154.0 [M+H]⁺.

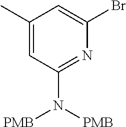

### Step 4: 6-bromo-N,N-bis(4-methoxybenzyl)-4-methylpyridin-2-amine

The PMB protection reaction was prepared in a similar fashion to Example #71, Step 1. The crude product was triturated with water (50 mL), filtered, the filter cake was collected and concentrated in vacuo affording 6-bromo-N,N-bis(4-methoxybenzyl)-4-methylpyridin-2-amine (220 g, 96.29%) as a yellow solid: ¹H NMR (400 MHz, Chloroform-d) δ 7.07 (d, J=8.6 Hz, 4H), 6.80-6.73 (m, 4H), 6.51 (s, 1H), 6.07 (s, 1H), 4.55 (s, 4H), 3.71 (s, 6H), 2.04 (s, 3H). LCMS Rt=2.438 min, m/z=426.1 [M+H]⁺.

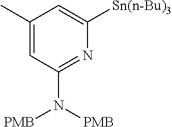

### Step 5: N,N-bis(4-methoxybenzyl)-4-methyl-6-(tributylstannyl)pyridin-2-amine

To a solution of 6-bromo-N,N-bis[(4-methoxyphenyl)methyl]-4-methyl-pyridin-2-amine (50 g, 117 mmol) in dioxane (4.0 L) was added lithium chloride (24.8 g, 582.02 mmol), tricyclohexylphosphane (6.56 g, 23.4 mmol), tricyclohexylphosphane (6.56 g, 23.4 mmol), (1E,4E)-1,5-diphenylpenta-1,4-dien-3-one;palladium (10.72 g, 11.7 mmol) and tributyl(tributylstannyl)stannane (169.68 g, 292.52 mmol). The mixture was stirred at 110° C. for 12 h under nitrogen atmosphere. The reaction mixture was filtered and the filtrate was concentrated to dryness in vacuo. The residue was purified by column chromatography (silica gel, 100-200 mesh, 0-10% ethyl acetate in petroleum ether) affording N,N-bis(4-methoxybenzyl)-4-methyl-6-(tributylstannyl)pyridin-2-amine (74 g, 99.21%) as a yellow oil. LCMS Rt=2.407 min, m/z=638.3 [M+H]⁺.

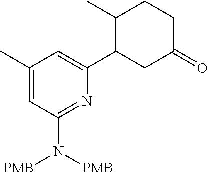

### Step 6: 3-(6-(bis(4-methoxybenzyl)amino)-4-methylpyridin-2-yl)-4-methylcyclohexanone

To a solution of N,N-bis[(4-methoxyphenyl)methyl]-4-methyl-6-tributylstannyl-pyridin-2-amine (38 g, 59.61 mmol) in tetrahydrofuran (2000 mL) was added chlororhodium;(1Z,5Z)-cycloocta-1,5-diene (2.94 g, 5.96 mmol, 0.1 eq), water (107.39 mg, 5.96 mmol) and 4-methylcyclohex-2-en-1-one (7.88 g, 71.53 mmol). The mixture was stirred at 60° C. for 12 h under nitrogen atmosphere. The reaction mixture was concentrated to dryness in vacuo. The residue was purified by column chromatography (silica gel, 100-200 mesh, 0-10% ethyl acetate in petroleum ether) affording 3-(6-(bis(4-methoxybenzyl)amino)-4-methylpyridin-2-yl)-4-methylcyclohexanone (35 g, 64.02%) as a yellow oil. LCMS Rt=1.824 min, m/z=458.3 [M+H]⁺.

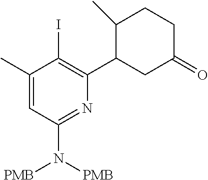

### Step 7: 3-(6-(bis(4-methoxybenzyl)amino)-3-iodo-4-methylpyridin-2-yl)-4-methylcyclohexanone

To a solution of 3-(6-(bis(4-methoxybenzyl)amino)-4-methylpyridin-2-yl)-4-methylcyclohexanone (34 g, 74.14 mmol) in N,N-dimethylformaldehyde (500 mL) was added N-iodo-succinimide (33.36 g, 148.28 mmol). The mixture was stirred at 25° C. for 3 h. The reaction mixture was concentrated to dryness in vacuo. The residue was purified by column chromatography (silica gel, 100-200 mesh, 0-10% ethyl acetate in petroleum ether) affording 3-(6-(bis(4-methoxybenzyl)amino)-3-iodo-4-methylpyridin-2-yl)-4-methylcyclohexanone (19 g, 43.85%) as a yellow oil. LCMS Rt=1.092 min, m/z=584.2 [M+H]⁺.

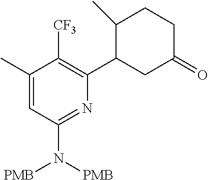

### Step 8: 3-(6-(bis(4-methoxybenzyl)amino)-4-methyl-3-(trifluoromethyl)pyridin-2-yl)-4-methylcyclohexanone

To a solution of 3-(6-(bis(4-methoxybenzyl)amino)-3-iodo-4-methylpyridin-2-yl)-4-methylcyclohexanone (15 g, 25.66 mmol) in N,N-dimethylformaldehyde (300 mL) was added cuprous iodide (14.66 g, 76.99 mmol) and methyl 2,2-difluoro-2-fluorosulfonyl-acetate (24.65 g, 128.32 mmol). The mixture was stirred at 90° C. for 2 h under nitrogen atmosphere. The reaction mixture was filtered and the filtrate was concentrated to dryness in vacuo. The residue was purified by column chromatography (silica gel, 100-200 mesh, 0-10% ethyl acetate in petroleum ether) affording 3-(6-(bis(4-methoxybenzyl)amino)-4-methyl-3-(trifluoromethyl)pyridin-2-yl)-4-methylcyclohexanone (12 g, 88.80%) as a yellow oil. LCMS Rt=0.875 min, m/z=526.2 [M+H]⁺.

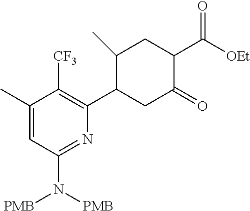

### Step 9: ethyl 4-(6-(bis(4-methoxybenzyl)amino)-4-methyl-3-(trifluoromethyl)pyridin-2-yl)-5-methyl-2-oxocyclohexanecarboxylate

To a solution of 3-(6-(bis(4-methoxybenzyl)amino)-4-methyl-3-(trifluoromethyl)pyridin-2-yl)-4-methylcyclohexanone (12 g, 22.78 mmol) in tetrahydrofuran (600 mL) was added lithium hexamethyldisilazane (1 M, 34.18 mL) at −78° C. under nitrogen atmosphere for 1 h, then ethyl cyanoformate (2.7 g, 27.34 mmol) was added at −78° C. The resulting mixture was stirred at −78° C. for 0.5 h under nitrogen atmosphere. The reaction mixture was quenched with a saturated solution of ammonium chloride (100 mL) and extracted with ethyl acetate (3×150 mL). The combined organic layers were dried over sodium sulphate and concentrated in vacuo affording ethyl 4-[6-[bis[(4-methoxyphenyl)methyl]amino]-4-methyl-3-(trifluoromethyl)-2-pyridyl]-5-methyl-2-oxo-cyclohexanecarboxylate (13.6 g, crude) as a yellow oil used in the next step without further purification. LCMS Rt=1.235 min, m/z=598.3 [M+H]⁺.

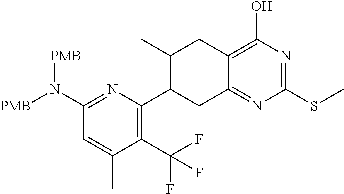

### Step 10: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol

To a solution of ethyl ethyl 4-(6-(bis(4-methoxybenzyl)amino)-4-methyl-3-(trifluoromethyl)pyridin-2-yl)-5-methyl-2-oxocyclohexanecarboxylate (13 g, 21.72 mmol) in ethanol (1200 mL)/water (240 mL) was added sodium bicarbonate (45.61 g, 542.89 mmol) and 2-methylisothiourea;sulfuric acid (60.45 g, 217.15 mmol). The mixture was stirred at 50° C. for 12 h. The reaction mixture was filtered and the filtrate was concentrated to dryness in vacuo. The residue was purified by column chromatography (silica gel, 100-200 mesh, 0-10% ethyl acetate in petroleum ether) affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (4.7 g, 34.65%) as a yellow solid. LCMS Rt=1.046 min, m/z=624.2 [M+H]⁺.

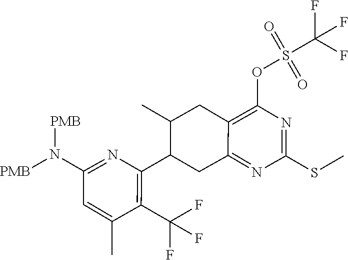

### Step 11: 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate

To a solution of 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-ol (1.5 g, 2.40 mmol) in dichloromethane (100 mL) was added triethylamine (970 mg, 9.60 mmol) and trifluoromethane anhydride (2.37 g, 8.40 mmol) at 0° C. under nitrogen atmosphere. The mixture was stirred at 25° C. for 1 h. The reaction mixture was quenched with a saturated solution of sodium bicarbonate (30 mL) at 0° C. and extracted with dichloromethane (3×100 mL). The combined organic layers were dried over sodium sulphate and concentrated in vacuo affording 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate (1.8 g, crude) as a yellow oil used in the next step without further purification. LCMS Rt=3.664 min, m/z=756.2 [M+H]⁺.

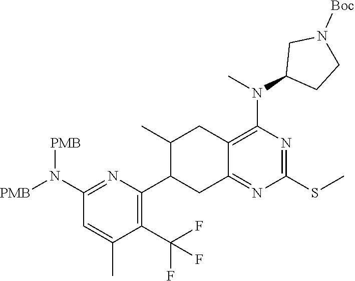

### Step 12: (3R)-tert-butyl 3-((7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)(methyl)amino)pyrrolidine-1-carboxylate

To a solution of tert-butyl (3R)-3-(methylamino)pyrrolidine-1-carboxylate (714.54 mg, 3.57 mmol) in N,N-dimethylformaldehyde (10 mL) was added N-ethyl-N-isopropylpropan-2-amine (1.23 g, 9.51 mmol) and 7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl trifluoromethanesulfonate (1.8 g, 2.38 mmol) at 0° C. The mixture was stirred at 20° C. for 12 h. The reaction mixture was concentrated in vacuo and purified by column chromatography (silica gel, 100-200 mesh, 0-50% ethyl acetate in petroleum ether) affording (3R)-tert-butyl 3-((7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)(methyl)amino)pyrrolidine-1-carboxylate (1.3 g, 67.73%) as a yellow oil. LCMS Rt=0.902 min, m/z=806.4 [M+H]⁺.

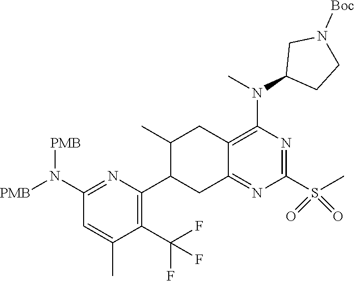

### Step 13: (3R)-tert-butyl 3-((7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)(methyl)amino)pyrrolidine-1-carboxylate

To a solution of (3R)-tert-butyl 3-((7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)(methyl)amino)pyrrolidine-1-carboxylate (1.3 g, 1.61 mmol) in dichloromethane (30 mL) was added metachloroperbenzoic acid (654.11 mg, 3.22 mmol, 85% purity) at 0° C. The mixture was stirred at 0° C. for 1 h. The reaction mixture was quenched with a saturated solution of sodium sulfite (15 mL) and extracted with dichloromethane (3×30 mL). The combined organic layers were dried over sodium sulphate and concentrated in vacuo affording (3R)-tert-butyl 3-((7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)(methyl)amino)pyrrolidine-1-carboxylate (1 g, crude) as a yellow oil used in the next step without further purification. LCMS Rt=1.128 min, m/z=838.4 [M+H]⁺.

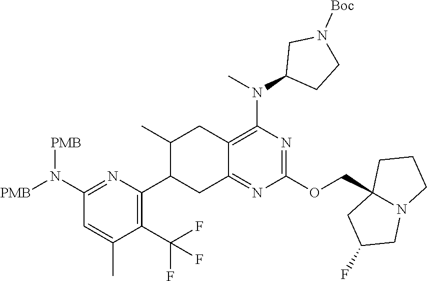

### Step 14: (3R)-tert-butyl 3-((7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate

To a solution of (3R)-tert-butyl 3-((7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)(methyl)amino)pyrrolidine-1-carboxylate (700 mg, 834.35 umol) in Toluene (30 mL) was added sodium tert-butoxide (2 M, 834.35 uL) and 4A MS (100 mg) and [(2R,8S)-2-fluoro-1,2,3,5,6,7-hexahydropyrrolizin-8-yl]methanol (199.24 mg, 1.25 mmol) at −30° C. under nitrogen atmosphere. The mixture was stirred at −30° C. for 10 min under nitrogen. The reaction mixture was quenched with a saturated solution of ammonium chloride (30 mL) and extracted with ethyl acetate (3×100 mL). The combined organic layers were dried over sodium sulphate and concentrated in vacuo. The residue was purified by column chromatography (silica gel, 100-200 mesh, 0-100% ethyl acetate in petroleum ether) affording (3R)-tert-butyl 3-((7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate (500 mg, 65.28%) as a yellow oil. LCMS Rt=0.842 min, m/z=917.5 [M+H]⁺.

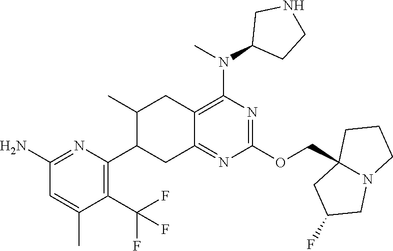 [SMILES: Cc1cc(N)nc(C2Cc3nc(OC[C@@]45CCCN4C[C@H](F)C5)nc(N(C)[C@@H]4CCNC4)c3CC2C)c1C(F)(F)F]

### Step 15: 7-(6-amino-4-methyl-3-(trifluoromethyl)pyridin-2-yl)-2-(((2R,7aS)-2-fluorohexahydro-1H-pyrrolizin-7a-yl)methoxy)-N,6-dimethyl-N—((R)-pyrrolidin-3-yl)-5,6,7,8-tetrahydroquinazolin-4-amine

A solution of (3R)-tert-butyl 3-((7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidine-1-carboxylate (150 mg, 163.39 umol) in trifluoroacetic acid (1 mL) was stirred at 80° C. for 0.5 h. The reaction mixture was concentrated in vacuo and purified by reverse phase HPLC (column: Phenomenex Luna 80*30 mm*3 um; mobile phase: [water (trifluroacetic acid)-acetoniitrile]; B %: 1%-40%, 8 min) affording 7-(6-amino-4-methyl-3-(trifluoromethyl)pyridin-2-yl)-2-(((2R,7aS)-2-fluorohexahydro-1H-pyrrolizin-7a-yl)methoxy)-N,6-dimethyl-N—((R)-pyrrolidin-3-yl)-5,6,7,8-tetrahydroquinazolin-4-amine (70 mg, 61.94%, trifluoroacetate salt) as a white solid. LCMS Rt=0.590 min, m/z=577.3 [M+H]⁺.

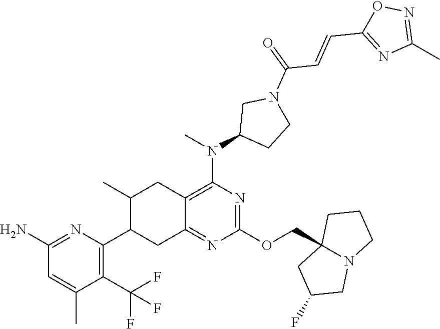 [SMILES: Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1]

### Step 16: (E)-1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

To a solution of 7-(6-amino-4-methyl-3-(trifluoromethyl)pyridin-2-yl)-2-(((2R,7aS)-2-fluorohexahydro-1H-pyrrolizin-7a-yl)methoxy)-N,6-dimethyl-N—((R)-pyrrolidin-3-yl)-5,6,7,8-tetrahydroquinazolin-4-amine (44 mg, 63.61 umol, trifluoroacetate salt) in dichloromethane (2 mL) was added (E)-3-(3-methyl-1,2,4-oxadiazol-5-yl)prop-2-enoic acid (7.84 mg, 50.89 umol), N-ethyl-N-isopropylpropan-2-amine (32.89 mg, 254.45 umol) and 2,4,6-tripropyl-1,3,5,2,4,6-trioxatriphosphinane 2,4,6-trioxide (80.96 mg, 127.23 umol, 50% purity). The mixture was stirred at −10° C. for 0.5 h. The mixture was concentrated to dryness in vacuo. The crude product was purified by reverse phase prep-HPLC (column: Waters Xbridge BEH C18 100*30 mm*10 um; mobile phase: [water (NH4HCO3)-acetonitrile]; B %: 40%-60%, 8 min) affording (E)-1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (7.67 mg, 16.89%) as a yellow oil: ¹H NMR (400 MHz, Acetonitrile-d3) δ 7.54-7.33 (m, 2H), 6.29 (s, 1H), 5.36-5.16 (m, 3H), 4.80-4.59 (m, 1H), 4.12-3.92 (m, 3H), 3.88-3.78 (m, 1H), 3.77-3.60 (m, 1H), 3.53-3.40 (m, 1H), 3.32 (s, 1H), 3.24-3.12 (m, 3H), 3.09-2.99 (m, 2H), 2.94 (br d, J=4.9 Hz, 3H), 2.89 (br d, J=5.1 Hz, 2H), 2.74-2.62 (m, 1H), 2.58-2.48 (m, 1H), 2.43 (s, 1H), 2.41 (d, J=2.4 Hz, 1H), 2.39 (d, J=3.4 Hz, 2H), 2.31 (br s, 2H), 2.18 (br d, J=4.8 Hz, 2H), 2.09 (br s, 2H), 2.06-2.01 (m, 1H), 1.94-1.76 (m, 3H), 0.79 (br d, J=6.2 Hz, 3H). LCMS Rt=2.971/3.009 min, m/z=713.3 [M+H]⁺.

LCMS (5 to 95% acetonitrile in water+0.03% ammonium bicarbonate over 6 mins) retention time 2.971/3.009 min, ESI+ found [M+H]=713.3.

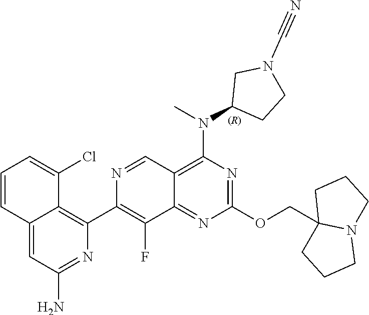

### Example 66: (R)-3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile

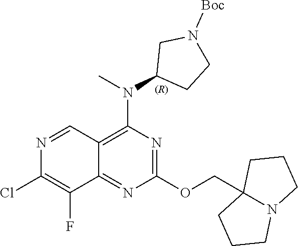

### Step 1: (R)-tert-butyl 3-((7-chloro-8-fluoro-2-((hexahydro-1H-pyrrolizin-7a-yl)methoxy)pyrido[4,3-d]pyrimidin-4-yl)(methyl)amino)pyrrolidine-1-carboxylate

The substitution reaction was prepared in a similar fashion to Example #71, Step 4. The mixture was concentrated in vacuo affording (R)-tert-butyl 3-((7-chloro-8-fluoro-2-((hexahydro-1H-pyrrolizin-7a-yl)methoxy)pyrido[4,3-d]pyrimidin-4-yl)(methyl)amino)pyrrolidine-1-carboxylate (12 g, crude) as a yellow oil, used in next step without any further purification. LCMS Rt=0.844 min, m/z=520.2 [M+H]⁺.

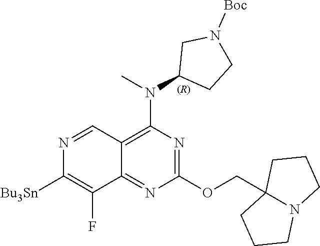

### Step 2: (R)-tert-butyl 3-((8-fluoro-2-((hexahydro-1H-pyrrolizin-7a-yl)methoxy)-7-(tributylstannyl)pyrido[4,3-d]pyrimidin-4-yl)(methyl)amino)pyrrolidine-1-carboxylate

The tin reagent formation was prepared in a similar fashion to Example #71, Step 5. The mixture was purified by column chromatography (silica gel, 100-200 mesh, 80-100% tetrahydrofuran in petroleum ether) affording (R)-tert-butyl 3-((8-fluoro-2-((hexahydro-1H-pyrrolizin-7a-yl)methoxy)-7-(tributylstannyl)pyrido[4,3-d]pyrimidin-4-yl)(methyl)amino)pyrrolidine-1-carboxylate (200 mg, 11.76%) as a yellow oil. LCMS Rt=0.851 min, m/z=776.4 [M+H]⁺.

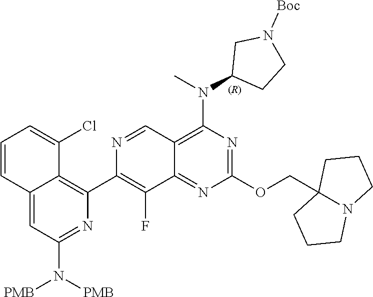

### Step 3: (R)-tert-butyl 3-((7-(3-(bis(4-methoxybenzyl)amino)-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6. The mixture was purified by reverse phase HPLC (column: Phenomenex Luna 80*30 mm*3 um; mobile phase: [water (trifluroacetic acid)-acetoniitrile]; B %: 45%-75%, 8 min) affording (R)-tert-butyl 3-((7-(3-(bis(4-methoxybenzyl)amino)-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carboxylate (40 mg, 16.31%, trifluoroacetate salt) as a yellow oil. LCMS Rt=2.754 min, m/z=902.4 [M+H]⁺.

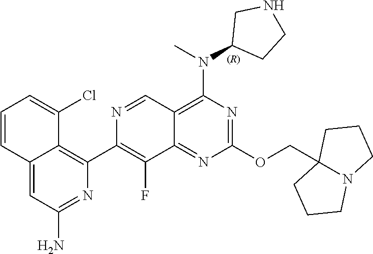

### Step 4: (R)-7-(3-amino-8-chloroisoquinolin-1-yl)-8-fluoro-2-((hexahydro-1H-pyrrolizin-7a-yl)methoxy)-N-methyl-N-(pyrrolidin-3-yl)pyrido[4,3-d]pyrimidin-4-amine

The deprotection of Boc and PMB was prepared in a similar fashion to Example #71, Step 7. The mixture was concentrated in vacuo affording (R)-7-(3-amino-8-chloroisoquinolin-1-yl)-8-fluoro-2-((hexahydro-1H-pyrrolizin-7a-yl)methoxy)-N-methyl-N-(pyrrolidin-3-yl)pyrido[4,3-d]pyrimidin-4-amine (40 mg, crude, trifluoroacetate salt) as a yellow oil, used in the next step without further purification. LCMS Rt=0.576 min, m/z=562.2 [M+H]⁺.

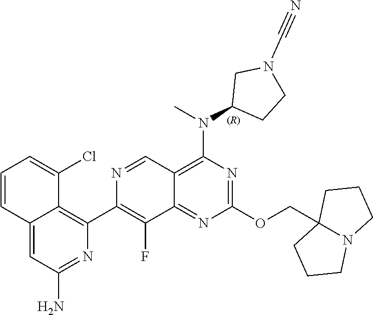

### Step 5: (R)-3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile

The substitution reaction was prepared in a similar fashion to Example #71, Step 6. The crude product was purified by reverse phase HPLC (column: Waters Xbridge BEH C18 100*30 mm*10 um; mobile phase: [water (NH4HCO3)-acetonitrile]; B %: 15%-45%, 8 min) affording (R)-3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile (1.10 mg, 4.00%) as a yellow solid: ¹H NMR (400 MHz, Acetonitrile-d3) δ 9.21-9.14 (m, 1H), 7.69-7.63 (m, 1H), 7.47-7.41 (m, 1H), 7.28-7.23 (m, 1H), 6.93 (s, 1H), 5.47-5.33 (m, 1H), 5.26-5.01 (m, 2H), 4.32-4.13 (m, 2H), 3.87-3.77 (m, 1H), 3.74-3.64 (m, 1H), 3.57-3.49 (m, 2H), 3.44 (d, J=0.9 Hz, 3H), 3.14-3.01 (m, 2H), 2.76-2.62 (m, 2H), 2.39 (ddt, J=4.3, 7.9, 12.4 Hz, 1H), 2.33-2.24 (m, 1H), 1.95-1.77 (m, 6H), 1.77-1.63 (m, 2H). LCMS Rt=1.980 min, m/z=587.2 [M+H]⁺.

LCMS (5 to 95% acetonitrile in water+0.1% trifluoroacetic acid over 6 mins) retention time 1.980 min, ESI+ found [M+H]=587.2.

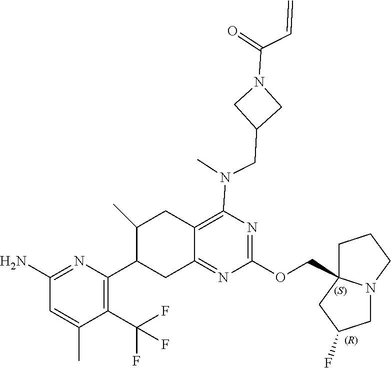

### Example 67: 1-(3-(((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

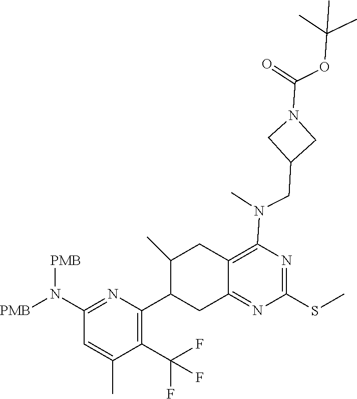

### Step 1: tert-butyl 3-(((7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)(methyl)amino)methyl)azetidine-1-carboxylate

The substitution reaction was prepared in a similar fashion to Example #65, Step 12. The resulting residue was purified by column chromatography (silica gel, 100-200 mesh, 0-100% ethyl acetate in petroleum ether) affording tert-butyl 3-(((7-(6-(bis(4-methoxybenzyl)amino)-4-methyl-3-(trifluoromethyl)pyridin-2-yl)-6-methyl-2-(methylthio)-5,6,7,8-tetrahydroquinazolin-4-yl)(methyl)amino)methyl)azetidine-1-carboxylate (1.7 g, 79.71%) as a yellow solid. LCMS Rt=0.958 min, m/z=806.4 [M+H]⁺.

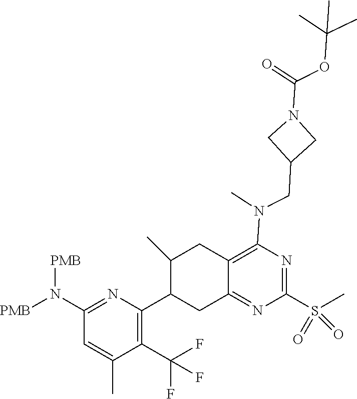

### Step 2: tert-butyl 3-(((7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)(methyl)amino)methyl)azetidine-1-carboxylate

The oxidation reaction was prepared in a similar fashion to Example #65, Step 13. The resulting residue was purified by column chromatography (silica gel, 100-200 mesh, 0-100% ethyl acetate in petroleum ether) affording tert-butyl 3-(((7-(6-(bis(4-methoxybenzyl)amino)-4-methyl-3-(trifluoromethyl)pyridin-2-yl)-6-methyl-2-(methylsulfonyl)-5,6,7,8-tetrahydroquinazolin-4-yl)(methyl)amino)methyl)azetidine-1-carboxylate (900 mg, 54.10%) as a yellow solid. LCMS Rt=0.867 min, m/z=838.4 [M+H]⁺.

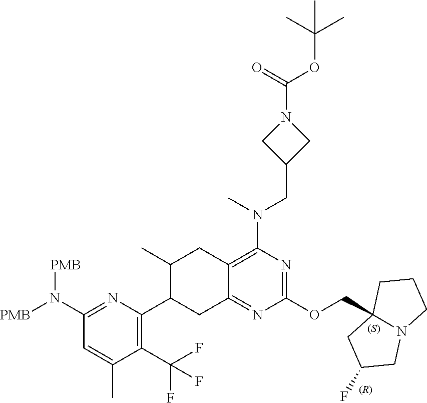

### Step 3: tert-butyl 3-(((7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate

The substitution reaction was prepared in a similar fashion to Example #65, Step 14. The resulting residue was purified by column chromatography (silica gel, 100-200 mesh, 0-100% ethyl acetate in petroleum ether) affording tert-butyl 3-(((7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (150 mg, 31.88%) as a white solid. LCMS Rt=0.670 min, m/z=917.5 [M+H]⁺.

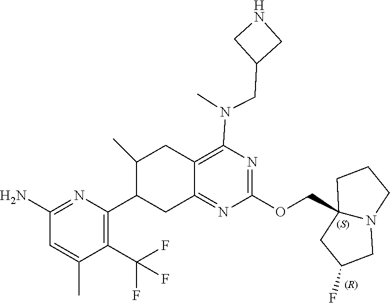

### Step 4: 7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine

The deprotection of Boc and PMB was prepared in a similar fashion to Example #65, Step 15. The crude product was purified by reverse phase HPLC (column: Phenomenex Luna 80*30 mm*3 um; mobile phase: [water (trifluroacetic acid)-acetoniitrile]; B %: 1%-30%, 8 min) affording 7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine (90 mg, 79.64%, trifluoroacetate salt) as a white solid. LCMS Rt=0.434 min, m/z=577.3 [M+H]⁺.

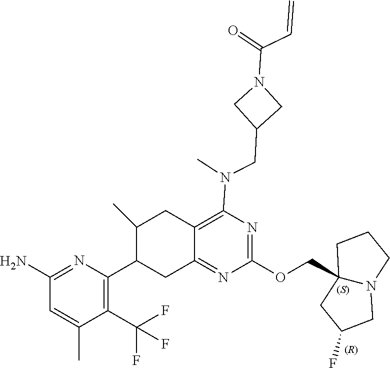

### Step 5: 1-(3-(((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one

The amide coupling reaction was prepared in a similar fashion to Example #65, Step 16. The reaction mixture was concentrated in vacuo and purified by reverse phase HPLC (column: Waters Xbridge BEH C18 100*30 mm*10 um; mobile phase: [water (NH4HCO3)-acetonitrile]; B %: 40%-60%, 8 min) affording 1-(3-(((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidin-1-yl)prop-2-en-1-one (24.21 mg, 27.67%) as a white solid: ¹H NMR (400 MHz, Acetonitrile-d3) δ 6.36-6.22 (m, 2H), 6.21-6.09 (m, 1H), 5.67-5.57 (m, 1H), 5.35-5.12 (m, 3H), 4.28 (q, J=8.6 Hz, 1H), 4.09-3.97 (m, 3H), 3.95-3.83 (m, 2H), 3.80-3.57 (m, 1H), 3.52-3.38 (m, 1H), 3.23-3.09 (m, 3H), 3.08-2.95 (m, 6H), 2.94-2.77 (m, 2H), 2.66 (dd, J=4.3, 15.8 Hz, 1H), 2.54-2.43 (m, 1H), 2.38 (q, J=3.3 Hz, 3H), 2.18-2.13 (m, 3H), 2.01 (br s, 1H), 1.93-1.75 (m, 3H), 0.77 (d, J=6.4 Hz, 3H). LCMS Rt=2.857 min, m/z=631.3 [M+H]⁺.

LCMS (5 to 95% acetonitrile in water+0.03% ammonium bicarbonate over 6 mins) retention time 2.857 min, ESI+ found [M+H]=631.3.

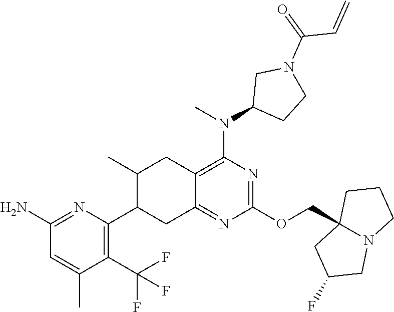 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1]

### Example 68: 1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

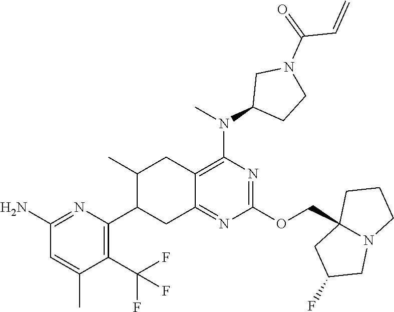 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1]

### Step 1: 1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one

The amide coupling reaction was prepared in a similar fashion to Example #65, Step 16. The residue was purified by reverse phase HPLC (column: Waters Xbridge BEH C18 100*30 mm*10 um; mobile phase: [water (NH4HCO3)-acetonitrile]; B %: 35%-65%, 8 min) affording 1-((3R)-3-((7-(6-amino-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)pyrrolidin-1-yl)prop-2-en-1-one (5.05 mg, 15.38%) as a yellow solid: ¹H NMR (400 MHz, Acetonitrile-d3) δ 6.66-6.47 (m, 1H), 6.31-6.16 (m, 2H), 5.73-5.58 (m, 1H), 5.34-5.16 (m, 3H), 4.75-4.53 (m, 1H), 4.08-4.01 (m, 1H), 3.97-3.90 (m, 1H), 3.84-3.72 (m, 1H), 3.66-3.47 (m, 1H), 3.45-3.32 (m, 1H), 3.23-3.10 (m, 3H), 3.08-2.97 (m, 2H), 2.95-2.82 (m, 5H), 2.70-2.60 (m, 1H), 2.56-2.43 (m, 1H), 2.42-2.36 (m, 3H), 2.33-2.26 (m, 2H), 2.17-2.11 (m, 2H), 2.10-2.01 (m, 3H), 1.94-1.80 (m, 3H), 0.77 (br d, J=6.5 Hz, 3H). LCMS Rt=1.012 min, m/z=631.3 [M+H]⁺.

LCMS (5 to 95% acetonitrile in water+0.03% ammonium bicarbonate over 6 mins) retention time 1.012 min, ESI+ found [M+H]=631.3.

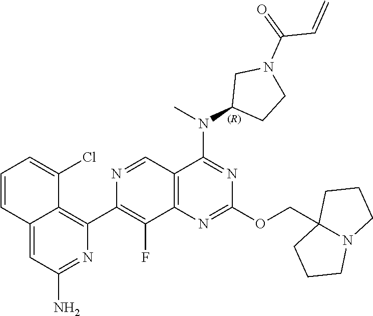

### Example 69: (R)-1-(3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one

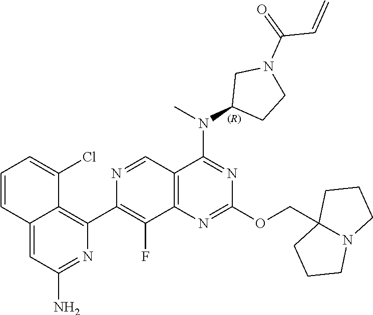

### Step 1: (R)-1-(3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The crude product was purified by reverse phase HPLC (column: Waters Xbridge Prep OBD C18 150*40 mm*10 um; mobile phase:[water (NH4HCO3)-acetonitrile]; B %: 15%-45%, 8 min) affording (R)-1-(3-((7-(3-amino-8-chloroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one (4.7 mg, 8.19%) as a yellow solid: ¹H NMR (400 MHz, Acetonitrile-d3) δ 9.19 (s, 1H), 7.66 (d, J=7.9 Hz, 1H), 7.44 (dd, J=7.4, 8.4 Hz, 1H), 7.26 (dd, J=1.0, 7.4 Hz, 1H), 6.97-6.91 (m, 1H), 6.66-6.54 (m, 1H), 6.30-6.22 (m, 1H), 5.72-5.66 (m, 1H), 5.45-5.32 (m, 1H), 5.19 (s, 2H), 4.25-4.16 (m, 2H), 4.13-3.94 (m, 1H), 3.93-3.79 (m, 1H), 3.72-3.64 (m, 1H), 3.60-3.47 (m, 1H), 3.43 (s, 3H), 3.07-2.99 (m, 2H), 2.66 (td, J=6.8, 10.1 Hz, 2H), 2.47-2.26 (m, 4H), 1.86 (qd, J=6.4, 16.0 Hz, 4H), 1.71-1.63 (m, 2H). LCMS Rt=2.267, m/z=616.3 [M+H]⁺.

LCMS (5 to 95% acetonitrile in water+0.03% ammonium bicarbonate over 6 min) retention time 2.267 min, ESI+ found [M+H]=616.3.

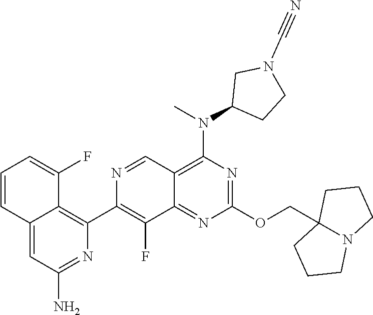 [SMILES: CN(c1nc(OCC23CCCN2CCC3)nc2c(F)c(-c3nc(N)cc4cccc(F)c34)ncc12)[C@@H]1CCN(C#N)C1]

### Example 70: (R)-3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile

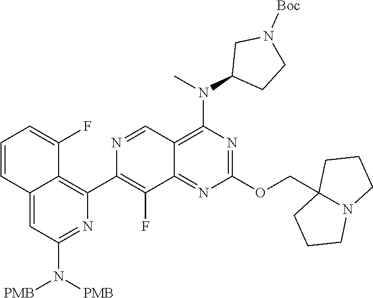

### Step 1: (R)-tert-butyl 3-((7-(3-(bis(4-methoxybenzyl)amino)-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carboxylate

The Stille reaction was prepared in a similar fashion to Example #71, Step 6, the residue was purified by reverse phase HPLC (column: Phenomenex luna C18 250*50 mm*10 um; mobile phase: [water (trifluroacetic acid)-acetoniitrile]; B %: 45%-75%, 10 min) affording (R)-tert-butyl 3-((7-(3-(bis(4-methoxybenzyl)amino)-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carboxylate (50 mg, 11.07%, trifluoroacetate salt) as a yellow solid. LCMS Rt=1.065 min, m/z=886.4 [M+H]⁺.

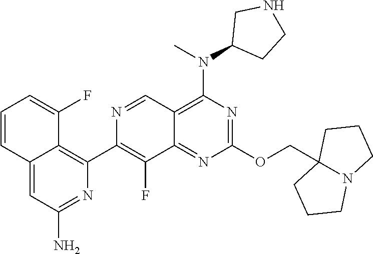 [SMILES: CN(c1nc(OCC23CCCN2CCC3)nc2c(F)c(-c3nc(N)cc4cccc(F)c34)ncc12)[C@@H]1CCNC1]

### Step 2: (R)-7-(3-amino-8-fluoroisoquinolin-1-yl)-8-fluoro-2-((hexahydro-1H-pyrrolizin-7a-yl)methoxy)-N-methyl-N-(pyrrolidin-3-yl)pyrido[4,3-d]pyrimidin-4-amine

The deprotection of Boc and PMB group was prepared in a similar fashion to Example #71, Step 7, the residue was purified by reverse phase HPLC (column: Phenomenex Luna 80*30 mm*3 um; mobile phase: [water (trifluroacetic acid)-acetoniitrile]; B %: 1%-40%, 8 min) affording (R)-7-(3-amino-8-fluoroisoquinolin-1-yl)-8-fluoro-2-((hexahydro-1H-pyrrolizin-7a-yl)methoxy)-N-methyl-N-(pyrrolidin-3-yl)pyrido[4,3-d]pyrimidin-4-amine (27 mg, 72.50%, trifluoroacetate salt) as a red solid. LCMS Rt=0.575 min, m/z=546.27 [M+H]⁺.

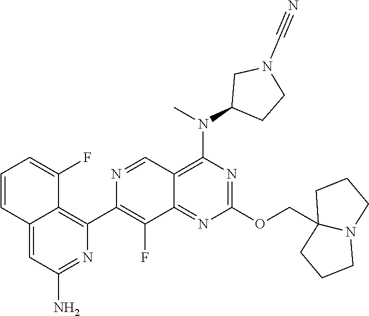 [SMILES: CN(c1nc(OCC23CCCN2CCC3)nc2c(F)c(-c3nc(N)cc4cccc(F)c34)ncc12)[C@@H]1CCN(C#N)C1]

### Step 3: (R)-3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile

The substitution reaction was prepared in a similar fashion to Example #13, Step 6. The crude product was purified by reverse phase prep-HPLC (column: Waters Xbridge BEH C18 100*30 mm*10 um; mobile phase: [water (NH4HCO3)-acetonitrile]; B %: 15%-45%, 8 min) affording (R)-3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidine-1-carbonitrile (1 mg, 3.98%) as a yellow solid: ¹H NMR (400 MHz, Acetonitrile-d3) δ 9.25-9.14 (m, 1H), 7.54-7.45 (m, 2H), 6.98-6.90 (m, 1H), 6.85 (ddd, J=2.2, 6.4, 12.6 Hz, 1H), 5.47-5.36 (m, 1H), 5.27-5.14 (m, 2H), 4.36-4.17 (m, 2H), 3.82 (br t, J=9.0 Hz, 1H), 3.69 (dt, J=3.8, 9.0 Hz, 1H), 3.53 (td, J=8.3, 16.6 Hz, 2H), 3.44 (s, 3H), 3.12-3.04 (m, 2H), 2.70 (td, J=6.6, 10.1 Hz, 2H), 2.28 (br d, J=4.8 Hz, 2H), 2.03-1.99 (m, 2H), 1.93-1.84 (m, 4H), 1.75-1.66 (m, 2H). LCMS Rt=2.245 min, m/z=571.3 [M+H]⁺.

LCMS (5 to 95% acetonitrile in water+0.03% ammonium bicarbonate over 6 mins) retention time 2.245 min, ESI+ found [M+H]=571.3.

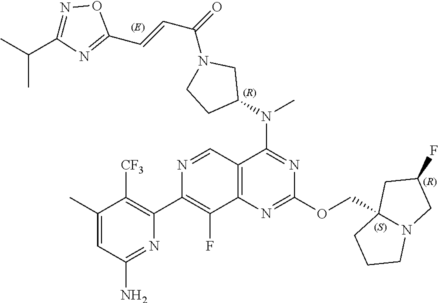

### Example 71: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

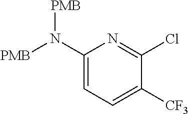

### Step 1: 6-chloro-N,N-bis(4-methoxybenzyl)-5-(trifluoromethyl)pyridin-2-amine

To a solution of 2,6-dichloro-3-(trifluoromethyl)pyridine (5 g, 23.15 mmol) in N-methylpyrrolidone (10 mL) was added 1-(4-methoxyphenyl)-N-[(4-methoxyphenyl)methyl]methanamine (5.96 g, 23.15 mmol) and triethylamine (4.68 g, 46.30 mmol), the reaction was stirred at 120° C. for 12 h. The mixture was diluted with water (20 mL) and extracted with ethyl acetate (20 mL×3). The combined organic layers were concentrated to dryness in vacuo and purified by column chromatography (silica gel, 100-200 mesh, 0-5% ethyl acetate in petroleum ether) affording 6-chloro-N,N-bis[(4-methoxyphenyl)methyl]-5-(trifluoromethyl)pyridin-2-amine (10 g, 93%) as a yellow oil. LCMS Rt=0.942 min, m/z=436.1 [M+H]⁺.

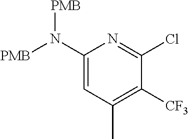

### Step 2: 6-chloro-N,N-bis(4-methoxybenzyl)-4-methyl-5-(trifluoromethyl)pyridin-2-amine

To a solution of n-butyllithium (2.5 M, 27.47 mL) in tetrahydrofuran (75 mL) was added 2,2,6,6-tetramethylpiperidine (9.70 g, 68.67 mmol) in tetrahydrofuran (30 mL) at 0° C. and stirred at 20° C. for 1.5 h. The mixture was cooled to −78° C. and 6-chloro-N,N-bis[(4-methoxyphenyl)methyl]-5-(trifluoromethyl)pyridin-2-amine (10 g, 22.89 mmol) in tetrahydrofuran (30 mL) was added dropwise to the above solution and the mixture was stirred at −78° C. for 1 h. The mixture was added iodomethane (6.50 g, 45.78 mmol) in tetrahydrofuran (30 mL) at −78° C. and stirred at −78° C. for 0.5 h under nitrogen atmosphere. The mixture was quenched by saturated ammonium chloride (100 mL) dropwise at 0° C., exacted with ethyl acetate (3×100 ml). The organic layers were concentrated in vacuo. The crude product was purified by reverse phase HPLC (column: Phenomenex C18 250*50 mm*10 um; mobile phase: [water (NH4HCO3)-acetonitrile]; B %: 65%-75%, 20 min) affording 6-chloro-N,N-bis[(4-methoxyphenyl)methyl]-4-methyl-5-(trifluoromethyl)pyridin-2-amine (3.5 g, 32%) as a yellow solid: ¹H NMR (400 MHz, Chloroform-d) δ 7.18-7.12 (m, 4H), 6.87 (d, J=8.6 Hz, 4H), 6.18-6.15 (m, 1H), 4.68 (s, 4H), 3.83-3.79 (m, 6H), 2.35-2.30 (m, 3H). LCMS Rt=0.989 min, m/z=450.1 [M+H]⁺.

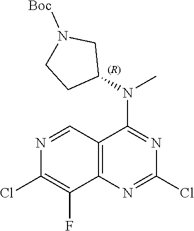

### Step 3: tert-butyl (3R)-3-[(2,7-dichloro-8-fluoro-pyrido[4,3-d]pyrimidin-4-yl)-methyl-amino]pyrrolidine-1-carboxylate

To a solution of 2,4,7-trichloro-8-fluoro-pyrido[4,3-d]pyrimidine (7 g, 27.73 mmol) in N,N-dimethylformaldehyde (15 mL) was added tert-butyl (3R)-3-(methylamino)pyrrolidine-1-carboxylate (4.44 g, 22.18 mmol) and N-ethyl-N-isopropylpropan-2-amine (10.75 g, 83.18 mmol) at 0° C. and the mixture was stirred 25° C. for 12 h under nitrogen atmosphere. The mixture was diluted with water (50 mL) and extracted with ethyl acetate (3×50 mL). The combined organic layers were dried over sodium sulphate and concentrated in vacuo affording tert-butyl (3R)-3-[(2,7-dichloro-8-fluoro-pyrido[4,3-d]pyrimidin-4-yl)-methyl-amino]pyrrolidine-1-carboxylate (13 g, crude) as a brown solid used in the next step without further purification. LCMS Rt=0.869 min, m/z=415.1 [M+H]⁺.

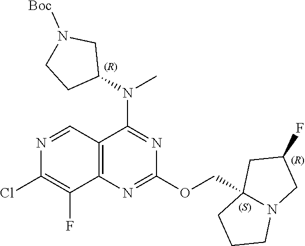

### Step 4: (R)-tert-butyl 3-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

To a solution of tert-butyl (3R)-3-[(2,7-dichloro-8-fluoro-pyrido[4,3-d]pyrimidin-4-yl)-methyl-amino]pyrrolidine-1-carboxylate (5.9 g, 14.17 mmol) in dioxane (70 mL) was added [(2R,8S)-2-fluoro-1,2,3,5,6,7-hexahydropyrrolizin-8-yl]methanol (6.77 g, 42.52 mmol) and N-ethyl-N-isopropylpropan-2-amine (7.33 g, 56.69 mmol). Then the mixture was stirred at 100° C. for 12 h under nitrogen atmosphere. The reaction mixture was concentrated in vacuo. The residue was purified by column chromatography (silica gel, 100-200 mesh, 30-50% ethyl acetate in petroleum ether) affording (R)-tert-butyl 3-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (5.7 g, 74%) as a yellow solid. LCMS Rt=0.655 min, m/z=538.2 [M+H]⁺.

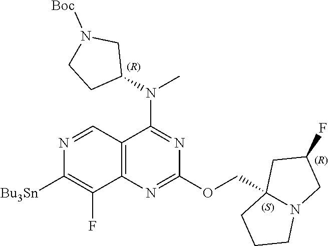

### Step 5: (R)-tert-butyl 3-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)pyrrolidine-1-carboxylate

A mixture of tributyl(tributylstannyl)stannane (4.73 g, 8.16 mmol), (R)-tert-butyl 3-((7-chloro-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (57.22 mg, 204.05 umol), lithium chloride (432.48 mg, 10.20 mmol) and (1E,4E)-1,5-diphenylpenta-1,4-dien-3-one;palladium (373.70 mg, 408.10 umol) in dioxane (200 mL) was degassed and purged with nitrogen for 3 times, and then the mixture was stirred at 110° C. for 12 h under nitrogen atmosphere. The reaction mixture was concentrated in vacuo. The crude product was purified by column chromatography (silica gel, 100-200 mesh, 2%-100% ethyl acetate in petroleum ether) affording (R)-tert-butyl 3-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)pyrrolidine-1-carboxylate (3.3 g, 74%) as a yellow oil. LCMS Rt=0.757 min, m/z=794.4 [M+H]⁺.

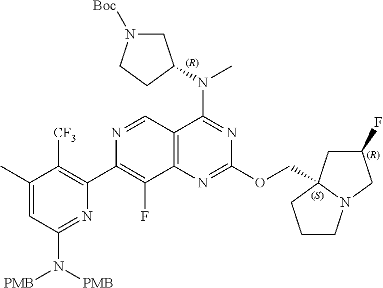

### Step 6: (R)-tert-butyl 3-((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate

A mixture of (R)-tert-butyl 3-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)pyrrolidine-1-carboxylate (2.22 g, 4.91 mmol), palladium;tritertbutylphosphane (209.29 mg, 409.52 umol) in N-methyl-2-pyrrolidone (50 mL) was degassed and purged with nitrogen for 3 times and then the mixture was stirred at 100° C. for 1 h under nitrogen atmosphere. The reaction mixture was diluted with water (30 mL) and extracted with ethyl acetate (4×20 mL). The combined organic layers were dried over sodium sulphate and concentrated in vacuo. The residue was purified by column chromatography (silica gel, 100-200 mesh, 2%-100% ethyl acetate in petroleum ether) affording (R)-tert-butyl 3-((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (3 g, 80%) as a yellow oil. LCMS Rt=0.798 min, m/z=918.4 [M+H]⁺.

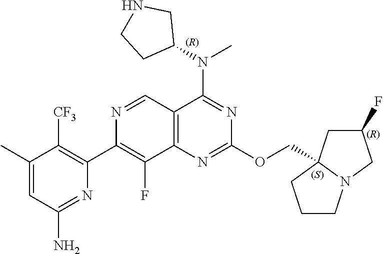

### Step 7: 7-(6-amino-4-methyl-3-(trifluoromethyl)pyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine

To a solution of tert-butyl (R)-tert-butyl 3-((7-(6-(bis(4-methoxybenzyl)amino)-4-methyl-3-(trifluoromethyl)pyridin-2-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidine-1-carboxylate (3 g, 3.26 mmol) in trifluoroacetate (2 mL). The mixture was stirred at 80° C. for 20 min. The reaction mixture was concentrated in vacuo. The residue was purified by reverse phase HPLC (column: Phenomenex luna C18 (250*70 mm,15 um); mobile phase: [water (trifluroacetic acid)-acetoniitrile]; B %: 8%-36%, 22 min) affording 7-(6-amino-4-methyl-3-(trifluoromethyl)pyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (1.5 g, 66%, trifluoroacetate salt) as a white solid. LCMS Rt=1.066 min, m/z=578.3 [M+H]⁺.

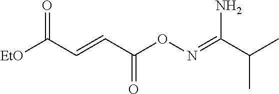 [SMILES: CCOC(=O)/C=C/C(=O)O/N=C(\N)C(C)C]

### Step 8: (E)-ethyl 4-(((Z)-(1-amino-2-methylpropylidene)amino)oxy)-4-oxobut-2-enoate

To a solution of 04-(2,5-dioxopyrrolidin-1-yl) 01-ethyl (E)-but-2-enedioate (15 g, 62.19 mmol) and N-hydroxy-2-methyl-propanamidine (6.35 g, 62.19 mmol) in dioxane (150 mL) was added potassium carbonate (25.79 g, 186.57 mmol) at 0° C., the mixture was stirred at 25° C. for 12 hr. The reaction mixture was diluted with water (50 mL) and extracted with ethyl acetate (4×100 mL). The combined organic layers were dried over sodium sulphate and concentrated in vacuo affording (E)-ethyl 4-(((Z)-(1-amino-2-methylpropylidene)amino)oxy)-4-oxobut-2-enoate (15 g, crude), used in next step without further purification. LCMS Rt=0.545 min, m/z=228.1 [M+H]⁺.

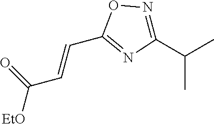

### Step 9: (E)-ethyl 3-(3-isopropyl-1,2,4-oxadiazol-5-yl)acrylate

To a solution of (E)-ethyl 4-(((Z)-(1-amino-2-methylpropylidene)amino)oxy)-4-oxobut-2-enoate (15 g, 65.72 mmol) in dioxane (150 mL) was added 2,4,6-tripropyl-1,3,5,2,4,6-trioxatriphosphinane 2,4,6-trioxide (131.44 mmol, 78.17 mL, 50% purity, in ethyl acetate), the mixture was stirred at 90° C. for 2 hr. The reaction mixture was diluted with water (30 mL) and extracted with ethyl acetate (4×50 mL). The combined organic layers were dried over sodium sulphate and concentrated in vacuo affording (E)-ethyl 3-(3-isopropyl-1,2,4-oxadiazol-5-yl)acrylate (15 g, crude), used in next step without further purification. LCMS Rt=0.740 min, m/z=210.1 [M+H]⁺.

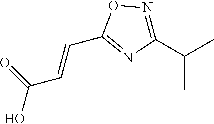

### Step 10: (E)-3-(3-isopropyl-1,2,4-oxadiazol-5-yl)acrylic acid

To a solution of (E)-ethyl 3-(3-isopropyl-1,2,4-oxadiazol-5-yl)acrylate (10 g, 47.57 mmol) in THF (100 mL) and H₂O (50 mL) was added LiOH·H₂O (2.40 g, 57.08 mmol) at 0° C. The mixture was stirred at 0° C. for 1 h. The reaction mixture was quenched with 1N HCl (100 mL) at 0° C. and extracted with dichloromethane (3×100 mL). The combined organic layers were dried over sodium sulphate and concentrated in vacuo affording (E)-3-(3-isopropyl-1,2,4-oxadiazol-5-yl)acrylic acid (8 g, 92.32%) as a white solid: ¹H NMR (400 MHz, Chloroform-d) δ 11.85-11.36 (m, 1H), 7.53 (d, J=16.0 Hz, 1H), 6.97 (d, J=16.0 Hz, 1H), 3.10 (s, 1H), 1.30 (d, J=7.0 Hz, 6H).

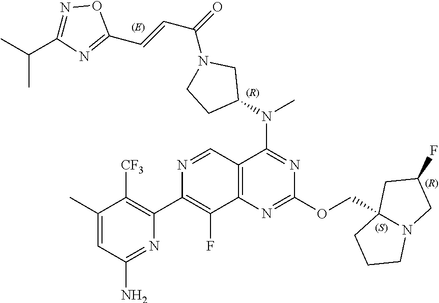

### Step 11: (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

To a solution of 7-(6-amino-4-methyl-3-(trifluoromethyl)pyridin-2-yl)-8-fluoro-2-(((2R,7aS)-2-fluorohexahydro-1H-pyrrolizin-7a-yl)methoxy)-N-methyl-N—((R)-pyrrolidin-3-yl)pyrido[4,3-d]pyrimidin-4-amine (350 mg, 505.34 umol, trifluoroacetate salt), N,N-Diisopropylethylamine (195.94 mg, 1.52 mmol) and (E)-3-(3-isopropyl-1,2,4-oxadiazol-5-yl)prop-2-enoic acid (73.65 mg, 404.27 umol) in dichloromethane (4 mL) was added 2,4,6-tripropyl-1,3,5,2,4,6-trioxatriphosphinane 2,4,6-trioxide (1.01 mmol, 601.08 uL, 50% purity, in ethyl acetate) at −10° C. The reaction mixture was concentrated in vacuo. The mixture was stirred at −10° C. for 0.5 h, the residue was purified by reverse phase HPLC(column: Waters Xbridge BEH C18 100*30 mm*10 um; mobile phase: [water (NH4HCO3)-acetonitrile]; B %: 30%-70%, 10 min) affording (E)-1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (51.24 mg, 14%) as a yellow solid: ¹H NMR (400 MHz, Acetonitrile-d3) δ 9.17-9.08 (m, 1H), 7.55-7.34 (m, 2H), 6.63-6.49 (m, 1H), 5.54 (br d, J=5.3 Hz, 2H), 5.41-5.19 (m, 2H), 4.29-4.09 (m, 3H), 4.05-3.95 (m, 1H), 3.92-3.70 (m, 2H), 3.66-3.46 (m, 1H), 3.41 (s, 3H), 3.20-3.07 (m, 4H), 2.99-2.84 (m, 1H), 2.41-2.28 (m, 3H), 2.28-2.17 (m, 2H), 2.15-2.01 (m, 2H), 1.93-1.81 (m, 3H), 1.34 (dd, J=7.0, 9.4 Hz, 6H). LCMS Rt=2.828 min, m/z=742.3 [M+H]⁺.

LCMS (5 to 95% acetonitrile in water+0.1% trifluoroacetic acid over 6 mins) retention time 2.828 min, ESI+ found [M+H]=742.3.

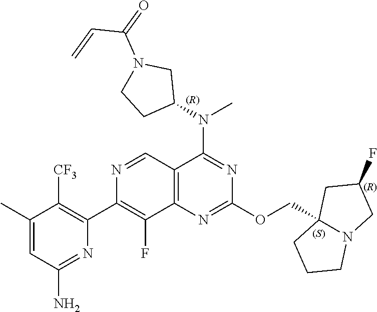

### Example 72: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

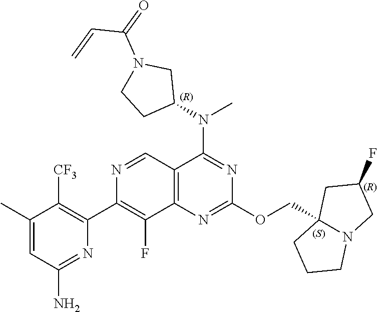

### Step 1: 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The residue was purified by reverse phase HPLC (column: Waters Xbridge Prep OBD C18 150*40 mm*10 um; mobile phase: [water (NH₄HCO₃)-acetonitrile]; B %: 25%-55%, 8 min) affording 1-((R)-3-((7-(6-amino-4-methyl-3-(trifluoromethyl)pyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (12 mg, 19%) as a white solid: ¹H NMR (400 MHz, Acetonitrile-d3) δ 9.15 (d, J=1.6 Hz, 1H), 6.66-6.53 (m, 2H), 6.30-6.22 (m, 1H), 5.69 (ddd, J=2.3, 7.7, 10.2 Hz, 1H), 5.48 (s, 2H), 5.42-5.20 (m, 2H), 4.26-4.20 (m, 1H), 4.17-4.12 (m, 1H), 4.09-3.79 (m, 2H), 3.71-3.62 (m, 1H), 3.59-3.44 (m, 1H), 3.40 (s, 3H), 3.21-3.11 (m, 2H), 3.08 (s, 1H), 2.95-2.88 (m, 1H), 2.46 (d, J=1.4 Hz, 3H), 2.42-2.36 (m, 1H), 2.34-2.20 (m, 2H), 2.12 (d, J=2.6 Hz, 1H), 2.10-2.02 (m, 1H), 1.95-1.81 (m, 3H). LCMS Rt=2.512 min, m/z=632.3 [M+H]⁺.

LCMS (5 to 95% acetonitrile in water+0.1% trifluoroacetic acid over 6 mins) retention time 2.512 min, ESI+ found [M+H]=632.3.

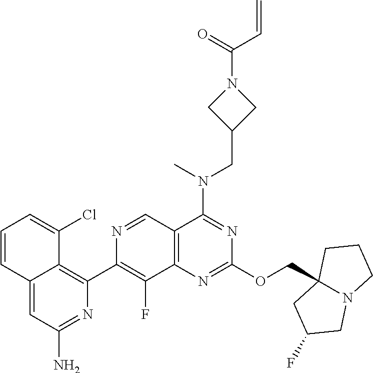 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1]

### Example 73: 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one

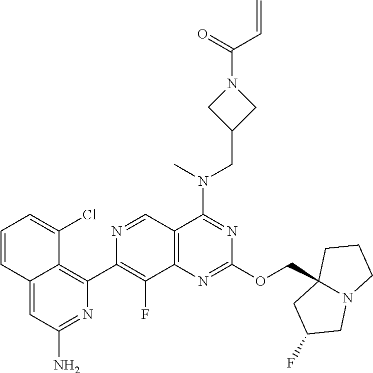 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1]

### Step 1: 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one

The amide coupling reaction was prepared in a similar fashion to Example #71, Step 11. The crude product was purified by reverse phase prep-HPLC (column: Waters Xbridge Prep OBD C18 150*40 mm*10 um; mobile phase: [water (NH4HCO3)-acetonitrile]; B %: 20%-50%, 8 min) affording 1-(3-(((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)methyl)azetidin-1-yl)prop-2-en-1-one (12.7 mg, 20.00%) as a yellow solid: ¹H NMR (400 MHz, Acetonitrile-d3) δ 9.22 (s, 1H), 7.66-7.61 (m, 1H), 7.45-7.39 (m, 1H), 7.28-7.20 (m, 1H), 6.95-6.90 (m, 1H), 6.34-6.25 (m, 1H), 6.20-6.13 (m, 1H), 5.64 (dd, J=2.3, 10.2 Hz, 1H), 5.41-5.28 (m, 1H), 5.20-5.08 (m, 3H), 4.39-4.30 (m, 1H), 4.19-4.10 (m, 5H), 3.88-3.81 (m, 1H), 3.57 (s, 3H), 3.20-3.12 (m, 3H), 3.09-3.06 (m, 1H), 2.94-2.88 (m, 1H), 2.05 (br d, J=7.6 Hz, 3H), 1.87-1.77 (m, 3H). LCMS Rt=2.450 min, m/z=634.2 [M+H]⁺.

LCMS (5 to 95% acetonitrile in water+0.1% trifluoroacetic acid over 6 mins) retention time 2.450 min, ESI+ found [M+H]=634.2.

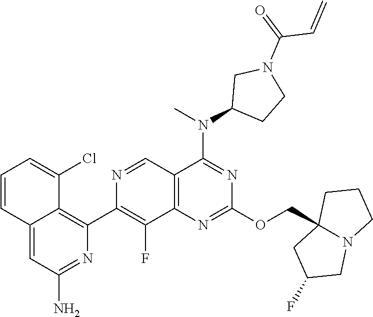 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1]

### Example 74: 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one

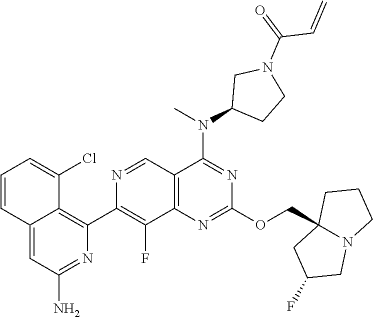 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1]

### Step 1: 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The crude was purified by reverse phase HPLC (column: Waters Xbridge Prep OBD C18 150*40 mm*10 um; mobile phase:[water (NH₄HCO₃)-acetonitrile]; B %: 20%-50%, 8 min) affording 1-((R)-3-((7-(3-amino-8-chloroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one (34.18 mg, 20.60%) as a yellow amorphous solid: ¹H NMR (400 MHz, Acetonitrile-d3) δ 9.17 (s, 1H), 7.63 (d, J=8.3 Hz, 1H), 7.41 (t, J=7.9 Hz, 1H), 7.23 (dd, J=0.9, 7.3 Hz, 1H), 6.95-6.90 (m, 1H), 6.64-6.52 (m, 1H), 6.27-6.19 (m, 1H), 5.69-5.62 (m, 1H), 5.44-5.20 (m, 2H), 5.18-5.11 (m, 2H), 4.27-4.02 (m, 3H), 3.99-3.77 (m, 2H), 3.71-3.62 (m, 1H), 3.59-3.48 (m, 1H), 3.41 (s, 3H), 3.16-3.08 (m, 2H), 2.94-2.83 (m, 1H), 2.42-2.24 (m, 3H), 2.08-2.01 (m, 2H), 1.90-1.77 (m, 3H). LCMS Rt=1.929 min, m/z=634.2 [M+H]⁺.

LCMS (5 to 95% acetonitrile in water+0.03% ammonium bicarbonate over 6 mins) retention time 1.929 min, ESI+ found [M+H]=634.2.

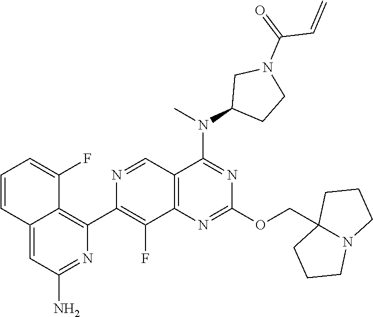 [SMILES: C=CC(=O)N1CC[C@@H](N(C)c2nc(OCC34CCCN3CCC4)nc3c(F)c(-c4nc(N)cc5cccc(F)c45)ncc23)C1]

### Example 75: (R)-1-(3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one

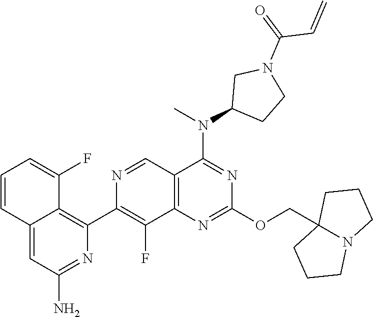 [SMILES: C=CC(=O)N1CC[C@@H](N(C)c2nc(OCC34CCCN3CCC4)nc3c(F)c(-c4nc(N)cc5cccc(F)c45)ncc23)C1]

### Step 1: (R)-1-(3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The crude product was purified by reverse phase prep-HPLC (column: Waters Xbridge BEH C18 100*30 mm*10 um; mobile phase: [water (NH4HCO3)-acetonitrile]; B %: 1%-50%, 8 min) affording (R)-1-(3-((7-(3-amino-8-fluoroisoquinolin-1-yl)-8-fluoro-2-((hexahydro-1H-pyrrolizin-7a-yl)methoxy)pyrido[4,3-d]pyrimidin-4-yl)(methyl)amino)pyrrolidin-1-yl)prop-2-en-1-one (2.24 mg, 10.19%) as a yellow solid: ¹H NMR (400 MHz, Acetonitrile-d3) δ 9.21 (d, J=3.6 Hz, 1H), 7.59-7.38 (m, 2H), 6.93 (d, J=2.0 Hz, 1H), 6.89-6.79 (m, 1H), 6.61 (dt, J=10.3, 16.2 Hz, 1H), 6.34-6.17 (m, 1H), 5.70 (ddd, J=2.2, 7.8, 10.0 Hz, 1H), 5.47-5.31 (m, 1H), 5.20 (br s, 2H), 4.18 (br d, J=4.1 Hz, 2H), 4.04-3.79 (m, 1H), 3.73-3.63 (m, 1H), 3.61-3.48 (m, 1H), 3.43 (s, 3H), 3.05-2.96 (m, 3H), 2.67-2.59 (m, 2H), 2.45-2.36 (m, 1H), 2.35-2.25 (m, 2H), 1.91-1.77 (m, 5H), 1.65 (td, J=7.4, 12.1 Hz, 2H). LCMS Rt=2.170 min, m/z=600.3 [M+H]⁺.

LCMS (5 to 95% acetonitrile in water+0.03% ammonium bicarbonate over 6 mins) retention time 2.170 min, ESI+ found [M+H]=600.3.

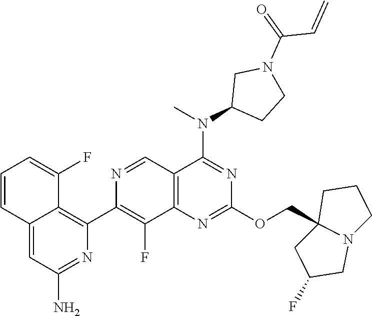 [SMILES: C=CC(=O)N1CC[C@@H](N(C)c2nc(OC[C@@]34CCCN3C[C@H](F)C4)nc3c(F)c(-c4nc(N)cc5cccc(F)c45)ncc23)C1]

### Example 76: 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one

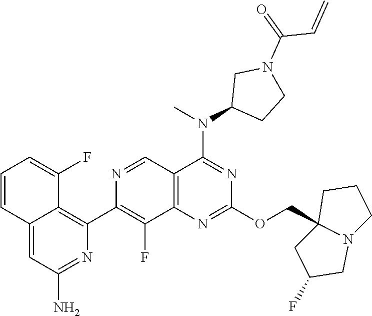 [SMILES: C=CC(=O)N1CC[C@@H](N(C)c2nc(OC[C@@]34CCCN3C[C@H](F)C4)nc3c(F)c(-c4nc(N)cc5cccc(F)c45)ncc23)C1]

### Step 1: 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one

The amide coupling reaction was prepared in a similar fashion to Example #71, Step 11. The crude was purified by reverse phase HPLC (column: Waters Xbridge BEH C18 100*30 mm*10 um; mobile phase: [water (NH4HCO3)-acetonitrile]; B %: 30%-60%, 10 min) affording 1-((R)-3-((7-(3-amino-8-fluoroisoquinolin-1-yl)-8-fluoro-2-(((2R,7aS)-2-fluorohexahydro-1H-pyrrolizin-7a-yl)methoxy)pyrido[4,3-d]pyrimidin-4-yl)(methyl)amino)pyrrolidin-1-yl)prop-2-en-1-one (40 mg, 51.65%) as a white solid: ¹H NMR (400 MHz, Acetonitrile-d3) δ 9.24-9.18 (m, 1H), 7.53-7.43 (m, 2H), 6.92 (d, J=2.5 Hz, 1H), 6.84 (ddd, J=2.3, 6.2, 12.5 Hz, 1H), 6.60 (dt, J=10.3, 16.9 Hz, 1H), 6.26 (ddd, J=2.4, 4.7, 16.8 Hz, 1H), 5.69 (ddd, J=2.1, 8.1, 10.3 Hz, 1H), 5.39-5.32 (m, 1H), 5.21 (br s, 2H), 4.26-4.19 (m, 1H), 4.17-4.10 (m, 1H), 4.00-3.89 (m, 1H), 3.89-3.79 (m, 1H), 3.71-3.62 (m, 1H), 3.60-3.44 (m, 1H), 3.43 (s, 3H), 3.19-3.12 (m, 2H), 3.09 (s, 1H), 2.95-2.88 (m, 1H), 2.39 (q, J=8.6 Hz, 1H), 2.34-2.27 (m, 1H), 2.25 (br d, J=4.4 Hz, 1H), 2.13 (d, J=2.9 Hz, 1H), 2.11-2.03 (m, 1H), 1.96-1.79 (m, 4H). LCMS Rt=2.428 min, m/z=618.3 [M+H]⁺.

LCMS (5 to 95% acetonitrile in water+0.03% ammonium bicarbonate over 6 mins) retention time 2.428 min, ESI+ found [M+H]=618.3.

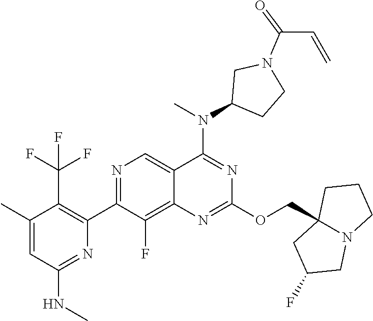 [SMILES: C=CC(=O)N1CC[C@@H](N(C)c2nc(OC[C@@]34CCCN3C[C@H](F)C4)nc3c(F)c(-c4nc(NC)cc(C)c4C(F)(F)F)ncc23)C1]

### Example 71 (alternative): 1-((R)-3-((8-fluoro-2-(((2R,7aS)-2-fluorotetrahydro-1H-pyrrolizin-7a(5H)-yl)methoxy)-7-(4-methyl-6-(methylamino)-3-(trifluoromethyl)pyridin-2-yl)pyrido[4,3-d]pyrimidin-4-yl)(methyl)amino)pyrrolidin-1-yl)prop-2-en-1-one

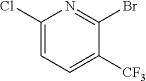 [SMILES: FC(F)(F)c1ccc(Cl)nc1Br]

### Step 1: 2-bromo-6-chloro-3-(trifluoromethyl)pyridine

A mixture of phosphoryl tribromide (21.77 g, 75.93 mmol) and 2-chloro-1-oxido-5-(trifluoromethyl)pyridin-1-ium (5 g, 25.31 mmol) was stirred at 80° C. for 12 h under nitrogen atmosphere. The mixture was then cooled to room temperature, diluted with Na₂CO₃ (20 mL) and extracted with EtOAc (3×50 mL). The combined organic layers were dried over sodium sulfate and concentrated in vacuo. The crude residue was purified by column chromatography (silica gel, 100-200 mesh, 0-50% EtOAc in petroleum ether) affording 2-bromo-6-chloro-3-(trifluoromethyl)pyridine (1.2 g, 85.37%): ¹H NMR (400 MHz, CD₃CN) δ 7.96 (d, J=8.2 Hz, 1H), 7.75 (d, J=8.2 Hz, 1H).

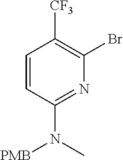

### Step 2: 6-bromo-N-(4-methoxybenzyl)-N-methyl-5-(trifluoromethyl)pyridin-2-amine

To a solution of 2-bromo-6-chloro-3-(trifluoromethyl)pyridine (1.2 g, 4.61 mmol) in NMP (15 mL) was added NEt₃ (932.49 mg, 9.22 mmol) and 1-(4-methoxyphenyl)-N-methyl-methanamine (836.03 mg, 5.53 mmol). The resulting mixture was then stirred at 120° C. for 6 h. After this time, the mixture was cooled to room temperature, diluted with water (50 mL) and extracted with EtOAc (3×50 mL). The combined organic layers were dried over sodium sulfate and concentrated in vacuo affording 6-bromo-N-(4-methoxybenzyl)-N-methyl-5-(trifluoromethyl)pyridin-2-amine (2 g, crude) as a yellow oil which was used in next step without any further purification. LCMS Rt=0.902 min, m/z=375.0 [M+H]⁺.

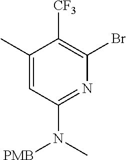

### Step 3: 6-bromo-N-(4-methoxybenzyl)-N,4-dimethyl-5-(trifluoromethyl)pyridin-2-amine

To a solution of n-BuLi (2.5 M in hexanes, 2.56 mL) in THF (10 mL) was added a solution of TMP (903.58 mg, 6.40 mmol) in THF (0.5 mL) at −70° C. The resulting mixture was then warmed to 20° C. and stirring was continued at this temperature for 1.5 h. The reaction mixture was then cooled to −78° C. and a solution of 6-bromo-N-[(4-methoxyphenyl)methyl]-N-methyl-5-(trifluoromethyl)pyridin-2-amine (800 mg, 2.13 mmol) in THF (0.5 mL) was added. Stirring was continued at this temperature for 1 h, then a solution of methyl iodide (907.96 mg, 6.40 mmol) in THF (0.5 mL) was added. After stirring for an additional 30 min at −78° C., the reaction mixture was warmed to 0° C. and quenched with saturated ammonium chloride (100 mL). The layers were separated and the aqueous phase was then further extracted with EtOAc (3×100 mL). The combined organic layers were dried over sodium sulfate and concentrated in vacuo. The crude residue was purified by column chromatography (silica gel, 100-200 mesh, 0-60% EtOAc in petroleum ether) affording 6-bromo-N-(4-methoxybenzyl)-N,4-dimethyl-5-(trifluoromethyl)pyridin-2-amine (700 mg, 84.35%) as a purple gum: ¹H NMR (400 MHz, CDCl₃) δ 7.17 (d, J=8.8 Hz, 2H), 6.89-6.85 (m, 2H), 6.19 (s, 1H), 4.72 (s, 2H), 3.83-3.79 (m, 3H), 3.04 (s, 3H), 2.39 (q, J=3.0 Hz, 3H). LCMS Rt=0.932 min, m/z=389.0 [M+H]⁺.

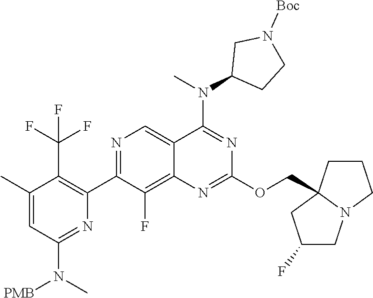

### Step 4: (R)-tert-butyl 3-((8-fluoro-2-(((2R,7aS)-2-fluorohexahydro-1H-pyrrolizin-7a-yl)methoxy)-7-(6-((4-methoxybenzyl)(methyl)amino)-4-methyl-3-(trifluoromethyl)pyridin-2-yl)pyrido[4,3-d]pyrimidin-4-yl)(methyl)amino)pyrrolidine-1-carboxylate

The Stille reaction was performed in a similar fashion to Example #71, Step 6. The crude residue was purified by column chromatography (silica gel, 100-200 mesh, 0-60% EtOAc in petroleum ether) affording (R)-tert-butyl 3-((8-fluoro-2-(((2R,7aS)-2-fluorohexahydro-1H-pyrrolizin-7a-yl)methoxy)-7-(6-((4-methoxybenzyl)(methyl)amino)-4-methyl-3-(trifluoromethyl)pyridin-2-yl)pyrido[4,3-d]pyrimidin-4-yl)(methyl)amino)pyrrolidine-1-carboxylate (75 mg, 4%) as a yellow oil. LCMS Rt=0.932 min, m/z=813.4 [M+H]⁺.

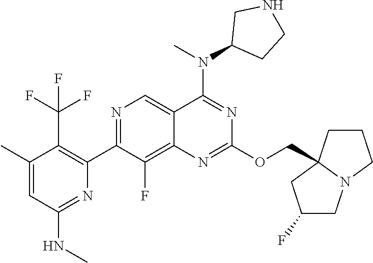 [SMILES: CNc1cc(C)c(C(F)(F)F)c(-c2ncc3c(N(C)[C@@H]4CCNC4)nc(OC[C@@]45CCCN4C[C@H](F)C5)nc3c2F)n1]

### Step 5: 8-fluoro-2-(((2R,7aS)-2-fluorohexahydro-1H-pyrrolizin-7a-yl)methoxy)-N-methyl-7-(4-methyl-6-(methylamino)-3-(trifluoromethyl)pyridin-2-yl)-N—((R)-pyrrolidin-3-yl)pyrido[4,3-d]pyrimidin-4-amine

The deprotection of Boc and PMB reaction was performed in a similar fashion to Example #71, Step 7. The reaction mixture was concentrated in vacuo affording 8-fluoro-2-(((2R,7aS)-2-fluorohexahydro-1H-pyrrolizin-7a-yl)methoxy)-N-methyl-7-(4-methyl-6-(methylamino)-3-(trifluoromethyl)pyridin-2-yl)-N—((R)-pyrrolidin-3-yl)pyrido[4,3-d]pyrimidin-4-amine (65.00 mg, crude) as a brown oil which was used in the next step without further purification. LCMS Rt=0.660 min, m/z=593.3 [M+H]⁺.

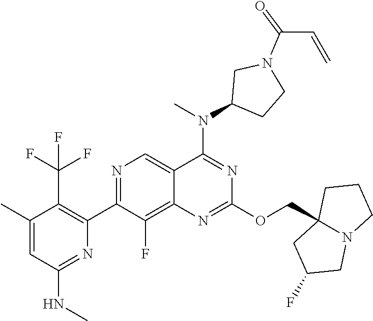 [SMILES: C=CC(=O)N1CC[C@@H](N(C)c2nc(OC[C@@]34CCCN3C[C@H](F)C4)nc3c(F)c(-c4nc(NC)cc(C)c4C(F)(F)F)ncc23)C1]

### Step 6: 1-((R)-3-((8-fluoro-2-(((2R,7aS)-2-fluorotetrahydro-1H-pyrrolizin-7a(5H)-yl)methoxy)-7-(4-methyl-6-(methylamino)-3-(trifluoromethyl)pyridin-2-yl)pyrido[4,3-d]pyrimidin-4-yl)(methyl)amino)pyrrolidin-1-yl)prop-2-en-1-one

The acylation reaction was performed in a similar fashion to Example #71, Step 11. The residue was purified by reverse phase HPLC (column: Waters Xbridge Prep OBD C18 150*40 mm*10 um; mobile phase: [water(NH₄HCO₃)-ACN]; B %: 35%-75%, 8 min) affording 1-((R)-3-((8-fluoro-2-(((2R,7aS)-2-fluorotetrahydro-1H-pyrrolizin-7a(5H)-yl)methoxy)-7-(4-methyl-6-(methylamino)-3-(trifluoromethyl)pyridin-2-yl)pyrido[4,3-d]pyrimidin-4-yl)(methyl)amino)pyrrolidin-1-yl)prop-2-en-1-one (12.4 mg, 13.60%) as a yellow solid: ¹H NMR (400 MHz, CD₃CN) δ 9.13 (d, J=1.5 Hz, 1H), 6.63-6.47 (m, 2H), 6.26-6.18 (m, 1H), 5.71-5.61 (m, 2H), 5.19 (s, 2H), 4.25-4.10 (m, 2H), 3.91 (d, J=8.0 Hz, 1H), 3.87 (s, 1H), 3.69-3.58 (m, 1H), 3.57-3.42 (m, 1H), 3.40-3.37 (m, 3H), 3.16 (d, J=7.5 Hz, 2H), 3.11-3.06 (m, 1H), 2.94-2.88 (m, 1H), 2.85-2.81 (m, 3H), 2.44 (d, J=1.5 Hz, 3H), 2.39-2.23 (m, 2H), 2.10-2.03 (m, 2H), 1.90 (m, 4H). LCMS Rt=2.653 min, m/z=647.3 [M+H]⁺.

LCMS (5 to 95% acetonitrile in water+0.03% ammonium bicarbonate over 6 mins) retention time 2.653 min, ESI+ found [M+H]⁺=647.3.

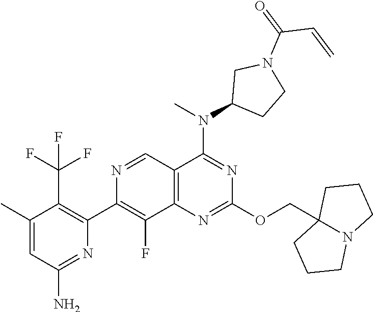 [SMILES: C=CC(=O)N1CC[C@@H](N(C)c2nc(OCC34CCCN3CCC4)nc3c(F)c(-c4nc(N)cc(C)c4C(F)(F)F)ncc23)C1]

### Example 79: (R)-1-(3-((7-(6-amino-4-methyl-3-(trifluoromethyl)pyridin-2-yl)-8-fluoro-2-((tetrahydro-1H-pyrrolizin-7a(5H)-yl)methoxy)pyrido[4,3-d]pyrimidin-4-yl)(methyl)amino)pyrrolidin-1-yl)prop-2-en-1-one

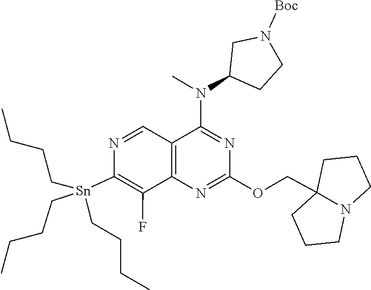 [SMILES: CCC[CH2][Sn]([CH2]CCC)([CH2]CCC)[c]1ncc2c(N(C)[C@@H]3CCN(C(=O)OC(C)(C)C)C3)nc(OCC34CCCN3CCC4)nc2c1F]

### Step 1: tert-butyl (R)-3-((8-fluoro-2-((tetrahydro-1H-pyrrolizin-7a(5H)-yl)methoxy)-7-(tributylstannyl)pyrido[4,3-d]pyrimidin-4-yl)(methyl)amino)pyrrolidine-1-carboxylate

The tin reagent reaction was performed in a similar fashion to Example #71, Step 5. The resulting crude residue was purified by column chromatography (silica gel, 100-200 mesh, 0-100% EtOAc in petroleum ether) affording tert-butyl (R)-3-((8-fluoro-2-((tetrahydro-1H-pyrrolizin-7a(5H)-yl)methoxy)-7-(tributylstannyl)pyrido[4,3-d]pyrimidin-4-yl)(methyl)amino)pyrrolidine-1-carboxylate (80 mg, 35.83%) as a white solid. LCMS Rt=0.662 min, m/z=777.4 [M+H]⁺.

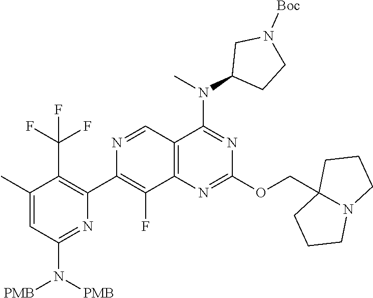

### Step 2: tert-butyl (R)-3-((7-(6-(bis(4-methoxybenzyl)amino)-4-methyl-3-(trifluoromethyl)pyridin-2-yl)-8-fluoro-2-((tetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The resulting residue was purified by reverse phase HPLC (column: Phenomenex Luna 80*30 mm*3 um; mobile phase: [water(TFA)-ACN]; B %: 40%-70%, 8 min) affording tert-butyl (R)-3-((7-(6-(bis(4-methoxybenzyl)amino)-4-methyl-3-(trifluoromethyl)pyridin-2-yl)-8-fluoro-2-((tetrahydro-1H-pyrrolizin-7a(5H)-yl)methoxy)pyrido[4,3-d]pyrimidin-4-yl)(methyl)amino)pyrrolidine-1-carboxylate (10 mg, 43.04%) as a white solid. LCMS Rt=2.642 min, m/z=901.4 [M+H]⁺.

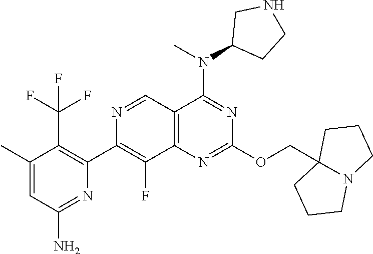 [SMILES: Cc1cc(N)nc(-c2ncc3c(N(C)[C@@H]4CCNC4)nc(OCC45CCCN4CCC5)nc3c2F)c1C(F)(F)F]

### Step 3: (R)-7-(6-amino-4-methyl-3-(trifluoromethyl)pyridin-2-yl)-8-fluoro-N-methyl-N-(pyrrolidin-3-yl)-2-((tetrahydro-1H-pyrrolizin-7a(5H)-yl)methoxy)pyrido[4,3-d]pyrimidin-4-amine

The deprotection of Boc and PMB reaction was performed in a similar fashion to Example #71, Step 7. The mixture was concentrated in vacuo affording (R)-7-(6-amino-4-methyl-3-(trifluoromethyl)pyridin-2-yl)-8-fluoro-N-methyl-N-(pyrrolidin-3-yl)-2-((tetrahydro-1H-pyrrolizin-7a(5H)-yl)methoxy)pyrido[4,3-d]pyrimidin-4-amine (10 mg, crude, trifluoroacetic salt) as a yellow oil used in next step without any further purification. LCMS Rt=0.554 min, m/z=561.3 [M+H]⁺.

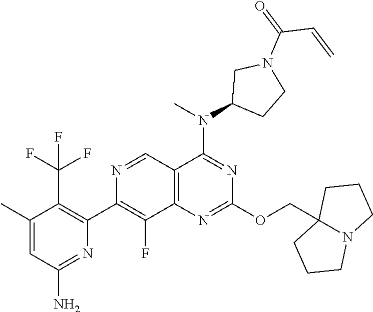 [SMILES: C=CC(=O)N1CC[C@@H](N(C)c2nc(OCC34CCCN3CCC4)nc3c(F)c(-c4nc(N)cc(C)c4C(F)(F)F)ncc23)C1]

### Step 4: (R)-1-(3-((7-(6-amino-4-methyl-3-(trifluoromethyl)pyridin-2-yl)-8-fluoro-2-((tetrahydro-1H-pyrrolizin-7a(5H)-yl)methoxy)pyrido[4,3-d]pyrimidin-4-yl)(methyl)amino)pyrrolidin-1-yl)prop-2-en-1-one

The acylation reaction was performed in a similar fashion to Example #71, Step 11. The reaction mixture was concentrated in vacuo and the residue was purified by reverse phase HPLC (column: Waters Xbridge BEH C18 100*30 mm*10 um; mobile phase: [water(NH₄HCO₃)-ACN]; B %: 20%-50%, 8 min) affording (R)-1-(3-((7-(6-amino-4-methyl-3-(trifluoromethyl)pyridin-2-yl)-8-fluoro-2-((tetrahydro-1H-pyrrolizin-7a(5H)-yl)methoxy)pyrido[4,3-d]pyrimidin-4-yl)(methyl)amino)pyrrolidin-1-yl)prop-2-en-1-one (1 mg, 10.46%) as a white solid: ¹H NMR (400 MHz, CD₃CN) δ 9.17 (s, 1H), 6.60 (br s, 1H), 6.26 (d, J=16.8 Hz, 1H), 5.70 (t, J=8.4 Hz, 1H), 5.50 (s, 2H), 5.43-5.32 (m, 1H), 4.37 (m, 2H), 4.14-4.02 (m, 1H), 3.99-3.79 (m, 2H), 3.67 (br s, 1H), 3.60-3.47 (m, 1H), 3.42 (s, 3H), 3.26 (m, 2H), 2.87-2.79 (m, 2H), 2.42-2.32 (m, 9H), 1.89-1.73 (m, 4H). LCMS Rt=2.427 min, m/z=615.3 [M+H]⁺.

LCMS (5 to 95% acetonitrile in water+0.03% ammonium bicarbonate over 6 mins) retention time 2.427 min, ESI+ found [M+H]⁺=615.3.

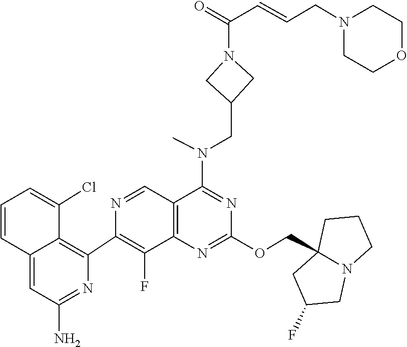 [SMILES: CN(CC1CN(C(=O)/C=C/CN2CCOCC2)C1)c1nc(OC[C@@]23CCCN2C[C@H](F)C3)nc2c(F)c(-c3nc(N)cc4cccc(Cl)c34)ncc12]

### Example 80: (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one

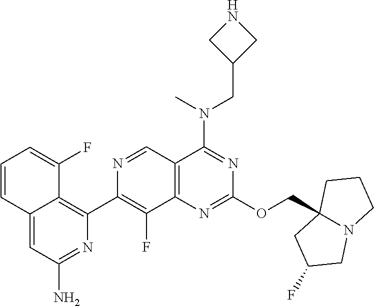 [SMILES: CN(CC1CNC1)c1nc(OC[C@@]23CCCN2C[C@H](F)C3)nc2c(F)c(-c3nc(N)cc4cccc(F)c34)ncc12]

### Step 1: 7-(3-amino-8-fluoroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The deprotection of Boc and PMB reaction was performed in a similar fashion to Example #71, Step 7. The mixture was concentrated to dryness in vacuo affording 7-(3-amino-8-fluoroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (50 mg, crude, trifluoroacetic salt) as a yellow solid used in next step without any further purification. LCMS Rt=0.440 min, m/z=565.3 [M+H]⁺.

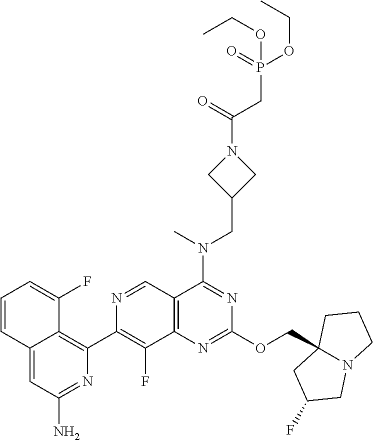 [SMILES: CCOP(=O)(CC(=O)N1CC(CN(C)c2nc(OC[C@@]34CCCN3C[C@H](F)C4)nc3c(F)c(-c4nc(N)cc5cccc(F)c45)ncc23)C1)OCC]

### Step 2: diethyl (2-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-2-oxoethyl)phosphonate

The amide coupling reaction was performed in a similar fashion to Example #2, Step 5. The crude product was purified by reverse phase HPLC (column: Waters Xbridge BEH C18 100*30 mm*10 um; mobile phase: [water(NH₄HCO₃)-ACN]; B %: 15%-55%, 8 min) affording diethyl (2-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-2-oxoethyl)phosphonate (15 mg, 45.31%) as a yellow solid. LCMS Rt=1.783 min, m/z=743.3 [M+H]⁺.

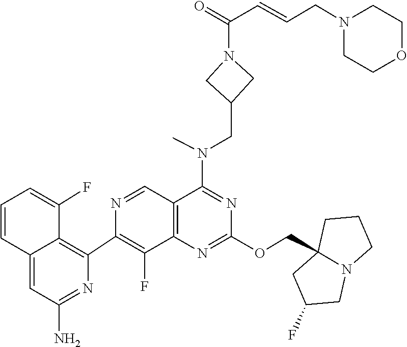 [SMILES: CN(CC1CN(C(=O)/C=C/CN2CCOCC2)C1)c1nc(OC[C@@]23CCCN2C[C@H](F)C3)nc2c(F)c(-c3nc(N)cc4cccc(F)c34)ncc12]

### Step 3: (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one

The HWE reaction was performed in a similar fashion to Example #2, Step 6. The crude residue was purified by reverse phase HPLC (column: Waters Xbridge BEH C18 100*30 mm*10 um; mobile phase: [water(NH₄HCO₃)-ACN]; B %: 20%-40%, 8 min) affording (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one (4.17 mg, 32.48%) as a yellow oil: ¹H NMR (400 MHz, CD₃CN) δ 9.30-9.20 (m, 1H), 7.54-7.43 (m, 2H), 6.90 (d, J=2.1 Hz, 1H), 6.86-6.77 (m, 1H), 6.70-6.59 (m, 1H), 6.16-6.06 (m, 1H), 5.39-5.20 (m, 1H), 5.16 (br s, 2H), 4.35-4.27 (m, 1H), 4.26-4.18 (m, 2H), 4.18-4.12 (m, 2H), 4.11-4.04 (m, 2H), 3.88-3.80 (m, 1H), 3.64-3.59 (m, 4H), 3.56 (s, 3H), 3.25-3.15 (m, 3H), 3.14-3.05 (m, 3H), 2.97-2.88 (m, 1H), 2.43-2.34 (m, 4H), 2.08-2.02 (m, 2H), 1.90-1.71 (m, 4H). LCMS Rt=2.323 min, m/z=718.3 [M+H]⁺.

LCMS (5 to 95% acetonitrile in water+0.03% ammonium bicarbonate over 6 mins) retention time 2.323 min, ESI+ found [M+H]⁺=718.3

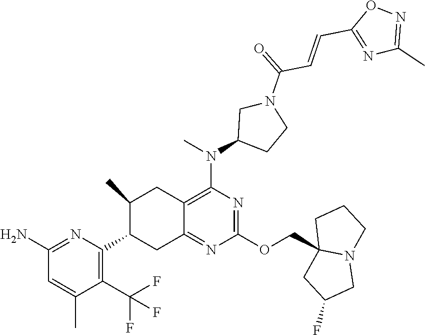 [SMILES: Cc1noc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1]

### Example 81: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

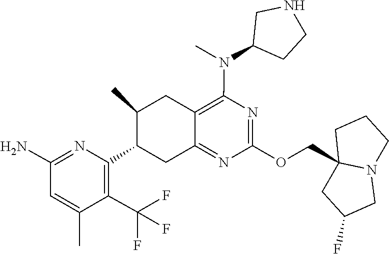 [SMILES: Cc1cc(N)nc([C@H]2Cc3nc(OC[C@@]45CCCN4C[C@H](F)C5)nc(N(C)[C@@H]4CCNC4)c3C[C@@H]2C)c1C(F)(F)F]

### Step 1: (6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-N,6-dimethyl-N—((R)-pyrrolidin-3-yl)-5,6,7,8-tetrahydroquinazolin-4-amine

The deprotection of Boc and PMB was performed in a similar fashion to Example #71, Step 7. The crude product was purified by reverse phase HPLC (column: Phenomenex Luna 80*30 mm*3 um; mobile phase: [water(TFA)-ACN]; B %: 1%-40%, 8 min) affording (6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-N,6-dimethyl-N—((R)-pyrrolidin-3-yl)-5,6,7,8-tetrahydroquinazolin-4-amine (80 mg, 88.49%, trifluoroacetic salt) as a yellow solid. LCMS Rt=1.532 min, m/z=578.3 [M+H]⁺.

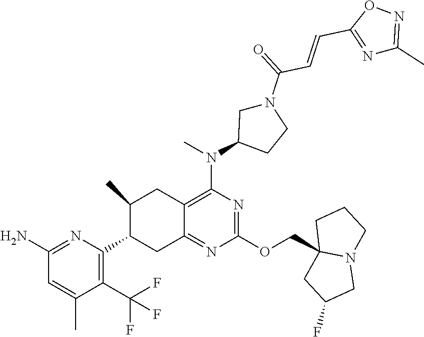 [SMILES: Cc1noc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1]

### Step 2: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one

The amide coupling reaction was performed in a similar fashion to Example #71, Step 11. The crude product was purified by reverse phase HPLC (column: Waters Xbridge BEH C18 100*30 mm*10 um; mobile phase: [water(NH₄HCO₃)-ACN]; B %: 50%-80%, 10 min) affording (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-oxadiazol-5-yl)prop-2-en-1-one (7.21 mg, 19.45%) as a yellow oil: ¹H NMR (400 MHz, CD₃CN) δ 7.50-7.30 (m, 2H), 6.28 (s, 1H), 5.34-5.15 (m, 3H), 4.72-4.57 (m, 1H), 4.06-3.92 (m, 3H), 3.88-3.67 (m, 2H), 3.64-3.40 (m, 2H), 3.22-3.10 (m, 3H), 3.08-2.98 (m, 2H), 2.94-2.83 (m, 5H), 2.67 (m, 1H), 2.52-2.44 (m, 1H), 2.41 (s, 2H), 2.39-2.37 (m, 3H), 2.32-2.27 (m, 1H), 2.16-2.06 (m, 4H), 2.02 (d, J=4.4 Hz, 1H), 1.92-1.79 (m, 3H), 0.77 (d, J=5.5 Hz, 3H). LCMS Rt=3.010 min, m/z=714.3 [M+H]⁺.

LCMS (5 to 95% acetonitrile in water+0.03% ammonium bicarbonate over 6 mins) retention time 3.010 min, ESI+ found [M+H]⁺=714.3.

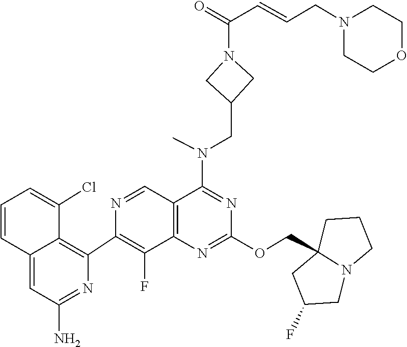 [SMILES: CN(CC1CN(C(=O)/C=C/CN2CCOCC2)C1)c1nc(OC[C@@]23CCCN2C[C@H](F)C3)nc2c(F)c(-c3nc(N)cc4cccc(Cl)c34)ncc12]

### Example 84: (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one

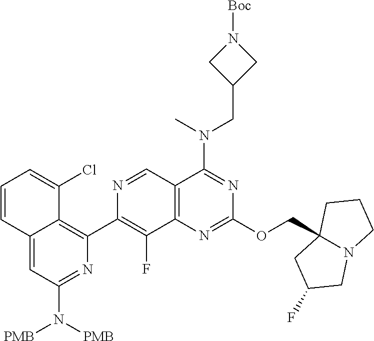

### Step 1: tert-butyl 3-(((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude residue was purified by column chromatography (silica gel, 100-200 mesh, 0-10% MeOH in DCM) affording tert-butyl 3-(((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate (550 mg, 31.26%) as a yellow oil. LCMS Rt=0.814 min, m/z=921.40 [M+H]⁺.

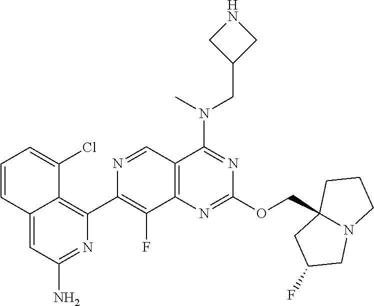 [SMILES: CN(CC1CNC1)c1nc(OC[C@@]23CCCN2C[C@H](F)C3)nc2c(F)c(-c3nc(N)cc4cccc(Cl)c34)ncc12]

### Step 2: 7-(3-amino-8-chloroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The deprotection of Boc and PMB reaction was performed in a similar fashion to Example #71, Step 7. The reaction mixture was concentrated to dryness in vacuo. The resulting residue was purified by HPLC (column: Phenomenex Luna 80*30 mm*3 um; mobile phase: [water(TFA)-ACN]; B %: 1%-30%, 8 min) affording 7-(3-amino-8-chloroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (160 mg, 37.70%, trifluoroacetic salt) as a yellow oil which was used in the next step without any further purification. LCMS Rt=0.457 min, m/z=581.2 [M+H]⁺.

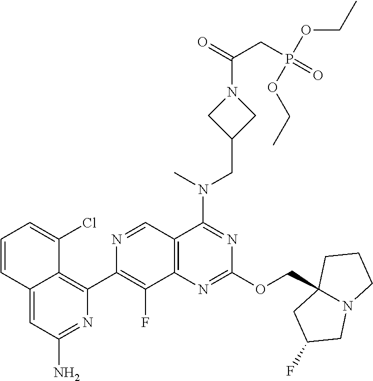 [SMILES: CCOP(=O)(CC(=O)N1CC(CN(C)c2nc(OC[C@@]34CCCN3C[C@H](F)C4)nc3c(F)c(-c4nc(N)cc5cccc(Cl)c45)ncc23)C1)OCC]

### Step 3: diethyl (2-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-2-oxoethyl)phosphonate

The amide coupling reaction was performed in a similar fashion to Example #2, Step 5. The residue was purified by HPLC (column: Waters Xbridge Prep OBD C18 150*40 mm*10 um; mobile phase: [water(NH₄HCO₃)-ACN]; B %: 25%-55%, 8 min) affording diethyl (2-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-2-oxoethyl)phosphonate (40 mg, 38.27%) as a yellow oil. LCMS Rt=1.853 min, m/z=759.3 [M+H]⁺.

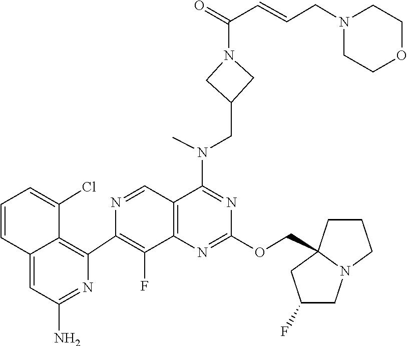 [SMILES: CN(CC1CN(C(=O)/C=C/CN2CCOCC2)C1)c1nc(OC[C@@]23CCCN2C[C@H](F)C3)nc2c(F)c(-c3nc(N)cc4cccc(Cl)c34)ncc12]

### Step 4: (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one

The HWE reaction was performed in a similar fashion to Example #2, Step 6. The residue was purified by HPLC (column: Waters Xbridge Prep OBD C18 150*40 mm*10 um; mobile phase: [water(NH₄HCO₃)-ACN]; B %: 20%-50%, 8 min) affording (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-4-morpholinobut-2-en-1-one (4.73 mg, 16.30%) as a yellow solid: ¹H NMR (400 MHz, CD₃CN) δ 9.25-9.17 (m, 1H), 7.63 (d, J=8.4 Hz, 1H), 7.42 (d, J=8.0 Hz, 1H), 7.23 (d, J=7.3 Hz, 1H), 6.92 (s, 1H), 6.70-6.57 (m, 1H), 6.10 (d, J=15.3 Hz, 1H), 5.34-5.15 (m, 3H), 4.31 (t, J=8.4 Hz, 1H), 4.19-4.06 (m, 6H), 3.82 (m, 1H), 3.61 (s, 3H), 3.55 (s, 3H), 3.14 (s, 3H), 3.07 (d, J=6.4 Hz, 3H), 2.88 (d, J=6.9 Hz, 1H), 2.38 (m, 5H), 2.09-2.04 (m, 3H), 1.91-1.74 (m, 3H). LCMS Rt=2.378 min, m/z=734.3 [M+H]⁺.

LCMS (5 to 95% acetonitrile in water+0.03% ammonium bicarbonate over 6 mins) retention time 2.378 min, ESI+ found [M+H]⁺=734.3.

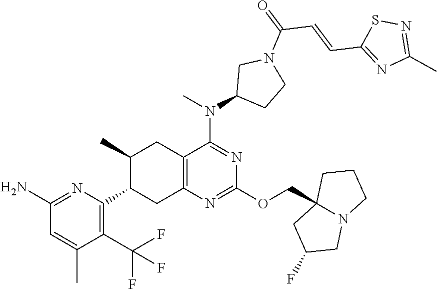 [SMILES: Cc1nsc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1]

### Example 88: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one

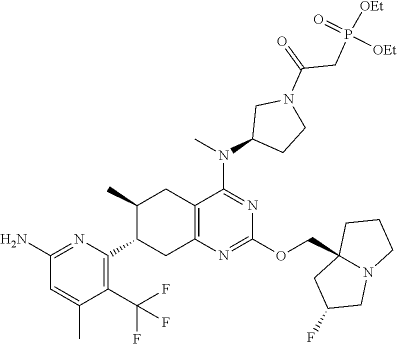 [SMILES: CCOP(=O)(CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1)OCC]

### Step 1: diethyl (2-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-2-oxoethyl)phosphonate

The amide coupling reaction was performed in a similar fashion to Example #2, Step 5. The crude product was purified by reverse phase HPLC (column: Waters Xbridge Prep OBD C18 150*40 mm*10 um; mobile phase: [water(NH₄HCO₃)-ACN]; B %: 30%-60%, 8 min) affording diethyl (2-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-2-oxoethyl)phosphonate (25 mg, 22.88%) as a white solid.

LCMS Rt=0.857 min, m/z=756.4 [M+H]⁺.

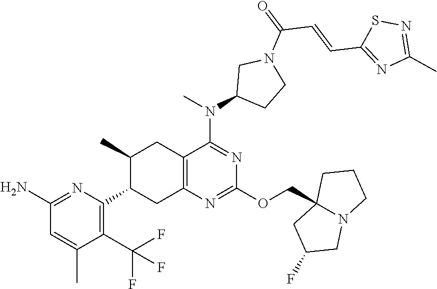 [SMILES: Cc1nsc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1]

### Step 2: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one

To a solution of diethyl (2-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-2-oxoethyl)phosphonate (20 mg, 26.46 umol) in MeCN (2 mL) was added N-ethyl-N-isopropyl-propan-2-amine (20.52 mg, 158.78 umol) and LiCl (11.22 mg, 264.63 umol), the mixture was degassed and purged with nitrogen for 3 times and then 3-methyl-1,2,4-thiadiazole-5-carbaldehyde (10.17 mg, 79.39 umol) was added to the mixture. The mixture was stirred at 25° C. for 1 h under nitrogen atmosphere. The crude product was purified by reverse phase prep-HPLC (column: Waters Xbridge Prep OBD C18 150*40 mm*10 um; mobile phase: [water(NH₄HCO₃)-ACN]; B %: 40%-60%, 8 min) affording (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one (11.23 mg, 58.15%) as a yellow amorphous solid: ¹H NMR (400 MHz, CD₃CN) δ 7.71 (dd, J=7.2, 15.3 Hz, 1H), 7.36-7.22 (m, 1H), 6.26 (s, 1H), 5.40-5.09 (m, 3H), 4.69-4.56 (m, 1H), 4.06-3.91 (m, 3H), 3.88-3.77 (m, 1H), 3.76-3.66 (m, 1H), 3.64-3.54 (m, 1H), 3.52-3.40 (m, 1H), 3.22-3.09 (m, 3H), 3.08-2.99 (m, 2H), 2.91 (d, J=1.8 Hz, 3H), 2.88-2.80 (m, 2H), 2.64 (d, J=10.6 Hz, 3H), 2.61-2.53 (m, 1H), 2.50-2.39 (m, 2H), 2.39-2.34 (m, 3H), 2.32-2.18 (m, 2H), 2.06-1.99 (m, 2H), 1.91-1.72 (m, 3H), 0.75 (d, J=6.4 Hz, 3H). LCMS Rt=3.051 min, m/z=730.3 [M+H]⁺.

LCMS (5 to 95% acetonitrile in water+0.03% ammonium bicarbonate over 6 mins) retention time 3.051 min, ESI+ found [M+H]⁺=730.3.

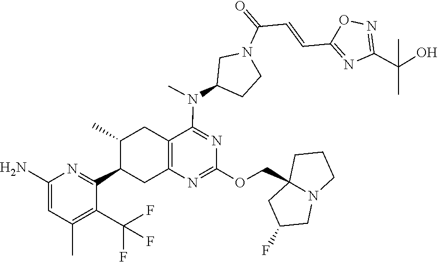 [SMILES: Cc1cc(N)nc([C@@H]2Cc3nc(OC[C@@]45CCCN4C[C@H](F)C5)nc(N(C)[C@@H]4CCN(C(=O)/C=C/c5nc(C(C)(C)O)no5)C4)c3C[C@H]2C)c1C(F)(F)F]

### Example 89: (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one

### Step 1: (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one

The amide coupling reaction was performed in a similar fashion to Example #71, Step 11. The crude product was purified by reverse phase prep-HPLC (column: Waters Xbridge BEH C18 100*30 mm*10 um; mobile phase: [water(NH₄HCO₃)-ACN]; B %: 20%-60%, 8 min) affording (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one (10.01 mg, 18.08%) as a white solid: ¹H NMR (400 MHz, CD₃CN) δ 7.53-7.46 (m, 1H), 7.43-7.36 (m, 1H), 6.28 (s, 1H), 5.34-5.14 (m, 3H), 4.80-4.61 (m, 1H), 4.25-4.00 (m, 2H), 3.99-3.86 (m, 2H), 3.85-3.65 (m, 2H), 3.52-3.39 (m, 1H), 3.23-3.08 (m, 3H), 3.06-2.98 (m, 2H), 2.94 (d, J=4.5 Hz, 3H), 2.90-2.81 (m, 2H), 2.68-2.61 (m, 1H), 2.56-2.48 (m, 1H), 2.41-2.36 (m, 3H), 2.33-2.25 (m, 1H), 2.18-2.13 (m, 2H), 2.09-2.05 (m, 2H), 2.04-2.00 (m, 1H), 1.91-1.75 (m, 3H), 1.61 (d, J=5.1 Hz, 6H), 0.77 (d, J=6.2 Hz, 3H). LCMS Rt=2.864 min, m/z=758.4 [M+H]⁺.

LCMS (5 to 95% acetonitrile in water+0.03% ammonium bicarbonate over 6 mins) retention time 2.864 min, ESI+ found [M+H]⁺=758.4.

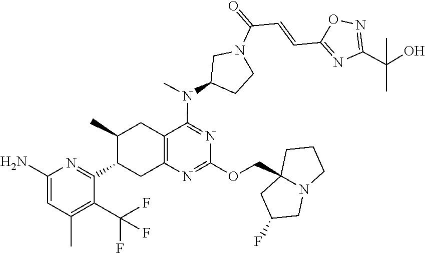 [SMILES: Cc1cc(N)nc([C@H]2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F]

### Example 94: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one

### Step 1: (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one

The amide coupling reaction was performed in a similar fashion to Example #71, Step 11. The crude product was purified by reverse phase prep-HPLC (column: Waters Xbridge BEH C18 100*30 mm*10 um; mobile phase: [water(NH₄HCO₃)-ACN]; B %: 35%-65%, 8 min) affording (E)-1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-(2-hydroxypropan-2-yl)-1,2,4-oxadiazol-5-yl)prop-2-en-1-one (19.27 mg, 29.31%) as a yellow solid: ¹H NMR (400 MHz, CD₃CN) δ 7.54-7.28 (m, 2H), 6.28 (s, 1H), 5.38-5.12 (m, 3H), 4.74-4.49 (m, 1H), 4.08-3.91 (m, 3H), 3.88-3.78 (m, 1H), 3.73 (d, J=8.1 Hz, 1H), 3.66-3.56 (m, 1H), 3.54-3.41 (m, 1H), 3.24-3.08 (m, 3H), 3.07-2.97 (m, 2H), 2.92 (s, 3H), 2.89-2.81 (m, 2H), 2.68 (d, J=14.6 Hz, 1H), 2.52-2.42 (m, 1H), 2.38 (s, 3H), 2.07 (s, 3H), 1.94-1.74 (m, 6H), 1.60 (d, J=6.4 Hz, 6H), 0.83-0.73 (m, 3H). LCMS Rt=2.928 min, m/z=758.4 [M+H]⁺.

LCMS (5 to 95% acetonitrile in water+0.03% ammonium bicarbonate over 6 mins) retention time 2.928 min, ESI+ found [M+H]⁺=758.4.

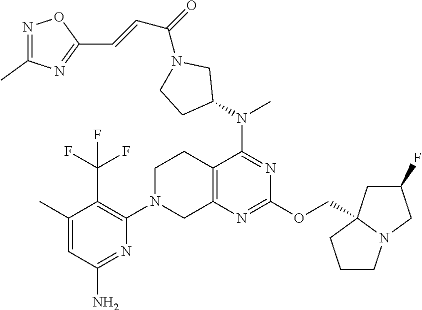 [SMILES: Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1]

### Example 95: (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-methyl-1,2,4-oxadiazol-5-yl)prop-2-en-1-one

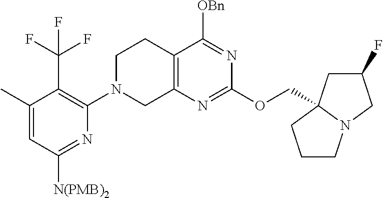

### Step 1: 6-(4-(benzyloxy)-2-(((2R,7aS)-2-fluorotetrahydro-1H-pyrrolizin-7a(5H)-yl)methoxy)-5,8-dihydropyrido[3,4-d]pyrimidin-7(6H)-yl)-N,N-bis(4-methoxybenzyl)-4-methyl-5-(trifluoromethyl)pyridin-2-amine

To a solution of 6-bromo-N,N-bis(4-methoxybenzyl)-4-methyl-5-(trifluoromethyl)pyridin-2-amine (1.66 g, 3.35 mmol) and 4-(benzyloxy)-2-(((2R,7aS)-2-fluorotetrahydro-1H-pyrrolizin-7a(5H)-yl)methoxy)-5,6,7,8-tetrahydropyrido[3,4-d]pyrimidine (2 g, 5.02 mmol) in 1,4-dioxane (15 mL) was added Cs₂CO₃ (3.27 g, 10.04 mmol), Pd₂(dba)₃ (306.41 mg, 334.61 umol) and RuPhos (156.14 mg, 334.61 umol). The resulting mixture was stirred at 100° C. for 12 h under nitrogen atmosphere. The reaction mixture was quenched with water (10 mL) and extracted with EtOAc (3×10 mL). The combined organic layers were dried over sodium sulfate and concentrated in vacuo. The crude residue was purified by column chromatography (silica gel, 100-200 mesh, 0-100% EtOAc in petroleum ether) affording 6-(4-(benzyloxy)-2-(((2R,7aS)-2-fluorotetrahydro-1H-pyrrolizin-7a(5H)-yl)methoxy)-5,8-dihydropyrido[3,4-d]pyrimidin-7(6H)-yl)-N,N-bis(4-methoxybenzyl)-4-methyl-5-(trifluoromethyl)pyridin-2-amine (1.5 g, 44.12%) as a yellow gum. LCMS Rt=0.962 min, m/z=813.4 [M+H]⁺.

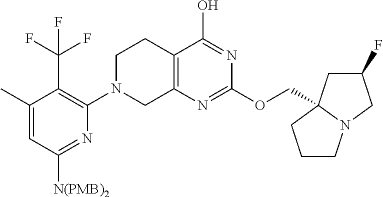

### Step 2: 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol

To a solution of 6-(4-(benzyloxy)-2-(((2R,7aS)-2-fluorotetrahydro-1H-pyrrolizin-7a(5H)-yl)methoxy)-5,8-dihydropyrido[3,4-d]pyrimidin-7(6H)-yl)-N,N-bis(4-methoxybenzyl)-4-methyl-5-(trifluoromethyl)pyridin-2-amine (1.1 g, 1.35 mmol) in EtOAc (1 mL) was added Pd/C (100 mg, 1.35 mmol, 10 wt. % loading) and the mixture was stirred at 20° C. for 1 h under hydrogen (15 Psi). The reaction mixture was filtered though Celite®, the filtrate was concentrated in vacuo affording 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (780 mg, crude) as a yellow solid used in next step without further purification. LCMS Rt=0.790 min, m/z=723.3 [M+H]⁺.

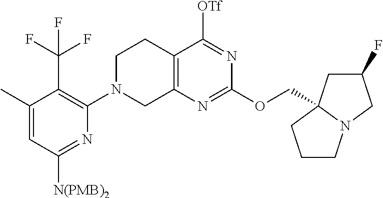

### Step 3: 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl trifluoromethanesulfonate

To a solution of 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol in DCM (100 mL) was added NEt₃ and trifluoromethanesulfonic anhydride at 0° C. under nitrogen atmosphere and the resulting mixture was stirred for 1 h at 20° C. under nitrogen atmosphere. The reaction mixture was then quenched with sat. NaHCO₃ (30 mL) at 0° C. and extracted with DCM (3×100 mL). The reaction mixture was concentrated in vacuo affording 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl trifluoromethanesulfonate (700 mg, crude) as a brown gum used in next step without further purification. LCMS Rt=2.673 min, m/z=855.3 [M+H]⁺.

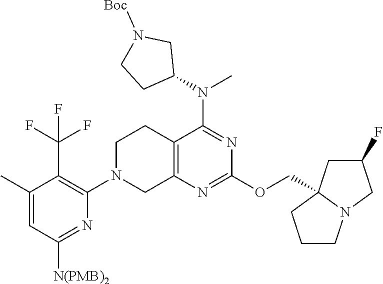

### Step 4: tert-butyl (R)-3-((7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidine-1-carboxylate

To a solution of tert-butyl (R)-3-(methylamino)pyrrolidine-1-carboxylate in DMF (5 mL) was added DIPEA, 4 Å molecular sieve and 7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl trifluoromethanesulfonate at 0° C. The resulting mixture was stirred at 20° C. for 12 h. The reaction mixture was then filtered and concentrated to dryness in vacuo. The crude residue was purified by column chromatography (silica gel, 100-200 mesh, 0-100% EtOAc in petroleum ether) affording tert-butyl (R)-3-((7-(6-(bis(4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidine-1-carboxylate (700 mg, 66.12%) as a yellow gum. LCMS Rt=0.795 min, m/z=905.5 [M+H]⁺.

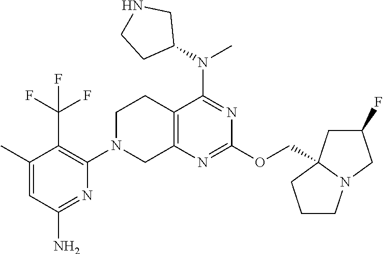 [SMILES: Cc1cc(N)nc(N2CCc3c(nc(OC[C@@]45CCCN4C[C@H](F)C5)nc3N(C)[C@@H]3CCNC3)C2)c1C(F)(F)F]

### Step 5: 7-(6-amino-4-methyl-3-(trifluoromethyl)pyridin-2-yl)-2-(((2R,7aS)-2-fluorotetrahydro-1H-pyrrolizin-7a(5H)-yl)methoxy)-N-methyl-N—((R)-pyrrolidin-3-yl)-5,6,7,8-tetrahydropyrido[3,4-d]pyrimidin-4-amine

The deprotection of Boc and PMB reaction was performed in a similar fashion to Example #71, Step 7. The reaction mixture was concentrated in vacuo affording 7-(6-amino-4-methyl-3-(trifluoromethyl)pyridin-2-yl)-2-(((2R,7aS)-2-fluorotetrahydro-1H-pyrrolizin-7a(5H)-yl)methoxy)-N-methyl-N—((R)-pyrrolidin-3-yl)-5,6,7,8-tetrahydropyrido[3,4-d]pyrimidin-4-amine (500 mg, crude, trifluoroacetic salt) as a brown oil, used in next step without further purification. LCMS Rt=0.474 min, m/z=565.3 [M+H]⁺.

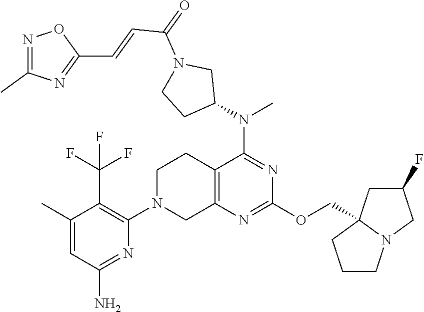 [SMILES: Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1]

### Step 6: (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-methyl-1,2,4-oxadiazol-5-yl)prop-2-en-1-one

The amide coupling reaction was performed in a similar fashion to Example #71, Step 11. The crude product was purified by reverse phase prep-HPLC (column: Waters Xbridge Prep OBD C18 150*40 mm*10 um; mobile phase: [water (NH₄HCO₃)-ACN]; B %: 40%-60%, 8 min) affording (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-methyl-1,2,4-oxadiazol-5-yl)prop-2-en-1-one (20.72 mg, 24.71%) as a white solid: ¹H NMR (400 MHz, CD₃CN) δ 7.51-7.25 (m, 2H), 6.03 (s, 1H), 5.29 (m, 1H), 5.15 (br s, 2H), 4.80-4.71 (m, 1H), 4.21 (s, 2H), 4.07-3.99 (m, 2H), 3.94 (s, 1H), 3.81-3.73 (m, 1H), 3.65 (dd, J=9.8, 17.8 Hz, 1H), 3.45-3.37 (m, 2H), 3.32-3.25 (m, 1H), 3.10 (m, 2H), 3.02 (m, 1H), 2.93 (s, 3H), 2.85 (dd, J=7.3, 14.8 Hz, 2H), 2.74 (d, J=4.5 Hz, 1H), 2.38 (d, J=6.0 Hz, 3H), 2.30 (s, 3H), 2.13-2.10 (m, 2H), 2.05 (s, 3H), 1.91-1.73 (m, 3H). LCMS Rt=3.081 min, m/z=701.3 [M+H]⁺.

LCMS (5 to 95% acetonitrile in water+0.03% ammonium bicarbonate over 6 mins) retention time 3.081 min, ESI+ found [M+H]⁺=701.3.

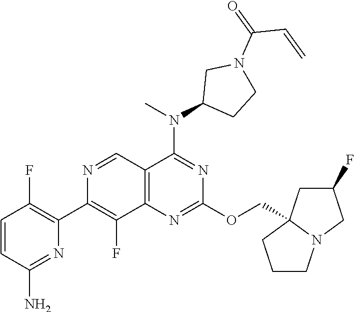 [SMILES: C=CC(=O)N1CC[C@@H](N(C)c2nc(OC[C@@]34CCCN3C[C@H](F)C4)nc3c(F)c(-c4nc(N)ccc4F)ncc23)C1]

### Example 96: 1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

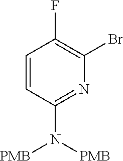

### Step 1: 6-bromo-5-fluoro-N,N-bis[(4-methoxyphenyl)methyl]pyridin-2-amine

To a solution of 6-bromo-5-fluoro-pyridin-2-amine (750 mg, 3.93 mmol) in DMF (20 mL) was added NaH (785.26 mg, 19.63 mmol, 60% dispersion in mineral oil) at 0° C. and the resulting mixture was stirred for 30 min at this temperature. Then, 4-methoxybenzyl chloride (1.54 g, 9.82 mmol) was added and the solution was warmed to 25° C. and stirring was continued for 1 h. After this time, the reaction mixture was quenched with water (100 mL) and the resulting precipitate was collected by filtration to afford 6-bromo-5-fluoro-N,N-bis[(4-methoxyphenyl)methyl]pyridin-2-amine (1.2 g, crude) as a brown solid which was used in the next step without any further purification. LCMS Rt=3.090 min, m/z=431.1 [M+H]⁺.

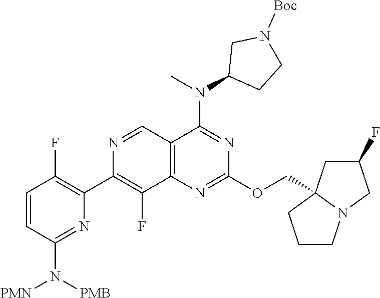

### Step 2: tert-butyl (R)-3-((7-(6-(bis(4-methoxybenzyl)amino)-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The crude residue was purified by column chromatography (silica gel, 100-200 mesh, 0-100% EtOAc in petroleum ether) affording tert-butyl (R)-3-((7-(6-(bis(4-methoxybenzyl)amino)-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (440 mg, 514.65 umol) as a brown oil. LCMS Rt=0.977 min, m/z=855.4 [M+H]⁺.

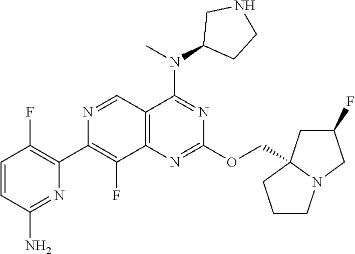 [SMILES: CN(c1nc(OC[C@@]23CCCN2C[C@H](F)C3)nc2c(F)c(-c3nc(N)ccc3F)ncc12)[C@@H]1CCNC1]

### Step 3: 7-(6-amino-3-fluoropyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB reaction was performed in a similar fashion to Example #71, Step 7. The crude product was purified by reverse phase HPLC (column: Waters Xbridge Prep OBD C18 150*40 mm*10 um; mobile phase: [water(NH₄HCO₃)-ACN]; B %: 5%-40%, 8 min) to afford 7-(6-amino-3-fluoropyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (80 mg, 33.23%) as brown oil. LCMS Rt=0.588 min, m/z=515.2 [M+H]⁺.

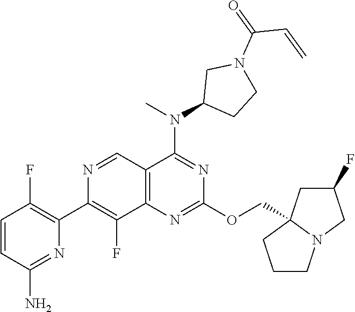 [SMILES: C=CC(=O)N1CC[C@@H](N(C)c2nc(OC[C@@]34CCCN3C[C@H](F)C4)nc3c(F)c(-c4nc(N)ccc4F)ncc23)C1]

### Step 4: 1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The acylation reaction was performed in a similar fashion to Example #71, Step 11. The crude product was purified by reverse phase HPLC(column: Waters Xbridge BEH C18 100*30 mm*10 um; mobile phase: [water(NH₄HCO₃)-ACN]; B %: 25%-55%, 10 min) affording 1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (4.39 mg, 12.27%) as a yellow solid. ¹H NMR (400 MHz, CD₃CN) δ 9.23 (s, 1H), 7.43 (t, J=9.1 Hz, 1H), 6.68 (dd, J=2.9, 8.9 Hz, 1H), 6.63-6.52 (m, 1H), 6.30-6.20 (m, 1H), 5.75-5.64 (m, 1H), 5.42-5.19 (m, 2H), 5.03 (br s, 2H), 4.27-4.21 (m, 1H), 4.19-4.12 (m, 1H), 4.10-3.78 (m, 2H), 3.70-3.62 (m, 1H), 3.59-3.43 (m, 1H), 3.40 (s, 3H), 3.20-3.13 (m, 2H), 3.09 (s, 1H), 2.97-2.88 (m, 1H), 2.41-2.35 (m, 1H), 2.33-2.25 (m, 1H), 2.13 (d, J=2.6 Hz, 1H), 2.10-2.03 (m, 1H), 1.95-1.81 (m, 4H). LCMS Rt=2.410 min, m/z=569.3 [M+H]⁺.

LCMS (5 to 95% acetonitrile in water+0.03% ammonium bicarbonate over 6 mins) retention time 2.410 min, ESI+ found [M+H]⁺=569.3.

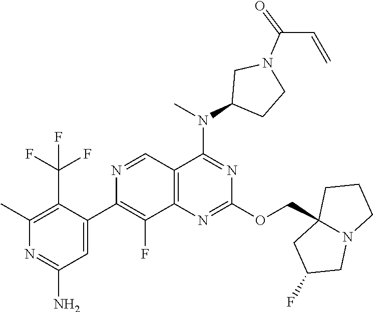 [SMILES: C=CC(=O)N1CC[C@@H](N(C)c2nc(OC[C@@]34CCCN3C[C@H](F)C4)nc3c(F)c(-c4cc(N)nc(C)c4C(F)(F)F)ncc23)C1]

### Example 107: 1-((R)-3-((7-(6-amino-2-methyl-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

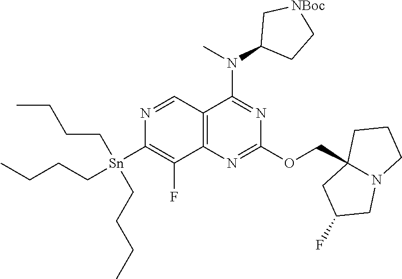 [SMILES: CCC[CH2][Sn]([CH2]CCC)([CH2]CCC)[c]1ncc2c(N(C)[C@@H]3CCN(C(=O)OC(C)(C)C)C3)nc(OC[C@@]34CCCN3C[C@H](F)C4)nc2c1F]

### Step 1: tert-butyl (R)-3-((8-fluoro-2-(((2R,7aS)-2-fluorotetrahydro-1H-pyrrolizin-7a(5H)-yl)methoxy)-7-(tributylstannyl)pyrido[4,3-d]pyrimidin-4-yl)(methyl)amino)pyrrolidine-1-carboxylate

The tin reagent reaction was performed in a similar fashion to Example #71, Step 5. The reaction mixture was concentrated in vacuo and purified by column chromatography (silica gel, 100-200 mesh, 2%-100% EtOAc in petroleum ether) affording tert-butyl (R)-3-((8-fluoro-2-(((2R,7aS)-2-fluorotetrahydro-1H-pyrrolizin-7a(5H)-yl)methoxy)-7-(tributylstannyl)pyrido[4,3-d]pyrimidin-4-yl)(methyl)amino)pyrrolidine-1-carboxylate (1.2 g, 40.75%) as a yellow oil. LCMS Rt=0.778 min, m/z=795.4 [M+H]⁺.

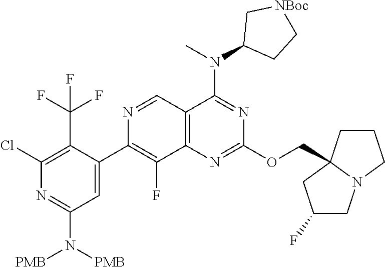

### Step 2: tert-butyl (R)-3-((7-(6-(bis(4-methoxybenzyl)amino)-2-chloro-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The reaction mixture was concentrated in vacuo and purified by reverse phase HPLC (column: Phenomenex Luna C18 250*50 mm*10 um; mobile phase: [water(TFA)-ACN]; B %: 50%-90%, 10 min) affording tert-butyl (R)-3-((7-(6-(bis(4-methoxybenzyl)amino)-2-chloro-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (200 mg, 15.13%, trifluoroacetic salt) as a white solid. LCMS Rt=0.913 min, m/z=939.4 [M+H]⁺.

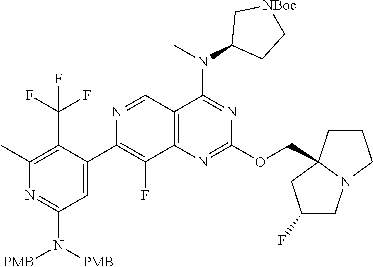

### Step 3: tert-butyl (R)-3-((7-(6-(bis(4-methoxybenzyl)amino)-2-methyl-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

A mixture of tert-butyl (R)-3-((7-(6-(bis(4-methoxybenzyl)amino)-2-chloro-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (200 mg, 189.86 umol, trifluoroacetic salt), methylboronic acid (34.09 mg, 569.57 umol), Na₂CO₃ (60.37 mg, 569.57 umol) and Pd(PPh₃)₄ (21.94 mg, 18.99 umol) in 1,4-dioxane (6 mL) and water (2 mL) was evacuated and backfilled with nitrogen 3 times. The resulting mixture was then stirred at 100° C. for 12 h under nitrogen atmosphere. After this time, the mixture was cooled to room temperature, diluted with water (20 mL) and extracted with EtOAc (3×50 mL). The combined organic layers were dried over sodium sulfate and concentrated in vacuo affording tert-butyl (R)-3-((7-(6-(bis(4-methoxybenzyl)amino)-2-methyl-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (180 mg, crude) as a yellow oil used in next step without further purification. LCMS Rt=0.832 min, m/z=919.4 [M+H]⁺.

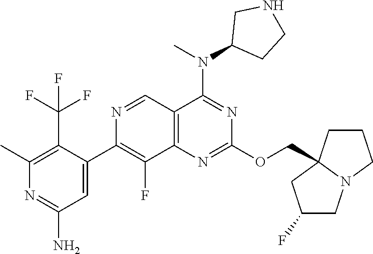 [SMILES: Cc1nc(N)cc(-c2ncc3c(N(C)[C@@H]4CCNC4)nc(OC[C@@]45CCCN4C[C@H](F)C5)nc3c2F)c1C(F)(F)F]

### Step 4: 7-(6-amino-2-methyl-3-(trifluoromethyl)pyridin-4-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB was performed in a similar fashion to Example #71, Step 7. The reaction mixture was concentrated in vacuo affording 7-(6-amino-2-methyl-3-(trifluoromethyl)pyridin-4-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (180 mg, crude, trifluoroacetic salt) as a yellow oil used in next step without further purification. LCMS Rt=0.684 min, m/z=579.3 [M+H]⁺.

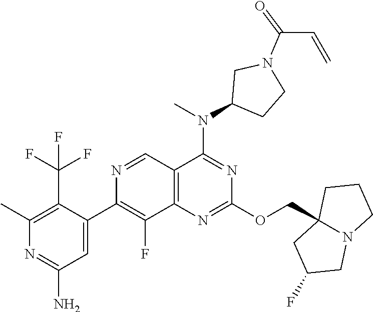 [SMILES: C=CC(=O)N1CC[C@@H](N(C)c2nc(OC[C@@]34CCCN3C[C@H](F)C4)nc3c(F)c(-c4cc(N)nc(C)c4C(F)(F)F)ncc23)C1]

### Step 5: 1-((R)-3-((7-(6-amino-2-methyl-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The acylation reaction was performed in a similar fashion to Example #71, Step 11. The reaction mixture was concentrated in vacuo and purified by reverse phase HPLC (column: Phenomenex Luna C18 200*40 mm*10 um; mobile phase: [water(FA)-ACN]; B %: 1%-30%, 8 min) affording 1-((R)-3-((7-(6-amino-2-methyl-3-(trifluoromethyl)pyridin-4-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (14.2 mg, 7.98%, formic acid) as a white solid: ¹H NMR (400 MHz, CD₃CN) δ 9.14 (d, J=1.3 Hz, 1H), 6.57 (dt, J=10.3, 16.4 Hz, 1H), 6.34-6.16 (m, 2H), 5.67 (ddd, J=2.1, 7.5, 10.1 Hz, 1H), 5.51-5.18 (m, 4H), 4.27-4.13 (m, 2H), 4.09-3.77 (m, 2H), 3.69-3.47 (m, 2H), 3.39 (d, J=1.1 Hz, 3H), 3.21 (s, 1H), 3.12 (br s, 2H), 2.95-2.90 (m, 1H), 2.54 (d, J=2.3 Hz, 3H), 2.39-2.25 (m, 2H), 2.22-2.01 (m, 3H), 1.93-1.77 (m, 3H). LCMS Rt=1.801 min, m/z=633.3 [M+H]⁺.

LCMS (5 to 95% acetonitrile in water+0.1% TFA over 6 mins) retention time 1.801 min, ESI+ found [M+H]⁺=633.3.

### Example 111: 1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)prop-2-en-1-one

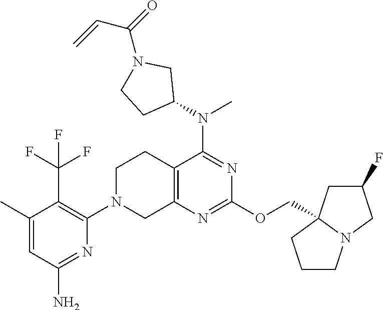 [SMILES: C=CC(=O)N1CC[C@@H](N(C)c2nc(OC[C@@]34CCCN3C[C@H](F)C4)nc3c2CCN(c2nc(N)cc(C)c2C(F)(F)F)C3)C1]

### Step 1: 1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)prop-2-en-1-one

The coupling reaction was performed in a similar fashion to Example #71, Step 11. The reaction mixture was purified by reverse phase HPLC (column: Waters Xbridge Prep OBD C18 150*40 mm*10 um; mobile phase: [water(NH₄HCO₃)-ACN]; B %: 40%-60%, 8 min) affording 1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)prop-2-en-1-one (6.28 mg, 5.52%) as a white solid: ¹H NMR (400 MHz, CD₃CN) δ 6.62-6.44 (m, 1H), 6.27-6.13 (m, 1H), 6.01 (s, 1H), 5.62 (ddd, J=2.4, 7.6, 10.1 Hz, 1H), 5.31-5.25 (m, 1H), 5.19-5.11 (m, 3H), 4.80-4.67 (m, 1H), 4.19 (s, 2H), 4.01-3.98 (m, 1H), 3.94-3.88 (m, 2H), 3.83-3.76 (m, 1H), 3.59-3.49 (m, 1H), 3.40-3.34 (m, 2H), 3.26 (d, J=1.8 Hz, 1H), 3.09 (s, 2H), 3.03-3.00 (m, 1H), 2.91 (d, J=2.8 Hz, 3H), 2.87-2.81 (m, 2H), 2.73-2.69 (m, 1H), 2.28 (d, J=2.0 Hz, 3H), 2.18 (s, 1H), 2.04 (d, J=3.1 Hz, 2H), 1.81 (dd, J=7.5, 13.4 Hz, 4H). LCMS Rt=2.941 min, m/z=619.3 [M+H]⁺.

LCMS (5 to 95% acetonitrile in water+0.03% ammonium bicarbonate over 6 mins) retention time 2.941 min, ESI+ found [M+H]⁺=619.3.

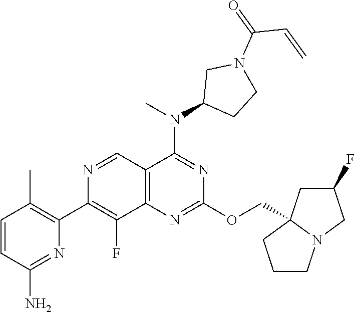 [SMILES: C=CC(=O)N1CC[C@@H](N(C)c2nc(OC[C@@]34CCCN3C[C@H](F)C4)nc3c(F)c(-c4nc(N)ccc4C)ncc23)C1]

### Example 125: 1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

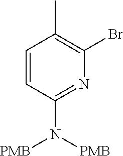

### Step 1: 6-bromo-N,N-bis(4-methoxybenzyl)-5-methylpyridin-2-amine

To a solution of 6-bromo-5-methyl-pyridin-2-amine (1.9 g, 10.16 mmol) in DMF (15 mL) was added NaH (2.03 g, 50.79 mmol, 60% dispersion in mineral oil) and 4-methoxybenzyl chloride (3.98 g, 25.40 mmol) at 0° C. The resulting mixture was allowed to warm slowly to room temperature and stirring was continued for 2 h. The reaction mixture was then concentrated in vacuo and purified by column chromatography (silica gel, 100-200 mesh, 60-80% EtOAc in petroleum ether) affording 6-bromo-N,N-bis(4-methoxybenzyl)-5-methylpyridin-2-amine (3 g, 69.11%) as a white solid. LCMS Rt=0.967 min, m/z=427.1 [M+H]⁺.

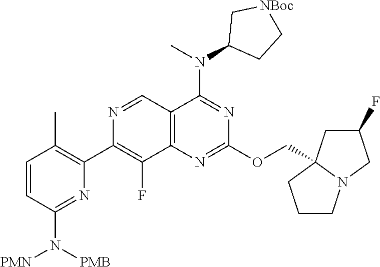

### Step 2: tert-butyl (R)-3-((7-(6-(bis(4-methoxybenzyl)amino)-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The reaction mixture was concentrated in vacuo and purified by reverse phase HPLC (column: Phenomenex Luna C18 250*50 mm*10 um; mobile phase: [water(TFA)-ACN]; B %: 30%-70%, 10 min) affording tert-butyl (R)-3-((7-(6-(bis(4-methoxybenzyl)amino)-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (360 mg, 59.21%, trifluoroacetic salt) as a yellow oil. LCMS Rt=0.825 min, m/z=851.4 [M+H]⁺.

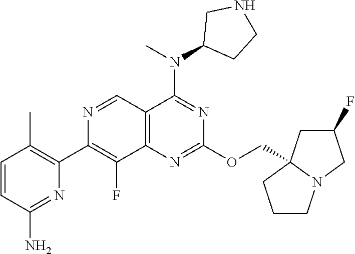 [SMILES: Cc1ccc(N)nc1-c1ncc2c(N(C)[C@@H]3CCNC3)nc(OC[C@@]34CCCN3C[C@H](F)C4)nc2c1F]

### Step 3: 7-(6-amino-3-methylpyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB reaction was performed in a similar fashion to Example #71, Step 7. The reaction mixture was concentrated in vacuo affording 7-(6-amino-3-methylpyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (75 mg, crude, trifluoroacetic salt) as a yellow oil used in next step without any further purification. LCMS Rt=0.363 min, m/z=511.3 [M+H]⁺.

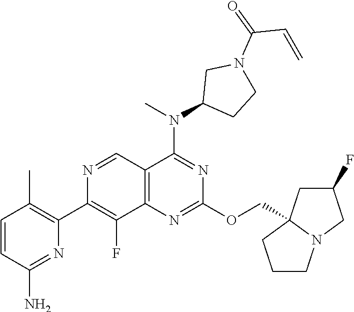 [SMILES: C=CC(=O)N1CC[C@@H](N(C)c2nc(OC[C@@]34CCCN3C[C@H](F)C4)nc3c(F)c(-c4nc(N)ccc4C)ncc23)C1]

### Step 4: 1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The acylation reaction was performed in a similar fashion to Example #71, Step 11. The reaction mixture was concentrated in vacuo and purified by reverse phase HPLC (column: Waters Xbridge BEH C18 100*30 mm*10 um; mobile phase: [water(NH₄HCO₃)-ACN]; B %: 15%-45%, 8 min) affording 1-((R)-3-((7-(6-amino-3-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (17.62 mg, 20.59%) as a white solid: ¹H NMR (400 MHz, CD₃CN) δ 9.26-9.15 (m, 1H), 7.45 (d, J=8.3 Hz, 1H), 6.69-6.54 (m, 2H), 6.34-6.21 (m, 1H), 5.71 (ddd, J=2.3, 6.8, 10.2 Hz, 1H), 5.45-5.35 (m, 1H), 5.35-5.21 (m, 1H), 4.87 (s, 2H), 4.26-4.20 (m, 1H), 4.17-4.12 (m, 1H), 4.01-3.79 (m, 2H), 3.72-3.63 (m, 1H), 3.61-3.45 (m, 1H), 3.42 (s, 3H), 3.19-3.14 (m, 2H), 3.10 (s, 1H), 2.97-2.90 (m, 1H), 2.45-2.37 (m, 1H), 2.34-2.28 (m, 1H), 2.14 (d, J=2.8 Hz, 2H), 2.09-2.07 (m, 3H), 1.97-1.81 (m, 4H). LCMS Rt=2.455 min, m/z=565.3 [M+H]⁺.

LCMS (5 to 95% acetonitrile in water+0.03% ammonium bicarbonate over 6 mins) retention time 2.455 min, ESI+ found [M+H]⁺=565.3.

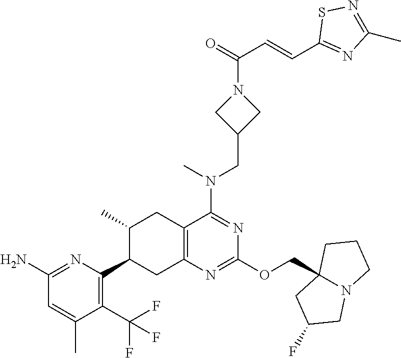 [SMILES: Cc1nsc(/C=C/C(=O)N2CC(CN(C)c3nc(OC[C@@]45CCCN4C[C@H](F)C5)nc4c3C[C@@H](C)[C@H](c3nc(N)cc(C)c3C(F)(F)F)C4)C2)n1]

### Example 126: (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one

### Step 1: (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one

The amide coupling reaction was performed in a similar fashion to Example #71, Step 11. The reaction mixture was purified by reverse phase HPLC (column: Waters Xbridge BEH C18 100*30 mm*10 um; mobile phase: [water(NH₄HCO₃)-ACN]; B %: 30%-70%, 8 min) affording (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one (13.77 mg, 14.22%) as a white solid: ¹H NMR (400 MHz, CD₃CN) δ 7.64 (dd, J=3.8, 15.4 Hz, 1H), 7.00 (dd, J=8.1, 15.5 Hz, 1H), 6.26 (s, 1H), 5.39-5.01 (m, 3H), 4.37 (q, J=8.5 Hz, 1H), 4.19-4.03 (m, 2H), 4.03-3.95 (m, 2H), 3.94-3.88 (m, 1H), 3.84-3.64 (m, 1H), 3.53-3.38 (m, 1H), 3.20-3.13 (m, 1H), 3.10 (d, J=2.8 Hz, 3H), 3.03 (d, J=3.3 Hz, 4H), 2.96 (d, J=10.9 Hz, 1H), 2.90-2.76 (m, 2H), 2.69-2.60 (m, 4H), 2.47 (dd, J=11.3, 15.6 Hz, 1H), 2.36 (q, J=3.5 Hz, 3H), 2.14-2.06 (m, 2H), 2.05-1.98 (m, 2H), 1.91-1.73 (m, 3H), 0.75 (d, J=6.4 Hz, 3H). LCMS Rt=2.990 min, m/z=730.3 [M+H]⁺.

LCMS (5 to 95% acetonitrile in water+0.03% ammonium bicarbonate over 6 mins) retention time 2.990 min, ESI+ found [M+H]⁺=730.3.

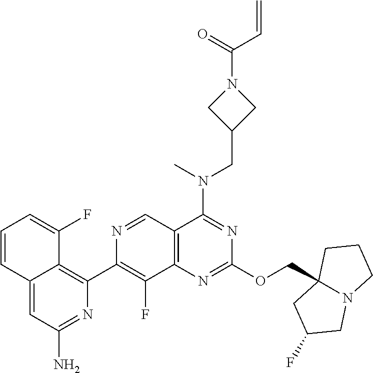 [SMILES: C=CC(=O)N1CC(CN(C)c2nc(OC[C@@]34CCCN3C[C@H](F)C4)nc3c(F)c(-c4nc(N)cc5cccc(F)c45)ncc23)C1]

### Example 128: 1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

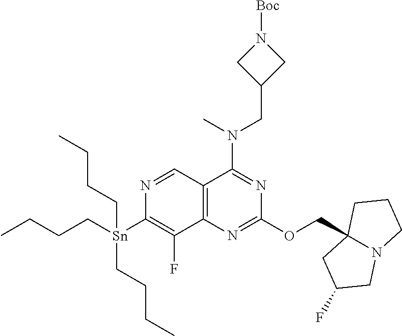 [SMILES: CCC[CH2][Sn]([CH2]CCC)([CH2]CCC)[c]1ncc2c(N(C)CC3CN(C(=O)OC(C)(C)C)C3)nc(OC[C@@]34CCCN3C[C@H](F)C4)nc2c1F]

### Step 1: tert-butyl 3-(((8-fluoro-2-(((2R,7aS)-2-fluorotetrahydro-1H-pyrrolizin-7a(5H)-yl)methoxy)-7-(tributylstannyl)pyrido[4,3-d]pyrimidin-4-yl)(methyl)amino)methyl)azetidine-1-carboxylate

The tin reagent was prepared in a similar fashion to Example #71, Step 5. The reaction mixture was concentrated in vacuo and the crude residue was purified by column chromatography (silica gel, 100-200 mesh, 0-100% EtOAc in petroleum ether) affording tert-butyl 3-(((8-fluoro-2-(((2R,7aS)-2-fluorotetrahydro-1H-pyrrolizin-7a(5H)-yl)methoxy)-7-(tributylstannyl)pyrido[4,3-d]pyrimidin-4-yl)(methyl)amino)methyl)azetidine-1-carboxylate (1.2 g, 81.50%) as a white solid. LCMS Rt=0.737 min, m/z=795.4 [M+H]⁺.

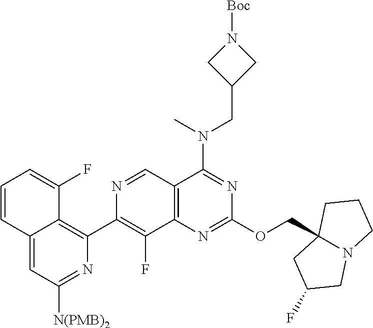

### Step 2: tert-butyl 3-(((7-(3-(bis(4-methoxybenzyl)amino)-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude product was purified by reverse phase HPLC (column: Phenomenex Luna C18 250*50 mm*10 um; mobile phase: [water(TFA)-ACN]; B %: 40%-70%, 10 min) affording tert-butyl 3-(((7-(3-(bis(4-methoxybenzyl)amino)-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate (200 mg, 35.08%, trifluoroacetic salt) as a yellow solid. LCMS Rt=2.135 min, m/z=905.4 [M+H]⁺.

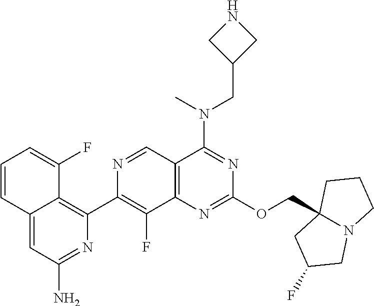 [SMILES: CN(CC1CNC1)c1nc(OC[C@@]23CCCN2C[C@H](F)C3)nc2c(F)c(-c3nc(N)cc4cccc(F)c34)ncc12]

### Step 3: 7-(3-amino-8-fluoroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The de-Boc and PMB protection was performed in a similar fashion to Example #71, Step 7. The crude product was purified by reverse phase HPLC (column: Phenomenex Luna 80*30 mm*3 um; mobile phase: [water(TFA)-ACN]; B %: 1%-30%, 8 min) affording 7-(3-amino-8-fluoroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (108 mg, 99.41%, trifluoroacetic salt) as a brown gum. LCMS Rt=0.527 min, m/z=565.3 [M+H]⁺.

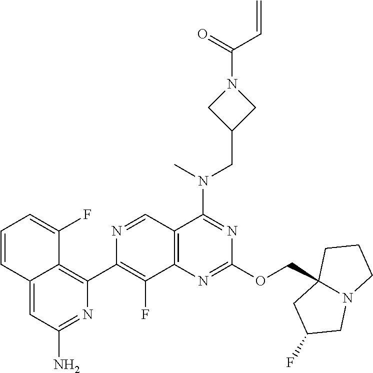 [SMILES: C=CC(=O)N1CC(CN(C)c2nc(OC[C@@]34CCCN3C[C@H](F)C4)nc3c(F)c(-c4nc(N)cc5cccc(F)c45)ncc23)C1]

### Step 4: 1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

The acylation reaction was performed in a similar fashion to Example #71, Step 11. The crude product was purified by reverse phase HPLC (column: Waters Xbridge BEH C18 100*30 mm*10 um; mobile phase: [water(NH₄HCO₃)-ACN]; B %: 25%-50%, 10 min) affording 1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one (4.72 mg, 13.31%) as a yellow oil: ¹H NMR (400 MHz, CD₃CN) δ 9.00-8.93 (m, 1H), 7.53-7.47 (m, 2H), 6.90 (d, J=2.4 Hz, 1H), 6.87-6.79 (m, 1H), 6.78-6.66 (m, 1H), 6.28-6.19 (m, 1H), 5.76-5.57 (m, 1H), 5.38-5.20 (m, 1H), 5.17 (s, 2H), 4.23-4.15 (m, 1H), 4.14-4.04 (m, 1H), 4.02-3.92 (m, 1H), 3.91-3.77 (m, 1H), 3.74-3.64 (m, 1H), 3.59-3.51 (m, 1H), 3.42-3.28 (m, 1H), 3.23-3.13 (m, 5H), 3.10-3.05 (m, 1H), 3.00 (s, 1H), 2.97-2.87 (m, 1H), 2.48-2.35 (m, 1H), 2.15-2.11 (m, 1H), 2.08 (s, 1H), 2.03 (s, 1H), 1.94-1.78 (m, 3H). LCMS Rt=2.480 min, m/z=619.3 [M+H]⁺.

LCMS (5 to 95% acetonitrile in water+0.03% ammonium bicarbonate over 6 mins) retention time 2.480 min, ESI+ found [M+H]⁺=619.3.

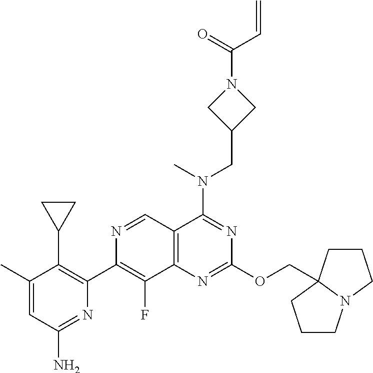 [SMILES: C=CC(=O)N1CC(CN(C)c2nc(OCC34CCCN3CCC4)nc3c(F)c(-c4nc(N)cc(C)c4C4CC4)ncc23)C1]

### Example 131: 1-(3-(((7-(6-amino-3-cyclopropyl-4-methylpyridin-2-yl)-8-fluoro-2-((tetrahydro-1H-pyrrolizin-7a(5H)-yl)methoxy)pyrido[4,3-d]pyrimidin-4-yl)(methyl)amino)methyl)azetidin-1-yl)prop-2-en-1-one

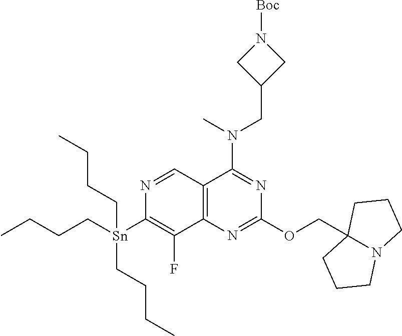 [SMILES: CCC[CH2][Sn]([CH2]CCC)([CH2]CCC)[c]1ncc2c(N(C)CC3CN(C(=O)OC(C)(C)C)C3)nc(OCC34CCCN3CCC4)nc2c1F]

### Step 1: tert-butyl 3-(((8-fluoro-2-((tetrahydro-1H-pyrrolizin-7a(5H)-yl)methoxy)-7-(tributylstannyl)pyrido[4,3-d]pyrimidin-4-yl)(methyl)amino)methyl)azetidine-1-carboxylate

The tin reagent was prepared in a similar fashion to Example #71, Step 5. The reaction mixture was concentrated in vacuo and the crude residue was purified by column chromatography (silica gel, 100-200 mesh, 0-100% EtOAc in petroleum ether) affording tert-butyl 3-(((8-fluoro-2-((tetrahydro-1H-pyrrolizin-7a(5H)-yl)methoxy)-7-(tributylstannyl)pyrido[4,3-d]pyrimidin-4-yl)(methyl)amino)methyl)azetidine-1-carboxylate (0.7 g, 94.05%) as a white solid. LCMS Rt=0.799 min, m/z=777.4 [M+H]⁺.

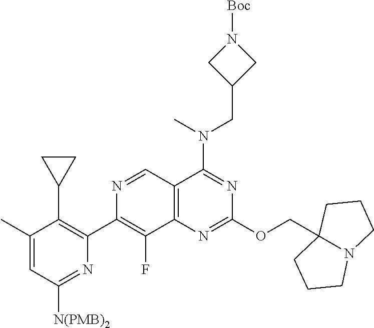

### Step 1: tert-butyl 3-(((7-(6-(bis(4-methoxybenzyl)amino)-3-cyclopropyl-4-methylpyridin-2-yl)-8-fluoro-2-((tetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude product was purified by reverse phase HPLC (column: Phenomenex Luna 80*30 mm*3 um; mobile phase: [water(TFA)-ACN]; B %: 35%-65%, 8 min) affording tert-butyl 3-(((7-(6-(bis(4-methoxybenzyl)amino)-3-cyclopropyl-4-methylpyridin-2-yl)-8-fluoro-2-((tetrahydro-1H-pyrrolizin-7a(5H)-yl)methoxy)pyrido[4,3-d]pyrimidin-4-yl)(methyl)amino)methyl)azetidine-1-carboxylate (70 mg, 12.44%, trifluoroacetic salt) as a white solid. LCMS Rt=2.439 min, m/z=873.5 [M+H]⁺.

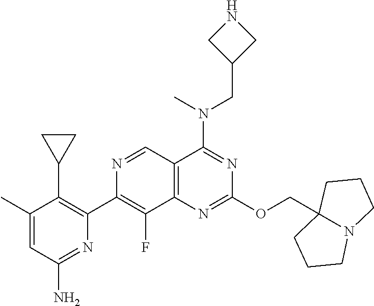 [SMILES: Cc1cc(N)nc(-c2ncc3c(N(C)CC4CNC4)nc(OCC45CCCN4CCC5)nc3c2F)c1C1CC1]

### Step 2: 7-(6-amino-3-cyclopropyl-4-methylpyridin-2-yl)-N-(azetidin-3-ylmethyl)-8-fluoro-N-methyl-2-((tetrahydro-1H-pyrrolizin-7a(5H)-yl)methoxy)pyrido[4,3-d]pyrimidin-4-amine

The Boc and PMB deprotection reaction was performed in a similar fashion to Example #71, Step #7. The crude product was purified by reverse phase HPLC (column: Phenomenex Luna 80*30 mm*3 um; mobile phase: [water(TFA)-ACN]; B %: 1%-30%, 8 min) affording 7-(6-amino-3-cyclopropyl-4-methylpyridin-2-yl)-N-(azetidin-3-ylmethyl)-8-fluoro-N-methyl-2-((tetrahydro-1H-pyrrolizin-7a(5H)-yl)methoxy)pyrido[4,3-d]pyrimidin-4-amine (20 mg, 45.00%, trifluoroacetic salt) as a white solid. LCMS Rt=1.166 min, m/z=533.3 [M+H]⁺

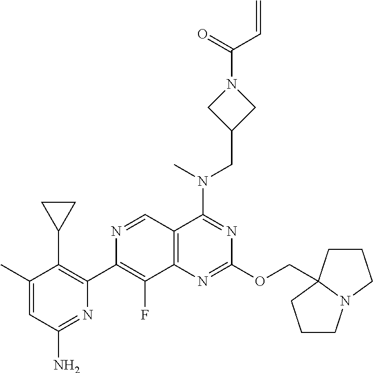 [SMILES: C=CC(=O)N1CC(CN(C)c2nc(OCC34CCCN3CCC4)nc3c(F)c(-c4nc(N)cc(C)c4C4CC4)ncc23)C1]

### Step 3: 1-(3-(((7-(6-amino-3-cyclopropyl-4-methylpyridin-2-yl)-8-fluoro-2-((tetrahydro-1H-pyrrolizin-7a(5H)-yl)methoxy)pyrido[4,3-d]pyrimidin-4-yl)(methyl)amino)methyl)azetidin-1-yl)prop-2-en-1-one

The acylation reaction was performed in a similar fashion to Example #71, Step 11. The crude product was purified by reverse phase HPLC (column: Phenomenex Luna C18 75*30 mm*3 um; mobile phase: [water(FA)-ACN]; B %: 1%-30%, 8 min) affording 1-(3-(((7-(6-amino-3-cyclopropyl-4-methylpyridin-2-yl)-8-fluoro-2-((tetrahydro-1H-pyrrolizin-7a(5H)-yl)methoxy)pyrido[4,3-d]pyrimidin-4-yl)(methyl)amino)methyl)azetidin-1-yl)prop-2-en-1-one (5 mg, 31.45%, formate salt) as a white solid: ¹H NMR (400 MHz, CD₃CN) δ 9.24 (s, 1H), 8.29-8.23 (m, 1H), 6.47 (s, 1H), 6.33-6.24 (m, 1H), 6.18-6.11 (m, 1H), 5.62 (dd, J=2.3, 10.3 Hz, 1H), 4.78 (s, 1H), 4.36 (s, 2H), 4.34-4.31 (m, 1H), 4.16 (d, J=7.5 Hz, 2H), 4.12-4.07 (m, 1H), 3.87 (dd, J=5.7, 10.1 Hz, 1H), 3.57 (s, 3H), 3.33-3.25 (m, 2H), 3.23-3.12 (m, 1H), 2.89-2.82 (m, 2H), 2.39 (s, 3H), 2.09 (td, J=2.6, 5.4 Hz, 2H), 2.01-1.96 (m, 3H), 1.87-1.80 (m, 3H), 1.76-1.66 (m, 2H), 0.56-0.49 (m, 2H), −0.01 (d, J=4.5 Hz, 2H). LCMS Rt=1.797 min, m/z=587.3 [M+H]⁺.

LCMS (5 to 95% acetonitrile in water+0.1% TFA over 6 mins) retention time 1.797 min, ESI+ found [M+H]⁺=587.3.

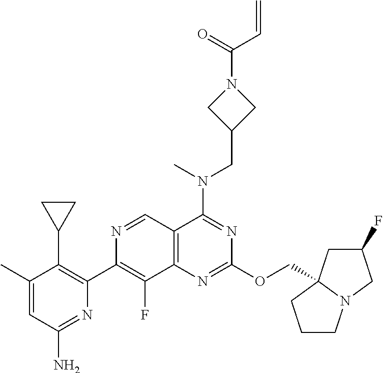 [SMILES: C=CC(=O)N1CC(CN(C)c2nc(OC[C@@]34CCCN3C[C@H](F)C4)nc3c(F)c(-c4nc(N)cc(C)c4C4CC4)ncc23)C1]

### Example 133: 1-(3-(((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

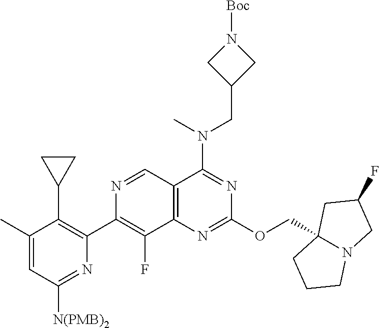

### Step 1: tert-butyl 3-(((7-(6-(bis(4-methoxybenzyl)amino)-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude product was purified by column chromatography (silica gel, 100-200 mesh, 0-20% MeOH in DCM) affording tert-butyl 3-(((7-(6-(bis(4-methoxybenzyl)amino)-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate (650 mg, crude) as a yellow gum. LCMS Rt=0.848 min, m/z=891.5 [M+H]⁺.

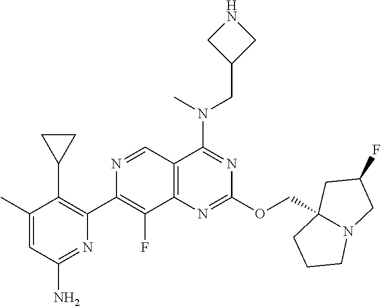 [SMILES: Cc1cc(N)nc(-c2ncc3c(N(C)CC4CNC4)nc(OC[C@@]45CCCN4C[C@H](F)C5)nc3c2F)c1C1CC1]

### Step 2: 7-(6-amino-3-cyclopropyl-4-methylpyridin-2-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The deprotection of Boc and PMB reaction was performed in a similar fashion to Example #71, Step 7. The crude product was purified by reverse phase HPLC (column: Phenomenex Luna 80*30 mm*3 um; mobile phase: [water(TFA)-ACN]; B %: 2%-32%, 8 min) affording 7-(6-amino-3-cyclopropyl-4-methylpyridin-2-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (30 mg, 13.41%, trifluoroacetic salt) as a yellow solid. LCMS Rt=0.403 min, m/z=551.3 [M+H]⁺.

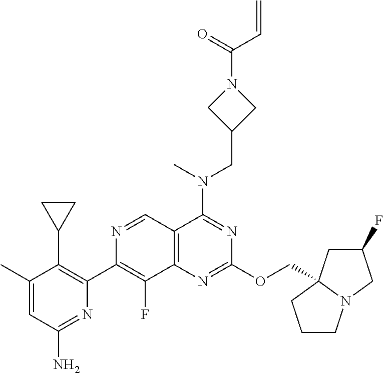 [SMILES: C=CC(=O)N1CC(CN(C)c2nc(OC[C@@]34CCCN3C[C@H](F)C4)nc3c(F)c(-c4nc(N)cc(C)c4C4CC4)ncc23)C1]

### Step 3: 1-(3-(((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

The acylation reaction was performed in a similar fashion to Example #71, Step 11. The crude product was purified by reverse phase HPLC (column: Waters Xbridge BEH C18 100*30 mm*10 um; mobile phase: [water(NH₄HCO₃)-ACN]; B %: 20%-45%, 8 min) affording 1-(3-(((7-(6-amino-3-cyclopropyl-4-methylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one (1.8 mg, 6.03%) as a yellow solid: ¹H NMR (400 MHz, CD₃CN) δ 9.21 (s, 1H), 6.46 (s, 1H), 6.34-6.22 (m, 1H), 6.18-6.11 (m, 1H), 5.62 (dd, J=2.2, 10.2 Hz, 1H), 5.19 (d, J=2.1 Hz, 1H), 4.75 (s, 1H), 4.37-4.27 (m, 1H), 4.20-4.06 (m, 6H), 3.84 (dd, J=5.6, 10.1 Hz, 1H), 3.54 (s, 3H), 3.22-3.05 (m, 4H), 2.89 (d, J=6.9 Hz, 1H), 2.41-2.36 (m, 3H), 2.10-2.02 (m, 2H), 1.91-1.63 (m, 6H), 0.58-0.49 (m, 2H), 0.00 (d, J=4.5 Hz, 2H). LCMS Rt=2.375 min, m/z=605.3 [M+H]⁺.

LCMS (5 to 95% acetonitrile in water+0.03% ammonium bicarbonate over 6 mins) retention time 2.375 min, ESI+ found [M+H]⁺=605.3.

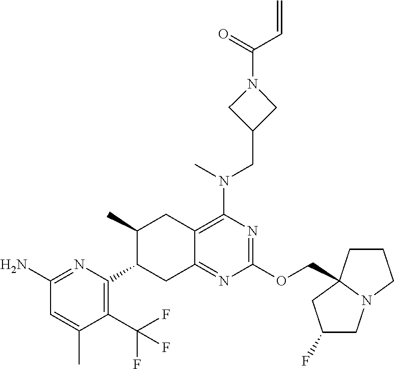 [SMILES: C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1]

### Example 137: 1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)prop-2-en-1-one

### Step 1: 1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)prop-2-en-1-one

The acylation reaction was performed in a similar fashion to Example #71, Step 11. The crude product was purified by reverse phase HPLC (neutral condition, column: Waters Xbridge Prep OBD C18 150*40 mm*10 um; mobile phase: [water(NH₄HCO₃)-ACN]; B %: 40%-70%, 8 min) affording 1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)prop-2-en-1-one (21.28 mg, 45.32%) as a yellow amorphous solid: ¹H NMR (400 MHz, CD₃CN) δ 6.35-6.22 (m, 2H), 6.20-6.10 (m, 1H), 5.67-5.58 (m, 1H), 5.36-5.16 (m, 3H), 4.28 (q, J=8.5 Hz, 1H), 4.10-3.97 (m, 3H), 3.95-3.85 (m, 2H), 3.80-3.57 (m, 1H), 3.52-3.36 (m, 1H), 3.22-3.09 (m, 3H), 3.07-2.94 (m, 6H), 2.93-2.78 (m, 2H), 2.70-2.61 (m, 1H), 2.53-2.43 (m, 1H), 2.38 (q, J=3.3 Hz, 3H), 2.15-2.06 (m, 3H), 2.04-1.99 (m, 1H), 1.93-1.77 (m, 3H), 0.76 (d, J=6.4 Hz, 3H). LCMS Rt=2.838 min, m/z=632.3 [M+H]⁺.

LCMS (5 to 95% acetonitrile in water+0.03% ammonium bicarbonate over 6 mins) retention time 2.838 min, ESI+ found [M+H]⁺=632.3

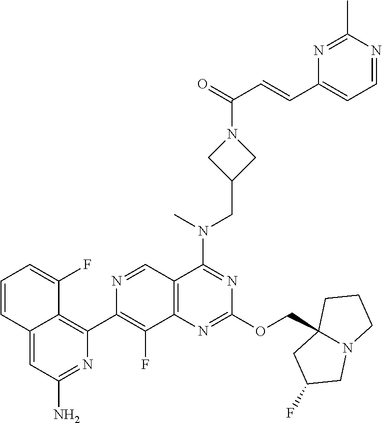 [SMILES: Cc1nccc(/C=C/C(=O)N2CC(CN(C)c3nc(OC[C@@]45CCCN4C[C@H](F)C5)nc4c(F)c(-c5nc(N)cc6cccc(F)c56)ncc34)C2)n1]

### Example 141: (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one

### Step 1: (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one

The amide coupling reaction was performed in a similar fashion to Example #71, Step 11. The crude product was purified by reverse phase HPLC (column: Phenomenex Luna C18 75*30 mm*3 um; mobile phase: [water(FA)-ACN]; B %: 5%-40%, 8 min) affording (E)-1-(3-(((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one (6 mg, 5.27%, formate salt) as a yellow solid: ¹H NMR (400 MHz, CD₃CN) δ 9.31-9.24 (m, 1H), 8.69 (d, J=5.1 Hz, 1H), 7.53-7.47 (m, 2H), 7.44-7.39 (m, 1H), 7.34 (d, J=5.1 Hz, 1H), 7.27-7.22 (m, 1H), 6.92 (d, J=2.5 Hz, 1H), 6.84 (ddd, J=2.3, 6.2, 12.6 Hz, 1H), 5.36-5.16 (m, 3H), 4.47 (t, J=8.4 Hz, 1H), 4.29-4.24 (m, 2H), 4.23-4.14 (m, 3H), 3.93 (dd, J=5.6, 10.2 Hz, 1H), 3.60 (s, 3H), 3.27-3.14 (m, 3H), 3.13-3.09 (m, 1H), 2.96-2.87 (m, 1H), 2.67 (s, 3H), 2.28-2.03 (m, 4H), 1.93-1.82 (m, 3H). LCMS Rt=2.358 min, m/z=711.3 [M+H]⁺.

LCMS (5 to 95% acetonitrile in water+0.03% ammonium bicarbonate over 6 mins) retention time 2.358 min, ESI+ found [M+H]⁺=710.3.

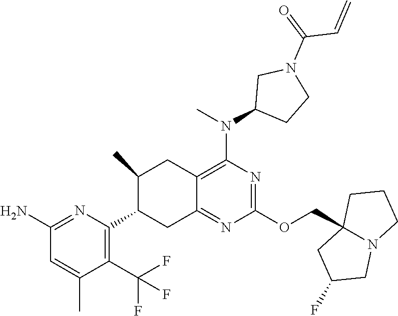 [SMILES: C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1]

### Example 145: 1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)prop-2-en-1-one

### Step 1: 1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)prop-2-en-1-one

The acylation reaction was performed in a similar fashion to Example #71, Step 11Example #71. The crude product was purified by reverse phase HPLC (neutral condition, column: Waters Xbridge BEH C18 100*30 mm*10 um; mobile phase: [water(NH₄HCO₃)-ACN]; B %: 35%-65%, 8 min) affording 1-((R)-3-(((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)prop-2-en-1-one (8.21 mg, 21.45%) as a yellow amorphous solid: ¹H NMR (400 MHz, CD₃CN) δ 6.60 (dd, J=10.4, 16.8 Hz, 1H), 6.31-6.18 (m, 2H), 5.72-5.61 (m, 1H), 5.35-5.14 (m, 3H), 4.76-4.55 (m, 1H), 4.07-3.89 (m, 3H), 3.85-3.71 (m, 1H), 3.62-3.50 (m, 1H), 3.44-3.31 (m, 1H), 3.22-3.11 (m, 3H), 3.07 (s, 1H), 3.03-2.97 (m, 1H), 2.95-2.80 (m, 5H), 2.68-2.60 (m, 1H), 2.55-2.45 (m, 1H), 2.38 (d, J=3.5 Hz, 3H), 2.28 (s, 1H), 2.15 (s, 1H), 2.08 (s, 2H), 2.04 (dd, J=2.8, 9.6 Hz, 2H), 1.93-1.77 (m, 3H), 0.77 (d, J=6.4 Hz, 3H). LCMS Rt=2.908 min, m/z=632.3 [M+H]⁺.

LCMS (5 to 95% acetonitrile in water+0.03% ammonium bicarbonate over 6 mins) retention time 2.908 min, ESI+ found [M+H]⁺=632.3.

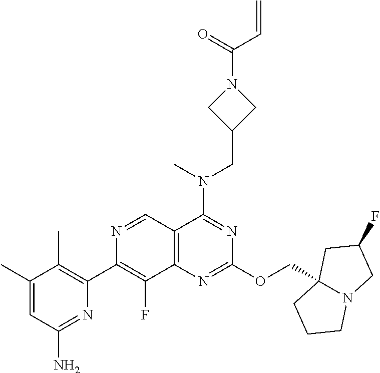 [SMILES: C=CC(=O)N1CC(CN(C)c2nc(OC[C@@]34CCCN3C[C@H](F)C4)nc3c(F)c(-c4nc(N)cc(C)c4C)ncc23)C1]

### Example 146: 1-(3-(((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

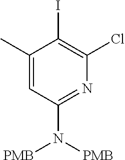

### Step 1: 6-chloro-5-iodo-N,N-bis(4-methoxybenzyl)-4-methylpyridin-2-amine

To a solution of 6-chloro-N,N-bis[(4-methoxyphenyl)methyl]-4-methyl-pyridin-2-amine (10 g, 26.12 mmol) in DMF (100 mL) was added N-Iodosuccinimide (5.88 g, 26.12 mmol) and the resulting mixture was stirred at 25° C. for 3 h. The crude residue was diluted with water (200 mL) and the resulting precipitate was collected by filtration to afford 6-chloro-5-iodo-N,N-bis(4-methoxybenzyl)-4-methylpyridin-2-amine (11 g, crude) as a yellow solid which was used in next step without further purification. LCMS Rt=1.048 min, m/z=509.0 [M+H]⁺

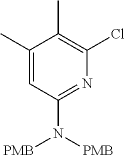

### Step 2: 6-chloro-N,N-bis(4-methoxybenzyl)-4,5-dimethylpyridin-2-amine

To a solution of 6-chloro-5-iodo-N,N-bis(4-methoxybenzyl)-4-methylpyridin-2-amine (3 g, 5.90 mmol) in 1,4-dioxane (30 mL) and water (3 mL) was added methylboronic acid (423.56 mg, 7.08 mmol), Cs₂CO₃ (5.76 g, 17.69 mmol) and Pd(dppf)Cl₂ DCM (431.45 mg, 589.65 umol). The resulting mixture was sparged under positive nitrogen pressure before stirring at 100° C. for 12 h.

After this time, the mixture was diluted with water (20 mL), extracted with EtOAc (3×50 mL), dried over sodium sulfate, concentrated in vacuo and purified by column chromatography (silica gel, 100-200 mesh, 0-20% EtOAc in petroleum ether) to afford 6-chloro-N,N-bis(4-methoxybenzyl)-4,5-dimethylpyridin-2-amine (1.8 g, 69.73%) as a white solid. LCMS Rt=1.048 min, m/z=397.2 [M+H]⁺

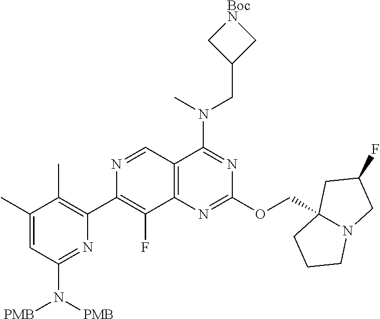

### Step 3: tert-butyl 3-(((7-(6-(bis(4-methoxybenzyl)amino)-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude residue was purified by column chromatography (silica gel, 100-200 mesh, 0-80% EtOAc in petroleum ether) affording tert-butyl 3-(((7-(6-(bis(4-methoxybenzyl)amino)-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate (0.68 g, 49.01%) as a brown oil. LCMS Rt=2.282 min, m/z=865.5 [M+H]⁺.

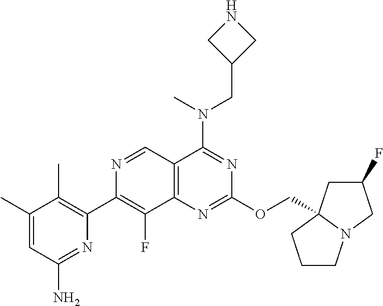 [SMILES: Cc1cc(N)nc(-c2ncc3c(N(C)CC4CNC4)nc(OC[C@@]45CCCN4C[C@H](F)C5)nc3c2F)c1C]

### Step 3: 7-(6-amino-3,4-dimethylpyridin-2-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The deprotection of Boc and PMB reaction was performed in a similar fashion to Example #71, Step 7. The reaction mixture was concentrated in vacuo affording 7-(6-amino-3,4-dimethylpyridin-2-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (147 mg, crude, trifluoroacetic salt) as a brown oil which was used in the next step without any further purification. LCMS Rt=0.385 min, m/z=525.3 [M+H]⁺.

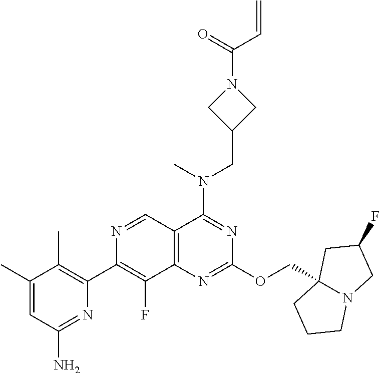 [SMILES: C=CC(=O)N1CC(CN(C)c2nc(OC[C@@]34CCCN3C[C@H](F)C4)nc3c(F)c(-c4nc(N)cc(C)c4C)ncc23)C1]

### Step 4: 1-(3-(((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one

The acylation reaction was performed in a similar fashion to Example #71, Step 11. The crude product was purified by reverse phase HPLC (column: Waters Xbridge Prep OBD C18 150*40 mm*10 um; mobile phase: [water(NH₄HCO₃)-ACN]; B %: 20%-50%, 8 min) affording 1-(3-(((7-(6-amino-3,4-dimethylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)prop-2-en-1-one (7.58 mg, 11.46%) as a pale yellow amorphous solid: ¹H NMR (400 MHz, CD₃CN) δ 9.20 (s, 1H), 6.50-6.43 (m, 1H), 6.31-6.11 (m, 2H), 5.66-5.57 (m, 1H), 5.36-5.15 (m, 1H), 4.72 (s, 2H), 4.37-4.27 (m, 1H), 4.23-4.02 (m, 6H), 3.88-3.78 (m, 1H), 3.58-3.50 (m, 3H), 3.24-3.10 (m, 3H), 3.09-3.03 (m, 1H), 2.94-2.84 (m, 1H), 2.27-2.13 (m, 6H), 2.12-1.99 (m, 3H), 1.91-1.78 (m, 3H). LCMS Rt=2.261 min, m/z=579.3 [M+H]⁺.

LCMS (5 to 95% acetonitrile in water+0.03% ammonium bicarbonate over 6 mins) retention time 2.261 min, ESI+ found [M+H]⁺=579.3.

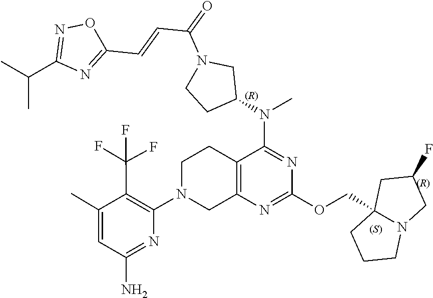

### Example 147: (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-isopropyl-1,2,4-oxadiazol-5-yl)prop-2-en-1-one

### Step 1: (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-isopropyl-1,2,4-oxadiazol-5-yl)prop-2-en-1-one

The amide coupling reaction was performed in a similar fashion to Example #71, Step 11. The crude product was purified by reverse phase HPLC (column: Waters Xbridge Prep OBD C¹⁸ 150*40 mm*10 um; mobile phase: [water(NH₄HCO₃)-ACN]; B %: 60%-90%, 8 min) affording (E)-1-((R)-3-((7-(6-amino-4-methyl-3-(trifluoromethyl)pyridin-2-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)pyrrolidin-1-yl)-3-(3-isopropyl-1,2,4-oxadiazol-5-yl)prop-2-en-1-one (18.82 mg, 21.67%) as a white solid: ¹H NMR (400 MHz, CD₃OD) δ 7.47-7.23 (m, 2H), 6.03 (s, 1H), 5.38-5.24 (m, 1H), 5.19-5.10 (m, 2H), 4.89-4.67 (m, 1H), 4.24-4.18 (m, 2H), 4.06 (d, J=8.0 Hz, 2H), 3.97-3.84 (m, 2H), 3.83-3.57 (m, 2H), 3.52-3.34 (m, 2H), 3.33-3.23 (m, 1H), 3.13-3.07 (m, 2H), 3.06-3.01 (m, 1H), 2.98-2.90 (m, 3H), 2.90-2.79 (m, 2H), 2.77-2.67 (m, 1H), 2.34-2.26 (m, 3H), 2.25-2.17 (m, 2H), 2.08-2.00 (m, 2H), 1.88-1.74 (m, 4H), 1.35-1.28 (m, 6H). LCMS Rt=2.296 min, m/z=729.4 [M+H]⁺.

LCMS (5 to 95% acetonitrile in water+0.03% ammonium bicarbonate over 6 mins) retention time 2.296 min, ESI+ found [M+H]⁺=729.4.

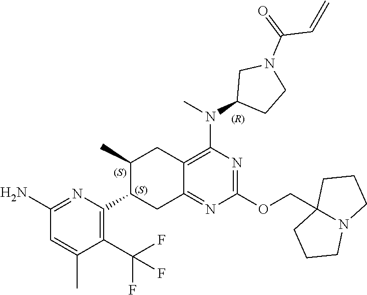

### Example 148: 1-((R)-3-(((6S,7S)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one

### Step 1: 1-((R)-3-(((6S,7S)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one

The acylation reaction was performed in a similar fashion to Example #71, Step 11Example #71. The crude product was purified by reverse phase prep-HPLC (column: Waters Xbridge BEH C¹⁸ 100*30 mm*10 um; mobile phase: [water(NH₄HCO₃)-ACN]; B %: 35%-70%, 8 min) affording 1-((R)-3-(((6S,7S)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one (13.43 mg, 28.73%) as a yellow oil: ¹H NMR (400 MHz, CD₃CN) δ 6.59 (dd, J=10.3, 16.8 Hz, 1H), 6.34-6.16 (m, 2H), 5.67 (ddd, J=2.4, 5.0, 10.4 Hz, 1H), 5.20 (s, 2H), 4.79-4.49 (m, 1H), 4.10-3.89 (m, 3H), 3.84-3.70 (m, 1H), 3.63-3.50 (m, 1H), 3.44-3.30 (m, 1H), 3.17 (td, J=5.2, 10.6 Hz, 1H), 2.96 (dd, J=4.9, 9.7 Hz, 3H), 2.92 (d, J=4.6 Hz, 3H), 2.88-2.80 (m, 1H), 2.69-2.56 (m, 3H), 2.54-2.45 (m, 1H), 2.38 (q, J=3.5 Hz, 3H), 2.15-2.02 (m, 3H), 1.94-1.87 (m, 2H), 1.86-1.70 (m, 4H), 1.65-1.54 (m, 2H), 0.77 (d, J=6.4 Hz, 3H). LCMS Rt=2.818 min, m/z=614.3 [M+H]⁺.

LCMS (5 to 95% acetonitrile in water+0.1% ammonium bicarbonate over 6 mins) retention time 2.818 min, ESI+ found [M+H]⁺=614.3.

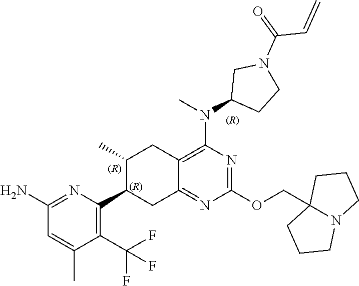

### Example 149: 1-((R)-3-(((6R,7R)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one

### Step 1: 1-((R)-3-(((6R,7R)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one

The acylation reaction was performed in a similar fashion to Example #71, Step 11. The crude product was purified by reverse phase prep-HPLC (column: Waters Xbridge BEH C¹⁸ 100*30 mm*10 um; mobile phase: [water(NH₄HCO₃)-ACN]; B %: 25%-70%, 8 min) affording 1-((R)-3-(((6R,7R)-7-(6-amino-4-methyl-3-(trifluoromethyl)pyridin-2-yl)-6-methyl-2-((tetrahydro-1H-pyrrolizin-7a(5H)-yl)methoxy)-5,6,7,8-tetrahydroquinazolin-4-yl)(methyl)amino)pyrrolidin-1-yl)prop-2-en-1-one (16.48 mg, 45.23%) as a yellow solid: ¹H NMR (400 MHz, CD₃CN) δ 6.68-6.45 (m, 1H), 6.35-6.13 (m, 2H), 5.72-5.57 (m, 1H), 5.20 (s, 2H), 4.62 (td, J=8.5, 17.2 Hz, 1H), 4.01 (s, 2H), 3.93-3.70 (m, 2H), 3.65-3.45 (m, 1H), 3.24-3.13 (m, 1H), 3.07-2.97 (m, 3H), 2.91 (s, 3H), 2.89-2.81 (m, 1H), 2.73-2.60 (m, 3H), 2.52-2.41 (m, 2H), 2.38 (q, J=3.3 Hz, 3H), 2.16 (s, 3H), 1.95-1.91 (m, 2H), 1.89-1.74 (m, 4H), 1.64 (dd, J=6.6, 12.6 Hz, 2H), 0.78 (d, J=5.9 Hz, 3H). LCMS Rt=2.869 min, m/z=614.3 [M+H]⁺.

LCMS (5 to 95% acetonitrile in water+0.1% ammonium bicarbonate over 6 mins) retention time 2.869 min, ESI+ found [M+H]⁺=614.3.

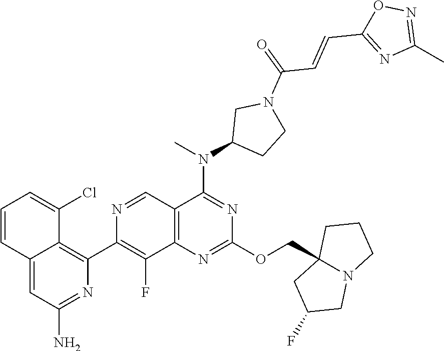 [SMILES: Cc1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1]

### Example 150: (E)-1-((R)-3-((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

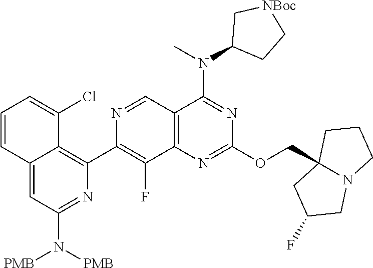

### Step 1: tert-butyl (R)-3-((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The crude product was purified by reverse phase HPLC (column: Phenomenex Luna 80*30 mm*3 um; mobile phase: [water(TFA)-ACN]; B %: 35%-75%, 8 min) affording tert-butyl (R)-3-((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (60 mg, 22.48%) as a yellow solid. LCMS Rt=0.904 min, m/z=921.4 [M+H]⁺.

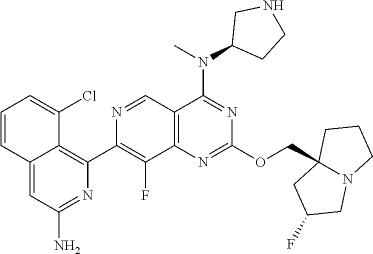 [SMILES: CN(c1nc(OC[C@@]23CCCN2C[C@H](F)C3)nc2c(F)c(-c3nc(N)cc4cccc(Cl)c34)ncc12)[C@@H]1CCNC1]

### Step 2: 7-(3-amino-8-chloroisoquinolin-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB reaction was performed in a similar fashion to Example #71, Step 7. The reaction mixture was concentrated in vacuo affording 7-(3-amino-8-chloroisoquinolin-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (30 mg, crude, trifluoroacetic salt) as a yellow oil which was used in the next step without any further purification. LCMS Rt=0.514 min, m/z=581.2 [M+H]⁺.

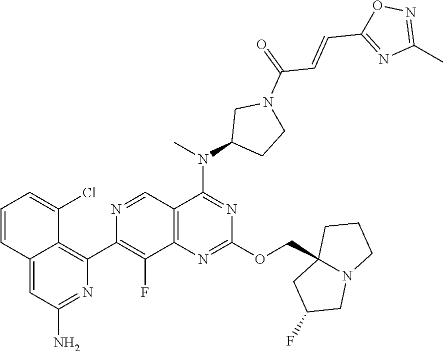 [SMILES: Cc1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1]

### Step 3: (E)-1-((R)-3-((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one

The amide coupling reaction was performed in a similar fashion to Example #71, Step 11. The crude product was purified by reverse phase HPLC (column: Waters Xbridge Prep OBD C18 150*40 mm*10 um; mobile phase: [water(NH₄HCO₃)-ACN]; B %: 25%-55%, 8 min) affording (E)-1-((R)-3-((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-methyl-1,2,4-oxadiazol-5-yl)prop-2-en-1-one (8.57 mg, 27.10%) as a pale yellow amorphous solid: ¹H NMR (400 MHz, CD₃CN) δ 9.17 (s, 1H), 7.63 (d, J=8.4 Hz, 1H), 7.50-7.33 (m, 3H), 7.23 (d, J=7.3 Hz, 1H), 6.92 (s, 1H), 5.44-5.29 (m, 2H), 5.21-5.13 (m, 2H), 4.23-4.13 (m, 2H), 4.04-3.96 (m, 1H), 3.90-3.72 (m, 2H), 3.66-3.44 (m, 2H), 3.42 (s, 3H), 3.13-3.05 (m, 2H), 2.93-2.82 (m, 1H), 2.38 (d, J=9.1 Hz, 3H), 2.13-2.01 (m, 5H), 1.89-1.80 (m, 3H). LCMS Rt=2.014 min, m/z=717.3 [M+H]⁺.

LCMS (5 to 95% acetonitrile in water+0.03% ammonium bicarbonate over 6 mins) retention time 2.014 min, ESI+ found [M+H]⁺=717.3.

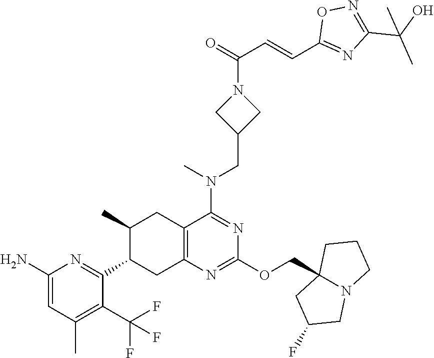 [SMILES: Cc1cc(N)nc([C@H]2Cc3nc(OC[C@@]45CCCN4C[C@H](F)C5)nc(N(C)CC4CN(C(=O)/C=C/c5nc(C(C)(C)O)no5)C4)c3C[C@@H]2C)c1C(F)(F)F]

### Example 155: (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one

### Step 1: (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one

The amide coupling reaction was performed in a similar fashion to Example #71, Step 11. The crude product was purified by reverse phase prep-HPLC (column: Waters Xbridge Prep OBD C18 150*40 mm*10 um; mobile phase: [water(NH₄HCO₃)-ACN]; B %: 30%-65%, 8 min) affording (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one (20.54 mg, 30.18%) as a yellow solid. ¹H NMR (400 MHz, CD₃CN) δ 7.36-7.26 (m, 1H), 7.21-7.10 (m, 1H), 6.27 (s, 1H), 5.36-5.15 (m, 3H), 4.40 (q, J=8.3 Hz, 1H), 4.21-4.06 (m, 2H), 4.06-3.98 (m, 2H), 3.94-3.79 (m, 2H), 3.71 (dd, J=5.8, 10.4 Hz, 1H), 3.52-3.41 (m, 1H), 3.21-3.15 (m, 1H), 3.14-3.07 (m, 3H), 3.04 (m, 4H), 3.01-2.94 (m, 1H), 2.92-2.78 (m, 2H), 2.69-2.62 (m, 1H), 2.49 (dd, J=11.4, 15.6 Hz, 1H), 2.37 (q, J=3.4 Hz, 3H), 2.16-2.11 (m, 1H), 2.10-2.04 (m, 2H), 2.03-1.99 (m, 1H), 1.91-1.84 (m, 1H), 1.83-1.74 (m, 2H), 1.59 (d, J=2.3 Hz, 6H), 0.76 (d, J=6.4 Hz, 3H). LCMS Rt=3.016 min, m/z=758.4 [M+H]⁺.

LCMS (5 to 95% acetonitrile in water+0.03% ammonium bicarbonate over 6 mins) retention time 3.016 min, ESI+ found [M+H]⁺=758.4.

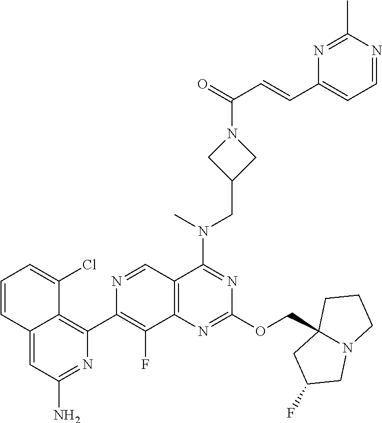 [SMILES: Cc1nccc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1]

### Example 156: (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one

### Step 1 (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one

The HWE reaction was performed in a similar fashion to Example #2, Step 6. The crude product was purified by reverse phase HPLC (column: Waters Xbridge Prep OBD C18 150*40 mm*10 um; mobile phase: [water(NH₄HCO₃)-ACN]; B %: 20%-50%, 8 min) affording (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(2-methylpyrimidin-4-yl)prop-2-en-1-one (8.73 mg, 30.38%) as a yellow amorphous solid: ¹H NMR (400 MHz, CD₃CN) δ 9.27-9.21 (m, 1H), 8.67 (d, J=5.1 Hz, 1H), 7.64 (d, J=8.3 Hz, 1H), 7.45-7.37 (m, 2H), 7.32 (d, J=5.1 Hz, 1H), 7.26-7.20 (m, 2H), 6.92 (s, 1H), 5.15 (s, 3H), 4.46 (s, 1H), 4.26 (s, 2H), 4.20-4.09 (m, 4H), 3.91 (dd, J=5.6, 9.9 Hz, 1H), 3.58 (s, 3H), 3.28-3.18 (m, 1H), 3.14-3.03 (m, 3H), 2.86 (s, 1H), 2.65 (s, 3H), 2.07 (s, 3H), 1.87-1.77 (m, 3H). LCMS Rt=2.435 min, m/z=727.3 [M+H]⁺.

LCMS (5 to 95% acetonitrile in water+0.03% ammonium bicarbonate over 6 mins) retention time 2.435 min, ESI+ found [M+H]=727.3.

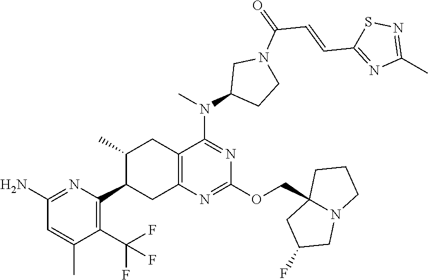 [SMILES: Cc1nsc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1]

### Example 158: (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one

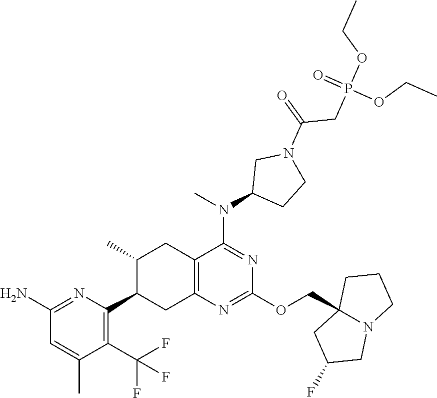 [SMILES: CCOP(=O)(CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1)OCC]

### Step 1: diethyl (2-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-2-oxoethyl)phosphonate

The amide coupling reaction was performed in a similar fashion to Example #2, Step 5. The crude product was purified by reverse phase prep-HPLC (column: Waters Xbridge BEH C18 100*30 mm*10 um; mobile phase: [water(NH₄HCO₃)-ACN]; B %: 20%-60%, 8 min) affording diethyl (2-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-2-oxoethyl)phosphonate (50 mg, 45.76%) as a white solid. LCMS Rt=2.023 min, m/z=756.4 [M+H]⁺.

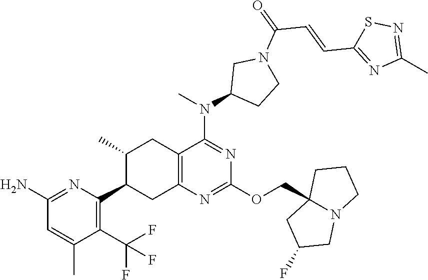 [SMILES: Cc1nsc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1]

### Step 2: (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one

The HWE reaction was performed in a similar fashion to Example #2, Step 6. The reaction mixture was purified by reverse phase HPLC (column: Waters Xbridge BEH C18 100*30 mm*10 um; mobile phase: [water(NH₄HCO₃)-ACN]; B %: 35%-73%, 8 min) affording (E)-1-((R)-3-(((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)pyrrolidin-1-yl)-3-(3-methyl-1,2,4-thiadiazol-5-yl)prop-2-en-1-one (3.32 mg, 8.60%) as a yellow amorphous solid: ¹H NMR (400 MHz, CD₃CN) δ 7.74 (dd, J=5.5, 15.4 Hz, 1H), 7.38-7.25 (m, 1H), 6.28 (s, 1H), 5.34-5.13 (m, 3H), 4.79-4.59 (m, 1H), 4.25-4.12 (m, 1H), 4.10-3.90 (m, 3H), 3.88-3.54 (m, 2H), 3.50-3.39 (m, 1H), 3.22-3.09 (m, 3H), 3.06 (d, J=7.9 Hz, 1H), 2.93 (d, J=5.0 Hz, 3H), 2.90-2.80 (m, 2H), 2.69-2.59 (m, 4H), 2.55-2.45 (m, 1H), 2.38 (q, J=3.3 Hz, 3H), 2.29-2.25 (m, 1H), 2.16-2.06 (m, 5H), 1.92-1.75 (m, 3H), 0.77 (d, J=6.3 Hz, 3H). LCMS Rt=3.012 min, m/z=730.3 [M+H]⁺.

LCMS (5 to 95% acetonitrile in water+0.03% ammonium bicarbonate over 6 mins) retention time 3.012 min, ESI+ found [M+H]⁺=730.3.

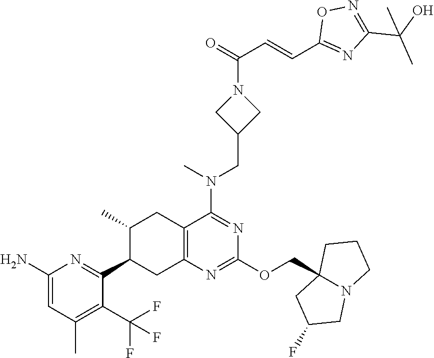 [SMILES: Cc1cc(N)nc([C@@H]2Cc3nc(OC[C@@]45CCCN4C[C@H](F)C5)nc(N(C)CC4CN(C(=O)/C=C/c5nc(C(C)(C)O)no5)C4)c3C[C@H]2C)c1C(F)(F)F]

### Example 162: (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one

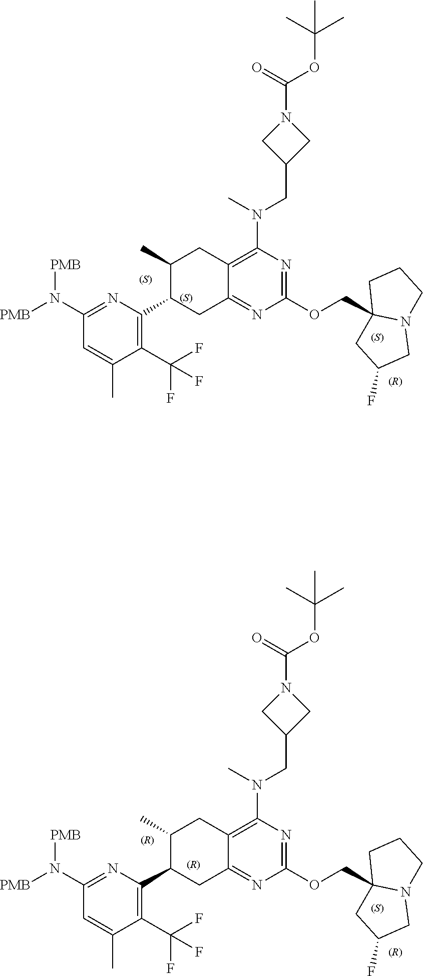

### Step 1: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate and tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate

The mixture of diastereomers was prepared in the same manner as Example #65 and was further purified by SFC to give arbitrarily assigned: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 1, retention time=1.687 min) (120 mg, 25.50%) as a white solid. LCMS Rt=0.670 min, m/z=918.5 [M+H]⁺. tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 2, retention time=2.228 min) (100 mg, 21.25%) as a white solid. LCMS Rt=0.670 min, m/z=918.5 [M+H]⁺. SFC (column: DAICEL CHIRALPAK IE(250 mm*30 mm,10 um); mobile phase: [ACN/EtOH(0.1% NH₃H₂O)]; B %: 62%-62%, 35 min).

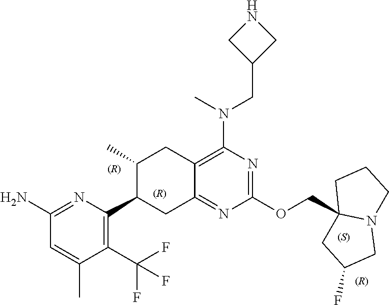

### Step 2: (6R,7R)-7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine

The Boc and PMB deprotections were performed in a similar fashion to Example #71, Step 7. The reaction mixture was concentrated in vacuo and purified by reverse phase HPLC (column: Phenomenex Luna 80*30 mm*3 um; mobile phase: [water(TFA)-ACN]; B %: 1%-30%, 8 min) affording (6R,7R)-7-(6-amino-4-methyl-3-(trifluoromethyl)pyridin-2-yl)-N-(azetidin-3-ylmethyl)-2-(((2R,7aS)-2-fluorohexahydro-1H-pyrrolizin-7a-yl)methoxy)-N,6-dimethyl-5,6,7,8-tetrahydroquinazolin-4-amine (100 mg, 92.91%, trifluoroacetate salt) as a white solid. LCMS Rt=0.434 min, m/z=578.3 [M+H]⁺.

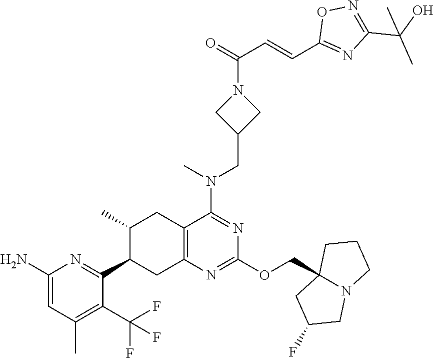 [SMILES: Cc1cc(N)nc([C@@H]2Cc3nc(OC[C@@]45CCCN4C[C@H](F)C5)nc(N(C)CC4CN(C(=O)/C=C/c5nc(C(C)(C)O)no5)C4)c3C[C@H]2C)c1C(F)(F)F]

### Step 3: (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one

The amide coupling reaction was performed in a similar fashion to Example #71, Step 11. The reaction mixture was concentrated in vacuo and purified by reverse phase HPLC (column: Waters Xbridge BEH C18 100*30 mm*10 um;mobile phase: [water(NH₄HCO₃)-ACN]; B %: 30%-60%, 8 min) affording (E)-1-(3-((((6R,7R)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-(2-hydroxypropan-2-yl)-1,2,4-oxadiazol-5-yl)prop-2-en-1-one (14.42 mg, 12.84%) as a yellow amorphous solid: ¹H NMR (400 MHz, CD₃CN) δ 7.35-7.28 (m, 1H), 7.20-7.12 (m, 1H), 6.27 (s, 1H), 5.33-5.14 (m, 3H), 4.40 (q, J=8.3 Hz, 1H), 4.21-4.06 (m, 2H), 4.06-3.97 (m, 2H), 3.96-3.81 (m, 2H), 3.77-3.62 (m, 1H), 3.47 (ddd, J=6.9, 14.0, 15.7 Hz, 1H), 3.21-3.15 (m, 1H), 3.12 (d, J=2.8 Hz, 3H), 3.02-2.95 (m, 1H), 2.92-2.78 (m, 2H), 2.67 (dd, J=4.4, 15.8 Hz, 1H), 2.49 (dd, J=11.4, 15.6 Hz, 2H), 2.37 (q, J=3.5 Hz, 3H), 2.33-2.17 (m, 3H), 2.15-2.12 (m, 1H), 2.10-2.04 (m, 2H), 2.01 (d, J=10.0 Hz, 1H), 1.92-1.85 (m, 1H), 1.84-1.74 (m, 2H), 1.59 (d, J=2.0 Hz, 6H), 0.76 (d, J=6.4 Hz, 3H). LCMS Rt=3.022 min, m/z=758.4 [M+H]⁺.

LCMS (5 to 95% acetonitrile in water+0.03% ammonium bicarbonate over 6 mins) retention time 3.022 min, ESI+ found [M+H]=758.4.

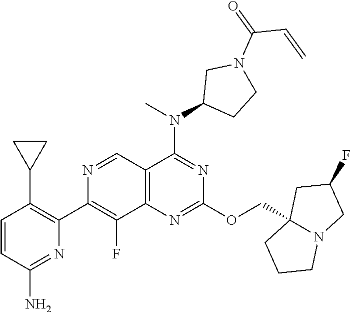 [SMILES: C=CC(=O)N1CC[C@@H](N(C)c2nc(OC[C@@]34CCCN3C[C@H](F)C4)nc3c(F)c(-c4nc(N)ccc4C4CC4)ncc23)C1]

### Example 166: 1-((R)-3-((7-(6-amino-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

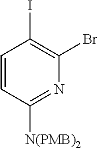

### Step 1: 6-bromo-5-iodo-N,N-bis(4-methoxybenzyl)pyridin-2-amine

To a solution of 6-bromo-5-iodo-pyridin-2-amine (1.65 g, 5.52 mmol) in DMF (10 mL) was added NaH (441.57 mg, 11.04 mmol, 60% dispersion in mineral oil) at 0° C. The resulting mixture was allowed to warm to 25° C. and stirring was continued for 1 h. After this time, 1-(chloromethyl)-4-methoxybenzene (1.56 g, 9.94 mmol) was added and the mixture was stirred at 25° C. for 1 h. The reaction mixture was then quenched with water (30 mL) at 0° C., the layers were separated and the aqueous phase was extracted with EtOAc (3×50 mL). The combined organic layers were dried over sodium sulfate and concentrated in vacuo. The crude residue was purified by column chromatography (silica gel, 100-200 mesh, 0-10% EtOAc in petroleum ether) affording 6-bromo-5-iodo-N,N-bis(4-methoxybenzyl)pyridin-2-amine (2.5 g, 78.45%) as a yellow oil: ¹H NMR (400 MHz, Chloroform-d) δ 7.58 (d, J=8.6 Hz, 1H), 7.18-7.11 (m, 4H), 6.85 (br d, J=8.5 Hz, 4H), 6.15 (d, J=8.6 Hz, 1H), 4.63 (s, 4H), 3.80 (s, 6H). LCMS Rt=1.037 min, m/z=539.0 [M+H]⁺.

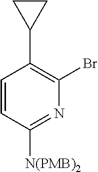

### Step 2: 6-bromo-5-cyclopropyl-N,N-bis(4-methoxybenzyl)pyridin-2-amine

To a solution of 6-bromo-5-iodo-N,N-bis[(4-methoxyphenyl)methyl]pyridin-2-amine (2.5 g, 4.64 mmol), cyclopropylboronic acid (406.23 mg, 4.73 mmol) and Cs₂CO₃ (4.53 g, 13.91 mmol) in 1,4-dioxane (10 mL) and water (1 mL) was added Pd(dppf)Cl₂ (678.51 mg, 927.29 umol). The resulting mixture was stirred at 100° C. for 3 h under nitrogen atmosphere. After this time, the mixture was cooled to room temperature, diluted with water (20 mL) and the aqueous phase was extracted with EtOAc (3×50 mL). The combined organic layers were dried over sodium sulfate and concentrated in vacuo. The residue was purified by reverse phase HPLC (column: Welch Xtimate C18 250*70 mm*10 um; mobile phase: [water(NH₄HCO₃)-ACN]; B %: 65%-95%, 17 min) affording 6-bromo-5-cyclopropyl-N,N-bis(4-methoxybenzyl)pyridin-2-amine (800 mg, 38.06%) as yellow oil: ¹H NMR (400 MHz, Chloroform-d) δ 7.17 (d, J=8.5 Hz, 4H), 6.94 (d, J=8.5 Hz, 1H), 6.87-6.81 (m, 4H), 6.27 (d, J=8.5 Hz, 1H), 4.64 (s, 4H), 3.80 (s, 6H), 1.95 (s, 1H), 0.95-0.89 (m, 2H), 0.54 (d, J=5.5 Hz, 2H). LCMS Rt=3.036 min, m/z=453.1 [M+H]⁺.

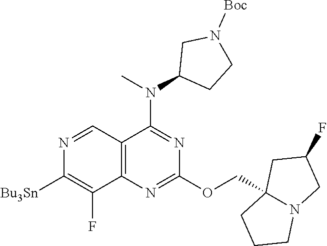

### Step 3: (R)-tert-butyl 3-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)pyrrolidine-1-carboxylate

The tin reagent was prepared in a similar fashion to Example #71, Step 5. The crude residue was purified by column chromatography (silica gel, 100-200 mesh, 0-50% tetrahydrofuran in petroleum ether) affording (R)-tert-butyl 3-((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)pyrrolidine-1-carboxylate (2.3 g, 70.30%) as an orange oil. LCMS Rt=0.801 min, m/z=795.4 [M+H]⁺.

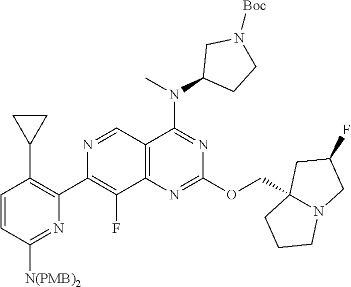

### Step 4: tert-butyl (R)-3-((7-(6-(bis(4-methoxybenzyl)amino)-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The crude residue was purified by column chromatography (silica gel, 100-200 mesh, 95-100% EtOAc in petroleum ether) affording tert-butyl (R)-3-((7-(6-(bis(4-methoxybenzyl)amino)-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (250 mg, 24.07%) as an orange oil, which was used in the next step without any further purification. LCMS Rt=0.863 min, m/z=877.5 [M+H]⁺

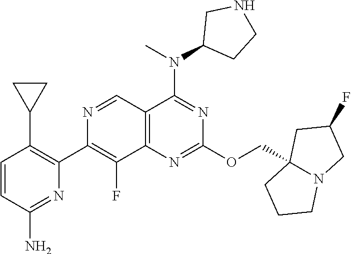 [SMILES: CN(c1nc(OC[C@@]23CCCN2C[C@H](F)C3)nc2c(F)c(-c3nc(N)ccc3C3CC3)ncc12)[C@@H]1CCNC1]

### Step 5: 7-(6-amino-3-cyclopropylpyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB was performed in a similar fashion to Example #71, Step 7. The crude product was purified by reverse phase HPLC (column: Phenomenex Luna 80*30 mm*3 um;mobile phase: [water(TFA)-ACN];B %: 1%-35%,8 min) affording 7-(6-amino-3-cyclopropylpyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (180 mg, 98.06%) as a yellow oil. LCMS Rt=1.074 min, m/z=537.3 [M+H]⁺

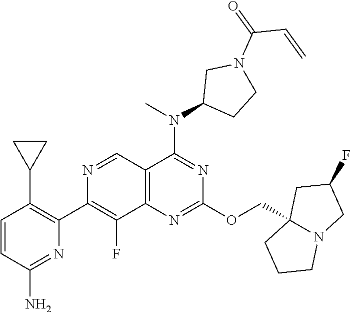 [SMILES: C=CC(=O)N1CC[C@@H](N(C)c2nc(OC[C@@]34CCCN3C[C@H](F)C4)nc3c(F)c(-c4nc(N)ccc4C4CC4)ncc23)C1]

### Step 6: 1-((R)-3-((7-(6-amino-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one

The amide coupling reaction was performed in a similar fashion to Example #71, Step 11. The crude product was purified by reverse phase HPLC (column: Waters Xbridge Prep OBD C18 150*40 mm*10 um;mobile phase:[water(NH₄HCO₃)-ACN];B %: 15%-55%,8 min) affording 1-((R)-3-((7-(6-amino-3-cyclopropylpyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)prop-2-en-1-one (23.5 mg, 21.35%) as a white amorphous solid: ¹H NMR (400 MHz, CD₃CN) δ 9.18 (d, J=2.0 Hz, 1H), 7.17 (d, J=8.6 Hz, 1H), 6.64-6.49 (m, 2H), 6.27-6.17 (m, 1H), 5.66 (ddd, J=2.3, 7.3, 10.0 Hz, 1H), 5.42-5.15 (m, 2H), 4.83 (s, 2H), 4.21-4.01 (m, 2H), 3.95-3.81 (m, 1H), 3.68-3.60 (m, 1H), 3.57-3.41 (m, 1H), 3.39 (s, 3H), 3.18-3.08 (m, 2H), 3.06 (s, 1H), 2.93-2.84 (m, 1H), 2.40-2.22 (m, 2H), 2.15-2.01 (m, 3H), 1.92-1.77 (m, 3H), 1.70-1.61 (m, 1H), 1.33 (d, J=12.1 Hz, 1H), 0.68-0.61 (m, 2H), 0.49-0.43 (m, 2H). LCMS Rt=2.528 min, m/z=591.3 [M+H]⁺.

LCMS (5 to 95% acetonitrile in water+0.03% ammonium bicarbonate over 6 mins) retention time 2.528 min, ESI+ found [M+H]=591.3.

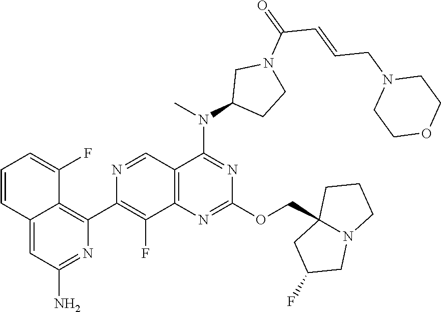 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1]

### Example 167: (E)-1-((R)-3-((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one

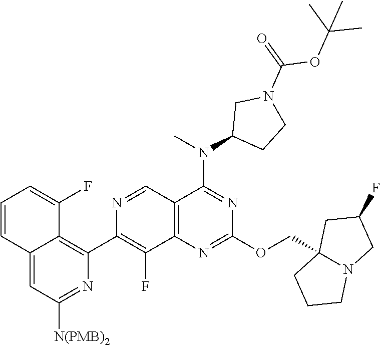

### Step 1: tert-butyl (R)-3-((7-(3-(bis(4-methoxybenzyl)amino)-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The resulting crude residue was purified by column chromatography (silica gel, 100-200 mesh, 0-100% EtOAc in petroleum ether) affording tert-butyl (R)-3-((7-(3-(bis(4-methoxybenzyl)amino)-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (1.5 g, 73.08%) as a brown oil. LCMS Rt=2.675 min, m/z=905.4 [M+H]⁺.

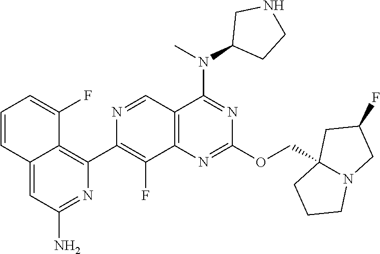 [SMILES: CN(c1nc(OC[C@@]23CCCN2C[C@H](F)C3)nc2c(F)c(-c3nc(N)cc4cccc(F)c34)ncc12)[C@@H]1CCNC1]

### Step 2: 7-(3-amino-8-fluoroisoquinolin-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB was performed in a similar fashion to Example #71, Step 7. The crude product was purified by reverse phase HPLC (column: Phenomenex Luna 80*30 mm*3 um;mobile phase: [water(TFA)-ACN]; B %: 1%-40%, 8 min) affording 7-(3-amino-8-fluoroisoquinolin-1-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (50 mg, 66.68%, trifluoroacetate salt) as a yellow solid. LCMS Rt=1.019 min, m/z=565.3 [M+H]⁺.

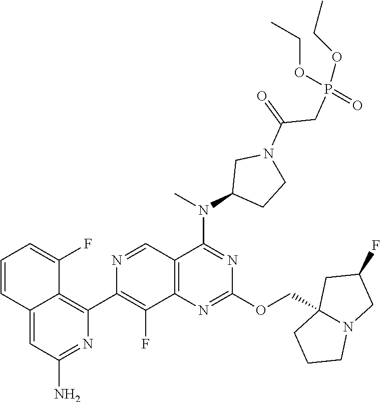 [SMILES: CCOP(=O)(CC(=O)N1CC[C@@H](N(C)c2nc(OC[C@@]34CCCN3C[C@H](F)C4)nc3c(F)c(-c4nc(N)cc5cccc(F)c45)ncc23)C1)OCC]

### Step 3: diethyl (2-((R)-3-((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-oxoethyl)phosphonate

The coupling reaction was performed in a similar fashion to Example #2, Step 5. The crude product was purified by reverse phase HPLC (column: Phenomenex Luna 80*30 mm*3 um;mobile phase: [water(TFA)-ACN]; B %: 5%-35%, 8 min) affording diethyl (2-((R)-3-((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-oxoethyl)phosphonate (20 mg, 13.18%, trifluoroacetate salt) as a red solid. LCMS Rt=1.171 min, m/z=743.3 [M+H]⁺.

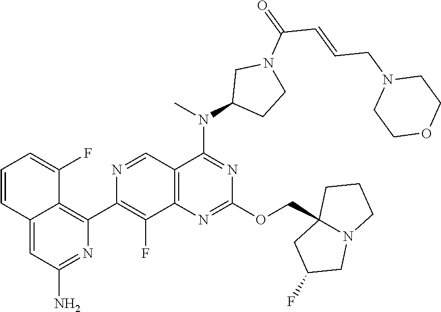 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1]

### Step 4: (E)-1-((R)-3-((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one

The HWE reaction was performed in a similar fashion to Example #2, Step 6. The crude product was purified by reverse phase HPLC (column: Phenomenex Luna C18 75*30 mm*3 um; mobile phase: [water(FA)-ACN]; B %: 5%-35%, 8 min) affording (E)-1-((R)-3-((7-(3-amino-8-fluoroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-4-morpholinobut-2-en-1-one (1.1 mg, 10.70%, formate salt) as a yellow oil: ¹H NMR (400 MHz, DMSO-d6) δ 9.32-9.19 (m, 1H), 8.79 (d, J=2.1 Hz, 1H), 8.43 (s, 1H), 7.98-7.72 (m, 2H), 7.28 (dd, J=7.6, 12.3 Hz, 1H), 6.85 (td, J=5.8, 15.5 Hz, 1H), 6.52 (d, J=15.4 Hz, 1H), 5.40-5.20 (m, 2H), 4.23-4.01 (m, 3H), 3.64-3.58 (m, 6H), 3.16-3.07 (m, 6H), 3.02 (s, 2H), 2.89-2.78 (m, 2H), 2.70-2.64 (m, 1H), 2.34 (dd, J=1.8, 3.7 Hz, 4H), 2.13 (s, 1H), 2.08-1.98 (m, 3H), 1.88-1.75 (m, 4H). LCMS Rt=1.657 min, m/z=718.3 [M+H]⁺.

LCMS (5% to 35% acetonitrile in water+0.03% formic acid over 8 mins) retention time 1.657 min, ESI+ found [M+H]=718.3.

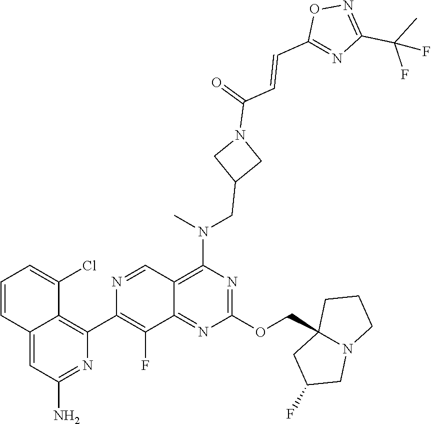 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12]

### Example 169: (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

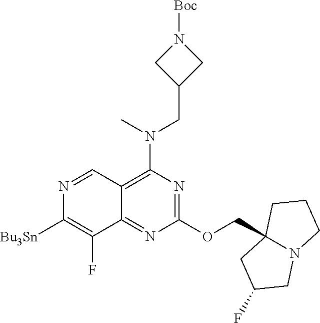

### Step 1: tert-butyl 3-(((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)methyl)azetidine-1-carboxylate

The tin reagent was prepared in a similar fashion to Example #71, Step 5. The crude residue was purified by column chromatography (silica gel, 100-200 mesh, 80-100% tetrahydrofuran in petroleum ether) affording tert-butyl 3-(((8-fluoro-2-(((2R,7aS)-2-fluorohexahydro-1H-pyrrolizin-7a-yl)methoxy)-7-(tributylstannyl)pyrido[4,3-d]pyrimidin-4-yl)(methyl)amino)methyl)azetidine-1-carboxylate (4 g, 62.10%) as a yellow oil. LCMS Rt=1.126 min, m/z=795.4 [M+H]⁺.

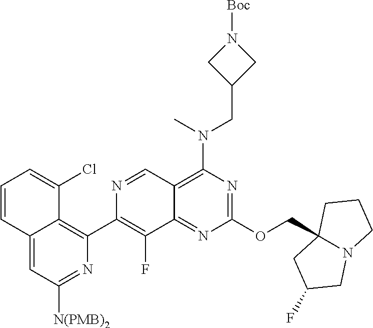

### Step 2: tert-butyl 3-(((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate

The Stille reaction was performed in a similar fashion to Example #71, Step 6. The crude product was purified by reverse phase HPLC (column: Phenomenex Luna C18 250*50 mm*10 um; mobile phase: [water(TFA)-ACN]; B %: 45%-75%, 10 min) affording tert-butyl 3-(((7-(3-(bis(4-methoxybenzyl)amino)-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidine-1-carboxylate (400 mg, 27.85%) as a yellow solid. LCMS Rt=0.887 min, m/z=921.4 [M+H]⁺.

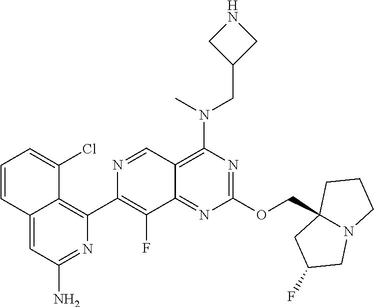 [SMILES: CN(CC1CNC1)c1nc(OC[C@@]23CCCN2C[C@H](F)C3)nc2c(F)c(-c3nc(N)cc4cccc(Cl)c34)ncc12]

### Step 3: 7-(3-amino-8-chloroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine

The deprotection of Boc and PMB was performed in a similar fashion to Example #71, Step 7. The mixture was concentrated in vacuo affording 7-(3-amino-8-chloroisoquinolin-1-yl)-N-(azetidin-3-ylmethyl)-8-fluoro-2-(((2R,7aS)-2-fluorotetrahydro-1H-pyrrolizin-7a(5H)-yl)methoxy)-N-methylpyrido[4,3-d]pyrimidin-4-amine (100 mg, crude) as a yellow oil, used in next step without further purification. LCMS Rt=0.511 min, m/z=581.2 [M+H]⁺.

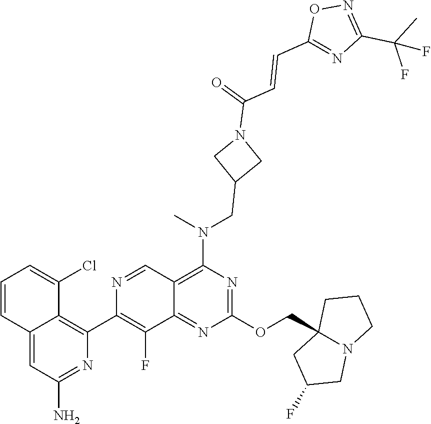 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12]

### Step 4: (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one

The amide coupling reaction was performed in a similar fashion to Example #71, Step 11. The crude product was purified by reverse phase HPLC (column: Waters Xbridge Prep OBD C18 150*40 mm*10 um; mobile phase: [water(NH₄HCO₃)-ACN]; B %: 30%-60%, 8 min) affording (E)-1-(3-(((7-(3-amino-8-chloroisoquinolin-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)methyl)azetidin-1-yl)-3-(3-(1,1-difluoroethyl)-1,2,4-oxadiazol-5-yl)prop-2-en-1-one (11.06 mg, 12.37%) as a yellow amorphous solid: ¹H NMR (400 MHz, CD₃CN) δ 9.27-9.20 (m, 1H), 7.63 (d, J=8.4 Hz, 1H), 7.45-7.35 (m, 2H), 7.26 (d, J=16.0 Hz, 2H), 6.92 (s, 1H), 5.33-5.12 (m, 3H), 4.48 (t, J=8.8 Hz, 1H), 4.28-4.09 (m, 6H), 3.97-3.91 (m, 1H), 3.57 (s, 3H), 3.22 (d, J=6.9 Hz, 1H), 3.16-3.10 (m, 2H), 3.06 (s, 1H), 2.91-2.86 (m, 1H), 2.08 (s, 3H), 2.03 (s, 3H), 1.89-1.74 (m, 3H). LCMS Rt=2.701 min, m/z=767.3 [M+H]⁺.

LCMS (5 to 95% acetonitrile in water+0.03% ammonium bicarbonate over 6 mins) retention time 2.701 min, ESI+ found [M+H]=767.3.

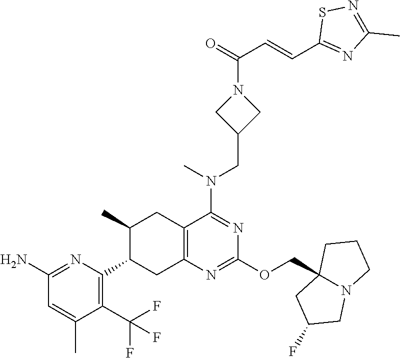 [SMILES: Cc1nsc(/C=C/C(=O)N2CC(CN(C)c3nc(OC[C@@]45CCCN4C[C@H](F)C5)nc4c3C[C@H](C)[C@@H](c3nc(N)cc(C)c3C(F)(F)F)C4)C2)n1]

### Example 174: (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one

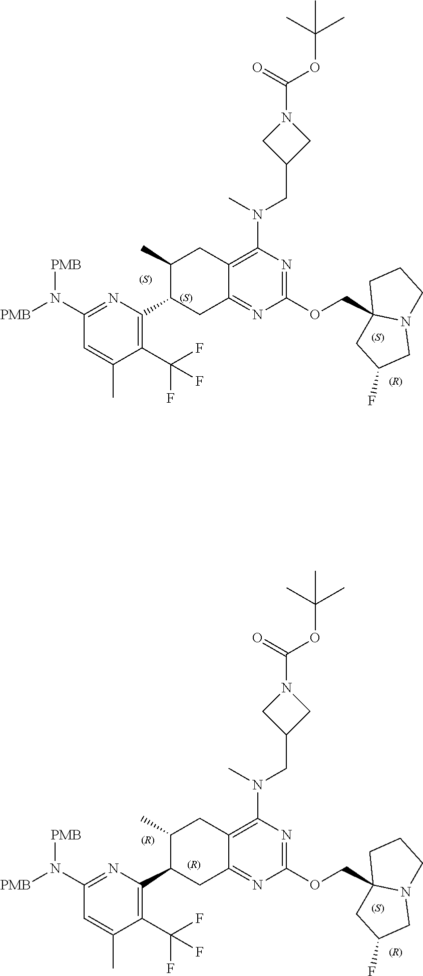

### Step 1: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate and tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate

The mixture of diastereomers was further purified by SFC to give arbitrarily assigned: tert-butyl 3-((((6S,7S)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 1, retention time=1.687 min) (120 mg, 25.50%) as a white solid. LCMS Rt=0.670 min, m/z=918.5 [M+H]⁺. tert-butyl 3-((((6R,7R)-7-(6-(bis(4-methoxybenzyl)amino)-4-methyl-3-(trifluoromethyl)pyridin-2-yl)-2-(((2R,7aS)-2-fluorohexahydro-1H-pyrrolizin-7a-yl)methoxy)-6-methyl-5,6,7,8-tetrahydroquinazolin-4-yl)(methyl)amino)methyl)azetidine-1-carboxylate (Peak 2, retention time=2.228 min) (100 mg, 21.25%) as a white solid. LCMS Rt=0.670 min, m/z=918.5 [M+H]⁺. SFC (column: DAICEL CHIRALPAK IE(250 mm*30 mm,10 um);mobile phase: [ACN/EtOH(0.1% NH₃H₂O)]; B %: 62%-62%, 35 min).

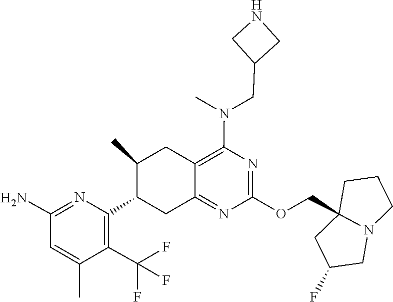 [SMILES: Cc1cc(N)nc([C@H]2Cc3nc(OC[C@@]45CCCN4C[C@H](F)C5)nc(N(C)CC4CNC4)c3C[C@@H]2C)c1C(F)(F)F]

### Step 2: (6S,7S)-7-(6-amino-4-methyl-3-(trifluoromethyl)pyridin-2-yl)-N-(azetidin-3-ylmethyl)-2-(((2R,7aS)-2-fluorotetrahydro-1H-pyrrolizin-7a(5H)-yl)methoxy)-N,6-dimethyl-5,6,7,8-tetrahydroquinazolin-4-amine

The Boc and PMB deprotections were performed in a similar fashion to Example #71, Step 7. The reaction mixture was concentrated in vacuo and purified by reverse phase HPLC (column: Phenomenex Luna 80*30 mm*3 um;mobile phase: [water(TFA)-ACN];B %: 5%-35%,8 min) affording (6S,7S)-7-(6-amino-4-methyl-3-(trifluoromethyl)pyridin-2-yl)-N-(azetidin-3-ylmethyl)-2-(((2R,7aS)-2-fluorotetrahydro-1H-pyrrolizin-7a(5H)-yl)methoxy)-N,6-dimethyl-5,6,7,8-tetrahydroquinazolin-4-amine (260 mg, 86.27%, trifluoroacetate salt) as a white solid. LCMS Rt=0.820 min, m/z=578.3 [M+H]⁺.

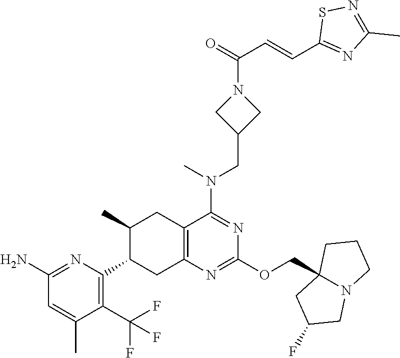 [SMILES: Cc1nsc(/C=C/C(=O)N2CC(CN(C)c3nc(OC[C@@]45CCCN4C[C@H](F)C5)nc4c3C[C@H](C)[C@@H](c3nc(N)cc(C)c3C(F)(F)F)C4)C2)n1]

### Step 3: (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one

The amide coupling reaction was performed in a similar fashion to Example #71, Step 11. The reaction mixture was concentrated in vacuo and purified by reverse phase HPLC (column: Waters Xbridge Prep OBD C18 150*40 mm*10 um; mobile phase: [water(NH₄HCO₃)-ACN]; B %: 30%-70%, 8 min) affording (E)-1-(3-((((6S,7S)-7-(6-amino-4-methyl-3-(trifluoromethyl)pyridin-2-yl)-2-(((2R,7aS)-2-fluorotetrahydro-1H-pyrrolizin-7a(5H)-yl)methoxy)-6-methyl-5,6,7,8-tetrahydroquinazolin-4-yl)(methyl)amino)methyl)azetidin-1-yl)-3-(3-methyl-1,2,4-thiadiazol-5-yl)prop-2-en-1-one (20.88 mg, 23.61%) as a white solid: ¹H NMR (400 MHz, CD₃CN) δ 6.81-6.62 (m, 1H), 6.16-5.99 (m, 1H), 5.33 (s, 1H), 4.44-4.16 (m, 3H), 3.45 (d, J=7.7 Hz, 1H), 3.30-3.02 (m, 4H), 3.00-2.71 (m, 2H), 2.64-2.44 (m, 1H), 2.18 (s, 3H), 2.14-2.01 (m, 5H), 2.00-1.82 (m, 2H), 1.79-1.66 (m, 4H), 1.63-1.50 (m, 1H), 1.44 (s, 3H), 1.31-1.28 (m, 1H), 1.23-1.16 (m, 2H), 1.12 (s, 2H), 0.98-0.80 (m, 3H), −0.17 (s, 3H). LCMS Rt=2.974 min, m/z=730.3 [M+H]⁺.

LCMS (5 to 95% acetonitrile in water+0.03% ammonium bicarbonate over 6 mins) retention time 2.974 min, ESI+ found [M+H]=730.3.

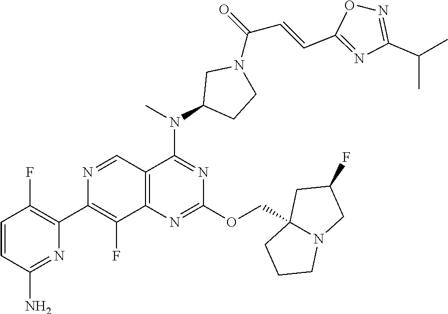 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5F)ncc34)C2)n1]

### Example 178: (E)-1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

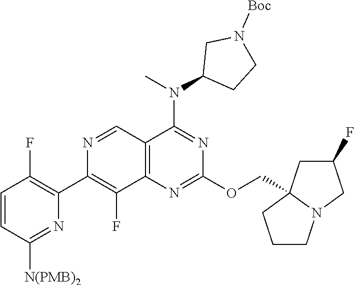

### Step 1: tert-butyl (R)-3-((7-(6-(bis(4-methoxybenzyl)amino)-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

The Stille reaction was performed in a similar fashion to Example #71, Step 6. The resulting crude residue was purified by column chromatography (silica gel, 100-200 mesh, 60-100% EtOAc in petroleum ether) affording tert-butyl (R)-3-((7-(6-(bis(4-methoxybenzyl)amino)-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (440 mg, 40.84%) as a brown oil.

LCMS Rt=2.557 min, m/z=855.4 [M+H]⁺.

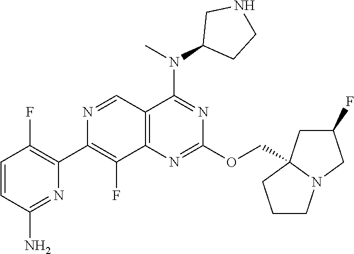 [SMILES: CN(c1nc(OC[C@@]23CCCN2C[C@H](F)C3)nc2c(F)c(-c3nc(N)ccc3F)ncc12)[C@@H]1CCNC1]

### Step 2: 7-(6-amino-3-fluoropyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

The deprotection of Boc and PMB was performed in a similar fashion to Example #71, Step 7. The crude product was purified by reverse phase HPLC (column: Waters Xbridge Prep OBD C18 150*40 mm*10 um;mobile phase:[water(NH₄HCO₃)-ACN];B %: 5%-40%,8 min) affording 7-(6-amino-3-fluoropyridin-2-yl)-8-fluoro-2-(((2R,7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (80 mg, 33.23%) as a brown oil.

LCMS Rt=0.558 min, m/z=515.2 [M+H]⁺

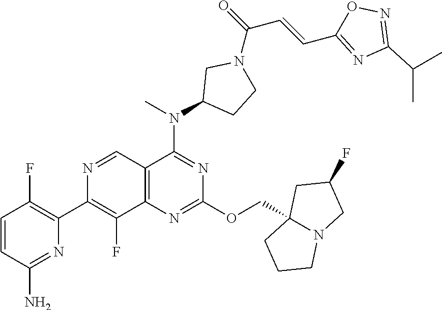 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5F)ncc34)C2)n1]

### Step 3: (E)-1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one

The amide coupling reaction was performed in a similar fashion to Example #71, Step 11. The crude product was purified by reverse phase HPLC (column: Waters Xbridge Prep OBD C18 150*40 mm*10 um;mobile phase: [water(NH₄HCO₃)-ACN]; B %: 30%-60%, 8 min) affording (E)-1-((R)-3-((7-(6-amino-3-fluoropyridin-2-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)pyrrolidin-1-yl)-3-(3-isopropyl-1,2,4-oxadiazol-5-yl)prop-2-en-1-one (2.8 mg, 6.07%) as a yellow oil: ¹H NMR (400 Hz, Acetonitrile-d3) δ 9.28-9.14 (m, 1H), 7.57-7.25 (m, 3H), 6.76-6.59 (m, 1H), 5.49-5.16 (m, 2H), 5.03 (br s, 1H), 4.29-4.16 (m, 2H), 4.01 (br s, 1H), 3.81-3.73 (m, 1H), 3.65-3.49 (m, 1H), 3.48-3.35 (m, 3H), 3.23-3.08 (m, 4H), 2.98-2.89 (m, 1H), 2.49-2.31 (m, 3H), 2.12-2.05 (m, 2H), 1.90 (br d, J=5.3 Hz, 3H), 1.34 (dd, J=7.0, 8.5 Hz, 6H). LCMS Rt=2.757 min, m/z=679.3 [M+H]⁺.

LCMS (5 to 95% acetonitrile in water+0.03% ammonium bicarbonate over 6 mins) retention time 2.757 min, ESI+ found [M+H]⁺=679.3.

TABLE A
Physicochemical characterization data for additional compounds synthesized by
variations of the methods described herein

| | | | LC/MSS |
|---|---|---|---|
| NR | Structure | NMR | m/z Rt |
| 77 | 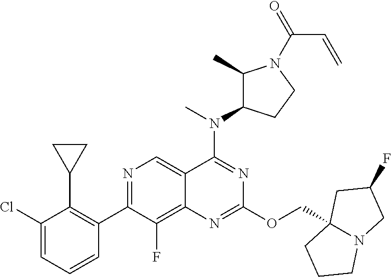 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C] | ¹H NMR (400 MHz, CDCl₃) δ 9.22 (s, 1H), 7.51-7.47 (m, 1H), 7.40-7.37 (m, 1H), 7.33- 7.30 (m, 1H), 6.51-6.42 (m, 2H), 5.74- 5.71 (m, 1H), 5.40-5.22 (m, 1H), 5.04-4.91 (m, 2H), 4.37-4.18 (m, 2H), 3.93-3.78 (m, 2H), 3.62 (s, 3H), 3.38-3.15 (m, 3H), 3.08- 2.94 (m, 1H), 2.42-2.33 (m, 4H), 2.10-2.02 (m, 3H), 1.97 (s, 1H), 1.23-1.08 (m, 4H), 0.81-0.66 (m, 2H), 0.26-0.11 (m, 2H). | 623.3 3.151 min |
| 78 | 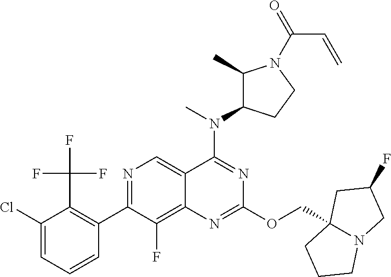 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C] | ¹H NMR (400 MHz, CDCl₃) δ 9.23-9.09 (m, 1H), 7.70-7.63 (m, 1H), 7.61-7.52 (m, 1H), 7.39-7.31 (m, 1H), 6.54-6.35 (m, 2H), 5.78- 5.67 (m, 1H), 5.39-5.18 (m, 1H), 5.07- 4.87 (m, 2H), 4.37-4.09 (m, 2H), 3.94-3.77 (m, 1H), 3.72-3.54 (m, 4H), 3.33-3.09 (m, 3H), 3.05-2.91 (m, 1H), 2.62-2.32 (m, 2H), 2.31-2.03 (m, 3H), 2.01-1.80 (m, 3H), 1.22- 1.06 (m, 3H). | 651.2 3.098 min |
| 82 | 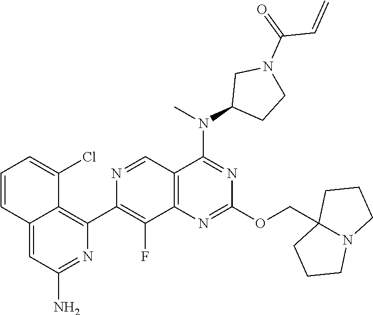 [SMILES: C=CC(=O)N1CC[C@@H](N(C)c2nc(OCC34CCCN3CCC4)nc3c(F)c(-c4nc(N)cc5cccc(Cl)c45)ncc23)C1] | ¹H NMR (400 MHz, CD₃CN) δ 9.19 (s, 1H), 7.66 (d, J = 7.9 Hz, 1H), 7.44 (dd, J = 7.4, 8.4 Hz, 1H), 7.26 (dd, J = 1.0, 7.4 Hz, 1H), 6.97- 6.91 (m, 1H), 6.66-6.54 (m, 1H), 6.30-6.22 (m, 1H), 5.72-5.66 (m, 1H), 5.45-5.32 (m, 1H), 5.19 (s, 2H), 4.25-4.16 (m, 2H), 4.13- 3.94 (m, 1H), 3.93-3.79 (m, 1H), 3.72-3.64 (m, 1H), 3.60-3.47 (m, 1H), 3.43 (s, 3H), 3.07-2.99 (m, 2H), 2.66 (td, J = 6.8, 10.1 Hz, 2H), 2.47-2.26 (m, 4H), 1.86 (qd, J = 6.4, 16.0 Hz, 4H), 1.71-1.63 (m, 2H). | 617.3/618.3 2.267 min |
| 83 | 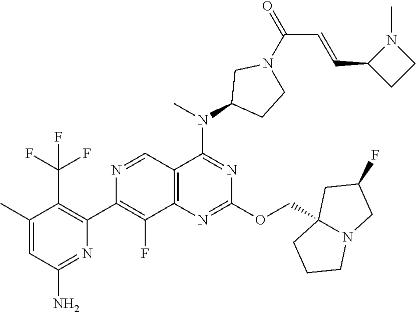 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F] | ¹H NMR (400 MHz, CD₃CN) δ 9.05-9.00 (m, 1H), 6.67 (td, J = 5.2, 15.1 Hz, 1H), 6.58- 6.45 (m, 1H), 6.33-6.19 (m, 1H), 5.44 (d, J = 6.6 Hz, 2H), 5.28-5.06 (m, 2H), 4.15- 4.07 (m, 1H), 4.06-4.01 (m, 1H), 3.98- 3.79 (m, 1H), 3.78-3.65 (m, 1H), 3.55-3.42 (m, 2H), 3.28 (d, J = 4.6 Hz, 3H), 3.24-3.16 (m, 1H), 3.05 (d, J = 5.8 Hz, 2H), 2.98 (s, 1H), 2.83-2.77 (m, 1H), 2.76-2.67 (m, 1H), 2.34 (s, 3H), 2.28-2.24 (m, 1H), 2.23-2.15 (m, 3H), 2.15-2.12 (m, 2H), 2.11-2.07 (m, 1H), 2.06-2.00 (m, 2H), 1.99-1.94 (m, 1H), 1.79 (dd, J = 10.3, 19.0 Hz, 4H) | 702.2 1.802 min |
| 85 | 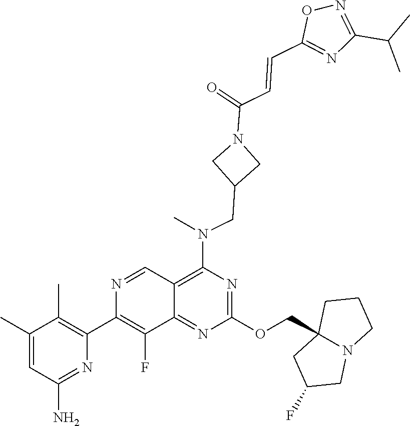 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C] | ¹H NMR (400 MHz, CD₃CN) δ 9.27-9.19 (m, 1H), 7.34-7.26 (m, 1H), 7.17-7.08 (m, 1H), 6.52-6.45 (m, 1H), 5.37-5.17 (m, 1H), 4.82-4.59 (m, 2H), 4.46 (t, J = 8.7 Hz, 1H), 4.29-4.21 (m, 2H), 4.21-4.10 (m, 4H), 3.95- 3.88 (m, 1H), 3.56 (s, 3H), 3.28-3.17 (m, 3H), 3.15-3.07 (m, 2H), 2.97 (s, 1H), 2.25 (s, 6H), 1.91-1.73 (m, 4H), 1.62-1.44 (m, 2H), 1.31 (d, J = 7.0 Hz, 6H) | 689.2 1.901 min |
| 86 | 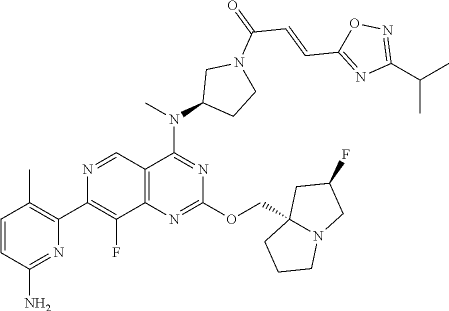 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F] | ¹H NMR (400 MHz, CD₃CN) δ 9.22 (s, 1H), 7.57-7.32 (m, 3H), 6.60 (d, J = 8.4 Hz, 1H), 5.48-5.21 (m, 2H), 4.90-4.79 (m, 2H), 4.33- 4.20 (m, 2H), 4.03 (dd, J = 8.4, 13.1 Hz, 1H), 3.95-3.84 (m, 1H), 3.82-3.74 (m, 1H), 3.66-3.51 (m, 1H), 3.44 (s, 3H), 3.32-3.08 (m, 4H), 3.03-2.92 (m, 1H), 2.48-2.30 (m, 4H), 2.24 (d, J = 2.8 Hz, 4H), 2.08 (s, 3H), 1.35 (dd, J = 7.1, 7.8 Hz, 6H) | 675.4 2.766 min |
| 87 | 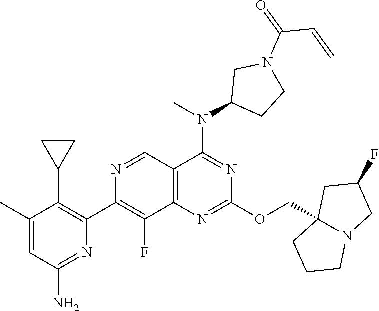 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1] | ¹H NMR (400 MHz, CD₃CN) δ 9.19-9.14 (m, 1H), 6.64-6.52 (m, 1H), 6.48-6.44 (m, 1H), 6.27-6.19 (m, 1H), 5.71-5.62 (m, 1H), 5.19 (s, 2H), 4.77-4.74 (m, 1H), 4.24-4.16 (m, 1H), 4.14-4.09 (m, 1H), 3.94 (dd, J = 8.0, 12.9 Hz, 1H), 3.90-3.76 (m, 1H), 3.69- 3.60 (m, 1H), 3.58-3.41 (m, 1H), 3.41-3.35 (m, 3H), 3.18-3.08 (m, 2H), 3.06 (s, 1H), 2.93-2.84 (m, 1H), 2.39 (s, 3H), 2.36 (d, J = 8.8 Hz, 1H), 2.31-2.25 (m, 1H), 2.19-2.16 (m, 1H), 2.11-1.99 (m, 3H), 1.91-1.82 (m, 3H), 1.74-1.65 (m, 1H), 0.56-0.47 (m, 2H), 0.03-−0.06 (m, 2H) | 605.4 2.615 min |
| 90 | 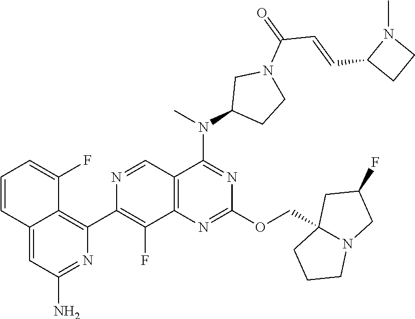 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ¹H NMR (400 MHz, CD₃CN) 9.22 (d, J = 2.6 Hz, 1H), 7.55-7.44 (m, 2H), 6.93 (d, J = 2.0 Hz, 1H), 6.89-6.74 (m, 2H), 6.45-6.35 (m, 1H), 5.40-5.32 (m, 1H), 5.24-5.15 (m, 2H), 4.29-4.21 (m, 1H), 4.17 (d, J = 2.3 Hz, 1H), 4.15-4.04 (m, 1H), 3.99-3.89 (m, 1H), 3.88- 3.76 (m, 1H), 3.71-3.47 (m, 3H), 3.44 (d, J = 1.9 Hz, 3H), 3.35-3.27 (m, 1H), 3.22- 3.14 (m, 2H), 3.13-3.07 (m, 1H), 2.96-2.78 (m, 2H), 2.44-2.29 (m, 3H), 2.26 (d, J = 7.9 Hz, 3H), 2.14 (s, 2H), 2.11-2.06 (m, 2H), 1.93-1.78 (m, 3H) | 688.4 1.768 min |
| 91 | 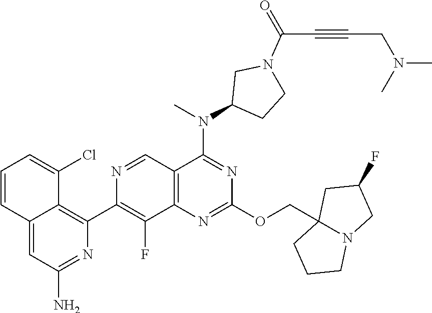 [SMILES: CN(C)CC#CC(=O)N1CC[C@@H](N(C)c2nc(OCC34CCCN3C[C@H](F)C4)nc3c(F)c(-c4nc(N)cc5cccc(Cl)c45)ncc23)C1] | ¹H NMR (400 MHz, CD₃CN) δ 9.19-9.13 (m, 1H), 7.67-7.60 (m, 1H), 7.47-7.37 (m, 1H), 7.28-7.20 (m, 1H), 6.95-6.89 (m, 1H), 5.19 (s, 2H), 5.15 (s, 2H), 4.25-4.08 (m, 2H), 4.03-3.85 (m, 1H), 3.79-3.64 (m, 2H), 3.52-3.36 (m, 6H), 3.20-3.08 (m, 2H), 3.08- 3.04 (m, 1H), 2.94-2.84 (m, 1H), 2.40- 2.30 (m, 2H), 2.28 (s, 3H), 2.25-2.22 (m, 3H), 2.12-2.00 (m, 3H), 1.91-1.80 (m, 3H) | 690.3 2.665 min |
| 92 | 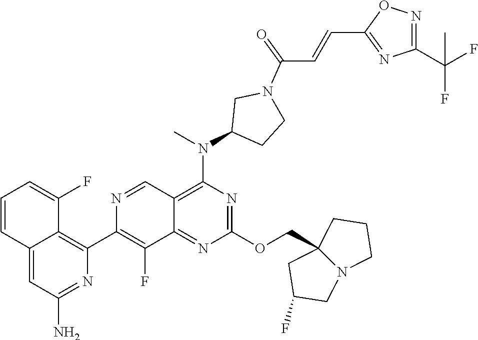 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1] | ¹H NMR (400 MHz, CD₃CN) δ 9.28-9.21 (m, 1H), 7.67-7.42 (m, 4H), 6.96-6.80 (m, 2H), 5.48-5.33 (m, 2H), 5.24-5.13 (m, 2H), 4.30-4.17 (m, 3H), 4.05 (s, 1H), 3.95-3.76 (m, 2H), 3.69-3.47 (m, 2H), 3.46 (s, 3H), 3.27-3.09 (m, 4H), 2.99-2.90 (m, 1H), 2.45 (d, J = 8.3 Hz, 1H), 2.35 (d, J = 8.3 Hz, 1H), 2.11 (s, 2H), 2.07 (d, J = 7.9 Hz, 2H), 1.91 (dd, J = 5.3, 11.5 Hz, 2H) | 751.2 2.185 min |
| 93 | 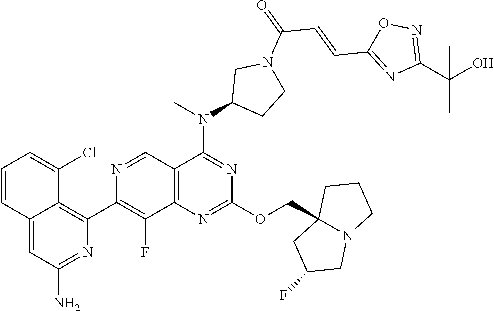 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1] | ¹H NMR (400 MHz, CD₃CN) δ 9.22-9.13 (m, 1H), 7.67 (d, J = 4.0 Hz, 1H), 7.52-7.32 (m, 3H), 7.26-7.20 (m, 1H), 6.93-6.89 (m, 1H), 5.46-5.33 (m, 1H), 5.32 (s, 1H), 5.17 (s, 2H), 4.25-4.10 (m, 3H), 4.05-3.95 (m, 1H), 3.91-3.82 (m, 1H), 3.80-3.70 (m, 1H), 3.46 (s, 2H), 3.44-3.38 (m, 3H), 3.21-3.07 (m, 2H), 2.94-2.81 (m, 1H), 2.46-2.37 (m, 1H), 2.36-2.28 (m, 1H), 2.12-2.00 (m, 3H), 1.91-1.78 (m, 3H), 1.57 (d, J = 9.1 Hz, 6H) | 761.3 2.697 min |
| 97 | 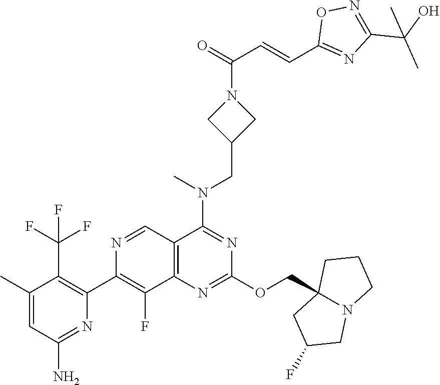 [SMILES: Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F] | ¹H NMR (400 MHz, CD₃CN) δ 9.21 (s, 1H), 7.39-7.29 (m, 1H), 7.22-7.14 (m, 1H), 6.59 (s, 1H), 5.48 (s, 2H), 5.37-5.19 (m, 1H), 4.48 (t, J = 8.6 Hz, 1H), 4.27 (dd, J = 5.4, 14.2 Hz, 2H), 4.22-4.13 (m, 4H), 3.95 (dd, J = 5.6, 10.6 Hz, 1H), 3.57 (s, 3H), 3.18 (d, J = 13.0 Hz, 2H), 3.14-3.03 (m, 2H), 2.95-2.88 (m, 1H), 2.46 (d, J = 1.6 Hz, 3H), 2.13-2.05 (m, 3H), 1.93-1.85 (m, 3H), 1.59 (s, 6H) | 759.3 2.526 min |
| 98 | 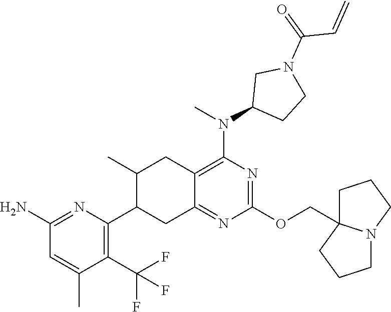 [SMILES: C=CC(=O)N1CC[C@@H](N(C)c2nc(OCC34CCCN3CCC4)nc3c2CC(C)C(c2nc(N)cc(C)c2C(F)(F)F)C3)C1] | ¹H NMR (400 MHz, CD₃CN) δ 6.66-6.45 (m, 1H), 6.33-6.14 (m, 2H), 5.78-5.54 (m, 1H), 5.23 (s, 2H), 4.79-4.47 (m, 1H), 4.11- 3.85 (m, 3H), 3.84-3.69 (m, 1H), 3.66-3.29 (m, 2H), 3.24-3.12 (m, 1H), 3.07-3.00 (m, 1H), 2.96 (dd, J = 5.1, 10.1 Hz, 2H), 2.93- 2.89 (m, 3H), 2.88-2.80 (m, 1H), 2.72-2.55 (m, 3H), 2.54-2.43 (m, 1H), 2.40-2.34 (m, 3H), 2.19-2.02 (m, 3H), 1.95-1.87 (m, 2H), 1.87-1.71 (m, 4H), 1.65-1.52 (m, 2H), 0.77 (d, J = 6.4 Hz, 3H) | 614.4 2.603/2.643 min |
| 99 | 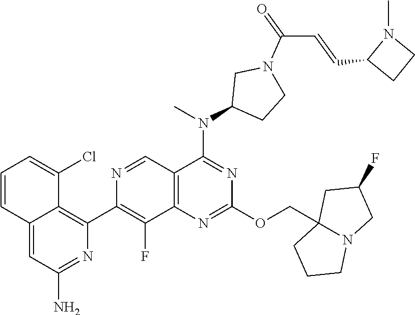 [SMILES: CN1CC[C@@H]1/C=C/C(=O)N1CC[C@@H](N(C)c2nc(OCC34CCCN3C[C@H](F)C4)nc3c(F)c(-c4nc(N)cc5cccc(Cl)c45)ncc23)C1] | ¹H NMR (400 MHz, CD₃CN) δ 9.21-9.10 (m, 1H), 7.71-7.59 (m, 1H), 7.41 (t, J = 7.9 Hz, 1H), 7.25-7.14 (m, 1H), 6.96-6.83 (m, 1H), 6.80-6.69 (m, 1H), 6.45-6.30 (m, 1H), 5.45-5.30 (m, 2H), 5.17-5.29 (m, 2H), 4.57- 4.43 (m, 1H), 4.26-4.16 (m, 1H), 4.16-4.09 (m, 1H), 4.08 (d, J = 2.3 Hz, 1H), 3.97-3.71 (m, 2H), 3.69-3.47 (m, 3H), 3.41-3.37 (m, 3H), 3.29-3.22 (m, 1H), 3.13 (d, J = 8.3 Hz, 2H), 3.08-3.04 (m, 1H), 2.90-2.85 (m, 1H), 2.28-2.20 (m, 2H), 2.19-2.01 (m, 5H), 1.92- 1.75 (m, 5H) | 704.4 2.704 min |
| 100 | 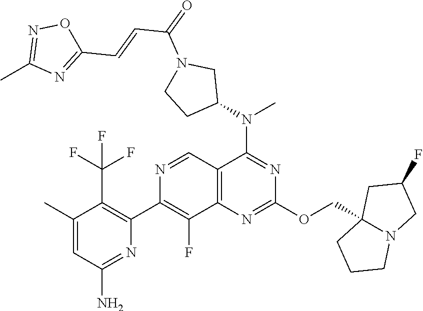 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1] | ¹H NMR (400 MHz, CD₃CN) δ 9.16 (d, J = 1.9 Hz, 1H), 7.57-7.32 (m, 2H), 6.59 (s, 1H), 5.50-5.41 (m, 2H), 5.35-5.18 (m, 1H), 4.26- 4.12 (m, 3H), 4.07-3.98 (m, 1H), 3.83- 3.72 (m, 1H), 3.67-3.48 (m, 1H), 3.42 (s, 3H), 3.19-3.06 (m, 3H), 2.98-2.82 (m, 1H), 2.46 (d, J = 1.6 Hz, 3H), 2.41 (d, J = 8.4 Hz, 3H), 2.37-2.30 (m, 1H), 2.21 (d, J = 4.5 Hz, 2H), 2.14-2.04 (m, 3H), 1.94-1.82 (m, 3H) | 715.3 2.631 min |
| 101 | 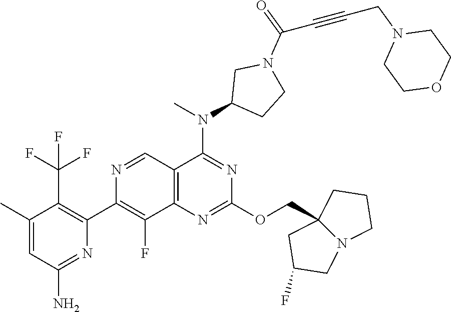 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F] | ¹H NMR (400 MHz, CD₃CN) δ 9.20 (d, J = 3.4 Hz, 1H), 6.60 (s, 1H), 5.54-5.46 (m, 2H), 5.44-5.38 (m, 1H), 4.63-4.52 (m, 1H), 4.19- 3.96 (m, 1H), 3.94-3.79 (m, 1H), 3.77- 3.72 (m, 1H), 3.70-3.62 (m, 9H), 3.52 (s, 1H), 3.50 (s, 1H), 3.46 (s, 1H), 3.44 (s, 1H), 3.28-3.14 (m, 1H), 2.54 (td, J = 4.6, 15.1 Hz, 4H), 2.47 (d, J = 1.4 Hz, 3H), 2.41-2.33 (m, 2H), 2.01-1.99 (m, 2H), 1.87-1.79 (m, 6H) | 730.2 1.865 min |
| 102 | 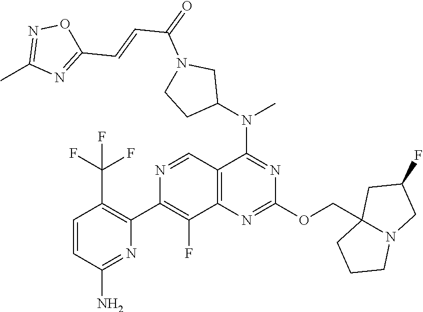 [SMILES: Cc1noc(/C=C/C(=O)N2CCC(N(C)c3nc(OCC45CCCN4C[C@H](F)C5)nc4c(F)c(-c5nc(N)ccc5C(F)(F)F)ncc34)C2)n1] | ¹H NMR (400 MHz, CD₃CN) δ 9.17 (d, J = 1.3 Hz, 1H), 7.84 (d, J = 8.9 Hz, 1H), 7.53- 7.32 (m, 2H), 6.73 (d, J = 8.8 Hz, 1H), 5.62 (s, 2H), 5.47-5.14 (m, 2H), 4.26-4.19 (m, 1H), 4.18-4.13 (m, 1H), 4.02 (dd, J = 8.0, 12.8 Hz, 1H), 3.95-3.70 (m, 2H), 3.66-3.47 (m, 1H), 3.42 (s, 3H), 3.23-3.11 (m, 2H), 3.10-3.03 (m, 1H), 2.98-2.84 (m, 1H), 2.46- 2.39 (m, 4H), 2.37-2.30 (m, 1H), 2.25 (s, 1H), 2.14-2.06 (m, 2H), 1.94-1.79 (m, 3H) | 701.3 2.530 min |
| 103 | 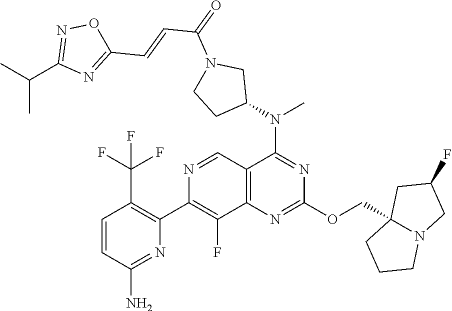 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1] | ¹H NMR (400 MHz, CD₃CN) δ 9.18 (d, J = 2.3 Hz, 1H), 7.84 (d, J = 8.9 Hz, 1H), 7.52- 7.32 (m, 2H), 6.73 (d, J = 8.8 Hz, 1H), 5.60 (s, 2H), 5.47-5.17 (m, 2H), 4.27-4.12 (m, 2H), 4.02-3.85 (m, 2H), 3.82-3.74 (m, 1H), 3.66-3.49 (m, 1H), 3.43 (d, J = 1.0 Hz, 3H), 3.22-3.07 (m, 4H), 2.97-2.87 (m, 1H), 2.48- 2.40 (m, 1H), 2.38-2.29 (m, 1H), 2.27- 2.21 (m, 1H), 2.13 (s, 1H), 2.07 (d, J = 5.3 Hz, 1H), 1.93-1.78 (m, 3H), 1.35 (dd, J = 7.0, 8.0 Hz, 6H) | 729.3 2.743 min |
| 104 | 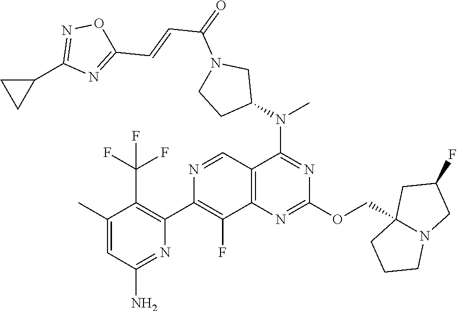 [SMILES: Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F] | ¹H NMR (400 MHz, CD₃CN) δ 9.18-9.13 (m, 1H), 7.48-7.28 (m, 2H), 6.59 (s, 1H), 5.47 (s, 2H), 5.34-5.16 (m, 1H), 4.26-4.20 (m, 1H), 4.19-4.13 (m, 1H), 4.05-3.96 (m, 1H), 3.92-3.85 (m, 1H), 3.65-3.51 (m, 1H), 3.42 (s, 3H), 3.20-3.07 (m, 3H), 2.95- 2.87 (m, 1H), 2.46 (s, 3H), 2.35-2.31 (m, 1H), 2.21 (s, 2H), 2.13-2.07 (m, 4H), 1.93- 1.83 (m, 4H), 1.14-1.09 (m, 2H), 1.03-0.97 (m, 2H) | 741.3 2.770 min |
| 105 | 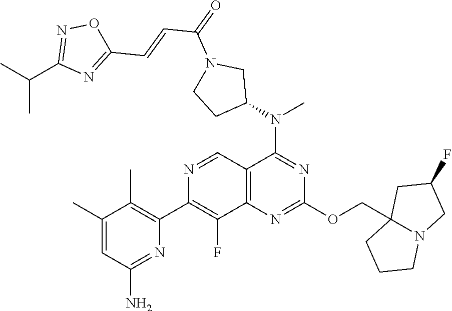 [SMILES: Cc1cc(N)nc(-c2ncc3c(N(C)[C@@H]4CCN(C(=O)/C=C/c5nc(C(C)C)no5)C4)nc(OCC45CCCN4C[C@H](F)C5)nc3c2F)c1C] | ¹H NMR (400 MHz, CD₃CN) δ 9.23-9.13 (m, 1H), 7.39 (d, J = 11.5 Hz, 2H), 6.48 (s, 1H), 5.44-5.15 (m, 2H), 4.70 (s, 2H), 4.24- 4.18 (m, 1H), 4.17-4.10 (m, 1H), 4.05-3.95 (m, 1H), 3.91-3.82 (m, 1H), 3.81-3.70 (m, 1H), 3.64-3.46 (m, 1H), 3.41 (s, 3H), 3.20- 3.08 (m, 3H), 3.06 (d, J = 5.9 Hz, 1H), 2.88 (s, 1H), 2.45-2.37 (m, 1H), 2.35-2.28 (m, 1H), 2.25 (s, 3H), 2.18 (s, 2H), 2.10 (d, J = 2.4 Hz, 4H), 1.90-1.76 (m, 3H), 1.37-1.26 (m, 6H). | 689.4 2.843 min |
| 106 | 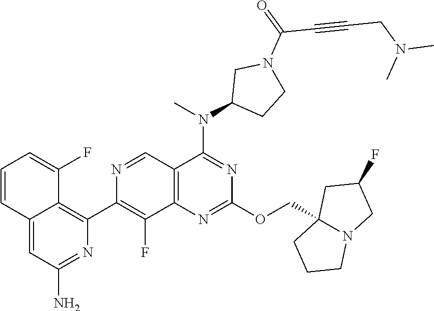 [SMILES: CN(C)CC#CC(=O)N1CC[C@@H](N(C)c2nc(OC[C@@]34CCCN3C[C@H](F)C4)nc3c(F)c(-c4nc(N)cc5cccc(F)c45)ncc23)C1] | ¹H NMR (400 MHz, CD₃CN) δ 9.21 (d, J = 4.4 Hz, 1H), 7.52-7.46 (m, 2H), 6.94-6.81 (m, 2H), 5.36 (s, 1H), 5.26-5.18 (m, 2H), 4.30-4.12 (m, 3H), 4.05-3.87 (m, 1H), 3.81- 3.65 (m, 2H), 3.53-3.46 (m, 2H), 3.43 (d, J = 6.9 Hz, 4H), 3.22-3.15 (m, 2H), 3.14- 3.09 (m, 1H), 2.97-2.89 (m, 1H), 2.31 (s, 6H), 2.25-2.19 (m, 2H), 2.16-2.05 (m, 3H), 1.93-1.84 (m, 3H) | 674.2 1.811 min |
| 108 | 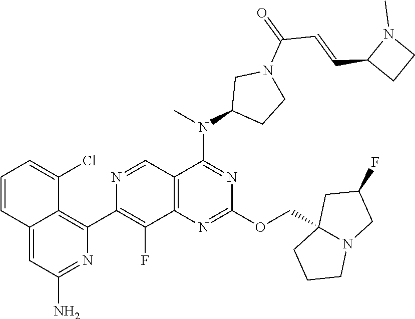 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | ¹H NMR (400 MHz, CD₃CN) δ 9.23-9.12 (m, 1H), 7.68-7.58 (m, 1H), 7.46-7.35 (m, 1H), 7.27-7.20 (m, 1H), 6.95-6.90 (m, 1H), 6.81-6.71 (m, 1H), 6.42-6.31 (m, 1H), 5.19 (s, 2H), 5.16 (s, 2H), 4.24-4.17 (m, 1H), 4.16-4.09 (m, 1H), 4.08-3.75 (m, 2H), 3.69- 3.60 (m, 1H), 3.58-3.43 (m, 2H), 3.42- 3.38 (m, 3H), 3.32-3.23 (m, 1H), 3.19-3.08 (m, 2H), 3.08-3.04 (m, 1H), 2.94-2.85 (m, 1H), 2.84-2.75 (m, 1H), 2.41-2.28 (m, 2H), 2.23 (d, J = 9.4 Hz, 3H), 2.12-1.99 (m, 4H), 1.92-1.79 (m, 4H) | 704.4 2.677 min |
| 109 | 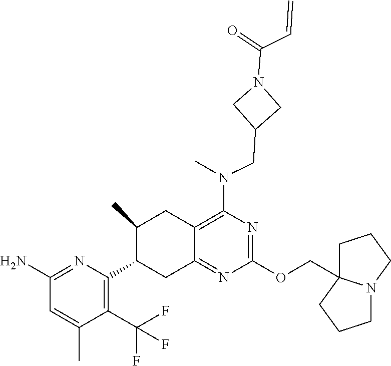 [SMILES: C=CC(=O)N1CC(CN(C)c2nc(OCC34CCCN3CCC4)nc3c2C[C@H](C)[C@@H](c2nc(N)cc(C)c2C(F)(F)F)C3)C1] | ¹H NMR (400 MHz, CD₃CN) δ 6.35-6.22 (m, 2H), 6.19-6.12 (m, 1H), 5.66-5.59 (m, 1H), 5.22 (s, 2H), 4.28 (q, J = 8.8 Hz, 1H), 4.09-3.83 (m, 5H), 3.79-3.57 (m, 1H), 3.51- 3.37 (m, 1H), 3.17 (dt, J = 6.3, 10.4 Hz, 1H), 3.02 (s, 3H), 3.01-2.92 (m, 3H), 2.87- 2.77 (m, 1H), 2.70-2.55 (m, 3H), 2.53-2.43 (m, 1H), 2.38 (q, J = 3.5 Hz, 3H), 2.17-2.03 (m, 2H), 1.95-1.86 (m, 2H), 1.85-1.69 (m, 4H), 1.63-1.53 (m, 2H), 0.76 (d, J = 6.4 Hz, 3H) | 614.4 2.709 min |
| 110 | 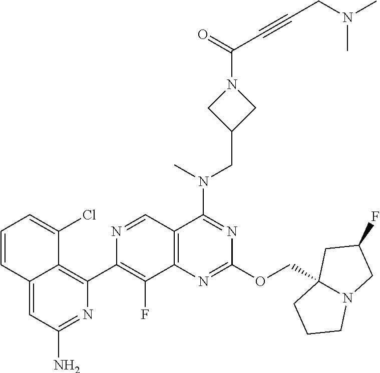 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1] | ¹H NMR (400 MHz, CD₃CN) δ 8.92 (dd, J = 1.9, 5.2 Hz, 1H), 7.62 (d, J = 8.4 Hz, 1H), 7.44-7.36 (m, 1H), 7.23 (d, J = 7.3 Hz, 1H), 6.87 (s, 1H), 5.36-5.10 (m, 3H), 4.23-4.02 (m, 3H), 3.99-3.80 (m, 2H), 3.77-3.63 (m, 2H), 3.55-3.36 (m, 3H), 3.30-3.22 (m, 2H), 3.14-3.04 (m, 3H), 2.96-2.87 (m, 3H), 2.16 (d, J = 1.3 Hz, 6H), 2.11-2.00 (m, 3H), 1.90- 1.76 (m, 3H) | 690.3 2.474 min |
| 111 | 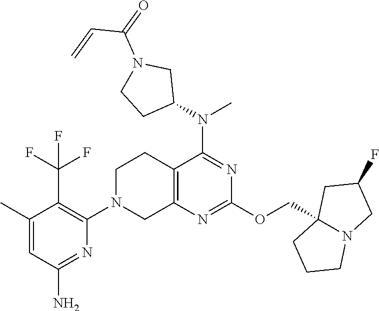 [SMILES: C=CC(=O)N1CC[C@@H](N(C)c2nc(OC[C@@]34CCCN3C[C@H](F)C4)nc3c2CCN(c2nc(N)cc(C)c2C(F)(F)F)C3)C1] | ¹H NMR (400 MHz, CD₃CN) δ 6.62-6.44 (m, 1H), 6.27-6.13 (m, 1H), 6.01 (s, 1H), 5.62 (ddd, J = 2.4, 7.6, 10.1 Hz, 1H), 5.31- 5.25 (m, 1H), 5.19-5.11 (m, 3H), 4.80-4.67 (m, 1H), 4.19 (s, 2H), 4.01-3.98 (m, 1H), 3.94-3.88 (m, 2H), 3.83-3.76 (m, 1H), 3.59- 3.49 (m, 1H), 3.40-3.34 (m, 2H), 3.26 (d, J = 1.8 Hz, 1H), 3.09 (s, 2H), 3.03-3.00 (m, 1H), 2.91 (d, J = 2.8 Hz, 3H), 2.87-2.81 (m, 2H), 2.73-2.69 (m, 1H), 2.28 (d, J = 2.0 Hz, 3H), 2.18 (s, 1H), 2.04 (d, J = 3.1 Hz, 2H), 1.81 (dd, J = 7.5, 13.4 Hz, 4H) | 619.4 2.941 min |
| 112 | 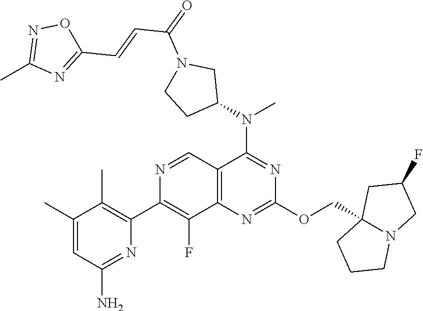 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1] | ¹H NMR (400 MHz, CD₃CN) δ 9.23-9.14 (m, 1H), 7.51-7.31 (m, 2H), 6.54-6.43 (m, 1H), 5.44-5.14 (m, 2H), 4.77-4.64 (m, 2H), 4.23-4.17 (m, 1H), 4.13 (d, J = 2.8 Hz, 1H), 4.04-3.96 (m, 1H), 3.91-3.82 (m, 1H), 3.80- 3.70 (m, 1H), 3.64-3.45 (m, 1H), 3.40 (s, 3H), 3.18-3.08 (m, 2H), 3.07-3.03 (m, 1H), 2.93-2.83 (m, 1H), 2.44-2.40 (m, 1H), 2.38 (d, J = 8.1 Hz, 3H), 2.35-2.28 (m, 1H), 2.25 (s, 3H), 2.18 (d, J = 4.4 Hz, 2H), 2.12-1.99 (m, 4H), 1.90-1.76 (m, 3H). | 661.4 2.626 min |
| 113 | 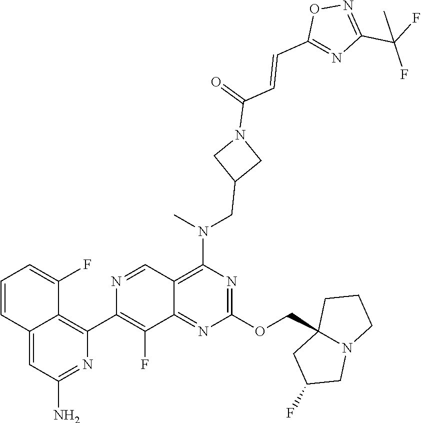 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12] | ¹H NMR (400 MHz, CD₃CN) δ 9.30-9.17 (m, 1H), 7.50-7.44 (m, 2H), 7.41-7.35 (m, 1H), 7.30-7.22 (m, 1H), 6.92-6.87 (m, 1H), 6.86-6.78 (m, 1H), 5.37-5.19 (m, 1H), 5.19- 5.10 (m, 2H), 4.53-4.43 (m, 1H), 4.33- 4.08 (m, 6H), 3.99-3.90 (m, 1H), 3.61-3.52 (m, 3H), 3.28-3.20 (m, 1H), 3.15 (d, J = 13.6 Hz, 2H), 3.09-3.04 (m, 1H), 2.95-2.83 (m, 1H), 2.23-2.17 (m, 3H), 2.13 (s, 1H), 2.08 (s, 1H), 2.03 (s, 1H), 1.90-1.75 (m, 3H) | 751.3 2.904 min |
| 114 | 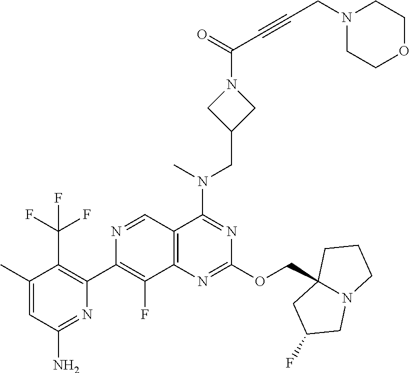 [SMILES: Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F] | ¹H NMR (400 MHz, CD₃CN) δ 9.19-9.14 (m, 1H), 6.56 (s, 1H), 5.44 (s, 2H), 5.35- 5.18 (m, 1H), 4.28 (t, J = 8.7 Hz, 1H), 4.22- 4.00 (m, 6H), 3.82 (dd, J = 5.6, 9.9 Hz, 1H), 3.64-3.58 (m, 4H), 3.53 (s, 3H), 3.43 (s, 2H), 3.23-3.11 (m, 3H), 3.06 (s, 1H), 2.93- 2.85 (m, 1H), 2.49-2.45 (m, 4H), 2.43 (d, J = 1.4 Hz, 3H), 2.20-2.15 (m, 1H), 2.11 (s, 2H), 1.91-1.79 (m, 3H) | 730.4 2.612 min |
| 115 | 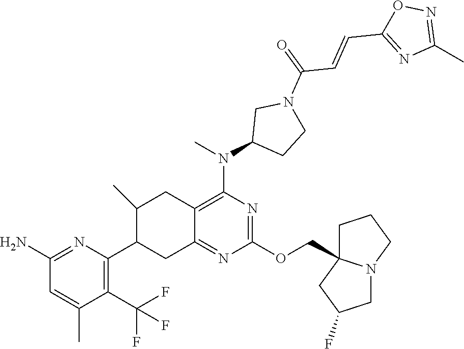 [SMILES: Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1] | ¹H NMR (400 MHz, CD₃CN) δ 7.54-7.33 (m, 2H), 6.29 (s, 1H), 5.36-5.16 (m, 3H), 4.80-4.59 (m, 1H), 4.12-3.92 (m, 3H), 3.88- 3.78 (m, 1H), 3.77-3.60 (m, 1H), 3.53- 3.40 (m, 1H), 3.32 (s, 1H), 3.24-3.12 (m, 3H), 3.09-2.99 (m, 2H), 2.94 (d, J = 4.9 Hz, 3H), 2.89 (d, J = 5.1 Hz, 2H), 2.74-2.62 (m, 1H), 2.58-2.48 (m, 1H), 2.43 (s, 1H), 2.41 (d, J = 2.4 Hz, 1H), 2.39 (d, J = 3.4 Hz, 2H), 2.31 (s, 2H), 2.18 (d, J = 4.8 Hz, 2H), 2.09 (s, 2H), 2.06-2.01 (m, 1H), 1.94-1.76 (m, 3H), 0.79 (d, J = 6.2 Hz, 3H) | 714.3 2.971/3.009 min |
| 116 | 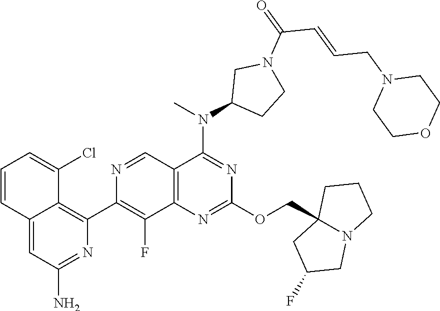 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/CN2CCOCC2)C1] | ¹H NMR (400 MHz, CD₃CN) δ 9.21-9.14 (m, 1H), 7.63 (d, J = 8.4 Hz, 1H), 7.41 (t, J = 7.9 Hz, 1H), 7.28-7.18 (m, 1H), 6.95-6.89 (m, 1H), 6.80-6.64 (m, 1H), 6.48-6.33 (m, 1H), 5.44-5.17 (m, 2H), 5.17-5.10 (m, 2H), 4.24-4.17 (m, 1H), 4.16-4.08 (m, 1H), 4.08- 3.89 (m, 1H), 3.88-3.76 (m, 1H), 3.68- 3.58 (m, 5H), 3.57-3.43 (m, 1H), 3.40 (s, 3H), 3.20-3.01 (m, 5H), 2.94-2.84 (m, 1H), 2.40 (s, 4H), 2.32-2.17 (m, 2H), 2.12-2.02 (m, 3H), 1.92-1.75 (m, 3H) | 734.4 2.418 min |
| 117 | 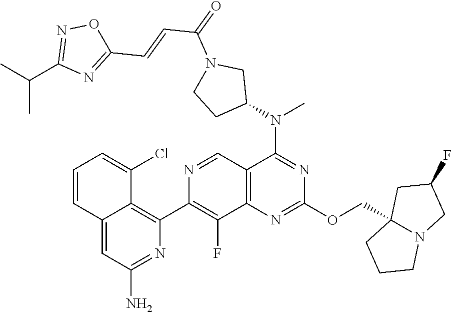 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1] | ¹H NMR (400 MHz, CD₃CN) δ 9.18-9.13 (m, 1H), 7.64-7.60 (m, 1H), 7.47-7.31 (m, 3H), 7.24-7.19 (m, 1H), 6.90 (s, 1H), 5.45- 5.24 (m, 2H), 5.18-5.13 (m, 2H), 4.27-4.06 (m, 3H), 3.98 (d, J = 7.8 Hz, 1H), 3.92-3.67 (m, 2H), 3.64-3.47 (m, 1H), 3.41 (s, 3H), 3.16-3.06 (m, 3H), 2.92-2.82 (m, 1H), 2.44- 2.20 (m, 3H), 2.04 (d, J = 4.3 Hz, 2H), 1.88- 1.72 (m, 3H), 1.31 (dd, J = 7.0, 8.4 Hz, 6H) | 745.3 2.805 min |
| 118 | 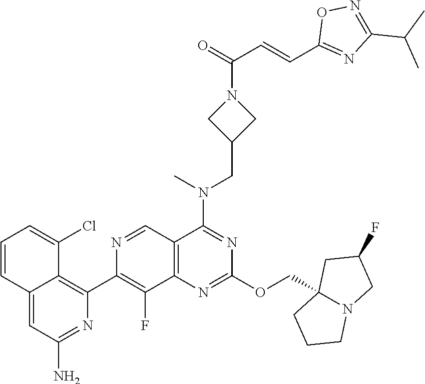 [SMILES: CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1] | ¹H NMR (400 MHz, CDCl₃) δ 9.22-9.17 (m, 1H), 7.57-7.47 (m, 2H), 7.37 (t, J = 7.9 Hz, 1H), 7.25 (d, J = 7.3 Hz, 1H), 7.10 (d, J = 15.6 Hz, 1H), 6.88 (s, 1H), 5.38-5.19 (m, 1H), 4.65 (s, 2H), 4.52 (s, 1H), 4.37-4.01 (m, 7H), 3.61 (s, 3H), 3.30-3.21 (m, 3H), 3.15 (d, J = 6.9 Hz, 2H), 2.98 (s, 1H), 2.28- 2.11 (m, 3H), 1.93 (s, 3H), 1.37 (d, J = 7.0 Hz, 6H). | 745.3 2.759 min |
| 119 | 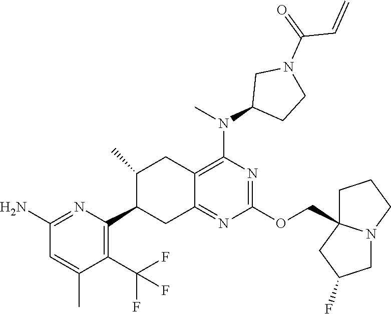 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1] | ¹H NMR (400 MHz, CD₃CN) δ 6.60 (dd, J = 10.4, 16.8 Hz, 1H), 6.31-6.18 (m, 2H), 5.72- 5.61 (m, 1H), 5.35-5.14 (m, 3H), 4.76- 4.55 (m, 1H), 4.07-3.89 (m, 3H), 3.85-3.71 (m, 1H), 3.62-3.50 (m, 1H), 3.44-3.31 (m, 1H), 3.22-3.11 (m, 3H), 3.07 (s, 1H), 3.03- 2.97 (m, 1H), 2.95-2.80 (m, 5H), 2.68-2.60 (m, 1H), 2.55-2.45 (m, 1H), 2.38 (d, J = 3.5 Hz, 3H), 2.28 (s, 1H), 2.15 (s, 1H), 2.08 (s, 2H), 2.04 (dd, J = 2.8, 9.6 Hz, 2H), 1.93- 1.77 (m, 3H), 0.77 (d, J = 6.4 Hz, 3H) | 632.3 2.864 min |
| 120 | 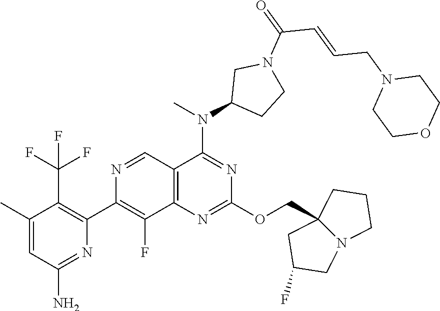 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F] | ¹H NMR (400 MHz, CD₃CN) δ 9.10 (d, J = 3.1 Hz, 1H), 6.70 (dtd, J = 3.1, 6.1, 15.2 Hz, 1H), 6.54 (s, 1H), 6.44-6.29 (m, 1H), 5.44 (s, 2H), 5.38-5.14 (m, 1H), 5.16 (s, 1H), 4.22-4.14 (m, 1H), 4.12-4.06 (m, 1H), 4.06- 3.85 (m, 1H), 3.85-3.71 (m, 1H), 3.65- 3.55 (m, 5H), 3.49 (dd, J = 7.8, 12.6 Hz, 1H), 3.44-3.37 (m, 1H), 3.35 (d, J = 2.5 Hz, 3H), 3.12-3.02 (m, 4H), 2.91-2.82 (m, 1H), 2.42- 2.36 (m, 6H), 2.27-2.18 (m, 1H), 2.16 (s, 1H), 2.09-1.98 (m, 2H), 1.90-1.77 (m, 3H) | 732.4 0.928 min |
| 121 | 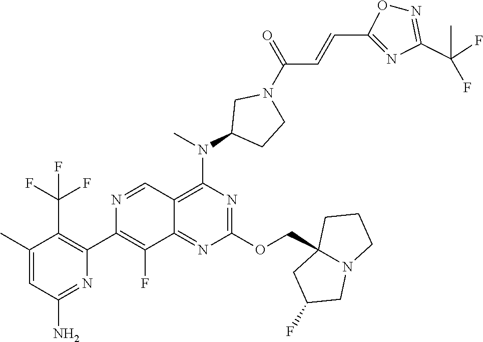 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F] | ¹H NMR (400 MHz, CD₃CN) δ 9.06 (s, 1H), 7.62-7.24 (m, 2H), 6.49 (s, 1H), 5.43- 5.28 (m, 2H), 5.27-5.09 (m, 1H), 4.19-4.02 (m, 3H), 3.93 (dd, J = 7.8, 13.1 Hz, 1H), 3.87- 3.75 (m, 1H), 3.74-3.63 (m, 1H), 3.59- 3.37 (m, 1H), 3.33 (s, 3H), 3.13-2.95 (m, 3H), 2.87-2.76 (m, 1H), 2.35-2.29 (m, 1H), 2.25 (t, J = 8.4 Hz, 1H), 2.18-2.05 (m, 5H), 2.04-1.96 (m, 4H), 1.84-1.72 (m, 3H) | 765.3 2.842 min |
| 122 | 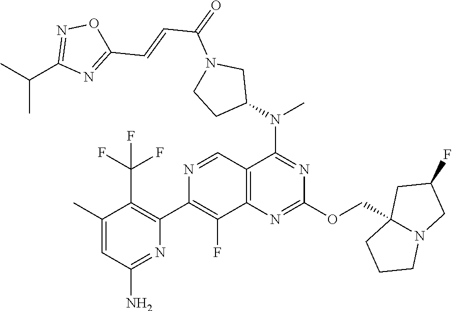 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F] | ¹H NMR (400 MHz, CD₃CN) δ 9.18-9.10 (m, 1H), 7.52-7.31 (m, 2H), 6.62-6.56 (m, 1H), 5.48 (s, 2H), 5.42-5.18 (m, 2H), 4.27- 4.11 (m, 3H), 4.02 (dd, J = 8.0, 12.5 Hz, 1H), 3.93-3.72 (m, 2H), 3.65-3.49 (m, 1H), 3.45- 3.37 (m, 3H), 3.20-3.05 (m, 4H), 2.96- 2.85 (m, 1H), 2.51-2.42 (m, 4H), 2.38-2.26 (m, 2H), 2.13-2.08 (m, 2H), 1.93-1.81 (m, 3H), 1.35 (dd, J = 7.0, 8.3 Hz, 6H) | 743.3 2.834 min |
| 123 | 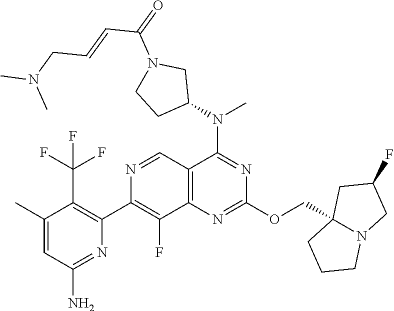 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F] | ¹H NMR (400 MHz, CD₃CN) δ 9.15 (d, J = 2.8 Hz, 1H), 6.81-6.68 (m, 1H), 6.59 (s, 1H), 6.45-6.33 (m, 1H), 5.48-5.35 (m, 2H), 5.34- 5.20 (m, 1H), 4.27-4.19 (m, 1H), 4.18- 4.11 (m, 1H), 4.05-3.84 (m, 1H), 3.69-3.43 (m, 2H), 3.41 (d, J = 2.8 Hz, 3H), 3.21-3.12 (m, 2H), 3.11-3.04 (m, 3H), 2.96-2.87 (m, 1H), 2.46 (d, J = 1.3 Hz, 3H), 2.42-2.27 (m, 2H), 2.22 (d, J = 6.5 Hz, 6H), 2.17-2.05 (m, 5H), 1.92-1.84 (m, 3H) | 690.4 2.489 min |
| 124 | 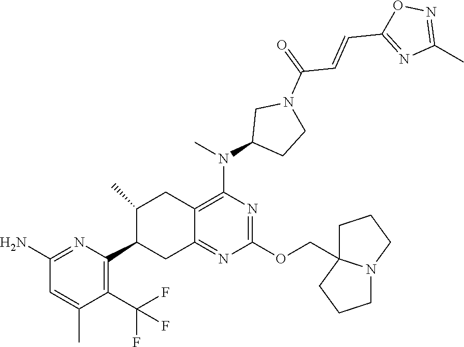 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1] | ¹H NMR (400 MHz, CD₃CN) δ 7.51-7.43 (m, 1H), 7.41-7.33 (m, 1H), 6.28 (s, 1H), 5.24 (s, 2H), 4.79-4.58 (m, 1H), 4.16 (dd, J = 8.0, 10.3 Hz, 1H), 4.03-3.89 (m, 3H), 3.87- 3.74 (m, 1H), 3.68 (d, J = 8.6 Hz, 1H), 3.52 3.36 (m, 1H), 3.17 (td, J = 5.3, 10.3 Hz, 1H), 3.07-2.94 (m, 3H), 2.93 (d, J = 3.1 Hz, 3H), 2.89-2.81 (m, 1H), 2.66 (s, 4H), 2.41 (s, 1H), 2.40 (s, 1H), 2.38 (d, J = 3.5 Hz, 2H), 2.30-2.23 (m, 1H), 2.21-2.14 (m, 1H), 2.13- 2.03 (m, 2H), 1.91 (td, J = 6.1, 12.1 Hz, 2H), 1.86-1.71 (m, 4H), 1.66-1.54 (m, 2H), 0.77 (d, J = 6.3 Hz, 3H) | 696.4 2.987 min |
| 129 | 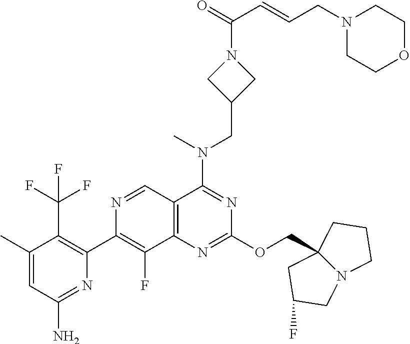 [SMILES: Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F] | ¹H NMR (400 MHz, CD₃CN) δ 9.20-9.12 (m, 1H), 6.68-6.53 (m, 2H), 6.09 (d, J = 15.0) Hz, 1H), 5.45 (s, 2H), 5.35-5.16 (m, 1H), 4.29 (s, 1H), 4.18-4.05 (m, 6H), 3.83-3.78 (m, 1H), 3.63-3.58 (m, 4H), 3.53 (s, 3H), 3.18-3.12 (m, 3H), 3.07 (d, J = 4.9 Hz, 3H), 2.92-2.87 (m, 1H), 2.43 (s, 4H), 2.37 (s, 3H), 2.04 (s, 3H), 1.89-1.73 (m, 3H) | 732.3 2.433 min |
| 130 | 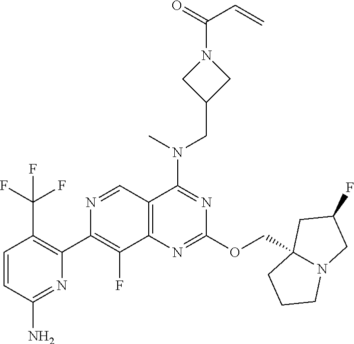 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1] | ¹H NMR (400 MHz, CD₃CN) δ 9.22 (s, 1H), 7.84 (d, J = 8.8 Hz, 1H), 6.73 (d, J = 8.8 Hz, 1H), 6.37-6.25 (m, 1H), 6.22-6.13 (m, 1H), 5.69-5.55 (m, 3H), 5.39-5.18 (m, 1H), 4.35 (t, J = 8.4 Hz, 1H), 4.24-4.08 (m, 6H), 3.86 (dd, J = 5.6, 9.8 Hz, 1H), 3.56 (s, 3H), 3.22- 3.07 (m, 4H), 2.97-2.88 (m, 1H), 2.12 (s, 2H), 2.06 (d, J = 6.8 Hz, 1H), 1.92-1.77 (m, 3H) | 619.3 2.580 min |
| 132 | 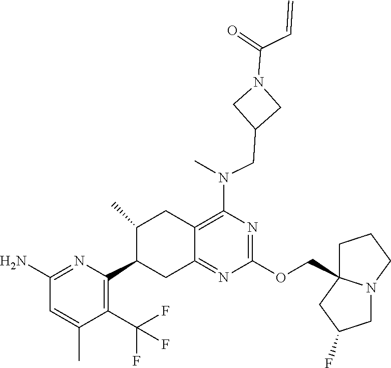 [SMILES: C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1] | ¹H NMR (400 MHz, CD₃CN) δ 6.35-6.22 (m, 2H), 6.20-6.12 (m, 1H), 5.67-5.59 (m, 1H), 5.35-5.15 (m, 3H), 4.28 (q, J = 8.5 Hz, 1H), 4.08-3.97 (m, 3H), 3.96-3.84 (m, 2H), 3.80-3.58 (m, 1H), 3.45 (ddd, J = 7.0, 13.9, 17.2 Hz, 1H), 3.22-3.10 (m, 3H), 3.08-2.94 (m, 6H), 2.93-2.78 (m, 2H), 2.66 (dd, J = 4.4, 15.8 Hz, 1H), 2.53-2.44 (m, 1H), 2.38 (q, J = 3.3 Hz, 3H), 2.15-2.06 (m, 3H), 2.04- 2.00 (m, 1H), 1.93-1.77 (m, 3H), 0.82- 0.67 (m, 3H). | 632.3 2.855 min |
| 134 | 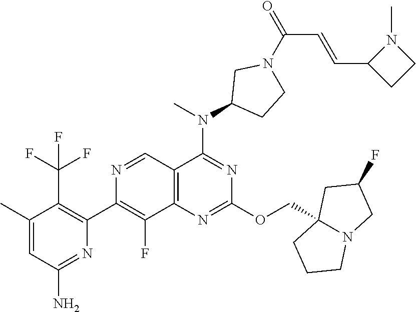 [SMILES: Cc1cc(N)nc(-c2ncc3c(N(C)[C@@H]4CCN(C(=O)/C=C/C5CCN5C)C4)nc(OC[C@@]45CCCN4C[C@H](F)C5)nc3c2F)c1C(F)(F)F] | ¹H NMR (400 MHz, CD₃CN) δ 9.14 (d, J = 2.1 Hz, 1H), 6.78 (ddd, J = 1.8, 5.7, 15.1 Hz, 1H), 6.58 (s, 1H), 6.39 (t, J = 14.6 Hz, 1H), 5.50 (s, 2H), 5.41-5.18 (m, 2H), 4.28-4.19 (m, 1H), 4.18-4.10 (m, 1H), 4.10-3.89 (m, 1H), 3.89-3.73 (m, 1H), 3.70-3.61 (m, 1H), 3.56 (dd, J = 4.8, 12.0 Hz, 1H), 3.40 (d, J = 1.5 Hz, 3H), 3.34-3.27 (m, 1H), 3.16 (d, J = 7.6 Hz, 2H), 3.09 (s, 1H), 2.97-2.87 (m, 1H), 2.87-2.77 (m, 1H), 2.46 (d, J = 1.5 Hz, 3H), 2.41-2.34 (m, 1H), 2.27 (s, 3H), 2.21-2.11 (m, 4H), 2.10-2.03 (m, 1H), 1.99 (s, 1H), 1.95-1.80 (m, 4H) | 702.4 2.686 min |
| 135 | 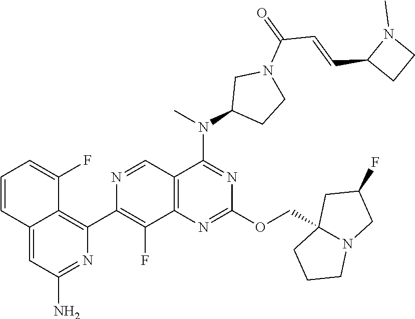 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ¹H NMR (400 MHz, CD₃CN) δ 9.28 (d, J = 2.8 Hz, 1H), 7.60-7.50 (m, 2H), 6.98 (d, J = 2.4 Hz, 1H), 6.94-6.80 (m, 2H), 6.61-6.44 (m, 1H), 5.51-5.22 (m, 4H), 4.37-4.25 (m, 2H), 4.24-4.06 (m, 1H), 4.05-3.85 (m, 2H), 3.85-3.71 (m, 2H), 3.70-3.53 (m, 2H), 3.52- 3.34 (m, 5H), 3.29 (d, J = 11.1 Hz, 2H), 3.25- 3.20 (m, 1H), 3.07-2.97 (m, 2H), 2.28- 2.05 (m, 7H), 1.99-1.82 (m, 3H) | 688.4 1.774 min |
| 136 | 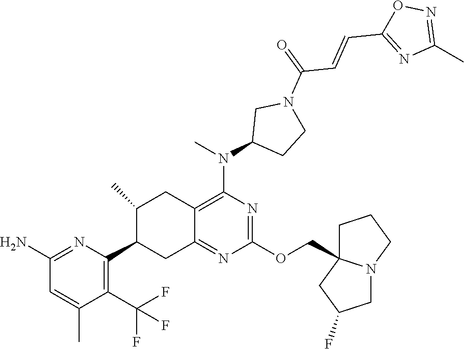 [SMILES: Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1] | ¹H NMR (400 MHz, CD₃CN) δ = 7.50-7.43 (m, 1H), 7.40-7.33 (m, 1H), 6.28 (s, 1H), 5.35-5.12 (m, 3H), 4.77-4.59 (m, 1H), 4.20- 3.89 (m, 4H), 3.87-3.62 (m, 2H), 3.51- 3.38 (m, 1H), 3.21-2.98 (m, 5H), 2.93 (d, J = 3.1 Hz, 3H), 2.90-2.81 (m, 2H), 2.70-2.61 (m, 1H), 2.55-2.45 (m, 1H), 2.41 (d, J = 5.3 Hz, 2H), 2.38 (q, J = 3.5 Hz, 3H), 2.29-2.19 (m, 1H), 2.14 (d, J = 4.1 Hz, 1H), 2.12-1.99 (m, 4H), 1.92-1.74 (m, 3H), 0.77 (d, J = 6.5 Hz, 3H) | 714.4 2.977 min |
| 137 | 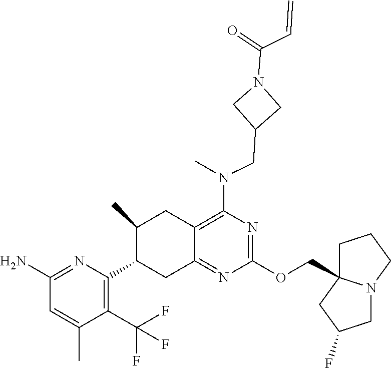 [SMILES: C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1] | ¹H NMR (400 MHz, CD₃CN) δ 6.35-6.22 (m, 2H), 6.20-6.12 (m, 1H), 5.67-5.59 (m, 1H), 5.35-5.15 (m, 3H), 4.28 (q, J = 8.5 Hz, 1H), 4.08-3.97 (m, 3H), 3.96-3.84 (m, 2H), 3.80-3.58 (m, 1H), 3.45 (ddd, J = 7.0, 13.9, 17.2 Hz, 1H), 3.22-3.10 (m, 3H), 3.08-2.94 (m, 6H), 2.93-2.78 (m, 2H), 2.66 (dd, J = 4.4, 15.8 Hz, 1H), 2.53-2.44 (m, 1H), 2.38 (q, J = 3.3 Hz, 3H), 2.15-2.06 (m, 3H), 2.04- 2.00 (m, 1H), 1.93-1.77 (m, 3H), 0.82- 0.67 (m, 3H) | 632.3 2.855 min |
| 138 | 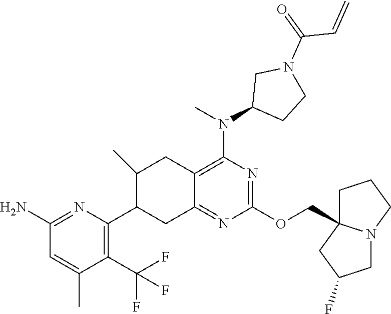 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1] | ¹H NMR (400 MHz, CD₃CN) δ 6.66-6.47 (m, 1H), 6.31-6.16 (m, 2H), 5.73-5.58 (m, 1H), 5.34-5.16 (m, 3H), 4.75-4.53 (m, 1H), 4.08-4.01 (m, 1H), 3.97-3.90 (m, 1H), 3.84- 3.72 (m, 1H), 3.66-3.47 (m, 1H), 3.45- 3.32 (m, 1H), 3.23-3.10 (m, 3H), 3.08-2.97 (m, 2H), 2.95-2.82 (m, 5H), 2.70-2.60 (m, 1H), 2.56-2.43 (m, 1H), 2.42-2.36 (m, 3H), 2.33-2.26 (m, 2H), 2.17-2.11 (m, 2H), 2.10- 2.01 (m, 3H), 1.94-1.80 (m, 3H), 0.77 (d, J = 6.5 Hz, 3H) | 632.4 1.012 min |
| 139 | 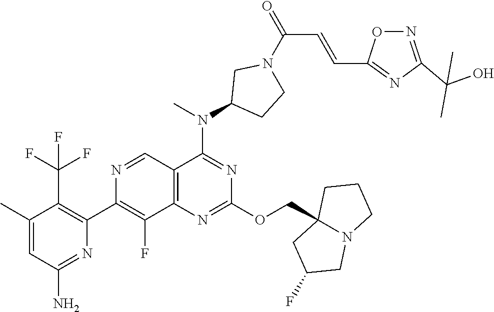 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F] | ¹H NMR (400 MHz, CD₃CN) δ 9.16 (d, J = 1.6 Hz, 1H), 7.55-7.36 (m, 2H), 6.59 (s, 1H), 5.48 (s, 2H), 5.35-5.19 (m, 1H), 4.28-4.12 (m, 3H), 4.02 (dd, J = 8.0, 12.9 Hz, 1H), 3.95- 3.84 (m, 1H), 3.82-3.72 (m, 1H), 3.66- 3.48 (m, 2H), 3.42 (s, 3H), 3.23-3.13 (m, 2H), 3.09 (d, J = 1.6 Hz, 1H), 2.97-2.86 (m, 1H), 2.46 (d, J = 1.6 Hz, 3H), 2.42-2.29 (m, 2H), 2.16-2.11 (m, 2H), 2.09-2.05 (m, 1H), 1.93-1.84 (m, 3H), 1.60 (d, J = 8.5 Hz, 6H) | 759.3 2.536 min |
| 140 | 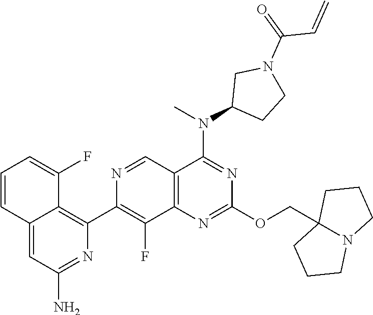 [SMILES: C=CC(=O)N1CC[C@@H](N(C)c2nc(OCC34CCCN3CCC4)nc3c(F)c(-c4nc(N)cc5cccc(F)c45)ncc23)C1] | ¹H NMR (400 MHz, CD₃CN) δ 9.21 (d, J = 3.6 Hz, 1H), 7.59-7.38 (m, 2H), 6.93 (d, J = 2.0 Hz, 1H), 6.89-6.79 (m, 1H), 6.61 (dt, J = 10.3, 16.2 Hz, 1H), 6.34-6.17 (m, 1H), 5.70 (ddd, J = 2.2, 7.8, 10.0 Hz, 1H), 5.47-5.31 (m, 1H), 5.20 (s, 2H), 4.18 (d, J = 4.1 Hz, 2H), 4.04-3.79 (m, 1H), 3.73-3.63 (m, 1H), 3.61-3.48 (m, 1H), 3.43 (s, 3H), 3.05-2.96 (m, 3H), 2.67-2.59 (m, 2H), 2.45-2.36 (m, 1H), 2.35-2.25 (m, 2H), 1.91-1.77 (m, 5H), 1.65 (td, J = 7.4, 12.1 Hz, 2H) | 601.3 2.170 min |
| 142 | 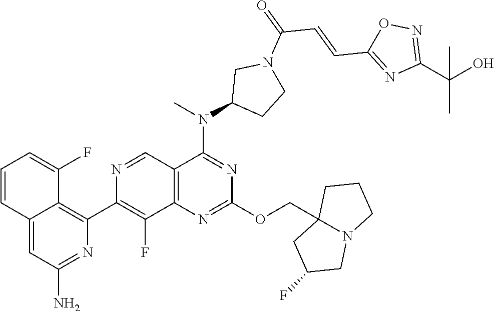 [SMILES: CN(c1nc(OCC23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1] | ¹H NMR (400 MHz, CD₃CN) δ 9.23 (s, 1H), 7.54-7.36 (m, 4H), 6.93 (d, J = 2.5 Hz, 1H), 6.88-6.81 (m, 1H), 5.48-5.16 (m, 4H), 4.30- 4.19 (m, 2H), 4.04 (dd, J = 8.2, 12.4 Hz, 1H), 3.91-3.78 (m, 1H), 3.69-3.59 (m, 1H), 3.54 (td, J = 8.6, 12.6 Hz, 1H), 3.45 (s, 3H), 3.27-3.09 (m, 3H), 2.99-2.88 (m, 1H), 2.44 (q, J = 8.6 Hz, 1H), 2.39-2.32 (m, 1H), 2.26- 2.21 (m, 2H), 2.14-2.06 (m, 2H), 1.90 (dd, J = 5.5, 11.6 Hz, 3H), 1.60 (d, J = 8.4 Hz, 6H) | 745.4 2.647 min |
| 143 | 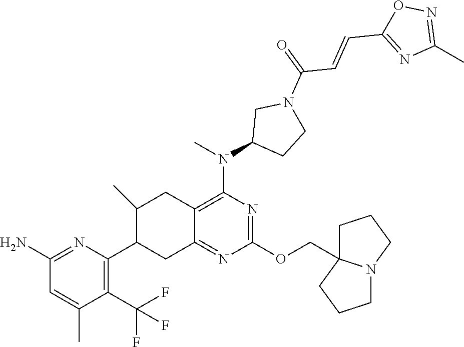 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1] | ¹H NMR (400 MHz, CD₃CN) δ 7.50-7.43 (m, 1H), 7.40-7.30 (m, 1H), 6.27 (s, 1H), 5.32 (s, 2H), 4.80-4.55 (m, 1H), 4.23-4.09 (m, 1H), 4.07-3.91 (m, 3H), 3.89-3.76 (m, 1H), 3.76-3.58 (m, 1H), 3.52-3.39 (m, 1H), 3.22-3.13 (m, 1H), 3.08-2.96 (m, 2H), 2.94- 2.90 (m, 3H), 2.89-2.80 (m, 1H), 2.69- 2.56 (m, 4H), 2.46 (dd, J = 4.9, 16.4 Hz, 3H), 2.41 (s, 1H), 2.37-2.33 (m, 2H), 2.20-2.03 (m, 3H), 1.95-1.87 (m, 2H), 1.86-1.71 (m, 4H), 1.67-1.52 (m, 2H), 0.75 (d, J = 6.4 Hz, 3H). | 696.4 2.748 min |
| 144 | 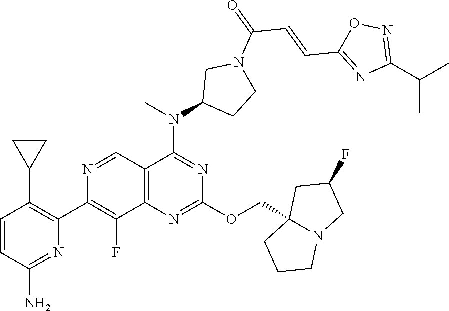 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1] | ¹H NMR (400 MHz, CD₃CN) δ 9.19 (d, J = 1.6 Hz, 1H), 7.49-7.33 (m, 2H), 7.18 (d, J = 8.5 Hz, 1H), 6.56 (d, J = 8.5 Hz, 1H), 5.43- 5.16 (m, 2H), 4.85 (s, 2H), 4.25-4.10 (m, 3H), 4.04-3.96 (m, 1H), 3.91-3.70 (m, 2H), 3.63-3.47 (m, 1H), 3.41 (s, 3H), 3.15-3.06 (m, 3H), 2.88 (quin, J = 7.6 Hz, 1H), 2.42- 2.28 (m, 2H), 2.12-1.97 (m, 3H), 1.91-1.80 (m, 3H), 1.71-1.63 (m, 1H), 1.32 (dd, J = 7.0, 8.6 Hz, 6H), 0.70-0.60 (m, 2H), 0.52- 0.42 (m, 2H). | 701.4 2.707 min |
| 151 | 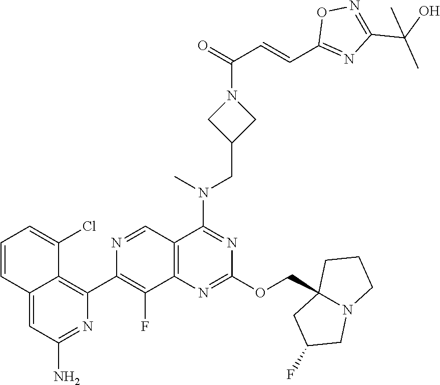 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12] | ¹H NMR (400 MHz, CD₃CN) δ 9.26-9.20 (m, 1H), 7.63 (d, J = 8.4 Hz, 1H), 7.41 (t, J = 7.8 Hz, 1H), 7.35-7.29 (m, 1H), 7.23 (d, J = 7.4 Hz, 1H), 7.19-7.14 (m, 1H), 6.92 (s, 1H), 5.33-5.13 (m, 3H), 4.47 (t, J = 8.6 Hz, 1H), 4.31-4.06 (m, 7H), 3.93 (dd, J = 5.6, 10.3 Hz, 1H), 3.57 (s, 3H), 3.27-3.20 (m, 1H), 3.17-3.10 (m, 2H), 3.08-3.04 (m, 1H), 2.89 (s, 1H), 2.06 (d, J = 17.8 Hz, 3H), 1.86 (s, 3H), 1.57 (s, 6H). | 761.3 2.666 min |
| 152 | 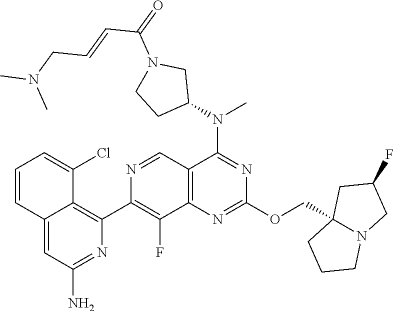 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | ¹H NMR (400 MHz, CD₃CN) δ 9.17 (d, J = 3.4 Hz, 1H), 7.63 (d, J = 8.4 Hz, 1H), 7.41 (t, J = 7.9 Hz, 1H), 7.23 (d, J = 7.3 Hz, 1H), 6.92 (s, 1H), 6.78-6.69 (m, 1H), 6.44-6.31 (m, 1H), 5.41-5.27 (m, 2H), 5.20-5.13 (m, 2H), 4.25-4.10 (m, 3H), 4.05 (s, 1H), 3.97- 3.76 (m, 3H), 3.68-3.49 (m, 3H), 3.40 (s, 3H), 3.15-3.03 (m, 6H), 2.93-2.83 (m, 2H), 2.10 (s, 5H), 1.92-1.82 (m, 3H) | 692.3/693.3 2.460 min |
| 154 | 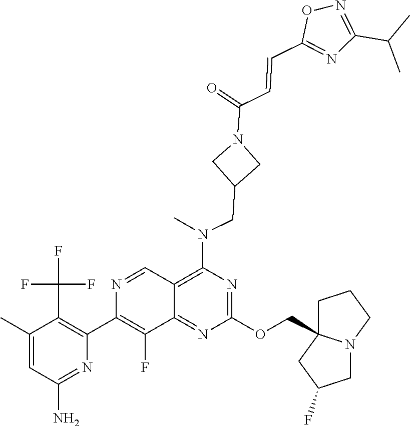 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C(F)(F)F] | ¹H NMR (400 MHz, DMSO-d₆) δ 9.22 (s, 1H), 7.32-7.24 (m, 1H), 7.22-7.15 (m, 1H), 6.82-6.76 (m, 2H), 6.51-6.48 (m, 1H), 5.34- 5.17 (m, 1H), 4.50 (t, J = 8.6 Hz, 1H), 4.31 (dd, J = 5.6, 8.8 Hz, 1H), 4.15-4.10 (m, 2H), 4.09-3.98 (m, 2H), 3.90 (dd, J = 5.4, 10.4 Hz, 1H), 3.57 (s, 3H), 3.22-3.18 (m, 1H), 3.17-3.11 (m, 1H), 3.10-3.06 (m, 2H), 3.01 (d, J = 5.0 Hz, 1H), 2.84-2.78 (m, 1H), 2.38- 2.35 (m, 3H), 2.17-2.02 (m, 2H), 2.00- 1.95 (m, 1H), 1.86-1.69 (m, 4H), 1.30-1.27 (m, 6H). | 743.4 2.972 min |
| 157 | 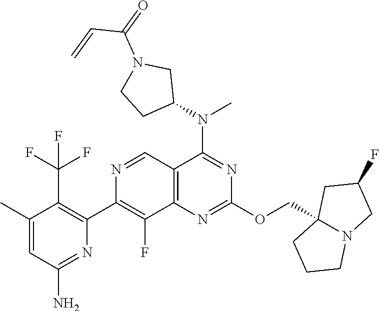 [SMILES: C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1] | ¹H NMR (400 MHz, CD₃CN) δ 9.15 (d, J = 2.1 Hz, 1H), 6.69-6.49 (m, 2H), 6.32-6.19 (m, 1H), 5.75-5.65 (m, 1H), 5.51-5.40 (m, 2H), 5.40-5.19 (m, 2H), 4.24-4.20 (m, 1H), 4.17-4.11 (m, 1H), 4.10-3.79 (m, 2H), 3.70- 3.63 (m, 1H), 3.60-3.44 (m, 1H), 3.40 (d, J = 1.0 Hz, 3H), 3.21-3.11 (m, 2H), 3.08 (s, 1H), 2.95-2.87 (m, 1H), 2.46 (d, J = 1.5 Hz, 3H), 2.42-2.33 (m, 1H), 2.33-2.19 (m, 2H), 2.14-2.11 (m, 1H), 2.10-2.04 (m, 1H), 1.94- 1.81 (m, 3H) | 633.3 2.527 min |
| 159 | 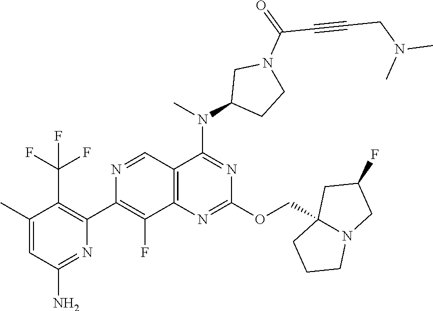 | ¹H NMR (400 MHz, CD₃CN) δ 9.15 (d, J = 4.6 Hz, 1H), 8.13 (s, 1H), 6.59 (s, 1H), 5.50- 5.43 (m, 2H), 5.40-5.18 (m, 2H), 4.26-4.20 (m, 1H), 4.18-4.13 (m, 1H), 4.05-3.86 (m, 1H), 3.78-3.70 (m, 1H), 3.53-3.46 (m, 1H), 3.46-3.38 (m, 5H), 3.23-3.13 (m, 2H), 3.10 (s, 1H), 2.98-2.88 (m, 1H), 2.46 (d, J = 1.3 Hz, 3H), 2.31 (s, 6H), 2.13-2.08 (m, 3H), 1.93-1.85 (m, 4H), 1.80 (td, J = 2.5, 4.9 Hz, 1H) | 688.3 1.811 min |
| 160 | 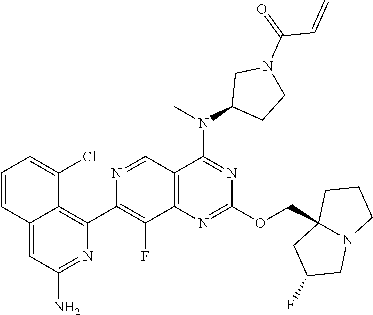 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1] | ¹H NMR (400 MHz, CD₃CN) δ 9.18-9.15 (m, 1H), 7.63 (d, J = 8.4 Hz, 1H), 7.41 (dd, J = 7.5, 8.3 Hz, 1H), 7.23 (dd, J = 0.9, 7.3 Hz, 1H), 6.92 (s, 1H), 6.65-6.51 (m, 1H), 6.27-6.19 (m, 1H), 5.71-5.63 (m, 1H), 5.45- 5.27 (m, 2H), 5.19-5.14 (m, 2H), 4.24- 4.03 (m, 3H), 3.97-3.77 (m, 2H), 3.70-3.62 (m, 1H), 3.58-3.50 (m, 1H), 3.43-3.39 (m, 3H), 3.18-3.10 (m, 2H), 2.93-2.87 (m, 1H), 2.42-2.26 (m, 3H), 2.05 (s, 2H), 1.91-1.77 (m, 3H) | 635.3/636.3 1.922 min |
| 161 | 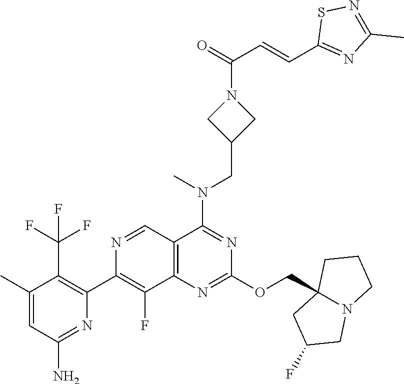 | ¹H NMR (400 MHz, CD₃CN) δ 9.22-9.16 (m, 1H), 7.72-7.62 (m, 1H), 7.11-6.99 (m, 1H), 6.60 (s, 1H), 5.34-5.16 (m, 1H), 4.48 (t, J = 8.3 Hz, 1H), 4.30-4.24 (m, 2H), 4.21 (dd, J = 3.8, 10.3 Hz, 2H), 4.11 (s, 2H), 3.96 (dd, J = 5.3, 10.4 Hz, 1H), 3.58 (s, 3H), 3.23 (d, J = 7.6 Hz, 2H), 3.19-3.14 (m, 2H), 2.91-2.85 (m, 1H), 2.66-2.59 (m, 3H), 2.43 (s, 3H), 2.21-2.03 (m, 3H), 1.91-1.78 (m, 3H) | 731.3 2.760 min |
| 163 | 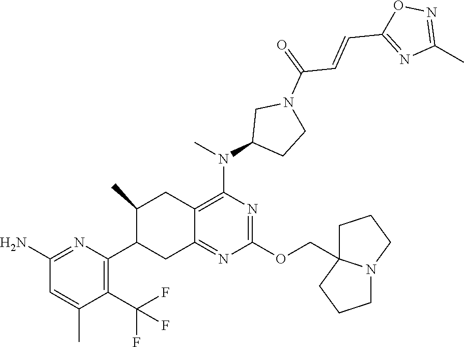 [SMILES: Cc1noc(/C=C/C(=O)N2CC[C@@H](N(C)c3nc(OCC45CCCN4CCC5)nc4c3C[C@H](C)C(c3nc(N)cc(C)c3C(F)(F)F)C4)C2)n1] | ¹H NMR (400 MHz, CD₃CN) δ 7.51-7.43 (m, 1H), 7.41-7.33 (m, 1H), 6.28 (s, 1H), 5.24 (s, 2H), 4.79-4.58 (m, 1H), 4.16 (dd, J = 8.0, 10.3 Hz, 1H), 4.03-3.89 (m, 3H), 3.87- 3.74 (m, 1H), 3.68 (d, J = 8.6 Hz, 1H), 3.52- 3.36 (m, 1H), 3.17 (td, J = 5.3, 10.3 Hz, 1H), 3.07-2.94 (m, 3H), 2.93 (d, J = 3.1 Hz, 3H), 2.89-2.81 (m, 1H), 2.66 (s, 4H), 2.41 (s, 1H), 2.40 (s, 1H), 2.38 (d, J = 3.5 Hz, 2H), 2.30-2.23 (m, 1H), 2.21-2.14 (m, 1H), 2.13- 2.03 (m, 2H), 1.91 (td, J = 6.1, 12.1 Hz, 2H), 1.86-1.71 (m, 4H), 1.66-1.54 (m, 2H), 0.77 (d, J = 6.3 Hz, 3H) | 696.4 2.742 min |
| 164 | 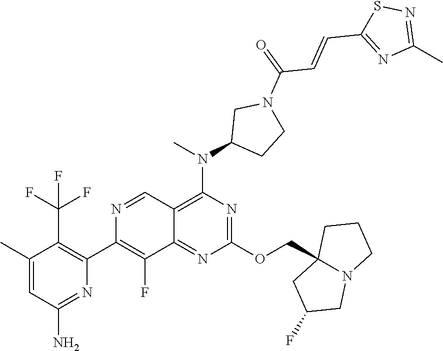 | ¹H NMR (400 MHz, CD₃CN) δ 9.14 (s, 1H), 7.80-7.68 (m, 1H), 7.39-7.24 (m, 1H), 6.57 (s, 1H), 5.53 (d, J = 6.4 Hz, 2H), 5.39-5.17 (m, 2H), 4.26-4.21 (m, 1H), 4.19-4.13 (m, 1H), 4.04-3.97 (m, 1H), 3.88-3.74 (m, 1H), 3.65-3.46 (m, 1H), 3.41 (s, 3H), 3.17-3.13 (m, 1H), 3.08 (d, J = 6.3 Hz, 1H), 2.95-2.85 (m, 1H), 2.66 (s, 1H), 2.63 (s, 1H), 2.45 (d, J = 1.6 Hz, 3H), 2.34-2.29 (m, 1H), 2.26- 2.16 (m, 4H), 2.15-2.10 (m, 1H), 2.10-2.02 (m, 1H), 1.95-1.77 (m, 4H) | 731.3 2.810 min |
| 165 | 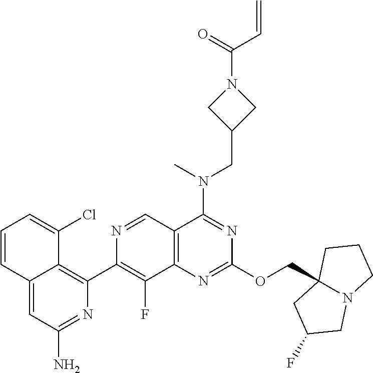 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1] | ¹H NMR (400 MHz, CD₃CN) δ 9.21 (s, 1H), 7.63 (d, J = 8.3 Hz, 1H), 7.41 (t, J = 7.9 Hz, 1H), 7.23 (d, J = 7.1 Hz, 1H), 6.92 (s, 1H), 6.33-6.23 (m, 1H), 6.20-6.12 (m, 1H), 5.62 (dd, J = 2.3, 10.3 Hz, 1H), 5.32 (s, 1H), 5.21- 4.96 (m, 3H), 4.34 (t, J = 8.6 Hz, 1H), 4.20- 4.08 (m, 5H), 3.84 (dd, J = 5.6, 10.0 Hz, 1H), 3.56 (s, 3H), 3.21-3.10 (m, 3H), 3.06 (s, 1H), 2.93-2.84 (m, 1H), 2.09-2.01 (m, 3H), 1.90-1.77 (m, 3H) | 635.3/636.3 1.906 min |
| 168 | 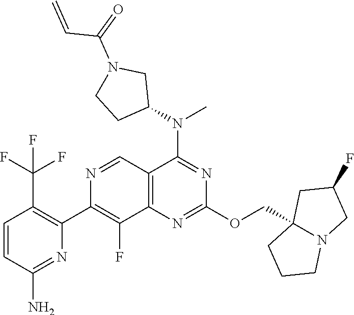 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1] | ¹H NMR (400 MHz, CD₃CN) δ 9.17 (d, J = 2.0 Hz, 1H), 7.84 (d, J = 8.9 Hz, 1H), 6.73 (d, J = 8.8 Hz, 1H), 6.60 (dt, J = 10.4, 16.8 Hz, 1H), 6.31-6.18 (m, 1H), 5.76-5.65 (m, 1H), 5.60 (s, 2H), 5.46-5.17 (m, 2H), 4.25-4.12 (m, 2H), 4.08 (dd, J = 8.1, 10.4 Hz, 1H), 3.99- 3.92 (m, 1H), 3.90-3.78 (m, 1H), 3.73- 3.61 (m, 1H), 3.60-3.44 (m, 1H), 3.41 (s, 3H), 3.23-3.04 (m, 3H), 2.98-2.86 (m, 1H), 2.45-2.27 (m, 2H), 2.25-2.19 (m, 1H), 2.13- 2.04 (m, 2H), 1.95-1.83 (m, 3H) | 619.3 2.421 min |
| 170 | 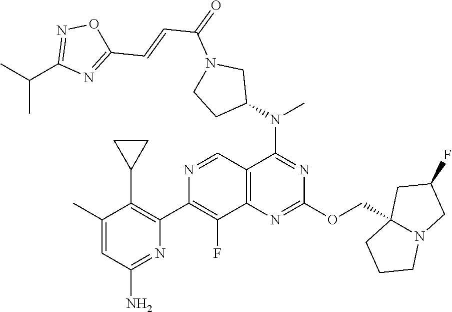 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1] | ¹H NMR (400 MHz, CD₃CN) δ 9.21 (d, J = 2.1 Hz, 1H), 7.53-7.37 (m, 2H), 6.52-6.47 (m, 1H), 5.50-5.18 (m, 2H), 4.88-4.76 (m, 2H), 4.30-4.19 (m, 2H), 4.18-4.13 (m, 1H), 4.08-3.87 (m, 2H), 3.83-3.75 (m, 1H), 3.67- 3.51 (m, 1H), 3.45 (s, 3H), 3.19-3.14 (m, 2H), 3.13-3.07 (m, 1H), 2.97-2.87 (m, 1H), 2.43 (s, 3H), 2.38-2.32 (m, 1H), 2.26-2.20 (m, 2H), 2.15-2.12 (m, 1H), 2.10-2.05 (m, 1H), 1.94-1.83 (m, 3H), 1.78-1.69 (m, 1H), 1.37 (d, J = 7.0 Hz, 3H), 1.35 (d, J = 7.0 Hz, 3H), 0.59-0.52 (m, 2H), 0.06-0.00 (m, 2H) | 715.4 2.918 min |
| 171 | 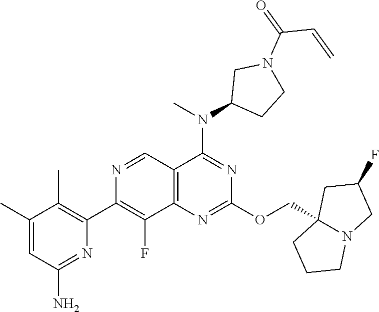 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1] | ¹H NMR (400 MHz, CD₃CN) δ 9.17 (s, 1H), 8.18 (s, 1H), 6.63-6.50 (m, 1H), 6.49 (s, 1H), 6.27-6.17 (m, 1H), 5.71-5.61 (m, 1H), 5.42-5.22 (m, 2H), 4.36-4.25 (m, 2H), 3.92 (dd, J = 8.1, 12.8 Hz, 1H), 3.88-3.75 (m, 1H), 3.62 (s, 1H), 3.57-3.42 (m, 1H), 3.38 (s, 3H), 3.36 (s, 1H), 3.34-3.16 (m, 2H), 2.99 (d, J = 5.6 Hz, 1H), 2.40-2.25 (m, 3H), 2.24 (s, 3H), 2.21 (s, 1H), 2.19-2.10 (m, 1H), 2.03-1.94 (m, 3H), 1.93-1.85 (m, 3H). | 579.3 2.497 min |
| 172 | 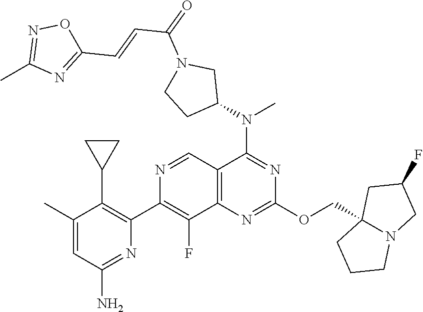 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1] | ¹H NMR (400 MHz, CD₃CN) δ 9.20-9.14 (m, 1H), 7.50-7.32 (m, 2H), 6.48-6.43 (m, 1H), 5.18 (d, J = 2.6 Hz, 2H), 4.79-4.71 (m, 2H), 4.22-4.18 (m, 1H), 4.16 (s, 1H), 4.04- 3.96 (m, 1H), 3.91-3.82 (m, 1H), 3.80-3.70 (m, 1H), 3.64-3.45 (m, 1H), 3.41 (s, 3H), 3.19-3.08 (m, 2H), 3.07-3.02 (m, 1H), 2.93- 2.82 (m, 1H), 2.44-2.40 (m, 1H), 2.39 (s, 4H), 2.37 (s, 2H), 2.35-2.27 (m, 1H), 2.22 (d, J = 4.4 Hz, 1H), 2.10 (d, J = 2.8 Hz, 1H), 2.07-1.99 (m, 1H), 1.91-1.78 (m, 3H), 1.74- 1.64 (m, 1H), 0.57-0.48 (m, 2H), −0.01 (d, J = 4.5 Hz, 2H) | 687.4 2.723 min |
| 173 | 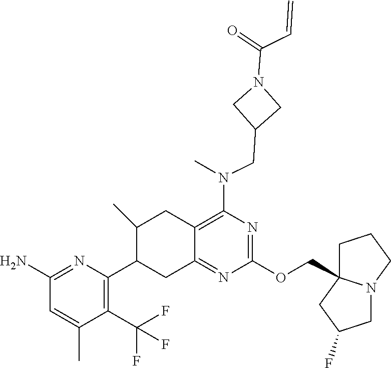 [SMILES: C=CC(=O)N1CC(CN(C)c2nc(OC[C@@]34CCCN3C[C@H](F)C4)nc3c2CC(C)C(c2nc(N)cc(C)c2C(F)(F)F)C3)C1] | ¹H NMR (400 MHz, CD₃CN) δ 6.36-6.22 (m, 2H), 6.21-6.09 (m, 1H), 5.67-5.57 (m, 1H), 5.35-5.12 (m, 3H), 4.28 (q, J = 8.6 Hz, 1H), 4.09-3.97 (m, 3H), 3.95-3.83 (m, 2H), 3.80-3.57 (m, 1H), 3.52-3.38 (m, 1H), 3.23- 3.09 (m, 3H), 3.08-2.95 (m, 6H), 2.94- 2.77 (m, 2H), 2.66 (dd, J = 4.3, 15.8 Hz, 1H), 2.54-2.43 (m, 1H), 2.38 (q, J = 3.3 Hz, 3H), 2.18-2.13 (m, 3H), 2.01 (s, 1H), 1.93-1.75 (m, 3H), 0.77 (d, J = 6.4 Hz, 3H) | 632.3 2.857 min |
| 175 | 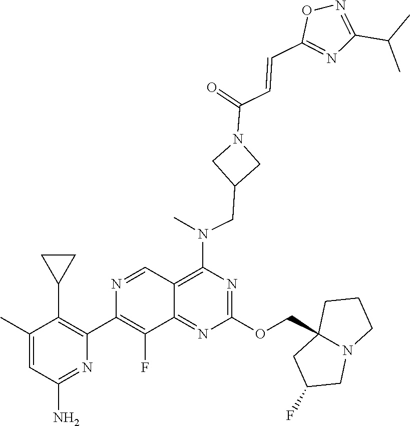 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C1CC1] | ¹H NMR (400 MHz, CD₃CN) δ 9.24 (s, 1H), 7.37-7.30 (m, 1H), 7.21-7.13 (m, 1H), 6.52 (s, 1H), 5.40-5.15 (m, 1H), 4.54-4.47 (m, 1H), 4.34-4.10 (m, 6H), 4.01-3.93 (m, 1H), 3.62-3.57 (m, 3H), 3.33-3.17 (m, 1H), 3.17- 3.06 (m, 4H), 2.93-2.86 (m, 1H), 2.49- 2.36 (m, 3H), 2.26-2.01 (m, 3H), 1.93-1.69 (m, 4H), 1.37-1.22 (m, 6H), 0.55 (d, J = 8.5 Hz, 2H), −0.01 (d, J = 4.8 Hz, 2H) | 715.4 1.948 min |
| 176 | 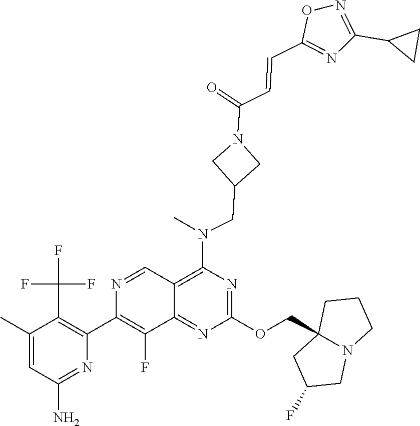 [SMILES: Cc1cc(N)nc(-c2ncc3c(N(C)CC4CN(C(=O)/C=C/c5nc(C6CC6)no5)C4)nc(OC[C@@]45CCCN4C[C@H](F)C5)nc3c2F)c1C(F)(F)F] | ¹H NMR (400 MHz, DMSO-d₆) δ 9.21 (s, 1H), 7.18 (q, J = 15.8 Hz, 2H), 6.81 (s, 2H), 6.51 (s, 1H), 5.35-5.15 (m, 1H), 4.48 (t, J = 8.6 Hz, 1H), 4.30 (dd, J = 5.8, 8.8 Hz, 1H), 4.22-3.98 (m, 5H), 3.90 (dd, J = 5.7, 10.4 Hz, 1H), 3.56 (s, 3H), 3.23-3.12 (m, 1H), 3.10-2.97 (m, 3H), 2.86-2.76 (m, 1H), 2.37 (d, J = 1.1 Hz, 3H), 2.21-2.07 (m, 2H), 2.06- 1.94 (m, 2H), 1.85-1.72 (m, 3H), 1.13- 1.06 (m, 2H), 0.96-0.88 (m, 2H) | 741.3 2.669 min |
| 177 | 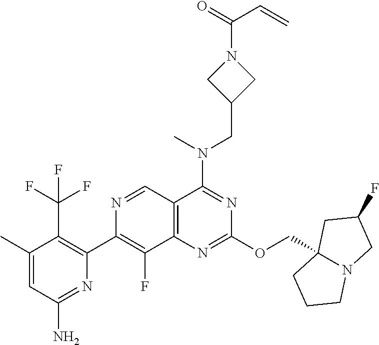 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1] | ¹H NMR (400 MHz, CD₃CN) δ 9.19 (s, 1H), 6.59 (s, 1H), 6.35-6.24 (m, 1H), 6.22-6.12 (m, 1H), 5.65 (dd, J = 1.7, 10.2 Hz, 1H), 5.47 (s, 2H), 5.38-5.18 (m, 1H), 4.35 (t, J = 8.4 Hz, 1H), 4.20 (s, 1H), 4.15-4.00 (m, 4H), 3.86 (dd, J = 5.6, 9.8 Hz, 1H), 3.56 (s, 3H), 3.20-3.13 (m, 3H), 3.08 (s, 1H), 2.95-2.87 (m, 1H), 2.46 (s, 3H), 2.31-2.20 (m, 1H), 2.11 (d, J = 1.6 Hz, 1H), 2.09-2.04 (m, 1H), 1.94-1.78 (m, 4H). | 633.3 2.476 min |

### BIOLOGICAL EXAMPLES

### Example 179: Inhibition of KRAS^{G12C} and cRAF Binding

The AlphaScreen technology was used to determine IC₅₀s for compound inhibition of KRAS G12C (present as the Cys-light (C51S, C80L and C118S), truncated version comprising amino acids 1-169) and cRAF interaction. Compounds were diluted in 100% DMSO and each compound concentration was spotted at 200 nl/well onto low volume, white 384 well plates. The KRAS G12C contained a biotin-AviTag and the cRaf, as Ras-binding domain (amino acids 50-131, RBD), was GST-tagged. KRAS G12C was preloaded with the GTP analogue Guanosine 5′-[β,γ-imido]triphosphate (GMPPNP). The KRAS G12C was diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates were incubated for 2 hours. A mixture of RBD and the AlphaScreen streptavidin donor and glutathione acceptor beads diluted in 25 mM Hepes, pH 7.4, 150 mM NaCl, 5 mM MgCl₂, 0.01% TritonX-100 and 2% DMSO was then added at 10 ul/well and incubated for 60-90 minutes before the samples were read for emission at 570 nm after excitation of the donor beads at 680 nm. All incubations were performed at room temperature. The final top compound concentration was 50 μM with 1:3 titrations for 10-point dose response curves. Final assay conditions were 0.5 nM KRAS G12C, 0.75 nM RBD and 5 μg/ml each of AlphaScreen donor and acceptor beads. IC₅₀s were determined using nonlinear regression fit of [inhibitor] vs. response (4 parameters).

A counter assay was also set up to rule out inhibitors of the AlphaScreen technology itself. Compound plates were incubated for 2 hours as above with buffer only. The AlphaScreen beads were added as above except biotin-AviTag-GST was substituted for the RBD. Samples were read and analyzed as above.

Results for compounds are shown in Table 1.

TABLE 1
Inhibition of KRAS G12C and cRAF Binding (IC₅₀)

| SyntheticExample | Structure | IC₅₀ |
|---|---|---|
| 1 | 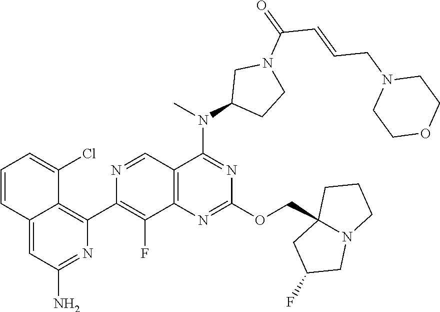 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/CN2CCOCC2)C1] | ++ |
| 2 (or) azetidine (abs) pyrrolidine (abs) fluoropyrolizine | 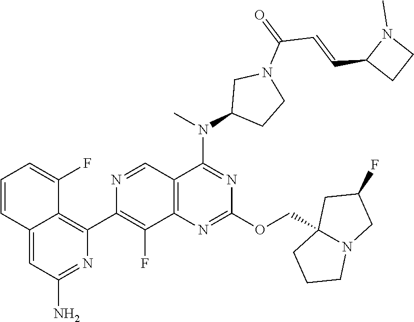 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ++ |
| 3 (or) azetidine (abs) pyrrolidine (abs) fluoropyrolizine | 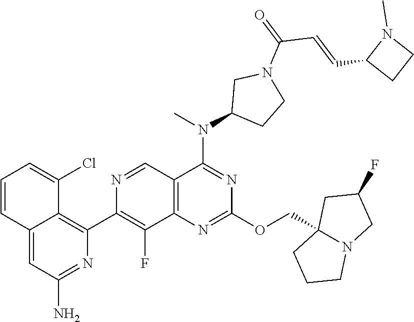 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 4 | 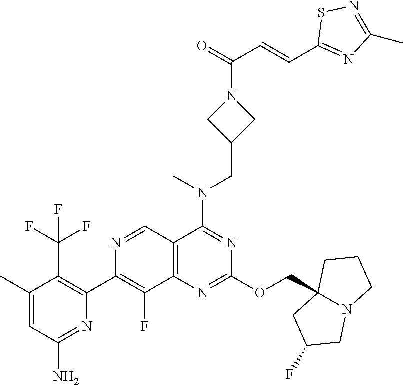 | +++ |
| 5 | 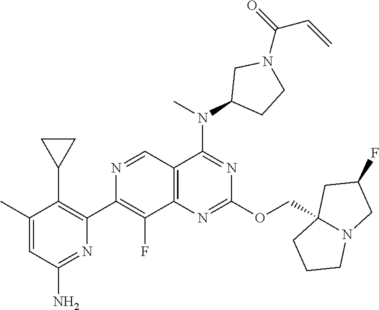 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1] | ++ |
| 6 (or) azetidine (abs) pyrrolidine (abs) fluoropyrolizine | 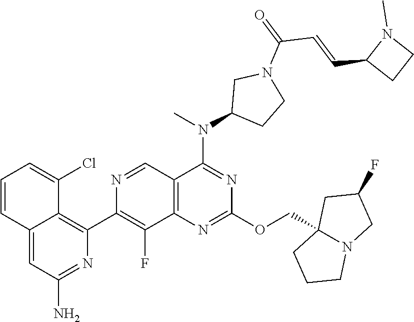 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | ++++ |
| 7 (or) azetidine (abs) pyrrolidine (abs) fluoropyrolizine | 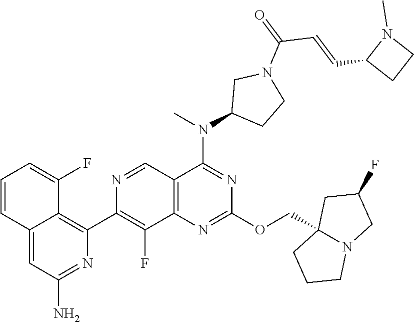 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ++++ |
| 8 | 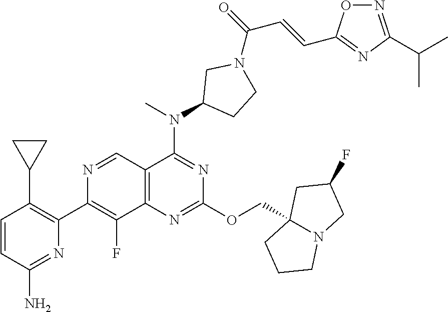 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1] | +++ |
| 9 (abs) pyrrolidine (or) both cyclohexenyl stereocenters | 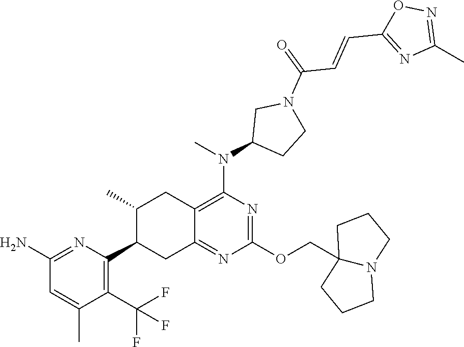 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1] | ++ |
| 10 | 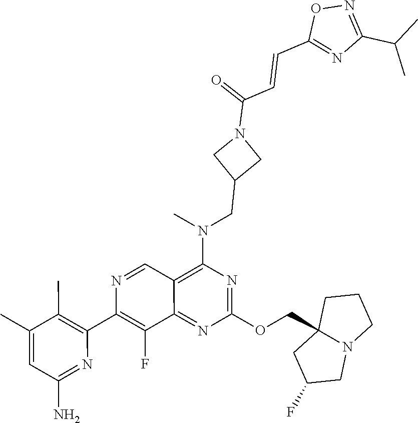 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C] | ++ |
| 11 | 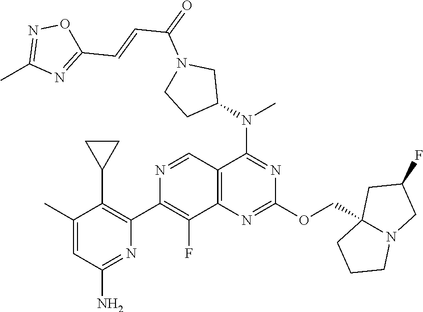 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1] | ++++ |
| 12 | 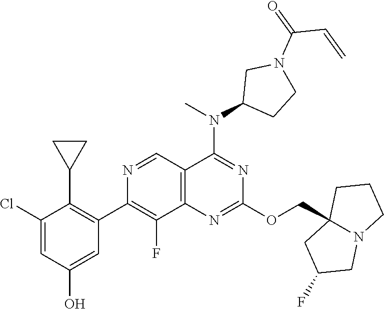 [SMILES: C=CC(=O)N1CC[C@@H](N(C)c2nc(OC[C@@]34CCCN3C[C@H](F)C4)nc3c(F)c(-c4cc(O)cc(Cl)c4C4CC4)ncc23)C1] | ++++ |
| 13 | 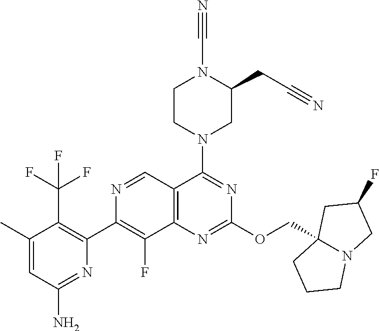 [SMILES: Cc1cc(N)nc(-c2ncc3c(N4CCN(C#N)[C@@H](CC#N)C4)nc(OC[C@@]45CCCN4C[C@H](F)C5)nc3c2F)c1C(F)(F)F] | +++ |
| 14 | 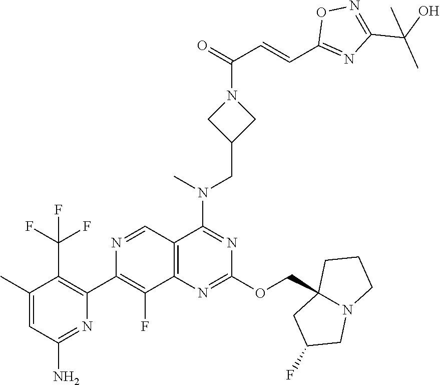 [SMILES: Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F] | +++ |
| 15 | 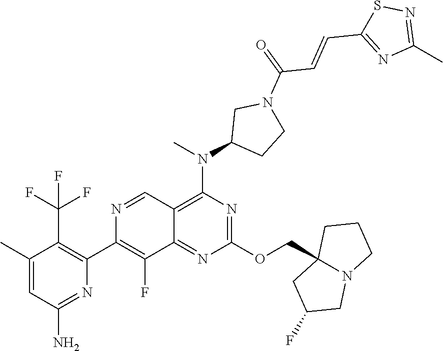 | +++ |
| 16 | 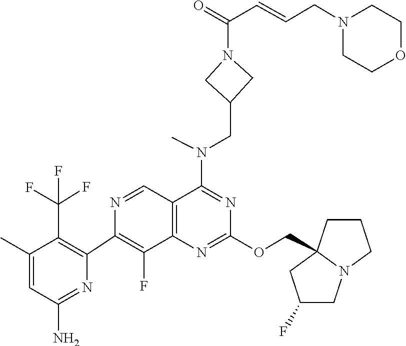 [SMILES: Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F] | ++ |
| 17 (or) both fused cyclohexyl stereocenters (abs) pyrrolidine | 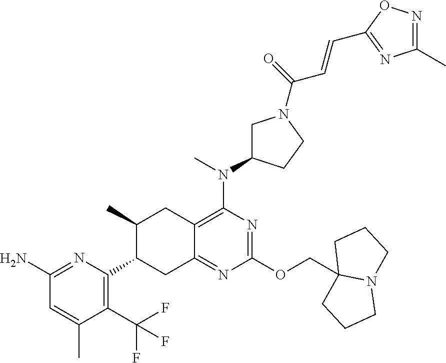 [SMILES: Cc1noc(/C=C/C(=O)N2CC[C@@H](N(C)c3nc(OCC45CCCN4CCC5)nc4c3C[C@H](C)[C@@H](c3nc(N)cc(C)c3C(F)(F)F)C4)C2)n1] | +++ |
| 18 (or) both fused cyclohexyl stereocenters (abs) pyrrolidine | 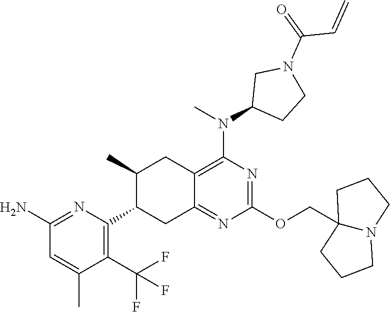 [SMILES: C=CC(=O)N1CC[C@@H](N(C)c2nc(OCC34CCCN3CCC4)nc3c2C[C@H](C)[C@@H](c2nc(N)cc(C)c2C(F)(F)F)C3)C1] | ++ |
| 19 | 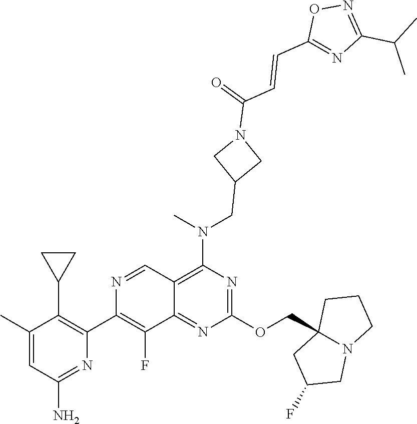 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C1CC1] | ++ |
| 20 | 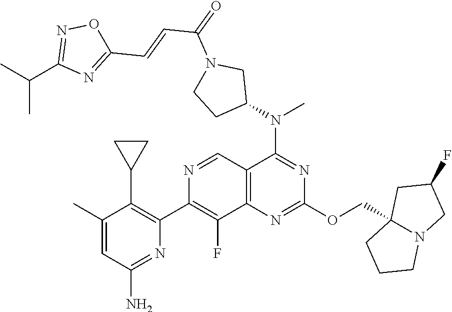 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1] | +++ |
| 21 | 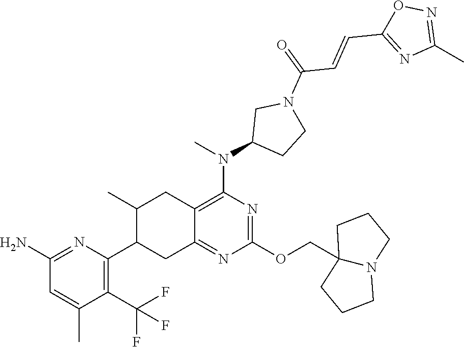 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1] | ++++ |
| 22 | 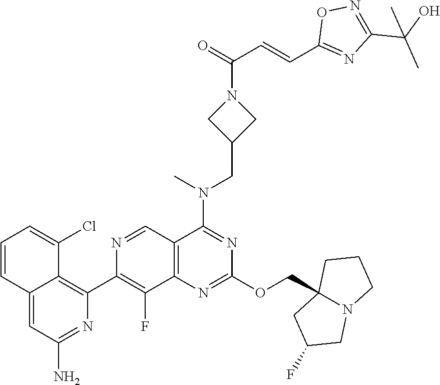 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12] | ++++ |
| 23 | 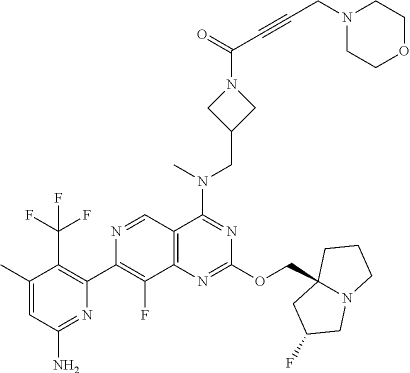 [SMILES: Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F] | +++ |
| 24 | 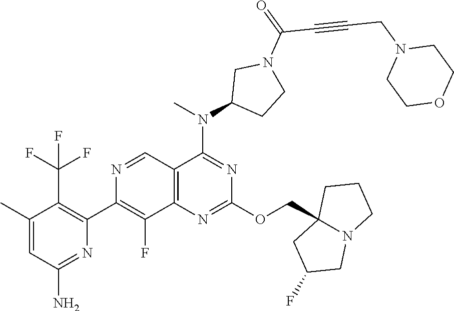 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F] | +++ |
| | (formate salt) | |
| 25 | 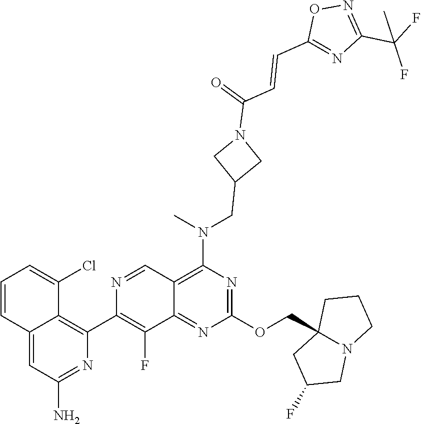 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12] | ++++ |
| 26 (or) azetidine (abs) pyrrolidine (abs) fluoropyrolizine | 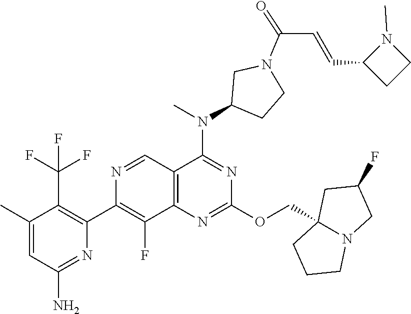 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F] | ++++ |
| 27 (or) azetidine (abs) pyrrolidine (abs) fluoropyrolizine | 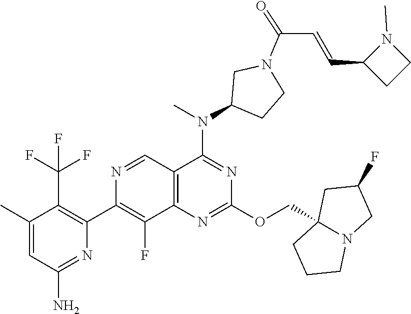 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F] | ++++ |
| 28 | 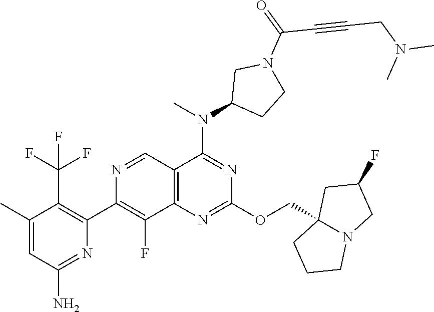 | ++++ |
| 29 | 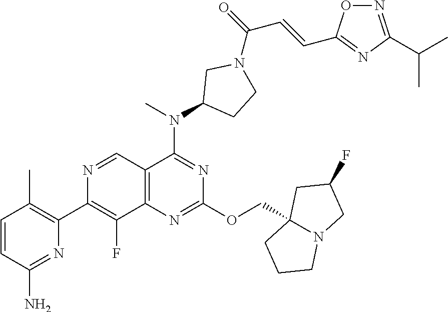 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F] | +++ |
| 30 | 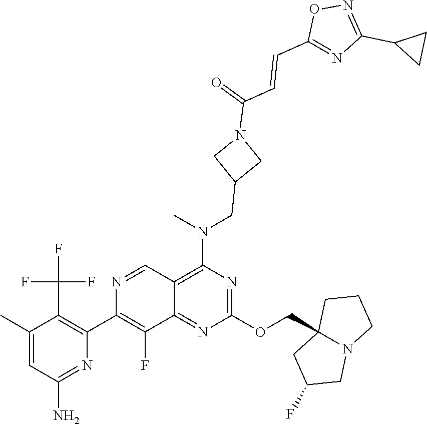 [SMILES: Cc1cc(N)nc(-c2ncc3c(N(C)CC4CN(C(=O)/C=C/c5nc(C6CC6)no5)C4)nc(OC[C@@]45CCCN4C[C@H](F)C5)nc3c2F)c1C(F)(F)F] | ++++ |
| 31 | 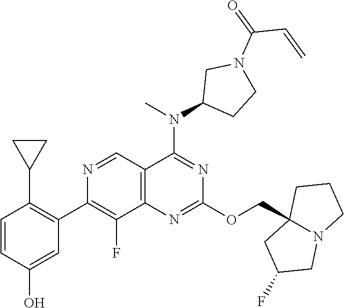 [SMILES: C=CC(=O)N1CC[C@@H](N(C)c2nc(OC[C@@]34CCCN3C[C@H](F)C4)nc3c(F)c(-c4cc(O)ccc4C4CC4)ncc23)C1] | +++ |
| | (formate salt) | |
| 32 | 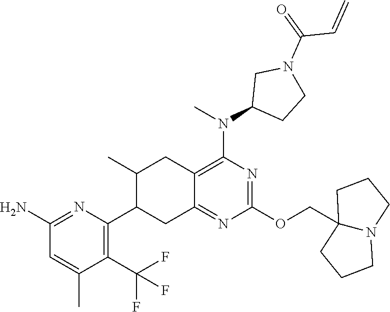 [SMILES: C=CC(=O)N1CC[C@@H](N(C)c2nc(OCC34CCCN3CCC4)nc3c2CC(C)C(c2nc(N)cc(C)c2C(F)(F)F)C3)C1] | +++ |
| 33 | 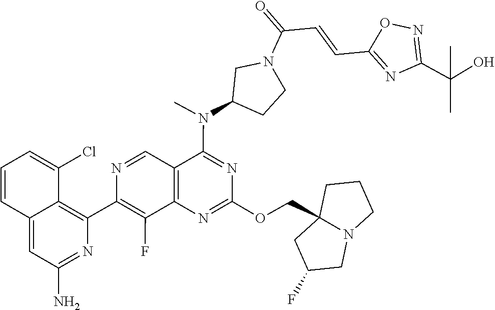 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1] | ++++ |
| 34 | 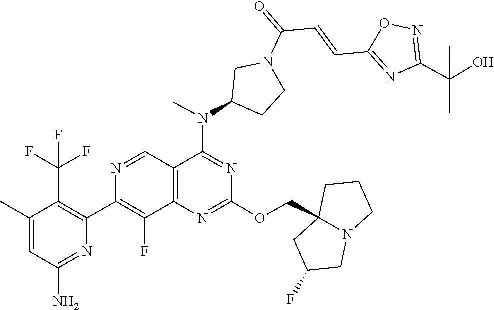 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F] | ++++ |
| 35 | 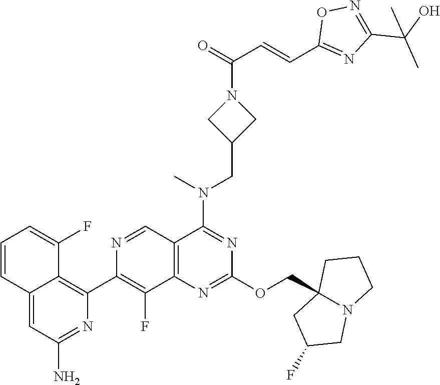 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12] | ++++ |
| 36 | 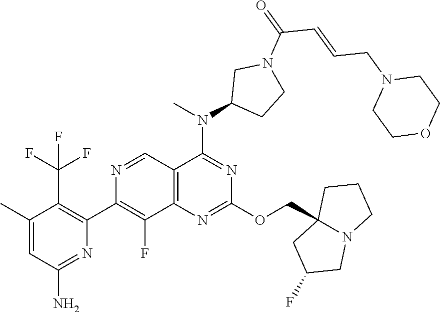 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F] | ++ |
| 37 | 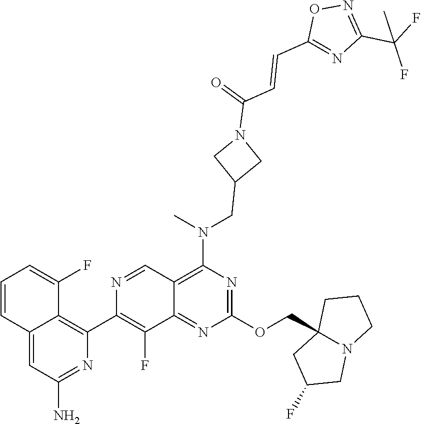 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12] | ++++ |
| 38 | 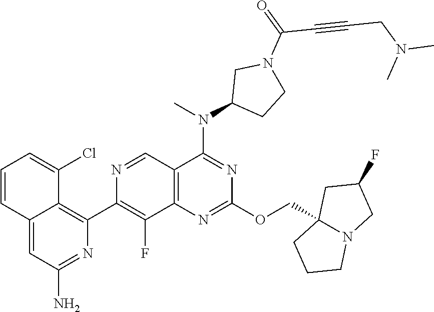 [SMILES: CN(C)CC#CC(=O)N1CC[C@@H](N(C)c2nc(OC[C@@]34CCCN3C[C@H](F)C4)nc3c(F)c(-c4nc(N)cc5cccc(Cl)c45)ncc23)C1] | ++++ |
| 39 | 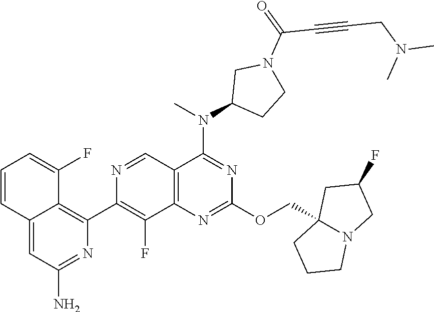 [SMILES: CN(C)CC#CC(=O)N1CC[C@@H](N(C)c2nc(OC[C@@]34CCCN3C[C@H](F)C4)nc3c(F)c(-c4nc(N)cc5cccc(F)c45)ncc23)C1] | ++++ |
| 40 (or) pyrrolidine methyl (abs) NMe pyrrolidine (abs) fluoropyrolizine | 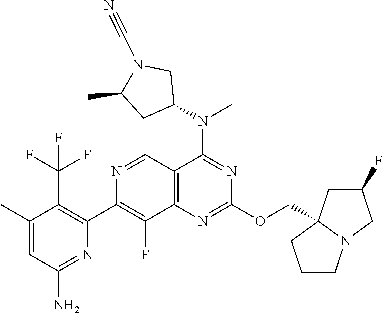 [SMILES: Cc1cc(N)nc(-c2ncc3c(N(C)[C@@H]4C[C@@H](C)N(C#N)C4)nc(OC[C@@]45CCCN4C[C@H](F)C5)nc3c2F)c1C(F)(F)F] | +++ |
| 41 | 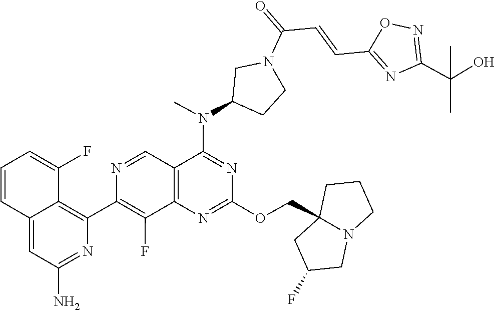 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1] | ++++ |
| 42 | 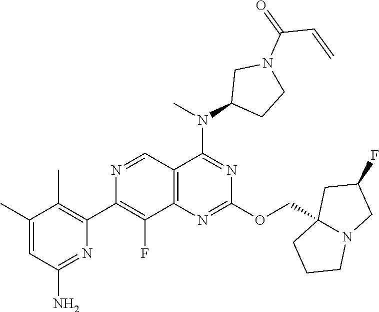 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1] | ++ |
| 43 | 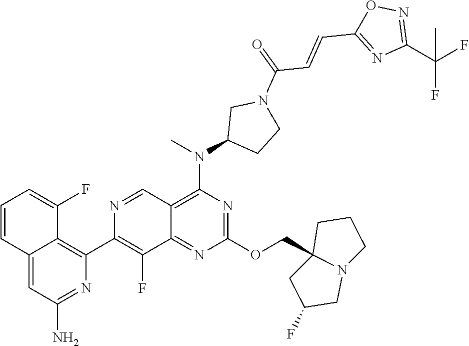 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1] | ++++ |
| | (formate salt) | |
| 44 | 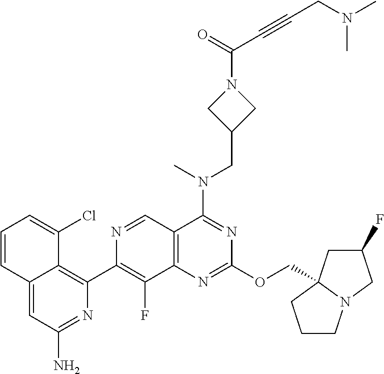 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1] | +++ |
| 45 | 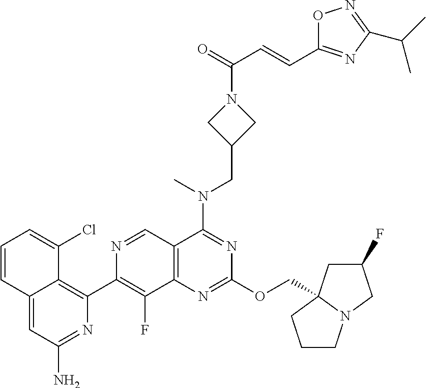 [SMILES: CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1] | ++ |
| 46 | 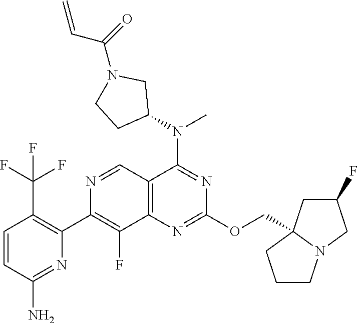 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1] | ++ |
| 47 | 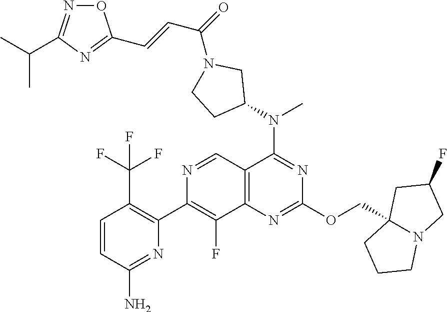 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1] | +++ |
| 48 | 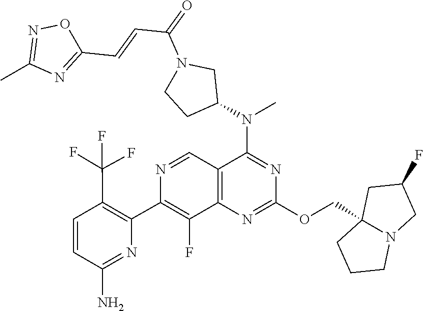 [SMILES: Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1] | +++ |
| 49 | 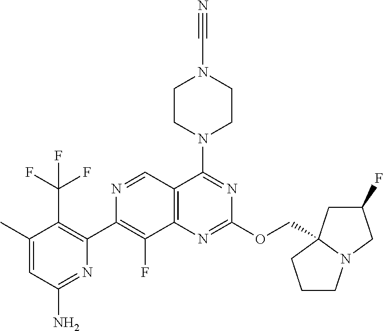 [SMILES: Cc1cc(N)nc(-c2ncc3c(N4CCN(C#N)CC4)nc(OC[C@@]45CCCN4C[C@H](F)C5)nc3c2F)c1C(F)(F)F] | ++ |
| 50 (or) both cyclohexenyl stereocenters | 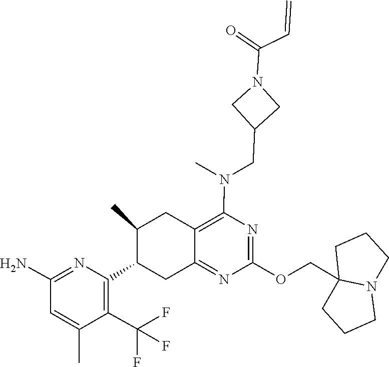 [SMILES: C=CC(=O)N1CC(CN(C)c2nc(OCC34CCCN3CCC4)nc3c2C[C@H](C)[C@@H](c2nc(N)cc(C)c2C(F)(F)F)C3)C1] | + |
| 51 | 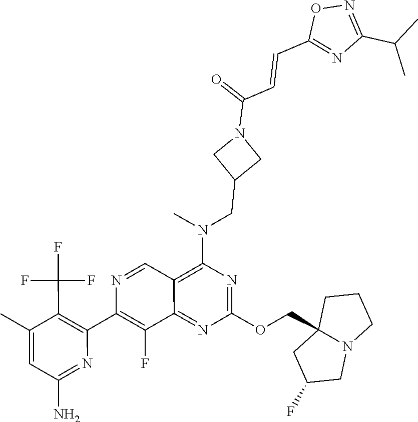 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C(F)(F)F] | ++++ |
| 52 | 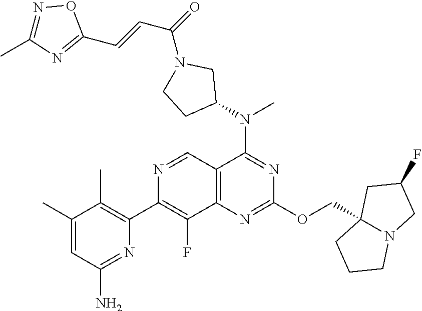 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1] | +++ |
| 53 | 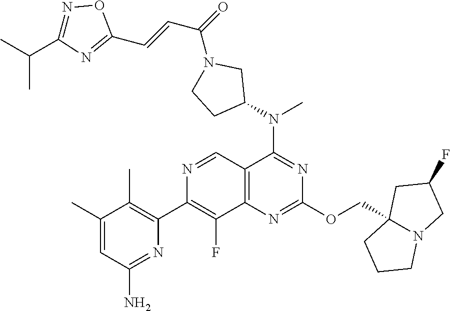 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C] | +++ |
| 54 | 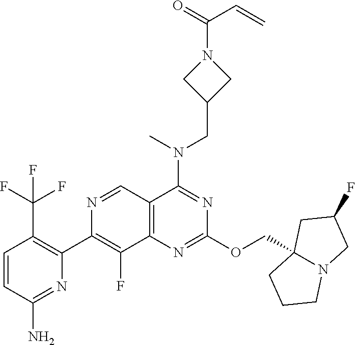 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1] | ++ |
| 55 | 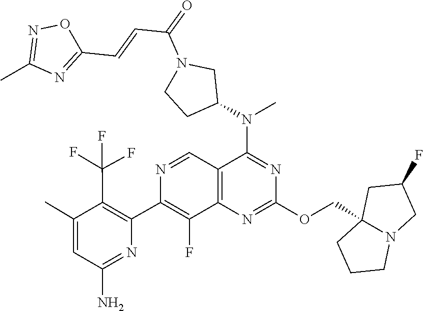 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1] | ++++ |
| 56 | 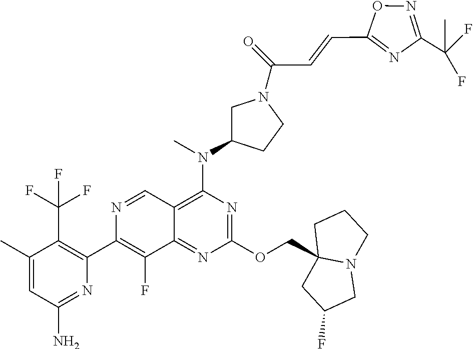 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F] | ++++ |
| 57 (or) both cyclohexenyl stereocenters (abs) pyrrolidine (abs) fluoropyrolizine | 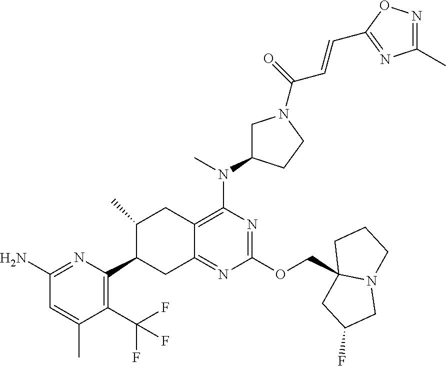 [SMILES: Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1] | ++++ |
| 58 (or) both cyclohexenyl stereocenters (abs) fluoropyrolizine | 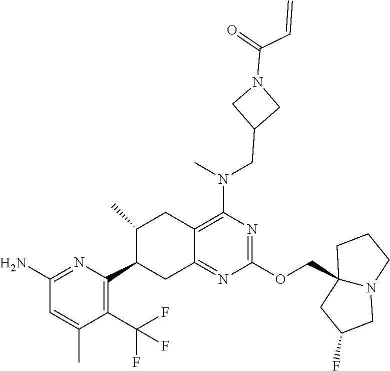 [SMILES: C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1] | ++ |
| 59 (or) both cyclohexenyl stereocenters (abs) pyrrolidine (abs) fluoropyrolizine | 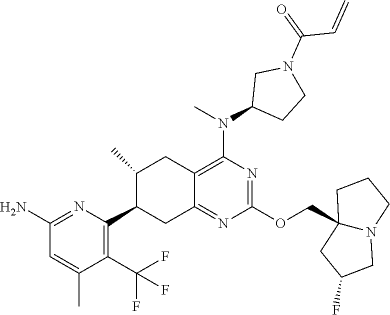 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1] | ++ |
| 60 | 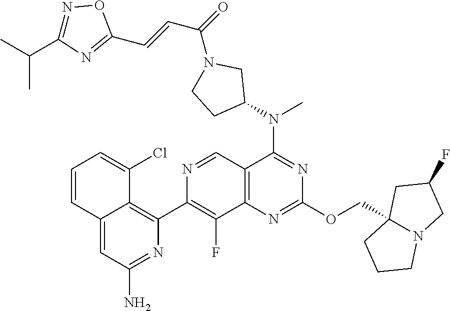 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1] | ++++ |
| 61 | 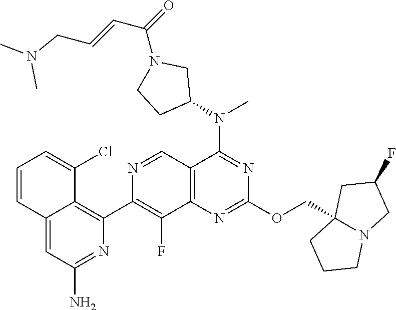 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 62 | 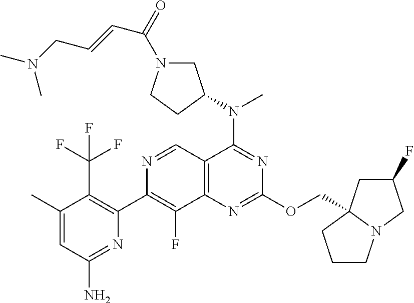 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F] | +++ |
| 63 | 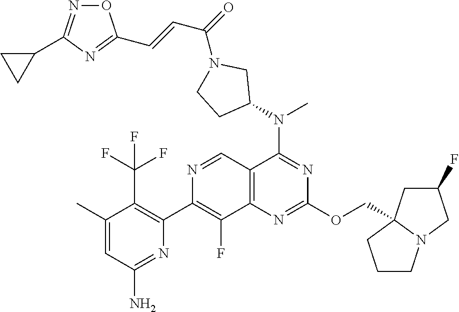 [SMILES: Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F] | ++++ |
| 64 | 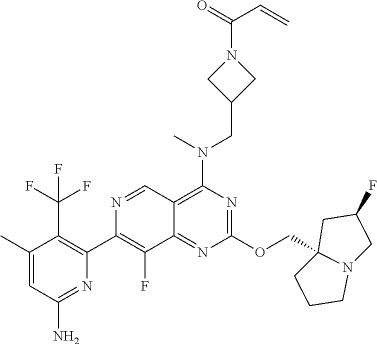 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1] | +++ |
| 65 | 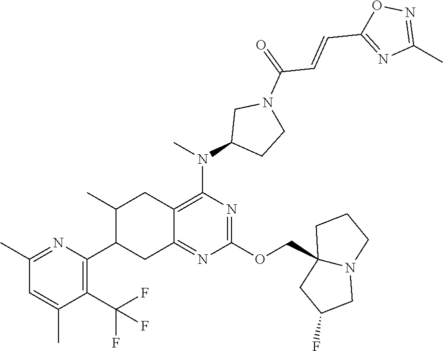 | ++++ |
| 66 | 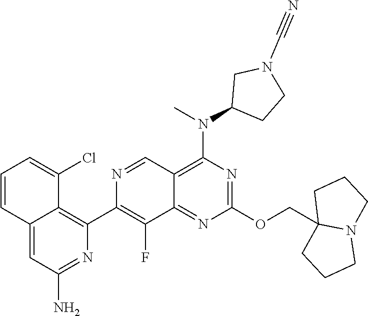 [SMILES: CN(c1nc(OCC23CCCN2CCC3)nc2c(F)c(-c3nc(N)cc4cccc(Cl)c34)ncc12)[C@@H]1CCN(C#N)C1] | +++ |
| 67 | 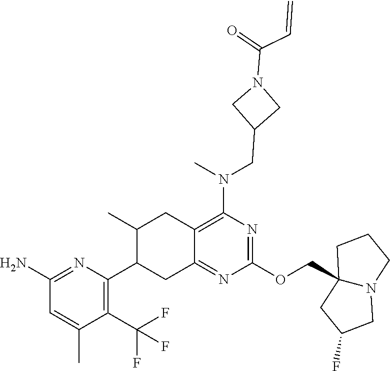 [SMILES: C=CC(=O)N1CC(CN(C)c2nc(OC[C@@]34CCCN3C[C@H](F)C4)nc3c2CC(C)C(c2nc(N)cc(C)c2C(F)(F)F)C3)C1] | + |
| 68 | 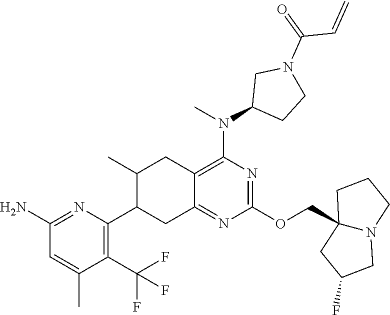 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1] | ++ |
| 69 | 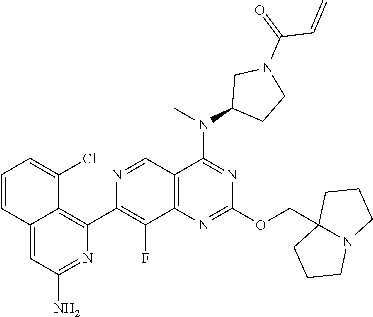 [SMILES: C=CC(=O)N1CC[C@@H](N(C)c2nc(OCC34CCCN3CCC4)nc3c(F)c(-c4nc(N)cc5cccc(Cl)c45)ncc23)C1] | ++ |
| 70 | 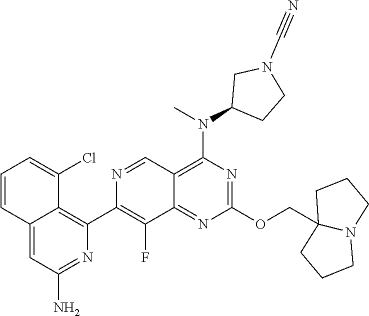 [SMILES: CN(c1nc(OCC23CCCN2CCC3)nc2c(F)c(-c3nc(N)cc4cccc(Cl)c34)ncc12)[C@@H]1CCN(C#N)C1] | ++ |
| 71 | 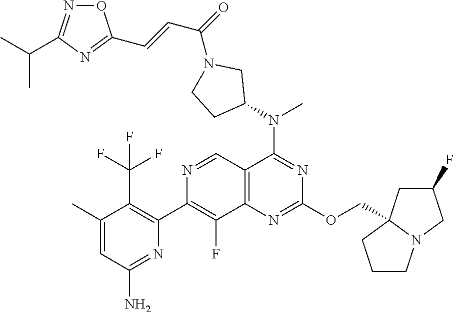 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F] | ++++ |
| 72 | 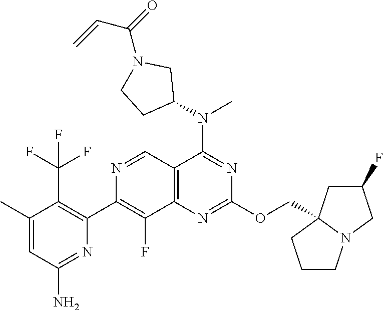 [SMILES: C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1] | +++ |
| 73 | 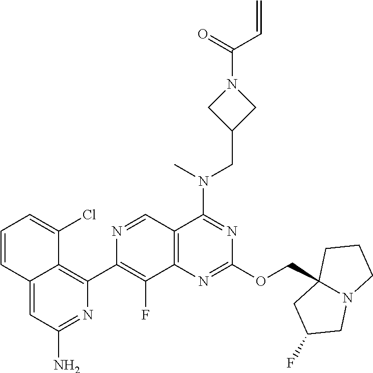 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1] | +++ |
| 74 | 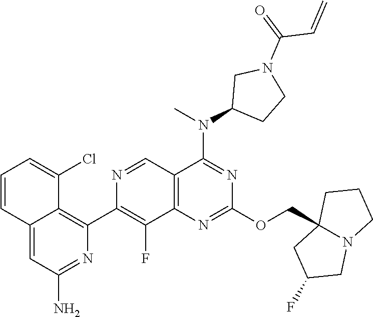 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 75 | 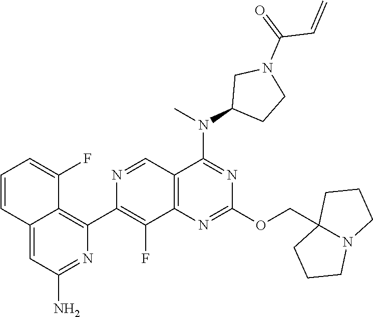 [SMILES: C=CC(=O)N1CC[C@@H](N(C)c2nc(OCC34CCCN3CCC4)nc3c(F)c(-c4nc(N)cc5cccc(F)c45)ncc23)C1] | ++ |
| 76 | 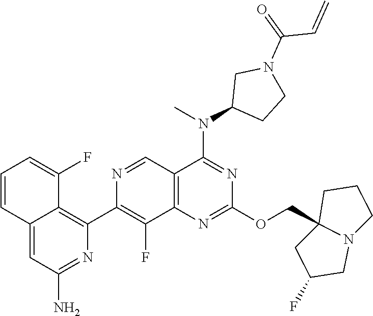 [SMILES: C=CC(=O)N1CC[C@@H](N(C)c2nc(OC[C@@]34CCCN3C[C@H](F)C4)nc3c(F)c(-c4nc(N)cc5cccc(F)c45)ncc23)C1] | +++ |
| 77 (or) pyrrolidine (abs) fluoropyrolizine | 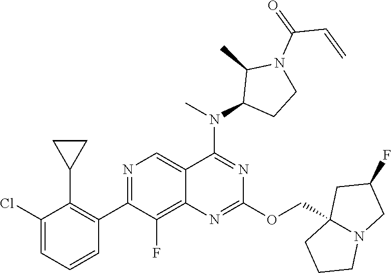 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C] | ++ |
| 78 (or) pyrrolidine (abs) fluoropyrolizine | 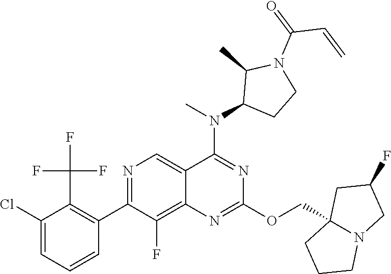 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C] | +++ |
| 79 | 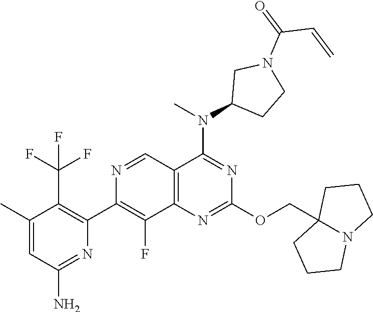 [SMILES: C=CC(=O)N1CC[C@@H](N(C)c2nc(OCC34CCCN3CCC4)nc3c(F)c(-c4nc(N)cc(C)c4C(F)(F)F)ncc23)C1] | +++ |
| 80 | 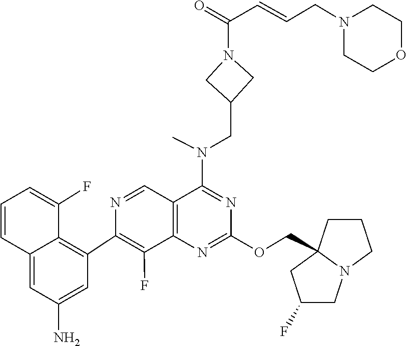 [SMILES: CN(CC1CN(C(=O)/C=C/CN2CCOCC2)C1)c1nc(OC[C@@]23CCCN2C[C@H](F)C3)nc2c(F)c(-c3cc(N)cc4cccc(F)c34)ncc12] | + |
| 81 | 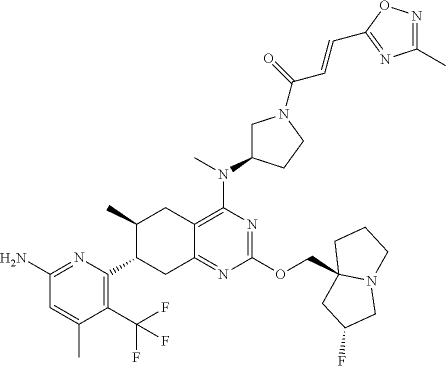 [SMILES: Cc1noc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1] | ++ |
| 82 | 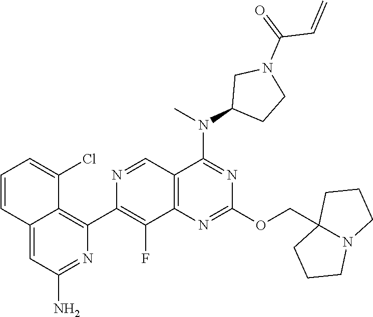 [SMILES: C=CC(=O)N1CC[C@@H](N(C)c2nc(OCC34CCCN3CCC4)nc3c(F)c(-c4nc(N)cc5cccc(Cl)c45)ncc23)C1] | ++ |
| 83 | 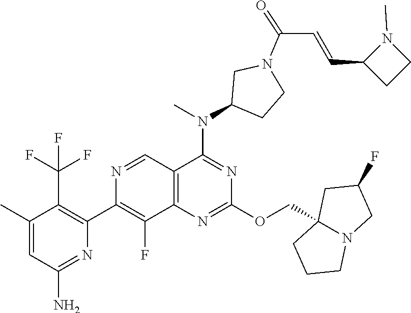 [SMILES: Cc1cc(N)nc(-c2ncc3c(N(C)[C@@H]4CCN(C(=O)/C=C/[C@@H]5CCN5C)C4)nc(OC[C@@]45CCCN4C[C@H](F)C5)nc3c2F)c1C(F)(F)F] | ++++ |
| 84 | 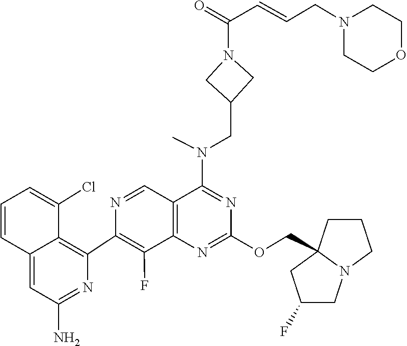 [SMILES: CN(CC1CN(C(=O)/C=C/CN2CCOCC2)C1)c1nc(OC[C@@]23CCCN2C[C@H](F)C3)nc2c(F)c(-c3nc(N)cc4cccc(Cl)c34)ncc12] | +++ |
| 85 | 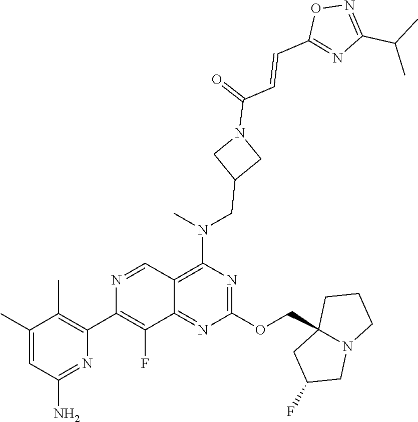 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C] | ++ |
| 86 | 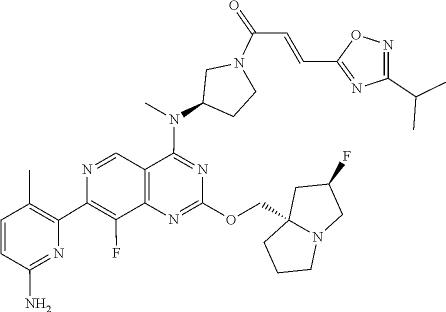 [SMILES: Cc1ccc(N)nc1-c1ncc2c(N(C)[C@@H]3CCN(C(=O)/C=C/c4nc(C(C)C)no4)C3)nc(OC[C@@]34CCCN3C[C@H](F)C4)nc2c1F] | +++ |
| 87 | 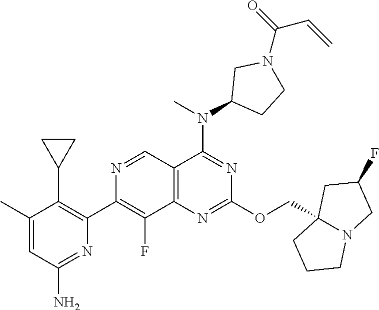 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1] | ++ |
| 88 | 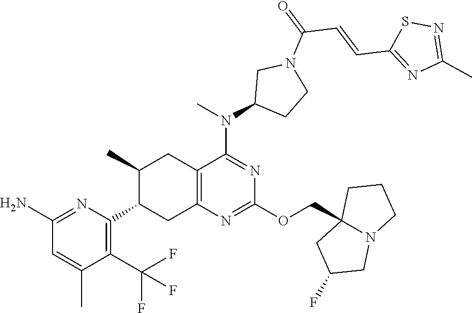 [SMILES: Cc1nsc(/C=C/C(=O)N2CC[C@@H](N(C)c3nc(OC[C@@]45CCCN4C[C@H](F)C5)nc4c3C[C@H](C)[C@@H](c3nc(N)cc(C)c3C(F)(F)F)C4)C2)n1] | + |
| 89 | 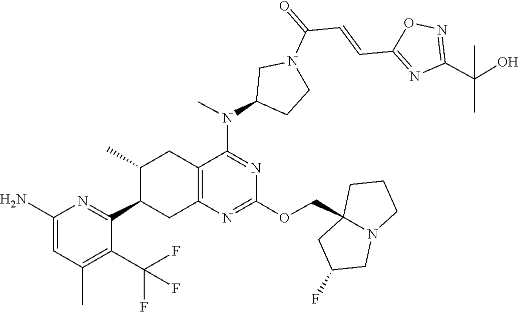 [SMILES: Cc1cc(N)nc([C@@H]2Cc3nc(OC[C@@]45CCCN4C[C@H](F)C5)nc(N(C)[C@@H]4CCN(C(=O)/C=C/c5nc(C(C)(C)O)no5)C4)c3C[C@H]2C)c1C(F)(F)F] | ++++ |
| 90 | 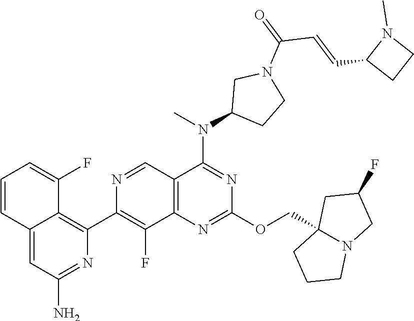 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ++++ |
| 91 | 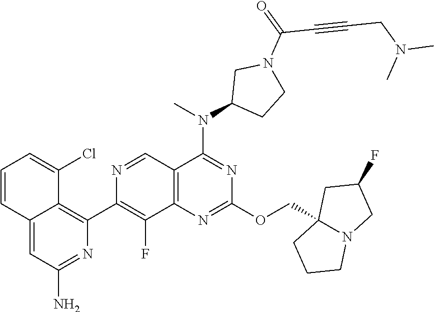 [SMILES: CN(C)CC#CC(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 92 | 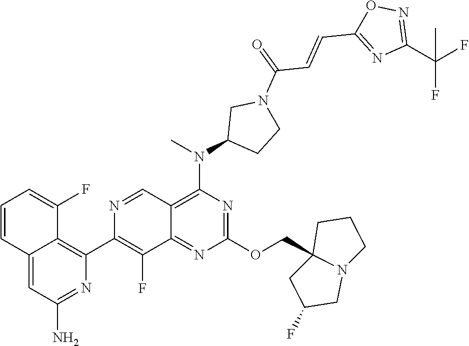 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1] | ++++ |
| 93 | 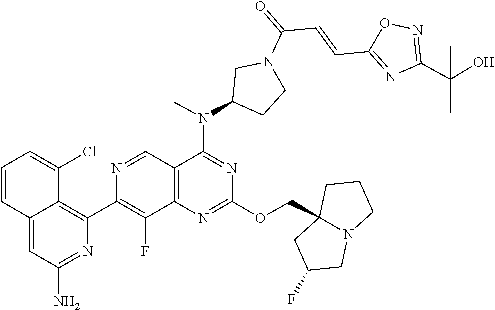 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1] | ++++ |
| 94 | 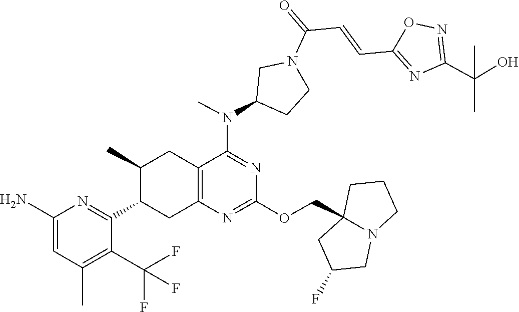 [SMILES: Cc1cc(N)nc([C@H]2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F] | + |
| 95 | 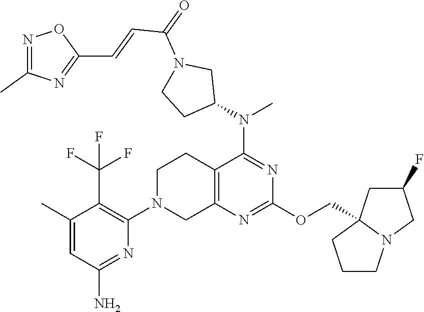 [SMILES: Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1] | + |
| 96 | 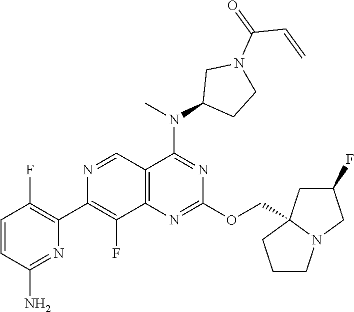 [SMILES: C=CC(=O)N1CC[C@@H](N(C)c2nc(OC[C@@]34CCCN3C[C@H](F)C4)nc3c(F)c(-c4nc(N)ccc4F)ncc23)C1] | + |
| 97 | 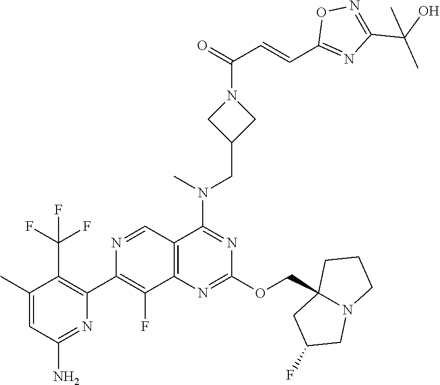 [SMILES: Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F] | +++ |
| 98 | 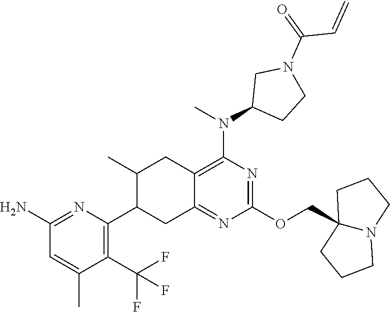 | ++ |
| 99 | 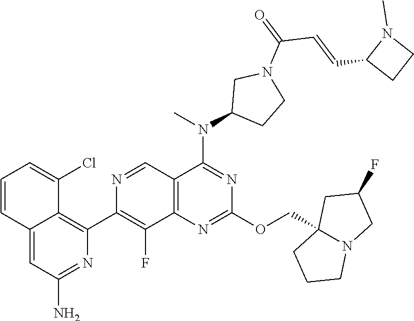 [SMILES: CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | ++++ |
| 100 | 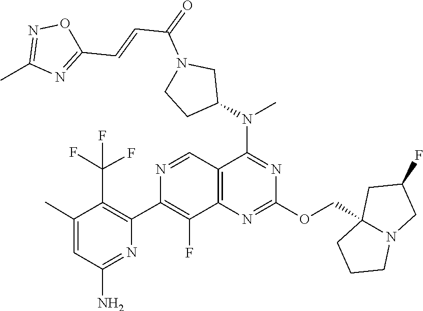 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1] | ++++ |
| 101 | 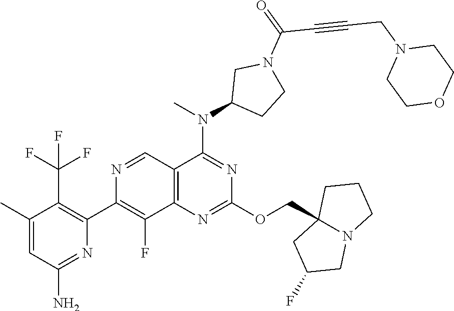 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F] | +++ |
| 102 | 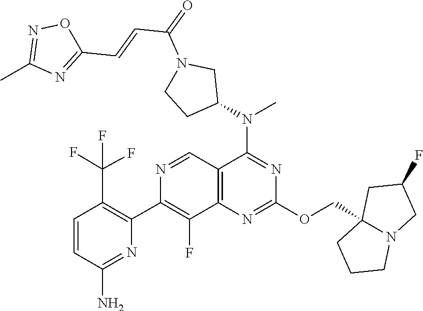 [SMILES: Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1] | +++ |
| 103 | 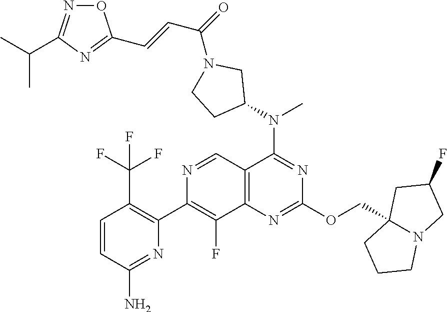 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1] | +++ |
| 104 | 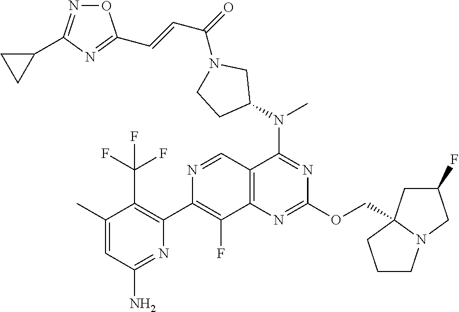 [SMILES: Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F] | ++++ |
| 105 | 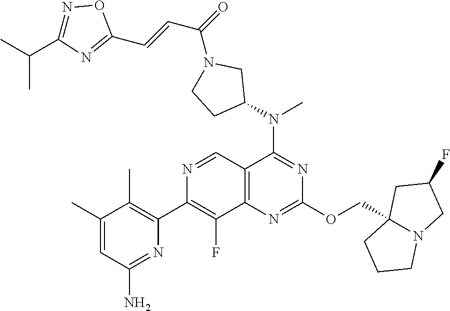 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C] | +++ |
| 106 | 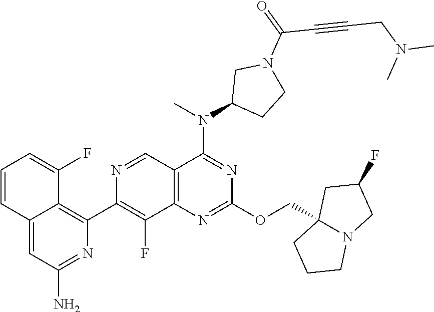 [SMILES: CN(C)CC#CC(=O)N1CC[C@@H](N(C)c2nc(OC[C@@]34CCCN3C[C@H](F)C4)nc3c(F)c(-c4nc(N)cc5cccc(F)c45)ncc23)C1] | ++++ |
| 107 | 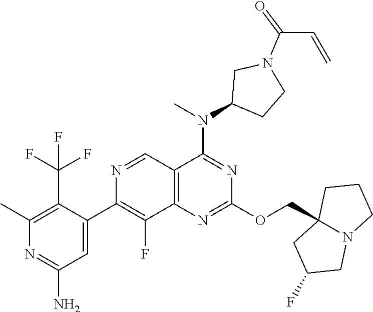 [SMILES: C=CC(=O)N1CC[C@@H](N(C)c2nc(OC[C@@]34CCCN3C[C@H](F)C4)nc3c(F)c(-c4cc(N)nc(C)c4C(F)(F)F)ncc23)C1] | +++ |
| 108 | 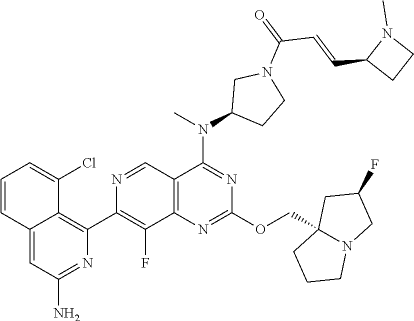 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 109 | 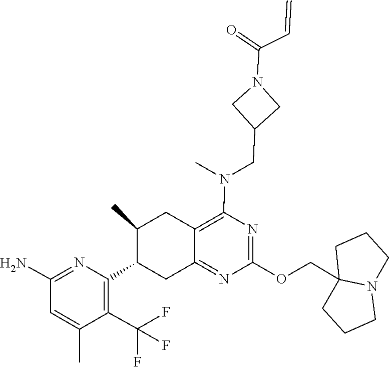 [SMILES: C=CC(=O)N1CC(CN(C)c2nc(OCC34CCCN3CCC4)nc3c2C[C@H](C)[C@@H](c2nc(N)cc(C)c2C(F)(F)F)C3)C1] | + |
| 110 | 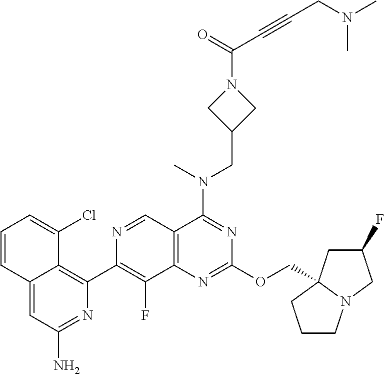 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1] | +++ |
| 111 | 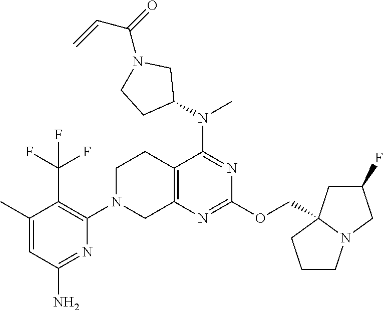 [SMILES: C=CC(=O)N1CC[C@@H](N(C)c2nc(OC[C@@]34CCCN3C[C@H](F)C4)nc3c2CCN(c2nc(N)cc(C)c2C(F)(F)F)C3)C1] | + |
| 112 | 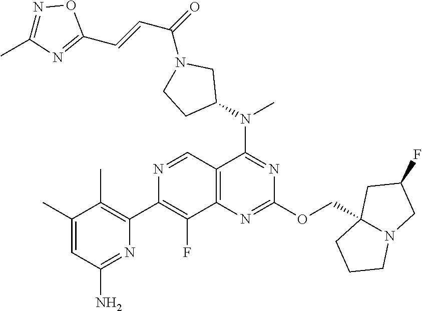 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1] | +++ |
| 113 | 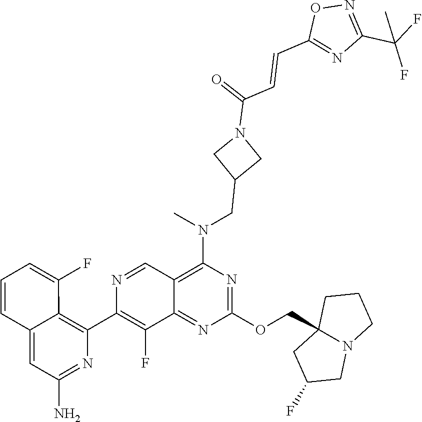 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12] | ++++ |
| 114 | 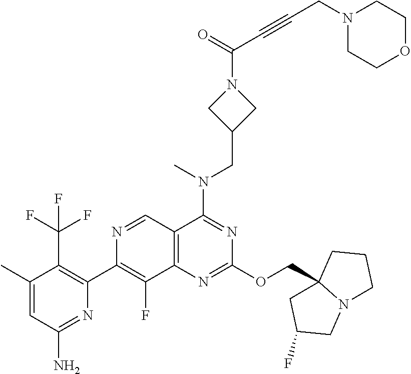 [SMILES: Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F] | +++ |
| 115 | 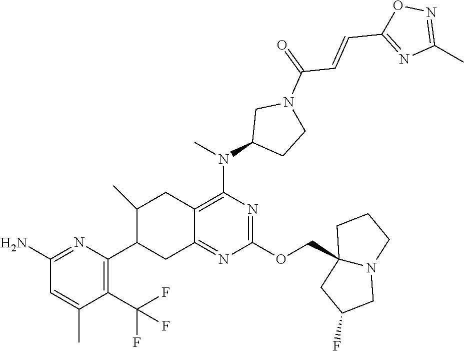 [SMILES: Cc1noc(/C=C/C(=O)N2CC[C@@H](N(C)c3nc(OC[C@@]45CCCN4C[C@H](F)C5)nc4c3CC(C)C(c3nc(N)cc(C)c3C(F)(F)F)C4)C2)n1] | ++++ |
| 116 | 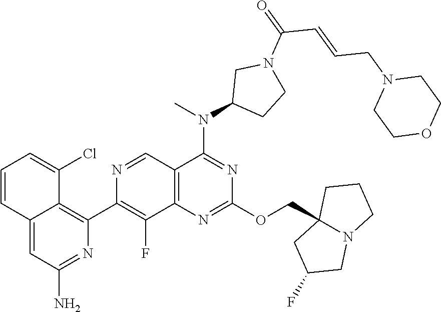 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/CN2CCOCC2)C1] | ++ |
| 117 | 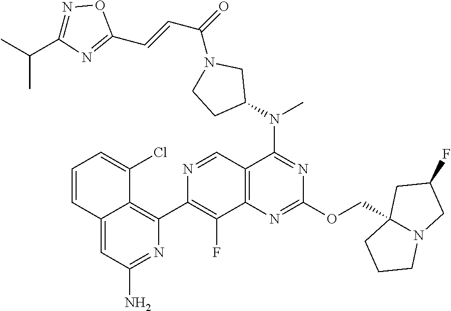 [SMILES: CC(C)c1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1] | ++++ |
| 118 | 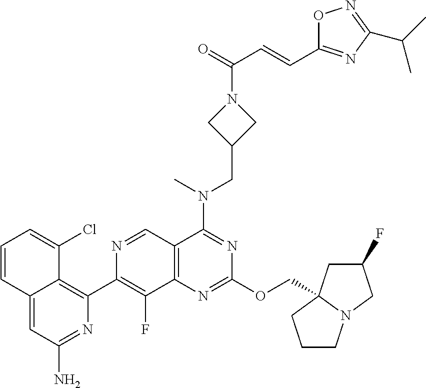 [SMILES: CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1] | ++ |
| 119 | 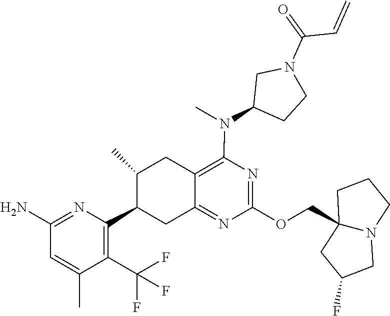 [SMILES: C=CC(=O)N1CC[C@@H](N(C)c2nc(OC[C@@]34CCCN3C[C@H](F)C4)nc3c2C[C@@H](C)[C@H](c2nc(N)cc(C)c2C(F)(F)F)C3)C1] | ++ |
| 120 | 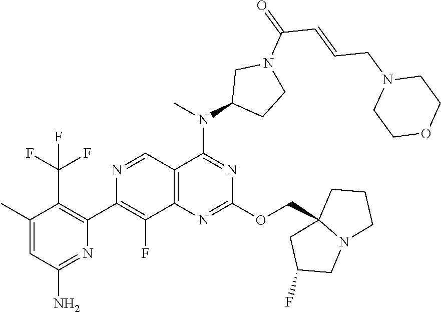 [SMILES: Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F] | ++ |
| 121 | 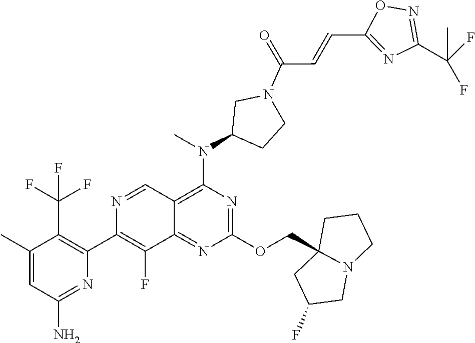 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F] | ++++ |
| 122 | 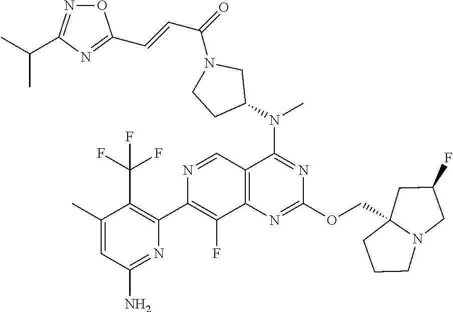 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F] | ++++ |
| 123 | 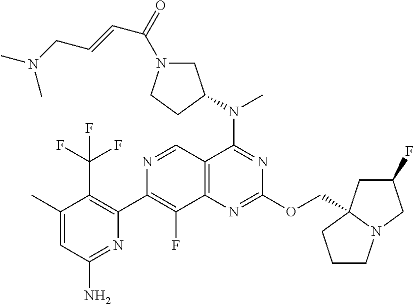 [SMILES: Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F] | +++ |
| 124 | 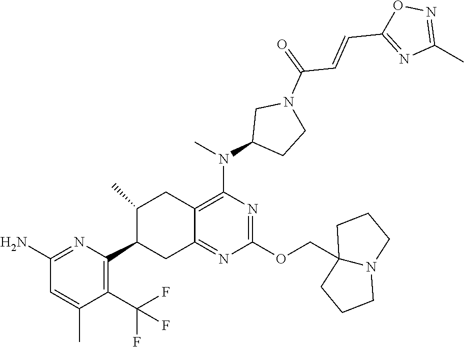 [SMILES: Cc1noc(/C=C/C(=O)N2CC[C@@H](N(C)c3nc(OCC45CCCN4CCC5)nc4c3C[C@@H](C)[C@H](c3nc(N)cc(C)c3C(F)(F)F)C4)C2)n1] | ++ |
| 125 | 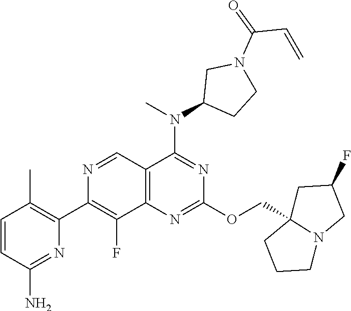 [SMILES: C=CC(=O)N1CC[C@@H](N(C)c2nc(OC[C@@]34CCCN3C[C@H](F)C4)nc3c(F)c(-c4nc(N)ccc4C)ncc23)C1] | + |
| 126 | 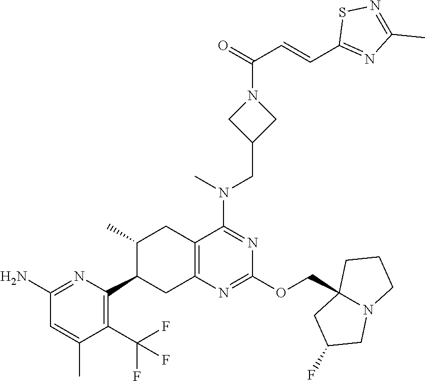 [SMILES: Cc1nsc(/C=C/C(=O)N2CC(CN(C)c3nc(OC[C@@]45CCCN4C[C@H](F)C5)nc4c3C[C@@H](C)[C@H](c3nc(N)cc(C)c3C(F)(F)F)C4)C2)n1] | +++ |
| 128 | 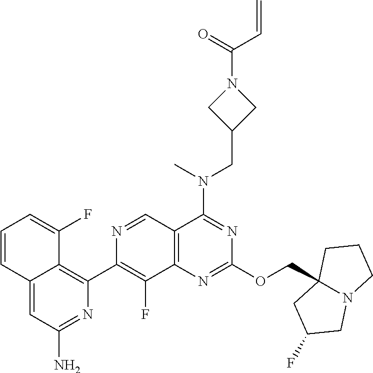 [SMILES: C=CC(=O)N1CC(CN(C)c2nc(OC[C@@]34CCCN3C[C@H](F)C4)nc3c(F)c(-c4nc(N)cc5cccc(F)c45)ncc23)C1] | + |
| 129 | 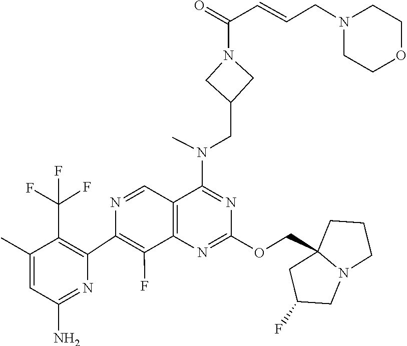 [SMILES: Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F] | ++ |
| 130 | 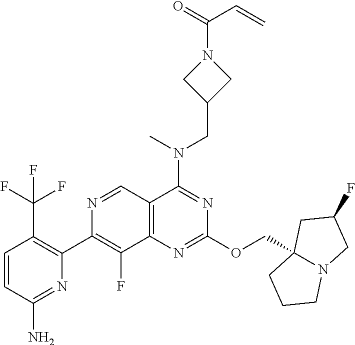 [SMILES: C=CC(=O)N1CC(CN(C)c2nc(OC[C@@]34CCCN3C[C@H](F)C4)nc3c(F)c(-c4nc(N)ccc4C(F)(F)F)ncc23)C1] | ++ |
| 131 | 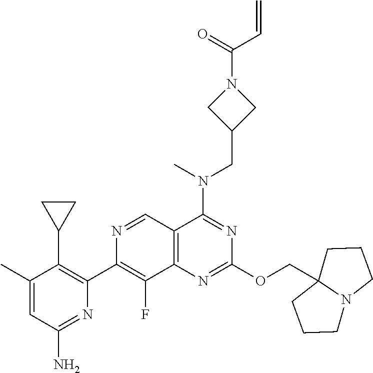 [SMILES: C=CC(=O)N1CC(CN(C)c2nc(OCC34CCCN3CCC4)nc3c(F)c(-c4nc(N)cc(C)c4C4CC4)ncc23)C1] | + |
| 132 | 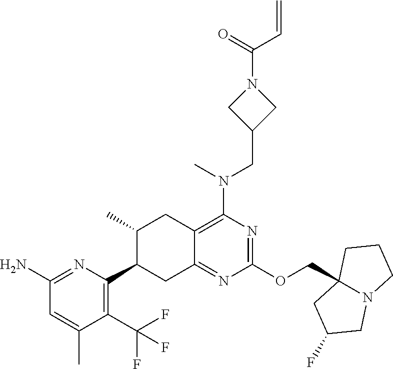 [SMILES: C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1] | ++ |
| 133 | 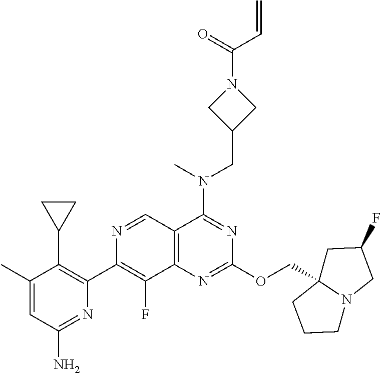 [SMILES: C=CC(=O)N1CC(CN(C)c2nc(OC[C@@]34CCCN3C[C@H](F)C4)nc3c(F)c(-c4nc(N)cc(C)c4C4CC4)ncc23)C1] | + |
| 134 | 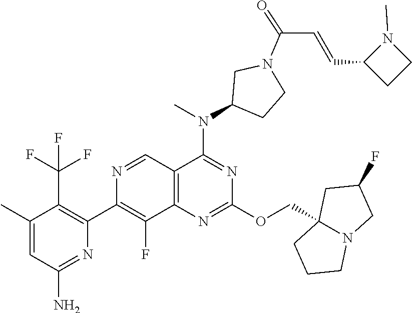 [SMILES: Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F] | ++++ |
| 135 | 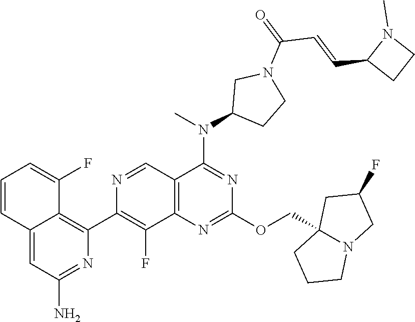 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1] | ++ |
| 136 | 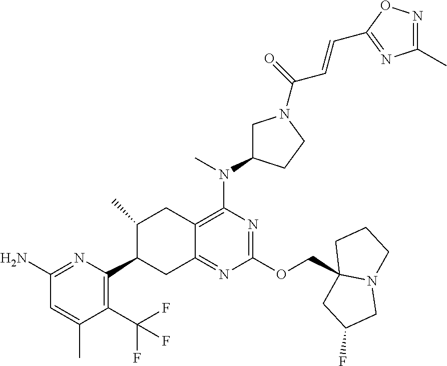 [SMILES: Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1] | ++++ |
| 137 | 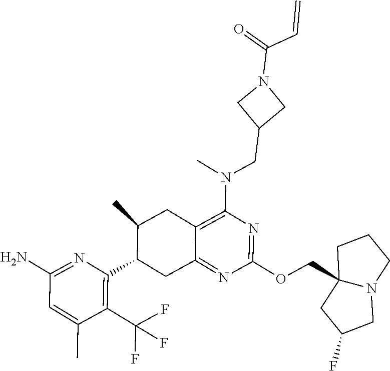 [SMILES: C=CC(=O)N1CC(CN(C)c2nc(OC[C@@]34CCCN3C[C@H](F)C4)nc3c2C[C@H](C)[C@@H](c2nc(N)cc(C)c2C(F)(F)F)C3)C1] | + |
| 138 | 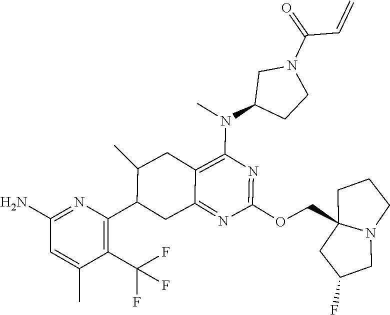 [SMILES: C=CC(=O)N1CC[C@@H](N(C)c2nc(OC[C@@]34CCCN3C[C@H](F)C4)nc3c2CC(C)C(c2nc(N)cc(C)c2C(F)(F)F)C3)C1] | ++ |
| 139 | 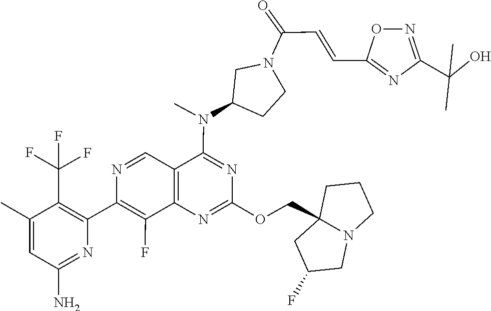 [SMILES: Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F] | ++++ |
| 140 | 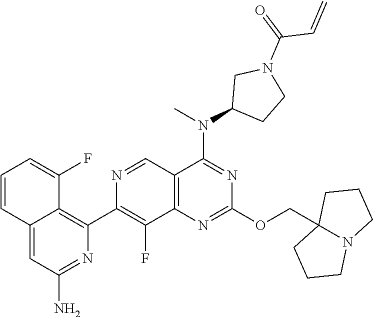 [SMILES: C=CC(=O)N1CC[C@@H](N(C)c2nc(OCC34CCCN3CCC4)nc3c(F)c(-c4nc(N)cc5cccc(F)c45)ncc23)C1] | ++ |
| 141 | 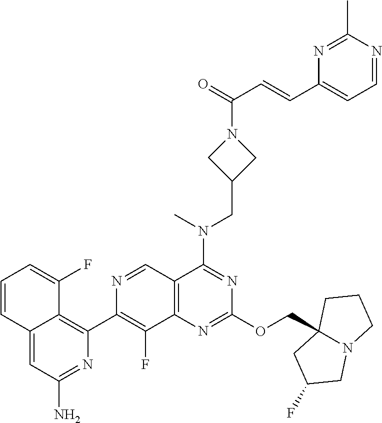 [SMILES: Cc1nccc(/C=C/C(=O)N2CC(CN(C)c3nc(OC[C@@]45CCCN4C[C@H](F)C5)nc4c(F)c(-c5nc(N)cc6cccc(F)c56)ncc34)C2)n1] | +++ |
| 142 | 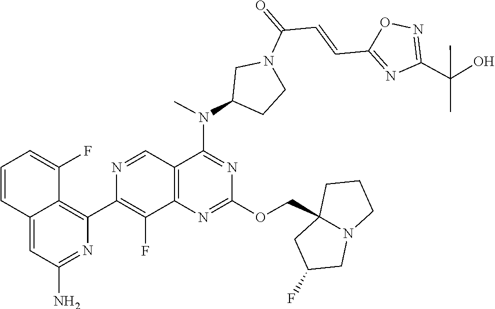 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1] | ++++ |
| 143 | 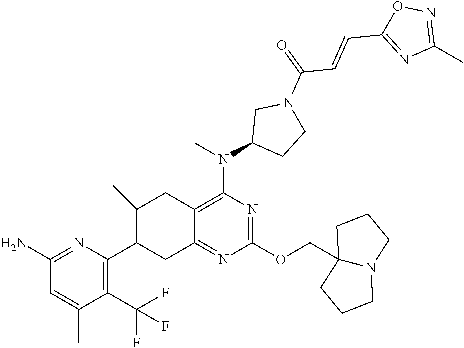 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1] | ++++ |
| 144 | 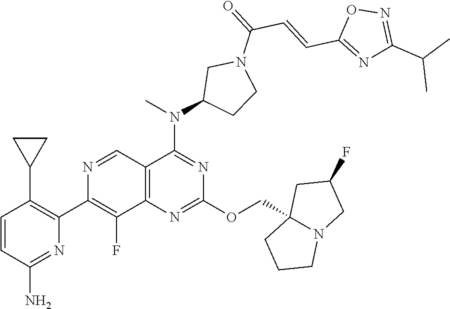 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1] | +++ |
| 145 | 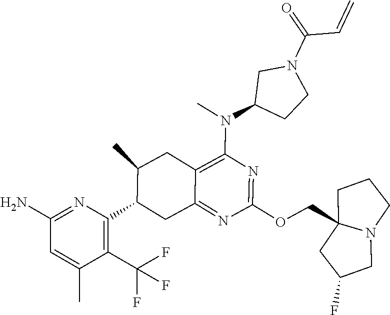 [SMILES: C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1] | + |
| 146 | 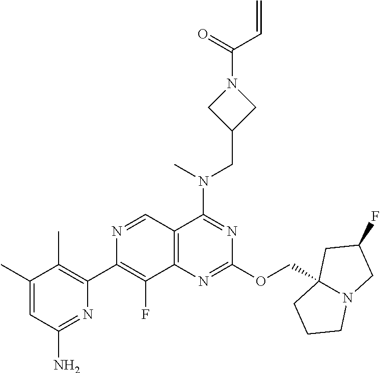 [SMILES: C=CC(=O)N1CC(CN(C)c2nc(OC[C@@]34CCCN3C[C@H](F)C4)nc3c(F)c(-c4nc(N)cc(C)c4C)ncc23)C1] | + |
| 147 | 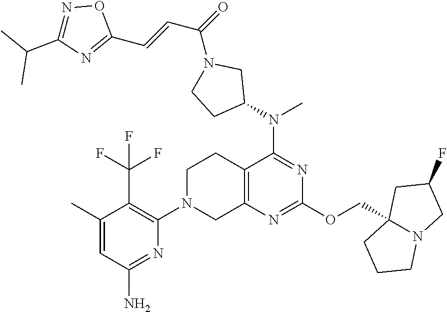 [SMILES: Cc1cc(N)nc(N2CCc3c(nc(OC[C@@]45CCCN4C[C@H](F)C5)nc3N(C)[C@@H]3CCN(C(=O)/C=C/c4nc(C(C)C)no4)C3)C2)c1C(F)(F)F] | + |
| 148 | 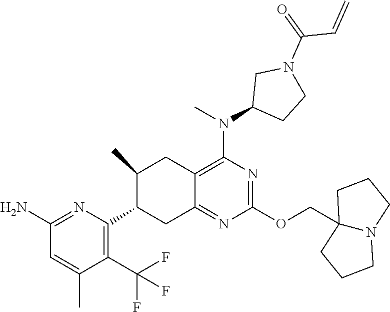 [SMILES: C=CC(=O)N1CC[C@@H](N(C)c2nc(OCC34CCCN3CCC4)nc3c2C[C@H](C)[C@@H](c2nc(N)cc(C)c2C(F)(F)F)C3)C1] | ++ |
| 149 | 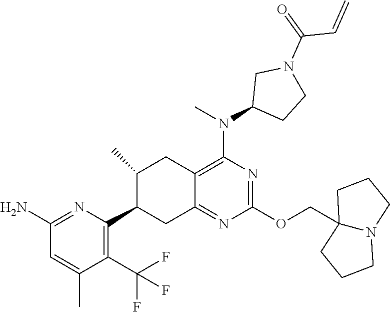 [SMILES: C=CC(=O)N1CC[C@@H](N(C)c2nc(OCC34CCCN3CCC4)nc3c2C[C@@H](C)[C@H](c2nc(N)cc(C)c2C(F)(F)F)C3)C1] | + |
| 150 | 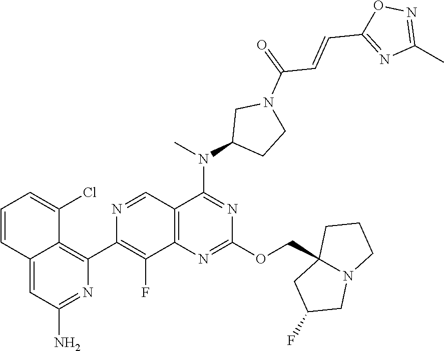 [SMILES: Cc1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1] | ++++ |
| 151 | 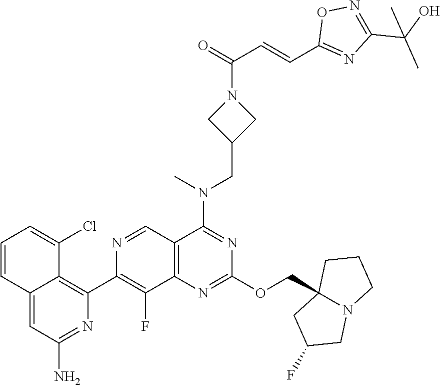 [SMILES: CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12] | ++++ |
| 152 | 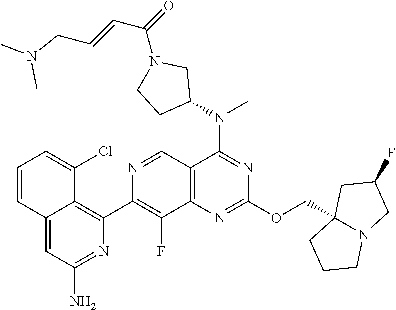 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 153 | 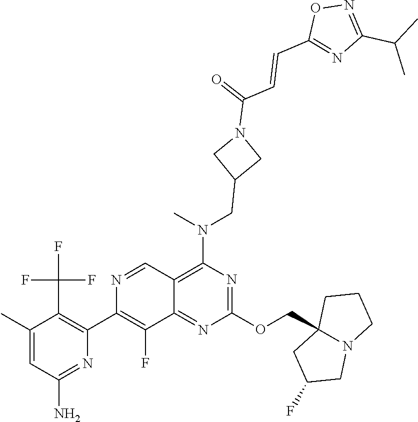 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C(F)(F)F] | ++++ |
| 154 | 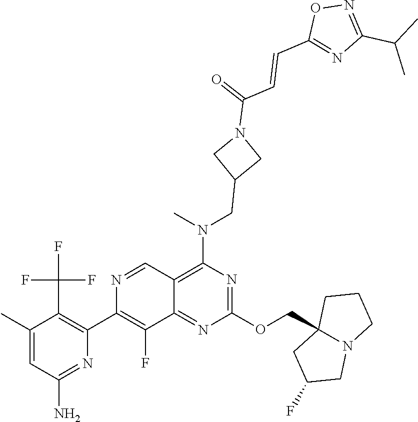 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C(F)(F)F] | +++ |
| 155 | 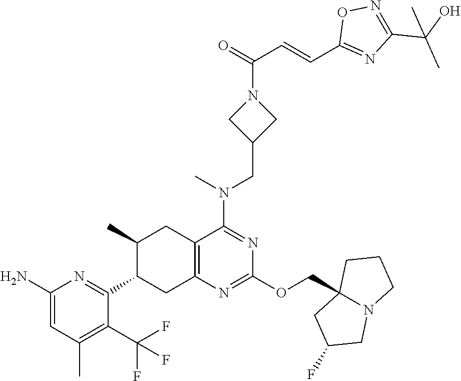 [SMILES: Cc1cc(N)nc([C@H]2Cc3nc(OC[C@@]45CCCN4C[C@H](F)C5)nc(N(C)CC4CN(C(=O)/C=C/c5nc(C(C)(C)O)no5)C4)c3C[C@@H]2C)c1C(F)(F)F] | + |
| 156 | 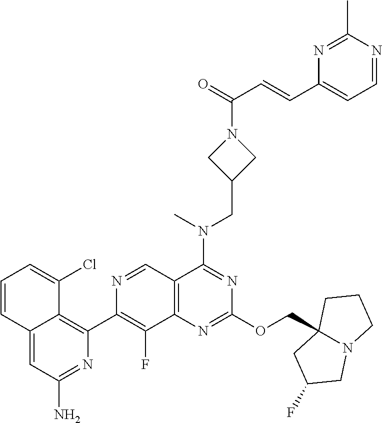 [SMILES: Cc1nccc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1] | +++ |
| 157 | 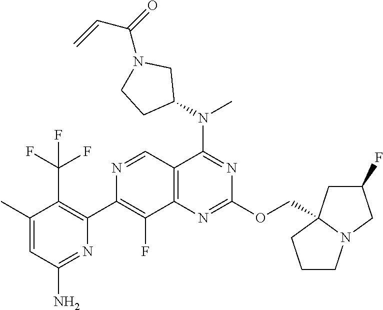 [SMILES: C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1] | +++ |
| 158 | 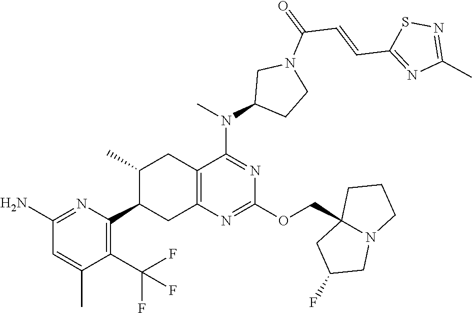 [SMILES: Cc1nsc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1] | +++ |
| 159 | 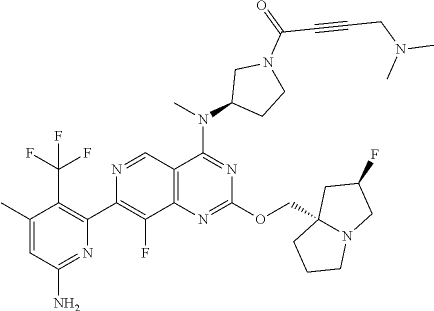 | ++++ |
| 160 | 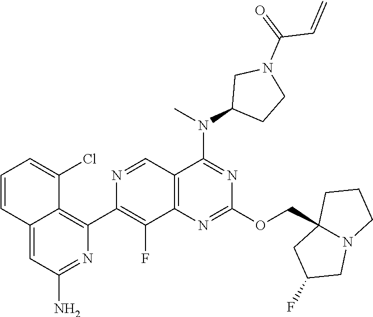 [SMILES: C=CC(=O)N1CC[C@@H](N(C)c2nc(OC[C@@]34CCCN3C[C@H](F)C4)nc3c(F)c(-c4nc(N)cc5cccc(Cl)c45)ncc23)C1] | +++ |
| 161 | 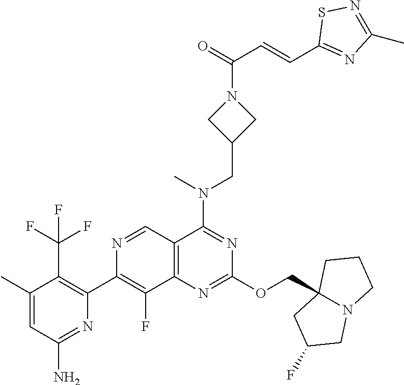 | +++ |
| 162 | 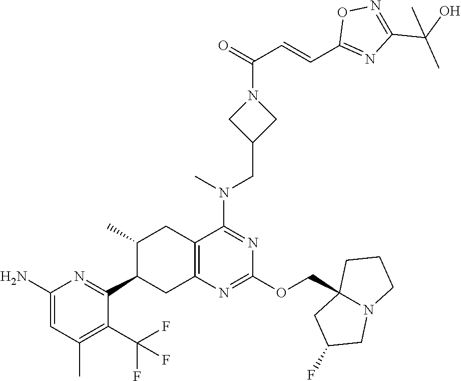 [SMILES: Cc1cc(N)nc([C@@H]2Cc3nc(OC[C@@]45CCCN4C[C@H](F)C5)nc(N(C)CC4CN(C(=O)/C=C/c5nc(C(C)(C)O)no5)C4)c3C[C@H]2C)c1C(F)(F)F] | +++ |
| 163 | 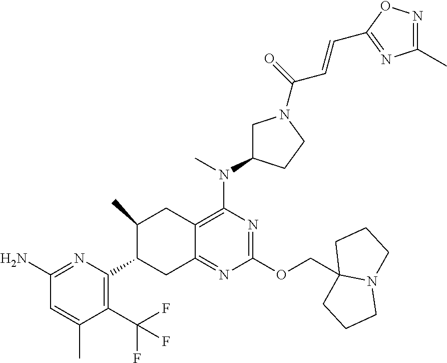 [SMILES: Cc1noc(/C=C/C(=O)N2CC[C@@H](N(C)c3nc(OCC45CCCN4CCC5)nc4c3C[C@H](C)[C@@H](c3nc(N)cc(C)c3C(F)(F)F)C4)C2)n1] | +++ |
| 164 | 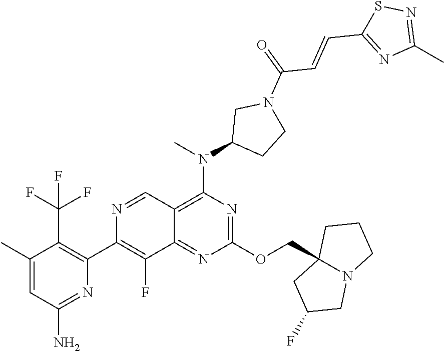 | +++ |
| 165 | 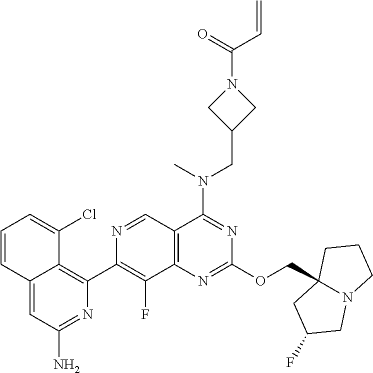 [SMILES: C=CC(=O)N1CC(CN(C)c2nc(OC[C@@]34CCCN3C[C@H](F)C4)nc3c(F)c(-c4nc(N)cc5cccc(Cl)c45)ncc23)C1] | +++ |
| 166 | 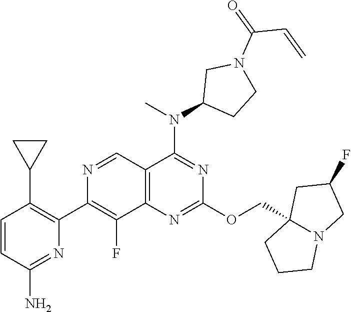 [SMILES: C=CC(=O)N1CC[C@@H](N(C)c2nc(OC[C@@]34CCCN3C[C@H](F)C4)nc3c(F)c(-c4nc(N)ccc4C4CC4)ncc23)C1] | + |
| 167 | 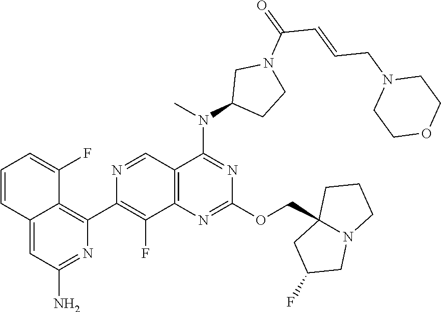 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1] | + |
| 168 | 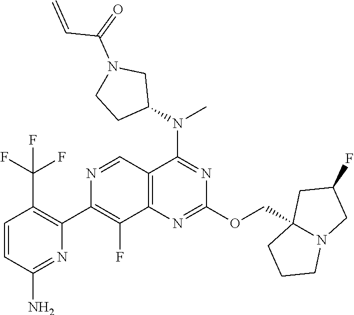 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1] | ++ |
| 169 | 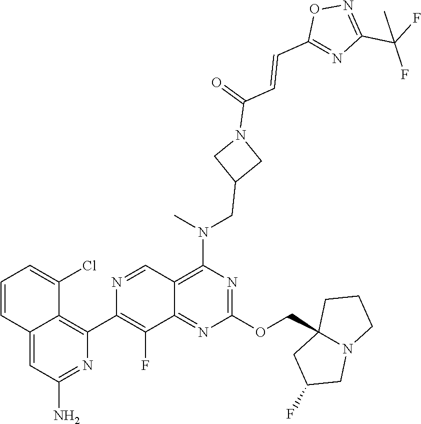 [SMILES: CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12] | ++++ |
| 170 | 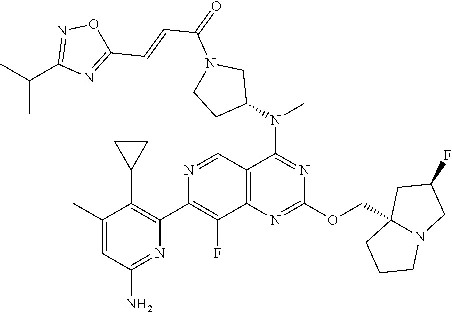 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1] | +++ |
| 171 | 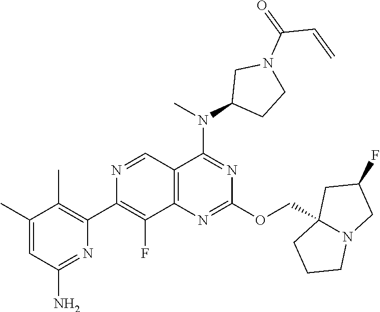 [SMILES: C=CC(=O)N1CC[C@@H](N(C)c2nc(OC[C@@]34CCCN3C[C@H](F)C4)nc3c(F)c(-c4nc(N)cc(C)c4C)ncc23)C1] | ++ |
| 172 | 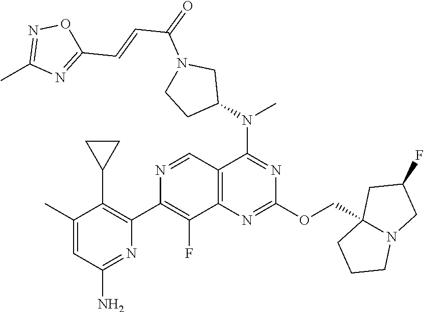 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1] | ++++ |
| 173 | 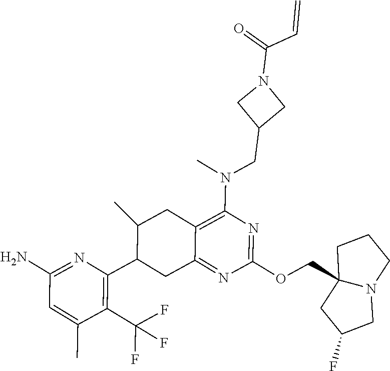 [SMILES: C=CC(=O)N1CC(CN(C)c2nc(OC[C@@]34CCCN3C[C@H](F)C4)nc3c2CC(C)C(c2nc(N)cc(C)c2C(F)(F)F)C3)C1] | + |
| 174 | 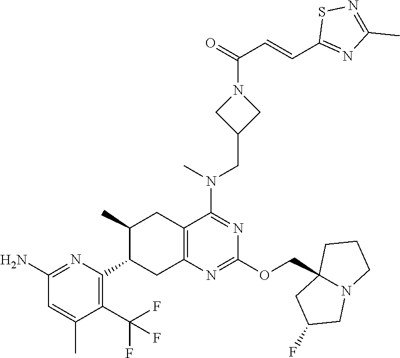 [SMILES: Cc1nsc(/C=C/C(=O)N2CC(CN(C)c3nc(OC[C@@]45CCCN4C[C@H](F)C5)nc4c3C[C@H](C)[C@@H](c3nc(N)cc(C)c3C(F)(F)F)C4)C2)n1] | + |
| 175 | 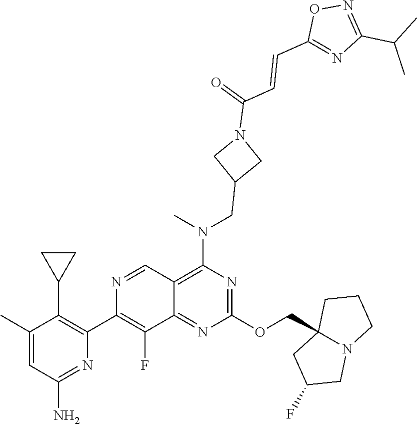 [SMILES: Cc1cc(N)nc(-c2ncc3c(N(C)CC4CN(C(=O)/C=C/c5nc(C(C)C)no5)C4)nc(OC[C@@]45CCCN4C[C@H](F)C5)nc3c2F)c1C1CC1] | ++ |
| 176 | 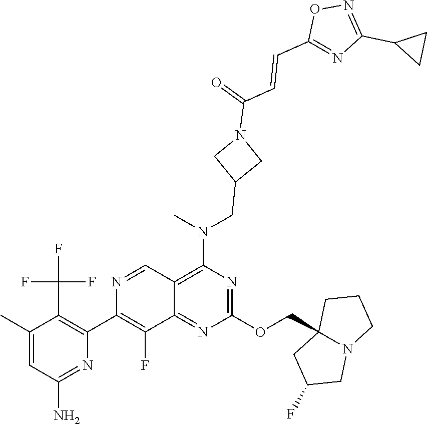 [SMILES: Cc1cc(N)nc(-c2ncc3c(N(C)CC4CN(C(=O)/C=C/c5nc(C6CC6)no5)C4)nc(OC[C@@]45CCCN4C[C@H](F)C5)nc3c2F)c1C(F)(F)F] | ++++ |
| 177 | 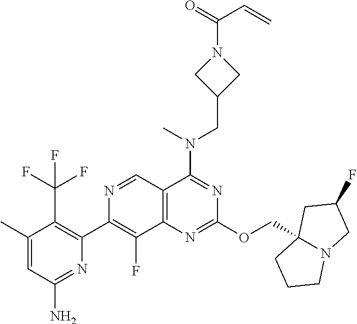 [SMILES: C=CC(=O)N1CC(CN(C)c2nc(OC[C@@]34CCCN3C[C@H](F)C4)nc3c(F)c(-c4nc(N)cc(C)c4C(F)(F)F)ncc23)C1] | +++ |
| 178 | 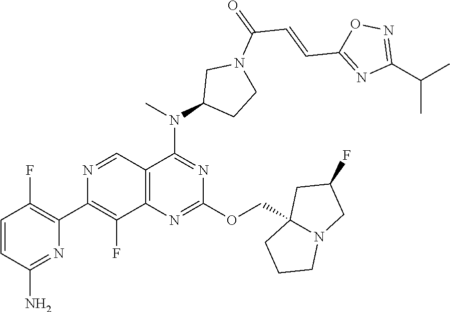 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5F)ncc34)C2)n1] | + |
| 179 | 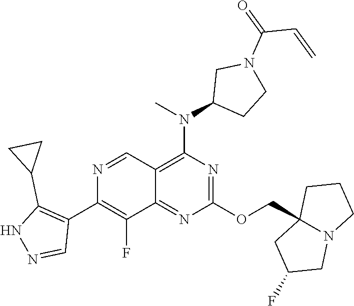 [SMILES: C=CC(=O)N1CC[C@@H](N(C)c2nc(OC[C@@]34CCCN3C[C@H](F)C4)nc3c(F)c(-c4cn[nH]c4C4CC4)ncc23)C1] | + |
| 180 | 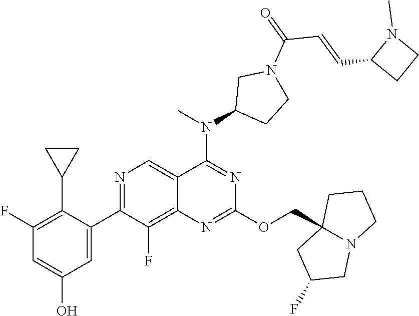 [SMILES: CN1CC[C@@H]1/C=C/C(=O)N1CC[C@@H](N(C)c2nc(OC[C@@]34CCCN3C[C@H](F)C4)nc3c(F)c(-c4cc(O)cc(F)c4C4CC4)ncc23)C1] | ++++ |
| 181 | 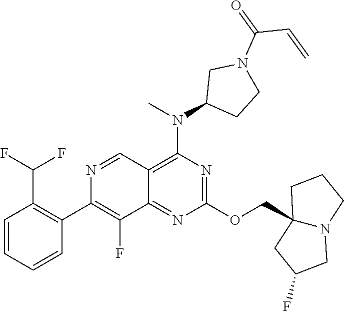 [SMILES: C=CC(=O)N1CC[C@@H](N(C)c2nc(OC[C@@]34CCCN3C[C@H](F)C4)nc3c(F)c(-c4ccccc4C(F)F)ncc23)C1] | + |
| 182 | 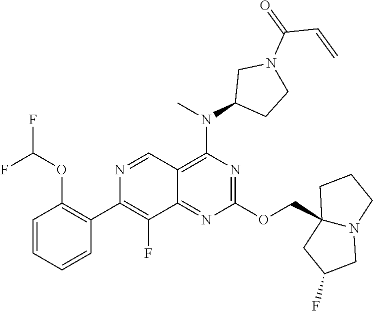 [SMILES: C=CC(=O)N1CC[C@@H](N(C)c2nc(OC[C@@]34CCCN3C[C@H](F)C4)nc3c(F)c(-c4ccccc4OC(F)F)ncc23)C1] | + |
| 183 | 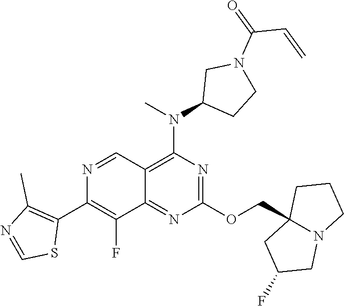 [SMILES: C=CC(=O)N1CC[C@@H](N(C)c2nc(OC[C@@]34CCCN3C[C@H](F)C4)nc3c(F)c(-c4scnc4C)ncc23)C1] | + |
| 184 | 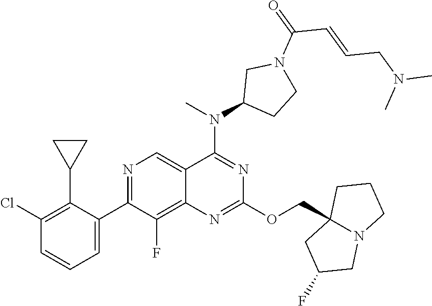 [SMILES: CN(C)C/C=C/C(=O)N1CC[C@@H](N(C)c2nc(OC[C@@]34CCCN3C[C@H](F)C4)nc3c(F)c(-c4cccc(Cl)c4C4CC4)ncc23)C1] | +++ |
| 185 | 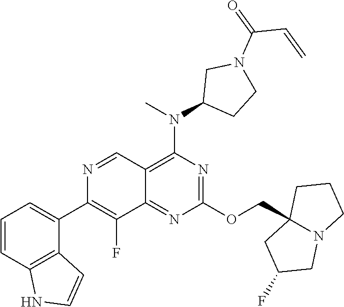 [SMILES: C=CC(=O)N1CC[C@@H](N(C)c2nc(OC[C@@]34CCCN3C[C@H](F)C4)nc3c(F)c(-c4cccc5[nH]ccc45)ncc23)C1] | |
| 186 | 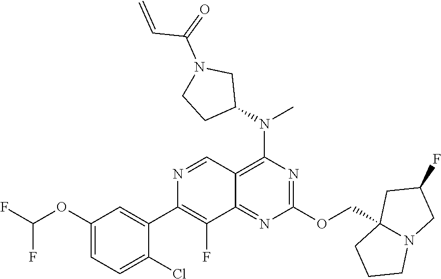 [SMILES: C=CC(=O)N1CC[C@@H](N(C)c2nc(OC[C@@]34CCCN3C[C@H](F)C4)nc3c(F)c(-c4cc(OC(F)F)ccc4Cl)ncc23)C1] | + |
| 187 | 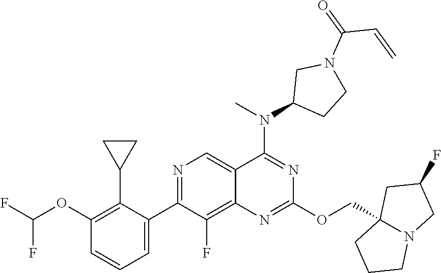 [SMILES: C=CC(=O)N1CC[C@@H](N(C)c2nc(OC[C@@]34CCCN3C[C@H](F)C4)nc3c(F)c(-c4cccc(OC(F)F)c4C4CC4)ncc23)C1] | + |
| 188 | 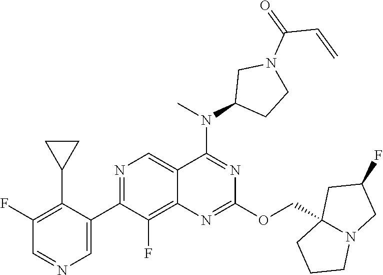 [SMILES: C=CC(=O)N1CC[C@@H](N(C)c2nc(OC[C@@]34CCCN3C[C@H](F)C4)nc3c(F)c(-c4cncc(F)c4C4CC4)ncc23)C1] | + |
| 189 | 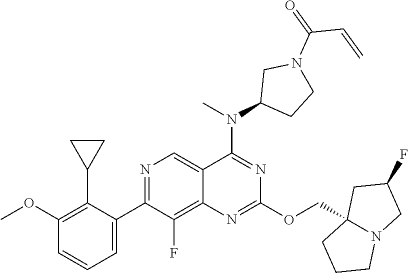 [SMILES: C=CC(=O)N1CC[C@@H](N(C)c2nc(OC[C@@]34CCCN3C[C@H](F)C4)nc3c(F)c(-c4cccc(OC)c4C4CC4)ncc23)C1] | + |
| 190 | 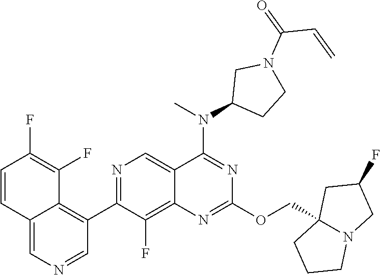 [SMILES: C=CC(=O)N1CC[C@@H](N(C)c2nc(OC[C@@]34CCCN3C[C@H](F)C4)nc3c(F)c(-c4cncc5ccc(F)c(F)c45)ncc23)C1] | + |
| 191 | 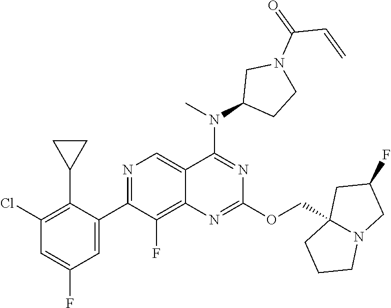 [SMILES: C=CC(=O)N1CC[C@@H](N(C)c2nc(OC[C@@]34CCCN3C[C@H](F)C4)nc3c(F)c(-c4cc(F)cc(Cl)c4C4CC4)ncc23)C1] | ++ |
| 192 | 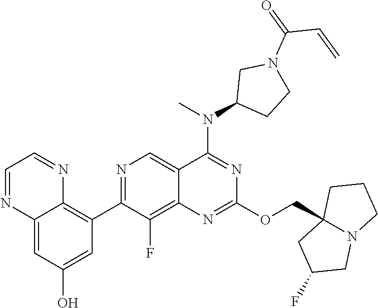 [SMILES: C=CC(=O)N1CC[C@@H](N(C)c2nc(OC[C@@]34CCCN3C[C@H](F)C4)nc3c(F)c(-c4cc(O)cc5nccnc45)ncc23)C1] | ++ |
| 193 | 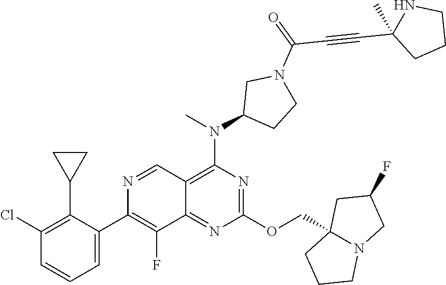 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@]2(C)CCCN2)C1] | ++++ |
| 194 | 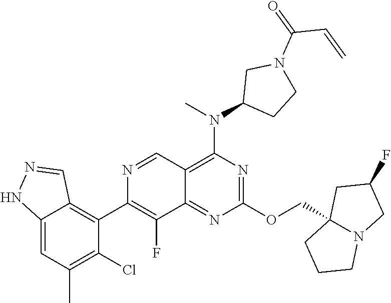 [SMILES: C=CC(=O)N1CC[C@@H](N(C)c2nc(OC[C@@]34CCCN3C[C@H](F)C4)nc3c(F)c(-c4c(Cl)c(C)cc5[nH]ncc45)ncc23)C1] | +++ |
| 195 | 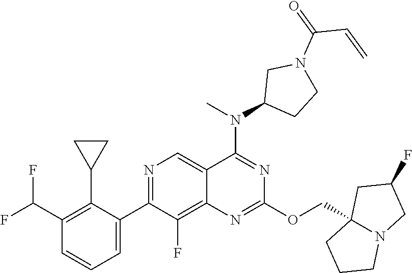 [SMILES: C=CC(=O)N1CC[C@@H](N(C)c2nc(OC[C@@]34CCCN3C[C@H](F)C4)nc3c(F)c(-c4cccc(C(F)F)c4C4CC4)ncc23)C1] | + |
| 196 | 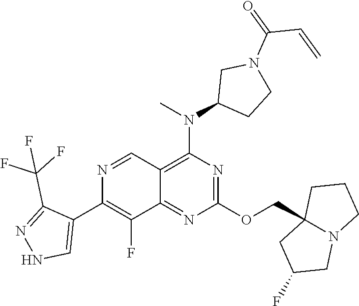 [SMILES: C=CC(=O)N1CC[C@@H](N(C)c2nc(OC[C@@]34CCCN3C[C@H](F)C4)nc3c(F)c(-c4c[nH]nc4C(F)(F)F)ncc23)C1] | + |
| 197 | 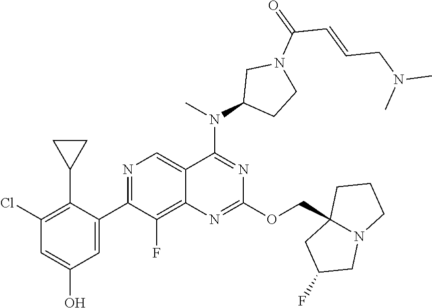 [SMILES: CN(C)C/C=C/C(=O)N1CC[C@@H](N(C)c2nc(OC[C@@]34CCCN3C[C@H](F)C4)nc3c(F)c(-c4cc(O)cc(Cl)c4C4CC4)ncc23)C1] | ++++ |
| 198 | 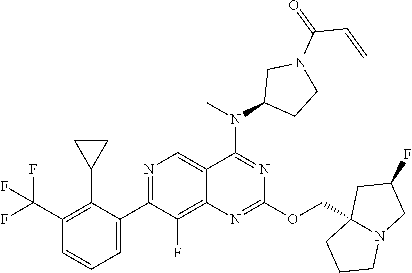 [SMILES: C=CC(=O)N1CC[C@@H](N(C)c2nc(OC[C@@]34CCCN3C[C@H](F)C4)nc3c(F)c(-c4cccc(C(F)(F)F)c4C4CC4)ncc23)C1] | + |
| 199 | 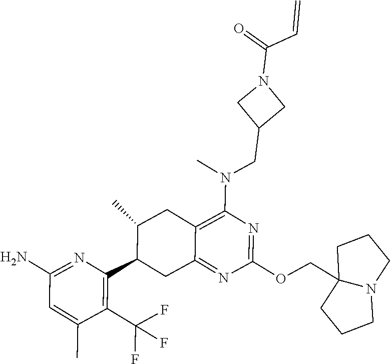 [SMILES: C=CC(=O)N1CC(CN(C)c2nc(OCC34CCCN3CCC4)nc3c2C[C@@H](C)[C@H](c2nc(N)cc(C)c2C(F)(F)F)C3)C1] | + |
| 200 | 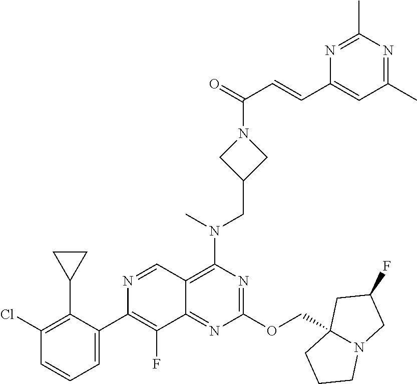 [SMILES: Cc1cc(/C=C/C(=O)N2CC(CN(C)c3nc(OC[C@@]45CCCN4C[C@H](F)C5)nc4c(F)c(-c5cccc(Cl)c5C5CC5)ncc34)C2)nc(C)n1] | ++ |
| 201 | 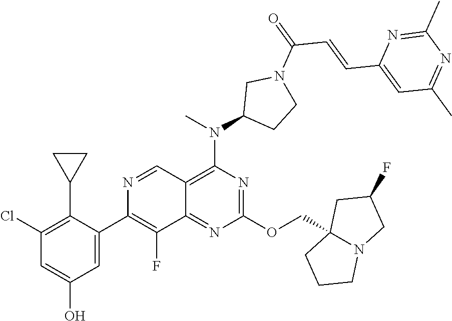 [SMILES: Cc1cc(/C=C/C(=O)N2CC[C@@H](N(C)c3nc(OC[C@@]45CCCN4C[C@H](F)C5)nc4c(F)c(-c5cc(O)cc(Cl)c5C5CC5)ncc34)C2)nc(C)n1] | ++++ |
| 202 | 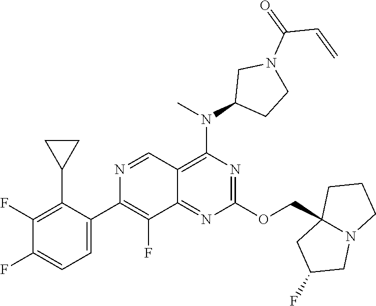 [SMILES: C=CC(=O)N1CC[C@@H](N(C)c2nc(OC[C@@]34CCCN3C[C@H](F)C4)nc3c(F)c(-c4ccc(F)c(F)c4C4CC4)ncc23)C1] | + |
| 203 | 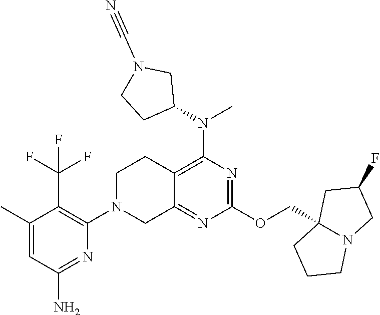 [SMILES: Cc1cc(N)nc(N2CCc3c(nc(OC[C@@]45CCCN4C[C@H](F)C5)nc3N(C)[C@@H]3CCN(C#N)C3)C2)c1C(F)(F)F] | + |
| 204 | 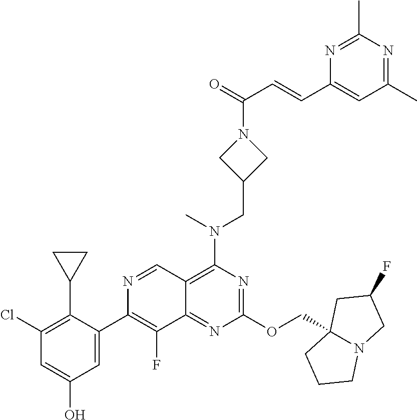 [SMILES: Cc1cc(/C=C/C(=O)N2CC(CN(C)c3nc(OC[C@@]45CCCN4C[C@H](F)C5)nc4c(F)c(-c5cc(O)cc(Cl)c5C5CC5)ncc34)C2)nc(C)n1] | ++ |
| 205 | 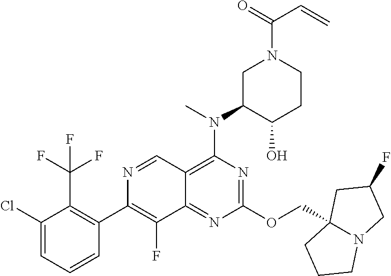 [SMILES: C=CC(=O)N1CC[C@H](O)[C@@H](N(C)c2nc(OC[C@@]34CCCN3C[C@H](F)C4)nc3c(F)c(-c4cccc(Cl)c4C(F)(F)F)ncc23)C1] | + |
| 206 | 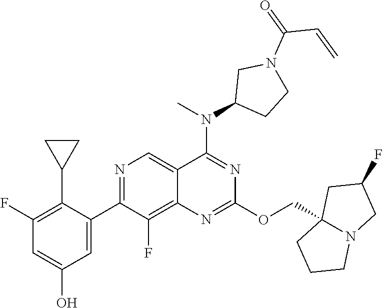 [SMILES: C=CC(=O)N1CC[C@@H](N(C)c2nc(OC[C@@]34CCCN3C[C@H](F)C4)nc3c(F)c(-c4cc(O)cc(F)c4C4CC4)ncc23)C1] | ++++ |
| 207 | 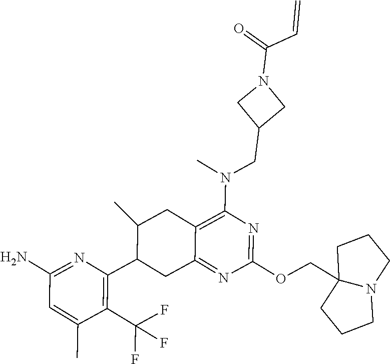 [SMILES: C=CC(=O)N1CC(CN(C)c2nc(OCC34CCCN3CCC4)nc3c2CC(C)C(c2nc(N)cc(C)c2C(F)(F)F)C3)C1] | + |
| 208 | 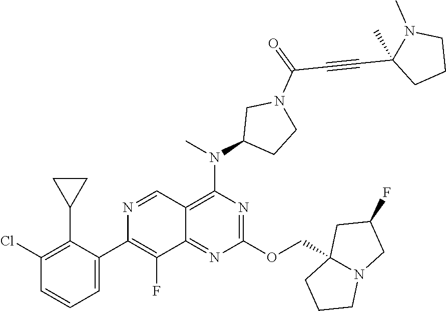 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@]2(C)CCCN2C)C1] | +++ |
| 209 | 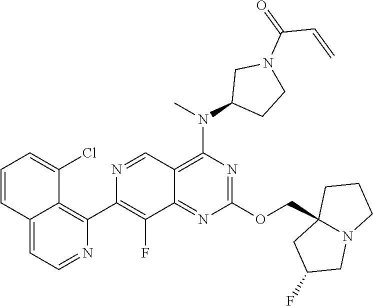 [SMILES: C=CC(=O)N1CC[C@@H](N(C)c2nc(OC[C@@]34CCCN3C[C@H](F)C4)nc3c(F)c(-c4nccc5cccc(Cl)c45)ncc23)C1] | + |
| 210 | 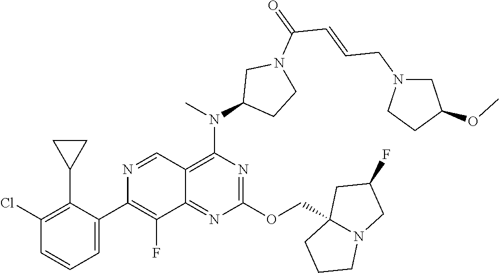 [SMILES: CO[C@H]1CCN(C/C=C/C(=O)N2CC[C@@H](N(C)c3nc(OC[C@@]45CCCN4C[C@H](F)C5)nc4c(F)c(-c5cccc(Cl)c5C5CC5)ncc34)C2)C1] | +++ |
| 211 | 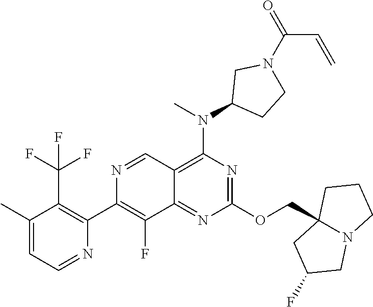 [SMILES: C=CC(=O)N1CC[C@@H](N(C)c2nc(OC[C@@]34CCCN3C[C@H](F)C4)nc3c(F)c(-c4nccc(C)c4C(F)(F)F)ncc23)C1] | + |
| 212 | 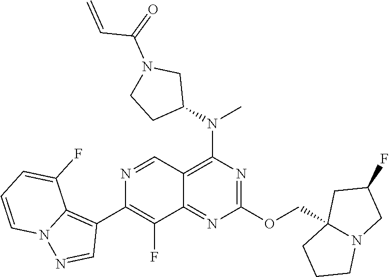 [SMILES: C=CC(=O)N1CC[C@@H](N(C)c2nc(OC[C@@]34CCCN3C[C@H](F)C4)nc3c(F)c(-c4cnn5cccc(F)c45)ncc23)C1] | + |
| 213 | 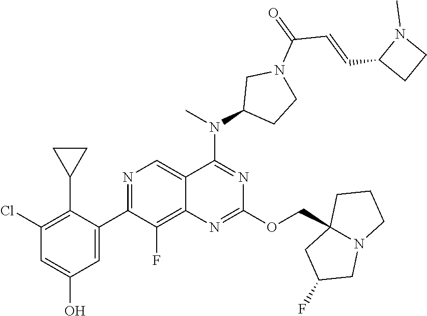 [SMILES: CN1CC[C@@H]1/C=C/C(=O)N1CC[C@@H](N(C)c2nc(OC[C@@]34CCCN3C[C@H](F)C4)nc3c(F)c(-c4cc(O)cc(Cl)c4C4CC4)ncc23)C1] | ++++ |
| 214 | 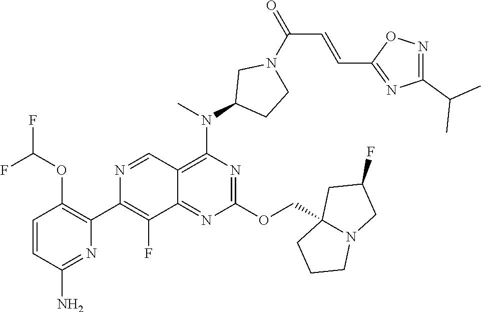 [SMILES: CC(C)c1noc(/C=C/C(=O)N2CC[C@@H](N(C)c3nc(OC[C@@]45CCCN4C[C@H](F)C5)nc4c(F)c(-c5nc(N)ccc5OC(F)F)ncc34)C2)n1] | +++ |
| 215 | 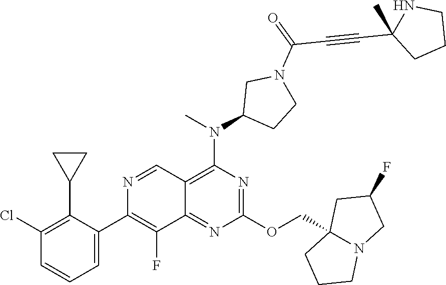 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2)C1] | ++++ |
| 216 | 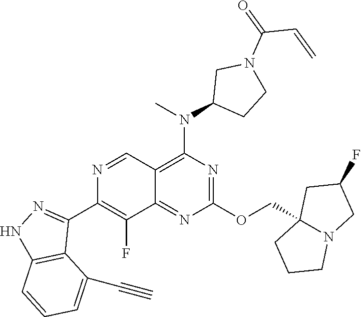 [SMILES: C#Cc1cccc2[nH]nc(-c3ncc4c(N(C)[C@@H]5CCN(C(=O)C=C)C5)nc(OC[C@@]56CCCN5C[C@H](F)C6)nc4c3F)c12] | + |
| 217 | 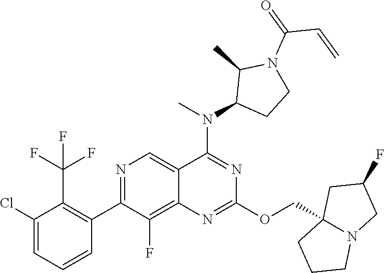 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C] | +++ |
| 218 | 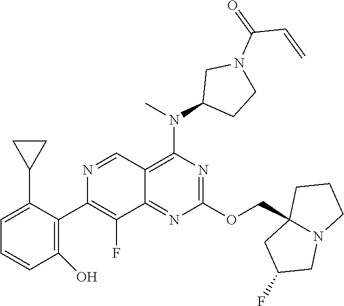 [SMILES: C=CC(=O)N1CC[C@@H](N(C)c2nc(OC[C@@]34CCCN3C[C@H](F)C4)nc3c(F)c(-c4c(O)cccc4C4CC4)ncc23)C1] | + |
| 219 | 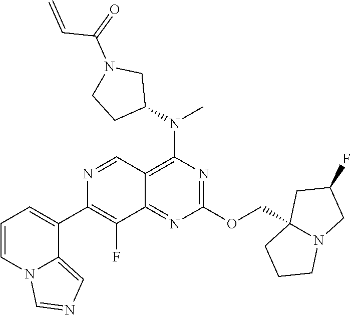 [SMILES: C=CC(=O)N1CC[C@@H](N(C)c2nc(OC[C@@]34CCCN3C[C@H](F)C4)nc3c(F)c(-c4cccn5cncc45)ncc23)C1] | + |
| 220 | 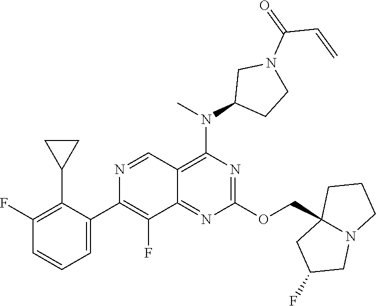 [SMILES: C=CC(=O)N1CC[C@@H](N(C)c2nc(OC[C@@]34CCCN3C[C@H](F)C4)nc3c(F)c(-c4cccc(F)c4C4CC4)ncc23)C1] | + |
| 221 | 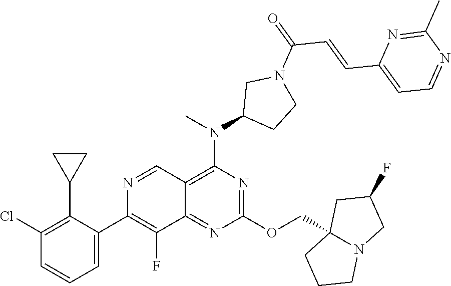 [SMILES: Cc1nccc(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)n1] | ++ |
| 222 | 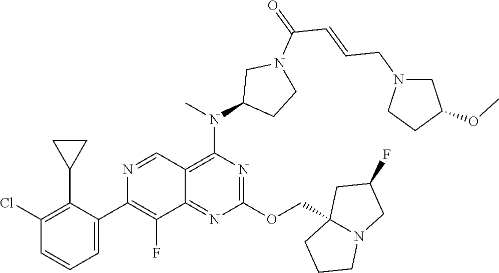 [SMILES: CO[C@@H]1CCN(C/C=C/C(=O)N2CC[C@@H](N(C)c3nc(OC[C@@]45CCCN4C[C@H](F)C5)nc4c(F)c(-c5cccc(Cl)c5C5CC5)ncc34)C2)C1] | ++ |
| 223 | 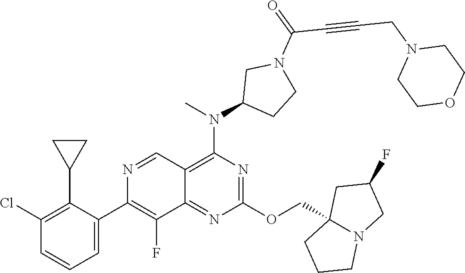 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#CCN2CCOCC2)C1] | ++ |
| 224 | 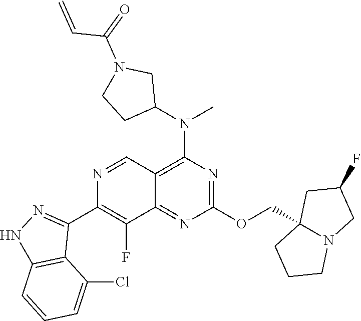 [SMILES: C=CC(=O)N1CCC(N(C)c2nc(OC[C@@]34CCCN3C[C@H](F)C4)nc3c(F)c(-c4n[nH]c5cccc(Cl)c45)ncc23)C1] | + |
| 225 | 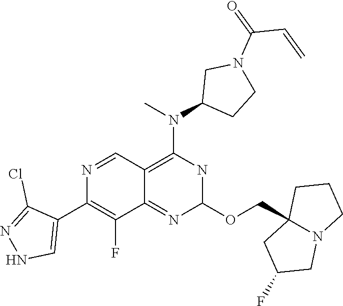 [SMILES: C=CC(=O)N1CC[C@@H](N(C)C2=c3cnc(-c4c[nH]nc4Cl)c(F)c3=NC(OC[C@@]34CCCN3C[C@H](F)C4)N2)C1] | + |
| 226 | 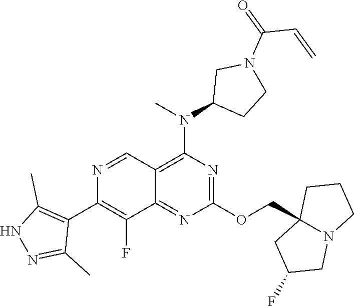 [SMILES: C=CC(=O)N1CC[C@@H](N(C)c2nc(OC[C@@]34CCCN3C[C@H](F)C4)nc3c(F)c(-c4c(C)n[nH]c4C)ncc23)C1] | |
| 227 | 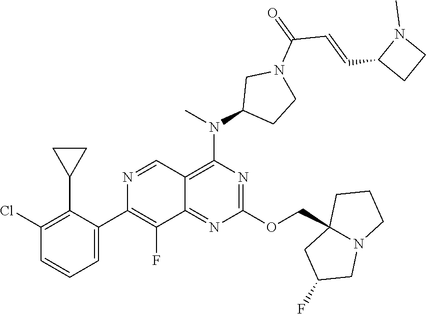 [SMILES: CN1CC[C@@H]1/C=C/C(=O)N1CC[C@@H](N(C)c2nc(OC[C@@]34CCCN3C[C@H](F)C4)nc3c(F)c(-c4cccc(Cl)c4C4CC4)ncc23)C1] | ++++ |
| 228 | 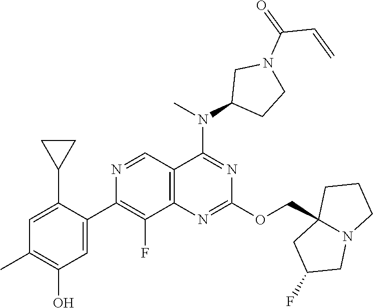 [SMILES: C=CC(=O)N1CC[C@@H](N(C)c2nc(OC[C@@]34CCCN3C[C@H](F)C4)nc3c(F)c(-c4cc(O)c(C)cc4C4CC4)ncc23)C1] | + |
| 229 | 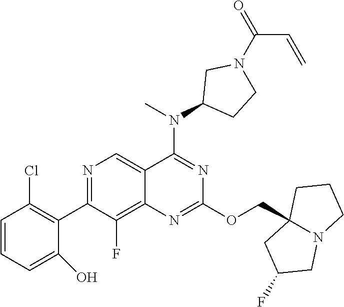 [SMILES: C=CC(=O)N1CC[C@@H](N(C)c2nc(OC[C@@]34CCCN3C[C@H](F)C4)nc3c(F)c(-c4c(O)cccc4Cl)ncc23)C1] | + |
| 230 | 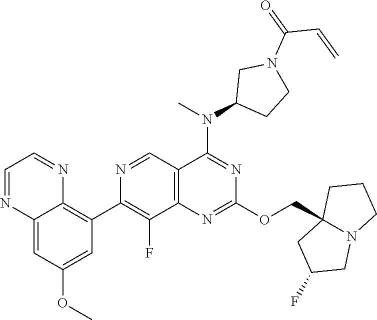 [SMILES: C=CC(=O)N1CC[C@@H](N(C)c2nc(OC[C@@]34CCCN3C[C@H](F)C4)nc3c(F)c(-c4cc(OC)cc5nccnc45)ncc23)C1] | + |
| 231 | 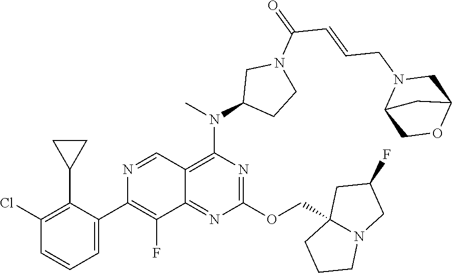 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@@H]3C[C@H]2CO3)C1] | +++ |
| 232 | 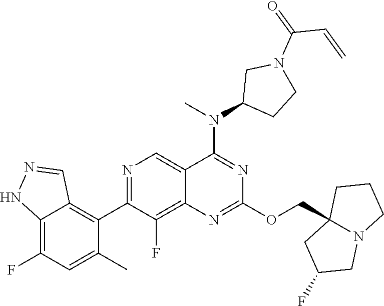 [SMILES: C=CC(=O)N1CC[C@@H](N(C)c2nc(OC[C@@]34CCCN3C[C@H](F)C4)nc3c(F)c(-c4c(C)cc(F)c5[nH]ncc45)ncc23)C1] | + |
| 233 | 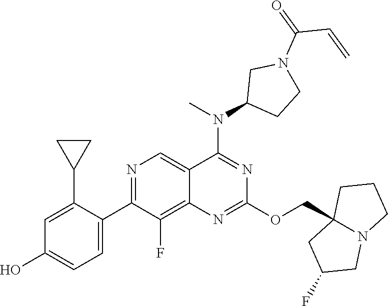 [SMILES: C=CC(=O)N1CC[C@@H](N(C)c2nc(OC[C@@]34CCCN3C[C@H](F)C4)nc3c(F)c(-c4ccc(O)cc4C4CC4)ncc23)C1] | + |
| 234 | 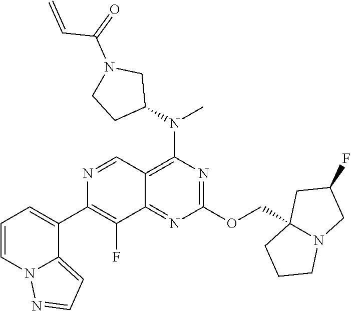 [SMILES: C=CC(=O)N1CC[C@@H](N(C)c2nc(OC[C@@]34CCCN3C[C@H](F)C4)nc3c(F)c(-c4cccn5nccc45)ncc23)C1] | + |
| 235 | 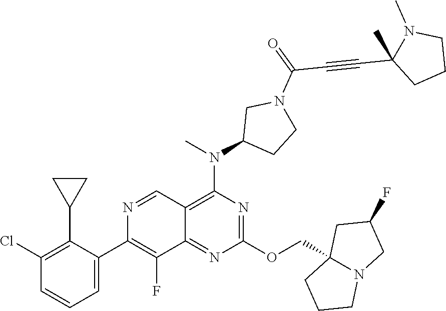 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2C)C1] | +++ |
| 236 | 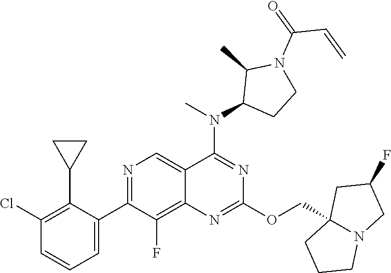 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C] | ++ |
| 237 | 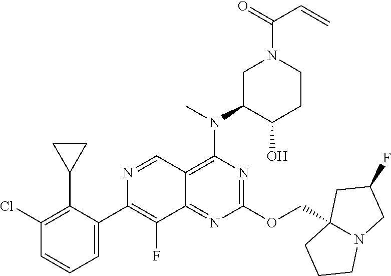 [SMILES: C=CC(=O)N1CC[C@H](O)[C@@H](N(C)c2nc(OC[C@@]34CCCN3C[C@H](F)C4)nc3c(F)c(-c4cccc(Cl)c4C4CC4)ncc23)C1] | + |
| 238 | 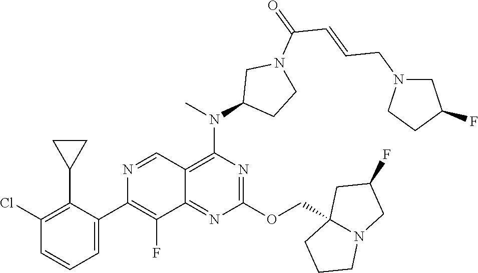 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@H](F)C2)C1] | +++ |
| 239 | 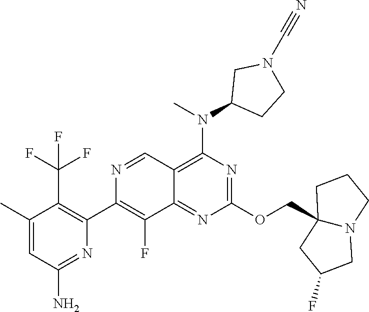 [SMILES: Cc1cc(N)nc(-c2ncc3c(N(C)[C@@H]4CCN(C#N)C4)nc(OC[C@@]45CCCN4C[C@H](F)C5)nc3c2F)c1C(F)(F)F] | ++++ |
| 240 | 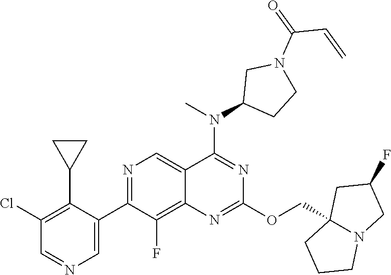 [SMILES: C=CC(=O)N1CC[C@@H](N(C)c2nc(OC[C@@]34CCCN3C[C@H](F)C4)nc3c(F)c(-c4cncc(Cl)c4C4CC4)ncc23)C1] | + |
| 241 | 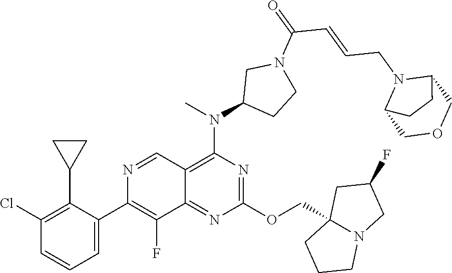 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2[C@@H]3CC[C@H]2COC3)C1] | +++ |
| 242 | 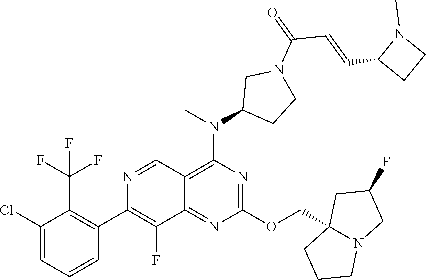 [SMILES: CN1CC[C@@H]1/C=C/C(=O)N1CC[C@@H](N(C)c2nc(OC[C@@]34CCCN3C[C@H](F)C4)nc3c(F)c(-c4cccc(Cl)c4C(F)(F)F)ncc23)C1] | +++ |
| 243 | 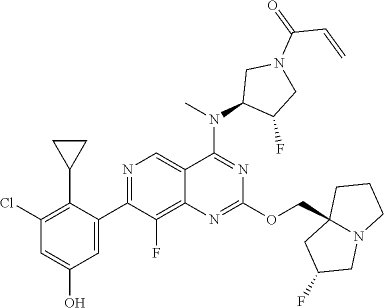 [SMILES: C=CC(=O)N1C[C@H](F)[C@@H](N(C)c2nc(OC[C@@]34CCCN3C[C@H](F)C4)nc3c(F)c(-c4cc(O)cc(Cl)c4C4CC4)ncc23)C1] | ++++ |
| 244 | 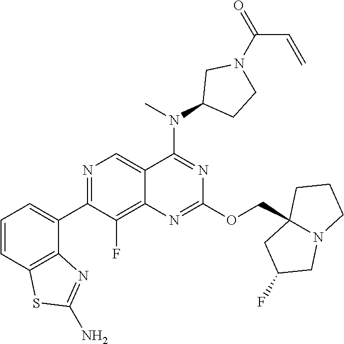 [SMILES: C=CC(=O)N1CC[C@@H](N(C)c2nc(OC[C@@]34CCCN3C[C@H](F)C4)nc3c(F)c(-c4cccc5sc(N)nc45)ncc23)C1] | +++ |
| 245 | 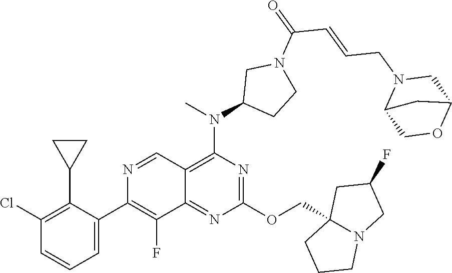 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@H]3C[C@@H]2CO3)C1] | +++ |
| 246 | 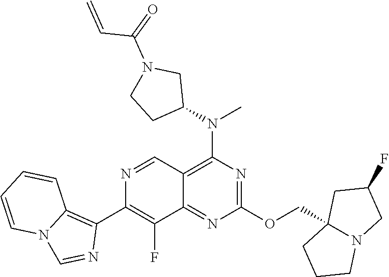 [SMILES: C=CC(=O)N1CC[C@@H](N(C)c2nc(OC[C@@]34CCCN3C[C@H](F)C4)nc3c(F)c(-c4ncn5ccccc45)ncc23)C1] | + |
| 247 | 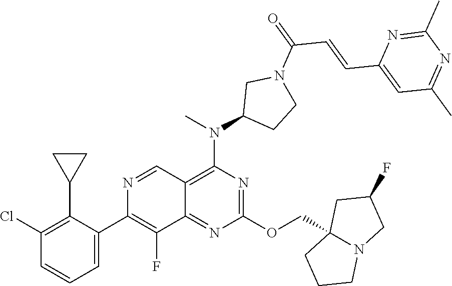 [SMILES: Cc1cc(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)nc(C)n1] | ++ |
| 248 | 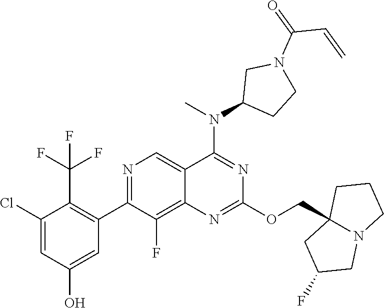 [SMILES: C=CC(=O)N1CC[C@@H](N(C)c2nc(OC[C@@]34CCCN3C[C@H](F)C4)nc3c(F)c(-c4cc(O)cc(Cl)c4C(F)(F)F)ncc23)C1] | ++++ |
| 249 | 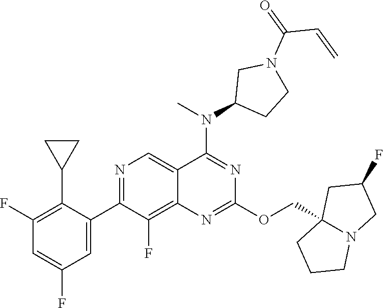 [SMILES: C=CC(=O)N1CC[C@@H](N(C)c2nc(OC[C@@]34CCCN3C[C@H](F)C4)nc3c(F)c(-c4cc(F)cc(F)c4C4CC4)ncc23)C1] | + |
| 250 | 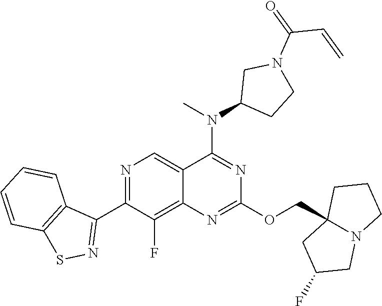 [SMILES: C=CC(=O)N1CC[C@@H](N(C)c2nc(OC[C@@]34CCCN3C[C@H](F)C4)nc3c(F)c(-c4nsc5ccccc45)ncc23)C1] | |
| 251 | 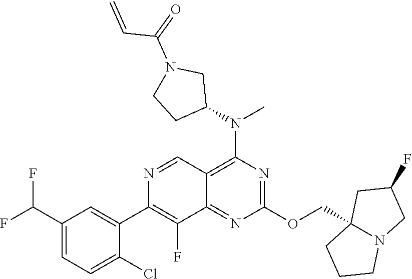 [SMILES: C=CC(=O)N1CC[C@@H](N(C)c2nc(OC[C@@]34CCCN3C[C@H](F)C4)nc3c(F)c(-c4cc(C(F)F)ccc4Cl)ncc23)C1] | + |
| 252 | 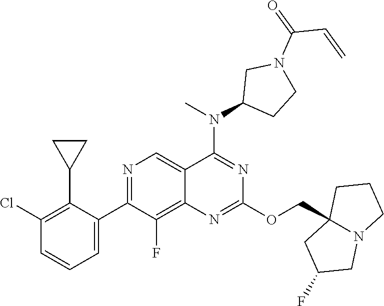 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)C1] | +++ |
| 253 | 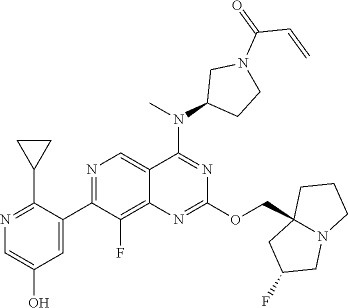 [SMILES: C=CC(=O)N1CC[C@@H](N(C)c2nc(OC[C@@]34CCCN3C[C@H](F)C4)nc3c(F)c(-c4cc(O)cnc4C4CC4)ncc23)C1] | + |
| 254 | 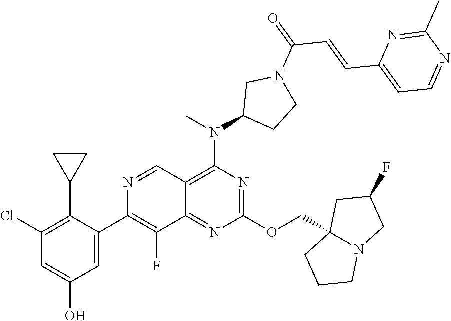 [SMILES: Cc1nccc(/C=C/C(=O)N2CC[C@@H](N(C)c3nc(OC[C@@]45CCCN4C[C@H](F)C5)nc4c(F)c(-c5cc(O)cc(Cl)c5C5CC5)ncc34)C2)n1] | ++++ |
| 255 | 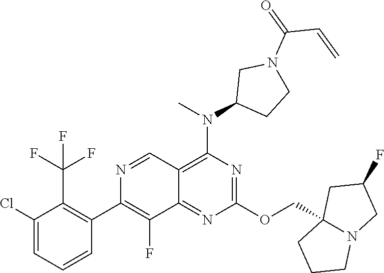 [SMILES: C=CC(=O)N1CC[C@@H](N(C)c2nc(OC[C@@]34CCCN3C[C@H](F)C4)nc3c(F)c(-c4cccc(Cl)c4C(F)(F)F)ncc23)C1] | ++ |
| 256 | 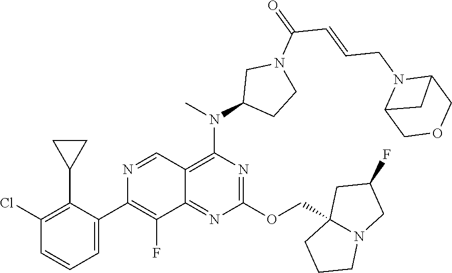 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C3COCC2C3)C1] | +++ |
| 257 | 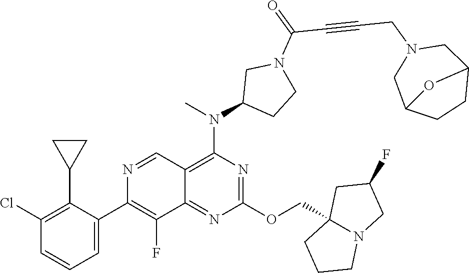 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#CCN2CC3CCC(C2)O3)C1] | +++ |
| 258 | 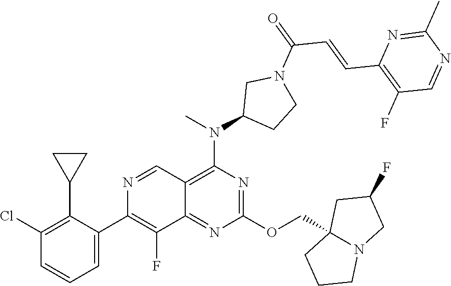 [SMILES: Cc1ncc(F)c(/C=C/C(=O)N2CC[C@@H](N(C)c3nc(OC[C@@]45CCCN4C[C@H](F)C5)nc4c(F)c(-c5cccc(Cl)c5C5CC5)ncc34)C2)n1] | ++ |
| 259 | 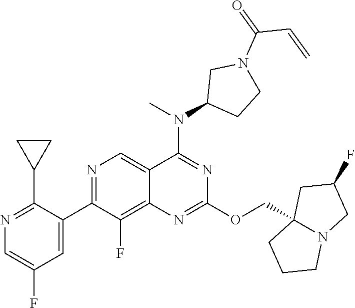 [SMILES: C=CC(=O)N1CC[C@@H](N(C)c2nc(OC[C@@]34CCCN3C[C@H](F)C4)nc3c(F)c(-c4cc(F)cnc4C4CC4)ncc23)C1] | + |
| 260 | 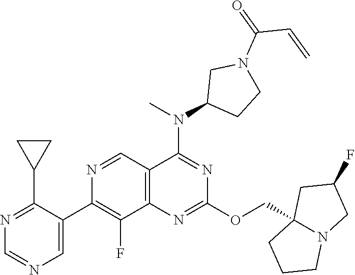 [SMILES: C=CC(=O)N1CC[C@@H](N(C)c2nc(OC[C@@]34CCCN3C[C@H](F)C4)nc3c(F)c(-c4cncnc4C4CC4)ncc23)C1] | + |
| 261 | 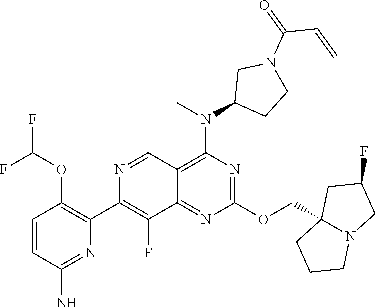 [SMILES: C=CC(=O)N1CC[C@@H](N(C)c2nc(OC[C@@]34CCCN3C[C@H](F)C4)nc3c(F)c(-c4nc(N)ccc4OC(F)F)ncc23)C1] | + |
| 262 | 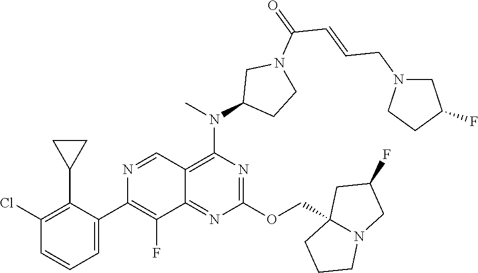 [SMILES: CN(c1nc(OC[C@@]23CCCN2C[C@H](F)C3)nc2c(F)c(-c3cccc(Cl)c3C3CC3)ncc12)[C@@H]1CCN(C(=O)/C=C/CN2CC[C@@H](F)C2)C1] | +++ |
| 263 | 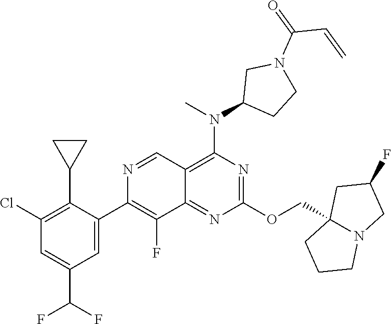 [SMILES: C=CC(=O)N1CC[C@@H](N(C)c2nc(OC[C@@]34CCCN3C[C@H](F)C4)nc3c(F)c(-c4cc(C(F)F)cc(Cl)c4C4CC4)ncc23)C1] | + |
| 264 | 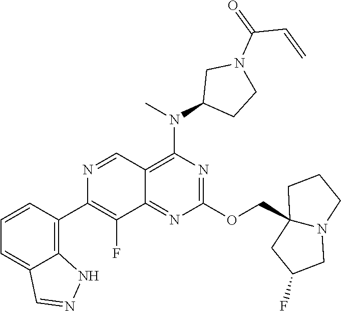 [SMILES: C=CC(=O)N1CC[C@@H](N(C)c2nc(OC[C@@]34CCCN3C[C@H](F)C4)nc3c(F)c(-c4cccc5cn[nH]c45)ncc23)C1] | + |

IC50 ++++ is less than 10 nM, +++ is 10 to less than 100 nM, ++ is 100 to less than 500 nM, + is greater or equal to 500 nM; if the assay data column entry is blank, the compounds are inactive.

### Example 180: Inhibition of KRAS^{G12C} and PI3Ka Binding

The AlphaScreen technology is used to determine IC₅₀S for compound inhibition of KRAS G12C (present as the Cys-light (C51S, C80L and C118S), truncated version comprising amino acids 1-169) and PI3Ka interaction. Compounds are diluted in 10000 DMSO and each compound concentration is spotted at 200 nl/well onto low volume, white 384 well plates. The KRAS G12C contains a biotin-AviTag and the PI3Ka, as Ras-binding domain (amino acids 157-300, RBD), is His-tagged. KRAS G12C is preloaded with the GTP analogue Guanosine 5′-[β,γ-imido]triphosphate (GMPPNP). The KRAS G12C is diluted in 25 mM Hepes, pH 7.4, 150 mM NaCl, 5 mM MgCl₂, 0.01% TritonX-100 and 10 μM GMPPNP and added at 10 ul/well to compound-spotted plates resulting in a DMSO concentration of 2%. Plates are incubated for 2 hours. A mixture of RBD and the AlphaScreen streptavidin donor and nickel chelate acceptor beads diluted in 25 mM Hepes, pH 7.4, 150 mM NaCl, 5 mM MgCl₂, 0.01% TritonX-100 and 2% DMSO are then added at 10 ul/well and incubated for 60-90 minutes before the samples are read for emission at 570 nm after excitation of the donor beads at 680 nm. All incubations are performed at room temperature. The final top compound concentration is 50 μM with 1:3 titrations for 10-point dose response curves. Final assay conditions are 1.5 nM KRAS G12C, 100 nM RBD, 1.25 ug/ml of AlphaScreen donor beads and 10 ug/ml AlphaLISA acceptor beads. IC₅₀s are determined using nonlinear regression fit of [inhibitor] vs. response (4 parameters).

A counter assay is also set up to rule out inhibitors of the AlphaScreen technology itself. Compound plates are incubated for 2 hours as above with buffer only. The AlphaScreen beads are added as above except an unrelated biotinylated His-tagged peptide is substituted for the RBD. Samples are read and analyzed as above.

### Example 181: MCF10A (G12C or G12C-A59G)-KRAS Cell Viability Assay

MCF10A (ATCC, cat. CRL-10317) cells are maintained in MEBM (Lonza, cat. CC-3151) with 1% horse serum (Sigma, cat. H1270), MEGM mammary epithelial cell growth medium SingleQuotsKit (Lonza, cat. CC-4146) and 25 ng/ml Cholera toxin (Sigma, cat. C8052). These cells are transduced with either KRAS G12C or G12C/A59G followed by puromycin selection to generate stably expressing cells. For the cell viability assay, 1000 cells of either MCF10A KRAS G12C or MCF10A G12C/A59G are plated in 384-well spheroid microplate (Corning, cat. 3830). The following day, cells are treated with compounds (10 uM top concentration, 3-fold dilution, and 11 doses). 10 uM Tremetinib (MCE, cat. HY-10999/CS-0060) is used as control. The Tecan: HP D300E is used to dispense the compounds. After five days of incubation, celltiter-glo luminescent assay kit (Promega, cat. G7573) is used according to manufacturer's protocol to measure cellular viability using a BioTek plate reader. The data is then imported to and processed in Dotmatics where EC50s were calculated using the Lavenberg-Marquardt 4 parameters fitting procedure, with difference gradients.

### Example 182: Treatment of Human Patients

A human patient suffering from a cancer, (e.g., a KRAS mediated cancer, as disclosed herein) can be administered a therapeutically effective dose of a compound disclosed herein (e.g., a compound of Table 1). The treatment can slow down or halt the growth of a tumor, reduce a tumor volume or mass, or eradicate the tumor in the patient.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed:

1. A compound of Formula A, Formula B, or Formula C: 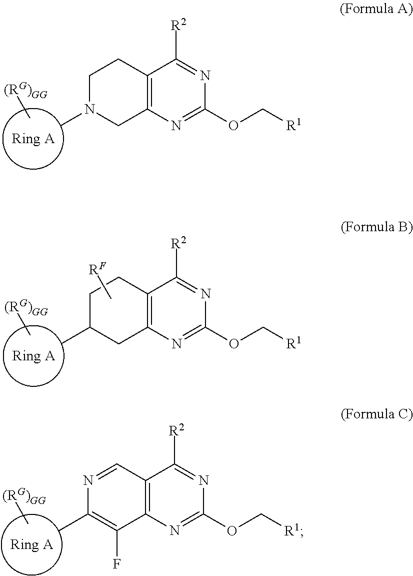
or a salt thereof, and/or an isotopologue thereof, wherein:
Ring A is a 6-membered aryl or a 5-10 membered heteroaryl;
R^{F} is selected from the group consisting of H, halo, C₁-C₄ alkyl, C₁-C₄ alkoxy, C₁-C₄ haloalkyl and C₁-C₄ haloalkoxy;
each R^{G} is independently selected from halo, —OH, —NH₂, C₁-C₄ alkyl, C₁-C₄ alkoxy, C₁-C₄ haloalkyl, C₁-C₄ haloalkoxy, C₃-C₆ cycloalkyl and C₂-C₃ alkynyl;
each GG is independently 0, 1, 2 or 3;
R¹ is a 4-8 membered saturated carbocyclic or heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy;
R² is selected from the group consisting of R^{2b}, R^{2c} and R^{2e};
R^{2b} is —NR¹⁰R¹¹;
R¹⁰ is selected from the group consisting of H, C₁-C₄ alkyl, C₁-C₄ alkoxy, C₁-C₄ haloalkyl, and C₁-C₄ haloalkoxy; and
R¹¹ is —(CH₂)_{w}—R¹³; or
R¹⁰ and R¹¹ together with the nitrogen to which they are attached form a 4-8 membered saturated heterocyclic group comprising a second nitrogen as the sole additional heteroatom within the ring atoms, wherein the second nitrogen of the 4-8 membered saturated heterocyclic group is substituted with cyano, and the 4-8 membered saturated heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
R¹³ is a 4-5 membered saturated heterocyclic group comprising one nitrogen as the sole heteroatom within the ring atoms, wherein the nitrogen is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo; or
R¹³ is a 6 membered saturated heterocyclic group comprising one or two nitrogens as the sole heteroatom(s) within the ring atoms, wherein one of the nitrogens is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom; or
R¹³ is a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) is substituted with cyano, and wherein the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo, provided that the optional hydroxy, CN, cyanoalkyl and halo substituents are not attached to a heteroatom;
w is 0, 1, or 2;
R^{2c} is —NR¹⁵R¹⁶;
R¹⁵ is H, C₁-C₄ alkyl, C₁-C₄ alkoxy, C₁-C₄ haloalkyl, or C₁-C₄ haloalkoxy; and
R¹⁶ is —(CH₂)_{y}—R²¹;
R²¹ is selected from:
a 4-5 membered saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom; or
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo, provided that the optional hydroxy, CN, cyanoalkyl and halo substituents are not attached to a heteroatom;
R¹⁸ is selected from the group consisting of hydrogen, —COOH, —C(O)O—C₁-C₄ alkyl, —C(O)O—C₁-C₄ haloalkyl, —C(O)—C₁-C₄ alkyl, —C(O)—C₁-C₄ haloalkyl, —C(O)NR²²R²³, —(CH₂)_{z}—NR²²R²³, —(CH₂)ᵤ—R³⁴, —(C₁-C₂ alkyl)-(C₁-C₂ alkoxy), —S(O)₂—C₁-C₄ alkyl, —S(O)₂—C₁-C₄ haloalkyl, and R³⁵;
R¹⁹ is selected from the group consisting of hydrogen, C₁-C₄ alkyl, C₁-C₄ alkoxy, C₁-C₄ haloalkyl, and C₁-C₄ haloalkoxy;
R²⁰ is selected from the group consisting of hydrogen, C₁-C₄ alkyl, C₁-C₄ alkoxy, C₁-C₄ haloalkyl, and C₁-C₄ haloalkoxy;
R²² and R²³ are independently selected from the group consisting of hydrogen, C₁-C₄ alkyl, C₁-C₄ alkoxy, C₁-C₄ haloalkyl, and C₁-C₄ haloalkoxy;
R³⁴ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl;
R³⁵ is a 5-6 membered heteroaryl group optionally substituted with 0, 1 or 2 substituents independently selected from halo, hydroxy, C₁-C₄ alkyl, C₁-C₄ hydroxyalkyl, C₁-C₄ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl;
y is 0, 1, or 2;
z is 1 or 2;
q is 0 or 1;
u is 0, 1 or 2;
R^{2e} is —NR²⁸R²⁹;
R²⁸ is H, C₁-C₄ alkyl, C₁-C₄ alkoxy, C₁-C₄ haloalkyl, or C₁-C₄ haloalkoxy; and
R²⁹ is —(CH₂)ₜ—R³⁰;
R³⁰ is selected from:
a 4-5 membered saturated heterocyclic group comprising one nitrogen as the sole heteroatom within the ring atoms, wherein the nitrogen ring atom of the heterocyclic group is substituted with —C(O)C≡CR^{3′} and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated heterocyclic group comprising one or two nitrogens as the sole heteroatom(s) within the ring atoms, wherein one of the nitrogens of the heterocyclic group is substituted with —C(O)C≡CR^{3′}, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom; and
a 7 membered saturated heterocyclic group comprising one nitrogen, and optionally one additional heteroatom selected from nitrogen, oxygen, and sulfur, as the sole heteroatom(s) within the ring atoms, wherein one of the nitrogen ring atom(s) of the heterocyclic group is substituted with —C(O)C≡CR^{3′}, and wherein the heterocyclic group is not further substituted or is further substituted with 1 substituent selected from the group consisting of hydroxy, CN, C₁-C₄ cyanoalkyl, C₁-C₄ alkyl, C₁-C₄ alkoxy, C₁-C₄ haloalkyl, C₁-C₄ haloalkoxy, or halo, provided that the optional hydroxy, CN, cyanoalkyl and halo substituents are not attached to a heteroatom;
R³¹ is selected from the group consisting of —(CH₂)ᵥ—NR³²R³³ and —(CH₂)ₚ—R³⁶;
R³² and R³³ are independently selected from the group consisting of hydrogen, C₁-C₄ alkyl, C₁-C₄ alkoxy, C₁-C₄ haloalkyl, and C₁-C₄ haloalkoxy;
t is 0, 1, or 2;
v is 1 or 2;
p is 0, 1 or 2;
R³⁶ is a 4-10 membered heterocycle which is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl;

2. The compound of claim 1, wherein the compound is a compound of Formula A, or a salt thereof.

3. The compound of claim 1, wherein the compound is a compound of Formula B, or a salt thereof.

4. The compound of claim 1, wherein the compound is a compound of Formula C, or a salt thereof.

5. The compound of claim 1 or 3, wherein R^{F} is methyl.

6. The compound of any one of claims 1-5, wherein Ring A is selected from phenyl, pyridinyl and isoquinolinyl.

7. The compound of any one of claims 1-5, wherein Ring A is selected from phenyl, pyridin-2-yl, pyridin-4-yl, and isoquinolin-1-yl.

8. The compound of any one of claims 1-5, wherein each moiety represented by 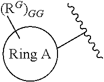
is independently selected from the group consisting of

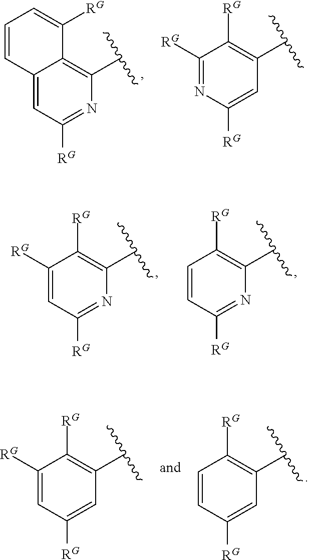

9. The compound of any one of claims 1-5, wherein each moiety represented by 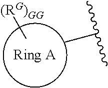
independently selected from the group consisting of

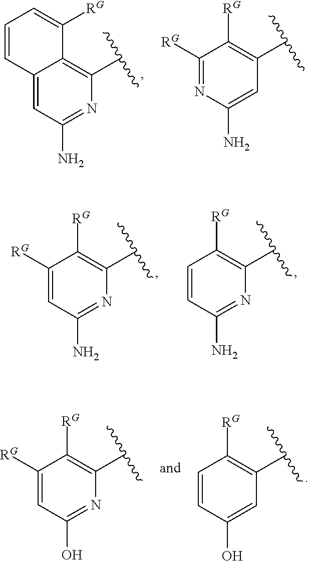

10. The compound of any one of claims 1-9, wherein each R^{G} is independently selected from —F, —Cl, -Me, —CF₃ and cyclopropyl.

11. The compound of any one of claims 1-6, wherein each moiety represented by 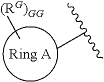
is independently selected from the group consisting of

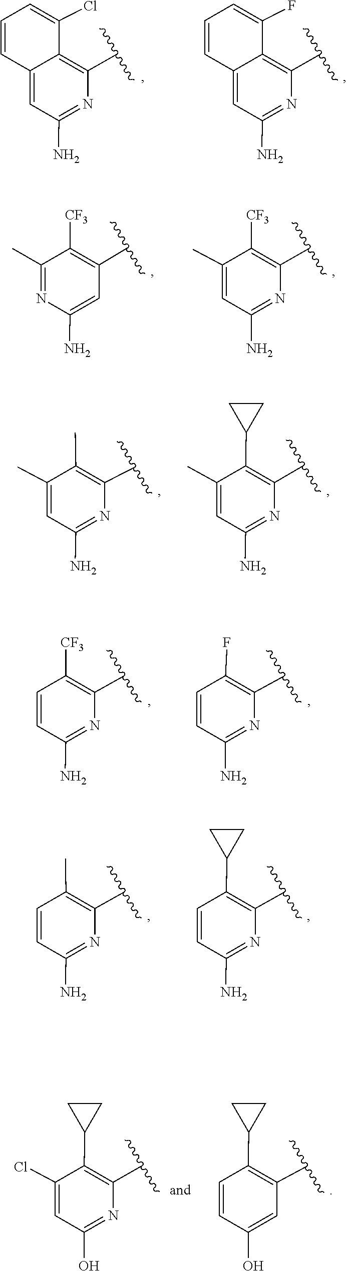

12. The compound of any one of claims 1-11, wherein R¹ is a 4-8 membered saturated monocyclic carbocyclic or monocyclic heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

13. The compound of any one of claims 1-11, wherein R¹ is a 4-8 membered saturated bicyclic carbocyclic or bicyclic heterocyclic group comprising one nitrogen as the sole heteroatom within the ring atoms, wherein the carbocyclic or heterocyclic group is substituted with 0, 1, 2 or 3 substituents independently selected from halo, hydroxy, C₁-C₄ alkyl, spiro C₃-C₄ cycloalkyl, C₁-C₄ alkoxy, C₁-C₄ haloalkyl, and C₁-C₄ haloalkoxy.

14. The compound of any one of claims 1-13, wherein the carbocyclic or heterocyclic group of R¹ is unsubstituted, or is substituted with one fluoro.

15. The compound of any one of claims 1-11, wherein R¹ is selected from the group consisting of:

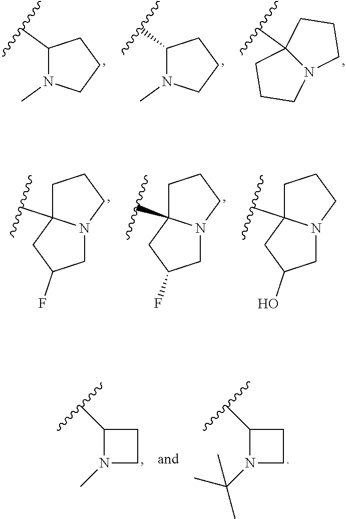

16. The compound of any one of claims 1-11, wherein R¹ is selected from the group consisting of:

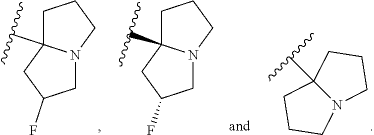

17. The compound of any one of claims 1-16, wherein R² is R^{2b}.

18. The compound of any one of claims 1-17, wherein R¹⁰ is methyl.

19. The compound of any one of claims 1-18, wherein R¹¹ is —(CH₂)_{w}—R¹³.

20. The compound of any one of claims 1-19, wherein w is 0 or 1.

21. The compound of any one of claims 1-20, wherein R¹³ is a 4-7 membered saturated heterocyclic group comprising one nitrogen as the sole heteroatom within the ring atoms, wherein the nitrogen is substituted with cyano, and wherein the heterocyclic group is optionally further substituted with 1 substituent selected from the group consisting of hydroxy, CN, C₁-C₄ cyanoalkyl, C₁-C₄ alkyl, C₁-C₄ alkoxy, or halo.

22. The compound of any one of claims 1-21, wherein the heterocyclic group of R¹³ is not further substituted.

23. The compound of any one of claims 1-21, wherein the heterocyclic group of R¹³ is further substituted with methyl, methoxy, or fluoro.

24. The compound of any one of claims 1-21, wherein R¹¹ is selected from the group consisting of:

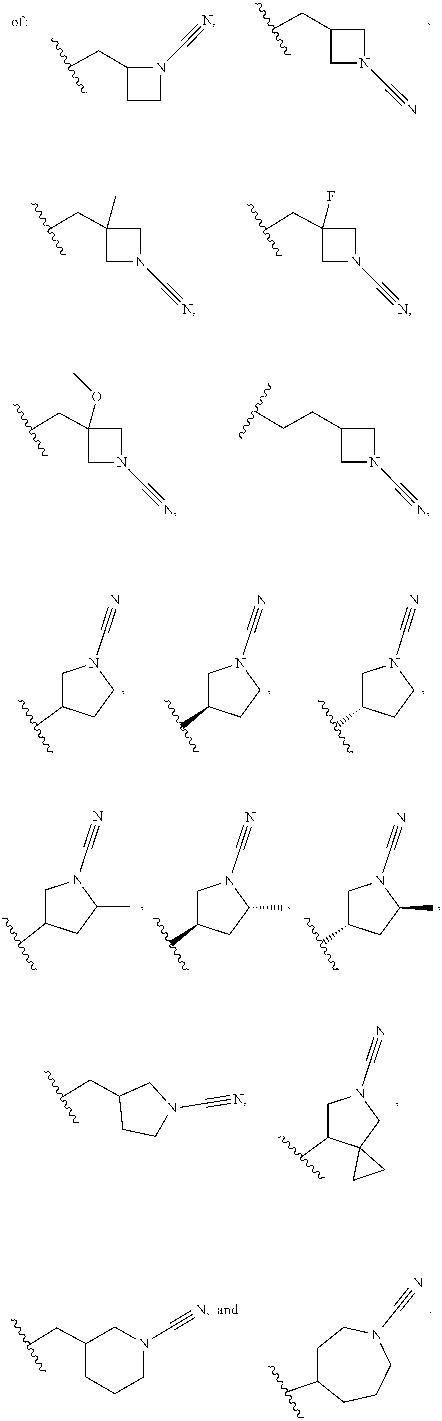

25. The compound of any one of claims 1-21, wherein R¹¹ is selected from the group consisting of:

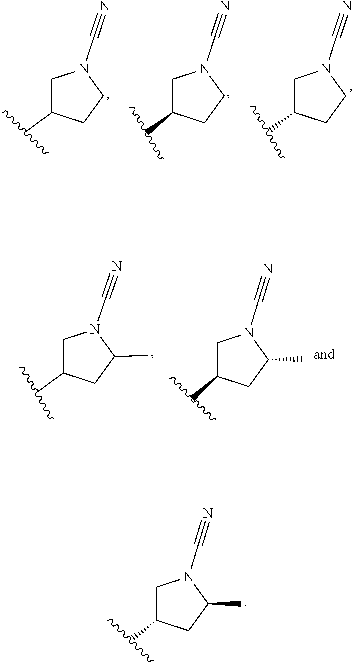

26. The compound of any one of claims 1-17, wherein R¹⁰ and R¹¹ together with the nitrogen to which they are attached form a 4-8 membered saturated heterocyclic group comprising a second nitrogen as the sole additional heteroatom within the ring atoms, wherein the second nitrogen of the 4-8 membered saturated heterocyclic group is substituted with cyano, and the 4-8 membered saturated heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

27. The compound of any one of claims 1-17, wherein the 4-8 membered saturated heterocyclic group formed by R¹⁰ and R¹¹ together with the nitrogen to which they are attached is selected from the group consisting of: 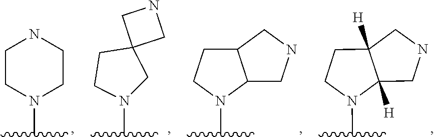
wherein the second nitrogen atom is substituted with cyano and the heterocyclic group is optionally further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

28. The compound of any one of claims 1-17, wherein the 4-8 membered saturated heterocyclic group formed by R¹⁰ and R¹¹ together with the nitrogen to which they are attached is 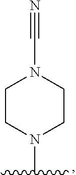 [SMILES: *N1CCN(C#N)CC1]
optionally further substituted with 1 instance of —CH₂CN.

29. The compound of any one of claims 1-16 and 18-28, wherein R² is R^{2e}.

30. The compound of any one of claims 1-16 and 18-29, wherein R¹⁵ is methyl.

31. The compound of any one of claims 1-16 and 18-30, wherein y is 0 or 1.

32. The compound of any one of claims 1-16 and 18-31 wherein R²¹ is selected from:
a 4-5 membered saturated monocyclic heterocyclic group comprising one nitrogen as the sole heteroatom within the ring atoms, wherein the nitrogen ring atom is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo;
a 6 membered saturated monocyclic heterocyclic group comprising one or two nitrogens as the sole heteroatom(s) within the ring atoms, wherein one of the nitrogens is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom; and
a 7 membered saturated monocyclic heterocyclic group comprising one nitrogen, and optionally one additional heteroatom selected from nitrogen, oxygen, and sulfur, as the sole heteroatom(s) within the ring atoms, wherein one of the nitrogen ring atom(s) is substituted with —C(O)C(R¹⁹)═C(R²⁰)R¹⁸, and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, or halo provided that the optional hydroxy, CN, cyanoalkyl and halo substituents are not attached to a heteroatom.

33. The compound of any one of claims 1-16 and 18-31, wherein R²¹ is a 4-5 membered monocyclic saturated heterocyclic group comprising one nitrogen as the sole heteroatom within the ring atoms, wherein the nitrogen ring atom of the heterocyclic group is substituted with —C(O)C(R¹⁹)═C(R²⁰)R¹⁸ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

34. The compound of any one of claims 1-16 and 18-33 wherein the heterocyclic group of R²¹ is not further substituted.

35. The compound of any one of claims 1-16 and 18-33 wherein the heterocyclic group of R²¹ is further substituted with 1 substituent selected from the group consisting of hydroxy, CN, Me, —CH₂CN and F.

36. The compound of any one of claims 1-16 and 18-31, wherein the heterocyclic group of R²¹ is selected from the group consisting of: 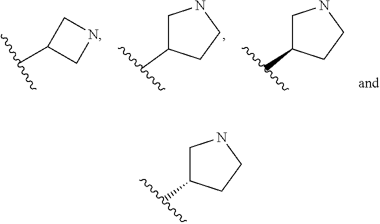
wherein the ring nitrogen of the heterocyclic group is substituted with —C(O)C(R¹⁹)═C(R²⁰)R¹⁸ and the heterocyclic group is not further substituted, or is substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

37. The compound of claim 36, wherein the heterocyclic group of R²¹ is not further substituted.

38. The compound of claim 36, wherein the heterocyclic group of R²¹ is further substituted with 1 substituent selected from the group consisting of hydroxy, CN, Me, —CH₂CN and F.

39. The compound of any one of claims 1-16 and 18-31, wherein R¹⁶ is selected from the group consisting of:

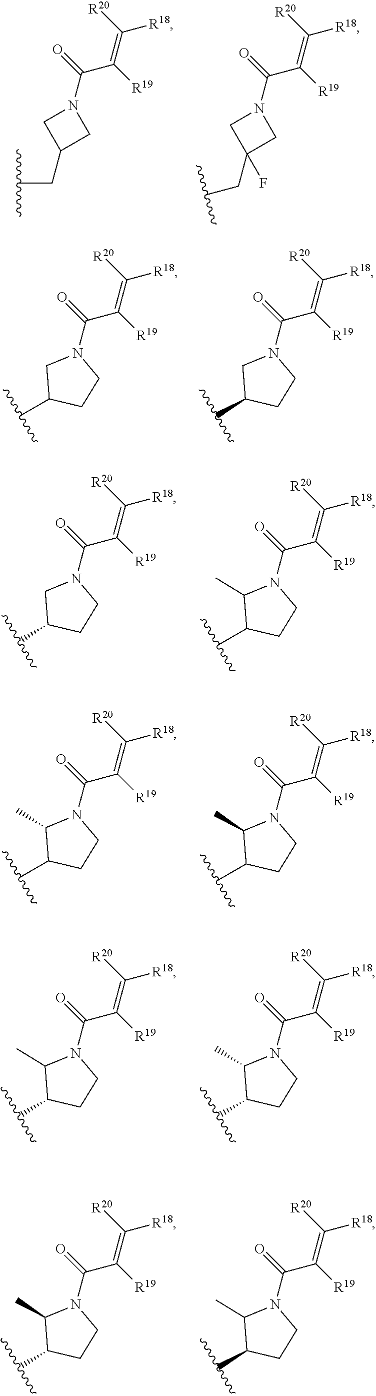

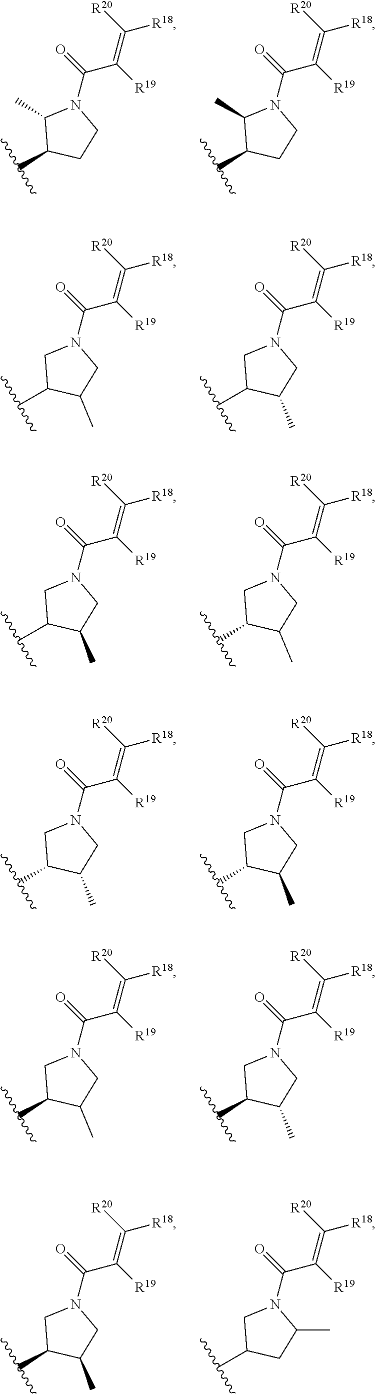

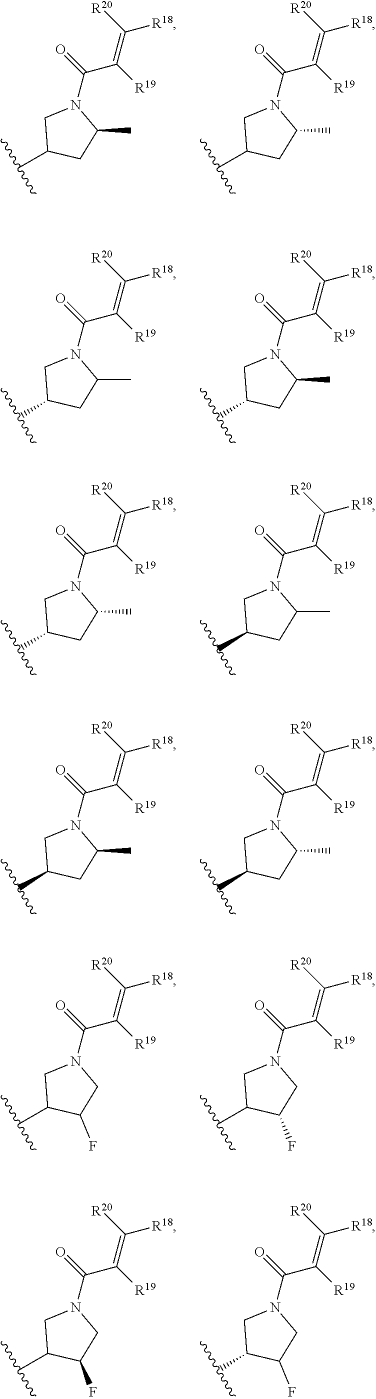

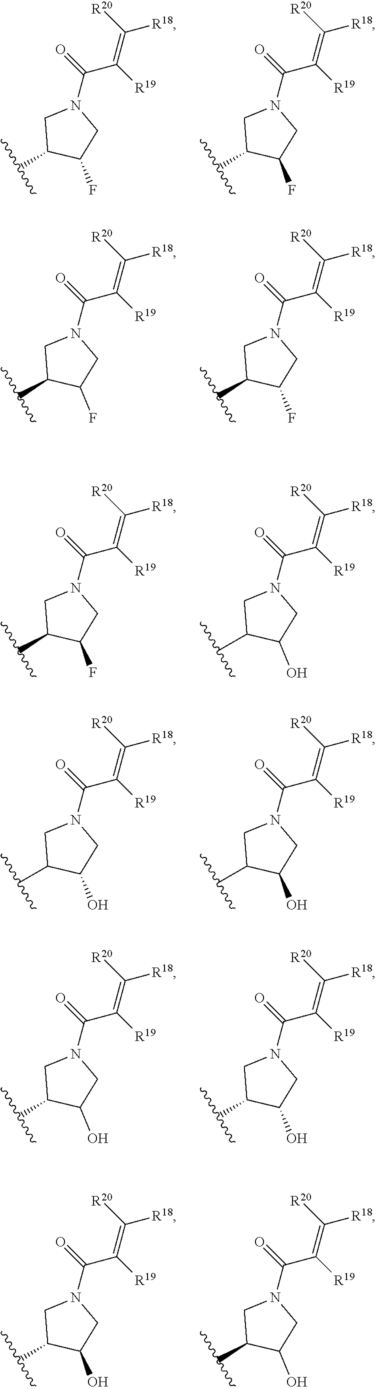

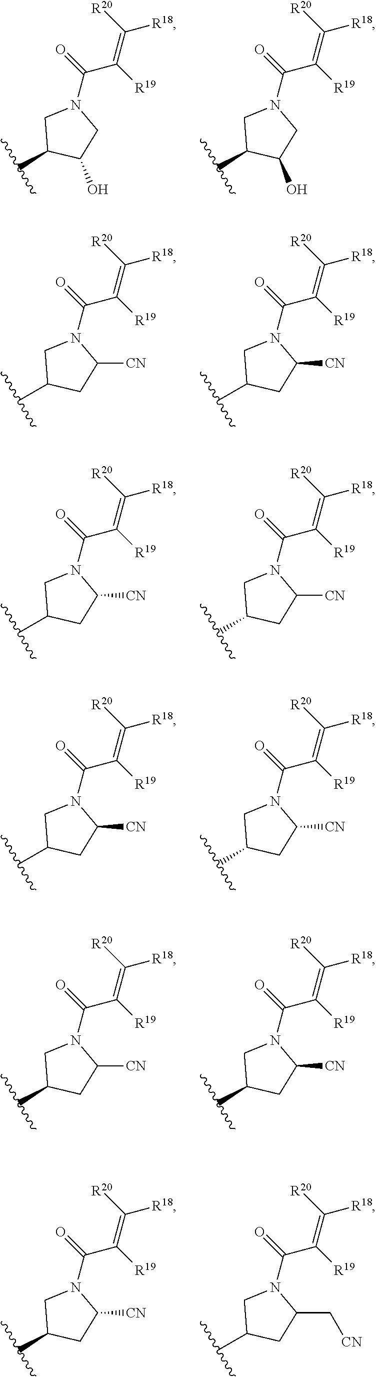

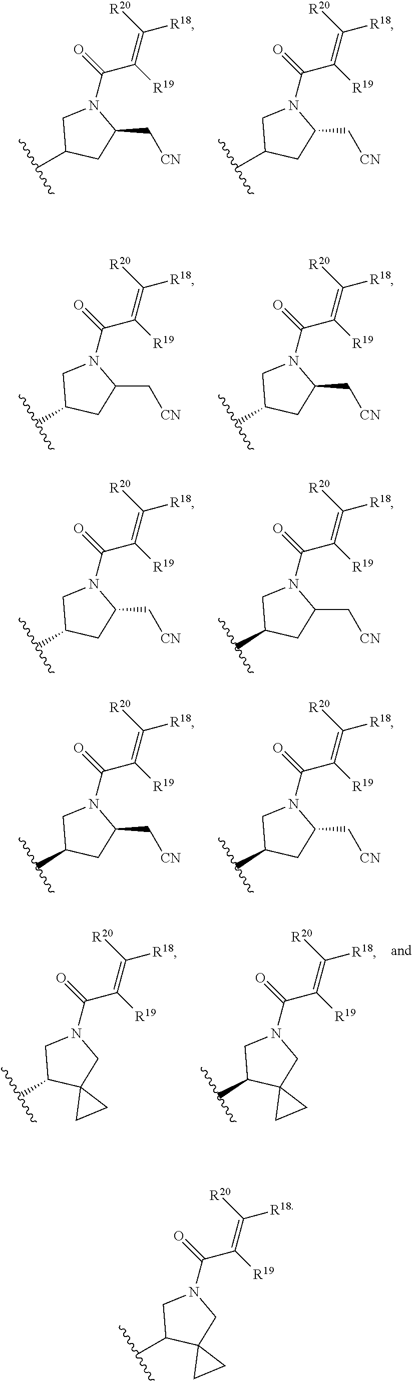

40. The compound of any one of claims 1-16 and 18-31, wherein R¹⁶ is selected from the group consisting of:

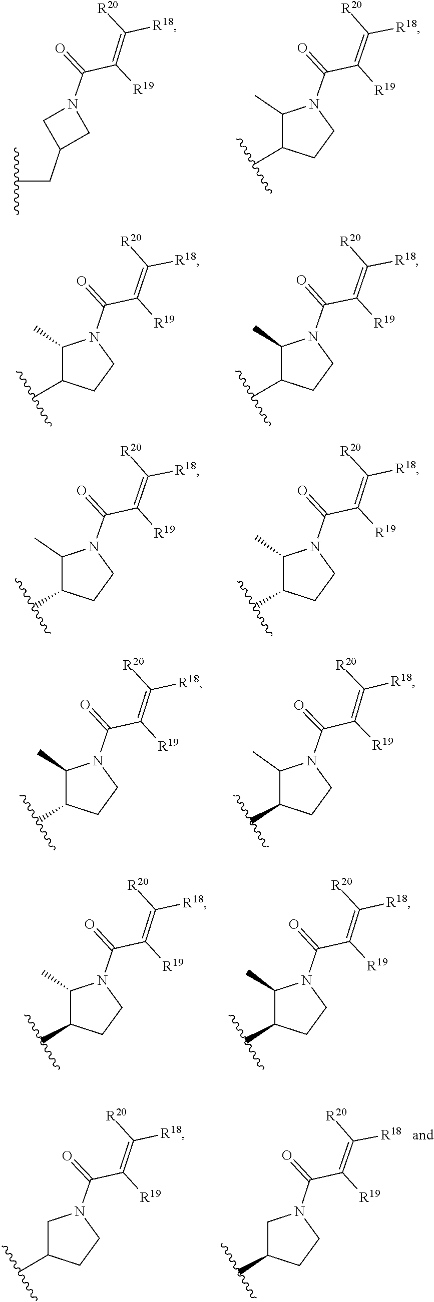

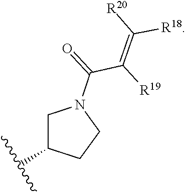

41. The compound of any one of claims 1-16 and 18-40, wherein R¹⁹ is hydrogen.

42. The compound of any one of claims 1-16 and 18-41, wherein R²⁰ is hydrogen.

43. The compound of any one of claims 1-16 and 18-42, wherein R¹⁸ is selected from the group consisting of hydrogen, —COOH, —C(O)O—C₁-C₄ alkyl, —C(O)—C₁-C₄ alkyl, —C(O)NR²²R²³, —(CH₂)_{z}—NR²²R²³, —(CH₂)ᵤ—R³⁴, —(C₁-C₂ alkyl)-(C₁-C₂ alkoxy), —S(O)₂—C₁-C₄ alkyl, and R.

44. The compound of any one of claims 1-16 and 18-43, wherein R²² and R²³ are independently selected from methyl and ethyl.

45. The compound of any one of claims 1-16 and 18-43, wherein R³⁴ is a 4-7 membered monocyclic heterocycle containing a nitrogen atom and zero, one or two additional heteroatoms selected from oxygen and sulfur, including sulfur dioxide, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

46. The compound of any one of claims 1-16 and 18-45, wherein the monocyclic heterocycle of R³⁴ is substituted with 0 or 1 instance of methyl.

47. The compound of any one of claims 1-16 and 18-45, wherein R³⁴ is selected from azetidinyl, pyrrolidinyl and morpholinyl substituted with 0 or 1 instance of methyl.

48. The compound of one of claims 1-16 and 18-45, wherein R³⁴ is azetidinyl substituted with 0 or 1 instance of methyl.

49. The compound of any one of claims 1-16 and 18-45, wherein R³⁴ is pyrrolidinyl substituted with 0 or 1 instance of methyl.

50. The compound of any one of claims 1-16 and 18-45, wherein R³⁴ is morpholinyl substituted with 0 or 1 instance of methyl.

51. The compound of any one of claims 1-16 and 18-50, wherein the attachment point for R³⁴ is a carbon atom.

52. The compound of claim 51, wherein R³⁴ is selected from the group consisting of:

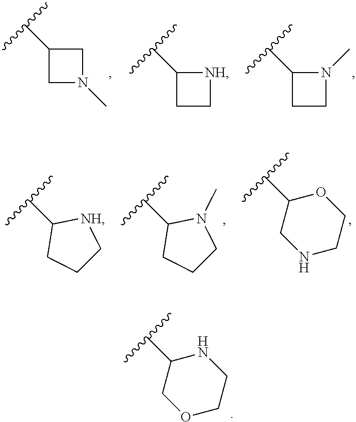

53. The compound of claim 51, wherein R³⁴ is selected from the group consisting of

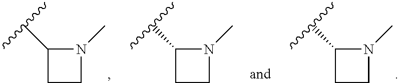

54. The compound of any one of claims 1-16 and 18-44, wherein R³⁴ is a 4-10 membered heterocycle containing a nitrogen atom and zero, one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

55. The compound of claim 54, wherein R³⁴ is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 2-oxa-5-azabicyclo[2.2.2]octane, 7-oxa-2-azabicyclo[3.3.1]nonane, 8-oxa-3-azabicyclo[3.2.1]octane, 9-oxa-3-azabicyclo[3.3.1]nonane, 6-oxa-8-azabicyclo[3.2.2]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 1,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

56. The compound of claim 54, wherein R³⁴ is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

57. The compound of any one of claims 54 to 56, wherein the attachment point for R³⁴ is the nitrogen atom of the heterocycle.

58. The compound of claim 57, wherein R³⁴ is selected from the group consisting of: 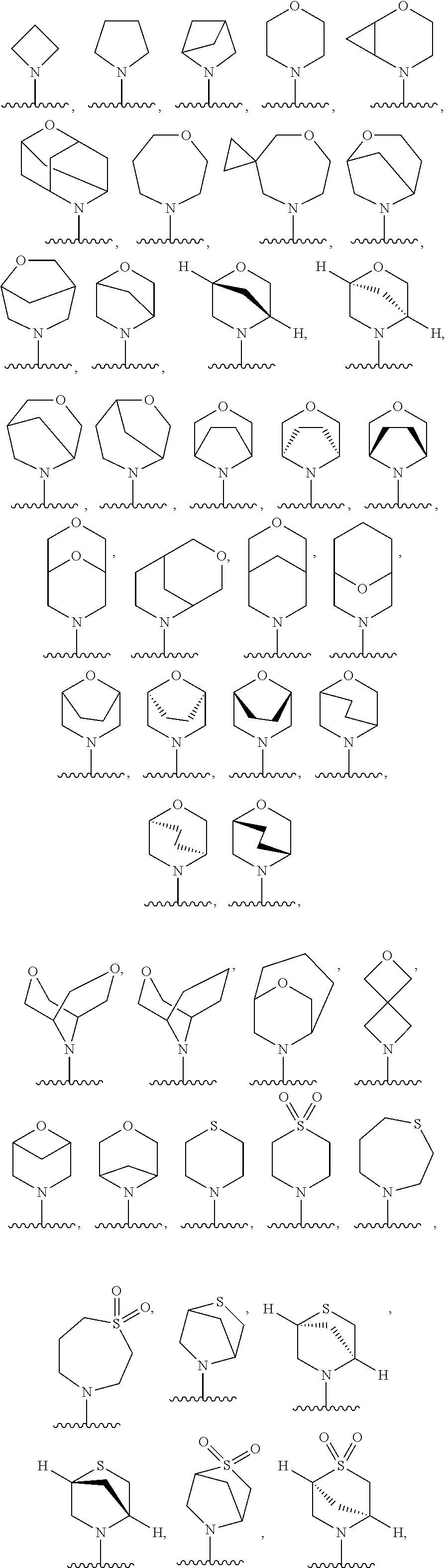 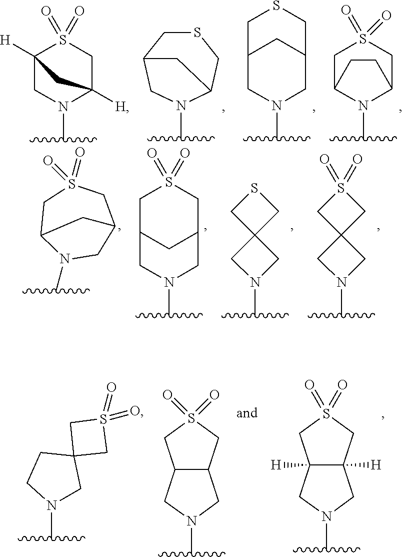
each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

59. The compound of claim 57, wherein R³⁴ is selected from the group consisting of: 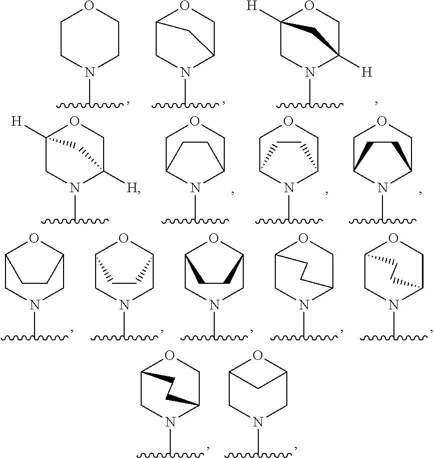
each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

60. The compound of claim 57, wherein R³⁴ is 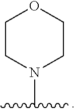 [SMILES: *N1CCOCC1]
substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

61. The compound of any one of claims 54 to 60, wherein the 4-10 membered heterocycle of R³⁴ is substituted with 0, 1 or 2 substituents independently selected from fluoro and methyl.

62. The compound of any one of claims 54 to 60, wherein the 4-10 membered heterocycle of R³⁴ is unsubstituted.

63. The compound of any one of claims 54-59, wherein R³⁴ is selected from the group consisting of:

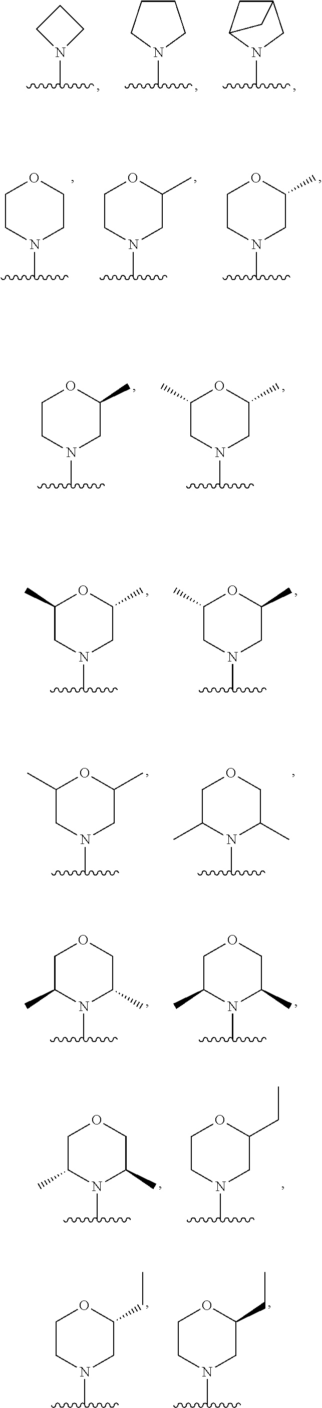

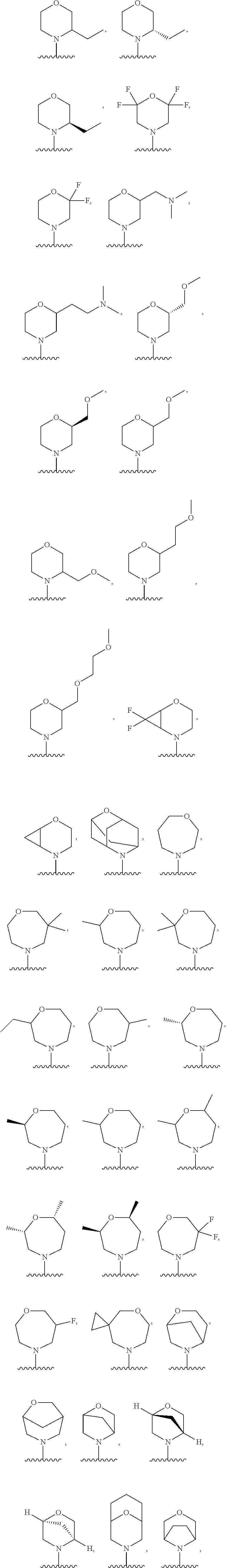

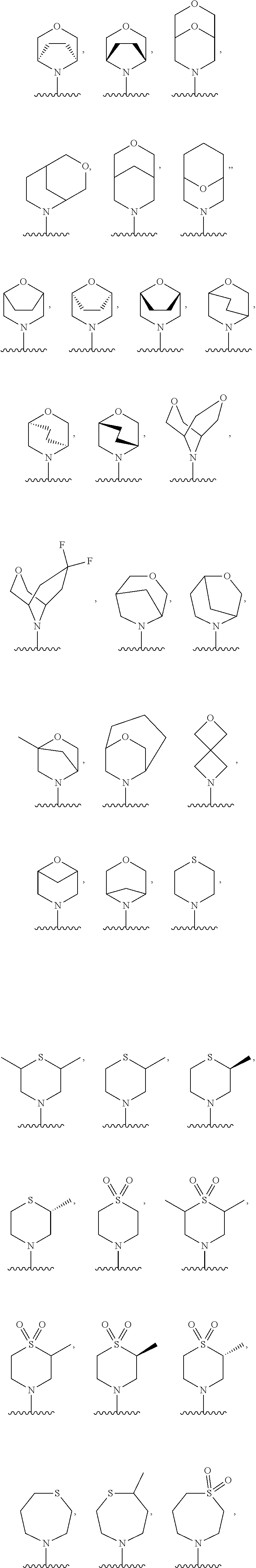

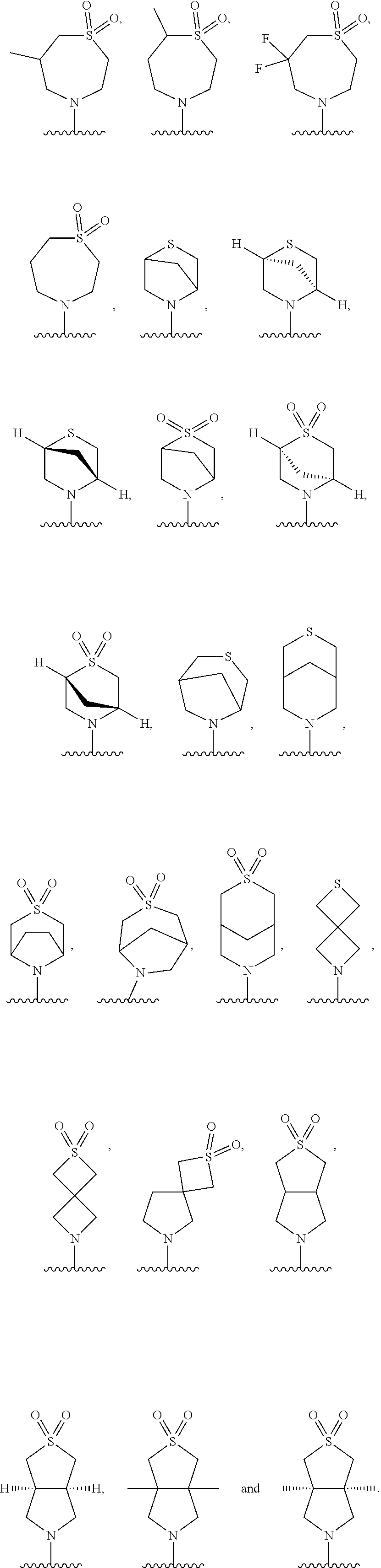

64. The compound of any one of claims 54 to 59 wherein R³⁴ is unsubstituted

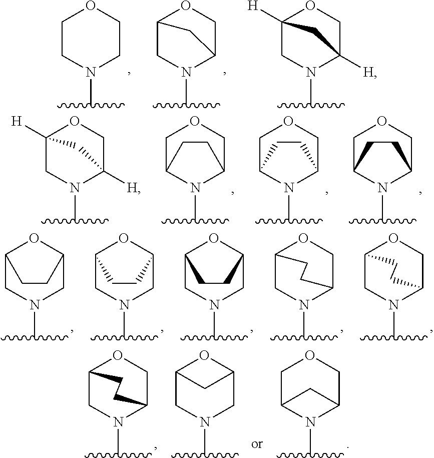

65. The compound of any one of claims 54 to 59, wherein R³⁴ is unsubstituted

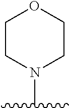 [SMILES: *N1CCOCC1]

66. The compound of any one of claims 54 to 65, wherein u is 1.

67. The compound of any one of claims 1-16 and 18-66, wherein R³⁵ is a 5-6 membered heteroaryl group containing at least one nitrogen atom, wherein the heteroaryl is substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

68. The compound of any one of claims 1-16 and 18-66, wherein R³⁵ is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

69. The compound of any one of claims 1-16 and 18-66, wherein R³⁵ is selected from the group consisting of 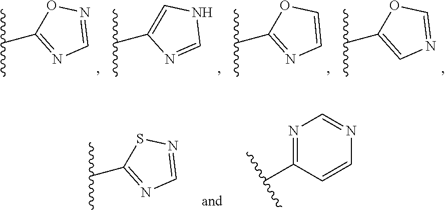
each substituted with 0, 1 or 2 substituents independently selected from halo, hydroxy, C₁-C₄ alkyl, C₁-C₄ hydroxyalkyl, C₁-C₆ alkoxy, C₁-C₄ haloalkyl, C₁-C₄ haloalkoxy, C₃-C₆ heterocyclyl optionally substituted with one or two substituents independently selected from halo and methyl, and C₃-C₆ cycloalkyl optionally substituted with one or two substituents independently selected from halo and methyl.

70. The compound of any one of claims 1-16 and 18-66, wherein R³⁵ is selected from the group consisting of:

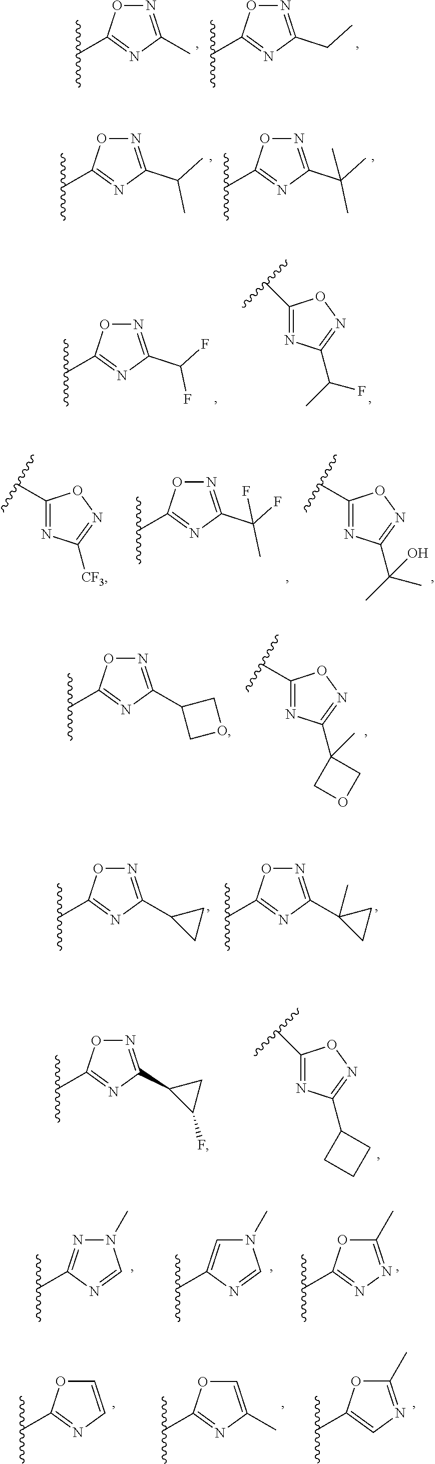

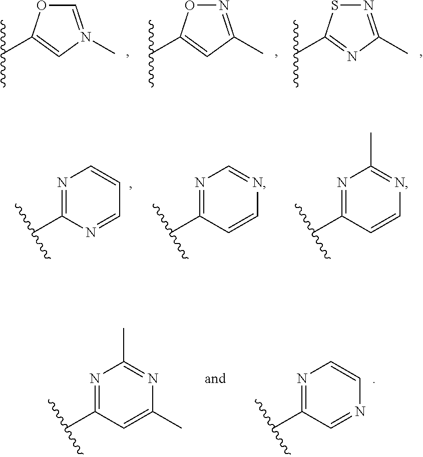

71. The compound of any one of claims 1-16 and 18-70, wherein the attachment point for R³⁵ is on a carbon atom.

72. The compound of any one of claims 1-16 and 18-66, wherein R¹⁸ is —CH₂—R³⁴.

73. The compound of any one of claims 1-16, 18-43 and 45-66, wherein R¹⁸ is R³⁴.

74. The compound of any one of claims 1-16, 18-43 and 67-73, wherein R¹⁸ is R³⁵.

75. The compound of any one of claims 1-16 and 18-44, wherein R¹⁸ is —CH₂N(CH₃).

76. The compound of any one of claims 1-16 and 18-44, wherein R¹⁸ is H.

77. The compound of any one of claims 1-16 and 18-75, wherein R¹⁸ is not H.

78. The compound of any one of claims 1-16 and 18-42, wherein R¹⁸ is selected from the group consisting of hydrogen, —COOH, —C(O)OCH₃, —C(O)OCH₂CH₃, —C(O)OCH(CH₃)₂, —C(O)N(CH₃)₂, —C(O)-cyclopropyl, —CH₂OCH₃, —CH₂N(CH₃)₂, —S(O)₂CH₃, —S(O)₂CH₂CH₃, —S(O)₂-cyclopropyl,

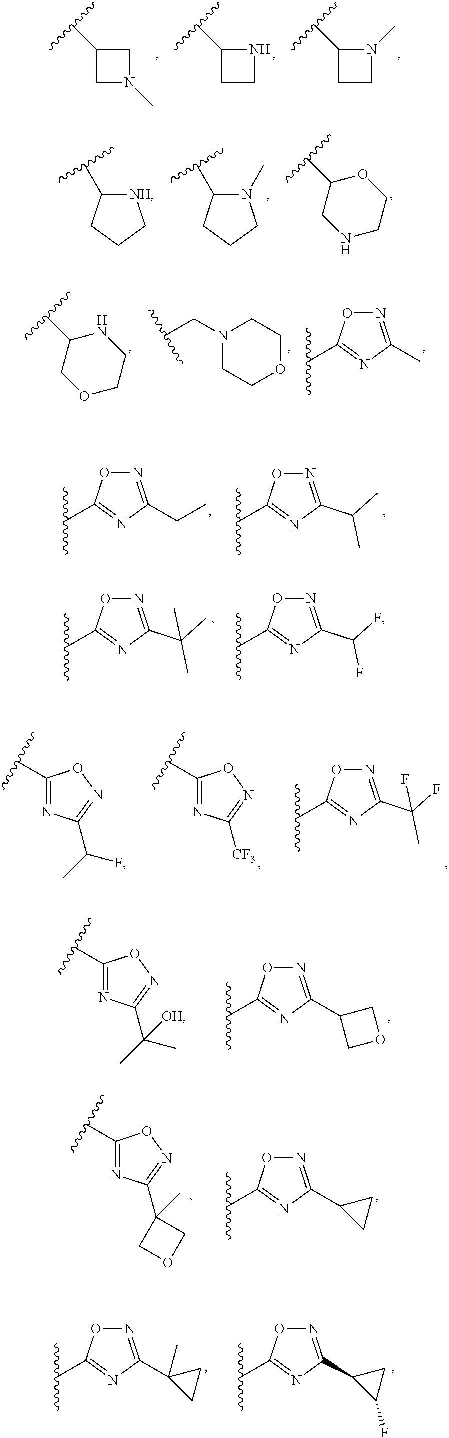

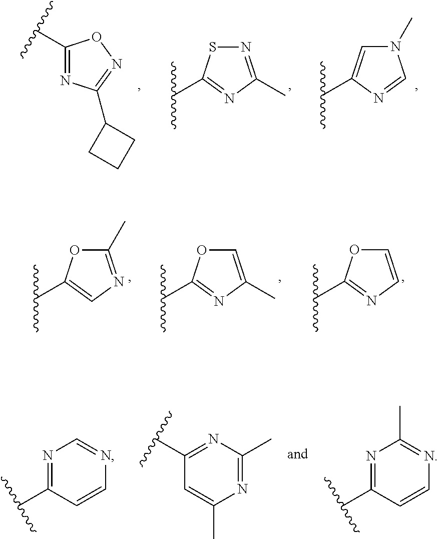

79. The compound of any one of claims 1-16 and 18-42, wherein R¹⁸ is selected from the group consisting of hydrogen, —CH₂N(CH₃)₂,

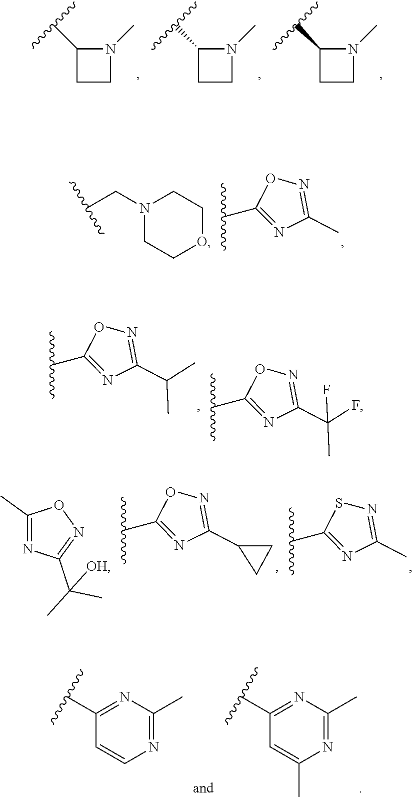

80. The compound of any one of claims 1-16 and 18-79, wherein the double bond in the —C(O)C(R¹⁹)═C(R²⁰)R¹⁸ portion of the compound is in the E configuration.

81. The compound of any one of claims 1-16, wherein R² is R^{2e}.

82. The compound of any one of claims 1-16, 18-28 and 30-81, wherein R²⁸ is methyl.

83. The compound of any one of claims 1-16, 18-28 and 30-82, wherein t is 0 or 1.

84. The compound of any one of claims 1-16, 18-28 and 30-83, wherein R³⁰ is a 4-5 membered monocyclic saturated heterocyclic group comprising one nitrogen as the sole heteroatom within the ring atoms, wherein the nitrogen ring atom of the heterocyclic group is substituted with —C(O)C≡CR³¹ and wherein the heterocyclic group is not further substituted or is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

85. The compound of any one of claims 1-16, 18-28 and 30-84 wherein the heterocyclic group of R³⁰ is not further substituted.

86. The compound of any one of claims 1-16, 18-28 and 30-84 wherein the heterocyclic group of R³⁰ is further substituted with 1 substituent selected from the group consisting of hydroxy, CN, C₁-C₄ alkyl, C₁-C₄ alkoxy, C₁-C₄ haloalkyl, C₁-C₄ cyanoalkyl, C₁-C₄ haloalkoxy, and halo.

87. The compound of any one of claims 1-16, 18-28 and 30-83, wherein the heterocyclic group of R³⁰ is selected from the group consisting of: 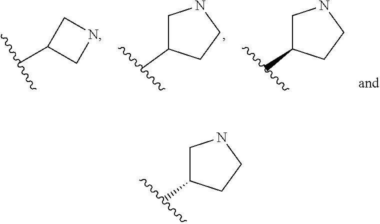
and wherein the ring nitrogen of the heterocyclic group is substituted with —C(O)C≡CR³¹ and the heterocyclic group is not further substituted, or is substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

88. The compound of claim 87, wherein the heterocyclic group of R³⁰ is not further substituted.

89. The compound of claim 87, wherein the heterocyclic group of R³⁰ is further substituted with 1 substituent selected from the group consisting of hydroxy, CN, C₁-C₄ alkyl, C₁-C₄ cyanoalkyl, and halo.

90. The compound of any one of claims 1-16, 18-28 and 30-82, wherein R²⁹ is selected from the group consisting of: 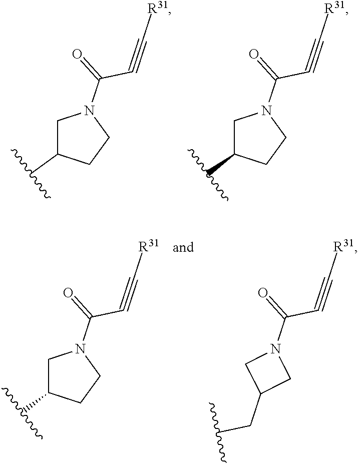
wherein the azetidine and pyrrolidine groups are not further substituted, or are substituted with one substituent selected from hydroxy, CN, C₁-C₄ alkyl, C₁-C₄ alkoxy, C₁-C₄ haloalkyl, C₁-C₄ cyanoalkyl, C₁-C₄ haloalkoxy, and halo.

91. The compound of claim 90, wherein the azetidine and pyrrolidine groups are not further substituted.

92. The compound of claim 90, wherein the azetidine and pyrrolidine groups are further substituted with 1 substituent selected from the group consisting of hydroxy, CN, C₁-C₄ alkyl, C₁-C₄ cyanoalkyl, and halo.

93. The compound of any one of claims 1-16, 18-28 and 30-92, wherein R³¹ is selected from the group consisting of —CH₂—NR³²R³³ and —CH₂—R³⁶.

94. The compound of any one of claims 1-16, 18-28 and 30-93, wherein R³² and R³³ are independently selected from methyl and ethyl.

95. The compound of any one of claims 1-16, 18-28 and 30-93, wherein R³⁶ is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

96. The compound of claim 95, wherein the monocyclic heterocycle of R³⁶ is substituted with 0 or 1 instance of methyl.

97. The compound of claim 96, wherein R³⁶ is selected from azetidinyl, pyrrolidinyl and morpholinyl substituted with 0 or 1 instance of methyl.

98. The compound of claim 96, wherein R³⁶ is azetidinyl substituted with 0 or 1 instance of methyl.

99. The compound of claim 96, wherein R³⁶ is pyrrolidinyl substituted with 0 or 1 instance of methyl.

100. The compound of claim 96, wherein R³⁶ is morpholinyl substituted with 0 or 1 instance of methyl.

101. The compound of any one of claims 1-16, 18-28 and 30-100, wherein the attachment point for R³⁶ is a carbon atom.

102. The compound of claim 101, wherein R³⁶ is selected from the group consisting of:

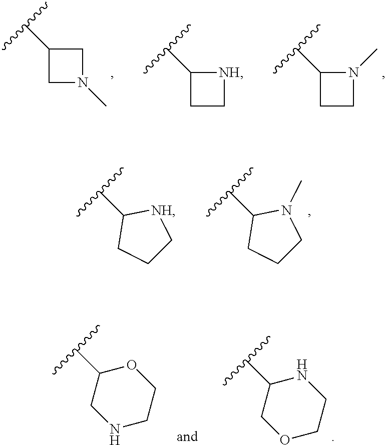

103. The compound of any one of claims 95-102, wherein p is 0.

104. The compound of any one of claims 1-16, 18-28 and 30-94, wherein R³⁶ is a 4-10 membered heterocycle containing a nitrogen atom and zero, one or two additional heteroatoms selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

105. The compound of claim 104, wherein R³⁶ is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 2-oxa-5-azabicyclo[2.2.2]octane, 7-oxa-2-azabicyclo[3.3.1]nonane, 8-oxa-3-azabicyclo[3.2.1]octane, 9-oxa-3-azabicyclo[3.3.1]nonane, 6-oxa-8-azabicyclo[3.2.2]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 1,4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

106. The compound of claim 105, wherein R³⁶ is morpholine substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

107. The compound of any one of claims 104-106, wherein the attachment point for R³⁶ is the nitrogen atom of the heterocycle.

108. The compound of claim 107 wherein the R³⁶ is selected from the group consisting of: 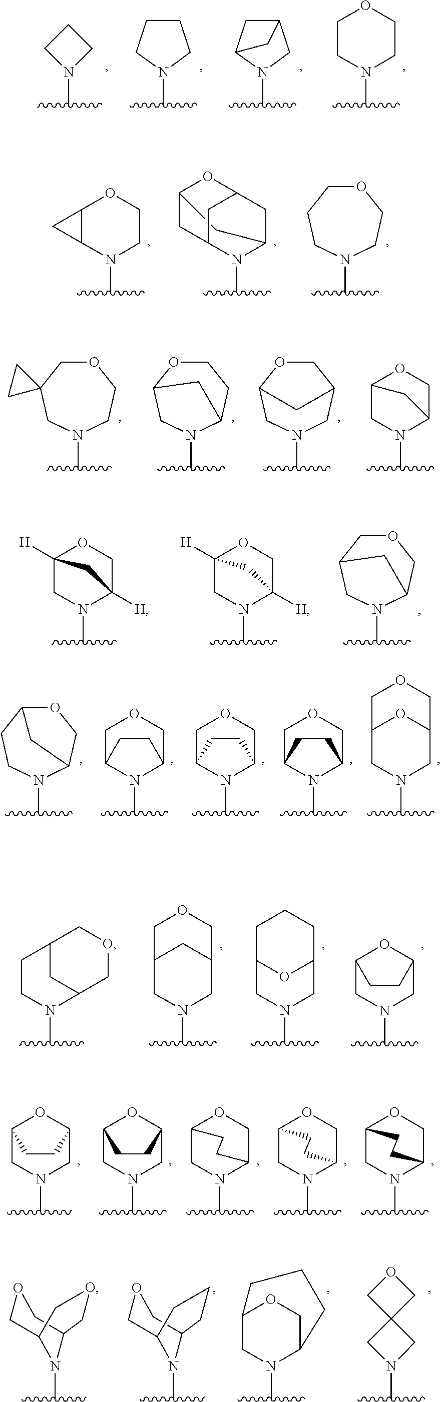 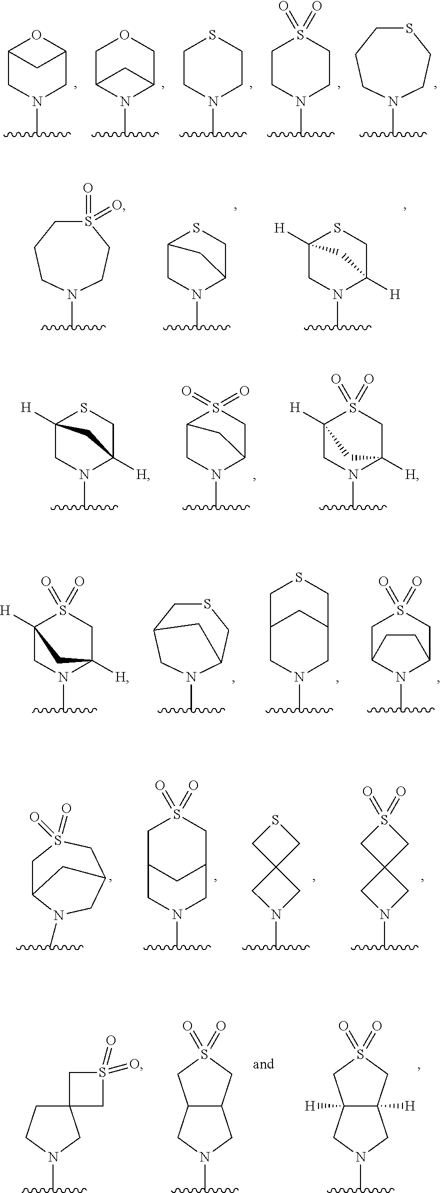
each substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

109. The compound of claim 108, wherein R³⁶ is 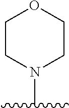 [SMILES: *N1CCOCC1]
substituted with 0, 1, 2, 3 or 4 substituents independently selected from halo, hydroxy, C₁-C₄ alkyl, C₁-C₆ aminoalkyl, C₁-C₆ alkoxy, C₁-C₄ haloalkyl, C₁-C₄ haloalkoxy and C₂-C₃ alkynyl.

110. The compound of any one of claims 104-109, wherein the 4-10 membered heterocycle of R³⁶ is unsubstituted.

111. The compound of any one of claims 104-108, wherein R³⁶ is selected from the group consisting of:

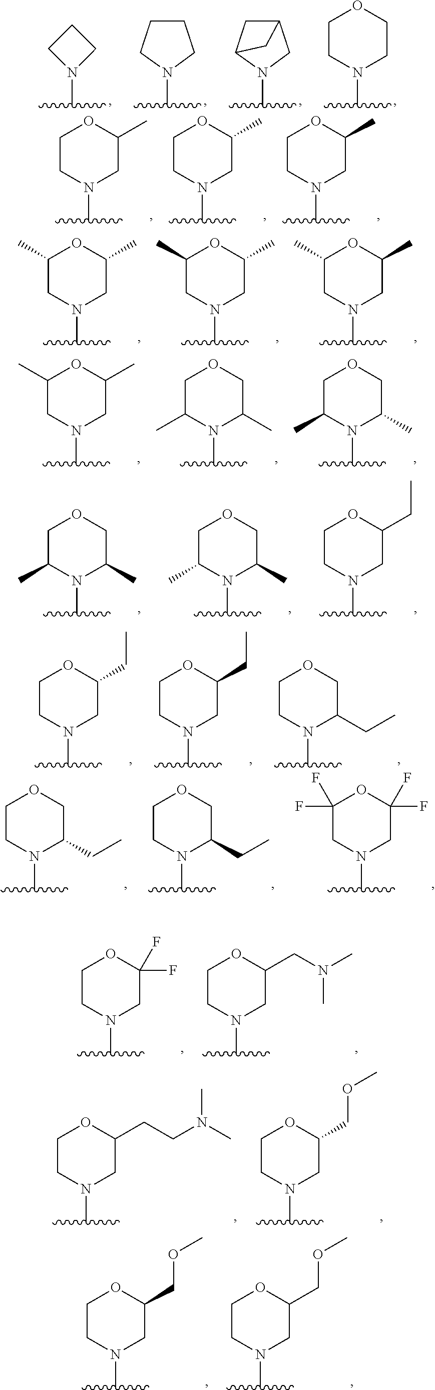

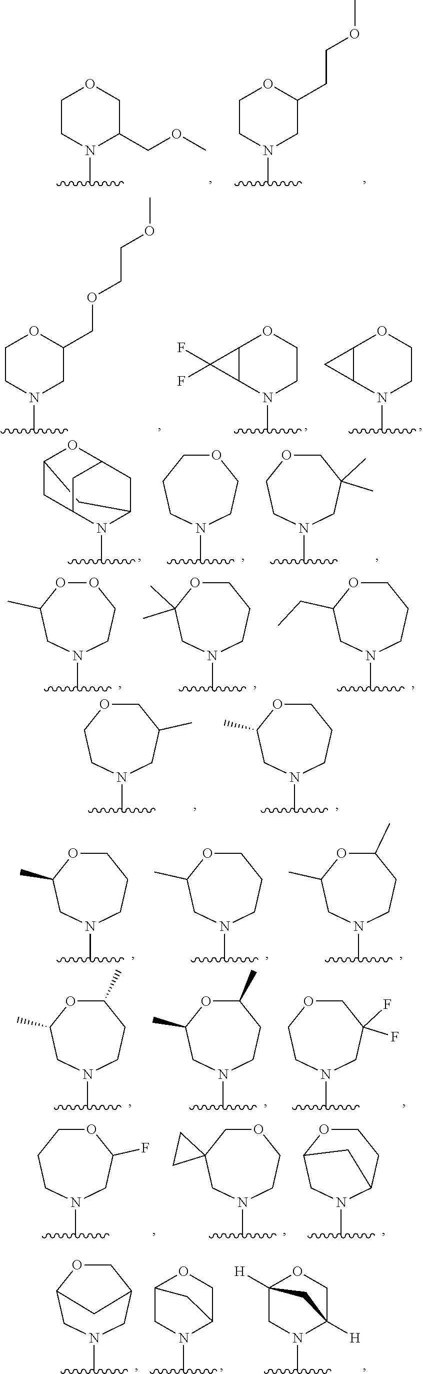

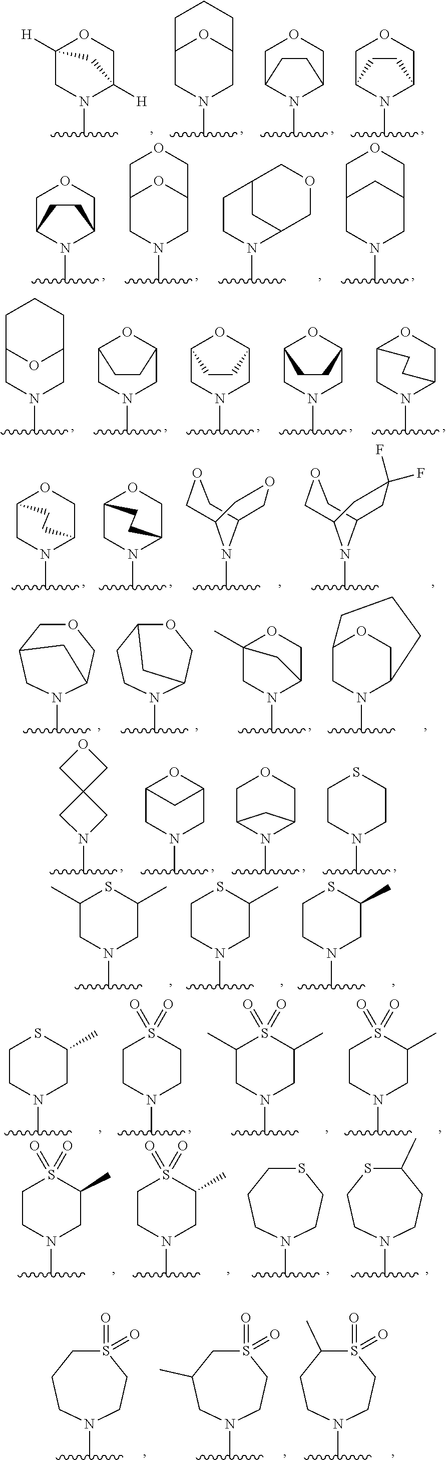

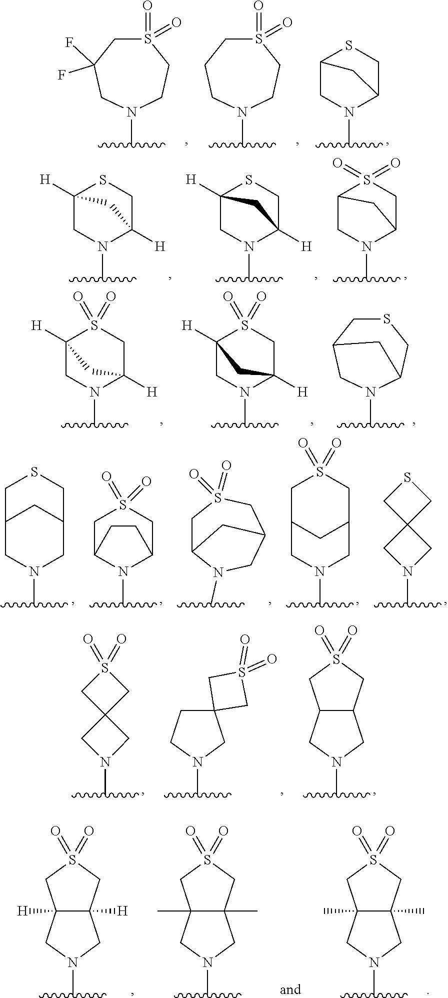

112. The compound of any one of claims 104-110, wherein R³⁶ is unsubstituted

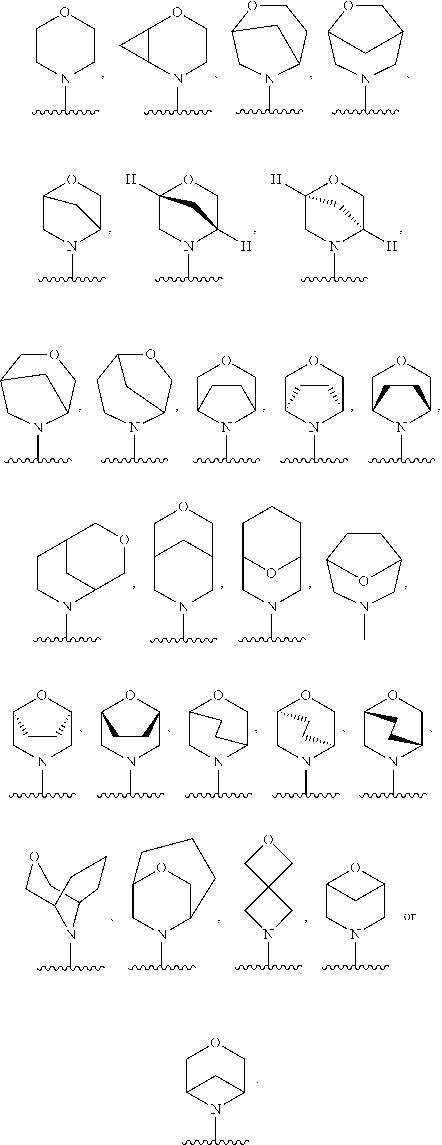

113. The compound of any one of claims 104-110, wherein R³⁶ is unsubstituted

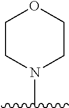 [SMILES: *N1CCOCC1]

114. The compound of any one of claims 104-113, wherein p is 1.

115. The compound of any one of claims 1-16, 18-28 and 30-92, wherein R³¹ is selected from the group consisting of:

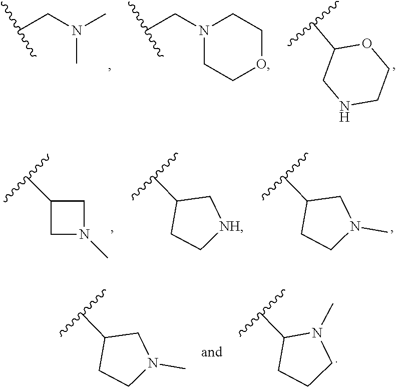

116. The compound of any one of claims 1-16, 18-28 and 30-92, wherein R³¹ is selected from the group consisting of:

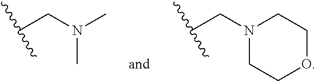

117. The compound of claim 1, selected from the group consisting of: 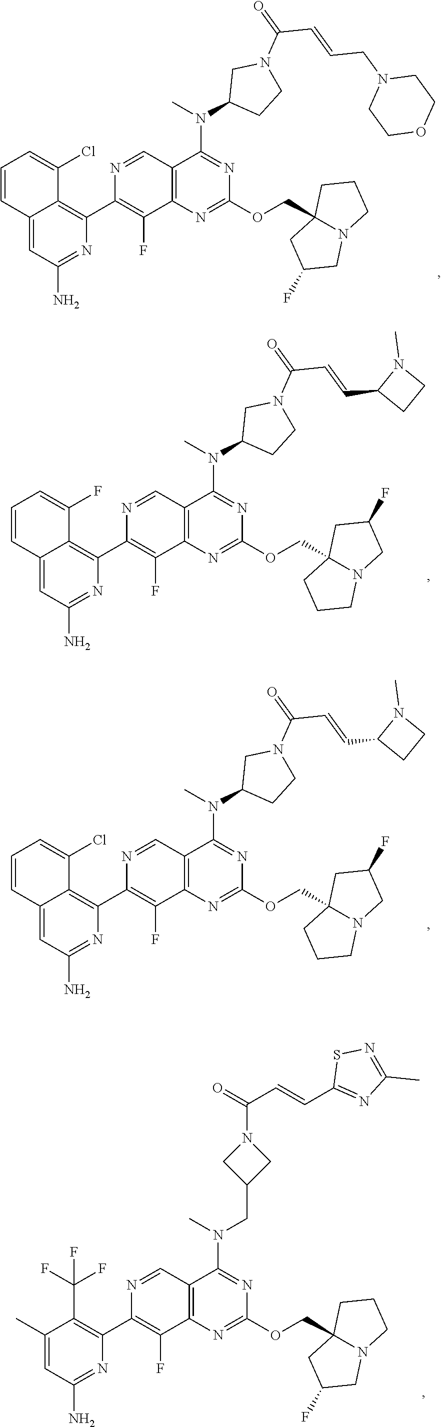 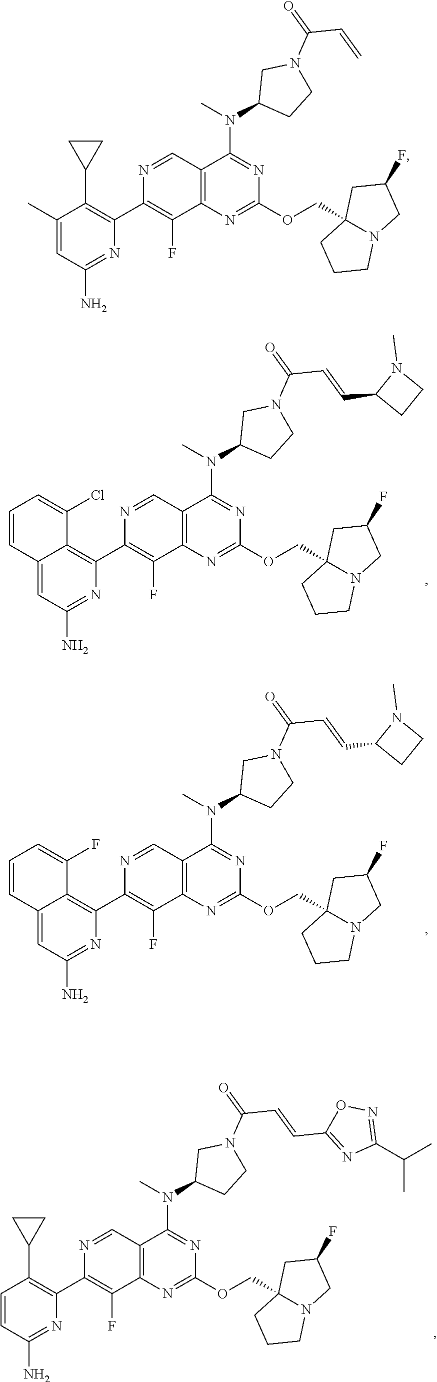 [SMILES: C=CC(=O)N1CC[C@@H](N(C)c2nc(OC[C@@]34CCCN3C[C@H](F)C4)nc3c(F)c(-c4nc(N)cc(C)c4C4CC4)ncc23)C1 ; CN1CC[C@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1] 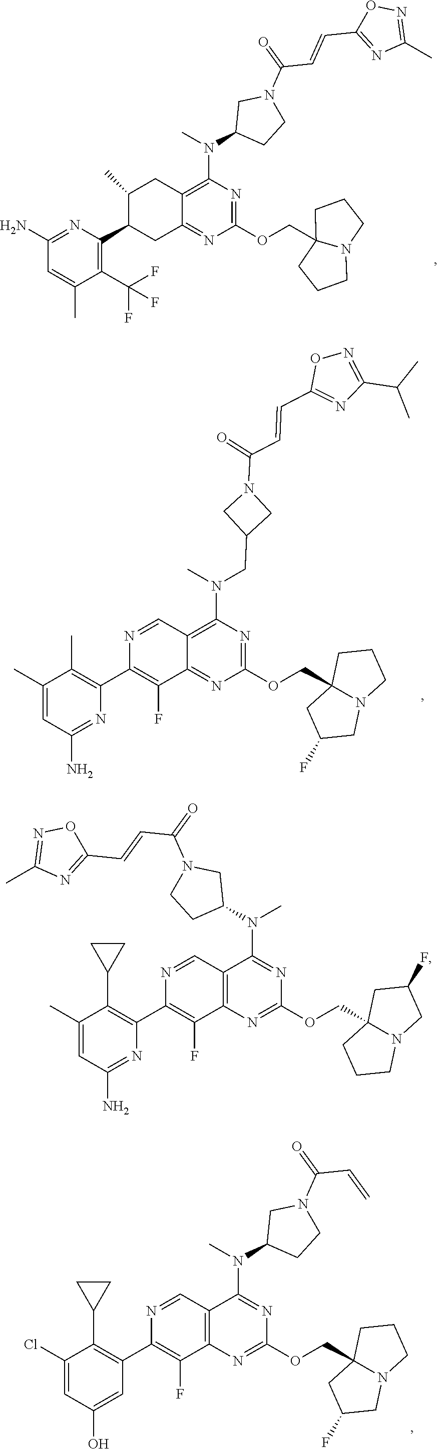 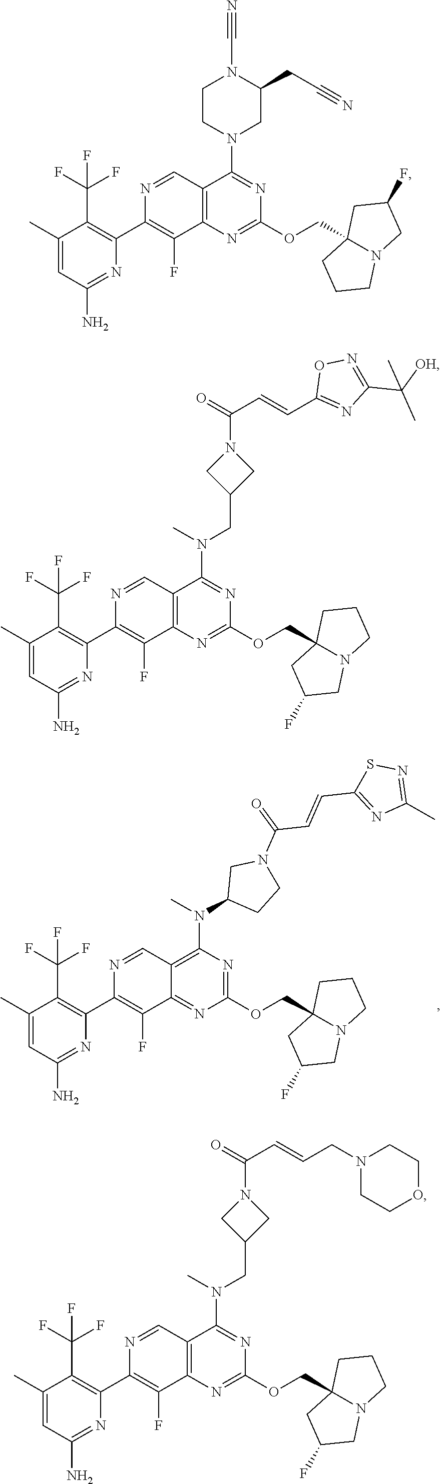 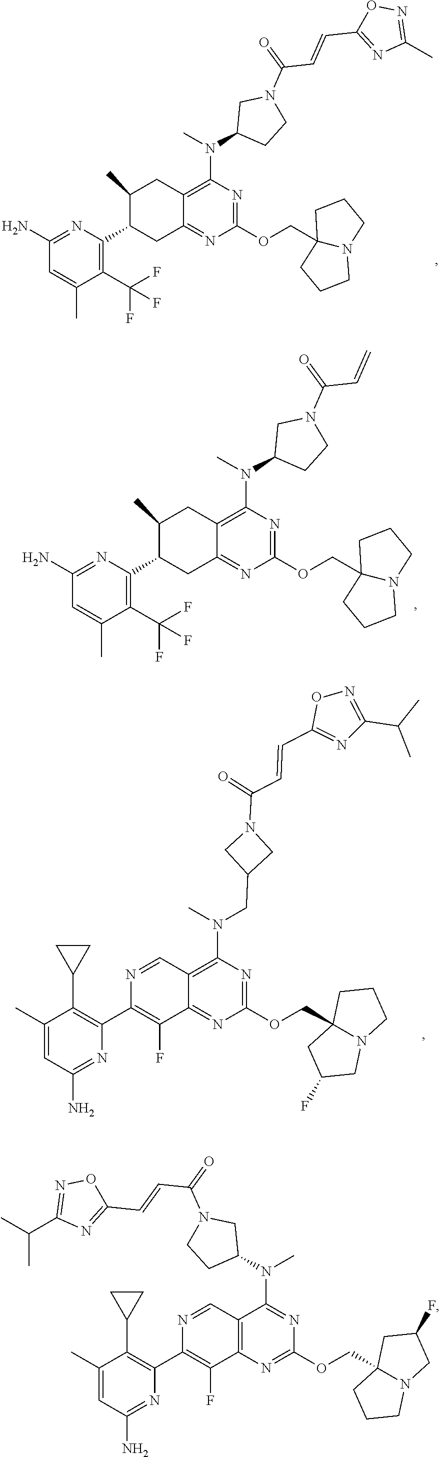 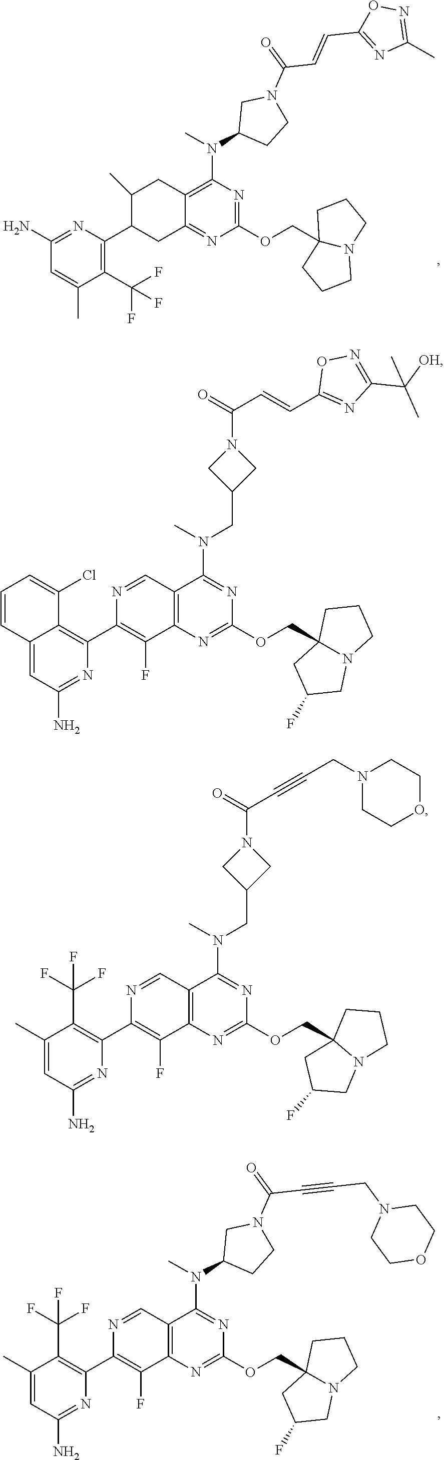 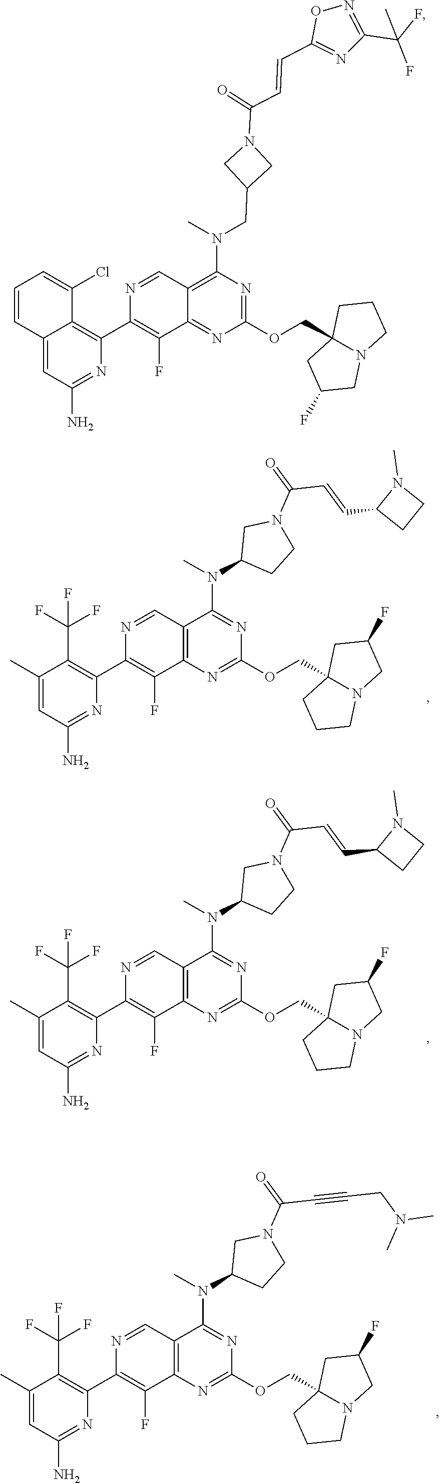 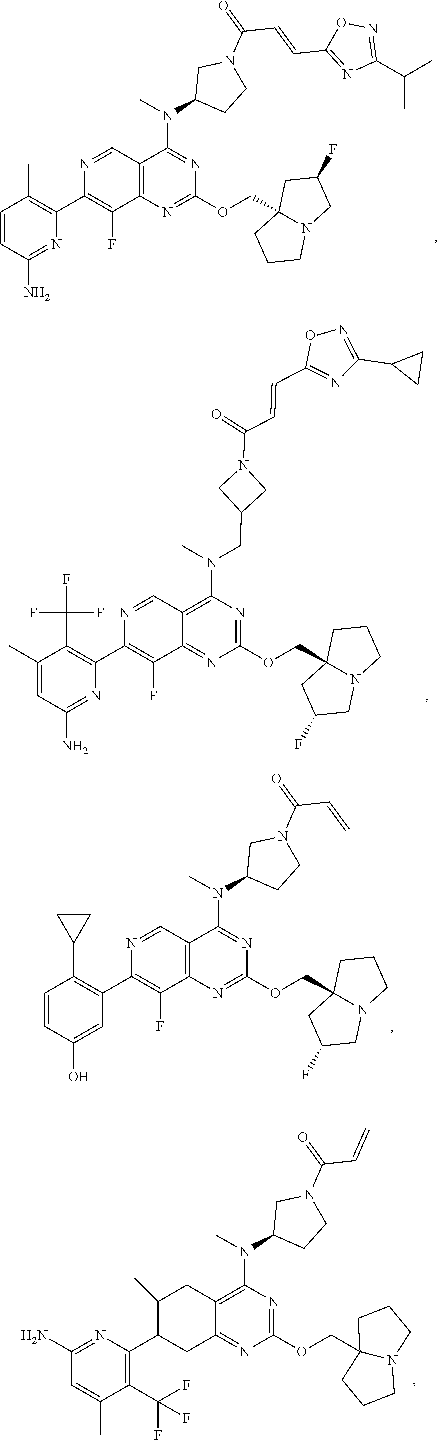 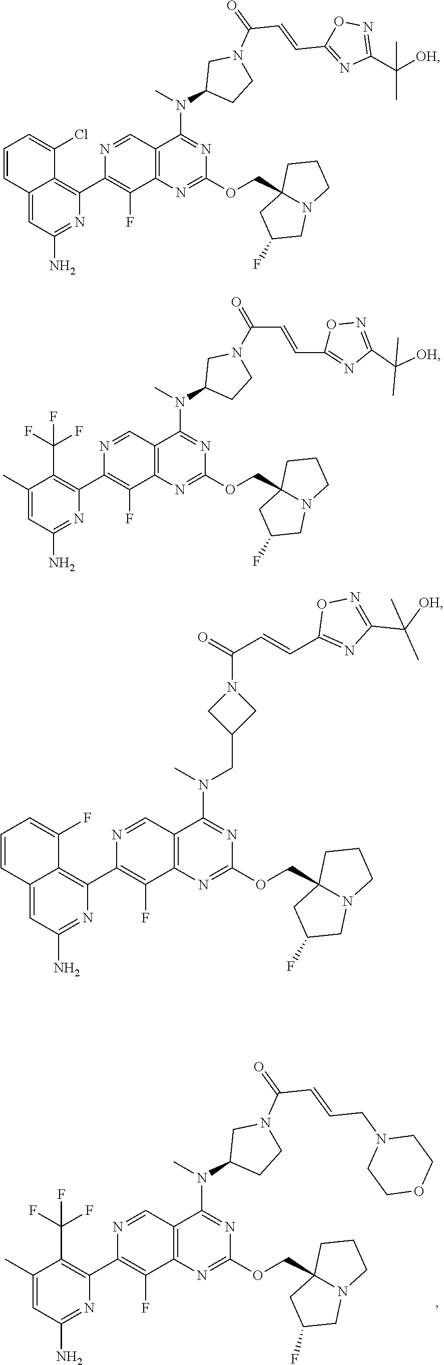 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc(-c2ncc3c(N(C)[C@@H]4CCN(C(=O)/C=C/c5nc(C(C)(C)O)no5)C4)nc(OC[C@@]45CCCN4C[C@H](F)C5)nc3c2F)c1C(F)(F)F ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(F)c34)ncc12 ; Cc1cc(N)nc(-c2ncc3c(N(C)[C@@H]4CCN(C(=O)/C=C/CN5CCOCC5)C4)nc(OC[C@@]45CCCN4C[C@H](F)C5)nc3c2F)c1C(F)(F)F] 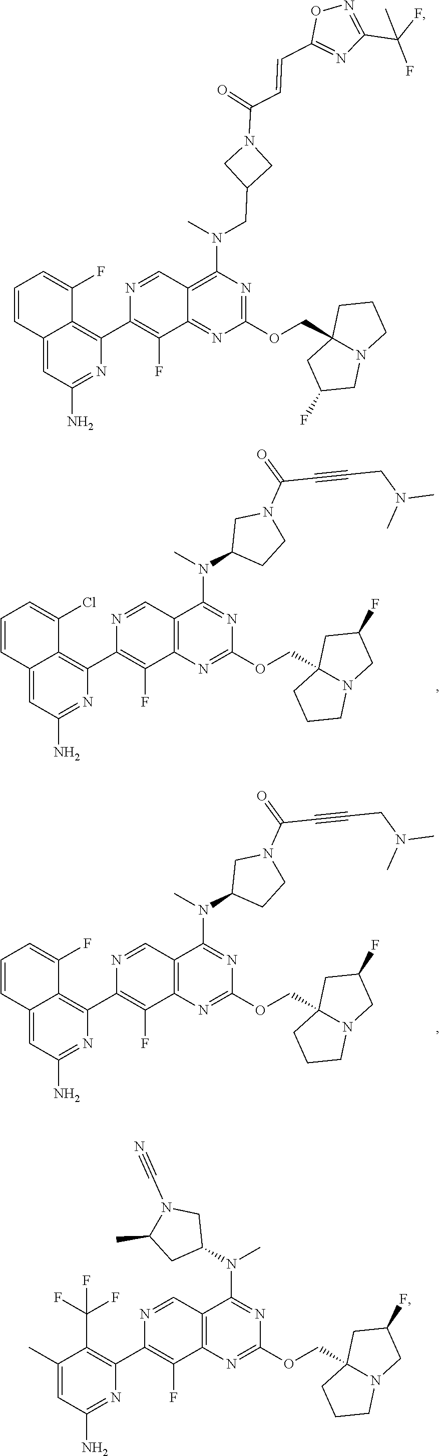 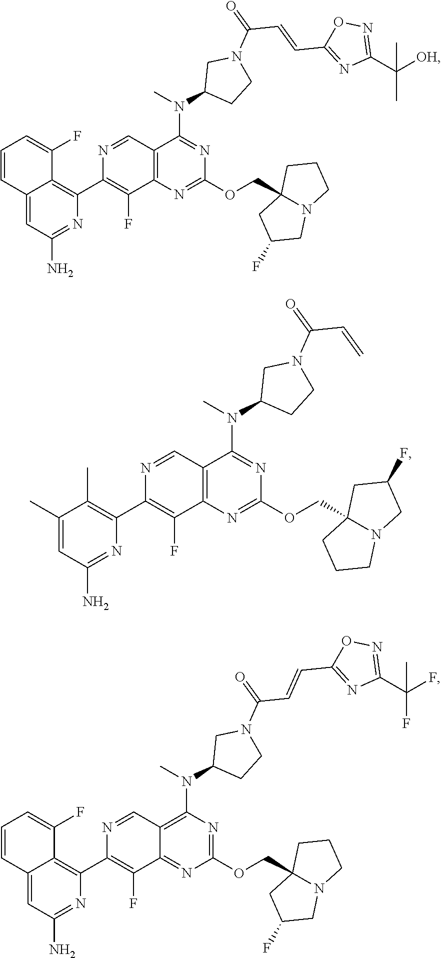 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1 ; C=CC(=O)N1CC[C@@H](N(C)c2nc(OC[C@@]34CCCN3C[C@H](F)C4)nc3c(F)c(-c4nc(N)cc(C)c4C)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1] 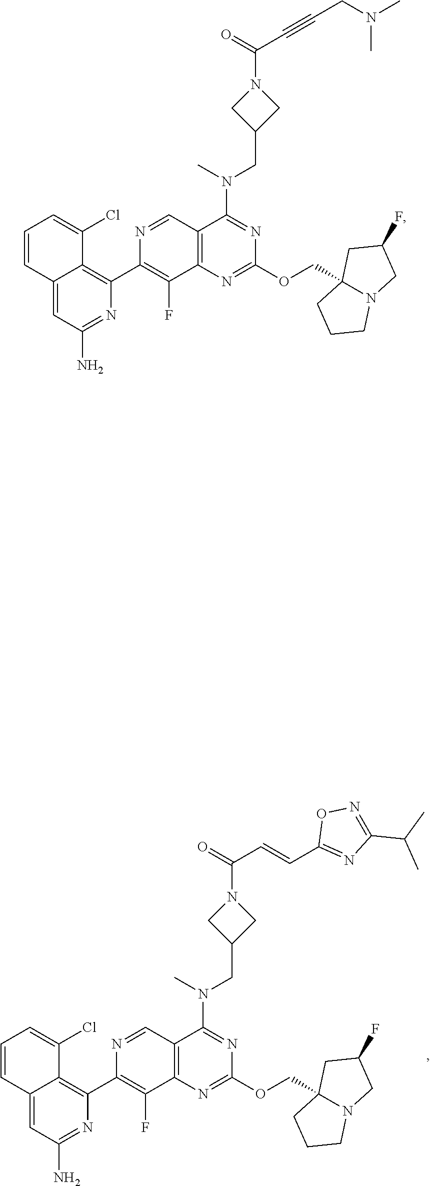 [SMILES: CN(C)CC#CC(=O)N1CC(CN(C)c2nc(OC[C@@]34CCCN3C[C@H](F)C4)nc3c(F)c(-c4nc(N)cc5cccc(Cl)c45)ncc23)C1 ; CC(C)c1noc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1] 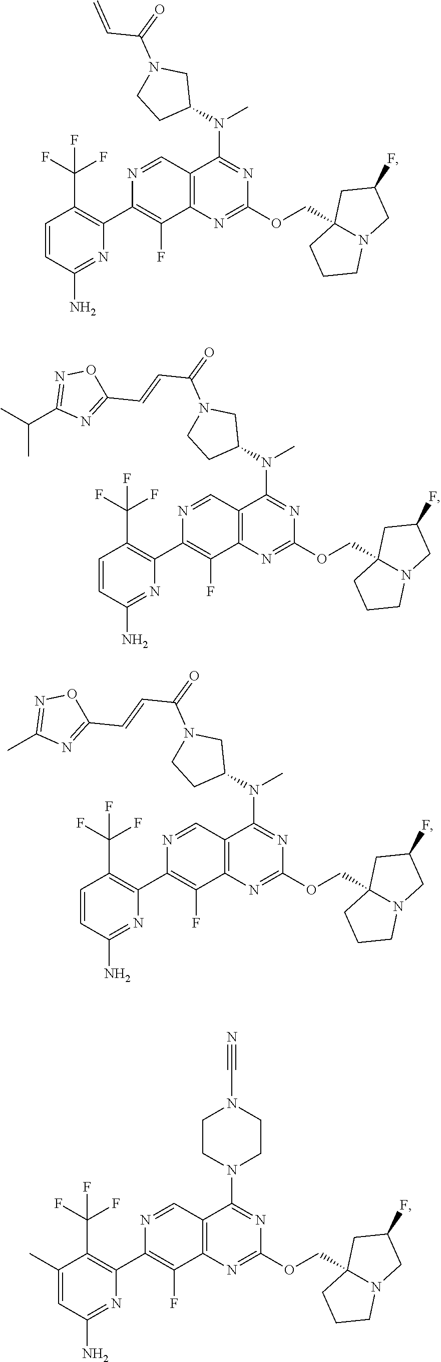 [SMILES: C=CC(=O)N1CC[C@@H](N(C)c2nc(OC[C@@]34CCCN3C[C@H](F)C4)nc3c(F)c(-c4nc(N)ccc4C(F)(F)F)ncc23)C1 ; CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1cc(N)nc(-c2ncc3c(N4CCN(C#N)CC4)nc(OC[C@@]45CCCN4C[C@H](F)C5)nc3c2F)c1C(F)(F)F] 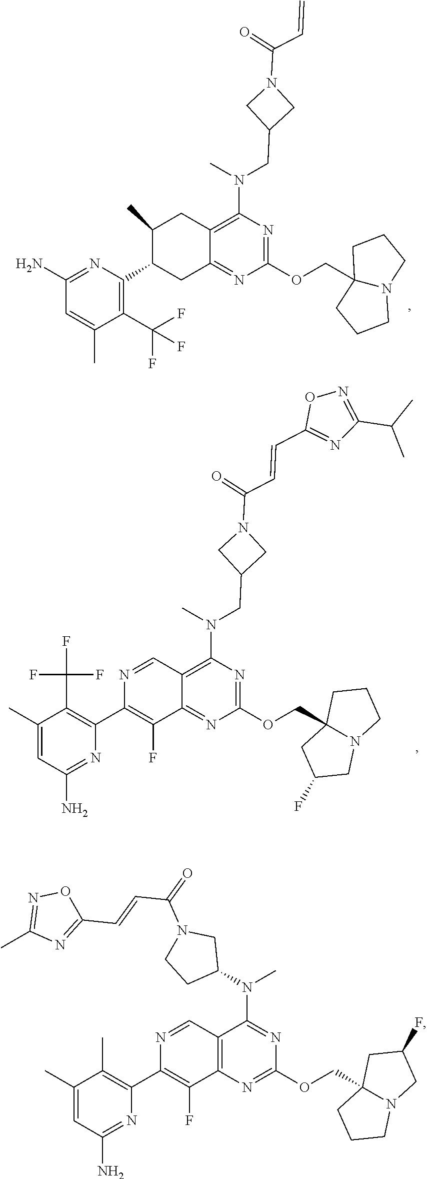 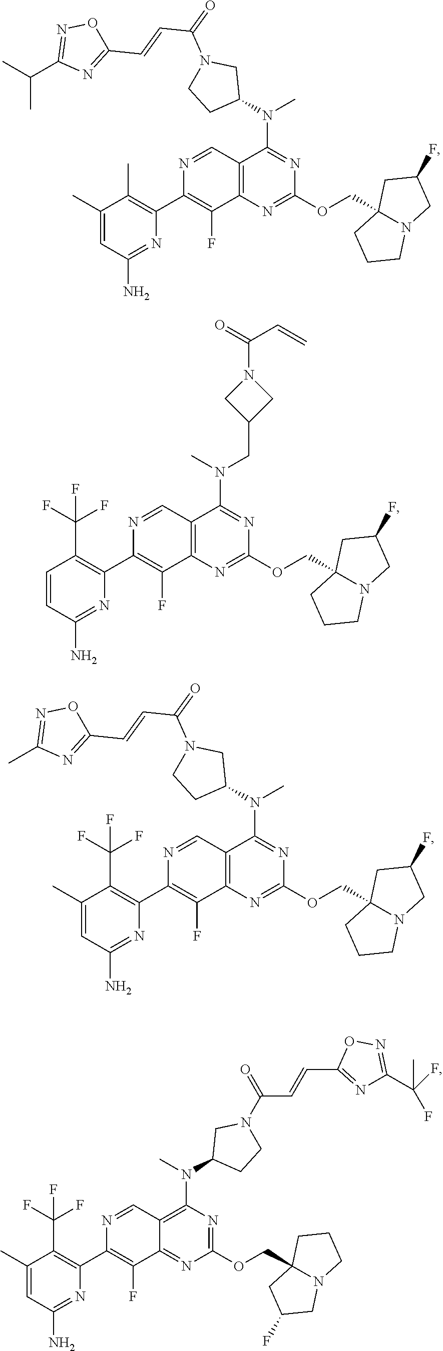 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C ; C=CC(=O)N1CC(CN(C)c2nc(OC[C@@]34CCCN3C[C@H](F)C4)nc3c(F)c(-c4nc(N)ccc4C(F)(F)F)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1 ; Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F] 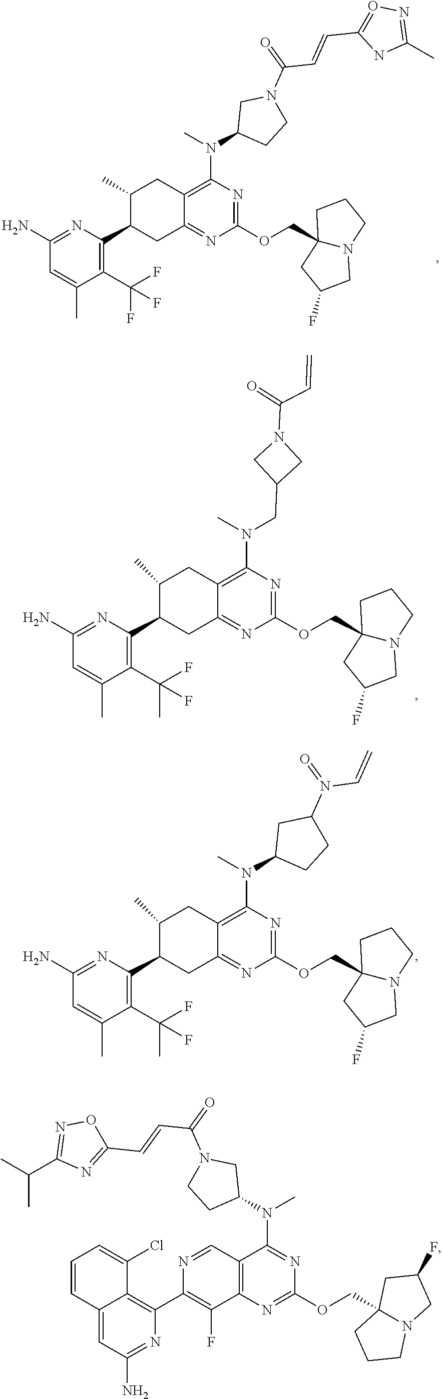 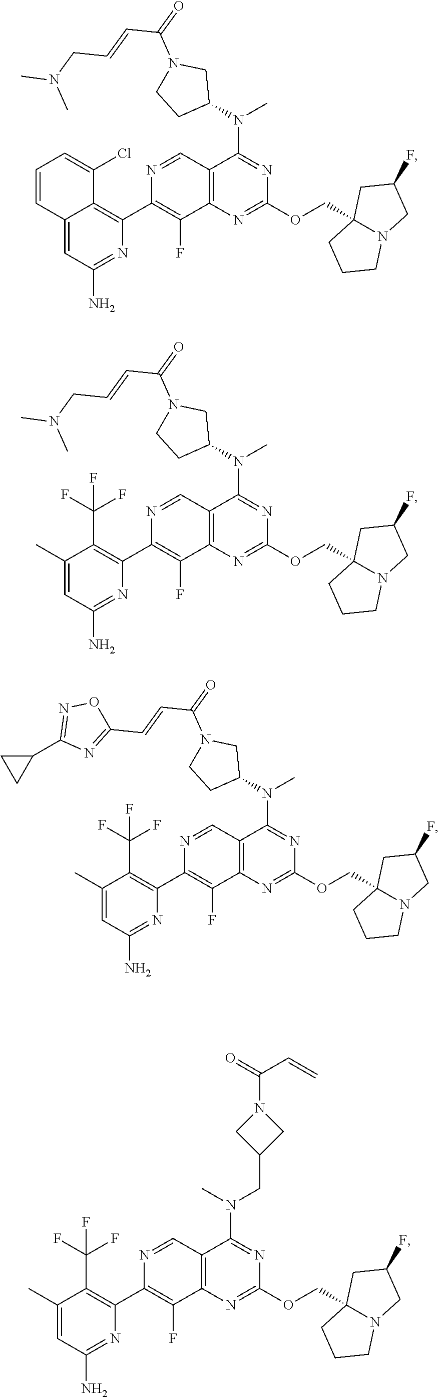 [SMILES: CN(C)C/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F ; C=CC(=O)N1CC(CN(C)c2nc(OC[C@@]34CCCN3C[C@H](F)C4)nc3c(F)c(-c4nc(N)cc(C)c4C(F)(F)F)ncc23)C1] 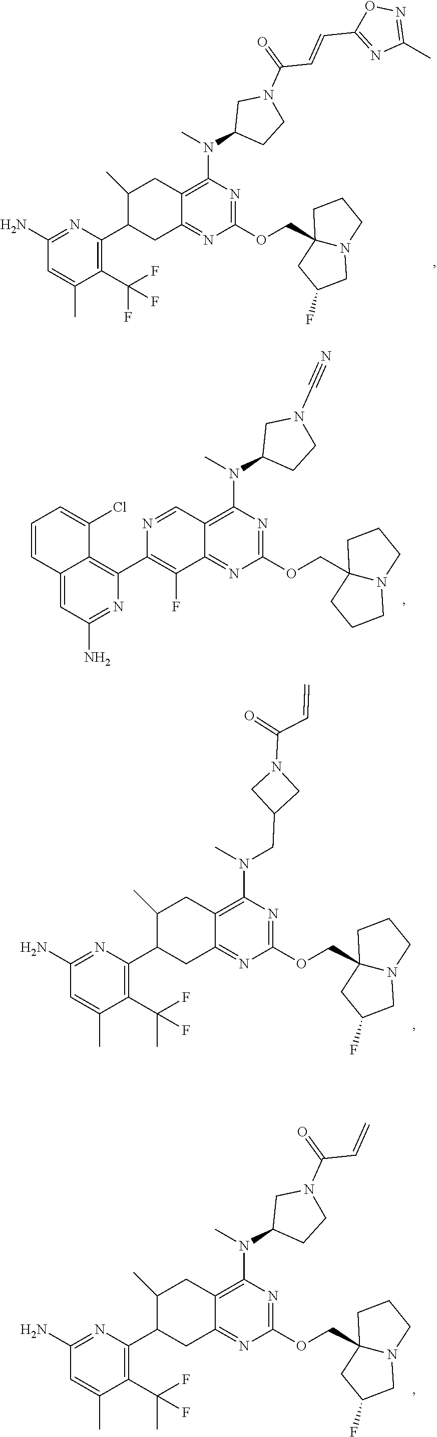 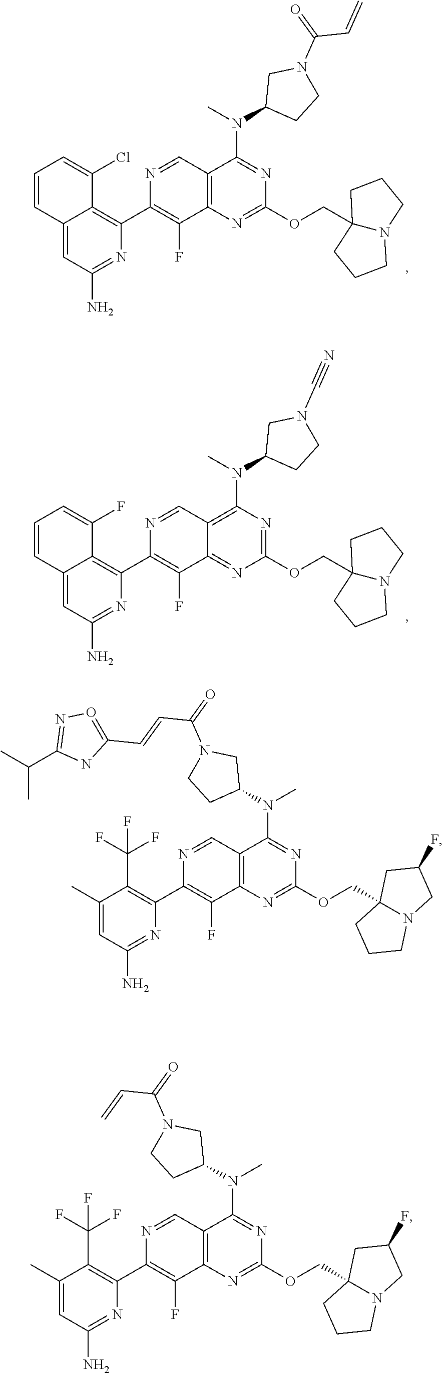 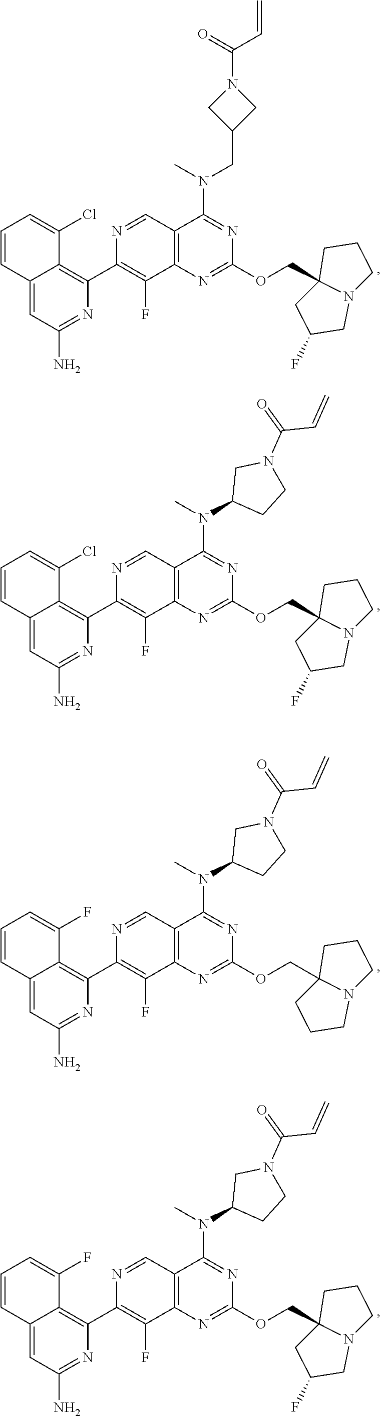 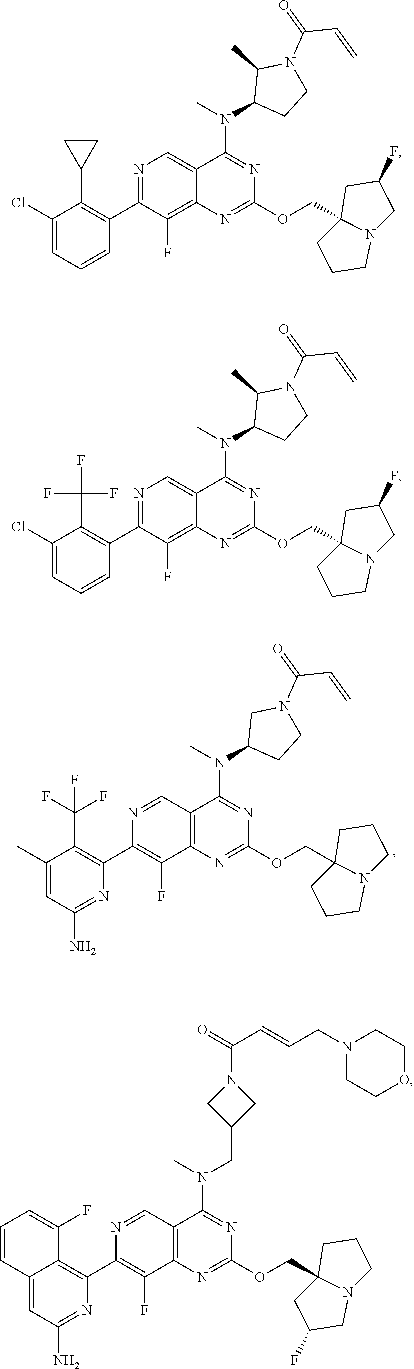 [SMILES: C=CC(=O)N1CC[C@@H](N(C)c2nc(OC[C@@]34CCCN3C[C@H](F)C4)nc3c(F)c(-c4cccc(Cl)c4C4CC4)ncc23)[C@H]1C ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C ; C=CC(=O)N1CC[C@@H](N(C)c2nc(OCC34CCCN3CCC4)nc3c(F)c(-c4nc(N)cc(C)c4C(F)(F)F)ncc23)C1 ; CN(CC1CN(C(=O)/C=C/CN2CCOCC2)C1)c1nc(OC[C@@]23CCCN2C[C@H](F)C3)nc2c(F)c(-c3nc(N)cc4cccc(F)c34)ncc12] 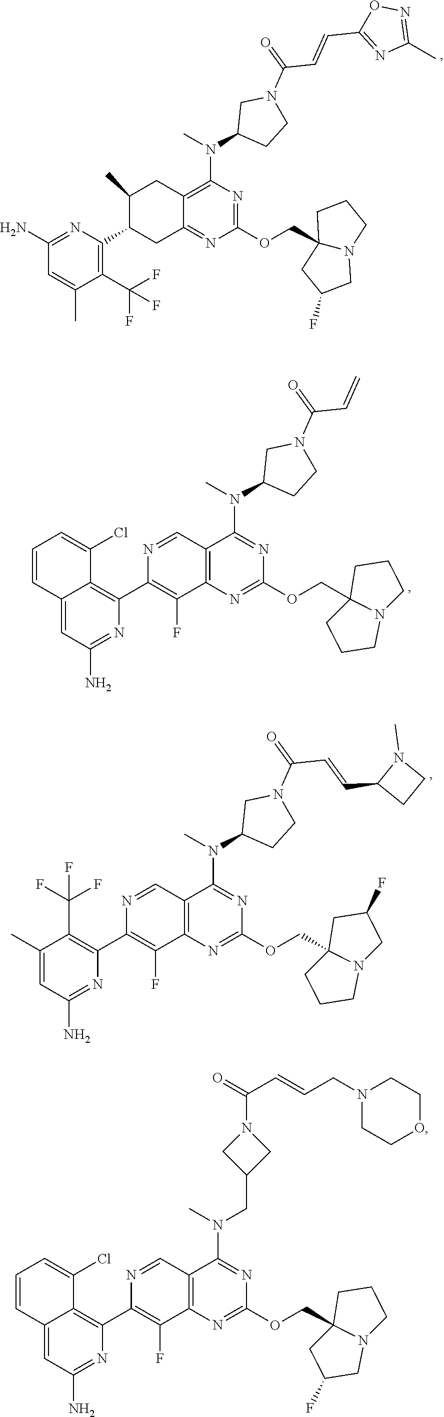 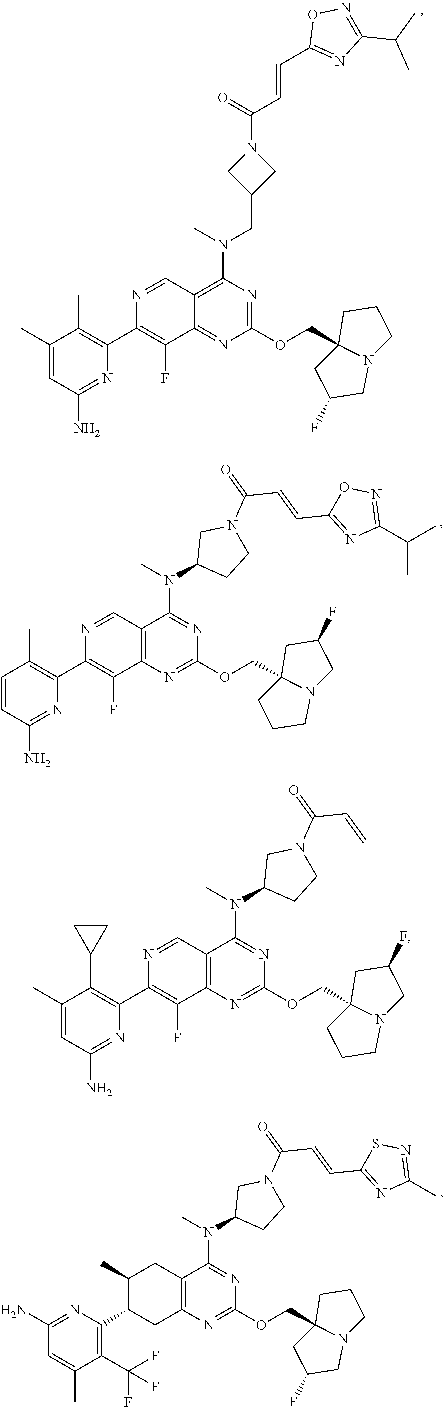 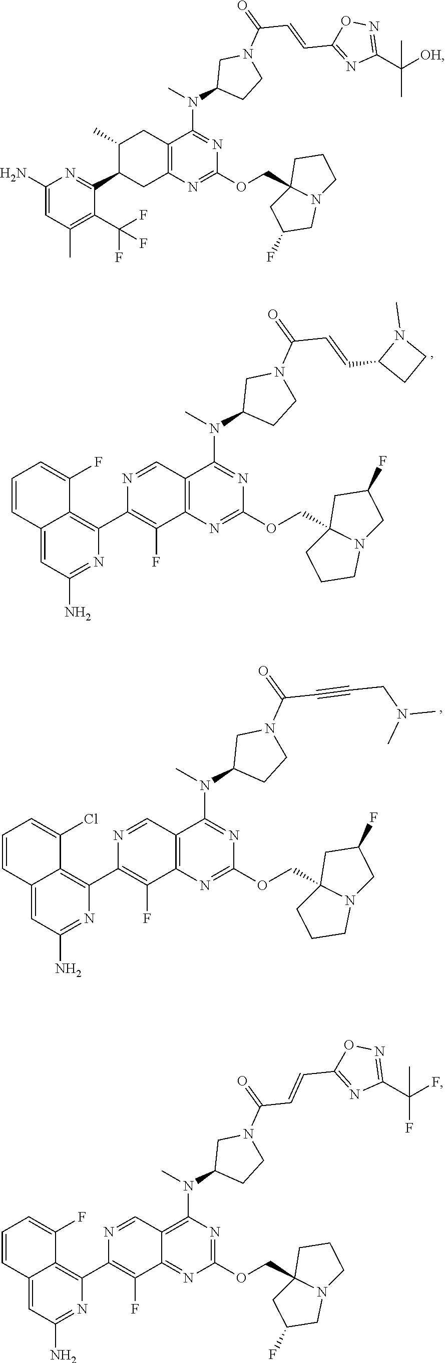 [SMILES: Cc1cc(N)nc([C@@H]2Cc3nc(OC[C@@]45CCCN4C[C@H](F)C5)nc(N(C)[C@@H]4CCN(C(=O)/C=C/c5nc(C(C)(C)O)no5)C4)c3C[C@H]2C)c1C(F)(F)F ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; CN(C)CC#CC(=O)N1CC[C@@H](N(C)c2nc(OC[C@@]34CCCN3C[C@H](F)C4)nc3c(F)c(-c4nc(N)cc5cccc(Cl)c45)ncc23)C1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(F)F)no2)C1] 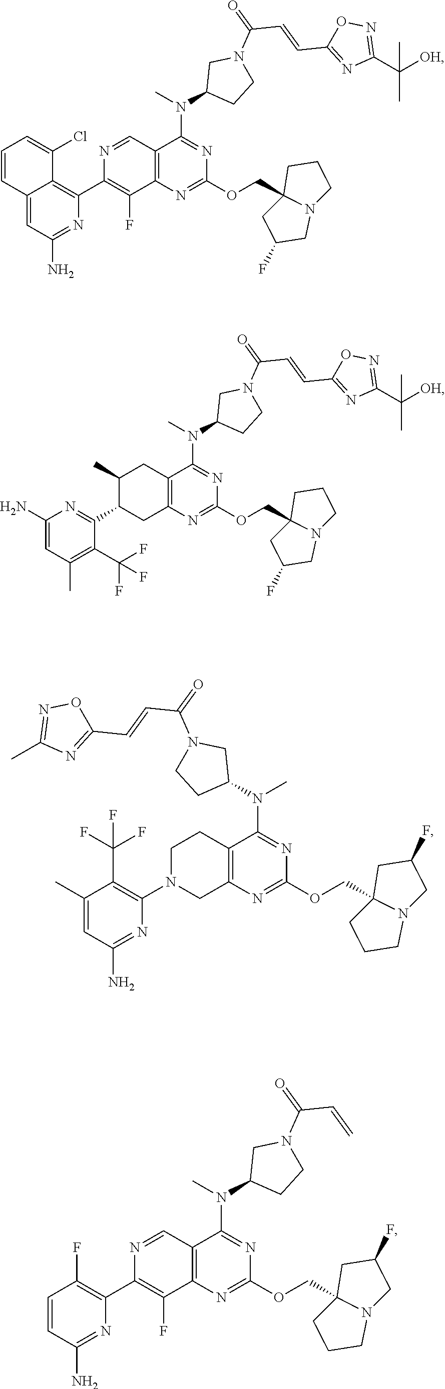 [SMILES: CN(c1nc(OC[C@@]23CCCN2C[C@H](F)C3)nc2c(F)c(-c3nc(N)cc4cccc(Cl)c34)ncc12)[C@@H]1CCN(C(=O)/C=C/c2nc(C(C)(C)O)no2)C1 ; Cc1cc(N)nc([C@H]2Cc3nc(OC[C@@]45CCCN4C[C@H](F)C5)nc(N(C)[C@@H]4CCN(C(=O)/C=C/c5nc(C(C)(C)O)no5)C4)c3C[C@@H]2C)c1C(F)(F)F ; Cc1noc(/C=C/C(=O)N2CC[C@@H](N(C)c3nc(OC[C@@]45CCCN4C[C@H](F)C5)nc4c3CCN(c3nc(N)cc(C)c3C(F)(F)F)C4)C2)n1 ; C=CC(=O)N1CC[C@@H](N(C)c2nc(OC[C@@]34CCCN3C[C@H](F)C4)nc3c(F)c(-c4nc(N)ccc4F)ncc23)C1] 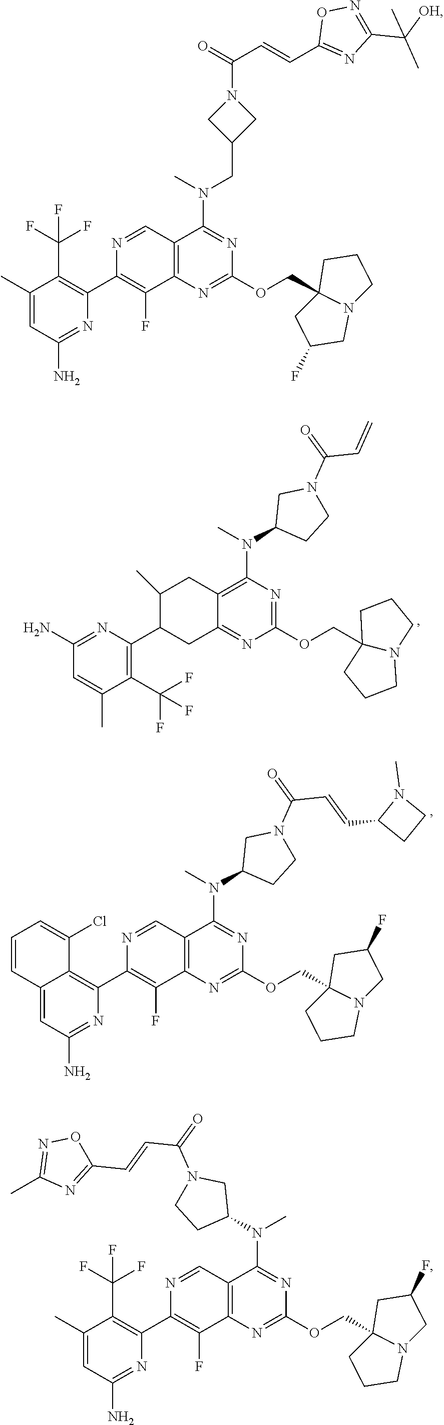 [SMILES: Cc1cc(N)nc(-c2ncc3c(N(C)CC4CN(C(=O)/C=C/c5nc(C(C)(C)O)no5)C4)nc(OC[C@@]45CCCN4C[C@H](F)C5)nc3c2F)c1C(F)(F)F ; C=CC(=O)N1CC[C@@H](N(C)c2nc(OCC34CCCN3CCC4)nc3c2CC(C)C(c2nc(N)cc(C)c2C(F)(F)F)C3)C1 ; CN1CC[C@@H]1/C=C/C(=O)N1CC[C@@H](N(C)c2nc(OC[C@@]34CCCN3C[C@H](F)C4)nc3c(F)c(-c4nc(N)cc5cccc(Cl)c45)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C(F)(F)F)ncc34)C2)n1] 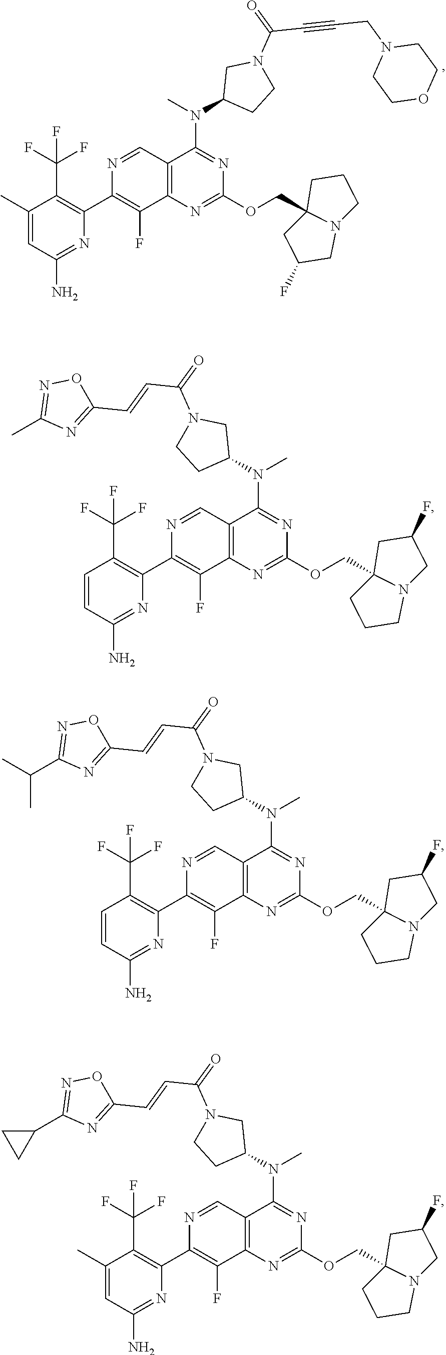 [SMILES: Cc1cc(N)nc(-c2ncc3c(N(C)[C@@H]4CCN(C(=O)C#CCN5CCOCC5)C4)nc(OC[C@@]45CCCN4C[C@H](F)C5)nc3c2F)c1C(F)(F)F ; Cc1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C(F)(F)F)ncc34)C2)n1 ; Cc1cc(N)nc(-c2ncc3c(N(C)[C@@H]4CCN(C(=O)/C=C/c5nc(C6CC6)no5)C4)nc(OC[C@@]45CCCN4C[C@H](F)C5)nc3c2F)c1C(F)(F)F] 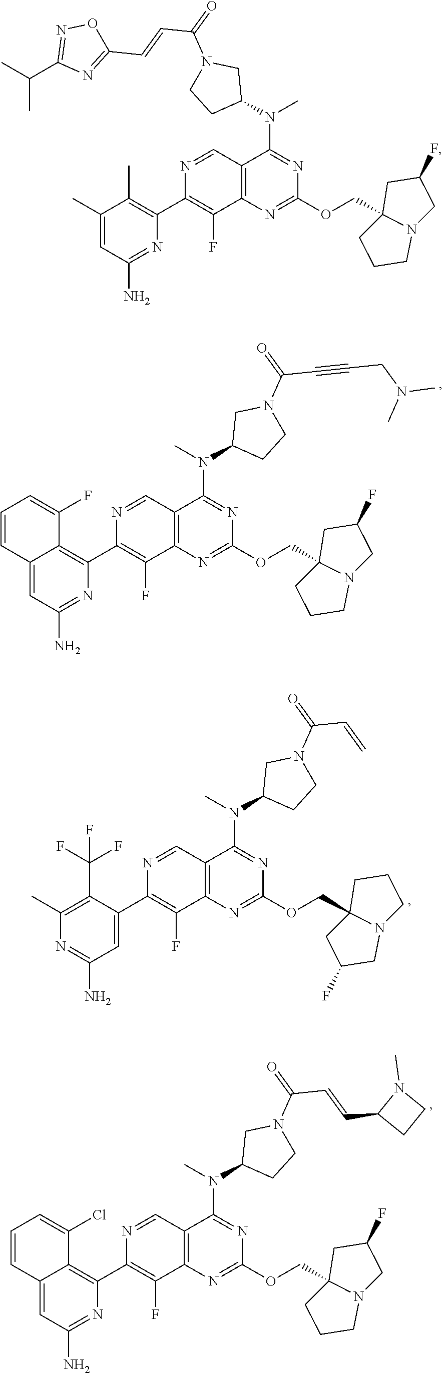 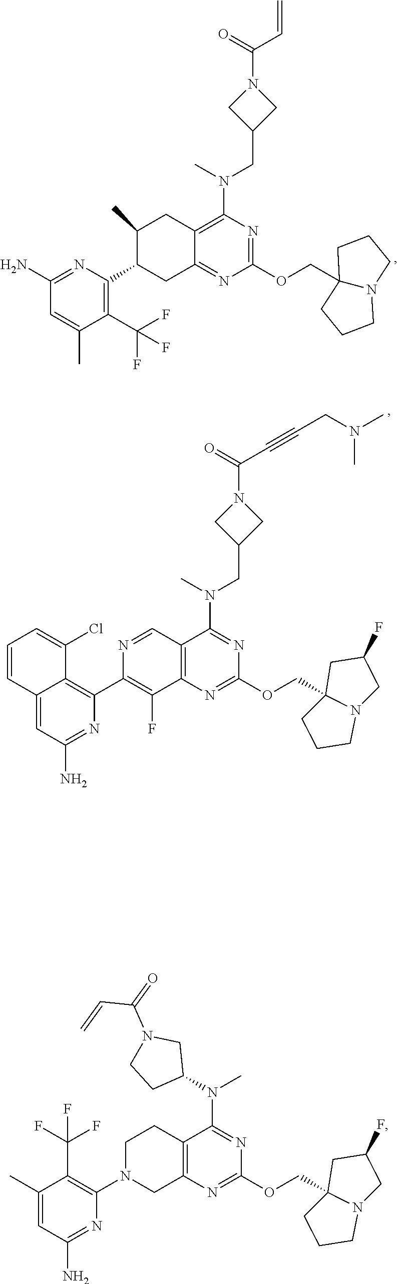 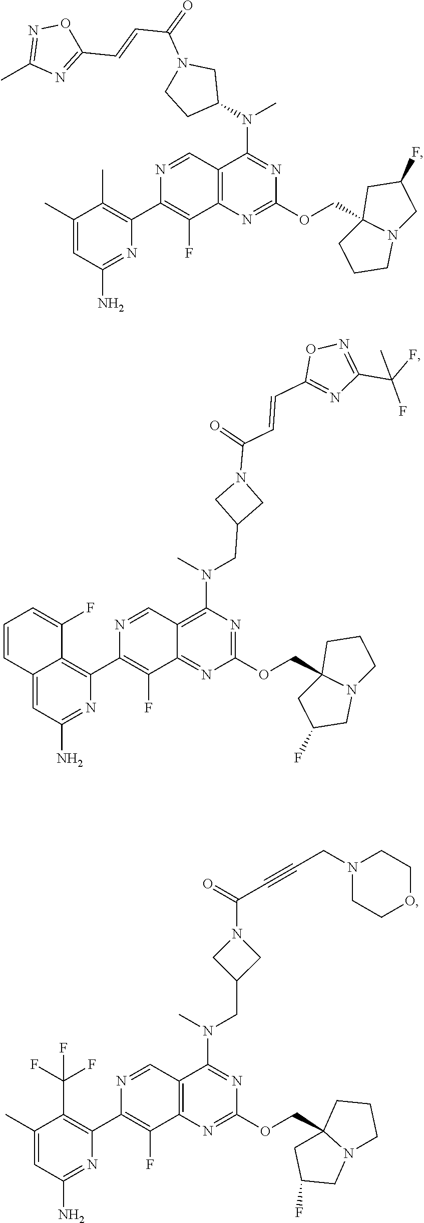 [SMILES: Cc1noc(/C=C/C(=O)N2CC[C@@H](N(C)c3nc(OC[C@@]45CCCN4C[C@H](F)C5)nc4c(F)c(-c5nc(N)cc(C)c5C)ncc34)C2)n1 ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(F)c34)ncc12 ; Cc1cc(N)nc(-c2ncc3c(N(C)CC4CN(C(=O)C#CCN5CCOCC5)C4)nc(OC[C@@]45CCCN4C[C@H](F)C5)nc3c2F)c1C(F)(F)F] 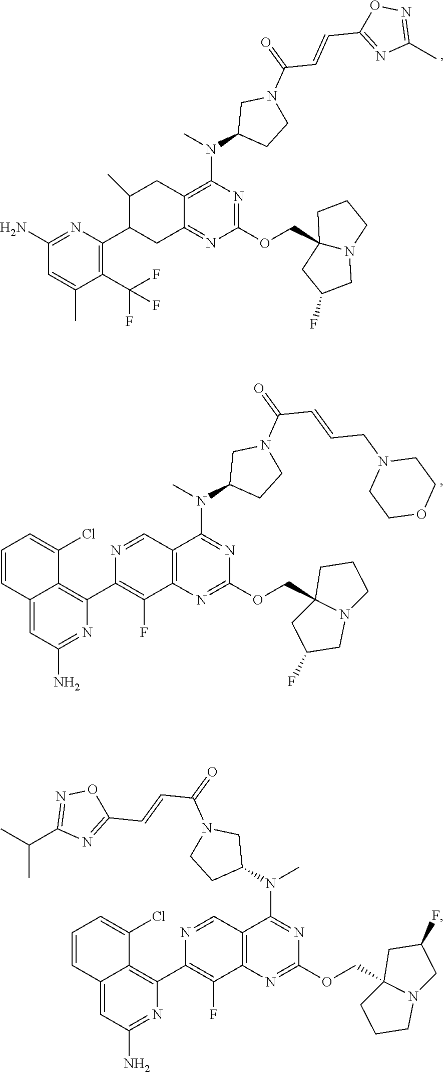 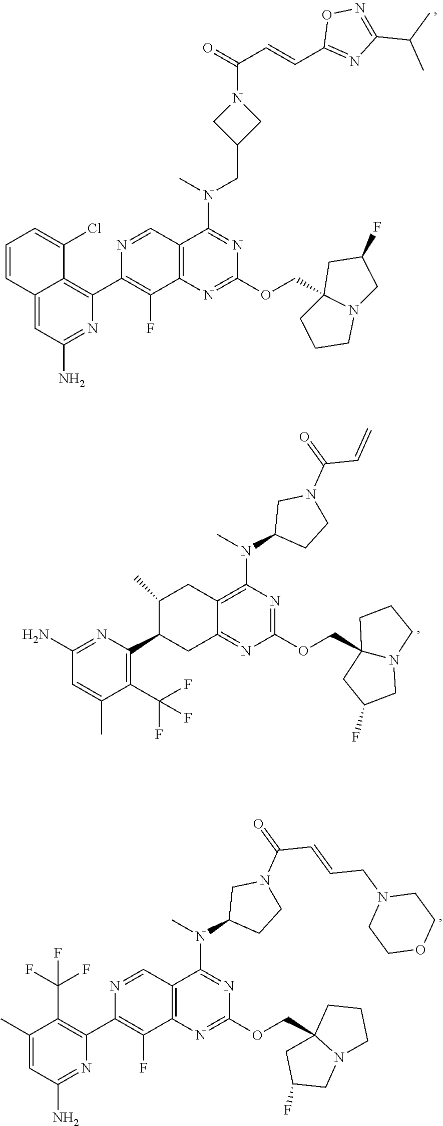 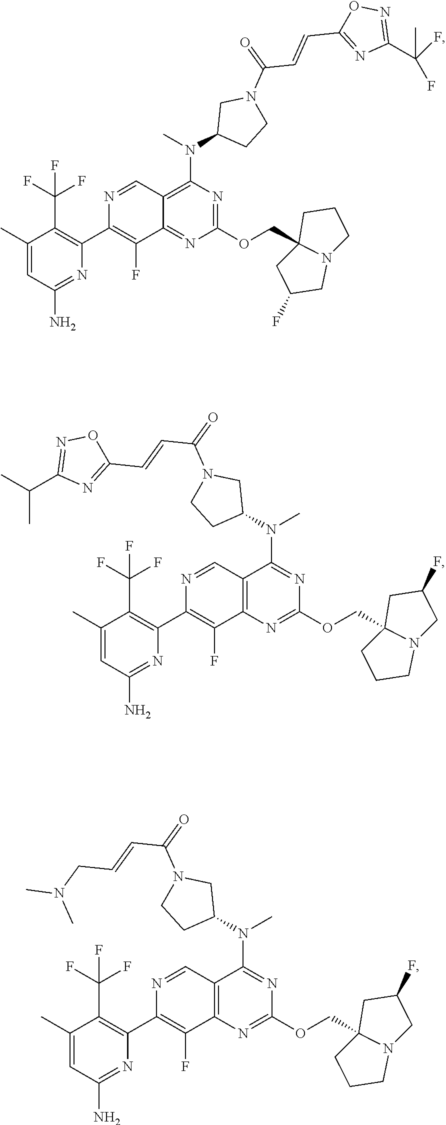 [SMILES: Cc1cc(N)nc(-c2ncc3c(N(C)[C@@H]4CCN(C(=O)/C=C/c5nc(C(C)(F)F)no5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C(F)(F)F ; Cc1cc(N)nc(-c2ncc3c(N(C)[C@@H]4CCN(C(=O)/C=C/CN(C)C)C4)nc(OC[C@@]45CCCN4C[C@H](F)C5)nc3c2F)c1C(F)(F)F] 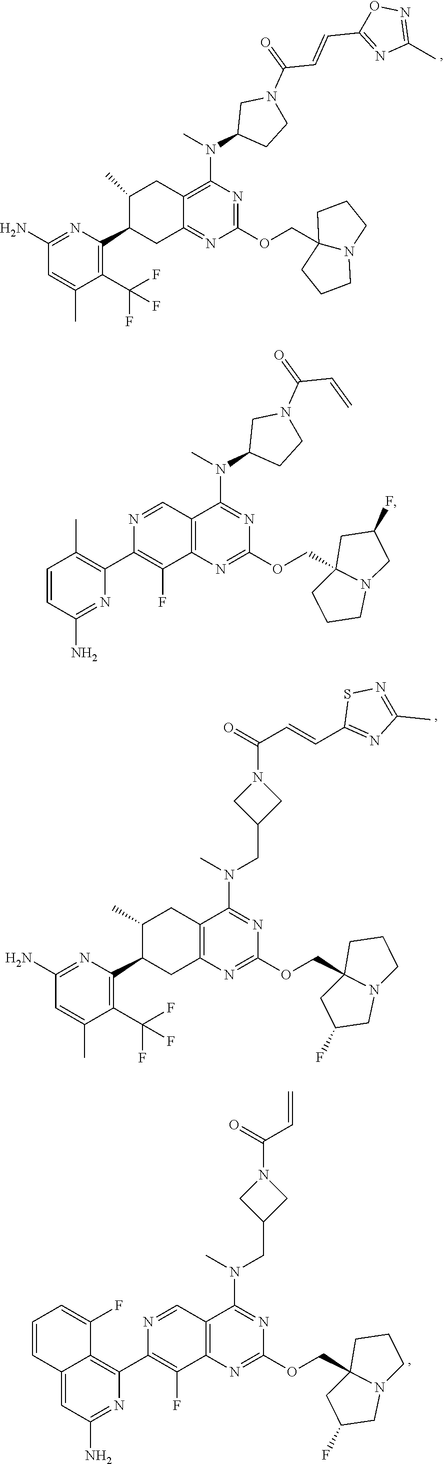 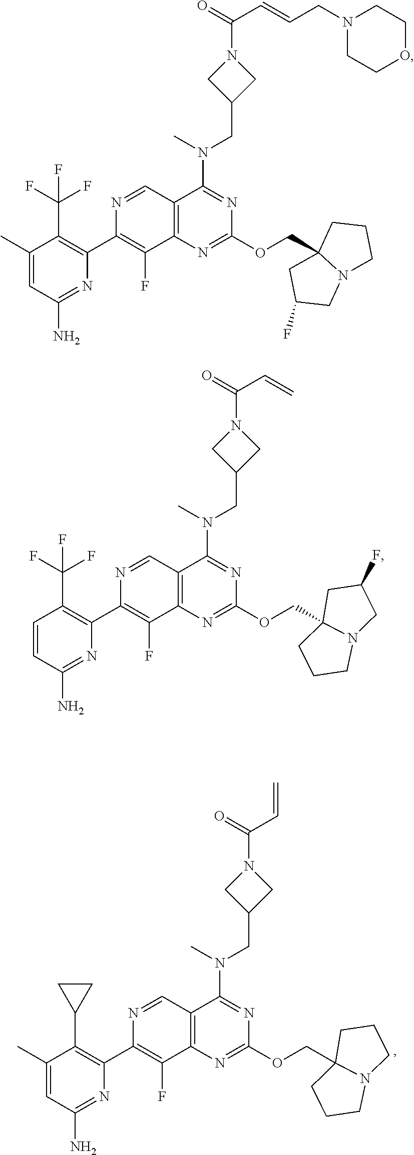 [SMILES: Cc1cc(N)nc(-c2ncc3c(N(C)CC4CN(C(=O)/C=C/CN5CCOCC5)C4)nc(OC[C@@]45CCCN4C[C@H](F)C5)nc3c2F)c1C(F)(F)F ; C=CC(=O)N1CC(CN(C)c2nc(OC[C@@]34CCCN3C[C@H](F)C4)nc3c(F)c(-c4nc(N)ccc4C(F)(F)F)ncc23)C1 ; C=CC(=O)N1CC(CN(C)c2nc(OCC34CCCN3CCC4)nc3c(F)c(-c4nc(N)cc(C)c4C4CC4)ncc23)C1] 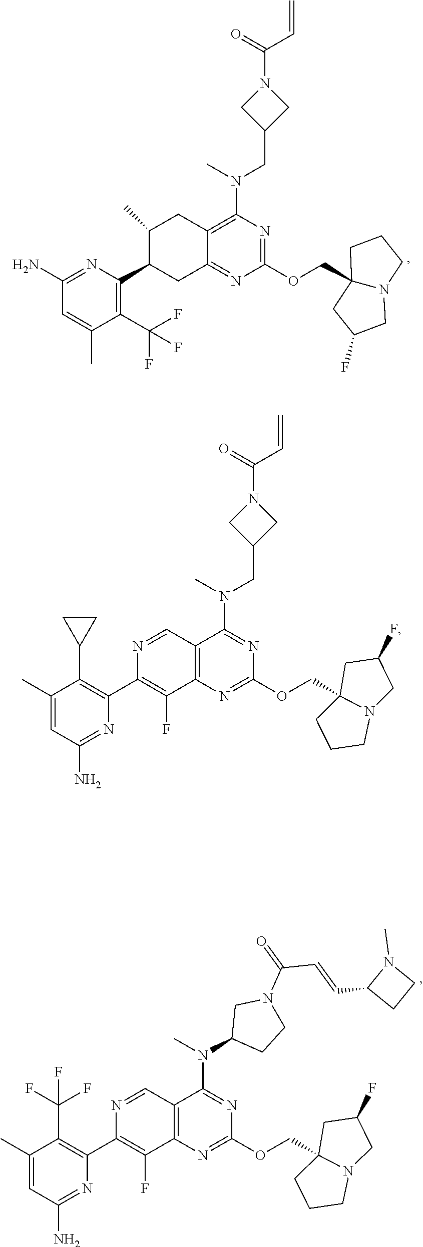 [SMILES: C=CC(=O)N1CC(CN(C)c2nc(OC[C@@]34CCCN3C[C@H](F)C4)nc3c2C[C@@H](C)[C@H](c2nc(N)cc(C)c2C(F)(F)F)C3)C1 ; C=CC(=O)N1CC(CN(C)c2nc(OC[C@@]34CCCN3C[C@H](F)C4)nc3c(F)c(-c4nc(N)cc(C)c4C4CC4)ncc23)C1 ; Cc1cc(N)nc(-c2ncc3c(N(C)[C@@H]4CCN(C(=O)/C=C/[C@H]5CCN5C)C4)nc(OC[C@@]45CCCN4C[C@H](F)C5)nc3c2F)c1C(F)(F)F] 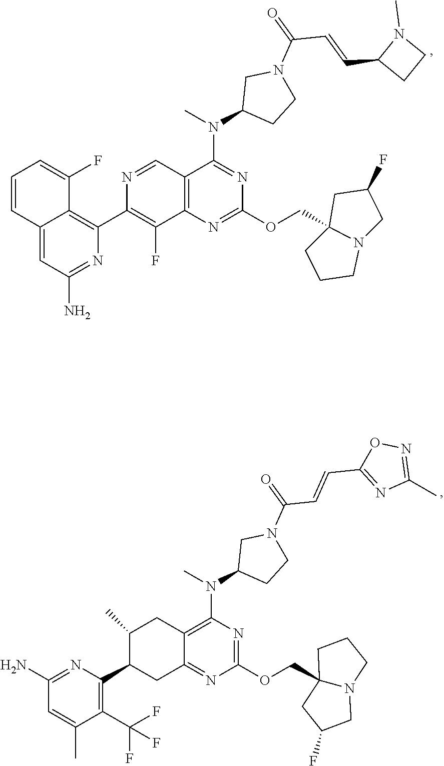 [SMILES: CN1CC[C@H]1/C=C/C(=O)N1CC[C@@H](N(C)c2nc(OC[C@@]34CCCN3C[C@H](F)C4)nc3c(F)c(-c4nc(N)cc5cccc(F)c45)ncc23)C1 ; Cc1noc(/C=C/C(=O)N2CC[C@@H](N(C)c3nc(OC[C@@]45CCCN4C[C@H](F)C5)nc4c3C[C@@H](C)[C@H](c3nc(N)cc(C)c3C(F)(F)F)C4)C2)n1] 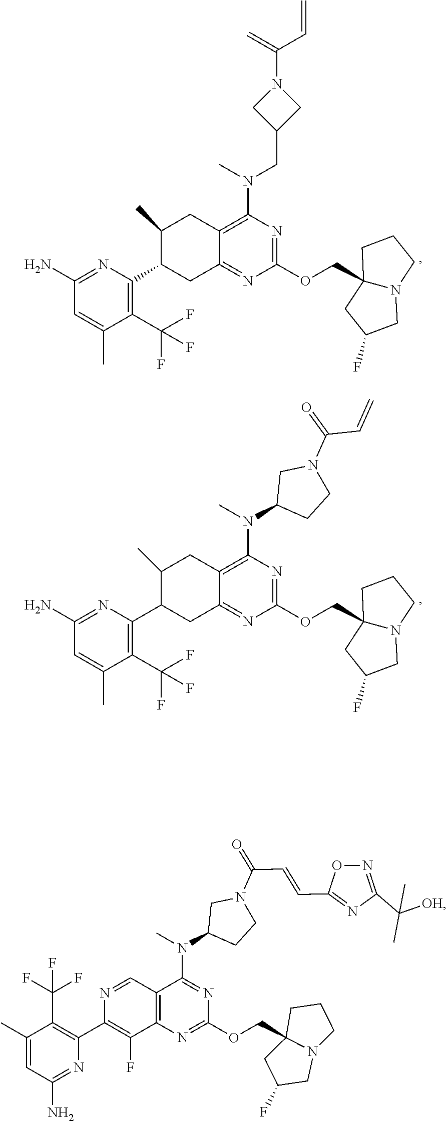 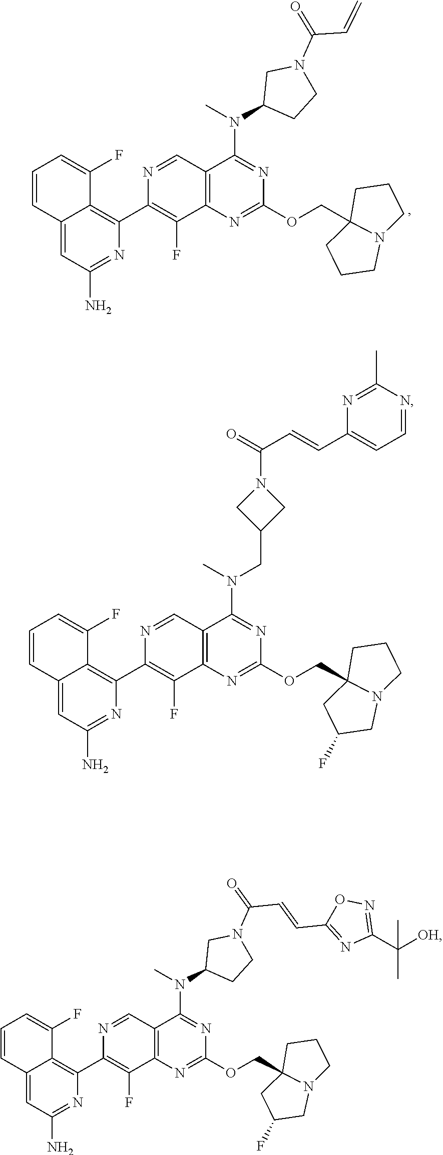 [SMILES: C=CC(=O)N1CC[C@@H](N(C)c2nc(OCC34CCCN3CCC4)nc3c(F)c(-c4nc(N)cc5cccc(F)c45)ncc23)C1 ; Cc1nccc(/C=C/C(=O)N2CC(CN(C)c3nc(OC[C@@]45CCCN4C[C@H](F)C5)nc4c(F)c(-c5nc(N)cc6cccc(F)c56)ncc34)C2)n1 ; CN(c1nc(OC[C@@]23CCCN2C[C@H](F)C3)nc2c(F)c(-c3nc(N)cc4cccc(F)c34)ncc12)[C@@H]1CCN(C(=O)/C=C/c2nc(C(C)(C)O)no2)C1] 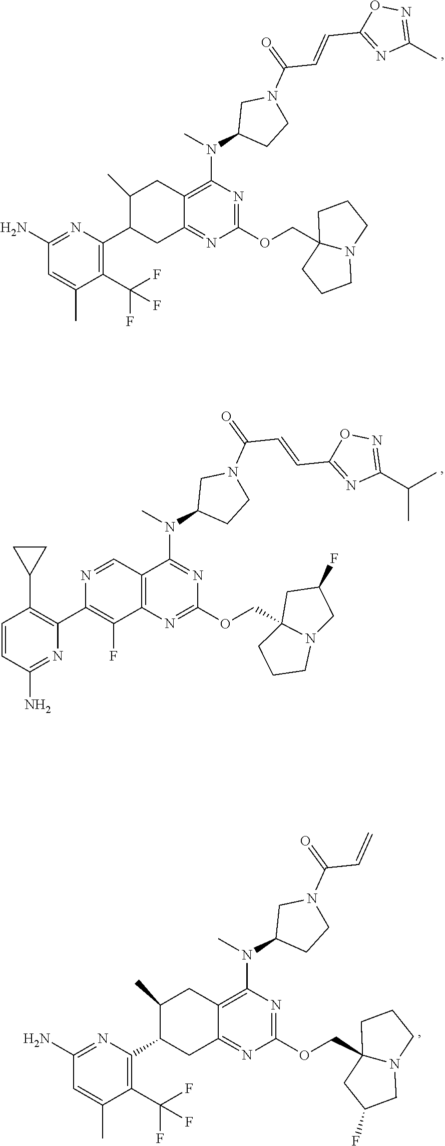 [SMILES: Cc1noc(/C=C/C(=O)N2CC[C@@H](N(C)c3nc(OCC45CCCN4CCC5)nc4c3CC(C)C(c3nc(N)cc(C)c3C(F)(F)F)C4)C2)n1 ; CC(C)c1noc(/C=C/C(=O)N2CC[C@@H](N(C)c3nc(OC[C@@]45CCCN4C[C@H](F)C5)nc4c(F)c(-c5nc(N)ccc5C5CC5)ncc34)C2)n1 ; C=CC(=O)N1CC[C@@H](N(C)c2nc(OC[C@@]34CCCN3C[C@H](F)C4)nc3c2C[C@H](C)[C@@H](c2nc(N)cc(C)c2C(F)(F)F)C3)C1] 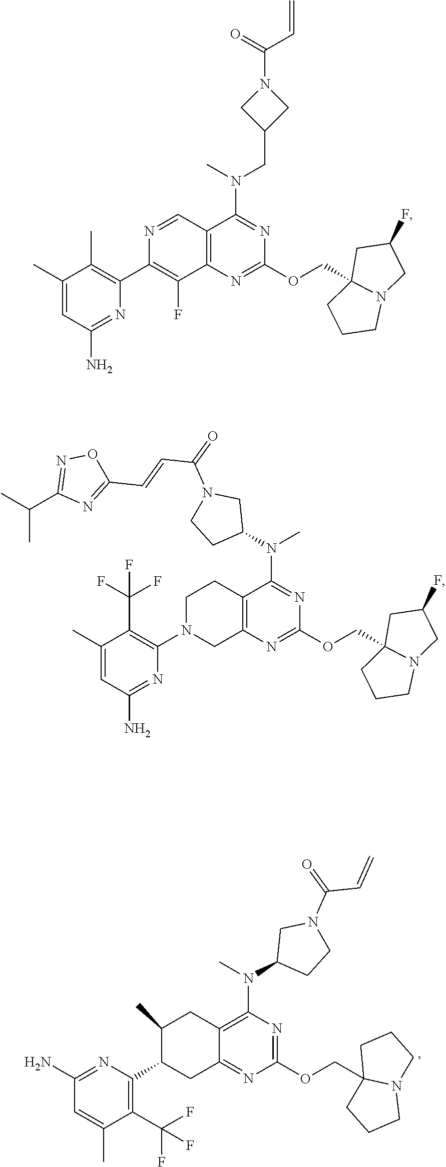 [SMILES: C=CC(=O)N1CC(CN(C)c2nc(OC[C@@]34CCCN3C[C@H](F)C4)nc3c(F)c(-c4nc(N)cc(C)c4C)ncc23)C1 ; Cc1cc(N)nc(N2CCc3c(nc(OC[C@@]45CCCN4C[C@H](F)C5)nc3N(C)[C@@H]3CCN(C(=O)/C=C/c4nc(C(C)C)no4)C3)C2)c1C(F)(F)F ; C=CC(=O)N1CC[C@@H](N(C)c2nc(OCC34CCCN3CCC4)nc3c2C[C@H](C)[C@@H](c2nc(N)cc(C)c2C(F)(F)F)C3)C1] 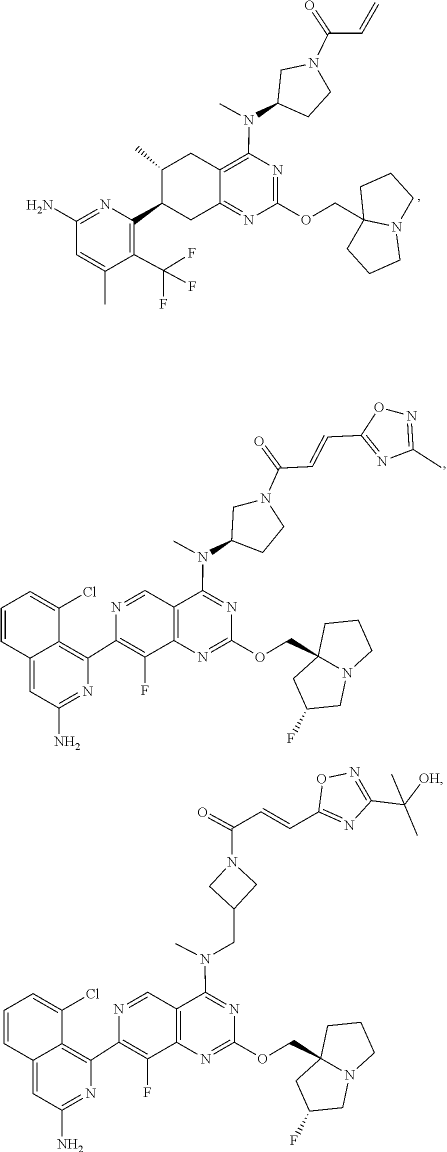 [SMILES: C=CC(=O)N1CC[C@@H](N(C)c2nc(OCC34CCCN3CCC4)nc3c2C[C@@H](C)[C@H](c2nc(N)cc(C)c2C(F)(F)F)C3)C1 ; Cc1noc(/C=C/C(=O)N2CC[C@@H](N(C)c3nc(OC[C@@]45CCCN4C[C@H](F)C5)nc4c(F)c(-c5nc(N)cc6cccc(Cl)c56)ncc34)C2)n1 ; CN(CC1CN(C(=O)/C=C/c2nc(C(C)(C)O)no2)C1)c1nc(OC[C@@]23CCCN2C[C@H](F)C3)nc2c(F)c(-c3nc(N)cc4cccc(Cl)c34)ncc12] 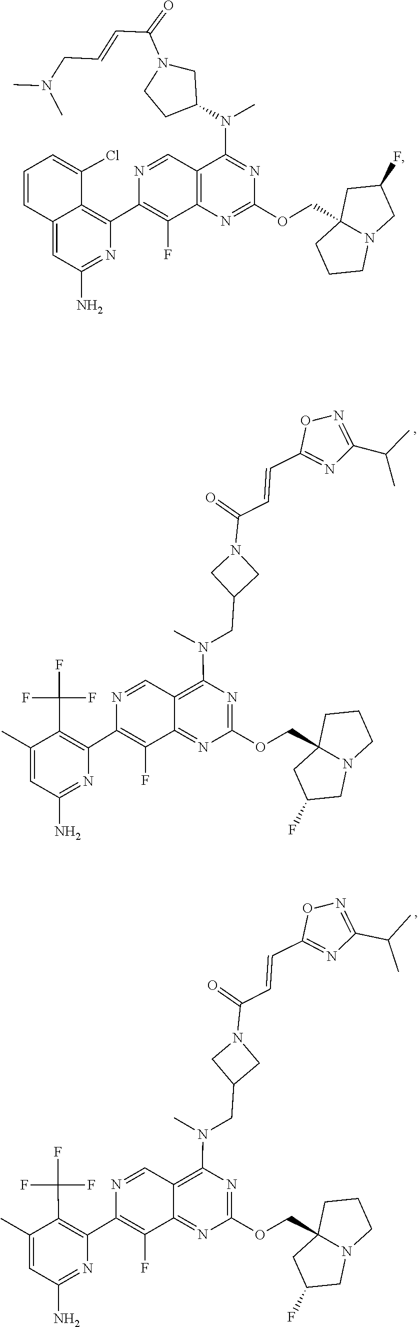 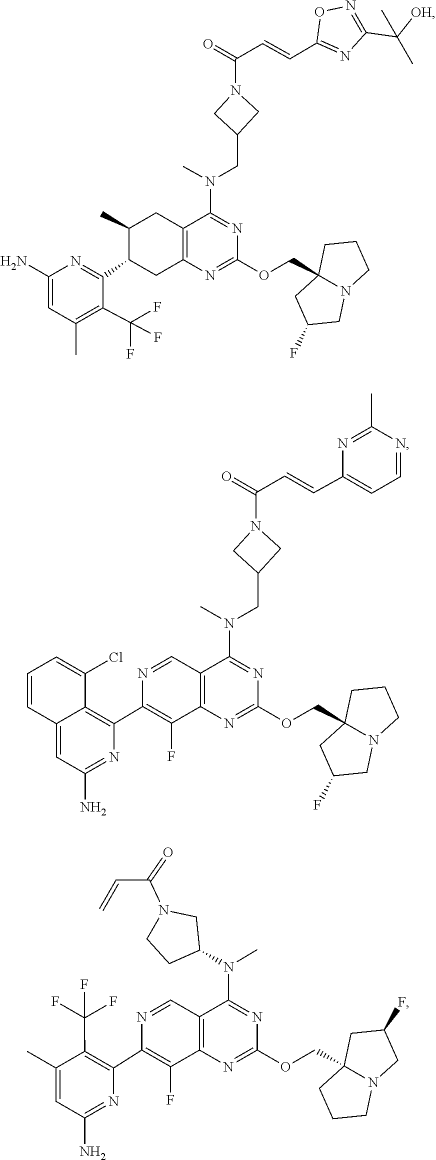 [SMILES: Cc1cc(N)nc([C@H]2Cc3nc(OC[C@@]45CCCN4C[C@H](F)C5)nc(N(C)CC4CN(C(=O)/C=C/c5nc(C(C)(C)O)no5)C4)c3C[C@@H]2C)c1C(F)(F)F ; Cc1nccc(/C=C/C(=O)N2CC(CN(C)c3nc(OC[C@@]45CCCN4C[C@H](F)C5)nc4c(F)c(-c5nc(N)cc6cccc(Cl)c56)ncc34)C2)n1 ; C=CC(=O)N1CC[C@@H](N(C)c2nc(OC[C@@]34CCCN3C[C@H](F)C4)nc3c(F)c(-c4nc(N)cc(C)c4C(F)(F)F)ncc23)C1] 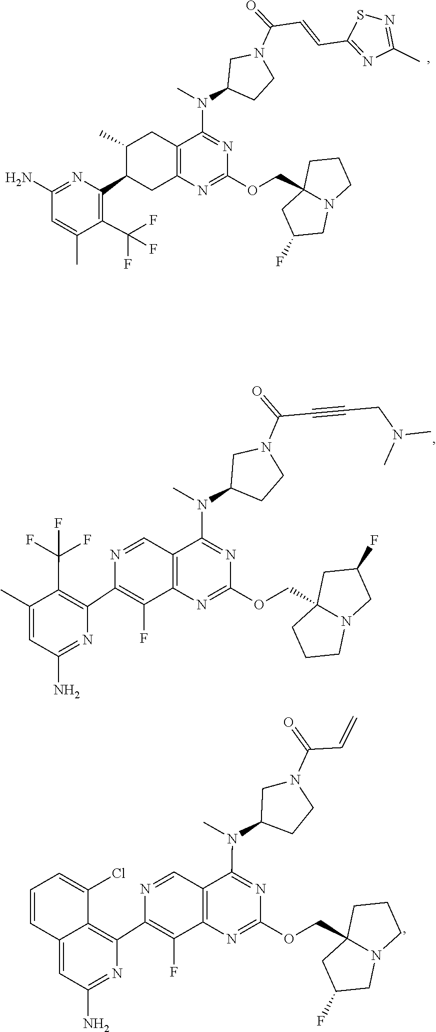 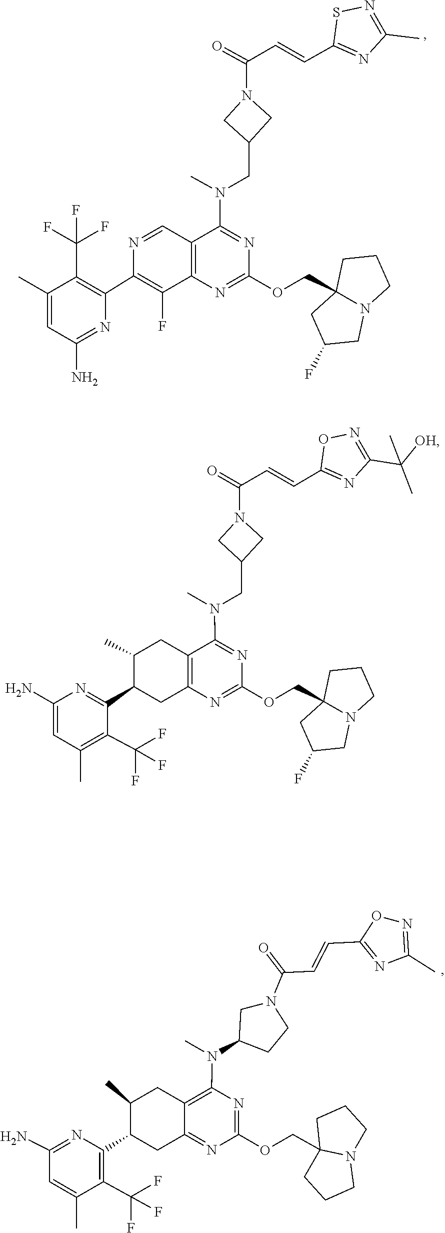 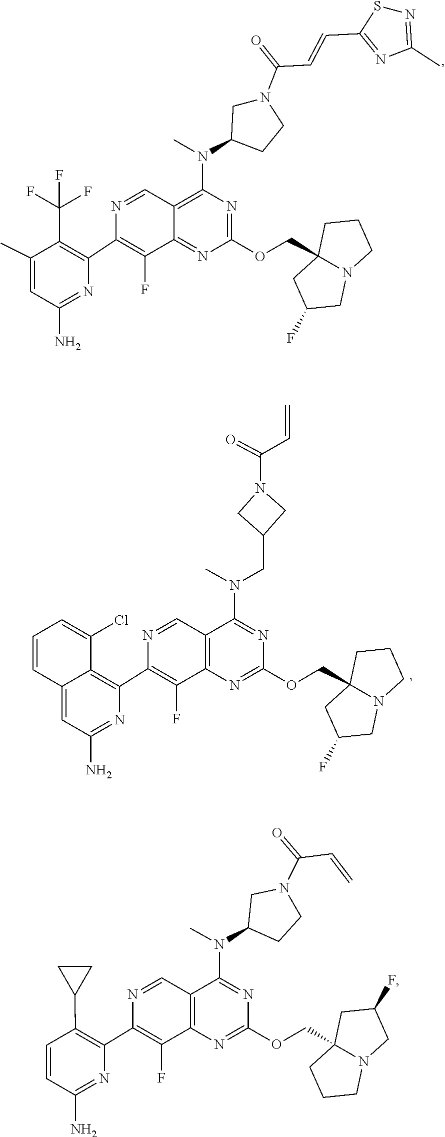 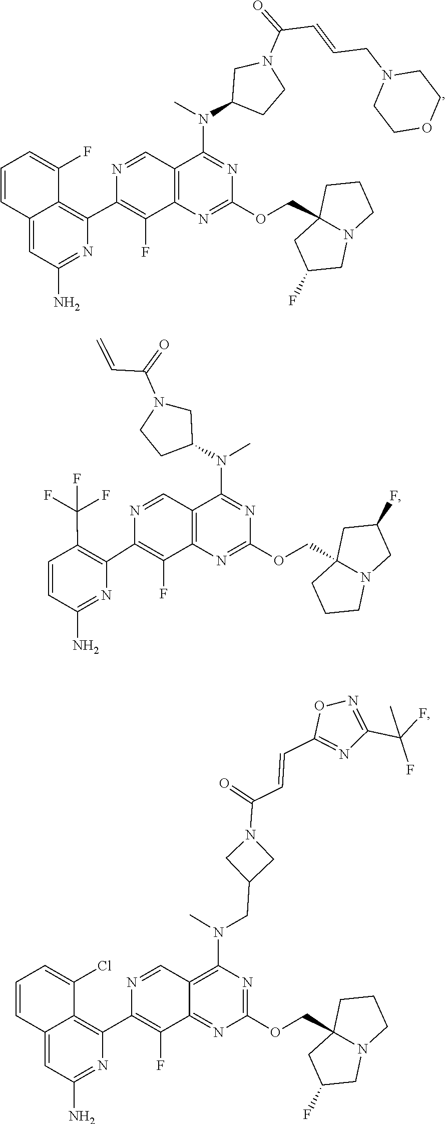 [SMILES: CN(c1nc(OC[C@@]23CCCN2C[C@H](F)C3)nc2c(F)c(-c3nc(N)cc4cccc(F)c34)ncc12)[C@@H]1CCN(C(=O)/C=C/CN2CCOCC2)C1 ; C=CC(=O)N1CC[C@@H](N(C)c2nc(OC[C@@]34CCCN3C[C@H](F)C4)nc3c(F)c(-c4nc(N)ccc4C(F)(F)F)ncc23)C1 ; CN(CC1CN(C(=O)/C=C/c2nc(C(C)(F)F)no2)C1)c1nc(OC[C@@]23CCCN2C[C@H](F)C3)nc2c(F)c(-c3nc(N)cc4cccc(Cl)c34)ncc12] 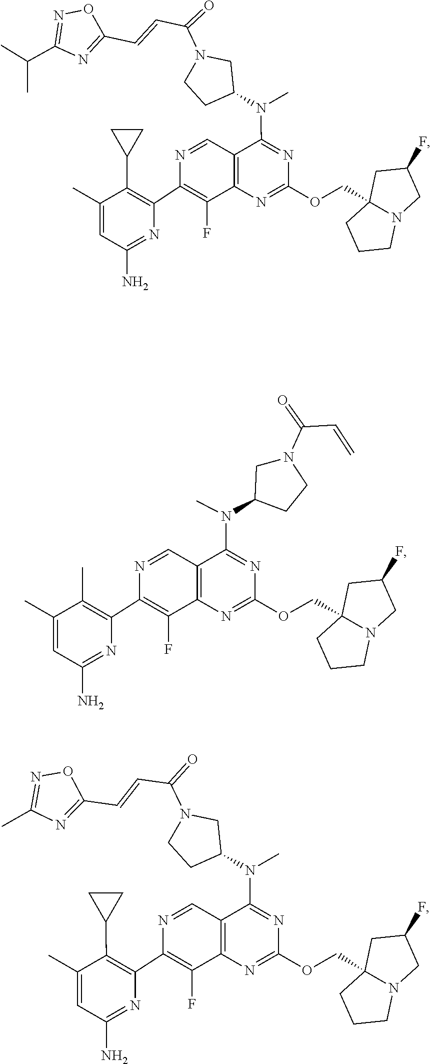 [SMILES: Cc1cc(N)nc(-c2ncc3c(N(C)[C@@H]4CCN(C(=O)/C=C/c5nc(C(C)C)no5)C4)nc(OC[C@@]45CCCN4C[C@H](F)C5)nc3c2F)c1C1CC1 ; C=CC(=O)N1CC[C@@H](N(C)c2nc(OC[C@@]34CCCN3C[C@H](F)C4)nc3c(F)c(-c4nc(N)cc(C)c4C)ncc23)C1 ; Cc1noc(/C=C/C(=O)N2CC[C@@H](N(C)c3nc(OC[C@@]45CCCN4C[C@H](F)C5)nc4c(F)c(-c5nc(N)cc(C)c5C5CC5)ncc34)C2)n1] 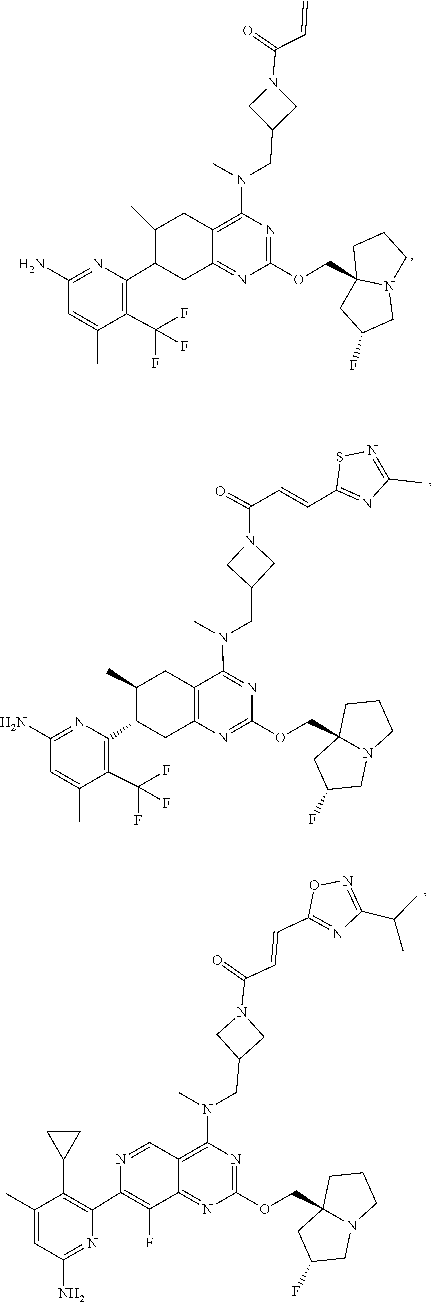 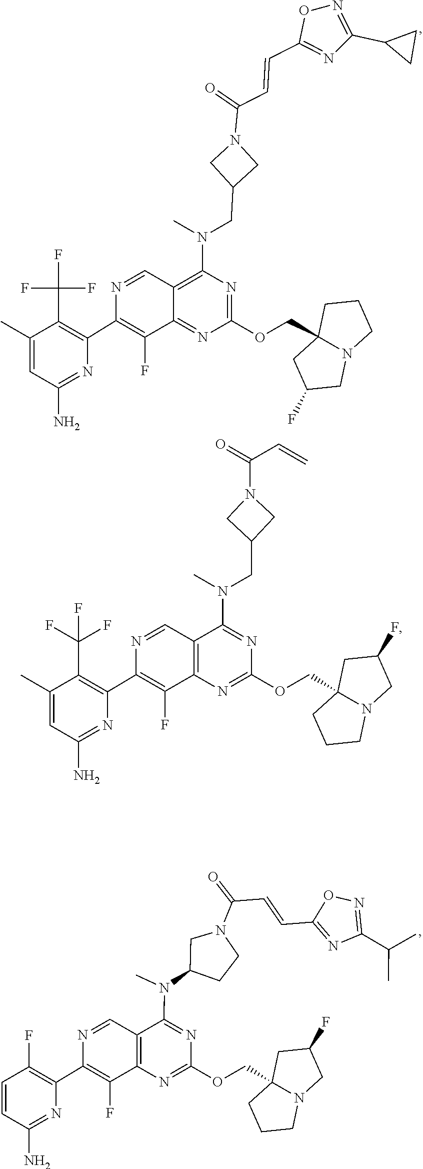 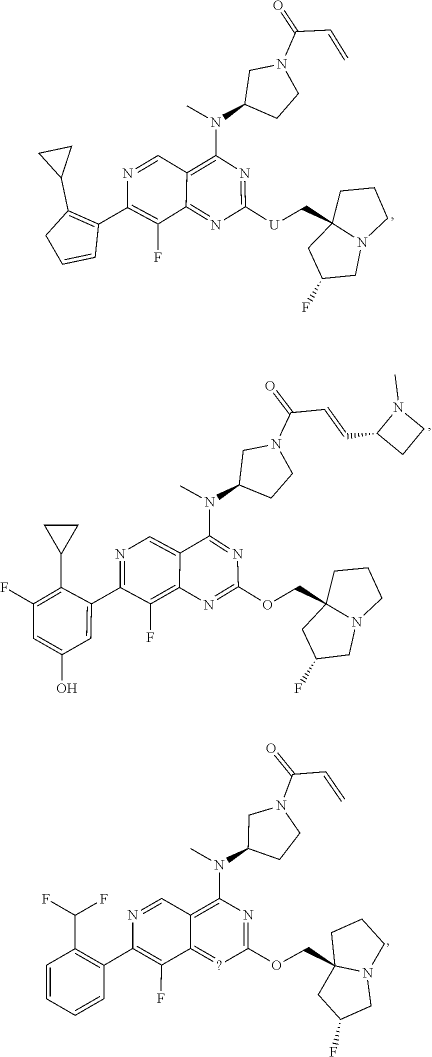 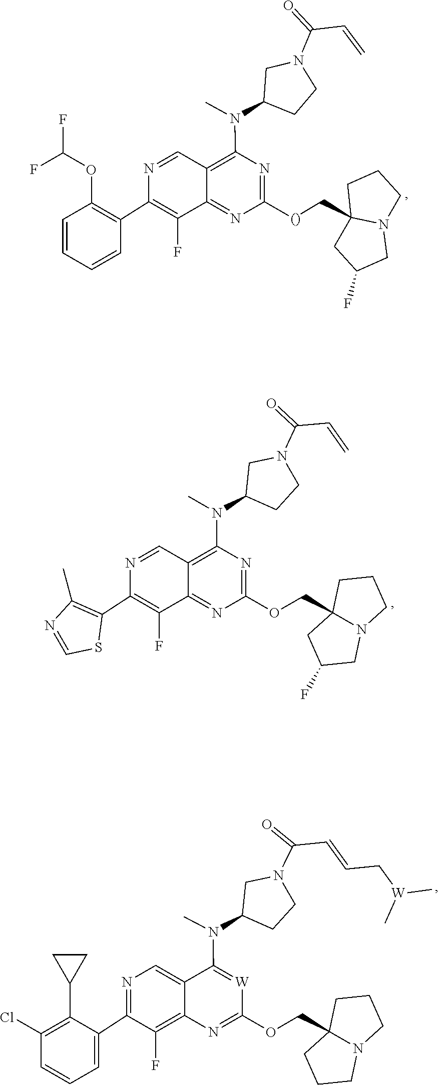 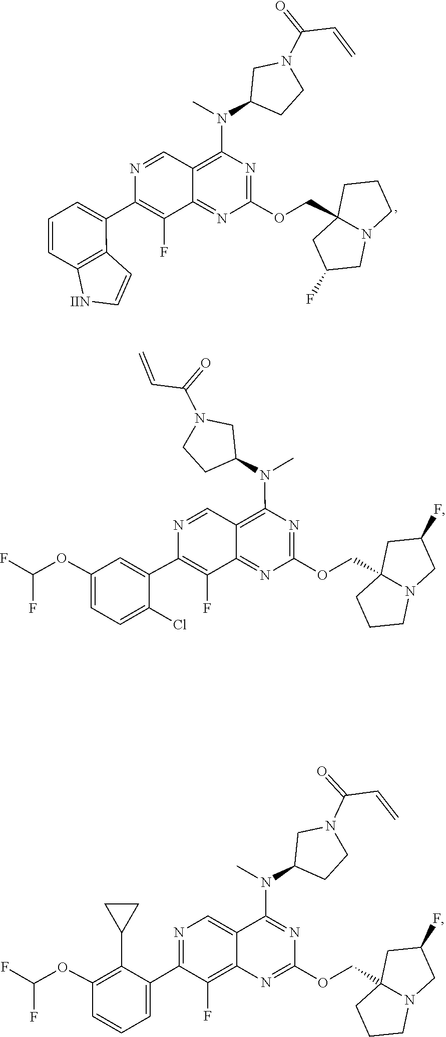 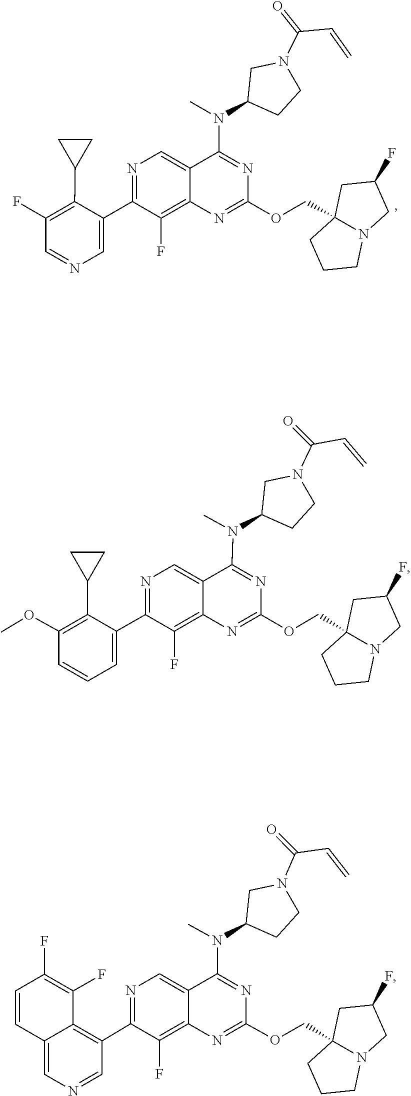 [SMILES: C=CC(=O)N1CC[C@@H](N(C)c2nc(OC[C@@]34CCCN3C[C@H](F)C4)nc3c(F)c(-c4cncc(F)c4C4CC4)ncc23)C1 ; C=CC(=O)N1CC[C@@H](N(C)c2nc(OC[C@@]34CCCN3C[C@H](F)C4)nc3c(F)c(-c4cccc(OC)c4C4CC4)ncc23)C1 ; C=CC(=O)N1CC[C@@H](N(C)c2nc(OC[C@@]34CCCN3C[C@H](F)C4)nc3c(F)c(-c4cncc5ccc(F)c(F)c45)ncc23)C1] 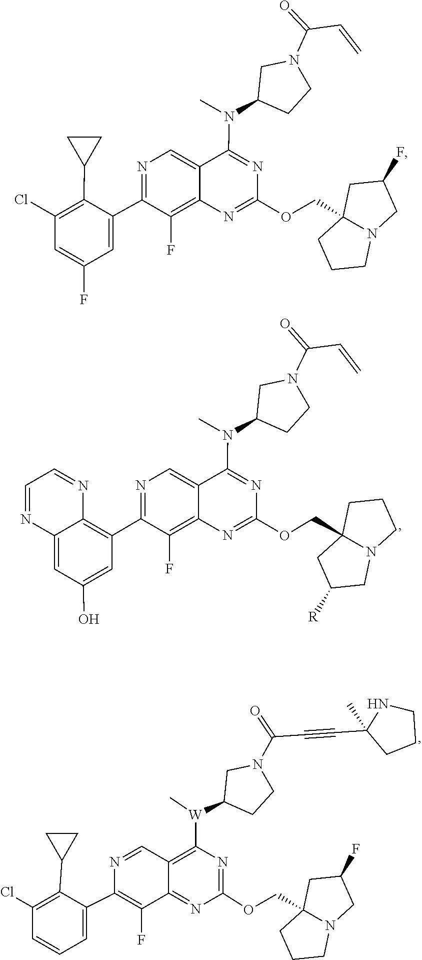 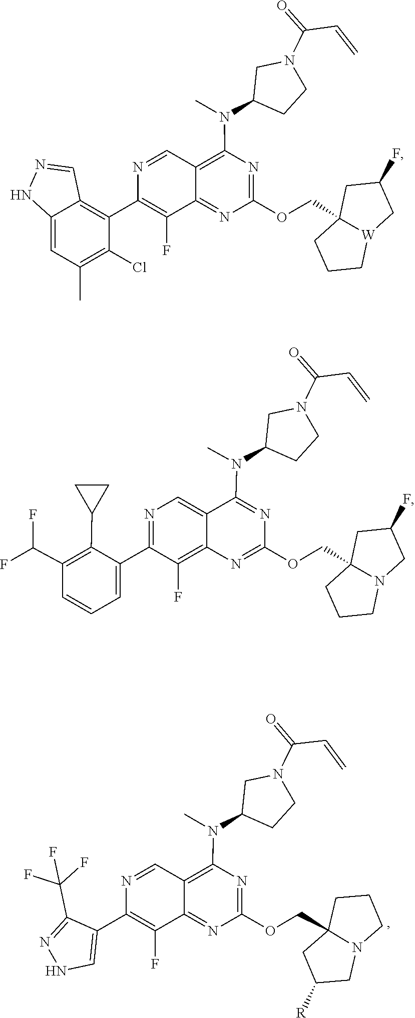 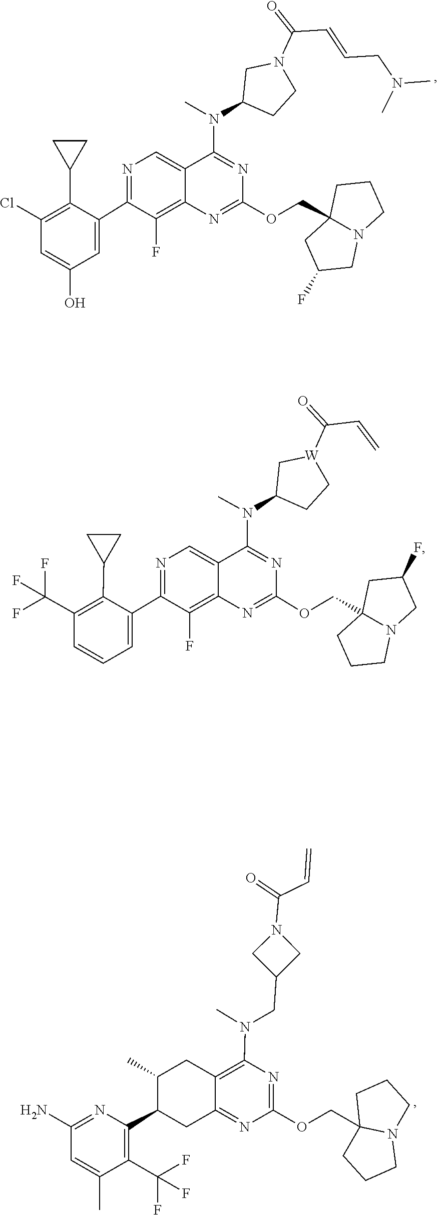 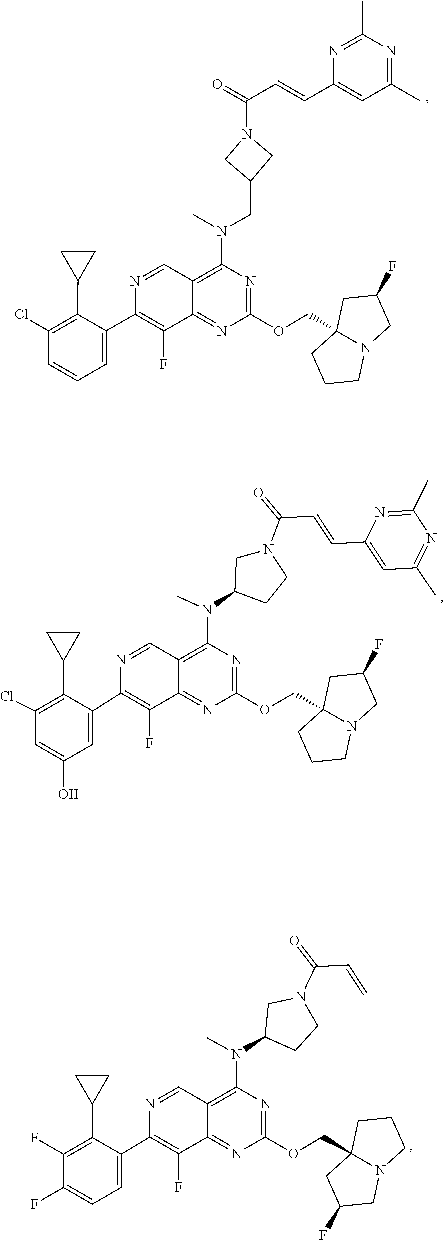 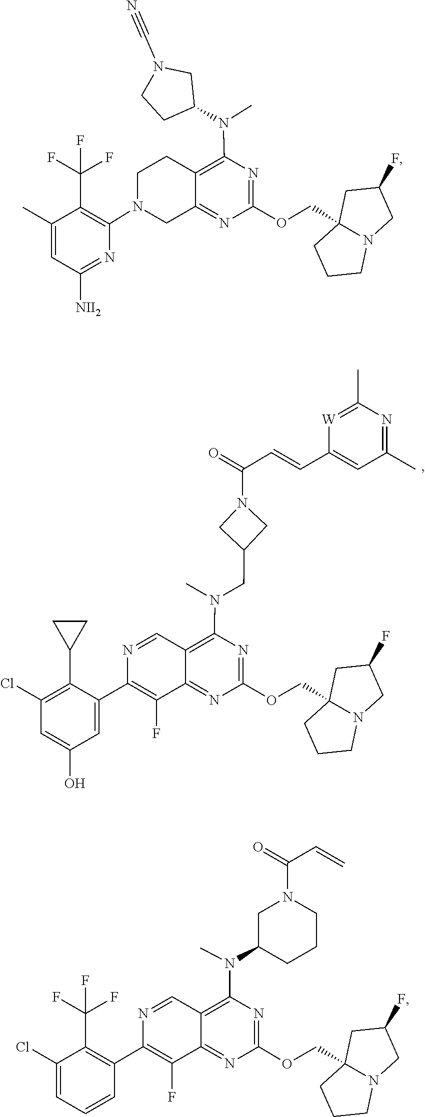 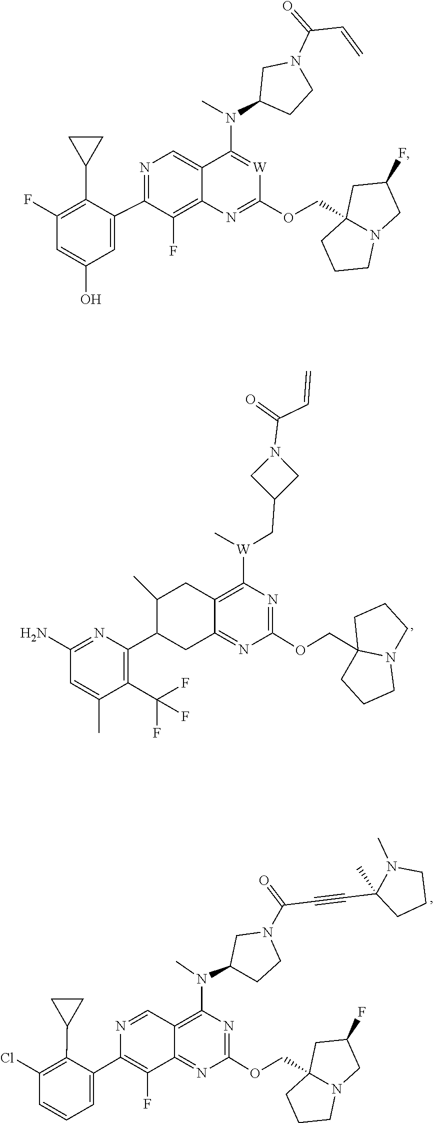 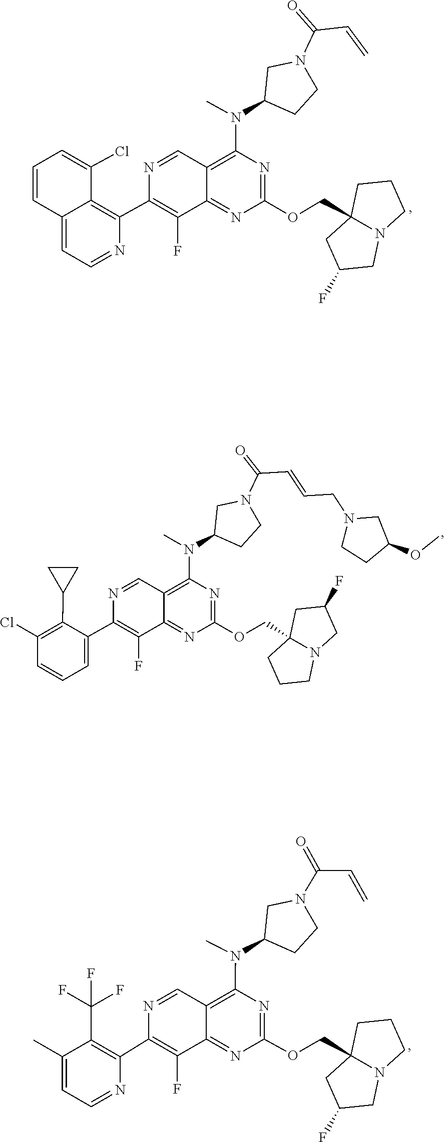 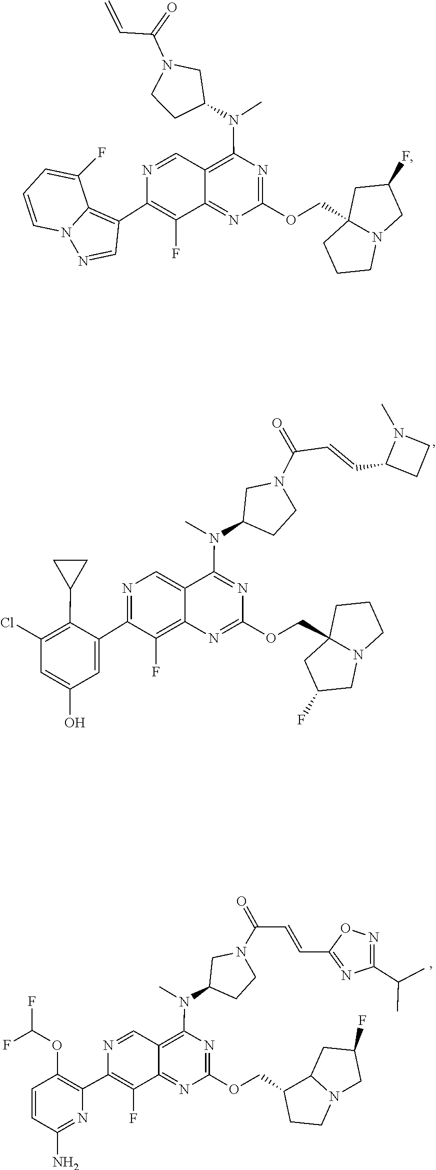 [SMILES: C=CC(=O)N1CC[C@@H](N(C)c2nc(OC[C@@]34CCCN3C[C@H](F)C4)nc3c(F)c(-c4cnn5cccc(F)c45)ncc23)C1 ; CN1CC[C@@H]1/C=C/C(=O)N1CC[C@@H](N(C)c2nc(OC[C@@]34CCCN3C[C@H](F)C4)nc3c(F)c(-c4cc(O)cc(Cl)c4C4CC4)ncc23)C1 ; CC(C)c1noc(/C=C/C(=O)N2CC[C@@H](N(C)c3nc(OC[C@H]4CCN5C[C@H](F)CC45)nc4c(F)c(-c5nc(N)ccc5OC(F)F)ncc34)C2)n1] 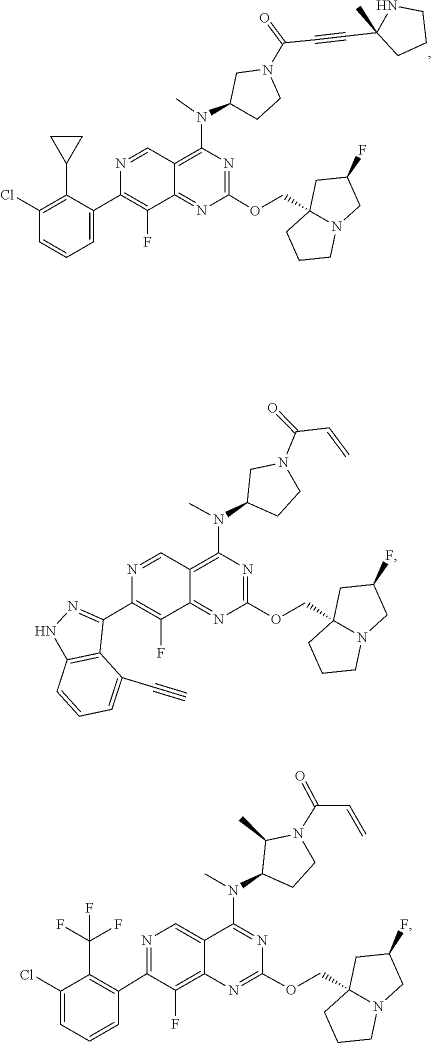 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#C[C@@]2(C)CCCN2)C1 ; C#Cc1cccc2[nH]nc(-c3ncc4c(N(C)[C@@H]5CCN(C(=O)C=C)C5)nc(OC[C@@]56CCCN5C[C@H](F)C6)nc4c3F)c12 ; C=CC(=O)N1CC[C@@H](N(C)c2nc(OC[C@@]34CCCN3C[C@H](F)C4)nc3c(F)c(-c4cccc(Cl)c4C(F)(F)F)ncc23)[C@H]1C] 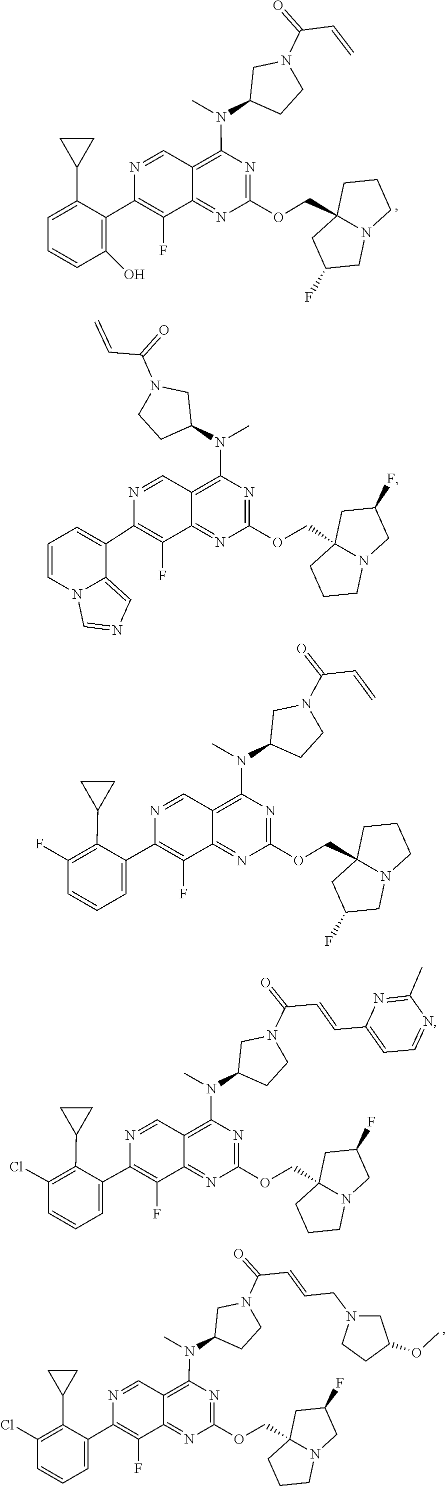 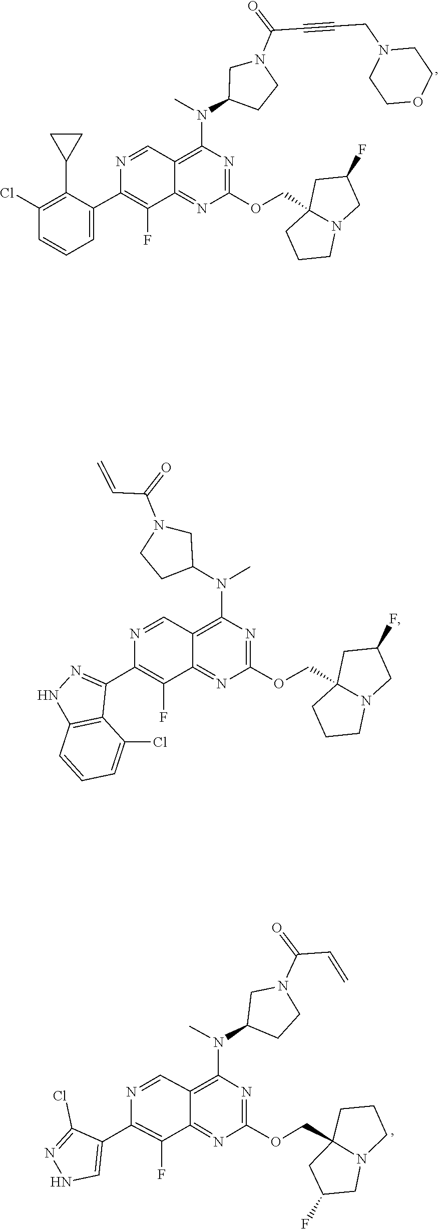 [SMILES: CN(c1nc(OC[C@@]23CCCN2C[C@H](F)C3)nc2c(F)c(-c3cccc(Cl)c3C3CC3)ncc12)[C@@H]1CCN(C(=O)C#CCN2CCOCC2)C1 ; C=CC(=O)N1CCC(N(C)c2nc(OC[C@@]34CCCN3C[C@H](F)C4)nc3c(F)c(-c4n[nH]c5cccc(Cl)c45)ncc23)C1 ; C=CC(=O)N1CC[C@@H](N(C)c2nc(OC[C@@]34CCCN3C[C@H](F)C4)nc3c(F)c(-c4c[nH]nc4Cl)ncc23)C1] 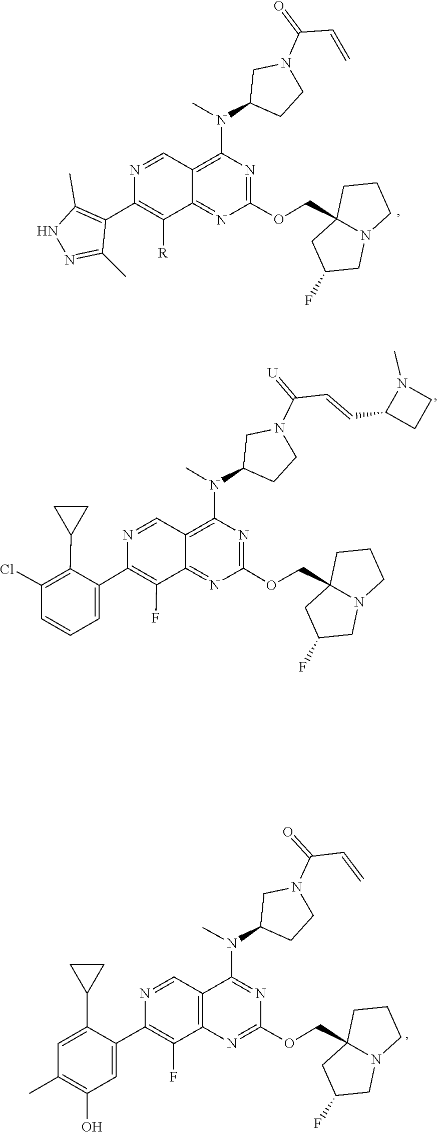 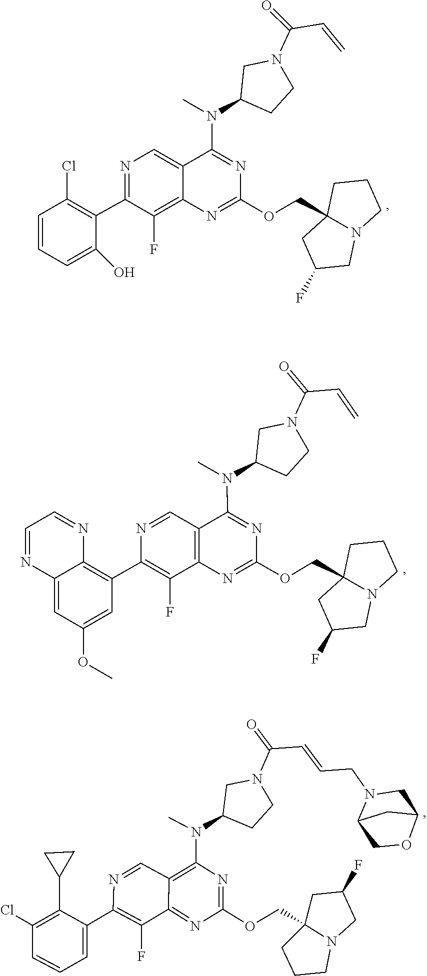 [SMILES: C=CC(=O)N1CC[C@@H](N(C)c2nc(OC[C@@]34CCCN3C[C@H](F)C4)nc3c(F)c(-c4c(O)cccc4Cl)ncc23)C1 ; C=CC(=O)N1CC[C@@H](N(C)c2nc(OC[C@@]34CCCN3C[C@@H](F)C4)nc3c(F)c(-c4cc(OC)cc5nccnc45)ncc23)C1 ; CN(c1nc(OC[C@@]23CCCN2C[C@H](F)C3)nc2c(F)c(-c3cccc(Cl)c3C3CC3)ncc12)[C@@H]1CCN(C(=O)/C=C/CN2C[C@@H]3C[C@H]2CO3)C1] 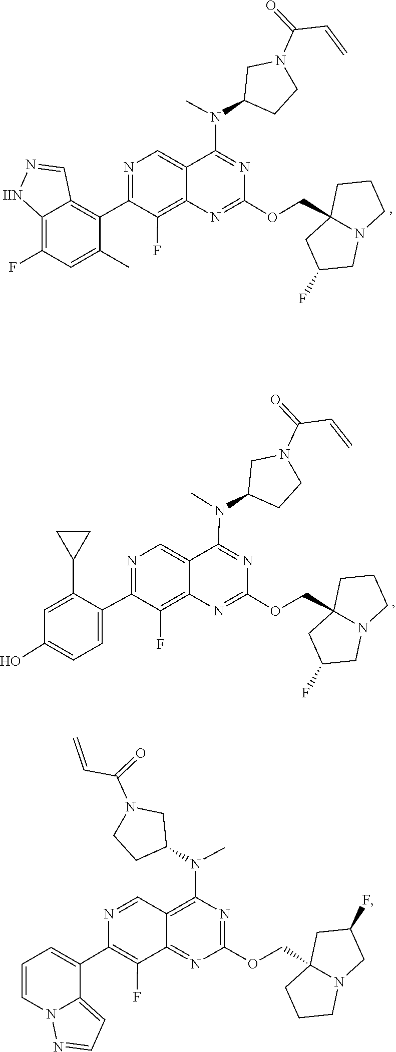 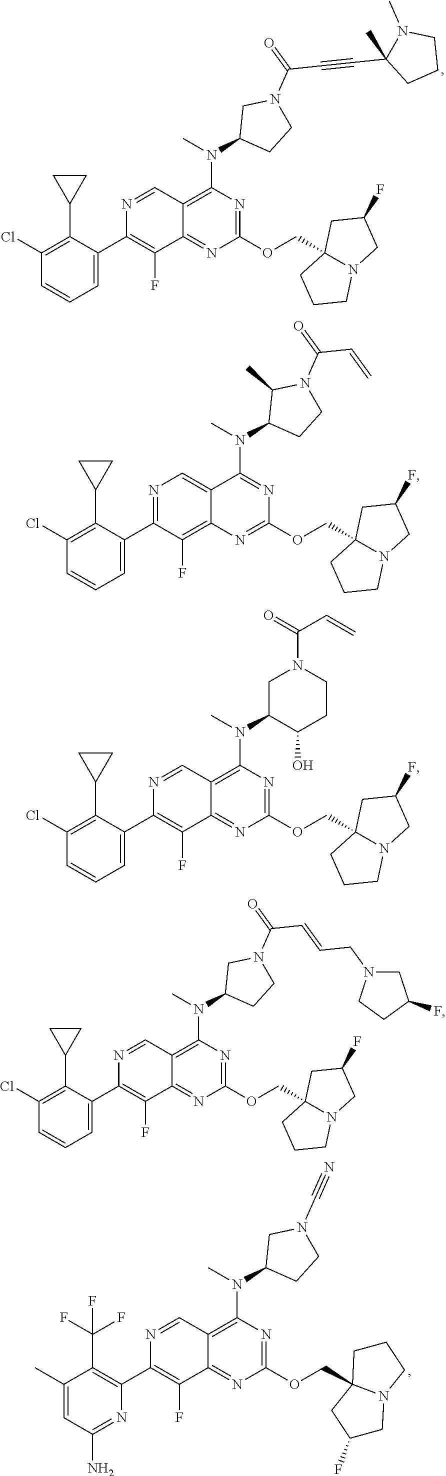 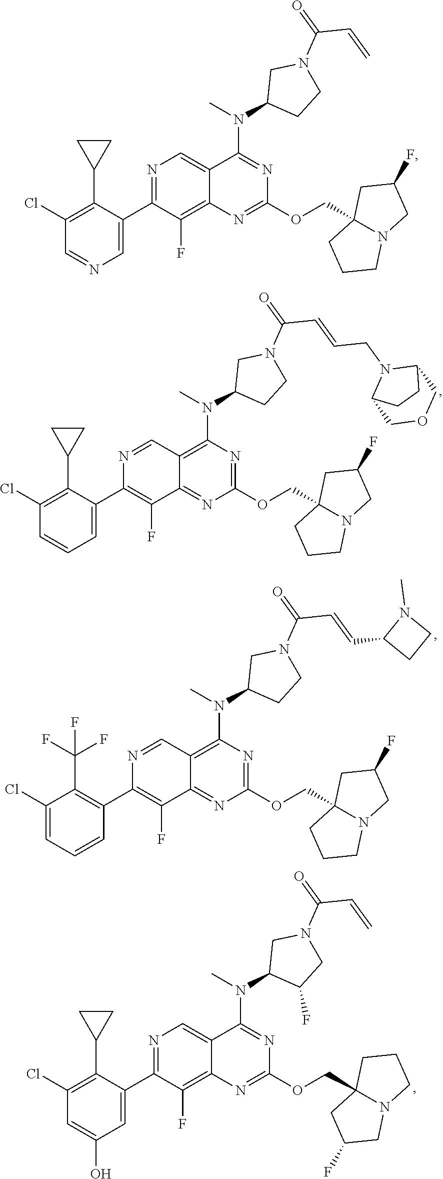 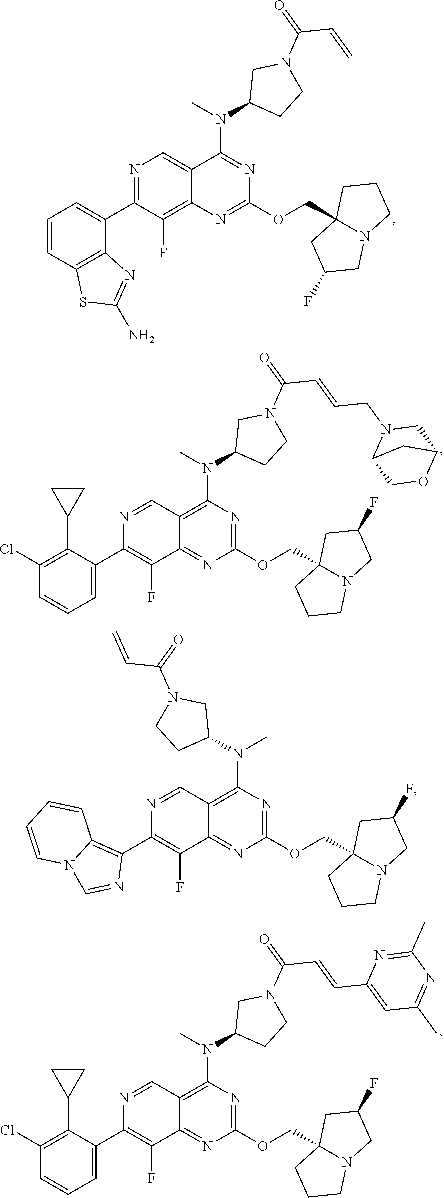 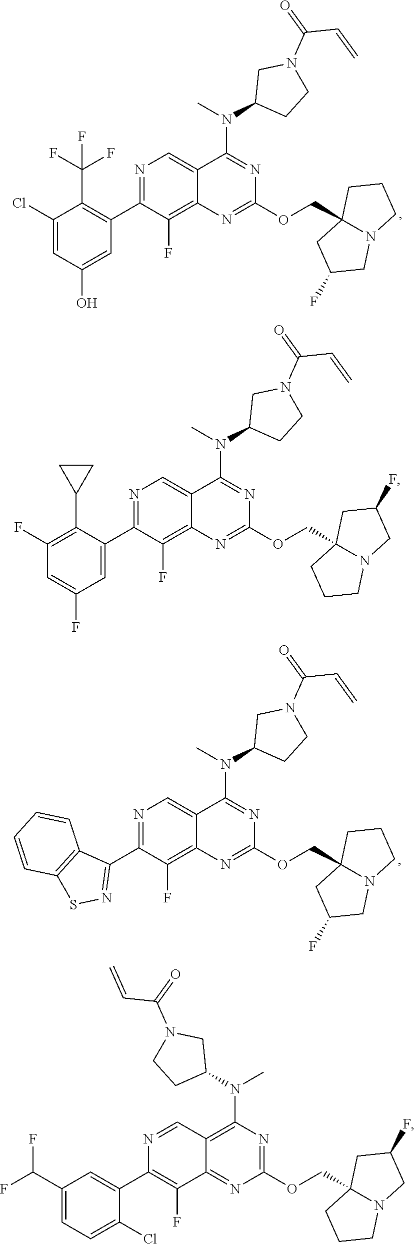 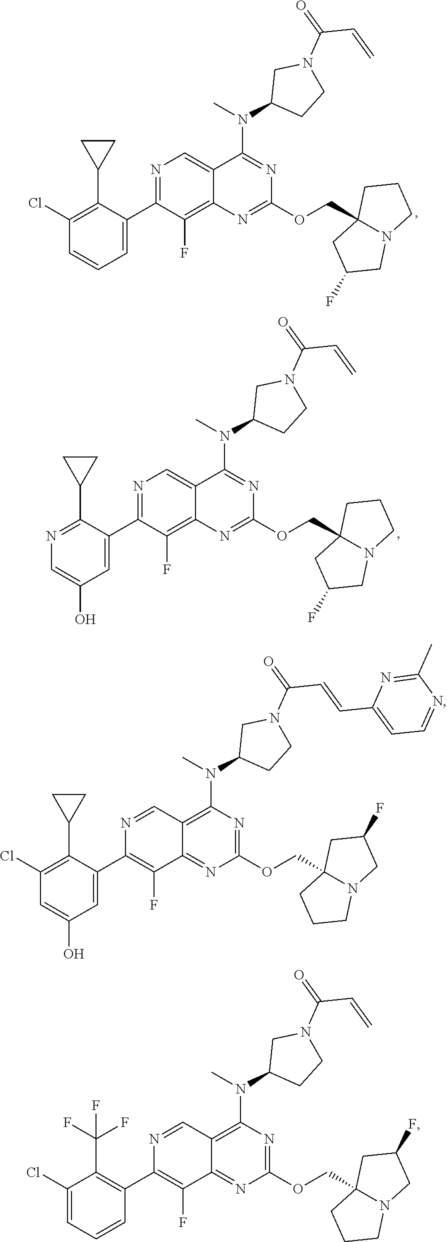 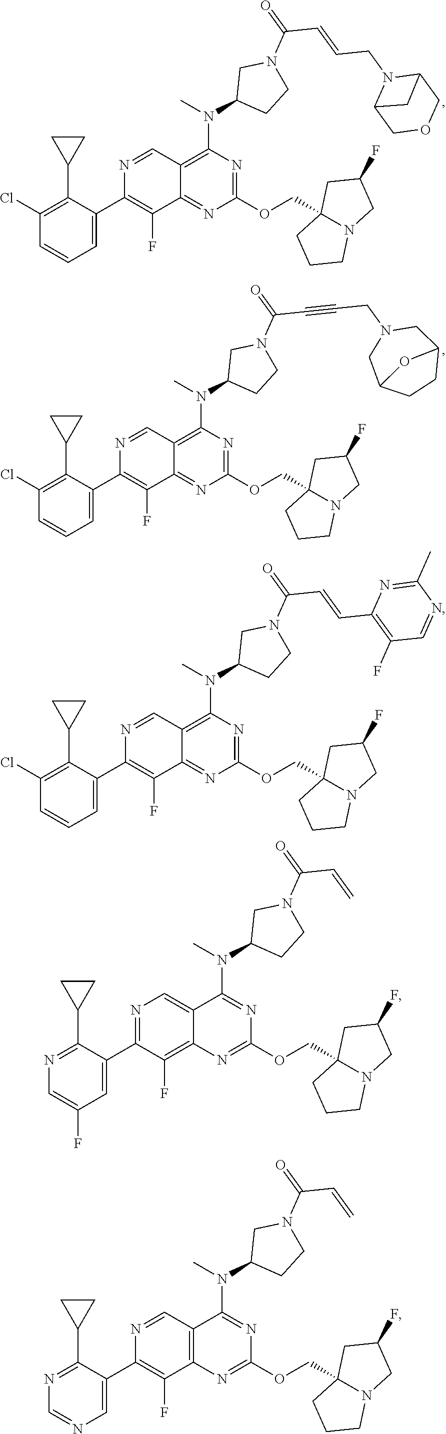 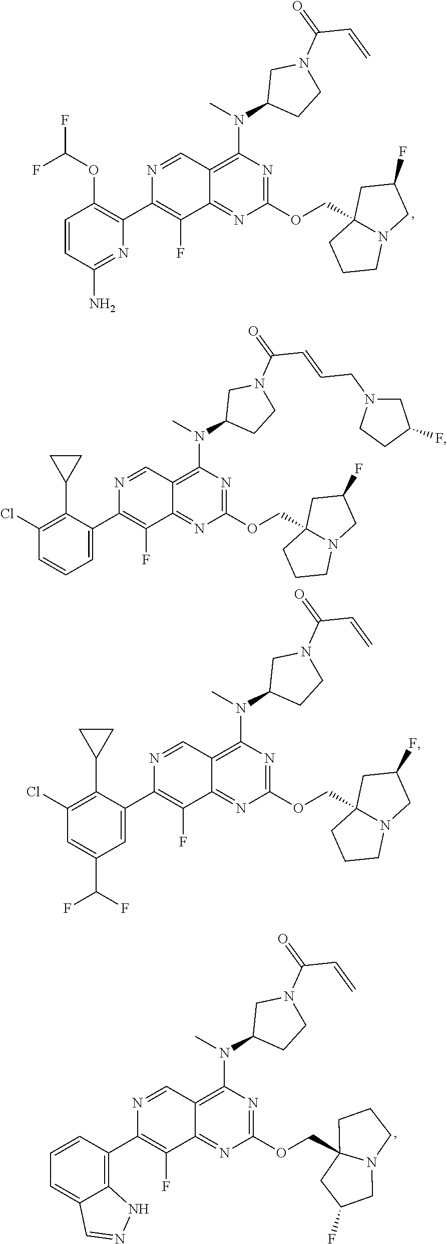
and all salts and isotopologues thereof.

118. The compound of any one of claims 1-117, wherein the compound is not a salt.

119. The compound of any one of claims 1-117, wherein the compound is a salt.

120. The compound of claim 119, wherein the salt is a formate salt.

121. The compound of claim 119, wherein the salt is a trifluoroacetate salt.

122. The compound of claim 119 wherein the salt is a pharmaceutically acceptable salt.

123. A pharmaceutical formulation comprising the compound of any one of claims 1-122, wherein when the compound is a salt, the salt is a pharmaceutically acceptable salt, and a pharmaceutically acceptable carrier.

124. A compound of any one of claims 1-122 or the pharmaceutical formulation of claim 123 for use in a method of treating or suppressing cancer, wherein the method comprises administering a therapeutically effective amount of a compound of any one of claims 1-122, or of the pharmaceutical formulation of claim 123 wherein when the compound is a salt, the salt is a pharmaceutically acceptable salt, to a subject in need thereof.

125. The compound or pharmaceutical formulation for use of claim 124, wherein the cancer is selected from the group consisting of: lung, colorectal, pancreatic, bile duct, thyroid, gall bladder, uterine, mesothelioma, cervical, and bladder cancers.

126. The compound or pharmaceutical formulation for use of claim 124, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma.

127. The compound or pharmaceutical formulation for use of any one of claims 124-126, wherein the cancer is a KRAS G12C mediated cancer.

128. The compound or pharmaceutical formulation for use of any one of claims 124-127, wherein the subject has been diagnosed as having a KRAS G12C mediated cancer.

129. The compound or pharmaceutical formulation for use of any one of claims 124-128, wherein the method further comprises administering to the subject a therapeutically effective amount of an additional chemotherapeutic agent.
